SEQUENCE LISTING (2178 sequences)

SEQ ID NO: 1 - DNA, 2805 bp, Homo sapiens
atggtccgaggggggcggggctgacgtcgcgctgggaatgccctggccgagacactgaggcagggtagagagcgcttgcgggcgccgggcggagctgctgcggatcaggacccgagccgattcccgatcccgacccagatcctaacccgcgcccccgccccgccgccgccgccatgtacgacgcagagcgcggctggagcttgtccttcgcgggctgcggcttcctgggcttctaccacgtcggggcgacccgctgcctgagcgagcacgccccgcacctcctccgcgacgcgcgcatgttgttcggcgcttcggccggggcgttgcactgcgtcggcgtcctctccggtatcccgctggagcagactctgcaggtcctctcagatcttgtgcggaaggccaggagtcggaacattggcatcttccatccatccttcaacttaagcaagttcctccgacagggtctctgcaaatgcctcccggccaatgtccaccagctcatctccggcaaaataggcatctctcttaccagagtgtctgatggggaaaacgttctggtgtctgactttcggtccaaagacgaagtcgtggatgccttggtatgttcctgcttcatccccttctacagtggccttatccctccttccttcagaggcgtgcgatatgtggatggaggagtgagtgacaacgtacccttcattgatgccaaaacaaccatcaccgtgtcccccttctatggggagtacgacatctgccctaaagtcaagtccacgaactttcttcatgtggacatcaccaagctcagtctacgcctctgcacagggaacctctaccttctctcgagagcttttgtccccccggatctcaaggtgctgggagagatatgccttcgaggatatttggatgcattcaggttcttggaagagaagggcatctgcaacaggccccagccaggcctgaagtcatcctcagaagggatggatcctgaggtcgccatgcccagctgggcaaacatgagtctggattcttccccggagtcggctgccttggctgtgaggctggagggagatgagctgctagaccacctgcgtctcagcatcctgccctgggatgagagcatcctggacaccctctcgcccaggctcgctacagcactgagtgaagaaatgaaagacaaaggtggatacatgagcaagatttgcaacttgctacccattaggataatgtcttatgtaatgctgccctgtaccctgcctgtggaatctgccattgcgattgtccagagactggtgacatggcttccagatatgcccgacgatgtcctgtggttgcagtgggtgacctcacaggtgttcactcgagtgctgatgtgtctgctccccgcctccaggtcccaaatgccagtgagcagccaacaggcctccccatgcacacctgagcaggactggccctgctggactccctgctcccccaagggctgtccagcagagaccaaagcagaggccaccccgcggtccatcctcaggtccagcctgaacttcttcttgggcaataaagtacctgctggtgctgaggggctctccacctttcccagtttttcactagagaagagtctgtgagtcacttgaggaggcgagtctagcagattctttcagaggtgctaaagtttcccatctttgtgcagctacctccgcattgctgtgtagtgacccctgcctgtgacgtggaggatcccagcctctgagctgagttggttttatgaaaagctaggaagcaacctttcgcctgtgcagcggtccagcacttaactctaatacatcagcatgcgttaattcagctggttgggaaatgacaccaggaagcccagtgcagagggtcccttactgactgtttcgtggccctattaatggtcagactgttccagcatgaggttcttagaatgacaggtgtttggatgggtgggggccttgtgatggggggtaggctggcccatgtgtgatcttgtggggtggagggaagagaatagcatgatcccacttccccatgctgtgggaaggggtgcagttcgtccccaagaacgacactgcctgtcaggtggtctgcaaagatgataaccttgactactaaaaacgtctccatggcgggggtaacaagatgataatctacttaattttagaacacctttttcacctaactaaaataatgtttaaagagttttgtataaaaatgtaaggaagcgttgttacctgttgaattttgtattatgtgaatcagtgagatgttagtagaataagccttaaaaaaaaaaaaatcggttgggtgcagtggcacacggctgtaatcccagcactttgggaggccaaggttggcagatcacctgaggtcaggagttcaagaccagtctggccaacatagcaaaaccctgtctctactaaaaatacaaaaattatctgggcatggtggtgcatgcctgtaatcccagctattcggaaggctgaggcaggagaatcacttgaacccaggaggcggaggttgcggtgagctgagattgcaccatttcattccagcctgggcaacatgagtgaaagtctgactcaaaaaaaaaaaatttaaaaaacaaaataatctagtgtgcagggcattcacctcagccccccaggcaggagccaagcacagcaggagcttccgcctcctctccactggagcacacaacttgaacctggcttattttctgcagggaccagccccacatggtcagtgagtttctccccatgtgtggcgatgagagagtgtagaaataaagac

SEQ ID NO: 2 - DNA, 33500 bp, Homo sapiens
gaacactgagttcaagataagaaatgtttataactctgtgctggcagccaagagctggatcccagttgcagatggggctatgcgtagcagtgagagtgggagacacagagacagggactgagagagagagagagagagatgggatcagggataagggcagtactgccaggcactgggctgggtgctttctgttcatcacatttaacttcacaatgcccctcacacaactgttttctcccccatggtacagatataaaaagtgaagctctgagagatgaaatgatgcttgcgagtcttgtgcactggctgtctgatgccaaagcttgcagtctcaaccatgatatcatgtgcctgtcccactggaggctggatggggggctgagagagctggaccatgaaacccttgcccagctgggtcctctctttgtctctggaaagtggggagggcccccagctgctgtctacatgcagtgacgaagagggggtgagtcctcagccctgctcctgtcctgcccacatctgggtcacactggggatgacagatgtccagtgtggctggccaagaatgaatgccggtggacaggaagagaatatgaatgcctgtggctggtggcagggtgactcatctcgggccttcggtcacagcctcaaattcctgtgccagagcctggccctcctgtcattagtcctaagactggtcatgcaatgcaggagggatggatgggtacaggaggctagaggttctgaccccagctgtcccctccagaagcctcagttccgccgcagccctgagcgtgaggtgggggtggagggcagttagggcaggactgtaggtaatgcccctctccagcaaagccaacacgtcaggaatagatgggaccccctgtctgcttctcagctctcctgaccttccccgggacttaggacctgaccctgccttgctctgggcctccatctacaaagtgtctatgtgatgaggcagtcagcccaggtattcactaaggcctgaagctcccatgccctgggattcctgcccagagaaaggaggcagccctgctcccagctcccctacccctccaggcaaggcaaaagcaaggactgcagcagaatggggaagctcagttgaacatgaaccacccaaccaatccaggtgcttctcaggtgacatttggaatcctgacagcaatgtcacctgttttacaacaccaccagtcattctgcagttgaggacactgagccacaaagagggcagaggcctgaccccaaagctcacatactctgcattcctccaggtctcacggagaagggggagggcttcctggtccactccagctgggggcgctatggcccggcagaccagggtcctccagtgaggagctctggaccaaggtctaacttggctccatgtactgctgtgggcttgctccacccagtgccaggggtaggtcaaggtcaaggggcctcttaggcatctcctaagggcatgctactatccaaatgtttgtgtccccactaaaggtcatctgttgaaatcctacaccttaaggtgatgatctttggaggcgggggcctttggaaggtgatttgatgagagggtggcacccttatgaatgggattggtgcccttatcaaagaggcccagaggtgaggatgtggcaagatggcaccttctacgagccatacctctcaccagggaccaaatctgctggtatcttgaacttgggctttccagcctccagaactgtgagacagagaaataagccaggcgaagtggctcacccctgtaatcccagcattttgggaggctgaggtgggtgatgacttgaggtcaggagttctagactagcctggccaacatggtgaaaccccatctctactaaaaatacaaaaaattagtcaggcatggtggcgcatgcttgtaatcccagctactcgtgaggctgaggcaggagaatcccttgaacccgggaggaagaggctgcagtgggccaagatcgtgccattgcactccagcctggggggaaaaagatagaaataaatctcttgtttgtaagcccctccgctttaaggtgttttgttttcgcacctgaacggactaagggactaagggaccaggaatcatccaggcctcagcttcccaaagcactccccactccccagttcctacagggccccgaccccagccccagcacccgtcattcacattttttacttacaaaggaaactttgaaatggacacttcctgttactttcattcaatgaaaaaaatccccagcacttgaaggcaatcatacagaaagcctgcaacaacgcagagagtagactcttcaggtggtggtgcctgctttaggctggctctccccgggtcaaaagggacgtgataaagtgttaaagtgatgttaaggatcaagtgggcaccaactgcgactcccctcccaccccccgacttaacagatgtcaaggaaaacagaaggaagccgtttctctccaggacatgcagtgtctgctggagttttggaaaaatgctttctctcgagtcgctgcggggagctcccaggctggacccccccgcacaggtcctccgccatcgccctcccagcccccgcccccaatccccctcccagacccggtccccgcccccatcccccgccagacgtcgtccccacccccatccccctccaagccccgcccgggagggcgtcctgcccgctcattgggcctcccgccggctcatttgcatggtccgaggggggcggggctgacgtcgcgctgggaatgccctggccgagacactgaggcagggtagagagcgcttgcgggcgccgggcggagctgctgcggatcaggacccgagccgattcccgatcccgacccagatcctaacccgcgcccccgccccgccgccgccgccatgtacgacgcagagcgcggctggagcttgtccttcgcgggctgcggcttcctgggcttctaccacgtcggggcgacccgctgcctgagcgagcacgccccgcacctcctccgcgacgcgcgcatgttgttcggcgcttcggccggggcgttgcactgcgtcggcgtcctctccggtatcccgctgggtgcgtctggggacgctgcccgggctccacgtgcggagtgggtgccccctaggccggggagcgggggatccccaggggtcgcggggccctggaggagcgggcatcggacgcggacacggcggggtgcatcccgagggccccctccgaggcagatgcttcctgcgggggcgctgttcctgggcccgggaagggggcgttggaaccccgagcggtccgggccgaagcctgggactctcgtgcgtccccacccctacccccatcaggcgcccgtgcatgaagggagaccctcacctccggactgagagtcggagcgtctcggagcgacggggagtagggagcgggacccggggcggagggtagtgctggcccctgcggactccgggtcccctgtgtcctctcgggaggggctggacgggctgagctgccgaggggccgatttgccctgggccggacaaagagtggggctttggccggtcccccacggtgggctccttccctctggggattgagggactcaagacaccccgcgcctgcgcttttcttttctttttttctttttttttttttgagacggagtttcgctcagtcgcccaggctggagtgcagtggcgtgatctcaactcactgcaagctccacctcccaggttcacgccattctcctgcctcagcctcccgagtagctgggactacaggcgccagccaccaagcccggctaattttttgtattttttagtagagacggggtttcaccgtgttagccaggatggtctcgatctcctgacctcgtgatctgcccacctcggcctcccagaatgctggggttacaggcgtgagccactgctccctgctgcctacgctctctgggtcgcagcccagccttctgggggctgggtagcctcccagaagggcaaccctgggcatcctccagggcaggctaactggagtctagtggggaggggtaccttgaaagaggaaagttgtttcctcctcctcctcctcctccagtgtttgggacccttcctgggggctggagtgcatccctggacaccccccaatcccatcctcttctctagtttccactgacctaggcccaccctcccctctccggctcagtactcctggaaatgagattccgtacatttgaatcttgtcctaatgaaatatttgtccatgtgggtacctgtgtgtgtgtggtgggggtgcagacggagggtttgtttctcactagctggaactactggggtgtggtatgcttcctgggaatttgtgtgccacagtcctggaggcgaggagggggttgtgagccagtaggcaggggctggggcaagtagcattgtgaagctattgacacccagacgtccccaggcaggagattatgcccccattagcccccttttatctgggcttccttaacaatggactctttgccctgcctgccagagccagcagggagtgactgttcagtggtgaggaagcgggcagaggaagccctgccattgggtaggagcagtgggcagcccctgggctgactgggaggtggggattagggattagacagtcctggctgtctgccttcccctaagccagggggagaggagcaaagggcacgaaatgtggcctccaggaggattagaccgccacatgatcatttgcacaccctggggtttagcaacaataaaagtcagcttttttgtatcccaaggtggcctgtggacacccacatggacaaatgtttacactgggacagaattcaaatgcagaggtcccaggagcctaaagtacactcactctggtatagaaaggattccttactgggcagaggacaggtgcagcctggggctttcccaggcaggacacagggaggctcaggaaccaccaagtccctggaaggtggatctggaggcgttggcaggagccactccctgggttccagggctccaggttcctgctttaaccccctgtctcacagagggctgtgcacttgggggctgctgagcatgtcccagaggctgcatcctggacacagcacctcagtgcatctgagctgaggctaacttggcaggagggacaggcagaacctgccagccacgtgcaattccacccctctggccactcagggaaggagagctgtgagtcaagatcagatttgggtcaggacaggctggggcctgcctgtccctgtgcatcccaagatttatggctggccaggggttgggctgggaggggtggtcttgcatgccaggagagtgcagatcagcctgagaggccaggccagtaagtgaggtcagatctcctgcacctgatagcattaaggccatctacaccaaagctctaatgctgatatgttcctggcctctatgtggggcatggaggtggggcatggaggtgaggcctgctcgcctgggcttctggaagtgggagactcattcctgtggctgaggcctacagcagtgctgtgtggtaggaatacactggaagccatgatgtcattgtgcattttctagaagccacattgaataaagtaaaagacacaggtagaattaatttcattgagcccaatatatccaaaataatatcattttcacatctattcaatataaaaatttactaatgagatatttcatactaagccactgaaatccagtttgtatcttacacatctcagttttgacgagccacatttcaagggcgtgatagccacatgtggctcccatagtagacagtactggtctagagaaatgttggtggcatccttgctgtctggtttctggccttgccaaaagtattaccatcccagtgtggtacattctttcatgtatttgtctcctgtccccagagcagactctgcaggtcctctcagatcttgtgcggaaggccaggagtcggaacattggcatcttccatccatccttcaacttaagcaagttcctccgacagggtctctgcaaatgcctcccggccaatgtccaccagctcatctccggcaaaataggcatctctcttaccagagtgtctgatggggaaaacgttctggtgtctgactttcggtccaaagacgaagtcgtggatgtaagcagtttgcttatctggacgttgtcaagttagaaaagctgttttgggatgggtgtggtggctcatgcctgtcatcccggcactttgggaggccgaagcgggtgggttgcttgagcccaggagctcgagaccaacatgatgaaacccagtctctacaaaaattacagaaaaattagctaggcatggtgttgtgggcccatagtcccagctactagggaggctgaggcaggagaattgcttgagcctgggaggtggaggttgcagtaagtcatgatcatgccactgtactccagcccgggtgacagtgagatgctgtctggaaaaaaaaaaaaaagaaagactgttttgttttggaagcaacacaggcagttgtaggccccctgtgccagagtgacataaactctgtacacctccagtgatttggtccatgtttgtaaaccctgaatgttccagggcagtttcttttcttcactttttatctcttttttttgggtgggggggcggggtacagagtcttgctctgtctcccaggctggagtgcagtggcgcaatctcaacctcccgaggagctgggactacaggcacaggccatcacaccttgctaatgtttgtactttttgtagagacggggttttgccctgttgcccaggctggtcccaaactcctgcacccaagtaatctgcccacctctgcctggcagttacaatttcaaataattcctccctttccttcaacacttggctcatgaccgtccagtccaaggaacctgtcctgcaggtgtgcctctcccgagcttcctctatgcatcttccataatgaagatgccttctcactggaaaccctacaagggtgggaacgtgccttatttgcctgtatcctcagggtctagcagagagaagataatttgtaataccaaaacaccattaaattcagctgatgctttcataagcgctccttggaggaaggactccatttacttgacagatctgtgcaagacagcagcctggcgcgtctaacctgcagccagttgcatcctctgtttaaccttgtttgcggaagctttctctaaacagccagcacttgtctgttcccacatgggtccgttctcccagtgaatcaccgtggtgcctgctgactgctctgtagcacagtgcttcgcaaagtgtgatcctgggaccagcagagcagcagctcctttgagcttattggaatggcagaccctcaggtcccacctctgacctgctgcatgggaattctggggagggacgcagaatctctggttccacaggctctccggtgatgctaatgaataccggcatttgaacagcaccgatctagcccctttcagtccatgagccaacaacccttggtcctgtctgtggtgacccagtgtgactctcatggggagcaaggagaggaagttgaagttcactgacagggttgttaaggggattatgcaatagatgagacccatgggcctgaagtccgagggtgtatgttagttccccgttcttttgacccatggattaacctactctgtgcaaagggcattttcaagtttgttgccctgctcacttggagaaagcttatgaaggatcaggaaaattaaaagggtgctctcgcctataacttctctctcctttgctttcacaggccttggtatgttcctgcttcatccccttctacagtggccttatccctccttccttcagaggcgtggtaagtcggctttctctgctagcgctgagtcctgggggcctctgaagtgtgctcacacatctcctgcctgcagggcactggtgtcgggcacctcagggtctgtcccatggtggagccccatgcctcactgcctttcagacagagtagccacagctggccctatttccaggctacccgggcagcaaaacttactgcatgtgtaattaattatttggctatctgtaaggtaaactggctggttcacttaatctgcaccttaagcatcagatagcttctcagtgatctagttaaactatatgatgttggccaggcgcggtggctcatgtctgtaatcccagcactttgggagcctgaagcaggcagatcacttgaggtcaggagttcgagaccagcctggccaacagtgtgaaactctgtctctcctaaaaatacaaaaattagctgggcatggtggtgtgcacctgtaatcccagctgctcgggaggctgaggcaggagaattgcttgaacttgggaggcggaagttgcagtgagccaagatcgcaccactgcactccatcctgggtgacagagcgagactctatctcaaaaagaaaaaaaaaaaaaaggtaaataaagtatatgacactgaagaatctgttacccctggaaggtggagctttactcttagggggaactataacagtcatatatatatatttttttcttttctttttttttttttttgagatggagtctggctctgtcgcccaggctggagtgcagtggtgcaatctcggctcactgcaacctccacctcacaggttcaggcaattctcctgcctcaacctcccgagtagctgggattacaggtgcctgccgtcacgccaagctaatttttgtatttttagtagagacagggtttcatcatattggccaggctggtctccaactcctgacctcaggtgatccgcccgccttggcctcccaaagtgctgagattacaggcgtgagccatggtgcccggccaacaatcacatgtgttgtaaacaacaacaaaaatctgtcagcctggtctaacctagatttgtgctttgttttgttttgccactttgtgatgcacaggaggaagtttaggctgtaaaatactagccttttagggtaatttttgaactcacaagagcagcagcggaacctttgatgcaatcctgtatgtagcaccagcagagccacgtggcagagggactcgcattaggagcctcccattacagactacgtgctcctgtgcgttatcttatagggtccccacaaccaaggggagatgtgattattcatcctgtgtggctgtggggaacttgagagtcatacttgcccaaagagcacggccagcgagcttgcacccaggtcactctctgctcctctgtcagaacagggcatgtcttggttcactgcagggcggctcttctcattctctgtagtttggggtccaggatagtggtccacggagccactggagtgcccagctactgagtgaccaaagcatattttggatttccgacattgccacagcatggttgggcatcagcaggaccccaaccccttgttatgctggtggctttatgtggttatttgatcttccccagaactcagcaggagtgcacccagcagcaccgtagtgatgctctctggctccccagtgcacggttctggctttccttcctggtcgagagtttcaagccctctgggtcctactctgtccttttcagcccatagctttgttcaaaagctgctggcagtgttcagatttggctgagttcagtgaatatgtgcattggctgatttctgagccatgccagggggatggagaagccgaagcaggagtgtttgttctgcaggctctggagtaggcattgggtctgtgccggctcacttgctagtcttgcatccttccccaaccccctctggggatgtctggccacatcagaagacagtttgggttgtcagaactgggggagtaccaggccgaggtgggtggatcatgaggtcaggagatcgagaccatcctggctaacacagtgaaacctcatctctactaaacatacgaaaaaaattagctgggcgtggtggcgggcgcctgtagtcccagctactcgggaggctgaggcaggagaatggtgtgaacccggggggcggagcttgcagtgagctgagatcctgccactgcactccagcctgggcaacaaagcgagactccgtctcacaaacaaaacaaaacaaaacaaaacaaaatctgggggagtgccactggcatctgatgtatagaggcccgagatgctgtgtcatcacccgttgagtgcgctcataggcatcttcctgacaattagaacccattattcttcaaattcaatgcaagcaaattcaaagcattactgtgtacataccgcatgctaatcaattgcaccactggagctcctaaattcaaaacattactataaaaaagttcaaaatgcatggaaaagttgtacatggcaggagaatatttgggcttctgactaccccttgaatgaagatgatccaccagccgccttcctccttggtcttcactccagattcctagcatttcattctgtgtctctttatgcagtgaggtttttgtttgttttttgagacagagtctcactgtatcacctaggcctggagtgcagtggcgcgatctcagctcactgcaaccctcggctcctgggtttaagcgattctcctgcctcagcctcccgagcagctgagattacaagcacacatccccatgcccagctaatttttgtatttttagcagagacagggtttcaccatgttgcccaggctggtctcgaactcctggcctcaagtgatccatgtgcctcagccttccaaagtgctgggattacaggcgtgagccaccatgcccagctcctagtgaggtttttgatgccttgctacatctgccctagaaattgtgtgactacgattttggaaatgttgctgtgtaaacttgtgatcatttctggactccaggcaagaatcttgatggctaaggtgtggctgaacatgtctgattctctcctggacctgttttaggccaaactctgctctgaaattcctccgtgtggaagggcgggctggggagagcctcccagctggaatcttttggatgcctttctctgtgggtatctgatggctggctctgatggctggctgtgatggctgtggctggaaatcattgttgacatgagtttcacagatgcaggctctgtccaaattgtagcaaaagctgcctgccccagccgagctatgggcaataaggtggtttaaggatatagatgaaggaaaactcacccttagaataatttatccaaaatgctgctgtgttgtgggttagaggacattttctgaggtcccaggttcattgtttcatttaagtctcaaaagtccctccaggtgttggttctaattgtcaaagcatggggggagatgggctcatgggttaaaggtcttatcccagatttctgtatcctccttgcaagcagcaaaggggtctggatttgaatccatgaccatgtttctcctttgggtttccatcacactctgtccccgtgcactgagcaccctttagttcatatgacccccttaggcatgttacatgggcactcctataggtgcccatctggccctaggacttggccaacacaacatggactccagtttccatctgcctctttgccaggcacttttgtgcagtgcacacactgtacaacagtagacggcaaccctgagagccagagtagagcctgtcctagcaccggaatgctcggtaaggatttgtcgcaggagtgattccaaagccaatgtcctccctccatatcagcctgtttgtggctctgagaagctctgcccacatgtgaaagcttgttaagcacttaagcactaacccagagcttcagacagtgccagtcctttttccccttctttaaaagcgatatgtggatggaggagtgagtgacaacgtacccttcattgatgccaaaacaaccatcaccgtgtcccccttctatggggagtacgacatctgccctaaagtcaagtccacgaactttcttcatgtggacatcaccaagctcagtctacgcctctgcacagggaacctctaccttctctcgagagcttttgtccccccggatctcaaggtgagttggtggtgagggggcaggtgttctggggtgcagctcttctttgcctccctgattgccaggagctaccagttactgtctgcacaatcaaacagaaatagacctgtccttgatggttaacggaaataaaaggcgcttgtcccagaagctcaggtgaggcaccaccctgattatgggaatcacctgggaacatatacccagacctaaaactcagatccacttcccaggctgtggttatatagtcaggggggtgcagtatgggtattaggattttttattttttagttataaagatttttttttggtttgtttttgagacagggtcttgctctgccgcttaggctggagtgcagtggtgcaatcatagctcactgaagcctcagactcctgggttcaagcagtcctcccacctcagcctcctaaggagctgggacccacaggcatgcagcaccacacctggctaatttttaaaaattttgtggagtgttgcccaggctggtctcacactcctggcctcaagcgatcctcccaccccagcctcccaatgtgttgggattacaggcatgagccattgtacccagccactaagatgattcttatttggaaacacggtcaagaacaactgcgttcggtagtttaaccttttttgattgtggtggttttagtatgccttaccactctaccatagtaagaaatttgcagaccatgtacaccaacctttggtgctcctggggagaaagaaagaaggctatgcaatgcaatgcatgctcacagtccaagggagagggaaagctgtctaacaggattggttttcccgtgtgctttataagcagatgagtagaggagacagctcttattgtcctagtggcaattgggataggctgcaaagtttgttagggtggaggcttattccgggaccaagggagcccaaagaaacaagctcctgccaggcgcggtggctcacgcctgtaatcccagcactttgggaggctgaggcaggtggatcacctgaggtcaggagtttgagaccagcctggccaacatggtgaaaccccgtctccatgaaaaatacaaaaattacccgggcatggtggcgggcacctgtaatcccagctactagggaggctgaggcaggaaaatggcttgaacctcggaagtggaggtggccgttagccgagatcacgccactgcactccagcctgggcaacagagcaagactctgccttaaaaaaaaaaaaaaaaaaaagaaaagtaaaaggaaaaaaaagaggctctggcctgctggggtgcctgcaaagtctccgtggaagggtgacattcaagccgagacctccagggaactgtctcctgggagcacagagccctttgctcagcccccaggtggctcagtgcccccagccagcagactcagagcttgcatgattctttggtgctctctgcggtcttccaatgatgctgaaataaatggtgcttggtgtctccctgctgtagtccccttgcttgctttgctcacaggtgctgggagagatatgccttcgaggatatttggatgcattcaggttcttggaagagaagggtatgtatgggctgggaggatcagccatgcccttttgacaagcatttactagcggtcttggtaaagacttgagatttgccttagttctaacacttagtgcccaacgccttccttgtgttgctcaacctactcatgagcccaggagataggaaatctccgtcccattgtacagatggggaaacagaattttggaaaggagagccaagcagcacacacccctccctgaggggcagagccgagatttgaactgggatgtcatgactccagggccctctccctccccagggtccccttatctgaaggcggtttttctttccagctcgacctcttgtgacccttagtttaacaagggccgaagttaaagagtttctgcgcctggaccccaaatgaagcaatcagatttctcatctccagtcaggtgtgggtccaagcccactagacaagtttgctcttcccagagcacatttctgccttcaagtcatcctggcttgtcagggctgggggagttctgctgtagaaatattagagtggaaggaaaaagatgtgttgggagctatttttctttaatactaaaagttggttgatgaatttgtcgttggccaagaccaaggagactgcatttttaaggacatatgtgtatttatctgctcagaaaatgttcattgctgtgtgctagggatactgcagtgaacacagaggtgtgacccttgccagccttgtgagagaagtgagcagataagtaagcagaagggtgatgctgtgtcgatgggaaagtacaggtgccaatgagaaggcacaggtgtcaaggagaagacacaggatgctggaggctcatgcaggatggatctccaaggcccaggggaagaagggcctctcggaggacgtgaatccacattaagactttggggataagtaggagcgccttaggcatggggacccatggatgcgaggcctgtaggacacagacaggatggcatgaaggcctgtgcaactggaggggtggggatggggacactaagagatggctggaagtgtgggggtggggacactaagagatgactggagaagagggggtcaggagtggtgaaaaatgggagaggagggcaggctgggccttttggatacagggggattgcatcctgcagtggtagggagccactgagggctgctgcagtaggagtgaggggatcagaggagagctttggaagccccctggatgcgggacaggaagcgagataccagtgtctaggaggccagtgaggcagccacaggctccaccaggatcagggctgcgagggtcatgaggaggaaaccaatttgaaggagtccaggggaataggacttggaaatgaccgatgggacatttgggaagaggaagacagaagagcgcagtcccggcttctggctttagcagttgggcaaggggagatggggagatgtgcccatgggttgagggttgaggacattaggagggagccggtatggcaggaagagctggtgtgccagagatgctggaagcagcatctgcctgagaacagatacctggcaatattcctaagggaaagtgacatctcggagggtgaggagggcatctgatagggcctggaaagagccggggcaagcatgaatgtgaggttatcttggggggcaaggctcaggcgttgaggagcagcccctggtctcttcagcctgaagttggaagccagagttgggccaggtgcagctgtggttgtctgaagtccccctcccccagcccagtgtgccaatgctgtaagagcaagggccgctcactggtgctggtggctgagtcccagcacccaggacagggcctggcacatactggtgcccaatcctcccttctgggtgcttcttccaaggccttgtgatggaagtgagtaccctcttcgacatcagacccagcttcaaatcctggctctgctatgtattggctgcgtggctttagacaagtcttttaaccttgctgtgcttctgatttctcagctgaaaaatggagatgatgatagtggtttctgtaaggccttatggtgaagcacctagctcagggcctggaaggcaggtgtaaccagtggttcagttgttataaaccaacactaaccctcgcctttgcacctcatgaaaccagatatgtagatggagcccacaaagctagcaggagccaagctcacgtgtgtcctgctttaaagccccatacccctttctccgggtgacaaacacctgtgctcgttctcttcccttcccctcttccccttgcatttggctaataacaggccagctgcctgcctccctgcagtttggtagatgggtgggtaacgaccaccactcccacgttcgcctgatgggcttgttttccgtgcccttcacaggcatctgcaacaggccccagccaggcctgaagtcatcctcagaagggatggatcctgaggtcgccatgcccagctgggcaaacatgagtctggattcttccccggagtcggctgccttggctgtgaggctggagggagatgagctgctagaccacctgcgtctcagcatcctgccctgggatgagagcatcctggacaccctctcgcccaggctcgctacaggtacccactcctcggggtgagcacgggcagcaccttgttttctttcttgtgcattatggaggaagatggtactgccacatgggagcgatagggtgaggcaaccatgacaggtggttgggaacatctccttccatgtgtacagcctgggctgctgccatcactcccagcacagcccccaacccccccaatcctggaaccttgccaagtctcccttcccatggggtcatgaccaggaggaaaacaaactccagctgagccccttggggttccccatataggctcctgcctgtggcagctgggccctctgtacccctttccaactctgtctccctaacatggcacctgagctcctgccatcctggatttcatggaccccaaggatgggggtcctgcatctgggacttggcctattactcggagctccttttcagccgcctccctccacctgtccacccacctcaaggctcctttcttgagacctctcctaatttctcccttcccctaaacccacaattttgaacctccatcgaatggtgctgtattttataatgtcatcaaatatcaaatggagacagtgctatggtccaaatgattgtgtaccccccagaatttgtcttttgaaatcctaacccccaacatgatggtcttaggaggtggggcctttgggaggagattaggtcatgaggaaagggctgtcatgaatgggattggtgcccttattaaacagacccaagagaggtcccttgtcccttctactgtgtgaggactcagaaggtggtgtctatgaagaagcaggccctcaccagacaccaacatgtctgctgccccttgatctgggaccttgcagcctctagaactctgaaaaatcgatgtttgttgttttataagccactcagttggtggcattttgttagagtagcctgaacacggactaagtcaaacagaagaacccacaaaccagctacagagttgggcatttggagaaattcaaaaatgagtcagacataactccttattcttgaggtgccctaagagatgggacacagcagctgcccaggtgcattagtttgttctcacattgctataaagaaatacctgagactgggtaactcataaagaaagaggttgaattggctcacagttgcacaggctggacaggaagcatggtgctggcatctgctcagcttctggggaggcctcaggaaacttacaatcatggcagaaggtgaacgggaagcatgcacatcccatgactggagcaggagtgagagagagagggaaatagagggaaggtgccatacacttttaaacaaccagatctcatgagaacacattcactatcaagagaacagcaccagtggggaaatccgcccccatgatccaatcacctcccatcaggctccgcctccaacactgggaattacaatttgacatgagatgtgggcagggacacagatccaaaccatatgaccagattaatacgatttgaggcatcacgaggtcattaaagagagggaataaaagactggggctccaggaagaaggctctggaatccagcagagggtcaaggaccagcttgtaaagctggtggtgcctgagaagtacctaggagaacatagatgctgtgacgtttgatgtagctgttttttgttttgtgttttggtttttgagacagagtctcactctgttgcccaggctggagtgtgcagtggcgtgatcttggctcactggagcctccatctcccaggttcaaatgatcctcatgcctcagcctcctgagttgctgggattacaggtgcacaccaccacgcctggctaatttttgtgttttcagtagagacagggtttcaccatgttggccaggctggtcttgaactcctgacctcaagtgatccaacaacttcagcctcccaaagtgctgggatgacaggcatgagccaccatgcccagcctgatgtagctgtttctgtgcacattatttgctgtggggtatattcagatttcttaatacaagatgattctttgcctcatgacttacacaccattttctatttaatttcagctatgatattggaaatggacatgtcttttcaaggaaaataaaagcaggctttctggaatggcgacttccaaacatatttgtcaatttaaaggagctgggagtggggaccctatgctccgtaagcactctcttagctgttcttggctgtgctccccgcttcagcttcacactgcccttgctgtgaagggagcagcctgggccgggcgcggtggcttacacctgtaatcctagcactttgggaggccgaggtgggtggatcacctgaggtcaggagttcaagaccagcctggccaacatggtgaaactccatctctactaaaaatacaaaaaattagctgggcatggtggcaggtgcctgtaatcccagctacttgggaggctgaggcagaagaatcgcttgaacccaggaggcggaggttgcagtgagccgagattgcgccattgcactccagcctgggggcaacaagagcaaaactctgtctggaaaaaaaagaaaggagcagcttggcaaaccccaccttgtcgcttttgtgagtgcctctgaccctttggctgccaggacgggcgtattttatggaaatgctaagcaccaacagagtaaagtggtttggtttttcacagtggtgggagataatagctccaaattgtctttttcagcactgagtgaagaaatgaaagacaaaggtggatacatgagcaagatttgcaacttgctacccattaggataatgtcttatgtaatgctgccctgtaccctgcctgtggaatctgccattgcgattgtccagaggtgagcattttaggtggctccgtgtcttcctcacagggttgatatgaggatgaaacaagatgatagatcatggtggcatgtagtctgggacctggattgtcgtgccacagatcacagctcacagtctatgtgcaatgcccctgaatgttgcccacctgtcctcaagccacacatgcacctgtaactcagtgcaagcccagaaactccccgtggggactcctagagctgtcagtggcctcacatagcagctggtccagtctcttgtgattgcccaaggaaactgaggcctggagagcttggggtcactgctctgaggccatagagatgcctagtagaagggccaggcctagaagcaggatccttgctgcccctctgagctgtttccatttaaaatcacatgaaggccggcgccgtggctcacggctgtaatcccagcattttgggaggccaaggtgggtggatcatgtgaggtcaggagtttgagaccagcttggccaacatggtgaaatgccatctgtactaaaaatacaaaaattagtggagcatggtggcacgtgcctgtactcccagctacttggaaggctggggcagaagaatcgcttgagcctgggaggcagaggttgtagtgagccaagattgtaccactgcactccagcctgggtgacaggagagaaaccctatctcaaaataaaatgaaaggtaatgaaatgaataaaataataaatcaagtcacggccgggcacggtggctcacacctgtaatcccagcgctttgggaggccgaggtgggtggataatgaggtcaggagttcaagaccagcctggccaacatggtgaaaccatgtctctactaaaaatacaaaaattagctgggcatggtggtgcatgcctgtaatcccagctactccggaggctaaggcaggagaattgcttgaagcaggacctaggaggcggaggttggttgcagtgagccgagatcatgccactgcactctagcctgggctacagagcgaaactccgactcaaaaaaaaaaaaaaaaaaaaatcaaatcacatgaaagtagaacatagggaattccatctttcgttctaggcatagtttgttaatatgattcagagccagcagttaggagaacacagtgtgactctcctagaacttcttgattgggcttcctctgattgggtttcctctgattgggcttcctctgaaagtgggggggatggggggtggggagcagaatggtcagagcttggctcagcagtcagactgctcttcttcaaatcctggctgcattgcttactacagctgtgtgactccagatgactgaatccacctctctgtgctgcagcttcccgtctagagagatcacctggagcagagggtggtcaggagactcaatctggttactgactcacagtgcaggagtactcatcccatagtaagcatccagctagagatgttgatttctattttcaggtaataatgatgatcgtaaaattagagacagataaaaggtatgggcattaggccagggcactgcaatttctaagctgtgtgacctcaggcaagttactcgacttctctgagcctcagcggtttcatccgcaatatatggataggaaaaccgacctcagtgggttgtctgacagtggagggcacttgattaaaaaaaaaaaaattaccctggtctgaatattaccctggactgaaagaaaaatattgagctaatacaggcatcaggaatggggctgcagggagtccagggaagggagaacgaagagcctgaaggtgtgaggaggtgcgagtgctgatctgtctgctacaaagaggctgctgagcctcctgtggatgtggccctggacttggcagtttaatacctgagctgttaaaataacctcagatgctgtgttctttaaggggtaggattcagattcctgctgaaatgcttctgaaagggagggaatgagccagcccatccccagttgctttttaagatcattgggaagttctggtcttgccatttgtccctggaccactcttaggtcctcctgccccacttccatctgggtgtgtgccctgggctgtccaccacacagctacatcctgccatcttccctcctggagccactgtgccatgcatggatctgtagcttcatttttcttggcttttccctggtttttctggagcagagtctctagtaaactcccaaggaagaaaacgtttgactttatgtgtgttgggaaacgtgctttttttctattacatctcagtgataggttggccatgtctagaattgcaggttgaaaatcatttcctctcagtatattggttagtgagaagcctgggactgagacagtcacattctcacttctttgcaggtgagtgctcttaggactgtctttttatcccttatactctgaaatgtcatatgtcttggtgtaagtccttatttcagttattgagctggacaagtactggagaccccttcagtcaaagccttctgtcattctccagctctaggaaattatcttctattgttatttctgttattccttcccttccattttcttttttctttttttttttttttttttgagacagggtcttactctggtgcccaggctggaatgcagtgacctgatcatggtacactgcagcctgaacctcccagactcaagtgatcctcccacctcaacctcctaagtagctgggactgcaagcacacatcaccacacccaacaaatattttttaaaaattttgtaagatgggatcttactatgttgcccagactttttcttcctcttcctggggctcttattaggaagatgtttgacttcctgggttggattcctgtctccgtgtctgactttctctctttgtcatatttttcatcactcgttgtctttttgcgtctgctctgacagatttcctcaaattttgtcttctagtcctatcctacagtttttactttcagcaaatataatttaatctccaagagtactctcttgttcttttttcttagcattctgttcttgttttatggatgtaacattctcttggaatatttgctgtcctctagatcatcccttctccatttcttcttgggctagtttttctgtttcttcatctttctcttttatgctacttattctgggcgtgttcttggtgggttttttcccatatagcaacagaggacttggagctcagggagaaaagggtaggtgcatcacctggcagagctcccagacagtgacaggcaggctgcgggaaggatgtctacttggcggtgctaccgctttcctagaaaccctttccctggagctggttgaactgttgggttttgccctggtggtgaacgctggctccccgtgctctgcctgtttcatcaccagccccctccccttctgcctggggtccagtaatctgttgaaatatatatcttgctcattggtgagctcctgctccttcctcgttgctcttgcagatttatcacttctcgtaaggctgcgcttgtacttcgggattttctctgtgccacactgggaaacatagggtggttgcatgctgcagtcctgagcacttatttcactcacatctttacacgaagatttggtgggtgtttactttgtttttagtaagttagtctgtcatgtcctttgatcctttttttttgttttttgagatggagtctctctgtgtcctccaggctggagtgcaatgtcgcgatctcagctcactgcaacctccacctcctgggctcaagagattctcctgcttcagtctcctgagtagctgggattacaggcatgtgccaccacacctggctaatttttgtatttttagtagaggtggggtttggcatgttggccagcctggtctcaaactcctgacctcctgacctgcctgccttggcctcccaaagtgctgggattacaggtgtgagccaccacacctggccctgattaatcttttaatgcccagtctctccttcaaaagccggctcctttctctccctcgccttcctagattccttctccactccccaggatcagcctcctcctccccaccccaccactgccggggggatgtctgtggtcaggcatttatcagagaccctgaggtgggggtcctttatgtgtctgggggatggagagtctagaggaggtagcgttcagacctctccatggtgcctctgctgggctcacatgtgaccaagcacagcaaaccatgaggcaggggatggtcttgaccatgagagcccttgcagcagctgccatgggcctcagctcctctccaagctgggaagagccctgaaaagccaaggtgtttttttttccctctttatttcagtgtaagtcccttgagctttcttgaaccagaagtgggctcattttgctttagagatttcaggtgggcttgtccttgtcctagcatcccagatccaccttctgggaagtcatcagattggaggtgatgttggcagcttttgtaaacaaagggtagtgttgtaagctgttgtgtctgcctatgtgtgtgtttgtgtacttggtctcatctctgcagactggtgacatggcttccagatatgcccgacgatgtcctgtggttgcagtgggtgacctcacaggtgttcactcgagtgctgatgtgtctgctccccgcctccaggtaaatactttggctgtgggtgtgtgggccggacgggcacctctctcatctgatgaggcctcacacgacattctagaaacagctggctgaacaccaagcaaggagcttgcccttgggtgtggggaccctgtctcatgggaggcagctgagtcagtcagaggtcctggcacacctgctgagagctgccacccaggccaacctgaaccggagcctgggaagacttcccgtcggatgagtctctttgagtgcagcattgatggtggaagagcagagaggccccagataagcagggaaaggtgcttcagacagagtggctgggatgaggactggggagtgtcagatagcgctggcgtgtctgagcgaaggagctctggcacccatggcacaggaaggaggtgggaccctggaggggcagggctagcagagctcctcggagcgtgtggctaggtgcctggtaatgcaagccccctgtcctccaccctctgttgtactgagtcacagtctccggggtgaagcctagcagtctgcgttgacaggccccaggggatgccgctacttcctgaattctgaattctggaaactgagccggagttcagggcctggctcccattaccagggttgggcgttatcctgaaaatcataggccttggtttcctcacttggctaacaggggtgatccccatcccctcaatgggtttccgtgagctcctgagagcccgtagcatggtacttggcacatgctgggcatcaggaggtatggcctctcttgctattgttgttattggtagacacagaaggatttaaaagtaggggaatgcaaagatccgatttgctagggaagagggcagtagtggccaagtagagggtggatcctgggccctggctggcagcaggcagcaaggggggctgccagggcccaggcagggacgatctgtagaccgagaggcttcctaaggctcttggacaggaggaggtgtcggttccaagcctaaggagtggggcagccctggtgactggtggtcagtggtgccaggcggtgggtggtaggacaccctggcaggcaagtaggtttgtgtgggggaaactgataggcccctccagggattcgttggtggacaacacctgtgatgtccagtgggaggtgtccaggtagctgggagggccacaggcttggaagacctaggtggtgacatcagcccagcactgagggctagaagaagctgtgtctctggctgtgacggcaccctagagtgtgtgtggtgccctctactggccggcaatgtgggtccaccgtagctcagactgcacactgcagcagcgggaacggcctctaagccaacttcctccatgtgtttcaggtcccaaatgccagtgagcagccaacaggcctccccatgcacacctgagcaggactggccctgctggactccctgctcccccaagggctgtccagcagagaccaaagcagaggccaccccgcggtccatcctcaggtccagcctgaacttcttcttgggcaataaagtacctgctggtgctgaggggctctccacctttcccagtttttcactagagaagagtctgtgagtcacttgaggaggcgagtctagcagattctttcagaggtgctaaagtttcccatctttgtgcagctacctccgcattgctgtgtagtgacccctgcctgtgacgtggaggatcccagcctctgagctgagttggttttatgaaaagctaggaagcaacctttcgcctgtgcagcggtccagcacttaactctaatacatcagcatgcgttaattcagctggttgggaaatgacaccaggaagcccagtgcagagggtcccttactgactgtttcgtggccctattaatggtcagactgttccagcatgaggttcttagaatgacaggtgtttggatgggtgggggccttgtgatggggggtaggctggcccatgtgtgatcttgtggggtggagggaagagaatagcatgatcccacttccccatgctgtgggaaggggtgcagttcgtccccaagaacgacactgcctgtcaggtggtctgcaaagatgataaccttgactactaaaaacgtctccatggcgggggtaacaagatgataatctacttaattttagaacacctttttcacctaactaaaataatgtttaaagagttttgtataaaaatgtaaggaagcgttgttacctgttgaattttgtattatgtgaatcagtgagatgttagtagaataagccttaaaaaaaaaaaaatcggttgggtgcagtggcacacggctgtaatcccagcactttgggaggccaaggttggcagatcacctgaggtcaggagttcaagaccagtctggccaacatagcaaaaccctgtctctactaaaaatacaaaaattatctgggcatggtggtgcatgcctgtaatcccagctattcggaaggctgaggcaggagaatcacttgaacccaggaggcggaggttgcggtgagctgagattgcaccatttcattccagcctgggcaacatgagtgaaagtctgactcaaaaaaaaaaaatttaaaaaacaaaataatctagtgtgcagggcattcacctcagccccccaggcaggagccaagcacagcaggagcttccgcctcctctccactggagcacacaacttgaacctggcttattttctgcagggaccagccccacatggtcagtgagtttctccccatgtgtggcgatgagagagtgtagaaataaagacacaagacaaagagataaaagaaaagacagctgggcccgggggaccactaccaccaatgcgcggagaccggtagtggccctgaatgtctggctgcgctgttatttattggatacaaagcaaaaggggcagggtaaagagtgtgactcatctccaatgataggtaaggtcacgtgggtcacgtgtccactggacaggggtcccttccctgcctggcagctgaggcagagagagagaggagacaaagagaaagacagcttacgccattatttctgcatatcagagacttttagtactttcaataatttactactgctatctagaaggcagagccaggtgtacaggatggaacatgaaggtggactaggagcgtgaccactgaagcacagcatcacagggagacagttaggcctccagataactgtgggcgagcctgactgatgccaggccctccacaagaggtggaagagcagagtcttctctaaactcccccagggaaagggagactctctttcctcgtctgctaagtagcgggtgttgttccttgacactttttgctaccgctagaccacggtccgcctggcaatgggcgtcttcccagatgttggtgtcaccgctagaccaaggagccctctgatggccctgtccaggcataacagaaggctcgcactcctgtcttctggtcacttctcgctacgtcccctcagctcctatctctgtatggcctggtttttcctaggttatgattgtagagtgaggattattataatattggaataaagagtaattgctacaaataatgattaatgatattcatatataatcatatctaagatctatatctggtataactattcttgttttatattttattatactggaacagctcgtgtcctcagtctcttgcctcagcacctgggtggtttgccacccacaattttccacatgcttctggtctgcgttctttttttctccttgcatcttctcattctctgatcaccccaacctctctctgtcttccctactctgccagccttgatggagacaagcccttgagaccagaactcaccttttccccaggtgtgataatctattagcaaggtcagtgtaatatgactgacggtgaaacgtgtatttttttctatttatgcatttgagtacagtacgtacaagaaaaataatggtgtgctcaaactgttaaatgttggaaaagaaagatacaacccttacccatattgtgtaagtgccctggagtagaagaaccagcaagctcagacaaagcacttgactgagaagacagaccctttaaaggaaacgggttctagggacaaactctacgtgggcctgttctcttgataagaccgtgaactctttgagaaaagaggctacttgtgaaaataatgagcccccttcggggcaggagttccggggtttgaacctgccttcttacatcttgagggctaagtgagttcccaaggcctctgttcagtggttgctccgtcagtgagctcaggtctggtgagtggcagggtcttccacccccaaccccaccgggtgtcagagcaagacactgtcccccatggagctggaatggggtggaggagcccacatctggcacccacgtggcctccttgtgacggacccacccttgcaatgttggaaaggaaagttacaagtttcttttcccaagtttcccagtaggctttgttctgttagcttcacgccttcggtcattagcagacataaataaactttaaccattgttttttatcttttttttttttttttgagatggagtttcactcttgttgcccaggctggagtacaatggcacaatctcggctcaccacaacctccgcctcccgggttcaaacgtttctcctgcctcagcctcccaagtagatgccaagcctcccaagtagatacaggcatgtgccaccacgcccagctaattttttgtatttttagtagagatggggtttctccaggttggtcaggctgatctcgaattcccgacctcagatgatccacccgcctcggtctcccaaagtgttgggattccaggtgtgagccaccacgcccggccaacagtattttctaataaccagtatatttccatatacatgtgtacatgggtattgtgattgttatcaggaaaaaatatattaaatggctgataggagaccatgggacgtattttctttctgcttttaaaaattattcaggccgggtgcggtggctcatgcctgtaatcccagccctttgggaggccaacgtggacagatcacctgaggtcaggagttcgggacaagcctggccaacatggtgaaaccctgtctctactaaaaacacaaaaattagccaggtgtggtggcaggcgcctgtaatcccaattagtgggttggctgaggcaggagaatcacttgaacccaggaggtggtggttgcagtgagctgagatcgtgccactgcaccccagcccgggtgacagagtgagactccatctcaaaaaaataaaaaataaaaaataaaaaataaattattcaatcttctttatttttattgttttaatgactaaagttatttttagtagaaacagggtcttgctatgttacccacgctagtcttgaactcattggcttaagcagtcttcctgccttagcctcccaaagtgctgggattacaggcatgcctggctcctccaccttcttaaaataagcatttgttttataattttttccaagtgtgtatcaagataaggaaatcaggaagtgtaatattcttatagaaatggccaaggcctcccccttcacctgtgcctcagatgctacccaatcccgccttctctgtccctccagaaggcaccctttgcttaggcctccctctcttcctgaaccacctctggaagtttcttattggcctatgaatgcattcttatttcttcttatcaaataaagccttccctttaatttatggcacatttatgctttggaatccactctcaggaataatcagtatgtagcatattacacgtcaggcggcaacattctttttttttttttttttttttgaggcagggtcccgctctgtcatccaggctagagtacggtggtgcaatcatagttcactgcagcctcaacttcctggggtcaagccatcttcccacctcagtctcccaagtagctggaaccacatgtgtgcactaccacacccagctagtttttttttgtagagacagggccttgccttgttgcccaggctggtctccaagtcctgggctcaagtgatgctcctgccttggccttccaaagtgctaggattacaggtgtgagctaccatgcctggtccaacattcttcatttggtaaatggctaaacttagtgcagagtatgagcctgattttgtttaaaaaaaaaatgtgtgtgggtgtgtatatgtatctttgagtgtatataaaaagactgaaagagggctgggcacggtggctcatgcctgtaatcccagcactttgggaggctgagggaggcggatcacttaagtcaggagtttgagaccagactgggtgaaaccccatctctactaaaaaatgcaaaaattagctgggcatggtggcgggcacctgtaatcccagcaaccagggaggctgaggcaggagaatcacttgaacccaggaggtggaggttgcagtgacccgagatgcagtgagactccacctcaaaaaaaaaaaaaaaaaaaaaaaaaaagactaaggatatatatcaaaacccttatggcagactgttatttgtaattgtattttatttgtcgtgcttatatgtgttgcccaagtttctatggtgaacggtatgcatcactttcagcatgagaaaataactcctaataaacgtgattcttaaaggactctccctgtgtacatcctttccaaggagccccgatgtaccctgcttccctcacagccaagctcctctaggacagtcctccctagggggttacggccttgctcctttgatggccctgccacagcccaggggctctttcctgggcactgtggaccagaccctcctagaatccctctccttcctcaaccccagactctcaggcaccccatctctatcctggacaccgagcttgtcaccaaggcccagctgcagctgctgtgctgagtgtgcacagccacctctgggaaggctgtgctccccctgaggacctgggtcctcagtctctgactctggggggattcggggccacctacccaccttctggctctactcaacaccagcagcccctcaacatcaagccctccagctcacagcacctcagctctgcagctccaaagccaccccagcctggaactggggactcagggagactccctcgctccaactccagctggtcccagcactgcctctgctgtcatctctggaatctggtctcttgtccccagcccactgctgccagggtcagccctggtactttctacctgggttgctgcaacagcttccctctgggggccgcctgttttcagccttgggcccatctgccctccacgttgctgctgggcaagctgaggccccaccacacccccagcaccttcctgtgcctccaggccttccctgccagtacaccccctccagcccctgaagccctgtcagcagctcagagcctttccccggagtcaccctgcttcccgaggggacccacgttgctgctgggcaagctgaggccccgccacaaccccagcgccttcctctgcctccagactccctcctagcacaccctctccagctcctgcagtcccaccagcagctcagagcctcttccctgagtcaccctgcttcccgaggggacctccacccagggaatgcccttccaggctcctctcactgattcaccttcactcctcagaggtcaggtgctggctgatagaggcaatgaggtgagcccctcacctcagggcgctcagcccctgggaaggtgatgaaaaagggcaccccaaggggtgtcccaaactaataggtggatgactcccgtgggaagatccagtctcccctccccatggccaattaaatttggggtgtttttctttacttttttgaggcaaggtcttgctttgttgctcaggctggagtgcagtggcacaatcacagctcactgcagccttgacctcctgggctcaagcaatcctcctccttcagcttcctgaggaggttttaattaaataaattaaaagctaattaaaaacatttttttttttttttggtagagatggggtctcactgttttccaggctgatcttgaactcctggactcaagcaatcctcttgcttcagcctctggagtagctgggactacaggtgcttgctactatgcccagctattttttttattttttatttttattttgtagtgatggggtgtgtttcctaggctgatcttgaactcctgggctcaagcaatcctcctgcctcggcctcccaaagtgctgagattataggtatgagccattgcactcagcctggattgtattaaggaaactagcttagtcactaaactgcacttctacttctgattggacaagattggcactttgtaaaccaggttttctgggatgctggtttccttgaagatgtttctagctggtattatcatataggataggctgagttatgctgcattaacaagcaaaacccaaaaggtttatttctcattcgtgctgtatgtccagcaaatgtggctgggcttcattttaagtcgtcctcactcccagacctagcctgtaggtgccccaccattgggggatcgctgattgccatggcatggttagagaaatatggttcttaaaggttctcatccagagtggcacatctctcttctgctcacaatgtgttgaccaaagcaagtcacatggatgggagcagggagatgcaatcctctcttgtgcccaaaagaggagcacaagaaatactgttgaatagcattgccagaggccacagaaaaatctccaaaaccaaaaatctctatgacccaataaattcaagaaatactgggttaaagagaaggaaaccagtatatgcactgtgagacttctcagtctttattatatacattgtaaccatcacagacagcctttaatatatacatcttaactatcacagacagccttcagtatattatatacattataaccatcacagacagccttcaatatattatatacattataaccatcacagacagcctttaatatatacattataactatcacagcctttaatatattatgtacattgtaactatcacagcctttaatatattatatacattgtaactaccaccgcctttaatatagtatacccattgtaactatcacagcctttaatatacacattgttaactaccacagacagcctttaatatattatatacattgtaactatcacagatagcctttaatatatacattgtaactatcgcagacagccctttgtttgagggtaaagcatgtgactttgttttcctcctgttccctctgccatcctccagggcctcacacacaataggctctcaaatattgcaaccatcttgcactctgcagggcaacctcatggcttatcctttacaagtggcatactcagaagaataacgaggaagaaagtgtaagaaaagagggctgtttgggcaaagaaaatctggttgattcgatgtttacagagtgccaggccccatgcttgatctcatgaggtcctcagaccttatgccttaggctatcatttccattttacagataaggaattgaggctcagggaactgagagtgagggaagggcaggggcctggctggtgctgggttctgtctctttccaggcaagaagggacatgtggacttgaatatttagctcagggatgttggaggcctagggtgctgctatttaaatgg

SEQ ID NO: 3 - DNA, 2222 bp, Homo sapiens
gtagagagcgcttgcgggcgccgggcggagctgctgcggatcaggacccgagccgattcccgatcccgacccagatcctaacccgcgcccccgccccgccgccgccgccatgtacgacgcagagcgcggctggagcttgtccttcgcgggctgcggcttcctgggcttctaccacgtcggggcgacccgctgcctgagcgagcacgccccgcacctcctccgcgacgcgcgcatgttgttcggcgcttcggccggggcgttgcactgcgtcggcgtcctctccgagcagactctgcaggtcctctcagatcttgtgcggaaggccaggagtcggaacattggcatcttccatccatccttcaacttaagcaagttcctccgacagggtctctgcaaatgcctcccggccaatgtccaccagctcatctccggcaaaatatgcatctctcttaccagagtgtctgatggggaaaacgttctggtgtctgactttcggtccaaagacgaagtcgtggatgccttggtatgttcctgcttcatgcctttctacagtggccttatccctccttccttcagaggcgtgcgatatgtggatggaggagtgagtgacaacgtacccttcattgatgccaaaacaaccatcaccgtgtcccccttctatggggagtacgacatctgccctaaagtcaagtccacgaactttcttcatgtggacatcaccaagctcagtctacgcctctgcacagggaacctctaccttctctcgagagcttttgtccccccggatctcaaggtgctgggagagatatgccttcgaggatatttggatgcattcaggttcttggaagagaagggcatctgcaacaggccccagccaggcctgaagtcatcctcagaagggatggatcctgaggtcgccatgcccagctgggcaaacatgagtctggattcttccccggagtcggctgccttggctgtgaggctggagggagatgagctgctagaccacctgcgtctcagcatcctgccctgggatgagagcatcctggacaccctctcgcccaggctcgctacagcactgagtgaagaaatgaaagacaaaggtggatacatgagcaagatttgcaacttgctacccattaggataatgtcttatgtaatgctgccctgtaccctgcctgtggaatctgccattgcgattgtccagagactggtgacatggcttccagatatgcccgacgatgtcctgtggttgcagtgggtgacctcacaggtgttcactcgagtgctgatgtgtctgctccccgcctccaggtcccaaatgccagtgagcagccaacaggcctccccatgcacacctgagcaggactggccctgctggactccctgctcccccgagggctgtccagcagagaccaaagcagaggccaccccgcggtccatcctcaggtccagcctgaacttcttcttgggcaataaagtacctgctggtgctgaggggctctccacctttcccagtttttcactagagaagagtctgtgagtcacttgaggaggcgagtctagcagattctttcagaggtgctaaagtttcccatctttgtgcagctacctccgcattgctgtgtagtgacccctgcctgtgacgtggaggatcccagcctctgagctgagttggttttatgaaaagctaggaagcaacctttcgcctgtgcagcggtccagcacttaactctaatacatcagcatgcgttaattcagctggttgggaaatgacaccaggaagcccagtgcagagggtcccttactgactgtttcgtggccctattaatggtcagactgttccagcatgaggttcttagaatgacaggtgtttggatgggtgggggccttgtgatggggggtaggctggcccatgtgtgatcttgtggggtggagggaagagaatagcatgatcccacttccccatgctgtgggaaggggtgcagttcgtccccaagaacgacactgcctgtcaggtggtctgcaaagatgataaccttgactactaaaaacgtctccatggcgggggtaacaagatgataatctacttaattttagaacacctttttcacctaactaaaataatgtttaaagagttttgtataaaaatgtaaggaagcgttgttacctgttgaattttgtattatgtgaatcagtgagatgttagtagaataagcctt

SEQ ID NO: 4 - DNA, 886 bp, Homo sapiens
  misc_feature (595)..(595): n is a, c, g, or t
  misc_feature (621)..(621): n is a, c, g, or t
ctgcccgggctcccgtgcggagtgggtgccccctaggccggggagcgggggatccccaggggtcgcggggccctggaggagcgggcatcggacgcggacacggcggggtgcatcccgagggccccctccgaggcagatgcttcctgcgggggcgctgttcctgggcccgggaagggggcgttggaaccccgagcggtccgggccgaagcctgggactctcagcagactctgcaggtcctctcagatcttgtgcggaaggccaggagtcggaacattggcatcttccatccatccttcaacttaagcaagttcctccgacagggtctcggcaaatgcctcccggccaatgtccaccagctcatctccggcaaaatatgcatctctcttaccagagtgtctgatggggaaaacgttctggtgtctgactttcggtccaaagacgaagtcgtggatgccttggtatgttcctgcttcatccccttctacagtggccttatccctccttccttcagaggcgtgcgatatgtggatggaggagtgagtgacaacgtaccccttcattgatgccaaaacaaccatcaccgtgtcccccttnctatgggagtacgacatctgccctanagtcaagtccacgaactttcttcatgtggacatcaccaagctcagtctacgcctctgcacagggaaacctctaccttctctcgagagcttttgtccccccggatctcaaggcatctgcaacaggccccagcccaggccctgaagtcctcctcaaaaggggatgggatcctggaggtcgcccttgccccggctgggccaaacattgagttctggatttcttccccccgagtccgcctgcccttggcctgtgaacgcttgaaagggc

SEQ ID NO: 5 - DNA, 423 bp, Homo sapiens
tttcaacccacgatcacggctcactgcacctccatctccctggttcaagcgattctcctgcctccgcctcccgagtagctgggattacaggcatgtgctaccaagccctgctatggggtccccttatctgaaggcggtttttctttccagctcgacctcttgtgacccttagtttaacaagggccgaagttaaagagtttctgcgcctggacccctaatgaagcaatcagatttctcatctccagtcaggtgtgggtccaagcccactagacaagtttgctcttcccagagcacatttctgccttcaagtcatcctggcttgtcagggctgggggagttctgctgtagaaatattagagtggaaggaaaaagatgtgttgggagctatttttctttaatactaaaagttggttgatgaatttg

SEQ ID NO: 6 - DNA, 568 bp, Homo sapiens
actgaggcagggtagagagcgcttgcgggcgccgggcggagctgctgcggatcaggacccgagccgattcccgatcccgacccagatcctaacccgcgcccccgccccgccgccgccgccatgtacgacgcagagcagcggctggagccttgtccttcgcgggcctgcggcttcctgggcttctaccacgtcggggcgacccgctgcctgagcgagcacgccccgcacctcctccgcgacgcgcgcatgttgttcggcgcttcggccggggcgttgcactgcgtcggcgtcctctccggtatcccgctggactctgcaggtcctctcagatcttgtgcggaaggccaggagtcggaacattggcatcttccatccatccttcaacttaagcaagttcctccgacagggtctcggcaaatgcctcccggccaatgtccaccagctcatctccggcaaaatatgcatctctcttaccagagtgtctgatggggaaaacgttctggtgtctgactttcggtccaaagacgaagtcgtggatgccttggtactttcctgcttcatccccttc

SEQ ID NO: 7-10 - skipped (4 entries)

SEQ ID NO: 11 - DNA, 21 bp, Artificial sequence
  Primer
ccttggtatgttcctgcttca

SEQ ID NO: 12 - DNA, 21 bp, Artificial sequence
  Primer
gttgtcactcactcctccatc

SEQ ID NO: 13 - DNA, 25 bp, Artificial sequence
  Probe
tggccttatccctccttccttcaga

SEQ ID NO: 14 - DNA, 18 bp, Artificial sequence
  Primer
tgaggctggagggagatg

SEQ ID NO: 15 - DNA, 22 bp, Artificial sequence
  Primer
gctcatgtatccacctttgtct

SEQ ID NO: 16 - DNA, 24 bp, Artificial sequence
  Probe
ctagaccacctgcgtctcagcatc

SEQ ID NO: 17 - DNA, 16 bp, Artificial sequence
  Synthetic oligonucleotide
ggcattcccagcgcga

SEQ ID NO: 18 - DNA, 16 bp, Artificial sequence
  Synthetic oligonucleotide
tcctgatccgcagcag

SEQ ID NO: 19 - DNA, 16 bp, Artificial sequence
  Synthetic oligonucleotide
ggctcgggtcctgatc

SEQ ID NO: 20 - DNA, 16 bp, Artificial sequence
  Synthetic oligonucleotide
gttaggatctgggtcg

SEQ ID NO: 21 - DNA, 16 bp, Artificial sequence
  Synthetic oligonucleotide
gtacatggcggcggcg

SEQ ID NO: 22 - DNA, 16 bp, Artificial sequence
  Synthetic oligonucleotide
tccagccgcgctctgc

SEQ ID NO: 23 - DNA, 16 bp, Artificial sequence
  Synthetic oligonucleotide
gcgaaggacaagctcc

SEQ ID NO: 24 - DNA, 16 bp, Artificial sequence
  Synthetic oligonucleotide
cgcgaaggacaagctc

SEQ ID NO: 25 - DNA, 16 bp, Artificial sequence
  Synthetic oligonucleotide
gcggaggaggtgcggg

SEQ ID NO: 26 - DNA, 16 bp, Artificial sequence
  Synthetic oligonucleotide
cgcggaggaggtgcgg

SEQ ID NO: 27 - DNA, 16 bp, Artificial sequence
  Synthetic oligonucleotide
tcgcggaggaggtgcg

SEQ ID NO: 28 - DNA, 16 bp, Artificial sequence
  Synthetic oligonucleotide
gaacaacatgcgcgcg

SEQ ID NO: 29 - DNA, 16 bp, Artificial sequence
  Synthetic oligonucleotide
cgaacaacatgcgcgc

SEQ ID NO: 30 - DNA, 16 bp, Artificial sequence
  Synthetic oligonucleotide
ccgaacaacatgcgcg

SEQ ID NO: 31 - DNA, 16 bp, Artificial sequence
  Synthetic oligonucleotide
gccgaacaacatgcgc

SEQ ID NO: 32 - DNA, 16 bp, Artificial sequence
  Synthetic oligonucleotide
cgccgaacaacatgcg

SEQ ID NO: 33 - DNA, 16 bp, Artificial sequence
  Synthetic oligonucleotide
gccgacgcagtgcaac

SEQ ID NO: 34 - DNA, 16 bp, Artificial sequence
  Synthetic oligonucleotide
cgccgacgcagtgcaa

SEQ ID NO: 35 - DNA, 16 bp, Artificial sequence
  Synthetic oligonucleotide
gggataccggagagga

SEQ ID NO: 36 - DNA, 16 bp, Artificial sequence
  Synthetic oligonucleotide
tctgagaggacctgca

SEQ ID NO: 37 - DNA, 16 bp, Artificial sequence
  Synthetic oligonucleotide
caagatctgagaggac

SEQ ID NO: 38 - DNA, 16 bp, Artificial sequence
  Synthetic oligonucleotide
gccaatgttccgactc

SEQ ID NO: 39 - DNA, 16 bp, Artificial sequence
  Synthetic oligonucleotide
gaagatgccaatgttc

SEQ ID NO: 40 - DNA, 16 bp, Artificial sequence
  Synthetic oligonucleotide
ttaagttgaaggatgg

SEQ ID NO: 41 - DNA, 16 bp, Artificial sequence
  Synthetic oligonucleotide
tgcttaagttgaagga

SEQ ID NO: 42 - DNA, 16 bp, Artificial sequence
  Synthetic oligonucleotide
tggacattggccggga

SEQ ID NO: 43 - DNA, 16 bp, Artificial sequence
  Synthetic oligonucleotide
gtggacattggccggg

SEQ ID NO: 44 - DNA, 16 bp, Artificial sequence
  Synthetic oligonucleotide
agctggtggacattgg

SEQ ID NO: 45 - DNA, 16 bp, Artificial sequence
  Synthetic oligonucleotide
catcagacactctggt

SEQ ID NO: 46 - DNA, 16 bp, Artificial sequence
  Synthetic oligonucleotide
ccccatcagacactct

SEQ ID NO: 47 - DNA, 16 bp, Artificial sequence
  Synthetic oligonucleotide
agtcagacaccagaac

SEQ ID NO: 48 - DNA, 16 bp, Artificial sequence
  Synthetic oligonucleotide
tggcatcaatgaaggg

SEQ ID NO: 49 - DNA, 16 bp, Artificial sequence
  Synthetic oligonucleotide
tgttttggcatcaatg

SEQ ID NO: 50 - DNA, 16 bp, Artificial sequence
  Synthetic oligonucleotide
tttagggcagatgtcg

SEQ ID NO: 51 - DNA, 16 bp, Artificial sequence
  Synthetic oligonucleotide
ctttagggcagatgtc

SEQ ID NO: 52 - DNA, 16 bp, Artificial sequence
  Synthetic oligonucleotide
gtagactgagcttggt

SEQ ID NO: 53 - DNA, 16 bp, Artificial sequence
  Synthetic oligonucleotide
aggtagaggttccctg

SEQ ID NO: 54 - DNA, 16 bp, Artificial sequence
  Synthetic oligonucleotide
gggacaaaagctctcg

SEQ ID NO: 55 - DNA, 16 bp, Artificial sequence
  Synthetic oligonucleotide
ggcatatctctcccag

SEQ ID NO: 56 - DNA, 16 bp, Artificial sequence
  Synthetic oligonucleotide
aggcatatctctccca

SEQ ID NO: 57 - DNA, 16 bp, Artificial sequence
  Synthetic oligonucleotide
aaatatcctcgaaggc

SEQ ID NO: 58 - DNA, 16 bp, Artificial sequence
  Synthetic oligonucleotide
ccaaatatcctcgaag

SEQ ID NO: 59 - DNA, 16 bp, Artificial sequence
  Synthetic oligonucleotide
tccaaatatcctcgaa

SEQ ID NO: 60 - DNA, 16 bp, Artificial sequence
  Synthetic oligonucleotide
atgcatccaaatatcc

SEQ ID NO: 61 - DNA, 16 bp, Artificial sequence
  Synthetic oligonucleotide
ttgcagatgcccttct

SEQ ID NO: 62 - DNA, 16 bp, Artificial sequence
  Synthetic oligonucleotide
atccatcccttctgag

SEQ ID NO: 63 - DNA, 16 bp, Artificial sequence
  Synthetic oligonucleotide
gggcatggcgacctca

SEQ ID NO: 64 - DNA, 16 bp, Artificial sequence
  Synthetic oligonucleotide
actcatgtttgcccag

SEQ ID NO: 65 - DNA, 16 bp, Artificial sequence
  Synthetic oligonucleotide
ggtctagcagctcatc

SEQ ID NO: 66 - DNA, 16 bp, Artificial sequence
  Synthetic oligonucleotide
cgcaggtggtctagca

SEQ ID NO: 67 - DNA, 16 bp, Artificial sequence
  Synthetic oligonucleotide
acgcaggtggtctagc

SEQ ID NO: 68 - DNA, 16 bp, Artificial sequence
  Synthetic oligonucleotide
tgagacgcaggtggtc

SEQ ID NO: 69 - DNA, 16 bp, Artificial sequence
  Synthetic oligonucleotide
ggatgctgagacgcag

SEQ ID NO: 70 - DNA, 16 bp, Artificial sequence
  Synthetic oligonucleotide
gtatccacctttgtct

SEQ ID NO: 71 - DNA, 16 bp, Artificial sequence
  Synthetic oligonucleotide
gctcatgtatccacct

SEQ ID NO: 72 - DNA, 16 bp, Artificial sequence
  Synthetic oligonucleotide
gcaaatcttgctcatg

SEQ ID NO: 73 - DNA, 16 bp, Artificial sequence
  Synthetic oligonucleotide
tgcaaatcttgctcat

SEQ ID NO: 74 - DNA, 16 bp, Artificial sequence
  Synthetic oligonucleotide
ttgcaaatcttgctca

SEQ ID NO: 75 - DNA, 16 bp, Artificial sequence
  Synthetic oligonucleotide
agcaagttgcaaatct

SEQ ID NO: 76 - DNA, 16 bp, Artificial sequence
  Synthetic oligonucleotide
gggtagcaagttgcaa

SEQ ID NO: 77 - DNA, 16 bp, Artificial sequence
  Synthetic oligonucleotide
cctaatgggtagcaag

SEQ ID NO: 78 - DNA, 16 bp, Artificial sequence
  Synthetic oligonucleotide
tcctaatgggtagcaa

SEQ ID NO: 79 - DNA, 16 bp, Artificial sequence
  Synthetic oligonucleotide
atcctaatgggtagca

SEQ ID NO: 80 - DNA, 16 bp, Artificial sequence
  Synthetic oligonucleotide
cattatcctaatgggt

SEQ ID NO: 81 - DNA, 16 bp, Artificial sequence
  Synthetic oligonucleotide
acattatcctaatggg

SEQ ID NO: 82 - DNA, 16 bp, Artificial sequence
  Synthetic oligonucleotide
gacattatcctaatgg

SEQ ID NO: 83 - DNA, 16 bp, Artificial sequence
  Synthetic oligonucleotide
tacataagacattatc

SEQ ID NO: 84 - DNA, 16 bp, Artificial sequence
  Synthetic oligonucleotide
gcattacataagacat

SEQ ID NO: 85 - DNA, 16 bp, Artificial sequence
  Synthetic oligonucleotide
ccacaggcagggtaca

SEQ ID NO: 86 - DNA, 16 bp, Artificial sequence
  Synthetic oligonucleotide
tccacaggcagggtac

SEQ ID NO: 87 - DNA, 16 bp, Artificial sequence
  Synthetic oligonucleotide
ggcagattccacaggc

SEQ ID NO: 88 - DNA, 16 bp, Artificial sequence
  Synthetic oligonucleotide
cgcaatggcagattcc

SEQ ID NO: 89 - DNA, 16 bp, Artificial sequence
  Synthetic oligonucleotide
gacaatcgcaatggca

SEQ ID NO: 90 - DNA, 16 bp, Artificial sequence
  Synthetic oligonucleotide
ggacaatcgcaatggc

SEQ ID NO: 91 - DNA, 16 bp, Artificial sequence
  Synthetic oligonucleotide
tggacaatcgcaatgg

SEQ ID NO: 92 - DNA, 16 bp, Artificial sequence
  Synthetic oligonucleotide
agccatgtcaccagtc

SEQ ID NO: 93 - DNA, 16 bp, Artificial sequence
  Synthetic oligonucleotide
tggaagccatgtcacc

SEQ ID NO: 94 - DNA, 16 bp, Artificial sequence
  Synthetic oligonucleotide
ctggaagccatgtcac

SEQ ID NO: 95 - DNA, 16 bp, Artificial sequence
  Synthetic oligonucleotide
ggcatatctggaagcc

SEQ ID NO: 96 - DNA, 16 bp, Artificial sequence
  Synthetic oligonucleotide
gggcatatctggaagc

SEQ ID NO: 97 - DNA, 16 bp, Artificial sequence
  Synthetic oligonucleotide
agcactcgagtgaaca

SEQ ID NO: 98 - DNA, 16 bp, Artificial sequence
  Synthetic oligonucleotide
gcatttgggacctgga

SEQ ID NO: 99 - DNA, 16 bp, Artificial sequence
  Synthetic oligonucleotide
ggcatttgggacctgg

SEQ ID NO: 100 - DNA, 16 bp, Artificial sequence
  Synthetic oligonucleotide
tggcatttgggacctg

SEQ ID NO: 101 - DNA, 16 bp, Artificial sequence
  Synthetic oligonucleotide
gctcactggcatttgg

SEQ ID NO: 102 - DNA, 16 bp, Artificial sequence
  Synthetic oligonucleotide
gttcaggctggacctg

SEQ ID NO: 103 - DNA, 16 bp, Artificial sequence
  Synthetic oligonucleotide
aggtactttattgccc

SEQ ID NO: 104 - DNA, 16 bp, Artificial sequence
  Synthetic oligonucleotide
agcaggtactttattg

SEQ ID NO: 105 - DNA, 16 bp, Artificial sequence
  Synthetic oligonucleotide
aactttagcacctctg

SEQ ID NO: 106 - DNA, 16 bp, Artificial sequence
  Synthetic oligonucleotide
gaaactttagcacctc

SEQ ID NO: 107 - DNA, 16 bp, Artificial sequence
  Synthetic oligonucleotide
ggaaactttagcacct

SEQ ID NO: 108 - DNA, 16 bp, Artificial sequence
  Synthetic oligonucleotide
ctgcacaaagatggga

SEQ ID NO: 109 - DNA, 16 bp, Artificial sequence
  Synthetic oligonucleotide
agctgcacaaagatgg

SEQ ID NO: 110 - DNA, 16 bp, Artificial sequence
  Synthetic oligonucleotide
agcaatgcggaggtag

SEQ ID NO: 111 - DNA, 16 bp, Artificial sequence
  Synthetic oligonucleotide
accaactcagctcaga

SEQ ID NO: 112 - DNA, 16 bp, Artificial sequence
  Synthetic oligonucleotide
aaccaactcagctcag

SEQ ID NO: 113 - DNA, 16 bp, Artificial sequence
  Synthetic oligonucleotide
tcctagcttttcataa

SEQ ID NO: 114 - DNA, 16 bp, Artificial sequence
  Synthetic oligonucleotide
tgctggaccgctgcac

SEQ ID NO: 115 - DNA, 16 bp, Artificial sequence
  Synthetic oligonucleotide
gagttaagtgctggac

SEQ ID NO: 116 - DNA, 16 bp, Artificial sequence
  Synthetic oligonucleotide
gtattagagttaagtg

SEQ ID NO: 117 - DNA, 16 bp, Artificial sequence
  Synthetic oligonucleotide
tgtattagagttaagt

SEQ ID NO: 118 - DNA, 16 bp, Artificial sequence
  Synthetic oligonucleotide
tgatgtattagagtta

SEQ ID NO: 119 - DNA, 16 bp, Artificial sequence
  Synthetic oligonucleotide
gctgatgtattagagt

SEQ ID NO: 120 - DNA, 16 bp, Artificial sequence
  Synthetic oligonucleotide
tgaattaacgcatgct

SEQ ID NO: 121 - DNA, 16 bp, Artificial sequence
  Synthetic oligonucleotide
ctgaattaacgcatgc

SEQ ID NO: 122 - DNA, 16 bp, Artificial sequence
  Synthetic oligonucleotide
agtaagggaccctctg

SEQ ID NO: 123 - DNA, 16 bp, Artificial sequence
  Synthetic oligonucleotide
cagtaagggaccctct

SEQ ID NO: 124 - DNA, 16 bp, Artificial sequence
  Synthetic oligonucleotide
tcagtaagggaccctc

SEQ ID NO: 125 - DNA, 16 bp, Artificial sequence
  Synthetic oligonucleotide
agtcagtaagggaccc

SEQ ID NO: 126 - DNA, 16 bp, Artificial sequence
  Synthetic oligonucleotide
attaatagggccacga

SEQ ID NO: 127 - DNA, 16 bp, Artificial sequence
  Synthetic oligonucleotide
ccattaatagggccac

SEQ ID NO: 128 - DNA, 16 bp, Artificial sequence
  Synthetic oligonucleotide
accattaatagggcca

SEQ ID NO: 129 - DNA, 16 bp, Artificial sequence
  Synthetic oligonucleotide
gaacagtctgaccatt

SEQ ID NO: 130 - DNA, 16 bp, Artificial sequence
  Synthetic oligonucleotide
tggaacagtctgacca

SEQ ID NO: 131 - DNA, 16 bp, Artificial sequence
  Synthetic oligonucleotide
ctggaacagtctgacc

SEQ ID NO: 132 - DNA, 16 bp, Artificial sequence
  Synthetic oligonucleotide
tgctggaacagtctga

SEQ ID NO: 133 - DNA, 16 bp, Artificial sequence
  Synthetic oligonucleotide
cctcatgctggaacag

SEQ ID NO: 134 - DNA, 16 bp, Artificial sequence
  Synthetic oligonucleotide
tcattctaagaacctc

SEQ ID NO: 135 - DNA, 16 bp, Artificial sequence
  Synthetic oligonucleotide
acccatccaaacacct

SEQ ID NO: 136 - DNA, 16 bp, Artificial sequence
  Synthetic oligonucleotide
acacatgggccagcct

SEQ ID NO: 137 - DNA, 16 bp, Artificial sequence
  Synthetic oligonucleotide
caagatcacacatggg

SEQ ID NO: 138 - DNA, 16 bp, Artificial sequence
  Synthetic oligonucleotide
gggacgaactgcaccc

SEQ ID NO: 139 - DNA, 16 bp, Artificial sequence
  Synthetic oligonucleotide
tatcatctttgcagac

SEQ ID NO: 140 - DNA, 16 bp, Artificial sequence
  Synthetic oligonucleotide
gtttttagtagtcaag

SEQ ID NO: 141 - DNA, 16 bp, Artificial sequence
  Synthetic oligonucleotide
cgtttttagtagtcaa

SEQ ID NO: 142 - DNA, 16 bp, Artificial sequence
  Synthetic oligonucleotide
tatcatcttgttaccc

SEQ ID NO: 143 - DNA, 16 bp, Artificial sequence
  Synthetic oligonucleotide
gattatcatcttgtta

SEQ ID NO: 144 - DNA, 16 bp, Artificial sequence
  Synthetic oligonucleotide
tagattatcatcttgt

SEQ ID NO: 145 - DNA, 16 bp, Artificial sequence
  Synthetic oligonucleotide
gtagattatcatcttg

SEQ ID NO: 146 - DNA, 16 bp, Artificial sequence
  Synthetic oligonucleotide
agtagattatcatctt

SEQ ID NO: 147 - DNA, 16 bp, Artificial sequence
  Synthetic oligonucleotide
gtgaaaaaggtgttct

SEQ ID NO: 148 - DNA, 16 bp, Artificial sequence
  Synthetic oligonucleotide
tagttaggtgaaaaag

SEQ ID NO: 149 - DNA, 16 bp, Artificial sequence
  Synthetic oligonucleotide
ttattttagttaggtg

SEQ ID NO: 150 - DNA, 16 bp, Artificial sequence
  Synthetic oligonucleotide
cattattttagttagg

SEQ ID NO: 151 - DNA, 16 bp, Artificial sequence
  Synthetic oligonucleotide
ctactaacatctcact

SEQ ID NO: 152 - DNA, 16 bp, Artificial sequence
  Synthetic oligonucleotide
tctactaacatctcac

SEQ ID NO: 153 - DNA, 16 bp, Artificial sequence
  Synthetic oligonucleotide
ttattctactaacatc

SEQ ID NO: 154 - DNA, 16 bp, Artificial sequence
  Synthetic oligonucleotide
gcttattctactaaca

SEQ ID NO: 155 - DNA, 16 bp, Artificial sequence
  Synthetic oligonucleotide
ggcttattctactaac

SEQ ID NO: 156 - DNA, 16 bp, Artificial sequence
  Synthetic oligonucleotide
ggtgaatgccctgcac

SEQ ID NO: 157 - DNA, 16 bp, Artificial sequence
  Synthetic oligonucleotide
ttcaagttgtgtgctc

SEQ ID NO: 158 - DNA, 16 bp, Artificial sequence
  Synthetic oligonucleotide
gggagaaactcactga

SEQ ID NO: 159 - DNA, 16 bp, Artificial sequence
  Synthetic oligonucleotide
tgctacttgccccagc

SEQ ID NO: 160 - DNA, 16 bp, Artificial sequence
  Synthetic oligonucleotide
cacaatgctacttgcc

SEQ ID NO: 161 - DNA, 16 bp, Artificial sequence
  Synthetic oligonucleotide
cccaatggcagggctt

SEQ ID NO: 162 - DNA, 16 bp, Artificial sequence
  Synthetic oligonucleotide
tgctcctacccaatgg

SEQ ID NO: 163 - DNA, 16 bp, Artificial sequence
  Synthetic oligonucleotide
gacttttattgttgct

SEQ ID NO: 164 - DNA, 16 bp, Artificial sequence
  Synthetic oligonucleotide
ttctataccagagtga

SEQ ID NO: 165 - DNA, 16 bp, Artificial sequence
  Synthetic oligonucleotide
tttctataccagagtg

SEQ ID NO: 166 - DNA, 16 bp, Artificial sequence
  Synthetic oligonucleotide
gtagatggccttaatg

SEQ ID NO: 167 - DNA, 16 bp, Artificial sequence
  Synthetic oligonucleotide
tacatccacgacttcg

SEQ ID NO: 168 - DNA, 16 bp, Artificial sequence
  Synthetic oligonucleotide
ttacatccacgacttc

SEQ ID NO: 169 - DNA, 16 bp, Artificial sequence
  Synthetic oligonucleotide
ggaacattcagggttt

SEQ ID NO: 170 - DNA, 16 bp, Artificial sequence
  Synthetic oligonucleotide
attacttgggtgcagg

SEQ ID NO: 171 - DNA, 16 bp, Artificial sequence
  Synthetic oligonucleotide
gcagattacttgggtg

SEQ ID NO: 172 - DNA, 16 bp, Artificial sequence
  Synthetic oligonucleotide
tgcaggacaggttcct

SEQ ID NO: 173 - DNA, 16 bp, Artificial sequence
  Synthetic oligonucleotide
cacactgggtcaccac

SEQ ID NO: 174 - DNA, 16 bp, Artificial sequence
  Synthetic oligonucleotide
agtcacactgggtcac

SEQ ID NO: 175 - DNA, 16 bp, Artificial sequence
  Synthetic oligonucleotide
ggtatatgttcccagg

SEQ ID NO: 176 - DNA, 16 bp, Artificial sequence
  Synthetic oligonucleotide
tataaccacagcctgg

SEQ ID NO: 177 - DNA, 16 bp, Artificial sequence
  Synthetic oligonucleotide
ctgactatataaccac

SEQ ID NO: 178 - DNA, 16 bp, Artificial sequence
  Synthetic oligonucleotide
atcttagtggctgggt

SEQ ID NO: 179 - DNA, 16 bp, Artificial sequence
  Synthetic oligonucleotide
cttactatggtagagt

SEQ ID NO: 180 - DNA, 16 bp, Artificial sequence
  Synthetic oligonucleotide
tcttactatggtagag

SEQ ID NO: 181 - DNA, 16 bp, Artificial sequence
  Synthetic oligonucleotide
tgcattgcatagcctt

SEQ ID NO: 182 - DNA, 16 bp, Artificial sequence
  Synthetic oligonucleotide
ttgcattgcatagcct

SEQ ID NO: 183 - DNA, 16 bp, Artificial sequence
  Synthetic oligonucleotide
tgcttataaagcacac

SEQ ID NO: 184 - DNA, 16 bp, Artificial sequence
  Synthetic oligonucleotide
ggaataagcctccacc

SEQ ID NO: 185 - DNA, 16 bp, Artificial sequence
  Synthetic oligonucleotide
gaaatctgattgcttc

SEQ ID NO: 186 - DNA, 16 bp, Artificial sequence
  Synthetic oligonucleotide
tacttatctgctcact

SEQ ID NO: 187 - DNA, 16 bp, Artificial sequence
  Synthetic oligonucleotide
tctcttagtgtcccca

SEQ ID NO: 188 - DNA, 16 bp, Artificial sequence
  Synthetic oligonucleotide
atctcttagtgtcccc

SEQ ID NO: 189 - DNA, 16 bp, Artificial sequence
  Synthetic oligonucleotide
tcacattcatgcttgc

SEQ ID NO: 190 - DNA, 16 bp, Artificial sequence
  Synthetic oligonucleotide
gataacctcacattca

SEQ ID NO: 191 - DNA, 16 bp, Artificial sequence
  Synthetic oligonucleotide
gagctaggtgcttcac

SEQ ID NO: 192 - DNA, 16 bp, Artificial sequence
  Synthetic oligonucleotide
ataacaactgaaccac

SEQ ID NO: 193 - DNA, 16 bp, Artificial sequence
  Synthetic oligonucleotide
gttattagccaaatgc

SEQ ID NO: 194 - DNA, 16 bp, Artificial sequence
  Synthetic oligonucleotide
ggagacttggcaaggt

SEQ ID NO: 195 - DNA, 16 bp, Artificial sequence
  Synthetic oligonucleotide
attcatgacagccctt

SEQ ID NO: 196 - DNA, 16 bp, Artificial sequence
  Synthetic oligonucleotide
atcgatttttcagagt

SEQ ID NO: 197 - DNA, 16 bp, Artificial sequence
  Synthetic oligonucleotide
acaaacatcgattttt

SEQ ID NO: 198 - DNA, 16 bp, Artificial sequence
  Synthetic oligonucleotide
ctctttaatgacctcg

SEQ ID NO: 199 - DNA, 16 bp, Artificial sequence
  Synthetic oligonucleotide
gtcagaggcactcaca

SEQ ID NO: 200 - DNA, 16 bp, Artificial sequence
  Synthetic oligonucleotide
agctattatctcccac

SEQ ID NO: 201 - DNA, 16 bp, Artificial sequence
  Synthetic oligonucleotide
agtttctgggcttgca

SEQ ID NO: 202 - DNA, 16 bp, Artificial sequence
  Synthetic oligonucleotide
ggcaatcacaagagac

SEQ ID NO: 203 - DNA, 16 bp, Artificial sequence
  Synthetic oligonucleotide
agaggaagcccaatca

SEQ ID NO: 204 - DNA, 16 bp, Artificial sequence
  Synthetic oligonucleotide
cagaggaagcccaatc

SEQ ID NO: 205 - DNA, 16 bp, Artificial sequence
  Synthetic oligonucleotide
tagaaattgcagtgcc

SEQ ID NO: 206 - DNA, 16 bp, Artificial sequence
  Synthetic oligonucleotide
tcctatccatatattg

SEQ ID NO: 207 - DNA, 16 bp, Artificial sequence
  Synthetic oligonucleotide
gcaattctagacatgg

SEQ ID NO: 208 - DNA, 16 bp, Artificial sequence
  Synthetic oligonucleotide
aggacttacaccaaga

SEQ ID NO: 209 - DNA, 16 bp, Artificial sequence
  Synthetic oligonucleotide
ttcctaataagagccc

SEQ ID NO: 210 - DNA, 16 bp, Artificial sequence
  Synthetic oligonucleotide
gtcaaacatcttccta

SEQ ID NO: 211 - DNA, 16 bp, Artificial sequence
  Synthetic oligonucleotide
aaaactgtaggatagg

SEQ ID NO: 212 - DNA, 16 bp, Artificial sequence
  Synthetic oligonucleotide
gttacatccataaaac

SEQ ID NO: 213 - DNA, 16 bp, Artificial sequence
  Synthetic oligonucleotide
agagaatgttacatcc

SEQ ID NO: 214 - DNA, 16 bp, Artificial sequence
  Synthetic oligonucleotide
aaagattaatcagggc

SEQ ID NO: 215 - DNA, 16 bp, Artificial sequence
  Synthetic oligonucleotide
gtatttacctggaggc

SEQ ID NO: 216 - DNA, 16 bp, Artificial sequence
  Synthetic oligonucleotide
ggcctatgattttcag

SEQ ID NO: 217 - DNA, 16 bp, Artificial sequence
  Synthetic oligonucleotide
atacttttggcaaggc

SEQ ID NO: 218 - DNA, 16 bp, Artificial sequence
  Synthetic oligonucleotide
aatacttttggcaagg

SEQ ID NO: 219 - DNA, 16 bp, Artificial sequence
  Synthetic oligonucleotide
tgcttacatccacgac

SEQ ID NO: 220 - DNA, 16 bp, Artificial sequence
  Synthetic oligonucleotide
catcatgttggtctcg

SEQ ID NO: 221 - DNA, 16 bp, Artificial sequence
  Synthetic oligonucleotide
gattacttgggtgcag

SEQ ID NO: 222 - DNA, 16 bp, Artificial sequence
  Synthetic oligonucleotide
cagattacttgggtgc

SEQ ID NO: 223 - DNA, 16 bp, Artificial sequence
  Synthetic oligonucleotide
tttaatggtgttttgg

SEQ ID NO: 224 - DNA, 16 bp, Artificial sequence
  Synthetic oligonucleotide
tcaaatgccggtattc

SEQ ID NO: 225 - DNA, 16 bp, Artificial sequence
  Synthetic oligonucleotide
gtgaacttcaacttcc

SEQ ID NO: 226 - DNA, 16 bp, Artificial sequence
  Synthetic oligonucleotide
ctatataaccacagcc

SEQ ID NO: 227 - DNA, 16 bp, Artificial sequence
  Synthetic oligonucleotide
gactatataaccacag

SEQ ID NO: 228 - DNA, 16 bp, Artificial sequence
  Synthetic oligonucleotide
aatcatcttagtggct

SEQ ID NO: 229 - DNA, 16 bp, Artificial sequence
  Synthetic oligonucleotide
tactatggtagagtgg

SEQ ID NO: 230 - DNA, 16 bp, Artificial sequence
  Synthetic oligonucleotide
gtacatggtctgcaaa

SEQ ID NO: 231 - DNA, 16 bp, Artificial sequence
  Synthetic oligonucleotide
tgtacatggtctgcaa

SEQ ID NO: 232 - DNA, 16 bp, Artificial sequence
  Synthetic oligonucleotide
gcatgcattgcattgc

SEQ ID NO: 233 - DNA, 16 bp, Artificial sequence
  Synthetic oligonucleotide
accaatcctgttagac

SEQ ID NO: 234 - DNA, 16 bp, Artificial sequence
  Synthetic oligonucleotide
ggagacaccaagcacc

SEQ ID NO: 235 - DNA, 16 bp, Artificial sequence
  Synthetic oligonucleotide
gcactaagtgttagaa

SEQ ID NO: 236 - DNA, 16 bp, Artificial sequence
  Synthetic oligonucleotide
gcttacttatctgctc

SEQ ID NO: 237 - DNA, 16 bp, Artificial sequence
  Synthetic oligonucleotide
ggagatccatcctgca

SEQ ID NO: 238 - DNA, 16 bp, Artificial sequence
  Synthetic oligonucleotide
catctcttagtgtccc

SEQ ID NO: 239 - DNA, 16 bp, Artificial sequence
  Synthetic oligonucleotide
tcctaatgtcctcaac

SEQ ID NO: 240 - DNA, 16 bp, Artificial sequence
  Synthetic oligonucleotide
aagataacctcacatt

SEQ ID NO: 241 - DNA, 16 bp, Artificial sequence
  Synthetic oligonucleotide
caagataacctcacat

SEQ ID NO: 242 - DNA, 16 bp, Artificial sequence
  Synthetic oligonucleotide
tataacaactgaacca

SEQ ID NO: 243 - DNA, 16 bp, Artificial sequence
  Synthetic oligonucleotide
gctttaaagcaggaca

SEQ ID NO: 244 - DNA, 16 bp, Artificial sequence
  Synthetic oligonucleotide
aaaattgtgggtttag

SEQ ID NO: 245 - DNA, 16 bp, Artificial sequence
  Synthetic oligonucleotide
atcatttggaccatag

SEQ ID NO: 246 - DNA, 16 bp, Artificial sequence
  Synthetic oligonucleotide
acatcgatttttcaga

SEQ ID NO: 247 - DNA, 16 bp, Artificial sequence
  Synthetic oligonucleotide
caaacatcgatttttc

SEQ ID NO: 248 - DNA, 16 bp, Artificial sequence
  Synthetic oligonucleotide
gctttacaagctggtc

SEQ ID NO: 249 - DNA, 16 bp, Artificial sequence
  Synthetic oligonucleotide
atctatgttctcctag

SEQ ID NO: 250 - DNA, 16 bp, Artificial sequence
  Synthetic oligonucleotide
acctaaaatgctcacc

SEQ ID NO: 251 - DNA, 16 bp, Artificial sequence
  Synthetic oligonucleotide
ccagactacatgccac

SEQ ID NO: 252 - DNA, 16 bp, Artificial sequence
  Synthetic oligonucleotide
tctactaggcatctct

SEQ ID NO: 253 - DNA, 16 bp, Artificial sequence
  Synthetic oligonucleotide
ttctactaggcatctc

SEQ ID NO: 254 - DNA, 16 bp, Artificial sequence
  Synthetic oligonucleotide
tcagaggaagcccaat

SEQ ID NO: 255 - DNA, 16 bp, Artificial sequence
  Synthetic oligonucleotide
gaaattgcagtgccct

SEQ ID NO: 256 - DNA, 16 bp, Artificial sequence
  Synthetic oligonucleotide
gccaacctatcactga

SEQ ID NO: 257 - DNA, 16 bp, Artificial sequence
  Synthetic oligonucleotide
agacatggccaaccta

SEQ ID NO: 258 - DNA, 16 bp, Artificial sequence
  Synthetic oligonucleotide
tgaaataaggacttac

SEQ ID NO: 259 - DNA, 16 bp, Artificial sequence
  Synthetic oligonucleotide
cctaataagagcccca

SEQ ID NO: 260 - DNA, 16 bp, Artificial sequence
  Synthetic oligonucleotide
gaaatctgtcagagca

SEQ ID NO: 261 - DNA, 16 bp, Artificial sequence
  Synthetic oligonucleotide
tgtaggataggactag

SEQ ID NO: 262 - DNA, 16 bp, Artificial sequence
  Synthetic oligonucleotide
gaatgttacatccata

SEQ ID NO: 263 - DNA, 16 bp, Artificial sequence
  Synthetic oligonucleotide
gagaatgttacatcca

SEQ ID NO: 264 - DNA, 16 bp, Artificial sequence
  Synthetic oligonucleotide
gtgataaatctgcaag

SEQ ID NO: 265 - DNA, 16 bp, Artificial sequence
  Synthetic oligonucleotide
agattaatcagggcca

SEQ ID NO: 266 - DNA, 16 bp, Artificial sequence
  Synthetic oligonucleotide
aagattaatcagggcc

SEQ ID NO: 267 - DNA, 16 bp, Artificial sequence
  Synthetic oligonucleotide
ggtcacatgtgagccc

SEQ ID NO: 268 - DNA, 16 bp, Artificial sequence
  Synthetic oligonucleotide
cacttctggttcaaga

SEQ ID NO: 269 - DNA, 16 bp, Artificial sequence
  Synthetic oligonucleotide
ccaatctgatgacttc

SEQ ID NO: 270 - DNA, 16 bp, Artificial sequence
  Synthetic oligonucleotide
aagtatttacctggag

SEQ ID NO: 271 - DNA, 16 bp, Artificial sequence
  Synthetic oligonucleotide
cactcaaagagactca

SEQ ID NO: 272 - DNA, 16 bp, Artificial sequence
  Synthetic oligonucleotide
gcctatgattttcagg

SEQ ID NO: 273 - DNA, 16 bp, Artificial sequence
  Synthetic oligonucleotide
cactactgccctcttc

SEQ ID NO: 274 - DNA, 16 bp, Artificial sequence
  Synthetic oligonucleotide
tgctgggctgatgtca

SEQ ID NO: 275 - DNA, 16 bp, Artificial sequence
  Synthetic oligonucleotide
ggcatttgggacctga

SEQ ID NO: 276 - DNA, 16 bp, Artificial sequence
  Synthetic oligonucleotide
gcccccctcggaccat

SEQ ID NO: 277 - DNA, 16 bp, Artificial sequence
  Synthetic oligonucleotide
cctcagtgtctcggcc

SEQ ID NO: 278 - DNA, 16 bp, Artificial sequence
  Synthetic oligonucleotide
aatcggctcgggtcct

SEQ ID NO: 279 - DNA, 16 bp, Artificial sequence
  Synthetic oligonucleotide
gacaagctccagccgc

SEQ ID NO: 280 - DNA, 16 bp, Artificial sequence
  Synthetic oligonucleotide
cgctcaggcagcgggt

SEQ ID NO: 281 - DNA, 16 bp, Artificial sequence
  Synthetic oligonucleotide
ctccagcgggataccg

SEQ ID NO: 282 - DNA, 16 bp, Artificial sequence
  Synthetic oligonucleotide
ggccttccgcacaaga

SEQ ID NO: 283 - DNA, 16 bp, Artificial sequence
  Synthetic oligonucleotide
tggatggaagatgcca

SEQ ID NO: 284 - DNA, 16 bp, Artificial sequence
  Synthetic oligonucleotide
gagaccctgtcggagg

SEQ ID NO: 285 - DNA, 16 bp, Artificial sequence
  Synthetic oligonucleotide
gatgagctggtggaca

SEQ ID NO: 286 - DNA, 16 bp, Artificial sequence
  Synthetic oligonucleotide
gagagatgcctatttt

SEQ ID NO: 287 - DNA, 16 bp, Artificial sequence
  Synthetic oligonucleotide
gaccgaaagtcagaca

SEQ ID NO: 288 - DNA, 16 bp, Artificial sequence
  Synthetic oligonucleotide
gttttggcatcaatga

SEQ ID NO: 289 - DNA, 16 bp, Artificial sequence
  Synthetic oligonucleotide
gacttgactttagggc

SEQ ID NO: 290 - DNA, 16 bp, Artificial sequence
  Synthetic oligonucleotide
cgagagaaggtagagg

SEQ ID NO: 291 - DNA, 16 bp, Artificial sequence
  Synthetic oligonucleotide
ctcgaaggcatatctc

SEQ ID NO: 292 - DNA, 16 bp, Artificial sequence
  Synthetic oligonucleotide
gggcctgttgcagatg

SEQ ID NO: 293 - DNA, 16 bp, Artificial sequence
  Synthetic oligonucleotide
ggcatggcgacctcag

SEQ ID NO: 294 - DNA, 16 bp, Artificial sequence
  Synthetic oligonucleotide
agccaaggcagccgac

SEQ ID NO: 295 - DNA, 16 bp, Artificial sequence
  Synthetic oligonucleotide
gcgagcctgggcgaga

SEQ ID NO: 296 - DNA, 16 bp, Artificial sequence
  Synthetic oligonucleotide
ctcatgtatccacctt

SEQ ID NO: 297 - DNA, 16 bp, Artificial sequence
  Synthetic oligonucleotide
gggcagcattacataa

SEQ ID NO: 298 - DNA, 16 bp, Artificial sequence
  Synthetic oligonucleotide
aagccatgtcaccagt

SEQ ID NO: 299 - DNA, 16 bp, Artificial sequence
  Synthetic oligonucleotide
actcgagtgaacacct

SEQ ID NO: 300 - DNA, 16 bp, Artificial sequence
  Synthetic oligonucleotide
gcctgttggctgctca

SEQ ID NO: 301 - DNA, 16 bp, Artificial sequence
  Synthetic oligonucleotide
ctgctggacagccctt

SEQ ID NO: 302 - DNA, 16 bp, Artificial sequence
  Synthetic oligonucleotide
ctttattgcccaagaa

SEQ ID NO: 303 - DNA, 16 bp, Artificial sequence
  Synthetic oligonucleotide
cagactcttctctagt

SEQ ID NO: 304 - DNA, 16 bp, Artificial sequence
  Synthetic oligonucleotide
aatctgctagactcgc

SEQ ID NO: 305 - DNA, 16 bp, Artificial sequence
  Synthetic oligonucleotide
cagcaatgcggaggta

SEQ ID NO: 306 - DNA, 16 bp, Artificial sequence
  Synthetic oligonucleotide
gaaaggttgcttccta

SEQ ID NO: 307 - DNA, 16 bp, Artificial sequence
  Synthetic oligonucleotide
gtgctggaccgctgca

SEQ ID NO: 308 - DNA, 16 bp, Artificial sequence
  Synthetic oligonucleotide
aacgcatgctgatgta

SEQ ID NO: 309 - DNA, 16 bp, Artificial sequence
  Synthetic oligonucleotide
gcttcctggtgtcatt

SEQ ID NO: 310 - DNA, 16 bp, Artificial sequence
  Synthetic oligonucleotide
gccacgaaacagtcag

SEQ ID NO: 311 - DNA, 16 bp, Artificial sequence
  Synthetic oligonucleotide
catgctggaacagtct

SEQ ID NO: 312 - DNA, 16 bp, Artificial sequence
  Synthetic oligonucleotide
aaggcccccacccatc

SEQ ID NO: 313 - DNA, 16 bp, Artificial sequence
  Synthetic oligonucleotide
tgggccagcctacccc

SEQ ID NO: 314 - DNA, 16 bp, Artificial sequence
  Synthetic oligonucleotide
ggaagtgggatcatgc

SEQ ID NO: 315 - DNA, 16 bp, Artificial sequence
  Synthetic oligonucleotide
gttatcatctttgcag

SEQ ID NO: 316 - DNA, 16 bp, Artificial sequence
  Synthetic oligonucleotide
cttgttacccccgcca

SEQ ID NO: 317 - DNA, 16 bp, Artificial sequence
  Synthetic oligonucleotide
tctcactgattcacat

SEQ ID NO: 318 - DNA, 16 bp, Artificial sequence
  Synthetic oligonucleotide
ccctgcacactagatt

SEQ ID NO: 319 - DNA, 16 bp, Artificial sequence
  Synthetic oligonucleotide
gaggcggaagctcctg

SEQ ID NO: 320 - DNA, 16 bp, Artificial sequence
  Synthetic oligonucleotide
caggttcaagttgtgt

SEQ ID NO: 321 - DNA, 16 bp, Artificial sequence
  Synthetic oligonucleotide
aaatgtacggaatctc

SEQ ID NO: 322 - DNA, 16 bp, Artificial sequence
  Synthetic oligonucleotide
gtgtaaacatttgtcc

SEQ ID NO: 323 - DNA, 16 bp, Artificial sequence
  Synthetic oligonucleotide
agctttggtgtagatg

SEQ ID NO: 324 - DNA, 16 bp, Artificial sequence
  Synthetic oligonucleotide
tactatgggagccaca

SEQ ID NO: 325 - DNA, 16 bp, Artificial sequence
  Synthetic oligonucleotide
tgaaattgtaactgcc

SEQ ID NO: 326 - DNA, 16 bp, Artificial sequence
  Synthetic oligonucleotide
tagatcggtgctgttc

SEQ ID NO: 327 - DNA, 16 bp, Artificial sequence
  Synthetic oligonucleotide
gttataggcgagagca

SEQ ID NO: 328 - DNA, 16 bp, Artificial sequence
  Synthetic oligonucleotide
tatataaccacagcct

SEQ ID NO: 329 - DNA, 16 bp, Artificial sequence
  Synthetic oligonucleotide
ataagagctgtctcct

SEQ ID NO: 330 - DNA, 16 bp, Artificial sequence
  Synthetic oligonucleotide
ctagtaaatgcttgtc

SEQ ID NO: 331 - DNA, 16 bp, Artificial sequence
  Synthetic oligonucleotide
ctaatatttctacagc

SEQ ID NO: 332 - DNA, 16 bp, Artificial sequence
  Synthetic oligonucleotide
ctcttagtgtccccat

SEQ ID NO: 333 - DNA, 16 bp, Artificial sequence
  Synthetic oligonucleotide
ttccatcacaaggcct

SEQ ID NO: 334 - DNA, 16 bp, Artificial sequence
  Synthetic oligonucleotide
tccataatgcacaaga

SEQ ID NO: 335 - DNA, 16 bp, Artificial sequence
  Synthetic oligonucleotide
tgtagctggtttgtgg

SEQ ID NO: 336 - DNA, 16 bp, Artificial sequence
  Synthetic oligonucleotide
aacagctacatcaggc

SEQ ID NO: 337 - DNA, 16 bp, Artificial sequence
  Synthetic oligonucleotide
ggcattgcacatagac

SEQ ID NO: 338 - DNA, 16 bp, Artificial sequence
  Synthetic oligonucleotide
gtaagcaatgcagcca

SEQ ID NO: 339 - DNA, 16 bp, Artificial sequence
  Synthetic oligonucleotide
ttagaaattgcagtgc

SEQ ID NO: 340 - DNA, 16 bp, Artificial sequence
  Synthetic oligonucleotide
aggtattaaactgcca

SEQ ID NO: 341 - DNA, 16 bp, Artificial sequence
  Synthetic oligonucleotide
gtcctaagagcactca

SEQ ID NO: 342 - DNA, 16 bp, Artificial sequence
  Synthetic oligonucleotide
gataaatctgcaagag

SEQ ID NO: 343 - DNA, 16 bp, Artificial sequence
  Synthetic oligonucleotide
gggacttacactgaaa

SEQ ID NO: 344 - DNA, 16 bp, Artificial sequence
  Synthetic oligonucleotide
gtcaacgcagactgct

SEQ ID NO: 345 - DNA, 16 bp, Artificial sequence
  Synthetic oligonucleotide
cgcccccctcggacca

SEQ ID NO: 346 - DNA, 16 bp, Artificial sequence
  Synthetic oligonucleotide
gcctcagtgtctcggc

SEQ ID NO: 347 - DNA, 16 bp, Artificial sequence
  Synthetic oligonucleotide
gaatcggctcgggtcc

SEQ ID NO: 348 - DNA, 16 bp, Artificial sequence
  Synthetic oligonucleotide
ggacaagctccagccg

SEQ ID NO: 349 - DNA, 16 bp, Artificial sequence
  Synthetic oligonucleotide
tcgctcaggcagcggg

SEQ ID NO: 350 - DNA, 16 bp, Artificial sequence
  Synthetic oligonucleotide
gctccagcgggatacc

SEQ ID NO: 351 - DNA, 16 bp, Artificial sequence
  Synthetic oligonucleotide
tggccttccgcacaag

SEQ ID NO: 352 - DNA, 16 bp, Artificial sequence
  Synthetic oligonucleotide
taagttgaaggatgga

SEQ ID NO: 353 - DNA, 16 bp, Artificial sequence
  Synthetic oligonucleotide
agagaccctgtcggag

SEQ ID NO: 354 - DNA, 16 bp, Artificial sequence
  Synthetic oligonucleotide
agatgagctggtggac

SEQ ID NO: 355 - DNA, 16 bp, Artificial sequence
  Synthetic oligonucleotide
aagagagatgcctatt

SEQ ID NO: 356 - DNA, 16 bp, Artificial sequence
  Synthetic oligonucleotide
ggaccgaaagtcagac

SEQ ID NO: 357 - DNA, 16 bp, Artificial sequence
  Synthetic oligonucleotide
gttgttttggcatcaa

SEQ ID NO: 358 - DNA, 16 bp, Artificial sequence
  Synthetic oligonucleotide
ggacttgactttaggg

SEQ ID NO: 359 - DNA, 16 bp, Artificial sequence
  Synthetic oligonucleotide
tcgagagaaggtagag

SEQ ID NO: 360 - DNA, 16 bp, Artificial sequence
  Synthetic oligonucleotide
cctcgaaggcatatct

SEQ ID NO: 361 - DNA, 16 bp, Artificial sequence
  Synthetic oligonucleotide
gatgacttcaggcctg

SEQ ID NO: 362 - DNA, 16 bp, Artificial sequence
  Synthetic oligonucleotide
tgggcatggcgacctc

SEQ ID NO: 363 - DNA, 16 bp, Artificial sequence
  Synthetic oligonucleotide
cagccaaggcagccga

SEQ ID NO: 364 - DNA, 16 bp, Artificial sequence
  Synthetic oligonucleotide
agcgagcctgggcgag

SEQ ID NO: 365 - DNA, 16 bp, Artificial sequence
  Synthetic oligonucleotide
tgctcatgtatccacc

SEQ ID NO: 366 - DNA, 16 bp, Artificial sequence
  Synthetic oligonucleotide
agggcagcattacata

SEQ ID NO: 367 - DNA, 16 bp, Artificial sequence
  Synthetic oligonucleotide
tatctggaagccatgt

SEQ ID NO: 368 - DNA, 16 bp, Artificial sequence
  Synthetic oligonucleotide
cactcgagtgaacacc

SEQ ID NO: 369 - DNA, 16 bp, Artificial sequence
  Synthetic oligonucleotide
ggcctgttggctgctc

SEQ ID NO: 370 - DNA, 16 bp, Artificial sequence
  Synthetic oligonucleotide
gtctctgctggacagc

SEQ ID NO: 371 - DNA, 16 bp, Artificial sequence
  Synthetic oligonucleotide
gtactttattgcccaa

SEQ ID NO: 372 - DNA, 16 bp, Artificial sequence
  Synthetic oligonucleotide
gactcacagactcttc

SEQ ID NO: 373 - DNA, 16 bp, Artificial sequence
  Synthetic oligonucleotide
gaatctgctagactcg

SEQ ID NO: 374 - DNA, 16 bp, Artificial sequence
  Synthetic oligonucleotide
acagcaatgcggaggt

SEQ ID NO: 375 - DNA, 16 bp, Artificial sequence
  Synthetic oligonucleotide
cgaaaggttgcttcct

SEQ ID NO: 376 - DNA, 16 bp, Artificial sequence
  Synthetic oligonucleotide
agtgctggaccgctgc

SEQ ID NO: 377 - DNA, 16 bp, Artificial sequence
  Synthetic oligonucleotide
taacgcatgctgatgt

SEQ ID NO: 378 - DNA, 16 bp, Artificial sequence
  Synthetic oligonucleotide
ggcttcctggtgtcat

SEQ ID NO: 379 - DNA, 16 bp, Artificial sequence
  Synthetic oligonucleotide
ggccacgaaacagtca

SEQ ID NO: 380 - DNA, 16 bp, Artificial sequence
  Synthetic oligonucleotide
tcatgctggaacagtc

SEQ ID NO: 381 - DNA, 16 bp, Artificial sequence
  Synthetic oligonucleotide
tcacaaggcccccacc

SEQ ID NO: 382 - DNA, 16 bp, Artificial sequence
  Synthetic oligonucleotide
atgggccagcctaccc

SEQ ID NO: 383 - DNA, 16 bp, Artificial sequence
  Synthetic oligonucleotide
cgaactgcaccccttc

SEQ ID NO: 384 - DNA, 16 bp, Artificial sequence
  Synthetic oligonucleotide
ggttatcatctttgca

SEQ ID NO: 385 - DNA, 16 bp, Artificial sequence
  Synthetic oligonucleotide
tcttgttacccccgcc

SEQ ID NO: 386 - DNA, 16 bp, Artificial sequence
  Synthetic oligonucleotide
atctcactgattcaca

SEQ ID NO: 387 - DNA, 16 bp, Artificial sequence
  Synthetic oligonucleotide
gccctgcacactagat

SEQ ID NO: 388 - DNA, 16 bp, Artificial sequence
  Synthetic oligonucleotide
ggaggcggaagctcct

SEQ ID NO: 389 - DNA, 16 bp, Artificial sequence
  Synthetic oligonucleotide
gccaggttcaagttgt

SEQ ID NO: 390 - DNA, 16 bp, Artificial sequence
  Synthetic oligonucleotide
caaatgtacggaatct

SEQ ID NO: 391 - DNA, 16 bp, Artificial sequence
  Synthetic oligonucleotide
tactttaggctcctgg

SEQ ID NO: 392 - DNA, 16 bp, Artificial sequence
  Synthetic oligonucleotide
agcattagagctttgg

SEQ ID NO: 393 - DNA, 16 bp, Artificial sequence
  Synthetic oligonucleotide
tctactatgggagcca

SEQ ID NO: 394 - DNA, 16 bp, Artificial sequence
  Synthetic oligonucleotide
ggacaggttccttgga

SEQ ID NO: 395 - DNA, 16 bp, Artificial sequence
  Synthetic oligonucleotide
ctagatcggtgctgtt

SEQ ID NO: 396 - DNA, 16 bp, Artificial sequence
  Synthetic oligonucleotide
agttataggcgagagc

SEQ ID NO: 397 - DNA, 16 bp, Artificial sequence
  Synthetic oligonucleotide
actatataaccacagc

SEQ ID NO: 398 - DNA, 16 bp, Artificial sequence
  Synthetic oligonucleotide
gacaataagagctgtc

SEQ ID NO: 399 - DNA, 16 bp, Artificial sequence
  Synthetic oligonucleotide
gctagtaaatgcttgt

SEQ ID NO: 400 - DNA, 16 bp, Artificial sequence
  Synthetic oligonucleotide
ccaacttttagtatta

SEQ ID NO: 401 - DNA, 16 bp, Artificial sequence
  Synthetic oligonucleotide
agccatctcttagtgt

SEQ ID NO: 402 - DNA, 16 bp, Artificial sequence
  Synthetic oligonucleotide
tctgatgtcgaagagg

SEQ ID NO: 403 - DNA, 16 bp, Artificial sequence
  Synthetic oligonucleotide
tcccatgtggcagtac

SEQ ID NO: 404 - DNA, 16 bp, Artificial sequence
  Synthetic oligonucleotide
tccaaatgcccaactc

SEQ ID NO: 405 - DNA, 16 bp, Artificial sequence
  Synthetic oligonucleotide
gcaaataatgtgcaca

SEQ ID NO: 406 - DNA, 16 bp, Artificial sequence
  Synthetic oligonucleotide
gggcattgcacataga

SEQ ID NO: 407 - DNA, 16 bp, Artificial sequence
  Synthetic oligonucleotide
gtagtaagcaatgcag

SEQ ID NO: 408 - DNA, 16 bp, Artificial sequence
  Synthetic oligonucleotide
cttagaaattgcagtg

SEQ ID NO: 409 - DNA, 16 bp, Artificial sequence
  Synthetic oligonucleotide
attttaacagctcagg

SEQ ID NO: 410 - DNA, 16 bp, Artificial sequence
  Synthetic oligonucleotide
tatgacatttcagagt

SEQ ID NO: 411 - DNA, 16 bp, Artificial sequence
  Synthetic oligonucleotide
agtacaagcgcagcct

SEQ ID NO: 412 - DNA, 16 bp, Artificial sequence
  Synthetic oligonucleotide
acaaggacaagcccac

SEQ ID NO: 413 - DNA, 16 bp, Artificial sequence
  Synthetic oligonucleotide
gaagtagcggcatccc

SEQ ID NO: 414 - DNA, 16 bp, Artificial sequence
  Synthetic oligonucleotide
ccgcccccctcggacc

SEQ ID NO: 415 - DNA, 16 bp, Artificial sequence
  Synthetic oligonucleotide
tgcctcagtgtctcgg

SEQ ID NO: 416 - DNA, 16 bp, Artificial sequence
  Synthetic oligonucleotide
ggaatcggctcgggtc

SEQ ID NO: 417 - DNA, 16 bp, Artificial sequence
  Synthetic oligonucleotide
aaggacaagctccagc

SEQ ID NO: 418 - DNA, 16 bp, Artificial sequence
  Synthetic oligonucleotide
ctcgctcaggcagcgg

SEQ ID NO: 419 - DNA, 16 bp, Artificial sequence
  Synthetic oligonucleotide
tgctccagcgggatac

SEQ ID NO: 420 - DNA, 16 bp, Artificial sequence
  Synthetic oligonucleotide
ctggccttccgcacaa

SEQ ID NO: 421 - DNA, 16 bp, Artificial sequence
  Synthetic oligonucleotide
cttaagttgaaggatg

SEQ ID NO: 422 - DNA, 16 bp, Artificial sequence
  Synthetic oligonucleotide
cagagaccctgtcgga

SEQ ID NO: 423 - DNA, 16 bp, Artificial sequence
  Synthetic oligonucleotide
cggagatgagctggtg

SEQ ID NO: 424 - DNA, 16 bp, Artificial sequence
  Synthetic oligonucleotide
taagagagatgcctat

SEQ ID NO: 425 - DNA, 16 bp, Artificial sequence
  Synthetic oligonucleotide
tggaccgaaagtcaga

SEQ ID NO: 426 - DNA, 16 bp, Artificial sequence
  Synthetic oligonucleotide
ggttgttttggcatca

SEQ ID NO: 427 - DNA, 16 bp, Artificial sequence
  Synthetic oligonucleotide
tggacttgactttagg

SEQ ID NO: 428 - DNA, 16 bp, Artificial sequence
  Synthetic oligonucleotide
ctcgagagaaggtaga

SEQ ID NO: 429 - DNA, 16 bp, Artificial sequence
  Synthetic oligonucleotide
tcctcgaaggcatatc

SEQ ID NO: 430 - DNA, 16 bp, Artificial sequence
  Synthetic oligonucleotide
ggatgacttcaggcct

SEQ ID NO: 431 - DNA, 16 bp, Artificial sequence
  Synthetic oligonucleotide
ctgggcatggcgacct

SEQ ID NO: 432 - DNA, 16 bp, Artificial sequence
  Synthetic oligonucleotide
acagccaaggcagccg

SEQ ID NO: 433 - DNA, 16 bp, Artificial sequence
  Synthetic oligonucleotide
tagcgagcctgggcga

SEQ ID NO: 434 - DNA, 16 bp, Artificial sequence
  Synthetic oligonucleotide
caagttgcaaatcttg

SEQ ID NO: 435 - DNA, 16 bp, Artificial sequence
  Synthetic oligonucleotide
cagggcagcattacat

SEQ ID NO: 436 - DNA, 16 bp, Artificial sequence
  Synthetic oligonucleotide
tcgggcatatctggaa

SEQ ID NO: 437 - DNA, 16 bp, Artificial sequence
  Synthetic oligonucleotide
gcactcgagtgaacac

SEQ ID NO: 438 - DNA, 16 bp, Artificial sequence
  Synthetic oligonucleotide
aggcctgttggctgct

SEQ ID NO: 439 - DNA, 16 bp, Artificial sequence
  Synthetic oligonucleotide
ttggtctctgctggac

SEQ ID NO: 440 - DNA, 16 bp, Artificial sequence
  Synthetic oligonucleotide
ggtactttattgccca

SEQ ID NO: 441 - DNA, 16 bp, Artificial sequence
  Synthetic oligonucleotide
gtgactcacagactct

SEQ ID NO: 442 - DNA, 16 bp, Artificial sequence
  Synthetic oligonucleotide
agaatctgctagactc

SEQ ID NO: 443 - DNA, 16 bp, Artificial sequence
  Synthetic oligonucleotide
cacagcaatgcggagg

SEQ ID NO: 444 - DNA, 16 bp, Artificial sequence
  Synthetic oligonucleotide
gcgaaaggttgcttcc

SEQ ID NO: 445 - DNA, 16 bp, Artificial sequence
  Synthetic oligonucleotide
aagtgctggaccgctg

SEQ ID NO: 446 - DNA, 16 bp, Artificial sequence
  Synthetic oligonucleotide
ttaacgcatgctgatg

SEQ ID NO: 447 - DNA, 16 bp, Artificial sequence
  Synthetic oligonucleotide
gggcttcctggtgtca

SEQ ID NO: 448 - DNA, 16 bp, Artificial sequence
  Synthetic oligonucleotide
gggccacgaaacagtc

SEQ ID NO: 449 - DNA, 16 bp, Artificial sequence
  Synthetic oligonucleotide
ctcatgctggaacagt

SEQ ID NO: 450 - DNA, 16 bp, Artificial sequence
  Synthetic oligonucleotide
atcacaaggcccccac

SEQ ID NO: 451 - DNA, 16 bp, Artificial sequence
  Synthetic oligonucleotide
catgggccagcctacc

SEQ ID NO: 452 - DNA, 16 bp, Artificial sequence
  Synthetic oligonucleotide
acgaactgcacccctt

SEQ ID NO: 453 - DNA, 16 bp, Artificial sequence
  Synthetic oligonucleotide
aggttatcatctttgc

SEQ ID NO: 454 - DNA, 16 bp, Artificial sequence
  Synthetic oligonucleotide
atcttgttacccccgc

SEQ ID NO: 455 - DNA, 16 bp, Artificial sequence
  Synthetic oligonucleotide
catctcactgattcac

SEQ ID NO: 456 - DNA, 16 bp, Artificial sequence
  Synthetic oligonucleotide
tgccctgcacactaga

SEQ ID NO: 457 - DNA, 16 bp, Artificial sequence
  Synthetic oligonucleotide
gaggaggcggaagctc

SEQ ID NO: 458 - DNA, 16 bp, Artificial sequence
  Synthetic oligonucleotide
agccaggttcaagttg

SEQ ID NO: 459 - DNA, 16 bp, Artificial sequence
  Synthetic oligonucleotide
tcaaatgtacggaatc

SEQ ID NO: 460 - DNA, 16 bp, Artificial sequence
  Synthetic oligonucleotide
gtactttaggctcctg

SEQ ID NO: 461 - DNA, 16 bp, Artificial sequence
  Synthetic oligonucleotide
acatatcagcattaga

SEQ ID NO: 462 - DNA, 16 bp, Artificial sequence
  Synthetic oligonucleotide
gtctactatgggagcc

SEQ ID NO: 463 - DNA, 16 bp, Artificial sequence
  Synthetic oligonucleotide
gaagatgcatagagga

SEQ ID NO: 464 - DNA, 16 bp, Artificial sequence
  Synthetic oligonucleotide
tcacactgggtcacca

SEQ ID NO: 465 - DNA, 16 bp, Artificial sequence
  Synthetic oligonucleotide
ggcaatcagggaggca

SEQ ID NO: 466 - DNA, 16 bp, Artificial sequence
  Synthetic oligonucleotide
tgactatataaccaca

SEQ ID NO: 467 - DNA, 16 bp, Artificial sequence
  Synthetic oligonucleotide
cccaattgccactagg

SEQ ID NO: 468 - DNA, 16 bp, Artificial sequence
  Synthetic oligonucleotide
tctttaccaagaccgc

SEQ ID NO: 469 - DNA, 16 bp, Artificial sequence
  Synthetic oligonucleotide
gacaaattcatcaacc

SEQ ID NO: 470 - DNA, 16 bp, Artificial sequence
  Synthetic oligonucleotide
ctgtatccaaaaggcc

SEQ ID NO: 471 - DNA, 16 bp, Artificial sequence
  Synthetic oligonucleotide
atacatagcagagcca

SEQ ID NO: 472 - DNA, 16 bp, Artificial sequence
  Synthetic oligonucleotide
caccctatcgctccca

SEQ ID NO: 473 - DNA, 16 bp, Artificial sequence
  Synthetic oligonucleotide
agttatgtctgactca

SEQ ID NO: 474 - DNA, 16 bp, Artificial sequence
  Synthetic oligonucleotide
aatataccccacagca

SEQ ID NO: 475 - DNA, 16 bp, Artificial sequence
  Synthetic oligonucleotide
gtgcatgtgtggcttg

SEQ ID NO: 476 - DNA, 16 bp, Artificial sequence
  Synthetic oligonucleotide
tgtagtaagcaatgca

SEQ ID NO: 477 - DNA, 16 bp, Artificial sequence
  Synthetic oligonucleotide
catatattgcggatga

SEQ ID NO: 478 - DNA, 16 bp, Artificial sequence
  Synthetic oligonucleotide
gttattttaacagctc

SEQ ID NO: 479 - DNA, 16 bp, Artificial sequence
  Synthetic oligonucleotide
ataaggacttacacca

SEQ ID NO: 480 - DNA, 16 bp, Artificial sequence
  Synthetic oligonucleotide
cagcatgcaaccaccc

SEQ ID NO: 481 - DNA, 16 bp, Artificial sequence
  Synthetic oligonucleotide
tgggatgctaggacaa

SEQ ID NO: 482 - DNA, 16 bp, Artificial sequence
  Synthetic oligonucleotide
ggaagtagcggcatcc

SEQ ID NO: 483 - DNA, 16 bp, Artificial sequence
  Synthetic oligonucleotide
cattcccagcgcgacg

SEQ ID NO: 484 - DNA, 16 bp, Artificial sequence
  Synthetic oligonucleotide
taccctgcctcagtgt

SEQ ID NO: 485 - DNA, 16 bp, Artificial sequence
  Synthetic oligonucleotide
tcgggaatcggctcgg

SEQ ID NO: 486 - DNA, 16 bp, Artificial sequence
  Synthetic oligonucleotide
cgaaggacaagctcca

SEQ ID NO: 487 - DNA, 16 bp, Artificial sequence
  Synthetic oligonucleotide
gctcgctcaggcagcg

SEQ ID NO: 488 - DNA, 16 bp, Artificial sequence
  Synthetic oligonucleotide
ctgctccagcgggata

SEQ ID NO: 489 - DNA, 16 bp, Artificial sequence
  Synthetic oligonucleotide
cctggccttccgcaca

SEQ ID NO: 490 - DNA, 16 bp, Artificial sequence
  Synthetic oligonucleotide
gcttaagttgaaggat

SEQ ID NO: 491 - DNA, 16 bp, Artificial sequence
  Synthetic oligonucleotide
gcagagaccctgtcgg

SEQ ID NO: 492 - DNA, 16 bp, Artificial sequence
  Synthetic oligonucleotide
ccggagatgagctggt

SEQ ID NO: 493 - DNA, 16 bp, Artificial sequence
  Synthetic oligonucleotide
gtaagagagatgccta

SEQ ID NO: 494 - DNA, 16 bp, Artificial sequence
  Synthetic oligonucleotide
ttggaccgaaagtcag

SEQ ID NO: 495 - DNA, 16 bp, Artificial sequence
  Synthetic oligonucleotide
tggttgttttggcatc

SEQ ID NO: 496 - DNA, 16 bp, Artificial sequence
  Synthetic oligonucleotide
gtggacttgactttag

SEQ ID NO: 497 - DNA, 16 bp, Artificial sequence
  Synthetic oligonucleotide
tctcgagagaaggtag

SEQ ID NO: 498 - DNA, 16 bp, Artificial sequence
  Synthetic oligonucleotide
atcctcgaaggcatat

SEQ ID NO: 499 - DNA, 16 bp, Artificial sequence
  Synthetic oligonucleotide
tgaggatgacttcagg

SEQ ID NO: 500 - DNA, 16 bp, Artificial sequence
  Synthetic oligonucleotide
gctgggcatggcgacc

SEQ ID NO: 501 - DNA, 16 bp, Artificial sequence
  Synthetic oligonucleotide
tagcagctcatctccc

SEQ ID NO: 502 - DNA, 16 bp, Artificial sequence
  Synthetic oligonucleotide
gtagcgagcctgggcg

SEQ ID NO: 503 - DNA, 16 bp, Artificial sequence
  Synthetic oligonucleotide
tagcaagttgcaaatc

SEQ ID NO: 504 - DNA, 16 bp, Artificial sequence
  Synthetic oligonucleotide
acagggcagcattaca

SEQ ID NO: 505 - DNA, 16 bp, Artificial sequence
  Synthetic oligonucleotide
cgtcgggcatatctgg

SEQ ID NO: 506 - DNA, 16 bp, Artificial sequence
  Synthetic oligonucleotide
cagcactcgagtgaac

SEQ ID NO: 507 - DNA, 16 bp, Artificial sequence
  Synthetic oligonucleotide
gaggcctgttggctgc

SEQ ID NO: 508 - DNA, 16 bp, Artificial sequence
  Synthetic oligonucleotide
gaggatggaccgcggg

SEQ ID NO: 509 - DNA, 16 bp, Artificial sequence
  Synthetic oligonucleotide
gcaggtactttattgc

SEQ ID NO: 510 - DNA, 16 bp, Artificial sequence
  Synthetic oligonucleotide
agtgactcacagactc

SEQ ID NO: 511 - DNA, 16 bp, Artificial sequence
  Synthetic oligonucleotide
aagaatctgctagact

SEQ ID NO: 512 - DNA, 16 bp, Artificial sequence
  Synthetic oligonucleotide
acacagcaatgcggag

SEQ ID NO: 513 - DNA, 16 bp, Artificial sequence
  Synthetic oligonucleotide
ggcgaaaggttgcttc

SEQ ID NO: 514 - DNA, 16 bp, Artificial sequence
  Synthetic oligonucleotide
taagtgctggaccgct

SEQ ID NO: 515 - DNA, 16 bp, Artificial sequence
  Synthetic oligonucleotide
attaacgcatgctgat

SEQ ID NO: 516 - DNA, 16 bp, Artificial sequence
  Synthetic oligonucleotide
tgggcttcctggtgtc

SEQ ID NO: 517 - DNA, 16 bp, Artificial sequence
  Synthetic oligonucleotide
agggccacgaaacagt

SEQ ID NO: 518 - DNA, 16 bp, Artificial sequence
  Synthetic oligonucleotide
acctcatgctggaaca

SEQ ID NO: 519 - DNA, 16 bp, Artificial sequence
  Synthetic oligonucleotide
catcacaaggccccca

SEQ ID NO: 520 - DNA, 16 bp, Artificial sequence
  Synthetic oligonucleotide
acatgggccagcctac

SEQ ID NO: 521 - DNA, 16 bp, Artificial sequence
  Synthetic oligonucleotide
gacgaactgcacccct

SEQ ID NO: 522 - DNA, 16 bp, Artificial sequence
  Synthetic oligonucleotide
tcaaggttatcatctt

SEQ ID NO: 523 - DNA, 16 bp, Artificial sequence
  Synthetic oligonucleotide
catcttgttacccccg

SEQ ID NO: 524 - DNA, 16 bp, Artificial sequence
  Synthetic oligonucleotide
ctaacatctcactgat

SEQ ID NO: 525 - DNA, 16 bp, Artificial sequence
  Synthetic oligonucleotide
atgccctgcacactag

SEQ ID NO: 526 - DNA, 16 bp, Artificial sequence
  Synthetic oligonucleotide
agaggaggcggaagct

SEQ ID NO: 527 - DNA, 16 bp, Artificial sequence
  Synthetic oligonucleotide
aagccaggttcaagtt

SEQ ID NO: 528 - DNA, 16 bp, Artificial sequence
  Synthetic oligonucleotide
attaggacaagattca

SEQ ID NO: 529 - DNA, 16 bp, Artificial sequence
  Synthetic oligonucleotide
tgtactttaggctcct

SEQ ID NO: 530 - DNA, 16 bp, Artificial sequence
  Synthetic oligonucleotide
aacatatcagcattag

SEQ ID NO: 531 - DNA, 16 bp, Artificial sequence
  Synthetic oligonucleotide
caaggatgccaccaac

SEQ ID NO: 532 - DNA, 16 bp, Artificial sequence
  Synthetic oligonucleotide
tcattatggaagatgc

SEQ ID NO: 533 - DNA, 16 bp, Artificial sequence
  Synthetic oligonucleotide
ttaacaaccctgtcag

SEQ ID NO: 534 - DNA, 16 bp, Artificial sequence
  Synthetic oligonucleotide
gtaactggtagctcct

SEQ ID NO: 535 - DNA, 16 bp, Artificial sequence
  Synthetic oligonucleotide
acccatactgcacccc

SEQ ID NO: 536 - DNA, 16 bp, Artificial sequence
  Synthetic oligonucleotide
gcctatcccaattgcc

SEQ ID NO: 537 - DNA, 16 bp, Artificial sequence
  Synthetic oligonucleotide
gtctttaccaagaccg

SEQ ID NO: 538 - DNA, 16 bp, Artificial sequence
  Synthetic oligonucleotide
aacgacaaattcatca

SEQ ID NO: 539 - DNA, 16 bp, Artificial sequence
  Synthetic oligonucleotide
tgcaatccccctgtat

SEQ ID NO: 540 - DNA, 16 bp, Artificial sequence
  Synthetic oligonucleotide
aatacatagcagagcc

SEQ ID NO: 541 - DNA, 16 bp, Artificial sequence
  Synthetic oligonucleotide
tgtcatggttgcctca

SEQ ID NO: 542 - DNA, 16 bp, Artificial sequence
  Synthetic oligonucleotide
ataaggagttatgtct

SEQ ID NO: 543 - DNA, 16 bp, Artificial sequence
  Synthetic oligonucleotide
gaatataccccacagc

SEQ ID NO: 544 - DNA, 16 bp, Artificial sequence
  Synthetic oligonucleotide
gttacaggtgcatgtg

SEQ ID NO: 545 - DNA, 16 bp, Artificial sequence
  Synthetic oligonucleotide
agtcatctggagtcac

SEQ ID NO: 546 - DNA, 16 bp, Artificial sequence
  Synthetic oligonucleotide
tcagacaacccactga

SEQ ID NO: 547 - DNA, 16 bp, Artificial sequence
  Synthetic oligonucleotide
aggaatctgaatccta

SEQ ID NO: 548 - DNA, 16 bp, Artificial sequence
  Synthetic oligonucleotide
gataatttcctagagc

SEQ ID NO: 549 - DNA, 16 bp, Artificial sequence
  Synthetic oligonucleotide
gaaataagtgctcagg

SEQ ID NO: 550 - DNA, 16 bp, Artificial sequence
  Synthetic oligonucleotide
ctccaatctgatgact

SEQ ID NO: 551 - DNA, 16 bp, Artificial sequence
  Synthetic oligonucleotide
gaattcaggaagtagc

SEQ ID NO: 552 - DNA, 16 bp, Artificial sequence
  Synthetic oligonucleotide
gcattcccagcgcgac

SEQ ID NO: 553 - DNA, 16 bp, Artificial sequence
  Synthetic oligonucleotide
gctctctaccctgcct

SEQ ID NO: 554 - DNA, 16 bp, Artificial sequence
  Synthetic oligonucleotide
atcgggaatcggctcg

SEQ ID NO: 555 - DNA, 16 bp, Artificial sequence
  Synthetic oligonucleotide
ccgcgaaggacaagct

SEQ ID NO: 556 - DNA, 16 bp, Artificial sequence
  Synthetic oligonucleotide
tgctcgctcaggcagc

SEQ ID NO: 557 - DNA, 16 bp, Artificial sequence
  Synthetic oligonucleotide
tctgctccagcgggat

SEQ ID NO: 558 - DNA, 16 bp, Artificial sequence
  Synthetic oligonucleotide
ctcctggccttccgca

SEQ ID NO: 559 - DNA, 16 bp, Artificial sequence
  Synthetic oligonucleotide
ttgcttaagttgaagg

SEQ ID NO: 560 - DNA, 16 bp, Artificial sequence
  Synthetic oligonucleotide
tgcagagaccctgtcg

SEQ ID NO: 561 - DNA, 16 bp, Artificial sequence
  Synthetic oligonucleotide
gccggagatgagctgg

SEQ ID NO: 562 - DNA, 16 bp, Artificial sequence
  Synthetic oligonucleotide
ggtaagagagatgcct

SEQ ID NO: 563 - DNA, 16 bp, Artificial sequence
  Synthetic oligonucleotide
tttggaccgaaagtca

SEQ ID NO: 564 - DNA, 16 bp, Artificial sequence
  Synthetic oligonucleotide
atggttgttttggcat

SEQ ID NO: 565 - DNA, 16 bp, Artificial sequence
  Synthetic oligonucleotide
cgtggacttgacttta

SEQ ID NO: 566 - DNA, 16 bp, Artificial sequence
  Synthetic oligonucleotide
ctctcgagagaaggta

SEQ ID NO: 567 - DNA, 16 bp, Artificial sequence
  Synthetic oligonucleotide
tatcctcgaaggcata

SEQ ID NO: 568 - DNA, 16 bp, Artificial sequence
  Synthetic oligonucleotide
ttctgaggatgacttc

SEQ ID NO: 569 - DNA, 16 bp, Artificial sequence
  Synthetic oligonucleotide
ttgcccagctgggcat

SEQ ID NO: 570 - DNA, 16 bp, Artificial sequence
  Synthetic oligonucleotide
ctagcagctcatctcc

SEQ ID NO: 571 - DNA, 16 bp, Artificial sequence
  Synthetic oligonucleotide
tgtagcgagcctgggc

SEQ ID NO: 572 - DNA, 16 bp, Artificial sequence
  Synthetic oligonucleotide
gtagcaagttgcaaat

SEQ ID NO: 573 - DNA, 16 bp, Artificial sequence
  Synthetic oligonucleotide
tacagggcagcattac

SEQ ID NO: 574 - DNA, 16 bp, Artificial sequence
  Synthetic oligonucleotide
caaccacaggacatcg

SEQ ID NO: 575 - DNA, 16 bp, Artificial sequence
  Synthetic oligonucleotide
tcagcactcgagtgaa

SEQ ID NO: 576 - DNA, 16 bp, Artificial sequence
  Synthetic oligonucleotide
ggaggcctgttggctg

SEQ ID NO: 577 - DNA, 16 bp, Artificial sequence
  Synthetic oligonucleotide
tgaggatggaccgcgg

SEQ ID NO: 578 - DNA, 16 bp, Artificial sequence
  Synthetic oligonucleotide
accagcaggtacttta

SEQ ID NO: 579 - DNA, 16 bp, Artificial sequence
  Synthetic oligonucleotide
aagtgactcacagact

SEQ ID NO: 580 - DNA, 16 bp, Artificial sequence
  Synthetic oligonucleotide
aaagaatctgctagac

SEQ ID NO: 581 - DNA, 16 bp, Artificial sequence
  Synthetic oligonucleotide
tacacagcaatgcgga

SEQ ID NO: 582 - DNA, 16 bp, Artificial sequence
  Synthetic oligonucleotide
aggcgaaaggttgctt

SEQ ID NO: 583 - DNA, 16 bp, Artificial sequence
  Synthetic oligonucleotide
ttaagtgctggaccgc

SEQ ID NO: 584 - DNA, 16 bp, Artificial sequence
  Synthetic oligonucleotide
aattaacgcatgctga

SEQ ID NO: 585 - DNA, 16 bp, Artificial sequence
  Synthetic oligonucleotide
ggaccctctgcactgg

SEQ ID NO: 586 - DNA, 16 bp, Artificial sequence
  Synthetic oligonucleotide
tagggccacgaaacag

SEQ ID NO: 587 - DNA, 16 bp, Artificial sequence
  Synthetic oligonucleotide
aacctcatgctggaac

SEQ ID NO: 588 - DNA, 16 bp, Artificial sequence
  Synthetic oligonucleotide
ccatcacaaggccccc

SEQ ID NO: 589 - DNA, 16 bp, Artificial sequence
  Synthetic oligonucleotide
cacatgggccagccta

SEQ ID NO: 590 - DNA, 16 bp, Artificial sequence
  Synthetic oligonucleotide
ggacgaactgcacccc

SEQ ID NO: 591 - DNA, 16 bp, Artificial sequence
  Synthetic oligonucleotide
gtcaaggttatcatct

SEQ ID NO: 592 - DNA, 16 bp, Artificial sequence
  Synthetic oligonucleotide
tcatcttgttaccccc

SEQ ID NO: 593 - DNA, 16 bp, Artificial sequence
  Synthetic oligonucleotide
tactaacatctcactg

SEQ ID NO: 594 - DNA, 16 bp, Artificial sequence
  Synthetic oligonucleotide
aatgccctgcacacta

SEQ ID NO: 595 - DNA, 16 bp, Artificial sequence
  Synthetic oligonucleotide
gagaggaggcggaagc

SEQ ID NO: 596 - DNA, 16 bp, Artificial sequence
  Synthetic oligonucleotide
taagccaggttcaagt

SEQ ID NO: 597 - DNA, 16 bp, Artificial sequence
  Synthetic oligonucleotide
tttcattaggacaaga

SEQ ID NO: 598 - DNA, 16 bp, Artificial sequence
  Synthetic oligonucleotide
gtgtactttaggctcc

SEQ ID NO: 599 - DNA, 16 bp, Artificial sequence
  Synthetic oligonucleotide
gaacatatcagcatta

SEQ ID NO: 600 - DNA, 16 bp, Artificial sequence
  Synthetic oligonucleotide
gtaatacttttggcaa

SEQ ID NO: 601 - DNA, 16 bp, Artificial sequence
  Synthetic oligonucleotide
ggtattacaaattatc

SEQ ID NO: 602 - DNA, 16 bp, Artificial sequence
  Synthetic oligonucleotide
cttaacaaccctgtca

SEQ ID NO: 603 - DNA, 16 bp, Artificial sequence
  Synthetic oligonucleotide
agtaactggtagctcc

SEQ ID NO: 604 - DNA, 16 bp, Artificial sequence
  Synthetic oligonucleotide
ctaatacccatactgc

SEQ ID NO: 605 - DNA, 16 bp, Artificial sequence
  Synthetic oligonucleotide
aactttgcagcctatc

SEQ ID NO: 606 - DNA, 16 bp, Artificial sequence
  Synthetic oligonucleotide
agaactaaggcaaatc

SEQ ID NO: 607 - DNA, 16 bp, Artificial sequence
  Synthetic oligonucleotide
gtcttggccaacgaca

SEQ ID NO: 608 - DNA, 16 bp, Artificial sequence
  Synthetic oligonucleotide
caggatgcaatccccc

SEQ ID NO: 609 - DNA, 16 bp, Artificial sequence
  Synthetic oligonucleotide
gccaatacatagcaga

SEQ ID NO: 610 - DNA, 16 bp, Artificial sequence
  Synthetic oligonucleotide
gtccatgaaatccagg

SEQ ID NO: 611 - DNA, 16 bp, Artificial sequence
  Synthetic oligonucleotide
tctcttagggcacctc

SEQ ID NO: 612 - DNA, 16 bp, Artificial sequence
  Synthetic oligonucleotide
tgaatataccccacag

SEQ ID NO: 613 - DNA, 16 bp, Artificial sequence
  Synthetic oligonucleotide
agctctaggagtcccc

SEQ ID NO: 614 - DNA, 16 bp, Artificial sequence
  Synthetic oligonucleotide
ccagattgagtctcct

SEQ ID NO: 615 - DNA, 16 bp, Artificial sequence
  Synthetic oligonucleotide
aatcaagtgccctcca

SEQ ID NO: 616 - DNA, 16 bp, Artificial sequence
  Synthetic oligonucleotide
tgtagctgtgtggtgg

SEQ ID NO: 617 - DNA, 16 bp, Artificial sequence
  Synthetic oligonucleotide
taccatgatcaggtca

SEQ ID NO: 618 - DNA, 16 bp, Artificial sequence
  Synthetic oligonucleotide
gtaaagatgtgagtga

SEQ ID NO: 619 - DNA, 16 bp, Artificial sequence
  Synthetic oligonucleotide
gtttacaaaagctgcc

SEQ ID NO: 620 - DNA, 16 bp, Artificial sequence
  Synthetic oligonucleotide
tgaactccggctcagt

SEQ ID NO: 621 - DNA, 16 bp, Artificial sequence
  Synthetic oligonucleotide
gggcattcccagcgcg

SEQ ID NO: 622 - DNA, 16 bp, Artificial sequence
  Synthetic oligonucleotide
cgctctctaccctgcc

SEQ ID NO: 623 - DNA, 16 bp, Artificial sequence
  Synthetic oligonucleotide
gatcgggaatcggctc

SEQ ID NO: 624 - DNA, 16 bp, Artificial sequence
  Synthetic oligonucleotide
cccgcgaaggacaagc

SEQ ID NO: 625 - DNA, 16 bp, Artificial sequence
  Synthetic oligonucleotide
gtcgcggaggaggtgc

SEQ ID NO: 626 - DNA, 16 bp, Artificial sequence
  Synthetic oligonucleotide
gtctgctccagcggga

SEQ ID NO: 627 - DNA, 16 bp, Artificial sequence
  Synthetic oligonucleotide
actcctggccttccgc

SEQ ID NO: 628 - DNA, 16 bp, Artificial sequence
  Synthetic oligonucleotide
cttgcttaagttgaag

SEQ ID NO: 629 - DNA, 16 bp, Artificial sequence
  Synthetic oligonucleotide
ttgcagagaccctgtc

SEQ ID NO: 630 - DNA, 16 bp, Artificial sequence
  Synthetic oligonucleotide
tgccggagatgagctg

SEQ ID NO: 631 - DNA, 16 bp, Artificial sequence
  Synthetic oligonucleotide
tggtaagagagatgcc

SEQ ID NO: 632 - DNA, 16 bp, Artificial sequence
  Synthetic oligonucleotide
ctttggaccgaaagtc

SEQ ID NO: 633 - DNA, 16 bp, Artificial sequence
  Synthetic oligonucleotide
gatggttgttttggca

SEQ ID NO: 634 - DNA, 16 bp, Artificial sequence
  Synthetic oligonucleotide
acatgaagaaagttcg

SEQ ID NO: 635 - DNA, 16 bp, Artificial sequence
  Synthetic oligonucleotide
gctctcgagagaaggt

SEQ ID NO: 636 - DNA, 16 bp, Artificial sequence
  Synthetic oligonucleotide
atatcctcgaaggcat

SEQ ID NO: 637 - DNA, 16 bp, Artificial sequence
  Synthetic oligonucleotide
cccttctgaggatgac

SEQ ID NO: 638 - DNA, 16 bp, Artificial sequence
  Synthetic oligonucleotide
gtttgcccagctgggc

SEQ ID NO: 639 - DNA, 16 bp, Artificial sequence
  Synthetic oligonucleotide
gtctagcagctcatct

SEQ ID NO: 640 - DNA, 16 bp, Artificial sequence
  Synthetic oligonucleotide
ctgtagcgagcctggg

SEQ ID NO: 641 - DNA, 16 bp, Artificial sequence
  Synthetic oligonucleotide
ggtagcaagttgcaaa

SEQ ID NO: 642 - DNA, 16 bp, Artificial sequence
  Synthetic oligonucleotide
gtacagggcagcatta

SEQ ID NO: 643 - DNA, 16 bp, Artificial sequence
  Synthetic oligonucleotide
gcaaccacaggacatc

SEQ ID NO: 644 - DNA, 16 bp, Artificial sequence
  Synthetic oligonucleotide
atcagcactcgagtga

SEQ ID NO: 645 - DNA, 16 bp, Artificial sequence
  Synthetic oligonucleotide
gggaggcctgttggct

SEQ ID NO: 646 - DNA, 16 bp, Artificial sequence
  Synthetic oligonucleotide
ctgaggatggaccgcg

SEQ ID NO: 647 - DNA, 16 bp, Artificial sequence
  Synthetic oligonucleotide
caccagcaggtacttt

SEQ ID NO: 648 - DNA, 16 bp, Artificial sequence
  Synthetic oligonucleotide
caagtgactcacagac

SEQ ID NO: 649 - DNA, 16 bp, Artificial sequence
  Synthetic oligonucleotide
tgaaagaatctgctag

SEQ ID NO: 650 - DNA, 16 bp, Artificial sequence
  Synthetic oligonucleotide
ctacacagcaatgcgg

SEQ ID NO: 651 - DNA, 16 bp, Artificial sequence
  Synthetic oligonucleotide
caggcgaaaggttgct

SEQ ID NO: 652 - DNA, 16 bp, Artificial sequence
  Synthetic oligonucleotide
gttaagtgctggaccg

SEQ ID NO: 653 - DNA, 16 bp, Artificial sequence
  Synthetic oligonucleotide
gaattaacgcatgctg

SEQ ID NO: 654 - DNA, 16 bp, Artificial sequence
  Synthetic oligonucleotide
gggaccctctgcactg

SEQ ID NO: 655 - DNA, 16 bp, Artificial sequence
  Synthetic oligonucleotide
atagggccacgaaaca

SEQ ID NO: 656 - DNA, 16 bp, Artificial sequence
  Synthetic oligonucleotide
gaacctcatgctggaa

SEQ ID NO: 657 - DNA, 16 bp, Artificial sequence
  Synthetic oligonucleotide
cccatcacaaggcccc

SEQ ID NO: 658 - DNA, 16 bp, Artificial sequence
  Synthetic oligonucleotide
tcacacatgggccagc

SEQ ID NO: 659 - DNA, 16 bp, Artificial sequence
  Synthetic oligonucleotide
ctgacaggcagtgtcg

SEQ ID NO: 660 - DNA, 16 bp, Artificial sequence
  Synthetic oligonucleotide
agtcaaggttatcatc

SEQ ID NO: 661 - DNA, 16 bp, Artificial sequence
  Synthetic oligonucleotide
atcatcttgttacccc

SEQ ID NO: 662 - DNA, 16 bp, Artificial sequence
  Synthetic oligonucleotide
attctactaacatctc

SEQ ID NO: 663 - DNA, 16 bp, Artificial sequence
  Synthetic oligonucleotide
gaatgccctgcacact

SEQ ID NO: 664 - DNA, 16 bp, Artificial sequence
  Synthetic oligonucleotide
gctccagtggagagga

SEQ ID NO: 665 - DNA, 16 bp, Artificial sequence
  Synthetic oligonucleotide
ataagccaggttcaag

SEQ ID NO: 666 - DNA, 16 bp, Artificial sequence
  Synthetic oligonucleotide
gtgagaaacaaaccct

SEQ ID NO: 667 - DNA, 16 bp, Artificial sequence
  Synthetic oligonucleotide
tctataccagagtgag

SEQ ID NO: 668 - DNA, 16 bp, Artificial sequence
  Synthetic oligonucleotide
aggaatgagtctccca

SEQ ID NO: 669 - DNA, 16 bp, Artificial sequence
  Synthetic oligonucleotide
ggtaatacttttggca

SEQ ID NO: 670 - DNA, 16 bp, Artificial sequence
  Synthetic oligonucleotide
cgcttatgaaagcatc

SEQ ID NO: 671 - DNA, 16 bp, Artificial sequence
  Synthetic oligonucleotide
cccttaacaaccctgt

SEQ ID NO: 672 - DNA, 16 bp, Artificial sequence
  Synthetic oligonucleotide
tttgattgtgcagaca

SEQ ID NO: 673 - DNA, 16 bp, Artificial sequence
  Synthetic oligonucleotide
tcctaatacccatact

SEQ ID NO: 674 - DNA, 16 bp, Artificial sequence
  Synthetic oligonucleotide
acaaactttgcagcct

SEQ ID NO: 675 - DNA, 16 bp, Artificial sequence
  Synthetic oligonucleotide
gttagaactaaggcaa

SEQ ID NO: 676 - DNA, 16 bp, Artificial sequence
  Synthetic oligonucleotide
gagcagataaatacac

SEQ ID NO: 677 - DNA, 16 bp, Artificial sequence
  Synthetic oligonucleotide
tggtatctcgcttcct

SEQ ID NO: 678 - DNA, 16 bp, Artificial sequence
  Synthetic oligonucleotide
taaagccacgcagcca

SEQ ID NO: 679 - DNA, 16 bp, Artificial sequence
  Synthetic oligonucleotide
ccagatgcaggacccc

SEQ ID NO: 680 - DNA, 16 bp, Artificial sequence
  Synthetic oligonucleotide
aaactaatgcacctgg

SEQ ID NO: 681 - DNA, 16 bp, Artificial sequence
  Synthetic oligonucleotide
ctgaatataccccaca

SEQ ID NO: 682 - DNA, 16 bp, Artificial sequence
  Synthetic oligonucleotide
agctgctatgtgaggc

SEQ ID NO: 683 - DNA, 16 bp, Artificial sequence
  Synthetic oligonucleotide
tcagtaaccagattga

SEQ ID NO: 684 - DNA, 16 bp, Artificial sequence
  Synthetic oligonucleotide
tttaatcaagtgccct

SEQ ID NO: 685 - DNA, 16 bp, Artificial sequence
  Synthetic oligonucleotide
caggatgtagctgtgt

SEQ ID NO: 686 - DNA, 16 bp, Artificial sequence
  Synthetic oligonucleotide
taagatcccatcttac

SEQ ID NO: 687 - DNA, 16 bp, Artificial sequence
  Synthetic oligonucleotide
aaagtaaacacccacc

SEQ ID NO: 688 - DNA, 16 bp, Artificial sequence
  Synthetic oligonucleotide
gcttacaacactaccc

SEQ ID NO: 689 - DNA, 16 bp, Artificial sequence
  Synthetic oligonucleotide
gtaatgggagccaggc

SEQ ID NO: 690 - DNA, 16 bp, Artificial sequence
  Synthetic oligonucleotide
agggcattcccagcgc

SEQ ID NO: 691 - DNA, 16 bp, Artificial sequence
  Synthetic oligonucleotide
gcgctctctaccctgc

SEQ ID NO: 692 - DNA, 16 bp, Artificial sequence
  Synthetic oligonucleotide
ggatcgggaatcggct

SEQ ID NO: 693 - DNA, 16 bp, Artificial sequence
  Synthetic oligonucleotide
gcccgcgaaggacaag

SEQ ID NO: 694 - DNA, 16 bp, Artificial sequence
  Synthetic oligonucleotide
cgtcgcggaggaggtg

SEQ ID NO: 695 - DNA, 16 bp, Artificial sequence
  Synthetic oligonucleotide
aggacctgcagagtct

SEQ ID NO: 696 - DNA, 16 bp, Artificial sequence
  Synthetic oligonucleotide
cgactcctggccttcc

SEQ ID NO: 697 - DNA, 16 bp, Artificial sequence
  Synthetic oligonucleotide
acttgcttaagttgaa

SEQ ID NO: 698 - DNA, 16 bp, Artificial sequence
  Synthetic oligonucleotide
gggaggcatttgcaga

SEQ ID NO: 699 - DNA, 16 bp, Artificial sequence
  Synthetic oligonucleotide
ttgccggagatgagct

SEQ ID NO: 700 - DNA, 16 bp, Artificial sequence
  Synthetic oligonucleotide
tctggtaagagagatg

SEQ ID NO: 701 - DNA, 16 bp, Artificial sequence
  Synthetic oligonucleotide
tctttggaccgaaagt

SEQ ID NO: 702 - DNA, 16 bp, Artificial sequence
  Synthetic oligonucleotide
tgatggttgttttggc

SEQ ID NO: 703 - DNA, 16 bp, Artificial sequence
  Synthetic oligonucleotide
gtccacatgaagaaag

SEQ ID NO: 704 - DNA, 16 bp, Artificial sequence
  Synthetic oligonucleotide
agctctcgagagaagg

SEQ ID NO: 705 - DNA, 16 bp, Artificial sequence
  Synthetic oligonucleotide
aatatcctcgaaggca

SEQ ID NO: 706 - DNA, 16 bp, Artificial sequence
  Synthetic oligonucleotide
gatccatcccttctga

SEQ ID NO: 707 - DNA, 16 bp, Artificial sequence
  Synthetic oligonucleotide
tgtttgcccagctggg

SEQ ID NO: 708 - DNA, 16 bp, Artificial sequence
  Synthetic oligonucleotide
gacgcaggtggtctag

SEQ ID NO: 709 - DNA, 16 bp, Artificial sequence
  Synthetic oligonucleotide
gctgtagcgagcctgg

SEQ ID NO: 710 - DNA, 16 bp, Artificial sequence
  Synthetic oligonucleotide
tgggtagcaagttgca

SEQ ID NO: 711 - DNA, 16 bp, Artificial sequence
  Synthetic oligonucleotide
ggtacagggcagcatt

SEQ ID NO: 712 - DNA, 16 bp, Artificial sequence
  Synthetic oligonucleotide
tgcaaccacaggacat

SEQ ID NO: 713 - DNA, 16 bp, Artificial sequence
  Synthetic oligonucleotide
catcagcactcgagtg

SEQ ID NO: 714 - DNA, 16 bp, Artificial sequence
  Synthetic oligonucleotide
ctcaggtgtgcatggg

SEQ ID NO: 715 - DNA, 16 bp, Artificial sequence
  Synthetic oligonucleotide
cctgaggatggaccgc

SEQ ID NO: 716 - DNA, 16 bp, Artificial sequence
  Synthetic oligonucleotide
agcaccagcaggtact

SEQ ID NO: 717 - DNA, 16 bp, Artificial sequence
  Synthetic oligonucleotide
tcaagtgactcacaga

SEQ ID NO: 718 - DNA, 16 bp, Artificial sequence
  Synthetic oligonucleotide
cacctctgaaagaatc

SEQ ID NO: 719 - DNA, 16 bp, Artificial sequence
  Synthetic oligonucleotide
actacacagcaatgcg

SEQ ID NO: 720 - DNA, 16 bp, Artificial sequence
  Synthetic oligonucleotide
acaggcgaaaggttgc

SEQ ID NO: 721 - DNA, 16 bp, Artificial sequence
  Synthetic oligonucleotide
agttaagtgctggacc

SEQ ID NO: 722 - DNA, 16 bp, Artificial sequence
  Synthetic oligonucleotide
gctgaattaacgcatg

SEQ ID NO: 723 - DNA, 16 bp, Artificial sequence
  Synthetic oligonucleotide
agggaccctctgcact

SEQ ID NO: 724 - DNA, 16 bp, Artificial sequence
  Synthetic oligonucleotide
aatagggccacgaaac

SEQ ID NO: 725 - DNA, 16 bp, Artificial sequence
  Synthetic oligonucleotide
agaacctcatgctgga

SEQ ID NO: 726 - DNA, 16 bp, Artificial sequence
  Synthetic oligonucleotide
ccccatcacaaggccc

SEQ ID NO: 727 - DNA, 16 bp, Artificial sequence
  Synthetic oligonucleotide
atcacacatgggccag

SEQ ID NO: 728 - DNA, 16 bp, Artificial sequence
  Synthetic oligonucleotide
cctgacaggcagtgtc

SEQ ID NO: 729 - DNA, 16 bp, Artificial sequence
  Synthetic oligonucleotide
tagtcaaggttatcat

SEQ ID NO: 730 - DNA, 16 bp, Artificial sequence
  Synthetic oligonucleotide
ttatcatcttgttacc

SEQ ID NO: 731 - DNA, 16 bp, Artificial sequence
  Synthetic oligonucleotide
cttattctactaacat

SEQ ID NO: 732 - DNA, 16 bp, Artificial sequence
  Synthetic oligonucleotide
tgaatgccctgcacac

SEQ ID NO: 733 - DNA, 16 bp, Artificial sequence
  Synthetic oligonucleotide
tgctccagtggagagg

SEQ ID NO: 734 - DNA, 16 bp, Artificial sequence
  Synthetic oligonucleotide
gtccctgcagaaaata

SEQ ID NO: 735 - DNA, 16 bp, Artificial sequence
  Synthetic oligonucleotide
agcataccacacccca

SEQ ID NO: 736 - DNA, 16 bp, Artificial sequence
  Synthetic oligonucleotide
ggacatgctcagcagc

SEQ ID NO: 737 - DNA, 16 bp, Artificial sequence
  Synthetic oligonucleotide
tgctgtaggcctcagc

SEQ ID NO: 738 - DNA, 16 bp, Artificial sequence
  Synthetic oligonucleotide
tggtaatacttttggc

SEQ ID NO: 739 - DNA, 16 bp, Artificial sequence
  Synthetic oligonucleotide
gtaaatggagtccttc

SEQ ID NO: 740 - DNA, 16 bp, Artificial sequence
  Synthetic oligonucleotide
cataatccccttaaca

SEQ ID NO: 741 - DNA, 16 bp, Artificial sequence
  Synthetic oligonucleotide
ttaaccatcaaggaca

SEQ ID NO: 742 - DNA, 16 bp, Artificial sequence
  Synthetic oligonucleotide
tcttagtggctgggta

SEQ ID NO: 743 - DNA, 16 bp, Artificial sequence
  Synthetic oligonucleotide
cctaacaaactttgca

SEQ ID NO: 744 - DNA, 16 bp, Artificial sequence
  Synthetic oligonucleotide
actaagtgttagaact

SEQ ID NO: 745 - DNA, 16 bp, Artificial sequence
  Synthetic oligonucleotide
ctgcagtatccctagc

SEQ ID NO: 746 - DNA, 16 bp, Artificial sequence
  Synthetic oligonucleotide
tcccatcggtcatttc

SEQ ID NO: 747 - DNA, 16 bp, Artificial sequence
  Synthetic oligonucleotide
gaaaccactatcatca

SEQ ID NO: 748 - DNA, 16 bp, Artificial sequence
  Synthetic oligonucleotide
gtaataggccaagtcc

SEQ ID NO: 749 - DNA, 16 bp, Artificial sequence
  Synthetic oligonucleotide
caaactaatgcacctg

SEQ ID NO: 750 - DNA, 16 bp, Artificial sequence
  Synthetic oligonucleotide
ccaatatcatagctga

SEQ ID NO: 751 - DNA, 16 bp, Artificial sequence
  Synthetic oligonucleotide
cacaagagactggacc

SEQ ID NO: 752 - DNA, 16 bp, Artificial sequence
  Synthetic oligonucleotide
tactatgggatgagta

SEQ ID NO: 753 - DNA, 16 bp, Artificial sequence
  Synthetic oligonucleotide
ttttaatcaagtgccc

SEQ ID NO: 754 - DNA, 16 bp, Artificial sequence
  Synthetic oligonucleotide
ggcaggatgtagctgt

SEQ ID NO: 755 - DNA, 16 bp, Artificial sequence
  Synthetic oligonucleotide
agtcaaacatcttcct

SEQ ID NO: 756 - DNA, 16 bp, Artificial sequence
  Synthetic oligonucleotide
cagactaacttactaa

SEQ ID NO: 757 - DNA, 16 bp, Artificial sequence
  Synthetic oligonucleotide
agcttacaacactacc

SEQ ID NO: 758 - DNA, 16 bp, Artificial sequence
  Synthetic oligonucleotide
atgctacgggctctca

SEQ ID NO: 759 - DNA, 16 bp, Artificial sequence
  Synthetic oligonucleotide
cagggcattcccagcg

SEQ ID NO: 760 - DNA, 16 bp, Artificial sequence
  Synthetic oligonucleotide
cagctccgcccggcgc

SEQ ID NO: 761 - DNA, 16 bp, Artificial sequence
  Synthetic oligonucleotide
ttaggatctgggtcgg

SEQ ID NO: 762 - DNA, 16 bp, Artificial sequence
  Synthetic oligonucleotide
agcccgcgaaggacaa

SEQ ID NO: 763 - DNA, 16 bp, Artificial sequence
  Synthetic oligonucleotide
gcgccgaacaacatgc

SEQ ID NO: 764 - DNA, 16 bp, Artificial sequence
  Synthetic oligonucleotide
agaggacctgcagagt

SEQ ID NO: 765 - DNA, 16 bp, Artificial sequence
  Synthetic oligonucleotide
ccgactcctggccttc

SEQ ID NO: 766 - DNA, 16 bp, Artificial sequence
  Synthetic oligonucleotide
aacttgcttaagttga

SEQ ID NO: 767 - DNA, 16 bp, Artificial sequence
  Synthetic oligonucleotide
cgggaggcatttgcag

SEQ ID NO: 768 - DNA, 16 bp, Artificial sequence
  Synthetic oligonucleotide
tttgccggagatgagc

SEQ ID NO: 769 - DNA, 16 bp, Artificial sequence
  Synthetic oligonucleotide
ctctggtaagagagat

SEQ ID NO: 770 - DNA, 16 bp, Artificial sequence
  Synthetic oligonucleotide
gtctttggaccgaaag

SEQ ID NO: 771 - DNA, 16 bp, Artificial sequence
  Synthetic oligonucleotide
gagggataaggccact

SEQ ID NO: 772 - DNA, 16 bp, Artificial sequence
  Synthetic oligonucleotide
gtgatggttgttttgg

SEQ ID NO: 773 - DNA, 16 bp, Artificial sequence
  Synthetic oligonucleotide
ggtgatgtccacatga

SEQ ID NO: 774 - DNA, 16 bp, Artificial sequence
  Synthetic oligonucleotide
aagctctcgagagaag

SEQ ID NO: 775 - DNA, 16 bp, Artificial sequence
  Synthetic oligonucleotide
caaatatcctcgaagg

SEQ ID NO: 776 - DNA, 16 bp, Artificial sequence
  Synthetic oligonucleotide
ggatccatcccttctg

SEQ ID NO: 777 - DNA, 16 bp, Artificial sequence
  Synthetic oligonucleotide
ctcatgtttgcccagc

SEQ ID NO: 778 - DNA, 16 bp, Artificial sequence
  Synthetic oligonucleotide
agacgcaggtggtcta

SEQ ID NO: 779 - DNA, 16 bp, Artificial sequence
  Synthetic oligonucleotide
tgctgtagcgagcctg

SEQ ID NO: 780 - DNA, 16 bp, Artificial sequence
  Synthetic oligonucleotide
atgggtagcaagttgc

SEQ ID NO: 781 - DNA, 16 bp, Artificial sequence
  Synthetic oligonucleotide
ttccacaggcagggta

SEQ ID NO: 782 - DNA, 16 bp, Artificial sequence
  Synthetic oligonucleotide
actgcaaccacaggac

SEQ ID NO: 783 - DNA, 16 bp, Artificial sequence
  Synthetic oligonucleotide
acatcagcactcgagt

SEQ ID NO: 784 - DNA, 16 bp, Artificial sequence
  Synthetic oligonucleotide
ctgctcaggtgtgcat

SEQ ID NO: 785 - DNA, 16 bp, Artificial sequence
  Synthetic oligonucleotide
acctgaggatggaccg

SEQ ID NO: 786 - DNA, 16 bp, Artificial sequence
  Synthetic oligonucleotide
cagcaccagcaggtac

SEQ ID NO: 787 - DNA, 16 bp, Artificial sequence
  Synthetic oligonucleotide
ctcctcaagtgactca

SEQ ID NO: 788 - DNA, 16 bp, Artificial sequence
  Synthetic oligonucleotide
tagcacctctgaaaga

SEQ ID NO: 789 - DNA, 16 bp, Artificial sequence
  Synthetic oligonucleotide
cactacacagcaatgc

SEQ ID NO: 790 - DNA, 16 bp, Artificial sequence
  Synthetic oligonucleotide
cacaggcgaaaggttg

SEQ ID NO: 791 - DNA, 16 bp, Artificial sequence
  Synthetic oligonucleotide
agagttaagtgctgga

SEQ ID NO: 792 - DNA, 16 bp, Artificial sequence
  Synthetic oligonucleotide
agctgaattaacgcat

SEQ ID NO: 793 - DNA, 16 bp, Artificial sequence
  Synthetic oligonucleotide
aagggaccctctgcac

SEQ ID NO: 794 - DNA, 16 bp, Artificial sequence
  Synthetic oligonucleotide
taatagggccacgaaa

SEQ ID NO: 795 - DNA, 16 bp, Artificial sequence
  Synthetic oligonucleotide
aagaacctcatgctgg

SEQ ID NO: 796 - DNA, 16 bp, Artificial sequence
  Synthetic oligonucleotide
cccccatcacaaggcc

SEQ ID NO: 797 - DNA, 16 bp, Artificial sequence
  Synthetic oligonucleotide
gatcacacatgggcca

SEQ ID NO: 798 - DNA, 16 bp, Artificial sequence
  Synthetic oligonucleotide
acctgacaggcagtgt

SEQ ID NO: 799 - DNA, 16 bp, Artificial sequence
  Synthetic oligonucleotide
gtagtcaaggttatca

SEQ ID NO: 800 - DNA, 16 bp, Artificial sequence
  Synthetic oligonucleotide
taagtagattatcatc

SEQ ID NO: 801 - DNA, 16 bp, Artificial sequence
  Synthetic oligonucleotide
aggcttattctactaa

SEQ ID NO: 802 - DNA, 16 bp, Artificial sequence
  Synthetic oligonucleotide
gtgaatgccctgcaca

SEQ ID NO: 803 - DNA, 16 bp, Artificial sequence
  Synthetic oligonucleotide
gtgctccagtggagag

SEQ ID NO: 804 - DNA, 16 bp, Artificial sequence
  Synthetic oligonucleotide
ggtccctgcagaaaat

SEQ ID NO: 805 - DNA, 16 bp, Artificial sequence
  Synthetic oligonucleotide
aagcataccacacccc

SEQ ID NO: 806 - DNA, 16 bp, Artificial sequence
  Synthetic oligonucleotide
aatcttgggatgcaca

SEQ ID NO: 807 - DNA, 16 bp, Artificial sequence
  Synthetic oligonucleotide
catcatggcttccagt

SEQ ID NO: 808 - DNA, 16 bp, Artificial sequence
  Synthetic oligonucleotide
tgggatggtaatactt

SEQ ID NO: 809 - DNA, 16 bp, Artificial sequence
  Synthetic oligonucleotide
aagtaaatggagtcct

SEQ ID NO: 810 - DNA, 16 bp, Artificial sequence
  Synthetic oligonucleotide
ttgcataatcccctta

SEQ ID NO: 811 - DNA, 16 bp, Artificial sequence
  Synthetic oligonucleotide
ttaactagatcactga

SEQ ID NO: 812 - DNA, 16 bp, Artificial sequence
  Synthetic oligonucleotide
tcctaatgcgagtccc

SEQ ID NO: 813 - DNA, 16 bp, Artificial sequence
  Synthetic oligonucleotide
tgctgctgggtgcact

SEQ ID NO: 814 - DNA, 16 bp, Artificial sequence
  Synthetic oligonucleotide
ggtgatgacacagcat

SEQ ID NO: 815 - DNA, 16 bp, Artificial sequence
  Synthetic oligonucleotide
gccatgtacaactttt

SEQ ID NO: 816 - DNA, 16 bp, Artificial sequence
  Synthetic oligonucleotide
tacaatttggacagag

SEQ ID NO: 817 - DNA, 16 bp, Artificial sequence
  Synthetic oligonucleotide
acctataggagtgccc

SEQ ID NO: 818 - DNA, 16 bp, Artificial sequence
  Synthetic oligonucleotide
ttatttccgttaacca

SEQ ID NO: 819 - DNA, 16 bp, Artificial sequence
  Synthetic oligonucleotide
agaatcatcttagtgg

SEQ ID NO: 820 - DNA, 16 bp, Artificial sequence
  Synthetic oligonucleotide
cggaataagcctccac

SEQ ID NO: 821 - DNA, 16 bp, Artificial sequence
  Synthetic oligonucleotide
ggcactaagtgttaga

SEQ ID NO: 822 - DNA, 16 bp, Artificial sequence
  Synthetic oligonucleotide
tctcacaaggctggca

SEQ ID NO: 823 - DNA, 16 bp, Artificial sequence
  Synthetic oligonucleotide
gccataccggctccct

SEQ ID NO: 824 - DNA, 16 bp, Artificial sequence
  Synthetic oligonucleotide
ggccttacagaaacca

SEQ ID NO: 825 - DNA, 16 bp, Artificial sequence
  Synthetic oligonucleotide
agtaataggccaagtc

SEQ ID NO: 826 - DNA, 16 bp, Artificial sequence
  Synthetic oligonucleotide
acaaactaatgcacct

SEQ ID NO: 827 - DNA, 16 bp, Artificial sequence
  Synthetic oligonucleotide
tccaatatcatagctg

SEQ ID NO: 828 - DNA, 16 bp, Artificial sequence
  Synthetic oligonucleotide
ctactaggcatctcta

SEQ ID NO: 829 - DNA, 16 bp, Artificial sequence
  Synthetic oligonucleotide
cttactatgggatgag

SEQ ID NO: 830 - DNA, 16 bp, Artificial sequence
  Synthetic oligonucleotide
taatattcagaccagg

SEQ ID NO: 831 - DNA, 16 bp, Artificial sequence
  Synthetic oligonucleotide
ccatgcatggcacagt

SEQ ID NO: 832 - DNA, 16 bp, Artificial sequence
  Synthetic oligonucleotide
agacaggaatccaacc

SEQ ID NO: 833 - DNA, 16 bp, Artificial sequence
  Synthetic oligonucleotide
ggacatgacagactaa

SEQ ID NO: 834 - DNA, 16 bp, Artificial sequence
  Synthetic oligonucleotide
gcagacacaacagctt

SEQ ID NO: 835 - DNA, 16 bp, Artificial sequence
  Synthetic oligonucleotide
ccatgctacgggctct

SEQ ID NO: 836 - DNA, 16 bp, Artificial sequence
  Synthetic oligonucleotide
ggccagggcattccca

SEQ ID NO: 837 - DNA, 16 bp, Artificial sequence
  Synthetic oligonucleotide
gcagctccgcccggcg

SEQ ID NO: 838 - DNA, 16 bp, Artificial sequence
  Synthetic oligonucleotide
ggttaggatctgggtc

SEQ ID NO: 839 - DNA, 16 bp, Artificial sequence
  Synthetic oligonucleotide
ggtagaagcccaggaa

SEQ ID NO: 840 - DNA, 16 bp, Artificial sequence
  Synthetic oligonucleotide
cgacgcagtgcaacgc

SEQ ID NO: 841 - DNA, 16 bp, Artificial sequence
  Synthetic oligonucleotide
tgagaggacctgcaga

SEQ ID NO: 842 - DNA, 16 bp, Artificial sequence
  Synthetic oligonucleotide
atgttccgactcctgg

SEQ ID NO: 843 - DNA, 16 bp, Artificial sequence
  Synthetic oligonucleotide
gaacttgcttaagttg

SEQ ID NO: 844 - DNA, 16 bp, Artificial sequence
  Synthetic oligonucleotide
ccgggaggcatttgca

SEQ ID NO: 845 - DNA, 16 bp, Artificial sequence
  Synthetic oligonucleotide
ttttgccggagatgag

SEQ ID NO: 846 - DNA, 16 bp, Artificial sequence
  Synthetic oligonucleotide
actctggtaagagaga

SEQ ID NO: 847 - DNA, 16 bp, Artificial sequence
  Synthetic oligonucleotide
cgtctttggaccgaaa

SEQ ID NO: 848 - DNA, 16 bp, Artificial sequence
  Synthetic oligonucleotide
cggtgatggttgtttt

SEQ ID NO: 849 - DNA, 16 bp, Artificial sequence
  Synthetic oligonucleotide
tggtgatgtccacatg

SEQ ID NO: 850 - DNA, 16 bp, Artificial sequence
  Synthetic oligonucleotide
aaagctctcgagagaa

SEQ ID NO: 851 - DNA, 16 bp, Artificial sequence
  Synthetic oligonucleotide
atccaaatatcctcga

SEQ ID NO: 852 - DNA, 16 bp, Artificial sequence
  Synthetic oligonucleotide
aggatccatcccttct

SEQ ID NO: 853 - DNA, 16 bp, Artificial sequence
  Synthetic oligonucleotide
gactcatgtttgccca

SEQ ID NO: 854 - DNA, 16 bp, Artificial sequence
  Synthetic oligonucleotide
gagacgcaggtggtct

SEQ ID NO: 855 - DNA, 16 bp, Artificial sequence
  Synthetic oligonucleotide
gtgctgtagcgagcct

SEQ ID NO: 856 - DNA, 16 bp, Artificial sequence
  Synthetic oligonucleotide
aatgggtagcaagttg

SEQ ID NO: 857 - DNA, 16 bp, Artificial sequence
  Synthetic oligonucleotide
attccacaggcagggt

SEQ ID NO: 858 - DNA, 16 bp, Artificial sequence
  Synthetic oligonucleotide
gtcacccactgcaacc

SEQ ID NO: 859 - DNA, 16 bp, Artificial sequence
  Synthetic oligonucleotide
cacatcagcactcgag

SEQ ID NO: 860 - DNA, 16 bp, Artificial sequence
  Synthetic oligonucleotide
tcctgctcaggtgtgc

SEQ ID NO: 861 - DNA, 16 bp, Artificial sequence
  Synthetic oligonucleotide
gacctgaggatggacc

SEQ ID NO: 862 - DNA, 16 bp, Artificial sequence
  Synthetic oligonucleotide
tcagcaccagcaggta

SEQ ID NO: 863 - DNA, 16 bp, Artificial sequence
  Synthetic oligonucleotide
cgcctcctcaagtgac

SEQ ID NO: 864 - DNA, 16 bp, Artificial sequence
  Synthetic oligonucleotide
actttagcacctctga

SEQ ID NO: 865 - DNA, 16 bp, Artificial sequence
  Synthetic oligonucleotide
gtcactacacagcaat

SEQ ID NO: 866 - DNA, 16 bp, Artificial sequence
  Synthetic oligonucleotide
gcacaggcgaaaggtt

SEQ ID NO: 867 - DNA, 16 bp, Artificial sequence
  Synthetic oligonucleotide
tagagttaagtgctgg

SEQ ID NO: 868 - DNA, 16 bp, Artificial sequence
  Synthetic oligonucleotide
cagctgaattaacgca

SEQ ID NO: 869 - DNA, 16 bp, Artificial sequence
  Synthetic oligonucleotide
taagggaccctctgca

SEQ ID NO: 870 - DNA, 16 bp, Artificial sequence
  Synthetic oligonucleotide
ttaatagggccacgaa

SEQ ID NO: 871 - DNA, 16 bp, Artificial sequence
  Synthetic oligonucleotide
taagaacctcatgctg

SEQ ID NO: 872 - DNA, 16 bp, Artificial sequence
  Synthetic oligonucleotide
ccccccatcacaaggc

SEQ ID NO: 873 - DNA, 16 bp, Artificial sequence
  Synthetic oligonucleotide
agatcacacatgggcc

SEQ ID NO: 874 - DNA, 16 bp, Artificial sequence
  Synthetic oligonucleotide
accacctgacaggcag

SEQ ID NO: 875 - DNA, 16 bp, Artificial sequence
  Synthetic oligonucleotide
agtagtcaaggttatc

SEQ ID NO: 876 - DNA, 16 bp, Artificial sequence
  Synthetic oligonucleotide
tgaaaaaggtgttcta

SEQ ID NO: 877 - DNA, 16 bp, Artificial sequence
  Synthetic oligonucleotide
aaggcttattctacta

SEQ ID NO: 878 - DNA, 16 bp, Artificial sequence
  Synthetic oligonucleotide
aggtgaatgccctgca

SEQ ID NO: 879 - DNA, 16 bp, Artificial sequence
  Synthetic oligonucleotide
tgtgctccagtggaga

SEQ ID NO: 880 - DNA, 16 bp, Artificial sequence
  Synthetic oligonucleotide
tggtccctgcagaaaa

SEQ ID NO: 881 - DNA, 16 bp, Artificial sequence
  Synthetic oligonucleotide
tgcctactggctcaca

SEQ ID NO: 882 - DNA, 16 bp, Artificial sequence
  Synthetic oligonucleotide
aaatcttgggatgcac

SEQ ID NO: 883 - DNA, 16 bp, Artificial sequence
  Synthetic oligonucleotide
tgacatcatggcttcc

SEQ ID NO: 884 - DNA, 16 bp, Artificial sequence
  Synthetic oligonucleotide
gcttacatccacgact

SEQ ID NO: 885 - DNA, 16 bp, Artificial sequence
  Synthetic oligonucleotide
caagtaaatggagtcc

SEQ ID NO: 886 - DNA, 16 bp, Artificial sequence
  Synthetic oligonucleotide
atctattgcataatcc

SEQ ID NO: 887 - DNA, 16 bp, Artificial sequence
  Synthetic oligonucleotide
tttatttccgttaacc

SEQ ID NO: 888 - DNA, 16 bp, Artificial sequence
  Synthetic oligonucleotide
ttcttgaccgtgtttc

SEQ ID NO: 889 - DNA, 16 bp, Artificial sequence
  Synthetic oligonucleotide
ccggaataagcctcca

SEQ ID NO: 890 - DNA, 16 bp, Artificial sequence
  Synthetic oligonucleotide
tgtacaatgggacgga

SEQ ID NO: 891 - DNA, 16 bp, Artificial sequence
  Synthetic oligonucleotide
atcgacacagcatcac

SEQ ID NO: 892 - DNA, 16 bp, Artificial sequence
  Synthetic oligonucleotide
tgccataccggctccc

SEQ ID NO: 893 - DNA, 16 bp, Artificial sequence
  Synthetic oligonucleotide
ggtttataacaactga

SEQ ID NO: 894 - DNA, 16 bp, Artificial sequence
  Synthetic oligonucleotide
gccttgaggtgggtgg

SEQ ID NO: 895 - DNA, 16 bp, Artificial sequence
  Synthetic oligonucleotide
agtcatgggatgtgca

SEQ ID NO: 896 - DNA, 16 bp, Artificial sequence
  Synthetic oligonucleotide
atgtttggaagtcgcc

SEQ ID NO: 897 - DNA, 16 bp, Artificial sequence
  Synthetic oligonucleotide
aaggatcctgcttcta

SEQ ID NO: 898 - DNA, 16 bp, Artificial sequence
  Synthetic oligonucleotide
gcttactatgggatga

SEQ ID NO: 899 - DNA, 16 bp, Artificial sequence
  Synthetic oligonucleotide
gtaatattcagaccag

SEQ ID NO: 900 - DNA, 16 bp, Artificial sequence
  Synthetic oligonucleotide
atccatgcatggcaca

SEQ ID NO: 901 - DNA, 16 bp, Artificial sequence
  Synthetic oligonucleotide
gtcagacacggagaca

SEQ ID NO: 902 - DNA, 16 bp, Artificial sequence
  Synthetic oligonucleotide
ggcttttgaaggagag

SEQ ID NO: 903 - DNA, 16 bp, Artificial sequence
  Synthetic oligonucleotide
tatttacctggaggcg

SEQ ID NO: 904 - DNA, 16 bp, Artificial sequence
  Synthetic oligonucleotide
caaatcggatctttgc

SEQ ID NO: 905 - DNA, 16 bp, Artificial sequence
  Synthetic oligonucleotide
cggccagggcattccc

SEQ ID NO: 906 - DNA, 16 bp, Artificial sequence
  Synthetic oligonucleotide
gcagcagctccgcccg

SEQ ID NO: 907 - DNA, 16 bp, Artificial sequence
  Synthetic oligonucleotide
gcgggttaggatctgg

SEQ ID NO: 908 - DNA, 16 bp, Artificial sequence
  Synthetic oligonucleotide
cgtggtagaagcccag

SEQ ID NO: 909 - DNA, 16 bp, Artificial sequence
  Synthetic oligonucleotide
ccgacgcagtgcaacg

SEQ ID NO: 910 - DNA, 16 bp, Artificial sequence
  Synthetic oligonucleotide
atctgagaggacctgc

SEQ ID NO: 911 - DNA, 16 bp, Artificial sequence
  Synthetic oligonucleotide
aatgttccgactcctg

SEQ ID NO: 912 - DNA, 16 bp, Artificial sequence
  Synthetic oligonucleotide
ggaacttgcttaagtt

SEQ ID NO: 913 - DNA, 16 bp, Artificial sequence
  Synthetic oligonucleotide
gccgggaggcatttgc

SEQ ID NO: 914 - DNA, 16 bp, Artificial sequence
  Synthetic oligonucleotide
attttgccggagatga

SEQ ID NO: 915 - DNA, 16 bp, Artificial sequence
  Synthetic oligonucleotide
cactctggtaagagag

SEQ ID NO: 916 - DNA, 16 bp, Artificial sequence
  Synthetic oligonucleotide
acggtgatggttgttt

SEQ ID NO: 917 - DNA, 16 bp, Artificial sequence
  Synthetic oligonucleotide
agcttggtgatgtcca

SEQ ID NO: 918 - DNA, 16 bp, Artificial sequence
  Synthetic oligonucleotide
aaaagctctcgagaga

SEQ ID NO: 919 - DNA, 16 bp, Artificial sequence
  Synthetic oligonucleotide
catccaaatatcctcg

SEQ ID NO: 920 - DNA, 16 bp, Artificial sequence
  Synthetic oligonucleotide
caggatccatcccttc

SEQ ID NO: 921 - DNA, 16 bp, Artificial sequence
  Synthetic oligonucleotide
agactcatgtttgccc

SEQ ID NO: 922 - DNA, 16 bp, Artificial sequence
  Synthetic oligonucleotide
ctgagacgcaggtggt

SEQ ID NO: 923 - DNA, 16 bp, Artificial sequence
  Synthetic oligonucleotide
agtgctgtagcgagcc

SEQ ID NO: 924 - DNA, 16 bp, Artificial sequence
  Synthetic oligonucleotide
taatgggtagcaagtt

SEQ ID NO: 925 - DNA, 16 bp, Artificial sequence
  Synthetic oligonucleotide
caatggcagattccac

SEQ ID NO: 926 - DNA, 16 bp, Artificial sequence
  Synthetic oligonucleotide
ggtcacccactgcaac

SEQ ID NO: 927 - DNA, 16 bp, Artificial sequence
  Synthetic oligonucleotide
acacatcagcactcga

SEQ ID NO: 928 - DNA, 16 bp, Artificial sequence
  Synthetic oligonucleotide
gtcctgctcaggtgtg

SEQ ID NO: 929 - DNA, 16 bp, Artificial sequence
  Synthetic oligonucleotide
ggctggacctgaggat

SEQ ID NO: 930 - DNA, 16 bp, Artificial sequence
  Synthetic oligonucleotide
gtggagagcccctcag

SEQ ID NO: 931 - DNA, 16 bp, Artificial sequence
  Synthetic oligonucleotide
tcgcctcctcaagtga

SEQ ID NO: 932 - DNA, 16 bp, Artificial sequence
  Synthetic oligonucleotide
aaactttagcacctct

SEQ ID NO: 933 - DNA, 16 bp, Artificial sequence
  Synthetic oligonucleotide
ggtcactacacagcaa

SEQ ID NO: 934 - DNA, 16 bp, Artificial sequence
  Synthetic oligonucleotide
tgcacaggcgaaaggt

SEQ ID NO: 935 - DNA, 16 bp, Artificial sequence
  Synthetic oligonucleotide
ttagagttaagtgctg

SEQ ID NO: 936 - DNA, 16 bp, Artificial sequence
  Synthetic oligonucleotide
ccagctgaattaacgc

SEQ ID NO: 937 - DNA, 16 bp, Artificial sequence
  Synthetic oligonucleotide
gtaagggaccctctgc

SEQ ID NO: 938 - DNA, 16 bp, Artificial sequence
  Synthetic oligonucleotide
cattaatagggccacg

SEQ ID NO: 939 - DNA, 16 bp, Artificial sequence
  Synthetic oligonucleotide
ctaagaacctcatgct

SEQ ID NO: 940 - DNA, 16 bp, Artificial sequence
  Synthetic oligonucleotide
accccccatcacaagg

SEQ ID NO: 941 - DNA, 16 bp, Artificial sequence
  Synthetic oligonucleotide
aagatcacacatgggc

SEQ ID NO: 942 - DNA, 16 bp, Artificial sequence
  Synthetic oligonucleotide
gaccacctgacaggca

SEQ ID NO: 943 - DNA, 16 bp, Artificial sequence
  Synthetic oligonucleotide
tagtagtcaaggttat

SEQ ID NO: 944 - DNA, 16 bp, Artificial sequence
  Synthetic oligonucleotide
ggtgaaaaaggtgttc

SEQ ID NO: 945 - DNA, 16 bp, Artificial sequence
  Synthetic oligonucleotide
taaggcttattctact

SEQ ID NO: 946 - DNA, 16 bp, Artificial sequence
  Synthetic oligonucleotide
gaggtgaatgccctgc

SEQ ID NO: 947 - DNA, 16 bp, Artificial sequence
  Synthetic oligonucleotide
gtgtgctccagtggag

SEQ ID NO: 948 - DNA, 16 bp, Artificial sequence
  Synthetic oligonucleotide
ctggtccctgcagaaa

SEQ ID NO: 949 - DNA, 16 bp, Artificial sequence
  Synthetic oligonucleotide
caatgctacttgcccc

SEQ ID NO: 950 - DNA, 16 bp, Artificial sequence
  Synthetic oligonucleotide
taaatcttgggatgca

SEQ ID NO: 951 - DNA, 16 bp, Artificial sequence
  Synthetic oligonucleotide
acaatgacatcatggc

SEQ ID NO: 952 - DNA, 16 bp, Artificial sequence
  Synthetic oligonucleotide
gcaaactgcttacatc

SEQ ID NO: 953 - DNA, 16 bp, Artificial sequence
  Synthetic oligonucleotide
gttagacgcgccaggc

SEQ ID NO: 954 - DNA, 16 bp, Artificial sequence
  Synthetic oligonucleotide
tctcatctattgcata

SEQ ID NO: 955 - DNA, 16 bp, Artificial sequence
  Synthetic oligonucleotide
ttttatttccgttaac

SEQ ID NO: 956 - DNA, 16 bp, Artificial sequence
  Synthetic oligonucleotide
taaactaccgaacgca

SEQ ID NO: 957 - DNA, 16 bp, Artificial sequence
  Synthetic oligonucleotide
cccggaataagcctcc

SEQ ID NO: 958 - DNA, 16 bp, Artificial sequence
  Synthetic oligonucleotide
ctgtacaatgggacgg

SEQ ID NO: 959 - DNA, 16 bp, Artificial sequence
  Synthetic oligonucleotide
tcccatcgacacagca

SEQ ID NO: 960 - DNA, 16 bp, Artificial sequence
  Synthetic oligonucleotide
ggaatattgccaggta

SEQ ID NO: 961 - DNA, 16 bp, Artificial sequence
  Synthetic oligonucleotide
tggtttataacaactg

SEQ ID NO: 962 - DNA, 16 bp, Artificial sequence
  Synthetic oligonucleotide
attaggagaggtctca

SEQ ID NO: 963 - DNA, 16 bp, Artificial sequence
  Synthetic oligonucleotide
cttgatagtgaatgtg

SEQ ID NO: 964 - DNA, 16 bp, Artificial sequence
  Synthetic oligonucleotide
ggcactcacaaaagcg

SEQ ID NO: 965 - DNA, 16 bp, Artificial sequence
  Synthetic oligonucleotide
ccctatgttctacttt

SEQ ID NO: 966 - DNA, 16 bp, Artificial sequence
  Synthetic oligonucleotide
caacatctctagctgg

SEQ ID NO: 967 - DNA, 16 bp, Artificial sequence
  Synthetic oligonucleotide
ggtaatattcagacca

SEQ ID NO: 968 - DNA, 16 bp, Artificial sequence
  Synthetic oligonucleotide
tgaagctacagatcca

SEQ ID NO: 969 - DNA, 16 bp, Artificial sequence
  Synthetic oligonucleotide
ggaaatctgtcagagc

SEQ ID NO: 970 - DNA, 16 bp, Artificial sequence
  Synthetic oligonucleotide
gaatctaggaaggcga

SEQ ID NO: 971 - DNA, 16 bp, Artificial sequence
  Synthetic oligonucleotide
agtatttacctggagg

SEQ ID NO: 972 - DNA, 16 bp, Artificial sequence
  Synthetic oligonucleotide
agccttaggaagcctc

SEQ ID NO: 973 - DNA, 16 bp, Artificial sequence
  Synthetic oligonucleotide
tcggccagggcattcc

SEQ ID NO: 974 - DNA, 16 bp, Artificial sequence
  Synthetic oligonucleotide
cgcagcagctccgccc

SEQ ID NO: 975 - DNA, 16 bp, Artificial sequence
  Synthetic oligonucleotide
cgcgggttaggatctg

SEQ ID NO: 976 - DNA, 16 bp, Artificial sequence
  Synthetic oligonucleotide
gcgggtcgccccgacg

SEQ ID NO: 977 - DNA, 16 bp, Artificial sequence
  Synthetic oligonucleotide
acgccgacgcagtgca

SEQ ID NO: 978 - DNA, 16 bp, Artificial sequence
  Synthetic oligonucleotide
gatctgagaggacctg

SEQ ID NO: 979 - DNA, 16 bp, Artificial sequence
  Synthetic oligonucleotide
caatgttccgactcct

SEQ ID NO: 980 - DNA, 16 bp, Artificial sequence
  Synthetic oligonucleotide
ggaggaacttgcttaa

SEQ ID NO: 981 - DNA, 16 bp, Artificial sequence
  Synthetic oligonucleotide
ggccgggaggcatttg

SEQ ID NO: 982 - DNA, 16 bp, Artificial sequence
  Synthetic oligonucleotide
tattttgccggagatg

SEQ ID NO: 983 - DNA, 16 bp, Artificial sequence
  Synthetic oligonucleotide
acactctggtaagaga

SEQ ID NO: 984 - DNA, 16 bp, Artificial sequence
  Synthetic oligonucleotide
cacggtgatggttgtt

SEQ ID NO: 985 - DNA, 16 bp, Artificial sequence
  Synthetic oligonucleotide
gagcttggtgatgtcc

SEQ ID NO: 986 - DNA, 16 bp, Artificial sequence
  Synthetic oligonucleotide
caaaagctctcgagag

SEQ ID NO: 987 - DNA, 16 bp, Artificial sequence
  Synthetic oligonucleotide
gcatccaaatatcctc

SEQ ID NO: 988 - DNA, 16 bp, Artificial sequence
  Synthetic oligonucleotide
tcaggatccatccctt

SEQ ID NO: 989 - DNA, 16 bp, Artificial sequence
  Synthetic oligonucleotide
cagactcatgtttgcc

SEQ ID NO: 990 - DNA, 16 bp, Artificial sequence
  Synthetic oligonucleotide
gctgagacgcaggtgg

SEQ ID NO: 991 - DNA, 16 bp, Artificial sequence
  Synthetic oligonucleotide
cagtgctgtagcgagc

SEQ ID NO: 992 - DNA, 16 bp, Artificial sequence
  Synthetic oligonucleotide
ctaatgggtagcaagt

SEQ ID NO: 993 - DNA, 16 bp, Artificial sequence
  Synthetic oligonucleotide
gcaatggcagattcca

SEQ ID NO: 994 - DNA, 16 bp, Artificial sequence
  Synthetic oligonucleotide
aggtcacccactgcaa

SEQ ID NO: 995 - DNA, 16 bp, Artificial sequence
  Synthetic oligonucleotide
gacacatcagcactcg

SEQ ID NO: 996 - DNA, 16 bp, Artificial sequence
  Synthetic oligonucleotide
agtcctgctcaggtgt

SEQ ID NO: 997 - DNA, 16 bp, Artificial sequence
  Synthetic oligonucleotide
aagttcaggctggacc

SEQ ID NO: 998 - DNA, 16 bp, Artificial sequence
  Synthetic oligonucleotide
ggtggagagcccctca

SEQ ID NO: 999 - DNA, 16 bp, Artificial sequence
  Synthetic oligonucleotide
ctcgcctcctcaagtg

SEQ ID NO: 1000 - DNA, 16 bp, Artificial sequence
  Synthetic oligonucleotide
gatgggaaactttagc

SEQ ID NO: 1001 - DNA, 16 bp, Artificial sequence
  Synthetic oligonucleotide
gggtcactacacagca

SEQ ID NO: 1002 - DNA, 16 bp, Artificial sequence
  Synthetic oligonucleotide
ctgcacaggcgaaagg

SEQ ID NO: 1003 - DNA, 16 bp, Artificial sequence
  Synthetic oligonucleotide
attagagttaagtgct

SEQ ID NO: 1004 - DNA, 16 bp, Artificial sequence
  Synthetic oligonucleotide
accagctgaattaacg

SEQ ID NO: 1005 - DNA, 16 bp, Artificial sequence
  Synthetic oligonucleotide
gtcagtaagggaccct

SEQ ID NO: 1006 - DNA, 16 bp, Artificial sequence
  Synthetic oligonucleotide
gaccattaatagggcc

SEQ ID NO: 1007 - DNA, 16 bp, Artificial sequence
  Synthetic oligonucleotide
tctaagaacctcatgc

SEQ ID NO: 1008 - DNA, 16 bp, Artificial sequence
  Synthetic oligonucleotide
taccccccatcacaag

SEQ ID NO: 1009 - DNA, 16 bp, Artificial sequence
  Synthetic oligonucleotide
acaagatcacacatgg

SEQ ID NO: 1010 - DNA, 16 bp, Artificial sequence
  Synthetic oligonucleotide
agaccacctgacaggc

SEQ ID NO: 1011 - DNA, 16 bp, Artificial sequence
  Synthetic oligonucleotide
ttagtagtcaaggtta

SEQ ID NO: 1012 - DNA, 16 bp, Artificial sequence
  Synthetic oligonucleotide
aggtgaaaaaggtgtt

SEQ ID NO: 1013 - DNA, 16 bp, Artificial sequence
  Synthetic oligonucleotide
ttaaggcttattctac

SEQ ID NO: 1014 - DNA, 16 bp, Artificial sequence
  Synthetic oligonucleotide
tgaggtgaatgccctg

SEQ ID NO: 1015 - DNA, 16 bp, Artificial sequence
  Synthetic oligonucleotide
tgtgtgctccagtgga

SEQ ID NO: 1016 - DNA, 16 bp, Artificial sequence
  Synthetic oligonucleotide
gctggtccctgcagaa

SEQ ID NO: 1017 - DNA, 16 bp, Artificial sequence
  Synthetic oligonucleotide
gggacgcacgagagtc

SEQ ID NO: 1018 - DNA, 16 bp, Artificial sequence
  Synthetic oligonucleotide
gtcaatagcttcacaa

SEQ ID NO: 1019 - DNA, 16 bp, Artificial sequence
  Synthetic oligonucleotide
ataaatcttgggatgc

SEQ ID NO: 1020 - DNA, 16 bp, Artificial sequence
  Synthetic oligonucleotide
cacaatgacatcatgg

SEQ ID NO: 1021 - DNA, 16 bp, Artificial sequence
  Synthetic oligonucleotide
gataagcaaactgctt

SEQ ID NO: 1022 - DNA, 16 bp, Artificial sequence
  Synthetic oligonucleotide
gaggatgcaactggct

SEQ ID NO: 1023 - DNA, 16 bp, Artificial sequence
  Synthetic oligonucleotide
tcggacttcaggccca

SEQ ID NO: 1024 - DNA, 16 bp, Artificial sequence
  Synthetic oligonucleotide
ccttttatttccgtta

SEQ ID NO: 1025 - DNA, 16 bp, Artificial sequence
  Synthetic oligonucleotide
gcatactaaaaccacc

SEQ ID NO: 1026 - DNA, 16 bp, Artificial sequence
  Synthetic oligonucleotide
gactttgcaggcaccc

SEQ ID NO: 1027 - DNA, 16 bp, Artificial sequence
  Synthetic oligonucleotide
tgacatcccagttcaa

SEQ ID NO: 1028 - DNA, 16 bp, Artificial sequence
  Synthetic oligonucleotide
tactttcccatcgaca

SEQ ID NO: 1029 - DNA, 16 bp, Artificial sequence
  Synthetic oligonucleotide
aggaatattgccaggt

SEQ ID NO: 1030 - DNA, 16 bp, Artificial sequence
  Synthetic oligonucleotide
ggttagtgttggttta

SEQ ID NO: 1031 - DNA, 16 bp, Artificial sequence
  Synthetic oligonucleotide
cattcgatggaggttc

SEQ ID NO: 1032 - DNA, 16 bp, Artificial sequence
  Synthetic oligonucleotide
ggcggatttccccact

SEQ ID NO: 1033 - DNA, 16 bp, Artificial sequence
  Synthetic oligonucleotide
taaaatacgcccgtcc

SEQ ID NO: 1034 - DNA, 16 bp, Artificial sequence
  Synthetic oligonucleotide
tccctatgttctactt

SEQ ID NO: 1035 - DNA, 16 bp, Artificial sequence
  Synthetic oligonucleotide
atcaacatctctagct

SEQ ID NO: 1036 - DNA, 16 bp, Artificial sequence
  Synthetic oligonucleotide
gggtaatattcagacc

SEQ ID NO: 1037 - DNA, 16 bp, Artificial sequence
  Synthetic oligonucleotide
tttactagagactctg

SEQ ID NO: 1038 - DNA, 16 bp, Artificial sequence
  Synthetic oligonucleotide
gtaggataggactaga

SEQ ID NO: 1039 - DNA, 16 bp, Artificial sequence
  Synthetic oligonucleotide
ataaatgcctgaccac

SEQ ID NO: 1040 - DNA, 16 bp, Artificial sequence
  Synthetic oligonucleotide
tgtttctagaatgtcg

SEQ ID NO: 1041 - DNA, 16 bp, Artificial sequence
  Synthetic oligonucleotide
gcctatcagtttcccc

SEQ ID NO: 1042 - DNA, 16 bp, Artificial sequence
  Synthetic oligonucleotide
ctcggccagggcattc

SEQ ID NO: 1043 - DNA, 16 bp, Artificial sequence
  Synthetic oligonucleotide
tccgcagcagctccgc

SEQ ID NO: 1044 - DNA, 16 bp, Artificial sequence
  Synthetic oligonucleotide
gcgcgggttaggatct

SEQ ID NO: 1045 - DNA, 16 bp, Artificial sequence
  Synthetic oligonucleotide
agcgggtcgccccgac

SEQ ID NO: 1046 - DNA, 16 bp, Artificial sequence
  Synthetic oligonucleotide
ataccggagaggacgc

SEQ ID NO: 1047 - DNA, 16 bp, Artificial sequence
  Synthetic oligonucleotide
aagatctgagaggacc

SEQ ID NO: 1048 - DNA, 16 bp, Artificial sequence
  Synthetic oligonucleotide
ccaatgttccgactcc

SEQ ID NO: 1049 - DNA, 16 bp, Artificial sequence
  Synthetic oligonucleotide
cggaggaacttgctta

SEQ ID NO: 1050 - DNA, 16 bp, Artificial sequence
  Synthetic oligonucleotide
tggccgggaggcattt

SEQ ID NO: 1051 - DNA, 16 bp, Artificial sequence
  Synthetic oligonucleotide
ctattttgccggagat

SEQ ID NO: 1052 - DNA, 16 bp, Artificial sequence
  Synthetic oligonucleotide
gacactctggtaagag

SEQ ID NO: 1053 - DNA, 16 bp, Artificial sequence
  Synthetic oligonucleotide
acacggtgatggttgt

SEQ ID NO: 1054 - DNA, 16 bp, Artificial sequence
  Synthetic oligonucleotide
actgagcttggtgatg

SEQ ID NO: 1055 - DNA, 16 bp, Artificial sequence
  Synthetic oligonucleotide
acaaaagctctcgaga

SEQ ID NO: 1056 - DNA, 16 bp, Artificial sequence
  Synthetic oligonucleotide
acctgaatgcatccaa

SEQ ID NO: 1057 - DNA, 16 bp, Artificial sequence
  Synthetic oligonucleotide
ctcaggatccatccct

SEQ ID NO: 1058 - DNA, 16 bp, Artificial sequence
  Synthetic oligonucleotide
ccagactcatgtttgc

SEQ ID NO: 1059 - DNA, 16 bp, Artificial sequence
  Synthetic oligonucleotide
tgctgagacgcaggtg

SEQ ID NO: 1060 - DNA, 16 bp, Artificial sequence
  Synthetic oligonucleotide
tcagtgctgtagcgag

SEQ ID NO: 1061 - DNA, 16 bp, Artificial sequence
  Synthetic oligonucleotide
tatcctaatgggtagc

SEQ ID NO: 1062 - DNA, 16 bp, Artificial sequence
  Synthetic oligonucleotide
tcgcaatggcagattc

SEQ ID NO: 1063 - DNA, 16 bp, Artificial sequence
  Synthetic oligonucleotide
tgtgaggtcacccact

SEQ ID NO: 1064 - DNA, 16 bp, Artificial sequence
  Synthetic oligonucleotide
agacacatcagcactc

SEQ ID NO: 1065 - DNA, 16 bp, Artificial sequence
  Synthetic oligonucleotide
cagtcctgctcaggtg

SEQ ID NO: 1066 - DNA, 16 bp, Artificial sequence
  Synthetic oligonucleotide
gaagttcaggctggac

SEQ ID NO: 1067 - DNA, 16 bp, Artificial sequence
  Synthetic oligonucleotide
aggtggagagcccctc

SEQ ID NO: 1068 - DNA, 16 bp, Artificial sequence
  Synthetic oligonucleotide
actcgcctcctcaagt

SEQ ID NO: 1069 - DNA, 16 bp, Artificial sequence
  Synthetic oligonucleotide
agatgggaaactttag

SEQ ID NO: 1070 - DNA, 16 bp, Artificial sequence
  Synthetic oligonucleotide
ggctgggatcctccac

SEQ ID NO: 1071 - DNA, 16 bp, Artificial sequence
  Synthetic oligonucleotide
gctgcacaggcgaaag

SEQ ID NO: 1072 - DNA, 16 bp, Artificial sequence
  Synthetic oligonucleotide
tattagagttaagtgc

SEQ ID NO: 1073 - DNA, 16 bp, Artificial sequence
  Synthetic oligonucleotide
aaccagctgaattaac

SEQ ID NO: 1074 - DNA, 16 bp, Artificial sequence
  Synthetic oligonucleotide
cagtcagtaagggacc

SEQ ID NO: 1075 - DNA, 16 bp, Artificial sequence
  Synthetic oligonucleotide
tgaccattaatagggc

SEQ ID NO: 1076 - DNA, 16 bp, Artificial sequence
  Synthetic oligonucleotide
ttctaagaacctcatg

SEQ ID NO: 1077 - DNA, 16 bp, Artificial sequence
  Synthetic oligonucleotide
ctaccccccatcacaa

SEQ ID NO: 1078 - DNA, 16 bp, Artificial sequence
  Synthetic oligonucleotide
ccacaagatcacacat

SEQ ID NO: 1079 - DNA, 16 bp, Artificial sequence
  Synthetic oligonucleotide
cagaccacctgacagg

SEQ ID NO: 1080 - DNA, 16 bp, Artificial sequence
  Synthetic oligonucleotide
tttagtagtcaaggtt

SEQ ID NO: 1081 - DNA, 16 bp, Artificial sequence
  Synthetic oligonucleotide
taggtgaaaaaggtgt

SEQ ID NO: 1082 - DNA, 16 bp, Artificial sequence
  Synthetic oligonucleotide
acccaaccgatttttt

SEQ ID NO: 1083 - DNA, 16 bp, Artificial sequence
  Synthetic oligonucleotide
ctgaggtgaatgccct

SEQ ID NO: 1084 - DNA, 16 bp, Artificial sequence
  Synthetic oligonucleotide
ttgtgtgctccagtgg

SEQ ID NO: 1085 - DNA, 16 bp, Artificial sequence
  Synthetic oligonucleotide
tcactgaccatgtggg

SEQ ID NO: 1086 - DNA, 16 bp, Artificial sequence
  Synthetic oligonucleotide
cttcatgcacgggcgc

SEQ ID NO: 1087 - DNA, 16 bp, Artificial sequence
  Synthetic oligonucleotide
gcataatctcctgcct

SEQ ID NO: 1088 - DNA, 16 bp, Artificial sequence
  Synthetic oligonucleotide
gccataaatcttggga

SEQ ID NO: 1089 - DNA, 16 bp, Artificial sequence
  Synthetic oligonucleotide
ctttattcaatgtggc

SEQ ID NO: 1090 - DNA, 16 bp, Artificial sequence
  Synthetic oligonucleotide
tacaactgcctgtgtt

SEQ ID NO: 1091 - DNA, 16 bp, Artificial sequence
  Synthetic oligonucleotide
aaagcttccgcaaaca

SEQ ID NO: 1092 - DNA, 16 bp, Artificial sequence
  Synthetic oligonucleotide
ctaacatacaccctcg

SEQ ID NO: 1093 - DNA, 16 bp, Artificial sequence
  Synthetic oligonucleotide
agcttctgggacaagc

SEQ ID NO: 1094 - DNA, 16 bp, Artificial sequence
  Synthetic oligonucleotide
ggcatactaaaaccac

SEQ ID NO: 1095 - DNA, 16 bp, Artificial sequence
  Synthetic oligonucleotide
ttgaatgtcacccttc

SEQ ID NO: 1096 - DNA, 16 bp, Artificial sequence
  Synthetic oligonucleotide
agtcatgacatcccag

SEQ ID NO: 1097 - DNA, 16 bp, Artificial sequence
  Synthetic oligonucleotide
tctcattggcacctgt

SEQ ID NO: 1098 - DNA, 16 bp, Artificial sequence
  Synthetic oligonucleotide
ccctatcagatgccct

SEQ ID NO: 1099 - DNA, 16 bp, Artificial sequence
  Synthetic oligonucleotide
catatctggtttcatg

SEQ ID NO: 1100 - DNA, 16 bp, Artificial sequence
  Synthetic oligonucleotide
gaccatagcactgtct

SEQ ID NO: 1101 - DNA, 16 bp, Artificial sequence
  Synthetic oligonucleotide
attaatctggtcatat

SEQ ID NO: 1102 - DNA, 16 bp, Artificial sequence
  Synthetic oligonucleotide
tccataaaatacgccc

SEQ ID NO: 1103 - DNA, 16 bp, Artificial sequence
  Synthetic oligonucleotide
gaaagatggaattccc

SEQ ID NO: 1104 - DNA, 16 bp, Artificial sequence
  Synthetic oligonucleotide
tacgatcatcattatt

SEQ ID NO: 1105 - DNA, 16 bp, Artificial sequence
  Synthetic oligonucleotide
gtattagctcaatatt

SEQ ID NO: 1106 - DNA, 16 bp, Artificial sequence
  Synthetic oligonucleotide
gtttactagagactct

SEQ ID NO: 1107 - DNA, 16 bp, Artificial sequence
  Synthetic oligonucleotide
gtaaaaactgtaggat

SEQ ID NO: 1108 - DNA, 16 bp, Artificial sequence
  Synthetic oligonucleotide
gataaatgcctgacca

SEQ ID NO: 1109 - DNA, 16 bp, Artificial sequence
  Synthetic oligonucleotide
ccgacgggaagtcttc

SEQ ID NO: 1110 - DNA, 16 bp, Artificial sequence
  Synthetic oligonucleotide
ggcctatcagtttccc

SEQ ID NO: 1111 - DNA, 16 bp, Artificial sequence
  Synthetic oligonucleotide
tctcggccagggcatt

SEQ ID NO: 1112 - DNA, 16 bp, Artificial sequence
  Synthetic oligonucleotide
atccgcagcagctccg

SEQ ID NO: 1113 - DNA, 16 bp, Artificial sequence
  Synthetic oligonucleotide
ggcgcgggttaggatc

SEQ ID NO: 1114 - DNA, 16 bp, Artificial sequence
  Synthetic oligonucleotide
cagcgggtcgccccga

SEQ ID NO: 1115 - DNA, 16 bp, Artificial sequence
  Synthetic oligonucleotide
gataccggagaggacg

SEQ ID NO: 1116 - DNA, 16 bp, Artificial sequence
  Synthetic oligonucleotide
gcacaagatctgagag

SEQ ID NO: 1117 - DNA, 16 bp, Artificial sequence
  Synthetic oligonucleotide
tgccaatgttccgact

SEQ ID NO: 1118 - DNA, 16 bp, Artificial sequence
  Synthetic oligonucleotide
tcggaggaacttgctt

SEQ ID NO: 1119 - DNA, 16 bp, Artificial sequence
  Synthetic oligonucleotide
ttggccgggaggcatt

SEQ ID NO: 1120 - DNA, 16 bp, Artificial sequence
  Synthetic oligonucleotide
cctattttgccggaga

SEQ ID NO: 1121 - DNA, 16 bp, Artificial sequence
  Synthetic oligonucleotide
agacactctggtaaga

SEQ ID NO: 1122 - DNA, 16 bp, Artificial sequence
  Synthetic oligonucleotide
gacacggtgatggttg

SEQ ID NO: 1123 - DNA, 16 bp, Artificial sequence
  Synthetic oligonucleotide
gactgagcttggtgat

SEQ ID NO: 1124 - DNA, 16 bp, Artificial sequence
  Synthetic oligonucleotide
gacaaaagctctcgag

SEQ ID NO: 1125 - DNA, 16 bp, Artificial sequence
  Synthetic oligonucleotide
aacctgaatgcatcca

SEQ ID NO: 1126 - DNA, 16 bp, Artificial sequence
  Synthetic oligonucleotide
cctcaggatccatccc

SEQ ID NO: 1127 - DNA, 16 bp, Artificial sequence
  Synthetic oligonucleotide
aatccagactcatgtt

SEQ ID NO: 1128 - DNA, 16 bp, Artificial sequence
  Synthetic oligonucleotide
caggatgctgagacgc

SEQ ID NO: 1129 - DNA, 16 bp, Artificial sequence
  Synthetic oligonucleotide
ctcagtgctgtagcga

SEQ ID NO: 1130 - DNA, 16 bp, Artificial sequence
  Synthetic oligonucleotide
ttatcctaatgggtag

SEQ ID NO: 1131 - DNA, 16 bp, Artificial sequence
  Synthetic oligonucleotide
atcgcaatggcagatt

SEQ ID NO: 1132 - DNA, 16 bp, Artificial sequence
  Synthetic oligonucleotide
cacctgtgaggtcacc

SEQ ID NO: 1133 - DNA, 16 bp, Artificial sequence
  Synthetic oligonucleotide
cagacacatcagcact

SEQ ID NO: 1134 - DNA, 16 bp, Artificial sequence
  Synthetic oligonucleotide
ccagtcctgctcaggt

SEQ ID NO: 1135 - DNA, 16 bp, Artificial sequence
  Synthetic oligonucleotide
agaagaagttcaggct

SEQ ID NO: 1136 - DNA, 16 bp, Artificial sequence
  Synthetic oligonucleotide
aaaggtggagagcccc

SEQ ID NO: 1137 - DNA, 16 bp, Artificial sequence
  Synthetic oligonucleotide
gactcgcctcctcaag

SEQ ID NO: 1138 - DNA, 16 bp, Artificial sequence
  Synthetic oligonucleotide
ggtagctgcacaaaga

SEQ ID NO: 1139 - DNA, 16 bp, Artificial sequence
  Synthetic oligonucleotide
gaggctgggatcctcc

SEQ ID NO: 1140 - DNA, 16 bp, Artificial sequence
  Synthetic oligonucleotide
cgctgcacaggcgaaa

SEQ ID NO: 1141 - DNA, 16 bp, Artificial sequence
  Synthetic oligonucleotide
gatgtattagagttaa

SEQ ID NO: 1142 - DNA, 16 bp, Artificial sequence
  Synthetic oligonucleotide
caaccagctgaattaa

SEQ ID NO: 1143 - DNA, 16 bp, Artificial sequence
  Synthetic oligonucleotide
acagtcagtaagggac

SEQ ID NO: 1144 - DNA, 16 bp, Artificial sequence
  Synthetic oligonucleotide
ctgaccattaataggg

SEQ ID NO: 1145 - DNA, 16 bp, Artificial sequence
  Synthetic oligonucleotide
gtcattctaagaacct

SEQ ID NO: 1146 - DNA, 16 bp, Artificial sequence
  Synthetic oligonucleotide
cctaccccccatcaca

SEQ ID NO: 1147 - DNA, 16 bp, Artificial sequence
  Synthetic oligonucleotide
accccacaagatcaca

SEQ ID NO: 1148 - DNA, 16 bp, Artificial sequence
  Synthetic oligonucleotide
gcagaccacctgacag

SEQ ID NO: 1149 - DNA, 16 bp, Artificial sequence
  Synthetic oligonucleotide
ccccgccatggagacg

SEQ ID NO: 1150 - DNA, 16 bp, Artificial sequence
  Synthetic oligonucleotide
gttaggtgaaaaaggt

SEQ ID NO: 1151 - DNA, 16 bp, Artificial sequence
  Synthetic oligonucleotide
gcacccaaccgatttt

SEQ ID NO: 1152 - DNA, 16 bp, Artificial sequence
  Synthetic oligonucleotide
gctgaggtgaatgccc

SEQ ID NO: 1153 - DNA, 16 bp, Artificial sequence
  Synthetic oligonucleotide
gttgtgtgctccagtg

SEQ ID NO: 1154 - DNA, 16 bp, Artificial sequence
  Synthetic oligonucleotide
ctcactgaccatgtgg

SEQ ID NO: 1155 - DNA, 16 bp, Artificial sequence
  Synthetic oligonucleotide
gcaaatcggcccctcg

SEQ ID NO: 1156 - DNA, 16 bp, Artificial sequence
  Synthetic oligonucleotide
ggcataatctcctgcc

SEQ ID NO: 1157 - DNA, 16 bp, Artificial sequence
  Synthetic oligonucleotide
tggcatgcaagaccac

SEQ ID NO: 1158 - DNA, 16 bp, Artificial sequence
  Synthetic oligonucleotide
actttattcaatgtgg

SEQ ID NO: 1159 - DNA, 16 bp, Artificial sequence
  Synthetic oligonucleotide
gtttatgtcactctgg

SEQ ID NO: 1160 - DNA, 16 bp, Artificial sequence
  Synthetic oligonucleotide
gaacagacaagtgctg

SEQ ID NO: 1161 - DNA, 16 bp, Artificial sequence
  Synthetic oligonucleotide
actaacatacaccctc

SEQ ID NO: 1162 - DNA, 16 bp, Artificial sequence
  Synthetic oligonucleotide
ataatcagggtggtgc

SEQ ID NO: 1163 - DNA, 16 bp, Artificial sequence
  Synthetic oligonucleotide
aggcatactaaaacca

SEQ ID NO: 1164 - DNA, 16 bp, Artificial sequence
  Synthetic oligonucleotide
gaatcatgcaagctct

SEQ ID NO: 1165 - DNA, 16 bp, Artificial sequence
  Synthetic oligonucleotide
taaactaagggtcaca

SEQ ID NO: 1166 - DNA, 16 bp, Artificial sequence
  Synthetic oligonucleotide
atccatcctgcatgag

SEQ ID NO: 1167 - DNA, 16 bp, Artificial sequence
  Synthetic oligonucleotide
ggccctatcagatgcc

SEQ ID NO: 1168 - DNA, 16 bp, Artificial sequence
  Synthetic oligonucleotide
ctacatatctggtttc

SEQ ID NO: 1169 - DNA, 16 bp, Artificial sequence
  Synthetic oligonucleotide
tggaccatagcactgt

SEQ ID NO: 1170 - DNA, 16 bp, Artificial sequence
  Synthetic oligonucleotide
tattaatctggtcata

SEQ ID NO: 1171 - DNA, 16 bp, Artificial sequence
  Synthetic oligonucleotide
ccactttactctgttg

SEQ ID NO: 1172 - DNA, 16 bp, Artificial sequence
  Synthetic oligonucleotide
aactatgcctagaacg

SEQ ID NO: 1173 - DNA, 16 bp, Artificial sequence
  Synthetic oligonucleotide
tttacgatcatcatta

SEQ ID NO: 1174 - DNA, 16 bp, Artificial sequence
  Synthetic oligonucleotide
tgtattagctcaatat

SEQ ID NO: 1175 - DNA, 16 bp, Artificial sequence
  Synthetic oligonucleotide
tgggagtttactagag

SEQ ID NO: 1176 - DNA, 16 bp, Artificial sequence
  Synthetic oligonucleotide
agagagtactcttgga

SEQ ID NO: 1177 - DNA, 16 bp, Artificial sequence
  Synthetic oligonucleotide
ctgataaatgcctgac

SEQ ID NO: 1178 - DNA, 16 bp, Artificial sequence
  Synthetic oligonucleotide
atcaatgctgcactca

SEQ ID NO: 1179 - DNA, 16 bp, Artificial sequence
  Synthetic oligonucleotide
acgaatccctggaggg

SEQ ID NO: 1180 - DNA, 16 bp, Artificial sequence
  Synthetic oligonucleotide
gtctcggccagggcat

SEQ ID NO: 1181 - DNA, 16 bp, Artificial sequence
  Synthetic oligonucleotide
ctgatccgcagcagct

SEQ ID NO: 1182 - DNA, 16 bp, Artificial sequence
  Synthetic oligonucleotide
cgtacatggcggcggc

SEQ ID NO: 1183 - DNA, 16 bp, Artificial sequence
  Synthetic oligonucleotide
gcagcgggtcgccccg

SEQ ID NO: 1184 - DNA, 16 bp, Artificial sequence
  Synthetic oligonucleotide
ggataccggagaggac

SEQ ID NO: 1185 - DNA, 16 bp, Artificial sequence
  Synthetic oligonucleotide
cgcacaagatctgaga

SEQ ID NO: 1186 - DNA, 16 bp, Artificial sequence
  Synthetic oligonucleotide
atgccaatgttccgac

SEQ ID NO: 1187 - DNA, 16 bp, Artificial sequence
  Synthetic oligonucleotide
gtcggaggaacttgct

SEQ ID NO: 1188 - DNA, 16 bp, Artificial sequence
  Synthetic oligonucleotide
attggccgggaggcat

SEQ ID NO: 1189 - DNA, 16 bp, Artificial sequence
  Synthetic oligonucleotide
gcctattttgccggag

SEQ ID NO: 1190 - DNA, 16 bp, Artificial sequence
  Synthetic oligonucleotide
cagacactctggtaag

SEQ ID NO: 1191 - DNA, 16 bp, Artificial sequence
  Synthetic oligonucleotide
tactccccatagaagg

SEQ ID NO: 1192 - DNA, 16 bp, Artificial sequence
  Synthetic oligonucleotide
tagactgagcttggtg

SEQ ID NO: 1193 - DNA, 16 bp, Artificial sequence
  Synthetic oligonucleotide
ggacaaaagctctcga

SEQ ID NO: 1194 - DNA, 16 bp, Artificial sequence
  Synthetic oligonucleotide
gaacctgaatgcatcc

SEQ ID NO: 1195 - DNA, 16 bp, Artificial sequence
  Synthetic oligonucleotide
cgacctcaggatccat

SEQ ID NO: 1196 - DNA, 16 bp, Artificial sequence
  Synthetic oligonucleotide
gaatccagactcatgt

SEQ ID NO: 1197 - DNA, 16 bp, Artificial sequence
  Synthetic oligonucleotide
gcaggatgctgagacg

SEQ ID NO: 1198 - DNA, 16 bp, Artificial sequence
  Synthetic oligonucleotide
actcagtgctgtagcg

SEQ ID NO: 1199 - DNA, 16 bp, Artificial sequence
  Synthetic oligonucleotide
attatcctaatgggta

SEQ ID NO: 1200 - DNA, 16 bp, Artificial sequence
  Synthetic oligonucleotide
aatcgcaatggcagat

SEQ ID NO: 1201 - DNA, 16 bp, Artificial sequence
  Synthetic oligonucleotide
aacacctgtgaggtca

SEQ ID NO: 1202 - DNA, 16 bp, Artificial sequence
  Synthetic oligonucleotide
ggagcagacacatcag

SEQ ID NO: 1203 - DNA, 16 bp, Artificial sequence
  Synthetic oligonucleotide
gccagtcctgctcagg

SEQ ID NO: 1204 - DNA, 16 bp, Artificial sequence
  Synthetic oligonucleotide
aagaagaagttcaggc

SEQ ID NO: 1205 - DNA, 16 bp, Artificial sequence
  Synthetic oligonucleotide
gaaaggtggagagccc

SEQ ID NO: 1206 - DNA, 16 bp, Artificial sequence
  Synthetic oligonucleotide
tagactcgcctcctca

SEQ ID NO: 1207 - DNA, 16 bp, Artificial sequence
  Synthetic oligonucleotide
gaggtagctgcacaaa

SEQ ID NO: 1208 - DNA, 16 bp, Artificial sequence
  Synthetic oligonucleotide
aactcagctcagaggc

SEQ ID NO: 1209 - DNA, 16 bp, Artificial sequence
  Synthetic oligonucleotide
ccgctgcacaggcgaa

SEQ ID NO: 1210 - DNA, 16 bp, Artificial sequence
  Synthetic oligonucleotide
ctgatgtattagagtt

SEQ ID NO: 1211 - DNA, 16 bp, Artificial sequence
  Synthetic oligonucleotide
ccaaccagctgaatta

SEQ ID NO: 1212 - DNA, 16 bp, Artificial sequence
  Synthetic oligonucleotide
aacagtcagtaaggga

SEQ ID NO: 1213 - DNA, 16 bp, Artificial sequence
  Synthetic oligonucleotide
tctgaccattaatagg

SEQ ID NO: 1214 - DNA, 16 bp, Artificial sequence
  Synthetic oligonucleotide
tgtcattctaagaacc

SEQ ID NO: 1215 - DNA, 16 bp, Artificial sequence
  Synthetic oligonucleotide
gcctaccccccatcac

SEQ ID NO: 1216 - DNA, 16 bp, Artificial sequence
  Synthetic oligonucleotide
caccccacaagatcac

SEQ ID NO: 1217 - DNA, 16 bp, Artificial sequence
  Synthetic oligonucleotide
tgcagaccacctgaca

SEQ ID NO: 1218 - DNA, 16 bp, Artificial sequence
  Synthetic oligonucleotide
cccccgccatggagac

SEQ ID NO: 1219 - DNA, 16 bp, Artificial sequence
  Synthetic oligonucleotide
cgcttccttacatttt

SEQ ID NO: 1220 - DNA, 16 bp, Artificial sequence
  Synthetic oligonucleotide
tgcacccaaccgattt

SEQ ID NO: 1221 - DNA, 16 bp, Artificial sequence
  Synthetic oligonucleotide
ggctgaggtgaatgcc

SEQ ID NO: 1222 - DNA, 16 bp, Artificial sequence
  Synthetic oligonucleotide
agttgtgtgctccagt

SEQ ID NO: 1223 - DNA, 16 bp, Artificial sequence
  Synthetic oligonucleotide
actcactgaccatgtg

SEQ ID NO: 1224 - DNA, 16 bp, Artificial sequence
  Synthetic oligonucleotide
ggccaaagccccactc

SEQ ID NO: 1225 - DNA, 16 bp, Artificial sequence
  Synthetic oligonucleotide
gggcataatctcctgc

SEQ ID NO: 1226 - DNA, 16 bp, Artificial sequence
  Synthetic oligonucleotide
ggctgatctgcactct

SEQ ID NO: 1227 - DNA, 16 bp, Artificial sequence
  Synthetic oligonucleotide
taattctacctgtgtc

SEQ ID NO: 1228 - DNA, 16 bp, Artificial sequence
  Synthetic oligonucleotide
agtttatgtcactctg

SEQ ID NO: 1229 - DNA, 16 bp, Artificial sequence
  Synthetic oligonucleotide
acactttgcgaagcac

SEQ ID NO: 1230 - DNA, 16 bp, Artificial sequence
  Synthetic oligonucleotide
gaactaacatacaccc

SEQ ID NO: 1231 - DNA, 16 bp, Artificial sequence
  Synthetic oligonucleotide
cccataatcagggtgg

SEQ ID NO: 1232 - DNA, 16 bp, Artificial sequence
  Synthetic oligonucleotide
gtagagtggtaaggca

SEQ ID NO: 1233 - DNA, 16 bp, Artificial sequence
  Synthetic oligonucleotide
aagaatcatgcaagct

SEQ ID NO: 1234 - DNA, 16 bp, Artificial sequence
  Synthetic oligonucleotide
ttaaactaagggtcac

SEQ ID NO: 1235 - DNA, 16 bp, Artificial sequence
  Synthetic oligonucleotide
ttaatgtggattcacg

SEQ ID NO: 1236 - DNA, 16 bp, Artificial sequence
  Synthetic oligonucleotide
ccaagataacctcaca

SEQ ID NO: 1237 - DNA, 16 bp, Artificial sequence
  Synthetic oligonucleotide
ccatctacatatctgg

SEQ ID NO: 1238 - DNA, 16 bp, Artificial sequence
  Synthetic oligonucleotide
cacaatcatttggacc

SEQ ID NO: 1239 - DNA, 16 bp, Artificial sequence
  Synthetic oligonucleotide
gtattaatctggtcat

SEQ ID NO: 1240 - DNA, 16 bp, Artificial sequence
  Synthetic oligonucleotide
cacctctggacaatcg

SEQ ID NO: 1241 - DNA, 16 bp, Artificial sequence
  Synthetic oligonucleotide
caaactatgcctagaa

SEQ ID NO: 1242 - DNA, 16 bp, Artificial sequence
  Synthetic oligonucleotide
attttacgatcatcat

SEQ ID NO: 1243 - DNA, 16 bp, Artificial sequence
  Synthetic oligonucleotide
tgcctgtattagctca

SEQ ID NO: 1244 - DNA, 16 bp, Artificial sequence
  Synthetic oligonucleotide
cacataaagtcaaacg

SEQ ID NO: 1245 - DNA, 16 bp, Artificial sequence
  Synthetic oligonucleotide
agaacaagagagtact

SEQ ID NO: 1246 - DNA, 16 bp, Artificial sequence
  Synthetic oligonucleotide
cacataaaggaccccc

SEQ ID NO: 1247 - DNA, 16 bp, Artificial sequence
  Synthetic oligonucleotide
cgctatctgacactcc

SEQ ID NO: 1248 - DNA, 16 bp, Artificial sequence
  Synthetic oligonucleotide
tccaccaacgaatccc

SEQ ID NO: 1249 - DNA, 16 bp, Artificial sequence
  Synthetic oligonucleotide
tgtctcggccagggca

SEQ ID NO: 1250 - DNA, 16 bp, Artificial sequence
  Synthetic oligonucleotide
cctgatccgcagcagc

SEQ ID NO: 1251 - DNA, 16 bp, Artificial sequence
  Synthetic oligonucleotide
ccagccgcgctctgcg

SEQ ID NO: 1252 - DNA, 16 bp, Artificial sequence
  Synthetic oligonucleotide
ggcagcgggtcgcccc

SEQ ID NO: 1253 - DNA, 16 bp, Artificial sequence
  Synthetic oligonucleotide
cgggataccggagagg

SEQ ID NO: 1254 - DNA, 16 bp, Artificial sequence
  Synthetic oligonucleotide
ccgcacaagatctgag

SEQ ID NO: 1255 - DNA, 16 bp, Artificial sequence
  Synthetic oligonucleotide
gatgccaatgttccga

SEQ ID NO: 1256 - DNA, 16 bp, Artificial sequence
  Synthetic oligonucleotide
tgtcggaggaacttgc

SEQ ID NO: 1257 - DNA, 16 bp, Artificial sequence
  Synthetic oligonucleotide
cattggccgggaggca

SEQ ID NO: 1258 - DNA, 16 bp, Artificial sequence
  Synthetic oligonucleotide
tgcctattttgccgga

SEQ ID NO: 1259 - DNA, 16 bp, Artificial sequence
  Synthetic oligonucleotide
tcagacactctggtaa

SEQ ID NO: 1260 - DNA, 16 bp, Artificial sequence
  Synthetic oligonucleotide
cgtactccccatagaa

SEQ ID NO: 1261 - DNA, 16 bp, Artificial sequence
  Synthetic oligonucleotide
cgtagactgagcttgg

SEQ ID NO: 1262 - DNA, 16 bp, Artificial sequence
  Synthetic oligonucleotide
caccttgagatccggg

SEQ ID NO: 1263 - DNA, 16 bp, Artificial sequence
  Synthetic oligonucleotide
agaacctgaatgcatc

SEQ ID NO: 1264 - DNA, 16 bp, Artificial sequence
  Synthetic oligonucleotide
gcgacctcaggatcca

SEQ ID NO: 1265 - DNA, 16 bp, Artificial sequence
  Synthetic oligonucleotide
ggcagccgactccggg

SEQ ID NO: 1266 - DNA, 16 bp, Artificial sequence
  Synthetic oligonucleotide
caggatgctctcatcc

SEQ ID NO: 1267 - DNA, 16 bp, Artificial sequence
  Synthetic oligonucleotide
cactcagtgctgtagc

SEQ ID NO: 1268 - DNA, 16 bp, Artificial sequence
  Synthetic oligonucleotide
agacattatcctaatg

SEQ ID NO: 1269 - DNA, 16 bp, Artificial sequence
  Synthetic oligonucleotide
caatcgcaatggcaga

SEQ ID NO: 1270 - DNA, 16 bp, Artificial sequence
  Synthetic oligonucleotide
gaacacctgtgaggtc

SEQ ID NO: 1271 - DNA, 16 bp, Artificial sequence
  Synthetic oligonucleotide
ggctgctcactggcat

SEQ ID NO: 1272 - DNA, 16 bp, Artificial sequence
  Synthetic oligonucleotide
ggccagtcctgctcag

SEQ ID NO: 1273 - DNA, 16 bp, Artificial sequence
  Synthetic oligonucleotide
cccaagaagaagttca

SEQ ID NO: 1274 - DNA, 16 bp, Artificial sequence
  Synthetic oligonucleotide
ggaaaggtggagagcc

SEQ ID NO: 1275 - DNA, 16 bp, Artificial sequence
  Synthetic oligonucleotide
ctagactcgcctcctc

SEQ ID NO: 1276 - DNA, 16 bp, Artificial sequence
  Synthetic oligonucleotide
gcggaggtagctgcac

SEQ ID NO: 1277 - DNA, 16 bp, Artificial sequence
  Synthetic oligonucleotide
caactcagctcagagg

SEQ ID NO: 1278 - DNA, 16 bp, Artificial sequence
  Synthetic oligonucleotide
accgctgcacaggcga

SEQ ID NO: 1279 - DNA, 16 bp, Artificial sequence
  Synthetic oligonucleotide
tgctgatgtattagag

SEQ ID NO: 1280 - DNA, 16 bp, Artificial sequence
  Synthetic oligonucleotide
cccaaccagctgaatt

SEQ ID NO: 1281 - DNA, 16 bp, Artificial sequence
  Synthetic oligonucleotide
aaacagtcagtaaggg

SEQ ID NO: 1282 - DNA, 16 bp, Artificial sequence
  Synthetic oligonucleotide
gtctgaccattaatag

SEQ ID NO: 1283 - DNA, 16 bp, Artificial sequence
  Synthetic oligonucleotide
ctgtcattctaagaac

SEQ ID NO: 1284 - DNA, 16 bp, Artificial sequence
  Synthetic oligonucleotide
agcctaccccccatca

SEQ ID NO: 1285 - DNA, 16 bp, Artificial sequence
  Synthetic oligonucleotide
ccaccccacaagatca

SEQ ID NO: 1286 - DNA, 16 bp, Artificial sequence
  Synthetic oligonucleotide
ttgcagaccacctgac

SEQ ID NO: 1287 - DNA, 16 bp, Artificial sequence
  Synthetic oligonucleotide
acccccgccatggaga

SEQ ID NO: 1288 - DNA, 16 bp, Artificial sequence
  Synthetic oligonucleotide
acgcttccttacattt

SEQ ID NO: 1289 - DNA, 16 bp, Artificial sequence
  Synthetic oligonucleotide
ctgcacccaaccgatt

SEQ ID NO: 1290 - DNA, 16 bp, Artificial sequence
  Synthetic oligonucleotide
gggctgaggtgaatgc

SEQ ID NO: 1291 - DNA, 16 bp, Artificial sequence
  Synthetic oligonucleotide
aagttgtgtgctccag

SEQ ID NO: 1292 - DNA, 16 bp, Artificial sequence
  Synthetic oligonucleotide
gaaactcactgaccat

SEQ ID NO: 1293 - DNA, 16 bp, Artificial sequence
  Synthetic oligonucleotide
ggaaacaactttcctc

SEQ ID NO: 1294 - DNA, 16 bp, Artificial sequence
  Synthetic oligonucleotide
gatcatgtggcggtct

SEQ ID NO: 1295 - DNA, 16 bp, Artificial sequence
  Synthetic oligonucleotide
cacttactggcctggc

SEQ ID NO: 1296 - DNA, 16 bp, Artificial sequence
  Synthetic oligonucleotide
atattgggctcaatga

SEQ ID NO: 1297 - DNA, 16 bp, Artificial sequence
  Synthetic oligonucleotide
atcactggaggtgtac

SEQ ID NO: 1298 - DNA, 16 bp, Artificial sequence
  Synthetic oligonucleotide
caggatcacactttgc

SEQ ID NO: 1299 - DNA, 16 bp, Artificial sequence
  Synthetic oligonucleotide
ggaactaacatacacc

SEQ ID NO: 1300 - DNA, 16 bp, Artificial sequence
  Synthetic oligonucleotide
gtatatgttcccaggt

SEQ ID NO: 1301 - DNA, 16 bp, Artificial sequence
  Synthetic oligonucleotide
gtgtacatggtctgca

SEQ ID NO: 1302 - DNA, 16 bp, Artificial sequence
  Synthetic oligonucleotide
atcattggaagaccgc

SEQ ID NO: 1303 - DNA, 16 bp, Artificial sequence
  Synthetic oligonucleotide
gttaaactaagggtca

SEQ ID NO: 1304 - DNA, 16 bp, Artificial sequence
  Synthetic oligonucleotide
cttaatgtggattcac

SEQ ID NO: 1305 - DNA, 16 bp, Artificial sequence
  Synthetic oligonucleotide
tccaacttcaggctga

SEQ ID NO: 1306 - DNA, 16 bp, Artificial sequence
  Synthetic oligonucleotide
agctttgtgggctcca

SEQ ID NO: 1307 - DNA, 16 bp, Artificial sequence
  Synthetic oligonucleotide
gtttaataagggcacc

SEQ ID NO: 1308 - DNA, 16 bp, Artificial sequence
  Synthetic oligonucleotide
cgtattaatctggtca

SEQ ID NO: 1309 - DNA, 16 bp, Artificial sequence
  Synthetic oligonucleotide
cacctaaaatgctcac

SEQ ID NO: 1310 - DNA, 16 bp, Artificial sequence
  Synthetic oligonucleotide
acaaactatgcctaga

SEQ ID NO: 1311 - DNA, 16 bp, Artificial sequence
  Synthetic oligonucleotide
aattttacgatcatca

SEQ ID NO: 1312 - DNA, 16 bp, Artificial sequence
  Synthetic oligonucleotide
gacagatcagcactcg

SEQ ID NO: 1313 - DNA, 16 bp, Artificial sequence
  Synthetic oligonucleotide
caattctagacatggc

SEQ ID NO: 1314 - DNA, 16 bp, Artificial sequence
  Synthetic oligonucleotide
tgcacctacccttttc

SEQ ID NO: 1315 - DNA, 16 bp, Artificial sequence
  Synthetic oligonucleotide
acacataaaggacccc

SEQ ID NO: 1316 - DNA, 16 bp, Artificial sequence
  Synthetic oligonucleotide
gcattaccaggcacct

SEQ ID NO: 1317 - DNA, 16 bp, Artificial sequence
  Synthetic oligonucleotide
gacatcacaggtgttg

SEQ ID NO: 1318 - DNA, 16 bp, Artificial sequence
  Synthetic oligonucleotide
gtgtctcggccagggc

SEQ ID NO: 1319 - DNA, 16 bp, Artificial sequence
  Synthetic oligonucleotide
gtcctgatccgcagca

SEQ ID NO: 1320 - DNA, 16 bp, Artificial sequence
  Synthetic oligonucleotide
ctccagccgcgctctg

SEQ ID NO: 1321 - DNA, 16 bp, Artificial sequence
  Synthetic oligonucleotide
aggcagcgggtcgccc

SEQ ID NO: 1322 - DNA, 16 bp, Artificial sequence
  Synthetic oligonucleotide
gcgggataccggagag

SEQ ID NO: 1323 - DNA, 16 bp, Artificial sequence
  Synthetic oligonucleotide
tccgcacaagatctga

SEQ ID NO: 1324 - DNA, 16 bp, Artificial sequence
  Synthetic oligonucleotide
agatgccaatgttccg

SEQ ID NO: 1325 - DNA, 16 bp, Artificial sequence
  Synthetic oligonucleotide
ctgtcggaggaacttg

SEQ ID NO: 1326 - DNA, 16 bp, Artificial sequence
  Synthetic oligonucleotide
acattggccgggaggc

SEQ ID NO: 1327 - DNA, 16 bp, Artificial sequence
  Synthetic oligonucleotide
atgcctattttgccgg

SEQ ID NO: 1328 - DNA, 16 bp, Artificial sequence
  Synthetic oligonucleotide
atcagacactctggta

SEQ ID NO: 1329 - DNA, 16 bp, Artificial sequence
  Synthetic oligonucleotide
actttagggcagatgt

SEQ ID NO: 1330 - DNA, 16 bp, Artificial sequence
  Synthetic oligonucleotide
gtagaggttccctgtg

SEQ ID NO: 1331 - DNA, 16 bp, Artificial sequence
  Synthetic oligonucleotide
agcaccttgagatccg

SEQ ID NO: 1332 - DNA, 16 bp, Artificial sequence
  Synthetic oligonucleotide
gttgcagatgcccttc

SEQ ID NO: 1333 - DNA, 16 bp, Artificial sequence
  Synthetic oligonucleotide
ggcgacctcaggatcc

SEQ ID NO: 1334 - DNA, 16 bp, Artificial sequence
  Synthetic oligonucleotide
aggcagccgactccgg

SEQ ID NO: 1335 - DNA, 16 bp, Artificial sequence
  Synthetic oligonucleotide
gtgtccaggatgctct

SEQ ID NO: 1336 - DNA, 16 bp, Artificial sequence
  Synthetic oligonucleotide
tcactcagtgctgtag

SEQ ID NO: 1337 - DNA, 16 bp, Artificial sequence
  Synthetic oligonucleotide
ataagacattatccta

SEQ ID NO: 1338 - DNA, 16 bp, Artificial sequence
  Synthetic oligonucleotide
acaatcgcaatggcag

SEQ ID NO: 1339 - DNA, 16 bp, Artificial sequence
  Synthetic oligonucleotide
gtgaacacctgtgagg

SEQ ID NO: 1340 - DNA, 16 bp, Artificial sequence
  Synthetic oligonucleotide
ttggctgctcactggc

SEQ ID NO: 1341 - DNA, 16 bp, Artificial sequence
  Synthetic oligonucleotide
gggccagtcctgctca

SEQ ID NO: 1342 - DNA, 16 bp, Artificial sequence
  Synthetic oligonucleotide
gcccaagaagaagttc

SEQ ID NO: 1343 - DNA, 16 bp, Artificial sequence
  Synthetic oligonucleotide
ctagtgaaaaactggg

SEQ ID NO: 1344 - DNA, 16 bp, Artificial sequence
  Synthetic oligonucleotide
gctagactcgcctcct

SEQ ID NO: 1345 - DNA, 16 bp, Artificial sequence
  Synthetic oligonucleotide
tgcggaggtagctgca

SEQ ID NO: 1346 - DNA, 16 bp, Artificial sequence
  Synthetic oligonucleotide
cctagcttttcataaa

SEQ ID NO: 1347 - DNA, 16 bp, Artificial sequence
  Synthetic oligonucleotide
gaccgctgcacaggcg

SEQ ID NO: 1348 - DNA, 16 bp, Artificial sequence
  Synthetic oligonucleotide
atgctgatgtattaga

SEQ ID NO: 1349 - DNA, 16 bp, Artificial sequence
  Synthetic oligonucleotide
tcccaaccagctgaat

SEQ ID NO: 1350 - DNA, 16 bp, Artificial sequence
  Synthetic oligonucleotide
gaaacagtcagtaagg

SEQ ID NO: 1351 - DNA, 16 bp, Artificial sequence
  Synthetic oligonucleotide
agtctgaccattaata

SEQ ID NO: 1352 - DNA, 16 bp, Artificial sequence
  Synthetic oligonucleotide
acctgtcattctaaga

SEQ ID NO: 1353 - DNA, 16 bp, Artificial sequence
  Synthetic oligonucleotide
cagcctaccccccatc

SEQ ID NO: 1354 - DNA, 16 bp, Artificial sequence
  Synthetic oligonucleotide
ctccaccccacaagat

SEQ ID NO: 1355 - DNA, 16 bp, Artificial sequence
  Synthetic oligonucleotide
ctttgcagaccacctg

SEQ ID NO: 1356 - DNA, 16 bp, Artificial sequence
  Synthetic oligonucleotide
tacccccgccatggag

SEQ ID NO: 1357 - DNA, 16 bp, Artificial sequence
  Synthetic oligonucleotide
caacaggtaacaacgc

SEQ ID NO: 1358 - DNA, 16 bp, Artificial sequence
  Synthetic oligonucleotide
gtcagactttcactca

SEQ ID NO: 1359 - DNA, 16 bp, Artificial sequence
  Synthetic oligonucleotide
gtgcttggctcctgcc

SEQ ID NO: 1360 - DNA, 16 bp, Artificial sequence
  Synthetic oligonucleotide
caagttgtgtgctcca

SEQ ID NO: 1361 - DNA, 16 bp, Artificial sequence
  Synthetic oligonucleotide
catcgccacacatggg

SEQ ID NO: 1362 - DNA, 16 bp, Artificial sequence
  Synthetic oligonucleotide
aggaagggtcccaaac

SEQ ID NO: 1363 - DNA, 16 bp, Artificial sequence
  Synthetic oligonucleotide
tgatcatgtggcggtc

SEQ ID NO: 1364 - DNA, 16 bp, Artificial sequence
  Synthetic oligonucleotide
tgctatcaggtgcagg

SEQ ID NO: 1365 - DNA, 16 bp, Artificial sequence
  Synthetic oligonucleotide
tatattgggctcaatg

SEQ ID NO: 1366 - DNA, 16 bp, Artificial sequence
  Synthetic oligonucleotide
gtttacaaacatggac

SEQ ID NO: 1367 - DNA, 16 bp, Artificial sequence
  Synthetic oligonucleotide
tcattagcatcaccgg

SEQ ID NO: 1368 - DNA, 16 bp, Artificial sequence
  Synthetic oligonucleotide
gggaactaacatacac

SEQ ID NO: 1369 - DNA, 16 bp, Artificial sequence
  Synthetic oligonucleotide
gggtatatgttcccag

SEQ ID NO: 1370 - DNA, 16 bp, Artificial sequence
  Synthetic oligonucleotide
tgcatagccttctttc

SEQ ID NO: 1371 - DNA, 16 bp, Artificial sequence
  Synthetic oligonucleotide
catcattggaagaccg

SEQ ID NO: 1372 - DNA, 16 bp, Artificial sequence
  Synthetic oligonucleotide
tctttaacttcggccc

SEQ ID NO: 1373 - DNA, 16 bp, Artificial sequence
  Synthetic oligonucleotide
tcttaatgtggattca

SEQ ID NO: 1374 - DNA, 16 bp, Artificial sequence
  Synthetic oligonucleotide
tcagacaaccacagct

SEQ ID NO: 1375 - DNA, 16 bp, Artificial sequence
  Synthetic oligonucleotide
taaagcaggacacacg

SEQ ID NO: 1376 - DNA, 16 bp, Artificial sequence
  Synthetic oligonucleotide
agacatgttggtgtct

SEQ ID NO: 1377 - DNA, 16 bp, Artificial sequence
  Synthetic oligonucleotide
ccccagtcttttattc

SEQ ID NO: 1378 - DNA, 16 bp, Artificial sequence
  Synthetic oligonucleotide
ggaagacacggagcca

SEQ ID NO: 1379 - DNA, 16 bp, Artificial sequence
  Synthetic oligonucleotide
cctaactgctggctct

SEQ ID NO: 1380 - DNA, 16 bp, Artificial sequence
  Synthetic oligonucleotide
taattttacgatcatc

SEQ ID NO: 1381 - DNA, 16 bp, Artificial sequence
  Synthetic oligonucleotide
ctctttgtagcagaca

SEQ ID NO: 1382 - DNA, 16 bp, Artificial sequence
  Synthetic oligonucleotide
caatatactgagagga

SEQ ID NO: 1383 - DNA, 16 bp, Artificial sequence
  Synthetic oligonucleotide
gtagacatccttcccg

SEQ ID NO: 1384 - DNA, 16 bp, Artificial sequence
  Synthetic oligonucleotide
gacacataaaggaccc

SEQ ID NO: 1385 - DNA, 16 bp, Artificial sequence
  Synthetic oligonucleotide
tgcattaccaggcacc

SEQ ID NO: 1386 - DNA, 16 bp, Artificial sequence
  Synthetic oligonucleotide
ggacatcacaggtgtt

SEQ ID NO: 1387 - DNA, 16 bp, Artificial sequence
  Synthetic oligonucleotide
agtgtctcggccaggg

SEQ ID NO: 1388 - DNA, 16 bp, Artificial sequence
  Synthetic oligonucleotide
ggtcctgatccgcagc

SEQ ID NO: 1389 - DNA, 16 bp, Artificial sequence
  Synthetic oligonucleotide
gctccagccgcgctct

SEQ ID NO: 1390 - DNA, 16 bp, Artificial sequence
  Synthetic oligonucleotide
caggcagcgggtcgcc

SEQ ID NO: 1391 - DNA, 16 bp, Artificial sequence
  Synthetic oligonucleotide
agcgggataccggaga

SEQ ID NO: 1392 - DNA, 16 bp, Artificial sequence
  Synthetic oligonucleotide
ttccgcacaagatctg

SEQ ID NO: 1393 - DNA, 16 bp, Artificial sequence
  Synthetic oligonucleotide
aagatgccaatgttcc

SEQ ID NO: 1394 - DNA, 16 bp, Artificial sequence
  Synthetic oligonucleotide
ccctgtcggaggaact

SEQ ID NO: 1395 - DNA, 16 bp, Artificial sequence
  Synthetic oligonucleotide
ggacattggccgggag

SEQ ID NO: 1396 - DNA, 16 bp, Artificial sequence
  Synthetic oligonucleotide
gatgcctattttgccg

SEQ ID NO: 1397 - DNA, 16 bp, Artificial sequence
  Synthetic oligonucleotide
ccatcagacactctgg

SEQ ID NO: 1398 - DNA, 16 bp, Artificial sequence
  Synthetic oligonucleotide
caggaacataccaagg

SEQ ID NO: 1399 - DNA, 16 bp, Artificial sequence
  Synthetic oligonucleotide
gttgtcactcactcct

SEQ ID NO: 1400 - DNA, 16 bp, Artificial sequence
  Synthetic oligonucleotide
gactttagggcagatg

SEQ ID NO: 1401 - DNA, 16 bp, Artificial sequence
  Synthetic oligonucleotide
ggtagaggttccctgt

SEQ ID NO: 1402 - DNA, 16 bp, Artificial sequence
  Synthetic oligonucleotide
cagcaccttgagatcc

SEQ ID NO: 1403 - DNA, 16 bp, Artificial sequence
  Synthetic oligonucleotide
ctgttgcagatgccct

SEQ ID NO: 1404 - DNA, 16 bp, Artificial sequence
  Synthetic oligonucleotide
tggcgacctcaggatc

SEQ ID NO: 1405 - DNA, 16 bp, Artificial sequence
  Synthetic oligonucleotide
aaggcagccgactccg

SEQ ID NO: 1406 - DNA, 16 bp, Artificial sequence
  Synthetic oligonucleotide
ggtgtccaggatgctc

SEQ ID NO: 1407 - DNA, 16 bp, Artificial sequence
  Synthetic oligonucleotide
ttcactcagtgctgta

SEQ ID NO: 1408 - DNA, 16 bp, Artificial sequence
  Synthetic oligonucleotide
acataagacattatcc

SEQ ID NO: 1409 - DNA, 16 bp, Artificial sequence
  Synthetic oligonucleotide
ctggacaatcgcaatg

SEQ ID NO: 1410 - DNA, 16 bp, Artificial sequence
  Synthetic oligonucleotide
gagtgaacacctgtga

SEQ ID NO: 1411 - DNA, 16 bp, Artificial sequence
  Synthetic oligonucleotide
gttggctgctcactgg

SEQ ID NO: 1412 - DNA, 16 bp, Artificial sequence
  Synthetic oligonucleotide
agggccagtcctgctc

SEQ ID NO: 1413 - DNA, 16 bp, Artificial sequence
  Synthetic oligonucleotide
attgcccaagaagaag

SEQ ID NO: 1414 - DNA, 16 bp, Artificial sequence
  Synthetic oligonucleotide
tctagtgaaaaactgg

SEQ ID NO: 1415 - DNA, 16 bp, Artificial sequence
  Synthetic oligonucleotide
tgctagactcgcctcc

SEQ ID NO: 1416 - DNA, 16 bp, Artificial sequence
  Synthetic oligonucleotide
atgcggaggtagctgc

SEQ ID NO: 1417 - DNA, 16 bp, Artificial sequence
  Synthetic oligonucleotide
ggttgcttcctagctt

SEQ ID NO: 1418 - DNA, 16 bp, Artificial sequence
  Synthetic oligonucleotide
ggaccgctgcacaggc

SEQ ID NO: 1419 - DNA, 16 bp, Artificial sequence
  Synthetic oligonucleotide
catgctgatgtattag

SEQ ID NO: 1420 - DNA, 16 bp, Artificial sequence
  Synthetic oligonucleotide
ttcccaaccagctgaa

SEQ ID NO: 1421 - DNA, 16 bp, Artificial sequence
  Synthetic oligonucleotide
cgaaacagtcagtaag

SEQ ID NO: 1422 - DNA, 16 bp, Artificial sequence
  Synthetic oligonucleotide
aacagtctgaccatta

SEQ ID NO: 1423 - DNA, 16 bp, Artificial sequence
  Synthetic oligonucleotide
cacctgtcattctaag

SEQ ID NO: 1424 - DNA, 16 bp, Artificial sequence
  Synthetic oligonucleotide
ccagcctaccccccat

SEQ ID NO: 1425 - DNA, 16 bp, Artificial sequence
  Synthetic oligonucleotide
gtgggatcatgctatt

SEQ ID NO: 1426 - DNA, 16 bp, Artificial sequence
  Synthetic oligonucleotide
tctttgcagaccacct

SEQ ID NO: 1427 - DNA, 16 bp, Artificial sequence
  Synthetic oligonucleotide
ttacccccgccatgga

SEQ ID NO: 1428 - DNA, 16 bp, Artificial sequence
  Synthetic oligonucleotide
tcaacaggtaacaacg

SEQ ID NO: 1429 - DNA, 16 bp, Artificial sequence
  Synthetic oligonucleotide
gcacactagattattt

SEQ ID NO: 1430 - DNA, 16 bp, Artificial sequence
  Synthetic oligonucleotide
cggaagctcctgctgt

SEQ ID NO: 1431 - DNA, 16 bp, Artificial sequence
  Synthetic oligonucleotide
tcaagttgtgtgctcc

SEQ ID NO: 1432 - DNA, 16 bp, Artificial sequence
  Synthetic oligonucleotide
tcatcgccacacatgg

SEQ ID NO: 1433 - DNA, 16 bp, Artificial sequence
  Synthetic oligonucleotide
tcatttccaggagtac

SEQ ID NO: 1434 - DNA, 16 bp, Artificial sequence
  Synthetic oligonucleotide
caaatgatcatgtggc

SEQ ID NO: 1435 - DNA, 16 bp, Artificial sequence
  Synthetic oligonucleotide
taatgctatcaggtgc

SEQ ID NO: 1436 - DNA, 16 bp, Artificial sequence
  Synthetic oligonucleotide
gatatattgggctcaa

SEQ ID NO: 1437 - DNA, 16 bp, Artificial sequence
  Synthetic oligonucleotide
gggtttacaaacatgg

SEQ ID NO: 1438 - DNA, 16 bp, Artificial sequence
  Synthetic oligonucleotide
ttcattagcatcaccg

SEQ ID NO: 1439 - DNA, 16 bp, Artificial sequence
  Synthetic oligonucleotide
gttaatccatgggtca

SEQ ID NO: 1440 - DNA, 16 bp, Artificial sequence
  Synthetic oligonucleotide
agcctaaacttcctcc

SEQ ID NO: 1441 - DNA, 16 bp, Artificial sequence
  Synthetic oligonucleotide
agaagagccgccctgc

SEQ ID NO: 1442 - DNA, 16 bp, Artificial sequence
  Synthetic oligonucleotide
gcaagactagcaagtg

SEQ ID NO: 1443 - DNA, 16 bp, Artificial sequence
  Synthetic oligonucleotide
agcatgcggtatgtac

SEQ ID NO: 1444 - DNA, 16 bp, Artificial sequence
  Synthetic oligonucleotide
cacacaatttctaggg

SEQ ID NO: 1445 - DNA, 16 bp, Artificial sequence
  Synthetic oligonucleotide
ttgacaattagaacca

SEQ ID NO: 1446 - DNA, 16 bp, Artificial sequence
  Synthetic oligonucleotide
acaaatccttaccgag

SEQ ID NO: 1447 - DNA, 16 bp, Artificial sequence
  Synthetic oligonucleotide
gttttaggtctgggta

SEQ ID NO: 1448 - DNA, 16 bp, Artificial sequence
  Synthetic oligonucleotide
ttgcatagccttcttt

SEQ ID NO: 1449 - DNA, 16 bp, Artificial sequence
  Synthetic oligonucleotide
catacatacccttctc

SEQ ID NO: 1450 - DNA, 16 bp, Artificial sequence
  Synthetic oligonucleotide
cgcagaaactctttaa

SEQ ID NO: 1451 - DNA, 16 bp, Artificial sequence
  Synthetic oligonucleotide
gtcttaatgtggattc

SEQ ID NO: 1452 - DNA, 16 bp, Artificial sequence
  Synthetic oligonucleotide
agcattggcacactgg

SEQ ID NO: 1453 - DNA, 16 bp, Artificial sequence
  Synthetic oligonucleotide
ggctttaaagcaggac

SEQ ID NO: 1454 - DNA, 16 bp, Artificial sequence
  Synthetic oligonucleotide
gcagacatgttggtgt

SEQ ID NO: 1455 - DNA, 16 bp, Artificial sequence
  Synthetic oligonucleotide
tacaagctggtccttg

SEQ ID NO: 1456 - DNA, 16 bp, Artificial sequence
  Synthetic oligonucleotide
gacaatccaggtccca

SEQ ID NO: 1457 - DNA, 16 bp, Artificial sequence
  Synthetic oligonucleotide
gaggaagcccaatcaa

SEQ ID NO: 1458 - DNA, 16 bp, Artificial sequence
  Synthetic oligonucleotide
ctaattttacgatcat

SEQ ID NO: 1459 - DNA, 16 bp, Artificial sequence
  Synthetic oligonucleotide
ttaaactgccaagtcc

SEQ ID NO: 1460 - DNA, 16 bp, Artificial sequence
  Synthetic oligonucleotide
ccaatatactgagagg

SEQ ID NO: 1461 - DNA, 16 bp, Artificial sequence
  Synthetic oligonucleotide
ggtagcaccgccaagt

SEQ ID NO: 1462 - DNA, 16 bp, Artificial sequence
  Synthetic oligonucleotide
caccatggagaggtct

SEQ ID NO: 1463 - DNA, 16 bp, Artificial sequence
  Synthetic oligonucleotide
ttgcattaccaggcac

SEQ ID NO: 1464 - DNA, 16 bp, Artificial sequence
  Synthetic oligonucleotide
gctacctggacacctc

SEQ ID NO: 1465 - DNA, 16 bp, Artificial sequence
  Synthetic oligonucleotide
atctttgcagaccacc

SEQ ID NO: 1466 - DNA, 16 bp, Artificial sequence
  Synthetic oligonucleotide
cagtgtctcggccagg

SEQ ID NO: 1467 - DNA, 16 bp, Artificial sequence
  Synthetic oligonucleotide
gggtcctgatccgcag

SEQ ID NO: 1468 - DNA, 16 bp, Artificial sequence
  Synthetic oligonucleotide
agctccagccgcgctc

SEQ ID NO: 1469 - DNA, 16 bp, Artificial sequence
  Synthetic oligonucleotide
tcaggcagcgggtcgc

SEQ ID NO: 1470 - DNA, 16 bp, Artificial sequence
  Synthetic oligonucleotide
cagcgggataccggag

SEQ ID NO: 1471 - DNA, 16 bp, Artificial sequence
  Synthetic oligonucleotide
cttccgcacaagatct

SEQ ID NO: 1472 - DNA, 16 bp, Artificial sequence
  Synthetic oligonucleotide
ggaagatgccaatgtt

SEQ ID NO: 1473 - DNA, 16 bp, Artificial sequence
  Synthetic oligonucleotide
accctgtcggaggaac

SEQ ID NO: 1474 - DNA, 16 bp, Artificial sequence
  Synthetic oligonucleotide
ggtggacattggccgg

SEQ ID NO: 1475 - DNA, 16 bp, Artificial sequence
  Synthetic oligonucleotide
agatgcctattttgcc

SEQ ID NO: 1476 - DNA, 16 bp, Artificial sequence
  Synthetic oligonucleotide
cgaaagtcagacacca

SEQ ID NO: 1477 - DNA, 16 bp, Artificial sequence
  Synthetic oligonucleotide
tgactttagggcagat

SEQ ID NO: 1478 - DNA, 16 bp, Artificial sequence
  Synthetic oligonucleotide
aaggtagaggttccct

SEQ ID NO: 1479 - DNA, 16 bp, Artificial sequence
  Synthetic oligonucleotide
aaggcatatctctccc

SEQ ID NO: 1480 - DNA, 16 bp, Artificial sequence
  Synthetic oligonucleotide
cctgttgcagatgccc

SEQ ID NO: 1481 - DNA, 16 bp, Artificial sequence
  Synthetic oligonucleotide
atggcgacctcaggat

SEQ ID NO: 1482 - DNA, 16 bp, Artificial sequence
  Synthetic oligonucleotide
caaggcagccgactcc

SEQ ID NO: 1483 - DNA, 16 bp, Artificial sequence
  Synthetic oligonucleotide
cgagagggtgtccagg

SEQ ID NO: 1484 - DNA, 16 bp, Artificial sequence
  Synthetic oligonucleotide
cttcactcagtgctgt

SEQ ID NO: 1485 - DNA, 16 bp, Artificial sequence
  Synthetic oligonucleotide
cagcattacataagac

SEQ ID NO: 1486 - DNA, 16 bp, Artificial sequence
  Synthetic oligonucleotide
ctctggacaatcgcaa

SEQ ID NO: 1487 - DNA, 16 bp, Artificial sequence
  Synthetic oligonucleotide
cgagtgaacacctgtg

SEQ ID NO: 1488 - DNA, 16 bp, Artificial sequence
  Synthetic oligonucleotide
tgttggctgctcactg

SEQ ID NO: 1489 - DNA, 16 bp, Artificial sequence
  Synthetic oligonucleotide
ctggacagcccttggg

SEQ ID NO: 1490 - DNA, 16 bp, Artificial sequence
  Synthetic oligonucleotide
tattgcccaagaagaa

SEQ ID NO: 1491 - DNA, 16 bp, Artificial sequence
  Synthetic oligonucleotide
actcttctctagtgaa

SEQ ID NO: 1492 - DNA, 16 bp, Artificial sequence
  Synthetic oligonucleotide
ctgctagactcgcctc

SEQ ID NO: 1493 - DNA, 16 bp, Artificial sequence
  Synthetic oligonucleotide
aatgcggaggtagctg

SEQ ID NO: 1494 - DNA, 16 bp, Artificial sequence
  Synthetic oligonucleotide
aggttgcttcctagct

SEQ ID NO: 1495 - DNA, 16 bp, Artificial sequence
  Synthetic oligonucleotide
tggaccgctgcacagg

SEQ ID NO: 1496 - DNA, 16 bp, Artificial sequence
  Synthetic oligonucleotide
gcatgctgatgtatta

SEQ ID NO: 1497 - DNA, 16 bp, Artificial sequence
  Synthetic oligonucleotide
tcatttcccaaccagc

SEQ ID NO: 1498 - DNA, 16 bp, Artificial sequence
  Synthetic oligonucleotide
acgaaacagtcagtaa

SEQ ID NO: 1499 - DNA, 16 bp, Artificial sequence
  Synthetic oligonucleotide
ggaacagtctgaccat

SEQ ID NO: 1500 - DNA, 16 bp, Artificial sequence
  Synthetic oligonucleotide
aacacctgtcattcta

SEQ ID NO: 1501 - DNA, 16 bp, Artificial sequence
  Synthetic oligonucleotide
gccagcctacccccca

SEQ ID NO: 1502 - DNA, 16 bp, Artificial sequence
  Synthetic oligonucleotide
agtgggatcatgctat

SEQ ID NO: 1503 - DNA, 16 bp, Artificial sequence
  Synthetic oligonucleotide
gttacccccgccatgg

SEQ ID NO: 1504 - DNA, 16 bp, Artificial sequence
  Synthetic oligonucleotide
ttcaacaggtaacaac

SEQ ID NO: 1505 - DNA, 16 bp, Artificial sequence
  Synthetic oligonucleotide
tgcacactagattatt

SEQ ID NO: 1506 - DNA, 16 bp, Artificial sequence
  Synthetic oligonucleotide
gcggaagctcctgctg

SEQ ID NO: 1507 - DNA, 16 bp, Artificial sequence
  Synthetic oligonucleotide
gttcaagttgtgtgct

SEQ ID NO: 1508 - DNA, 16 bp, Artificial sequence
  Synthetic oligonucleotide
ctcatcgccacacatg

SEQ ID NO: 1509 - DNA, 16 bp, Artificial sequence
  Synthetic oligonucleotide
cggaatctcatttcca

SEQ ID NO: 1510 - DNA, 16 bp, Artificial sequence
  Synthetic oligonucleotide
gcaaatgatcatgtgg

SEQ ID NO: 1511 - DNA, 16 bp, Artificial sequence
  Synthetic oligonucleotide
cttaatgctatcaggt

SEQ ID NO: 1512 - DNA, 16 bp, Artificial sequence
  Synthetic oligonucleotide
ggatatattgggctca

SEQ ID NO: 1513 - DNA, 16 bp, Artificial sequence
  Synthetic oligonucleotide
agggtttacaaacatg

SEQ ID NO: 1514 - DNA, 16 bp, Artificial sequence
  Synthetic oligonucleotide
attcattagcatcacc

SEQ ID NO: 1515 - DNA, 16 bp, Artificial sequence
  Synthetic oligonucleotide
ggttaatccatgggtc

SEQ ID NO: 1516 - DNA, 16 bp, Artificial sequence
  Synthetic oligonucleotide
agttttaggtctgggt

SEQ ID NO: 1517 - DNA, 16 bp, Artificial sequence
  Synthetic oligonucleotide
cattgcatagccttct

SEQ ID NO: 1518 - DNA, 16 bp, Artificial sequence
  Synthetic oligonucleotide
ccatacatacccttct

SEQ ID NO: 1519 - DNA, 16 bp, Artificial sequence
  Synthetic oligonucleotide
acccacacctgactgg

SEQ ID NO: 1520 - DNA, 16 bp, Artificial sequence
  Synthetic oligonucleotide
cgctcctacttatccc

SEQ ID NO: 1521 - DNA, 16 bp, Artificial sequence
  Synthetic oligonucleotide
tcttacagcattggca

SEQ ID NO: 1522 - DNA, 16 bp, Artificial sequence
  Synthetic oligonucleotide
catctaccaaactgca

SEQ ID NO: 1523 - DNA, 16 bp, Artificial sequence
  Synthetic oligonucleotide
aacaaacatcgatttt

SEQ ID NO: 1524 - DNA, 16 bp, Artificial sequence
  Synthetic oligonucleotide
agctttacaagctggt

SEQ ID NO: 1525 - DNA, 16 bp, Artificial sequence
  Synthetic oligonucleotide
acgacaatccaggtcc

SEQ ID NO: 1526 - DNA, 16 bp, Artificial sequence
  Synthetic oligonucleotide
tctaattttacgatca

SEQ ID NO: 1527 - DNA, 16 bp, Artificial sequence
  Synthetic oligonucleotide
attaaactgccaagtc

SEQ ID NO: 1528 - DNA, 16 bp, Artificial sequence
  Synthetic oligonucleotide
accaatatactgagag

SEQ ID NO: 1529 - DNA, 16 bp, Artificial sequence
  Synthetic oligonucleotide
agcggtagcaccgcca

SEQ ID NO: 1530 - DNA, 16 bp, Artificial sequence
  Synthetic oligonucleotide
tcacatgtgagcccag

SEQ ID NO: 1531 - DNA, 16 bp, Artificial sequence
  Synthetic oligonucleotide
gtacaacagagggtgg

SEQ ID NO: 1532 - DNA, 16 bp, Artificial sequence
  Synthetic oligonucleotide
ggctgatgtcaccacc

SEQ ID NO: 1533 - DNA, 16 bp, Artificial sequence
  Synthetic oligonucleotide
tcagtgtctcggccag

SEQ ID NO: 1534 - DNA, 16 bp, Artificial sequence
  Synthetic oligonucleotide
tcggctcgggtcctga

SEQ ID NO: 1535 - DNA, 16 bp, Artificial sequence
  Synthetic oligonucleotide
caagctccagccgcgc

SEQ ID NO: 1536 - DNA, 16 bp, Artificial sequence
  Synthetic oligonucleotide
ctcaggcagcgggtcg

SEQ ID NO: 1537 - DNA, 16 bp, Artificial sequence
  Synthetic oligonucleotide
ccagcgggataccgga

SEQ ID NO: 1538 - DNA, 16 bp, Artificial sequence
  Synthetic oligonucleotide
ccttccgcacaagatc

SEQ ID NO: 1539 - DNA, 16 bp, Artificial sequence
  Synthetic oligonucleotide
gatggaagatgccaat

SEQ ID NO: 1540 - DNA, 16 bp, Artificial sequence
  Synthetic oligonucleotide
gaccctgtcggaggaa

SEQ ID NO: 1541 - DNA, 16 bp, Artificial sequence
  Synthetic oligonucleotide
ctggtggacattggcc

SEQ ID NO: 1542 - DNA, 16 bp, Artificial sequence
  Synthetic oligonucleotide
gagatgcctattttgc

SEQ ID NO: 1543 - DNA, 16 bp, Artificial sequence
  Synthetic oligonucleotide
ccgaaagtcagacacc

SEQ ID NO: 1544 - DNA, 16 bp, Artificial sequence
  Synthetic oligonucleotide
gcatcaatgaagggta

SEQ ID NO: 1545 - DNA, 16 bp, Artificial sequence
  Synthetic oligonucleotide
ttgactttagggcaga

SEQ ID NO: 1546 - DNA, 16 bp, Artificial sequence
  Synthetic oligonucleotide
gaaggtagaggttccc

SEQ ID NO: 1547 - DNA, 16 bp, Artificial sequence
  Synthetic oligonucleotide
gaaggcatatctctcc

SEQ ID NO: 1548 - DNA, 16 bp, Artificial sequence
  Synthetic oligonucleotide
gcctgttgcagatgcc

SEQ ID NO: 1549 - DNA, 16 bp, Artificial sequence
  Synthetic oligonucleotide
catggcgacctcagga

SEQ ID NO: 1550 - DNA, 16 bp, Artificial sequence
  Synthetic oligonucleotide
ccaaggcagccgactc

SEQ ID NO: 1551 - DNA, 16 bp, Artificial sequence
  Synthetic oligonucleotide
gcgagagggtgtccag

SEQ ID NO: 1552 - DNA, 16 bp, Artificial sequence
  Synthetic oligonucleotide
tcttcactcagtgctg

SEQ ID NO: 1553 - DNA, 16 bp, Artificial sequence
  Synthetic oligonucleotide
gcagcattacataaga

SEQ ID NO: 1554 - DNA, 16 bp, Artificial sequence
  Synthetic oligonucleotide
agtctctggacaatcg

SEQ ID NO: 1555 - DNA, 16 bp, Artificial sequence
  Synthetic oligonucleotide
tcgagtgaacacctgt

SEQ ID NO: 1556 - DNA, 16 bp, Artificial sequence
  Synthetic oligonucleotide
ctgttggctgctcact

SEQ ID NO: 1557 - DNA, 16 bp, Artificial sequence
  Synthetic oligonucleotide
gctggacagcccttgg

SEQ ID NO: 1558 - DNA, 16 bp, Artificial sequence
  Synthetic oligonucleotide
ttattgcccaagaaga

SEQ ID NO: 1559 - DNA, 16 bp, Artificial sequence
  Synthetic oligonucleotide
gactcttctctagtga

SEQ ID NO: 1560 - DNA, 16 bp, Artificial sequence
  Synthetic oligonucleotide
tctgctagactcgcct

SEQ ID NO: 1561 - DNA, 16 bp, Artificial sequence
  Synthetic oligonucleotide
caatgcggaggtagct

SEQ ID NO: 1562 - DNA, 16 bp, Artificial sequence
  Synthetic oligonucleotide
aaggttgcttcctagc

SEQ ID NO: 1563 - DNA, 16 bp, Artificial sequence
  Synthetic oligonucleotide
ctggaccgctgcacag

SEQ ID NO: 1564 - DNA, 16 bp, Artificial sequence
  Synthetic oligonucleotide
cgcatgctgatgtatt

SEQ ID NO: 1565 - DNA, 16 bp, Artificial sequence
  Synthetic oligonucleotide
gtgtcatttcccaacc

SEQ ID NO: 1566 - DNA, 16 bp, Artificial sequence
  Synthetic oligonucleotide
cacgaaacagtcagta

SEQ ID NO: 1567 - DNA, 16 bp, Artificial sequence
  Synthetic oligonucleotide
gctggaacagtctgac

SEQ ID NO: 1568 - DNA, 16 bp, Artificial sequence
  Synthetic oligonucleotide
catccaaacacctgtc

SEQ ID NO: 1569 - DNA, 16 bp, Artificial sequence
  Synthetic oligonucleotide
ggccagcctacccccc

SEQ ID NO: 1570 - DNA, 16 bp, Artificial sequence
  Synthetic oligonucleotide
aagtgggatcatgcta

SEQ ID NO: 1571 - DNA, 16 bp, Artificial sequence
  Synthetic oligonucleotide
catctttgcagaccac

SEQ ID NO: 1572 - DNA, 16 bp, Artificial sequence
  Synthetic oligonucleotide
tgttacccccgccatg

SEQ ID NO: 1573 - DNA, 16 bp, Artificial sequence
  Synthetic oligonucleotide
gattcacataatacaa

SEQ ID NO: 1574 - DNA, 16 bp, Artificial sequence
  Synthetic oligonucleotide
ctgcacactagattat

SEQ ID NO: 1575 - DNA, 16 bp, Artificial sequence
  Synthetic oligonucleotide
ggcggaagctcctgct

SEQ ID NO: 1576 - DNA, 16 bp, Artificial sequence
  Synthetic oligonucleotide
ggttcaagttgtgtgc

SEQ ID NO: 1577 - DNA, 16 bp, Artificial sequence
  Synthetic oligonucleotide
tctcatcgccacacat

SEQ ID NO: 1578 - DNA, 16 bp, Artificial sequence
  Synthetic oligonucleotide
tacggaatctcatttc

SEQ ID NO: 1579 - DNA, 16 bp, Artificial sequence
  Synthetic oligonucleotide
ggccaccttgggatac

SEQ ID NO: 1580 - DNA, 16 bp, Artificial sequence
  Synthetic oligonucleotide
gccttaatgctatcag

SEQ ID NO: 1581 - DNA, 16 bp, Artificial sequence
  Synthetic oligonucleotide
tggatatattgggctc

SEQ ID NO: 1582 - DNA, 16 bp, Artificial sequence
  Synthetic oligonucleotide
acattcagggtttaca

SEQ ID NO: 1583 - DNA, 16 bp, Artificial sequence
  Synthetic oligonucleotide
gtattcattagcatca

SEQ ID NO: 1584 - DNA, 16 bp, Artificial sequence
  Synthetic oligonucleotide
aggttaatccatgggt

SEQ ID NO: 1585 - DNA, 16 bp, Artificial sequence
  Synthetic oligonucleotide
gagttttaggtctggg

SEQ ID NO: 1586 - DNA, 16 bp, Artificial sequence
  Synthetic oligonucleotide
cttataaagcacacgg

SEQ ID NO: 1587 - DNA, 16 bp, Artificial sequence
  Synthetic oligonucleotide
gggcatggctgatcct

SEQ ID NO: 1588 - DNA, 16 bp, Artificial sequence
  Synthetic oligonucleotide
caaacttgtctagtgg

SEQ ID NO: 1589 - DNA, 16 bp, Artificial sequence
  Synthetic oligonucleotide
tcgcatccatgggtcc

SEQ ID NO: 1590 - DNA, 16 bp, Artificial sequence
  Synthetic oligonucleotide
gctcttacagcattgg

SEQ ID NO: 1591 - DNA, 16 bp, Artificial sequence
  Synthetic oligonucleotide
ccatctaccaaactgc

SEQ ID NO: 1592 - DNA, 16 bp, Artificial sequence
  Synthetic oligonucleotide
gacttagtccgtgttc

SEQ ID NO: 1593 - DNA, 16 bp, Artificial sequence
  Synthetic oligonucleotide
cagcatctatgttctc

SEQ ID NO: 1594 - DNA, 16 bp, Artificial sequence
  Synthetic oligonucleotide
atagactgtgagctgt

SEQ ID NO: 1595 - DNA, 16 bp, Artificial sequence
  Synthetic oligonucleotide
gaccattctgctcccc

SEQ ID NO: 1596 - DNA, 16 bp, Artificial sequence
  Synthetic oligonucleotide
gcccataccttttatc

SEQ ID NO: 1597 - DNA, 16 bp, Artificial sequence
  Synthetic oligonucleotide
gtattaaactgccaag

SEQ ID NO: 1598 - DNA, 16 bp, Artificial sequence
  Synthetic oligonucleotide
ctaaccaatatactga

SEQ ID NO: 1599 - DNA, 16 bp, Artificial sequence
  Synthetic oligonucleotide
ggctggtgatgaaaca

SEQ ID NO: 1600 - DNA, 16 bp, Artificial sequence
  Synthetic oligonucleotide
cctcatggtttgctgt

SEQ ID NO: 1601 - DNA, 16 bp, Artificial sequence
  Synthetic oligonucleotide
cagtacaacagagggt

SEQ ID NO: 1602 - DNA, 16 bp, Artificial sequence
  Synthetic oligonucleotide
cacattgccggccagt

SEQ ID NO: 1603 - DNA, 16 bp, Artificial sequence
  Synthetic oligonucleotide
ctcagtgtctcggcca

SEQ ID NO: 1604 - DNA, 16 bp, Artificial sequence
  Synthetic oligonucleotide
atcggctcgggtcctg

SEQ ID NO: 1605 - DNA, 16 bp, Artificial sequence
  Synthetic oligonucleotide
acaagctccagccgcg

SEQ ID NO: 1606 - DNA, 16 bp, Artificial sequence
  Synthetic oligonucleotide
gctcaggcagcgggtc

SEQ ID NO: 1607 - DNA, 16 bp, Artificial sequence
  Synthetic oligonucleotide
tccagcgggataccgg

SEQ ID NO: 1608 - DNA, 16 bp, Artificial sequence
  Synthetic oligonucleotide
gccttccgcacaagat

SEQ ID NO: 1609 - DNA, 16 bp, Artificial sequence
  Synthetic oligonucleotide
ggatggaagatgccaa

SEQ ID NO: 1610 - DNA, 16 bp, Artificial sequence
  Synthetic oligonucleotide
agaccctgtcggagga

SEQ ID NO: 1611 - DNA, 16 bp, Artificial sequence
  Synthetic oligonucleotide
atgagctggtggacat

SEQ ID NO: 1612 - DNA, 16 bp, Artificial sequence
  Synthetic oligonucleotide
agagatgcctattttg

SEQ ID NO: 1613 - DNA, 16 bp, Artificial sequence
  Synthetic oligonucleotide
accgaaagtcagacac

SEQ ID NO: 1614 - DNA, 16 bp, Artificial sequence
  Synthetic oligonucleotide
ggcatcaatgaagggt

SEQ ID NO: 1615 - DNA, 16 bp, Artificial sequence
  Synthetic oligonucleotide
cttgactttagggcag

SEQ ID NO: 1616 - DNA, 16 bp, Artificial sequence
  Synthetic oligonucleotide
gagaaggtagaggttc

SEQ ID NO: 1617 - DNA, 16 bp, Artificial sequence
  Synthetic oligonucleotide
tcgaaggcatatctct

SEQ ID NO: 1618 - DNA, 16 bp, Artificial sequence
  Synthetic oligonucleotide
ggcctgttgcagatgc

SEQ ID NO: 1619 - DNA, 16 bp, Artificial sequence
  Synthetic oligonucleotide
gcatggcgacctcagg

SEQ ID NO: 1620 - DNA, 16 bp, Artificial sequence
  Synthetic oligonucleotide
gccaaggcagccgact

SEQ ID NO: 1621 - DNA, 16 bp, Artificial sequence
  Synthetic oligonucleotide
ggcgagagggtgtcca

SEQ ID NO: 1622 - DNA, 16 bp, Artificial sequence
  Synthetic oligonucleotide
tgtatccacctttgtc

SEQ ID NO: 1623 - DNA, 16 bp, Artificial sequence
  Synthetic oligonucleotide
ggcagcattacataag

SEQ ID NO: 1624 - DNA, 16 bp, Artificial sequence
  Synthetic oligonucleotide
ccatgtcaccagtctc

SEQ ID NO: 1625 - DNA, 16 bp, Artificial sequence
  Synthetic oligonucleotide
ctcgagtgaacacctg

SEQ ID NO: 1626 - DNA, 16 bp, Artificial sequence
  Synthetic oligonucleotide
cctgttggctgctcac

SEQ ID NO: 1627 - DNA, 16 bp, Artificial sequence
  Synthetic oligonucleotide
tgctggacagcccttg

SEQ ID NO: 1628 - DNA, 16 bp, Artificial sequence
  Synthetic oligonucleotide
tttattgcccaagaag

SEQ ID NO: 1629 - DNA, 16 bp, Artificial sequence
  Synthetic oligonucleotide
agactcttctctagtg

SEQ ID NO: 1630 - DNA, 16 bp, Artificial sequence
  Synthetic oligonucleotide
atctgctagactcgcc

SEQ ID NO: 1631 - DNA, 16 bp, Artificial sequence
  Synthetic oligonucleotide
gcaatgcggaggtagc

SEQ ID NO: 1632 - DNA, 16 bp, Artificial sequence
  Synthetic oligonucleotide
aaaggttgcttcctag

SEQ ID NO: 1633 - DNA, 16 bp, Artificial sequence
  Synthetic oligonucleotide
gctggaccgctgcaca

SEQ ID NO: 1634 - DNA, 16 bp, Artificial sequence
  Synthetic oligonucleotide
acgcatgctgatgtat

SEQ ID NO: 1635 - DNA, 16 bp, Artificial sequence
  Synthetic oligonucleotide
ggtgtcatttcccaac

SEQ ID NO: 1636 - DNA, 16 bp, Artificial sequence
  Synthetic oligonucleotide
ccacgaaacagtcagt

SEQ ID NO: 1637 - DNA, 16 bp, Artificial sequence
  Synthetic oligonucleotide
atgctggaacagtctg

SEQ ID NO: 1638 - DNA, 16 bp, Artificial sequence
  Synthetic oligonucleotide
ccatccaaacacctgt

SEQ ID NO: 1639 - DNA, 16 bp, Artificial sequence
  Synthetic oligonucleotide
gggccagcctaccccc

SEQ ID NO: 1640 - DNA, 16 bp, Artificial sequence
  Synthetic oligonucleotide
gaagtgggatcatgct

SEQ ID NO: 1641 - DNA, 16 bp, Artificial sequence
  Synthetic oligonucleotide
atcatctttgcagacc

SEQ ID NO: 1642 - DNA, 16 bp, Artificial sequence
  Synthetic oligonucleotide
ttgttacccccgccat

SEQ ID NO: 1643 - DNA, 16 bp, Artificial sequence
  Synthetic oligonucleotide
ctgattcacataatac

SEQ ID NO: 1644 - DNA, 16 bp, Artificial sequence
  Synthetic oligonucleotide
cctgcacactagatta

SEQ ID NO: 1645 - DNA, 16 bp, Artificial sequence
  Synthetic oligonucleotide
aggcggaagctcctgc

SEQ ID NO: 1646 - DNA, 16 bp, Artificial sequence
  Synthetic oligonucleotide
aggttcaagttgtgtg

SEQ ID NO: 1647 - DNA, 16 bp, Artificial sequence
  Synthetic oligonucleotide
aatgtacggaatctca

SEQ ID NO: 1648 - DNA, 16 bp, Artificial sequence
  Synthetic oligonucleotide
gtccatgtgggtgtcc

SEQ ID NO: 1649 - DNA, 16 bp, Artificial sequence
  Synthetic oligonucleotide
ggccttaatgctatca

SEQ ID NO: 1650 - DNA, 16 bp, Artificial sequence
  Synthetic oligonucleotide
tagtatgaaatatctc

SEQ ID NO: 1651 - DNA, 16 bp, Artificial sequence
  Synthetic oligonucleotide
attgtaactgccaggc

SEQ ID NO: 1652 - DNA, 16 bp, Artificial sequence
  Synthetic oligonucleotide
ccggtattcattagca

SEQ ID NO: 1653 - DNA, 16 bp, Artificial sequence
  Synthetic oligonucleotide
gagcagggcaacaaac

SEQ ID NO: 1654 - DNA, 16 bp, Artificial sequence
  Synthetic oligonucleotide
atataaccacagcctg

SEQ ID NO: 1655 - DNA, 16 bp, Artificial sequence
  Synthetic oligonucleotide
gcttataaagcacacg

SEQ ID NO: 1656 - DNA, 16 bp, Artificial sequence
  Synthetic oligonucleotide
tagtaaatgcttgtca

SEQ ID NO: 1657 - DNA, 16 bp, Artificial sequence
  Synthetic oligonucleotide
ggcagaaatgtgctct

SEQ ID NO: 1658 - DNA, 16 bp, Artificial sequence
  Synthetic oligonucleotide
cttcatgccatcctgt

SEQ ID NO: 1659 - DNA, 16 bp, Artificial sequence
  Synthetic oligonucleotide
tgctcttacagcattg

SEQ ID NO: 1660 - DNA, 16 bp, Artificial sequence
  Synthetic oligonucleotide
ggtacctgtagcgagc

SEQ ID NO: 1661 - DNA, 16 bp, Artificial sequence
  Synthetic oligonucleotide
ttgacttagtccgtgt

SEQ ID NO: 1662 - DNA, 16 bp, Artificial sequence
  Synthetic oligonucleotide
agctacatcaggctgg

SEQ ID NO: 1663 - DNA, 16 bp, Artificial sequence
  Synthetic oligonucleotide
tgcacatagactgtga

SEQ ID NO: 1664 - DNA, 16 bp, Artificial sequence
  Synthetic oligonucleotide
gactgctgagccaagc

SEQ ID NO: 1665 - DNA, 16 bp, Artificial sequence
  Synthetic oligonucleotide
agaaattgcagtgccc

SEQ ID NO: 1666 - DNA, 16 bp, Artificial sequence
  Synthetic oligonucleotide
ggtattaaactgccaa

SEQ ID NO: 1667 - DNA, 16 bp, Artificial sequence
  Synthetic oligonucleotide
tcactaaccaatatac

SEQ ID NO: 1668 - DNA, 16 bp, Artificial sequence
  Synthetic oligonucleotide
ataaatctgcaagagc

SEQ ID NO: 1669 - DNA, 16 bp, Artificial sequence
  Synthetic oligonucleotide
tctcatggtcaagacc

SEQ ID NO: 1670 - DNA, 16 bp, Artificial sequence
  Synthetic oligonucleotide
gactgctaggcttcac

SEQ ID NO: 1671 - DNA, 16 bp, Artificial sequence
  Synthetic oligonucleotide
cgctgctgcagtgtgc

SEQ ID NO: 1672 - DNA, 16 bp, Artificial sequence
  Synthetic oligonucleotide
ggcatccacgacttcg

SEQ ID NO: 1673 - DNA, 16 bp, Artificial sequence
  Synthetic oligonucleotide
aggcatccacgacttc

SEQ ID NO: 1674 - DNA, 16 bp, Artificial sequence
  Synthetic oligonucleotide
caaggcatccacgact

SEQ ID NO: 1675 - DNA, 16 bp, Artificial sequence
  Synthetic oligonucleotide
aacataccaaggcatc

SEQ ID NO: 1676 - DNA, 16 bp, Artificial sequence
  Synthetic oligonucleotide
ggaacataccaaggca

SEQ ID NO: 1677 - DNA, 16 bp, Artificial sequence
  Synthetic oligonucleotide
aggaacataccaaggc

SEQ ID NO: 1678 - DNA, 16 bp, Artificial sequence
  Synthetic oligonucleotide
gggataaggccactgt

SEQ ID NO: 1679 - DNA, 16 bp, Artificial sequence
  Synthetic oligonucleotide
agggataaggccactg

SEQ ID NO: 1680 - DNA, 16 bp, Artificial sequence
  Synthetic oligonucleotide
aaggagggataaggcc

SEQ ID NO: 1681 - DNA, 16 bp, Artificial sequence
  Synthetic oligonucleotide
acatatcgcacgcctc

SEQ ID NO: 1682 - DNA, 16 bp, Artificial sequence
  Synthetic oligonucleotide
cacatatcgcacgcct

SEQ ID NO: 1683 - DNA, 16 bp, Artificial sequence
  Synthetic oligonucleotide
ccacatatcgcacgcc

SEQ ID NO: 1684 - DNA, 16 bp, Artificial sequence
  Synthetic oligonucleotide
atccacatatcgcacg

SEQ ID NO: 1685 - DNA, 16 bp, Artificial sequence
  Synthetic oligonucleotide
ctccatccacatatcg

SEQ ID NO: 1686 - DNA, 16 bp, Artificial sequence
  Synthetic oligonucleotide
atcaatgaagggtacg

SEQ ID NO: 1687 - DNA, 16 bp, Artificial sequence
  Synthetic oligonucleotide
catcaatgaagggtac

SEQ ID NO: 1688 - DNA, 16 bp, Artificial sequence
  Synthetic oligonucleotide
atggaagatgccaatg

SEQ ID NO: 1689 - DNA, 16 bp, Artificial sequence
  Synthetic oligonucleotide
ggagatgagctggtgg

SEQ ID NO: 1690 - DNA, 16 bp, Artificial sequence
  Synthetic oligonucleotide
agactgagcttggtga

SEQ ID NO: 1691 - DNA, 16 bp, Artificial sequence
  Synthetic oligonucleotide
cctgaatgcatccaaa

SEQ ID NO: 1692 - DNA, 16 bp, Artificial sequence
  Synthetic oligonucleotide
atgctgagacgcaggt

SEQ ID NO: 1693 - DNA, 16 bp, Artificial sequence
  Synthetic oligonucleotide
aatggcagattccaca

SEQ ID NO: 1694 - DNA, 16 bp, Artificial sequence
  Synthetic oligonucleotide
ctctgaaagaatctgc

SEQ ID NO: 1695 - DNA, 16 bp, Artificial sequence
  Synthetic oligonucleotide
atgggaaactttagca

SEQ ID NO: 1696 - DNA, 16 bp, Artificial sequence
  Synthetic oligonucleotide
tgtcatttcccaacca

SEQ ID NO: 1697 - DNA, 16 bp, Artificial sequence
  Synthetic oligonucleotide
ttttagtagtcaaggt

SEQ ID NO: 1698 - DNA, 16 bp, Artificial sequence
  Synthetic oligonucleotide
gcttccttacattttt

SEQ ID NO: 1699 - DNA, 16 bp, Artificial sequence
  Synthetic oligonucleotide
taacatctcactgatt

SEQ ID NO: 1700 - DNA, 16 bp, Artificial sequence
  Synthetic oligonucleotide
ctagtgagaaacaaac

SEQ ID NO: 1701 - DNA, 16 bp, Artificial sequence
  Synthetic oligonucleotide
ttattgttgctaaacc

SEQ ID NO: 1702 - DNA, 16 bp, Artificial sequence
  Synthetic oligonucleotide
actttaggctcctggg

SEQ ID NO: 1703 - DNA, 16 bp, Artificial sequence
  Synthetic oligonucleotide
agccataaatcttggg

SEQ ID NO: 1704 - DNA, 16 bp, Artificial sequence
  Synthetic oligonucleotide
atgacatcatggcttc

SEQ ID NO: 1705 - DNA, 16 bp, Artificial sequence
  Synthetic oligonucleotide
ttattcaatgtggctt

SEQ ID NO: 1706 - DNA, 16 bp, Artificial sequence
  Synthetic oligonucleotide
gggctcaatgaaatta

SEQ ID NO: 1707 - DNA, 16 bp, Artificial sequence
  Synthetic oligonucleotide
cttagtatgaaatatc

SEQ ID NO: 1708 - DNA, 16 bp, Artificial sequence
  Synthetic oligonucleotide
tactgtctactatggg

SEQ ID NO: 1709 - DNA, 16 bp, Artificial sequence
  Synthetic oligonucleotide
cttacatccacgactt

SEQ ID NO: 1710 - DNA, 16 bp, Artificial sequence
  Synthetic oligonucleotide
cagtaactggtagctc

SEQ ID NO: 1711 - DNA, 16 bp, Artificial sequence
  Synthetic oligonucleotide
tgtttgattgtgcaga

SEQ ID NO: 1712 - DNA, 16 bp, Artificial sequence
  Synthetic oligonucleotide
cgccttttatttccgt

SEQ ID NO: 1713 - DNA, 16 bp, Artificial sequence
  Synthetic oligonucleotide
ataaccacagcctggg

SEQ ID NO: 1714 - DNA, 16 bp, Artificial sequence
  Synthetic oligonucleotide
ataagaatcatcttag

SEQ ID NO: 1715 - DNA, 16 bp, Artificial sequence
  Synthetic oligonucleotide
actaccgaacgcagtt

SEQ ID NO: 1716 - DNA, 16 bp, Artificial sequence
  Synthetic oligonucleotide
tagagtggtaaggcat

SEQ ID NO: 1717 - DNA, 16 bp, Artificial sequence
  Synthetic oligonucleotide
ggttggtgtacatggt

SEQ ID NO: 1718 - DNA, 16 bp, Artificial sequence
  Synthetic oligonucleotide
tcctgttagacagctt

SEQ ID NO: 1719 - DNA, 16 bp, Artificial sequence
  Synthetic oligonucleotide
ataaagcacacgggaa

SEQ ID NO: 1720 - DNA, 16 bp, Artificial sequence
  Synthetic oligonucleotide
taagagctgtctcctc

SEQ ID NO: 1721 - DNA, 16 bp, Artificial sequence
  Synthetic oligonucleotide
ctaacaaactttgcag

SEQ ID NO: 1722 - DNA, 16 bp, Artificial sequence
  Synthetic oligonucleotide
tgtcacccttccacgg

SEQ ID NO: 1723 - DNA, 16 bp, Artificial sequence
  Synthetic oligonucleotide
attggaagaccgcaga

SEQ ID NO: 1724 - DNA, 16 bp, Artificial sequence
  Synthetic oligonucleotide
ccgctagtaaatgctt

SEQ ID NO: 1725 - DNA, 16 bp, Artificial sequence
  Synthetic oligonucleotide
aactaaggcaaatctc

SEQ ID NO: 1726 - DNA, 16 bp, Artificial sequence
  Synthetic oligonucleotide
gagtcatgacatccca

SEQ ID NO: 1727 - DNA, 16 bp, Artificial sequence
  Synthetic oligonucleotide
gcagataaatacacat

SEQ ID NO: 1728 - DNA, 16 bp, Artificial sequence
  Synthetic oligonucleotide
tttcccatcgacacag

SEQ ID NO: 1729 - DNA, 16 bp, Artificial sequence
  Synthetic oligonucleotide
gctcctacttatcccc

SEQ ID NO: 1730 - DNA, 16 bp, Artificial sequence
  Synthetic oligonucleotide
tattgccaggtatctg

SEQ ID NO: 1731 - DNA, 16 bp, Artificial sequence
  Synthetic oligonucleotide
caatacatagcagagc

SEQ ID NO: 1732 - DNA, 16 bp, Artificial sequence
  Synthetic oligonucleotide
ttagtccgtgttcagg

SEQ ID NO: 1733 - DNA, 16 bp, Artificial sequence
  Synthetic oligonucleotide
gtagctggtttgtggg

SEQ ID NO: 1734 - DNA, 16 bp, Artificial sequence
  Synthetic oligonucleotide
catctcttagggcacc

SEQ ID NO: 1735 - DNA, 16 bp, Artificial sequence
  Synthetic oligonucleotide
gtttggaagtcgccat

SEQ ID NO: 1736 - DNA, 16 bp, Artificial sequence
  Synthetic oligonucleotide
aggaagcccaatcaag

SEQ ID NO: 1737 - DNA, 16 bp, Artificial sequence
  Synthetic oligonucleotide
cagattgagtctcctg

SEQ ID NO: 1738 - DNA, 16 bp, Artificial sequence
  Synthetic oligonucleotide
ttttacgatcatcatt

SEQ ID NO: 1739 - DNA, 16 bp, Artificial sequence
  Synthetic oligonucleotide
gcttagaaattgcagt

SEQ ID NO: 1740 - DNA, 16 bp, Artificial sequence
  Synthetic oligonucleotide
agggtaatattcagac

SEQ ID NO: 1741 - DNA, 16 bp, Artificial sequence
  Synthetic oligonucleotide
tgtagcagacagatca

SEQ ID NO: 1742 - DNA, 16 bp, Artificial sequence
  Synthetic oligonucleotide
tttaacagctcaggta

SEQ ID NO: 1743 - DNA, 16 bp, Artificial sequence
  Synthetic oligonucleotide
attctagacatggcca

SEQ ID NO: 1744 - DNA, 16 bp, Artificial sequence
  Synthetic oligonucleotide
aaccaatatactgaga

SEQ ID NO: 1745 - DNA, 16 bp, Artificial sequence
  Synthetic oligonucleotide
agacatatgacatttc

SEQ ID NO: 1746 - DNA, 16 bp, Artificial sequence
  Synthetic oligonucleotide
acatgacagactaact

SEQ ID NO: 1747 - DNA, 16 bp, Artificial sequence
  Synthetic oligonucleotide
catcaatgctgcactc

SEQ ID NO: 1748 - DNA, 16 bp, Artificial sequence
  Synthetic oligonucleotide
gttgaaggatggatgg

SEQ ID NO: 1749 - DNA, 16 bp, Artificial sequence
  Synthetic oligonucleotide
agagagatgcctattt

SEQ ID NO: 1750 - DNA, 16 bp, Artificial sequence
  Synthetic oligonucleotide
ggttccctgtgcagag

SEQ ID NO: 1751 - DNA, 16 bp, Artificial sequence
  Synthetic oligonucleotide
aagaacctgaatgcat

SEQ ID NO: 1752 - DNA, 16 bp, Artificial sequence
  Synthetic oligonucleotide
gatgctgagacgcagg

SEQ ID NO: 1753 - DNA, 16 bp, Artificial sequence
  Synthetic oligonucleotide
acagactcttctctag

SEQ ID NO: 1754 - DNA, 16 bp, Artificial sequence
  Synthetic oligonucleotide
cctctgaaagaatctg

SEQ ID NO: 1755 - DNA, 16 bp, Artificial sequence
  Synthetic oligonucleotide
gtagctgcacaaagat

SEQ ID NO: 1756 - DNA, 16 bp, Artificial sequence
  Synthetic oligonucleotide
tggtgtcatttcccaa

SEQ ID NO: 1757 - DNA, 16 bp, Artificial sequence
  Synthetic oligonucleotide
tttttagtagtcaagg

SEQ ID NO: 1758 - DNA, 16 bp, Artificial sequence
  Synthetic oligonucleotide
attcaacaggtaacaa

SEQ ID NO: 1759 - DNA, 16 bp, Artificial sequence
  Synthetic oligonucleotide
actaacatctcactga

SEQ ID NO: 1760 - DNA, 16 bp, Artificial sequence
  Synthetic oligonucleotide
agctagtgagaaacaa

SEQ ID NO: 1761 - DNA, 16 bp, Artificial sequence
  Synthetic oligonucleotide
cttttattgttgctaa

SEQ ID NO: 1762 - DNA, 16 bp, Artificial sequence
  Synthetic oligonucleotide
agtgtactttaggctc

SEQ ID NO: 1763 - DNA, 16 bp, Artificial sequence
  Synthetic oligonucleotide
cagccataaatcttgg

SEQ ID NO: 1764 - DNA, 16 bp, Artificial sequence
  Synthetic oligonucleotide
aatgacatcatggctt

SEQ ID NO: 1765 - DNA, 16 bp, Artificial sequence
  Synthetic oligonucleotide
tttattcaatgtggct

SEQ ID NO: 1766 - DNA, 16 bp, Artificial sequence
  Synthetic oligonucleotide
ttgggctcaatgaaat

SEQ ID NO: 1767 - DNA, 16 bp, Artificial sequence
  Synthetic oligonucleotide
gcttagtatgaaatat

SEQ ID NO: 1768 - DNA, 16 bp, Artificial sequence
  Synthetic oligonucleotide
gtactgtctactatgg

SEQ ID NO: 1769 - DNA, 16 bp, Artificial sequence
  Synthetic oligonucleotide
ctgcttacatccacga

SEQ ID NO: 1770 - DNA, 16 bp, Artificial sequence
  Synthetic oligonucleotide
acagtaactggtagct

SEQ ID NO: 1771 - DNA, 16 bp, Artificial sequence
  Synthetic oligonucleotide
ctgtttgattgtgcag

SEQ ID NO: 1772 - DNA, 16 bp, Artificial sequence
  Synthetic oligonucleotide
gcgccttttatttccg

SEQ ID NO: 1773 - DNA, 16 bp, Artificial sequence
  Synthetic oligonucleotide
cctgactatataacca

SEQ ID NO: 1774 - DNA, 16 bp, Artificial sequence
  Synthetic oligonucleotide
gaccgtgtttccaaat

SEQ ID NO: 1775 - DNA, 16 bp, Artificial sequence
  Synthetic oligonucleotide
aactaccgaacgcagt

SEQ ID NO: 1776 - DNA, 16 bp, Artificial sequence
  Synthetic oligonucleotide
ggtagagtggtaaggc

SEQ ID NO: 1777 - DNA, 16 bp, Artificial sequence
  Synthetic oligonucleotide
catagccttctttctt

SEQ ID NO: 1778 - DNA, 16 bp, Artificial sequence
  Synthetic oligonucleotide
aatcctgttagacagc

SEQ ID NO: 1779 - DNA, 16 bp, Artificial sequence
  Synthetic oligonucleotide
tataaagcacacggga

SEQ ID NO: 1780 - DNA, 16 bp, Artificial sequence
  Synthetic oligonucleotide
aataagagctgtctcc

SEQ ID NO: 1781 - DNA, 16 bp, Artificial sequence
  Synthetic oligonucleotide
ccctaacaaactttgc

SEQ ID NO: 1782 - DNA, 16 bp, Artificial sequence
  Synthetic oligonucleotide
aatgtcacccttccac

SEQ ID NO: 1783 - DNA, 16 bp, Artificial sequence
  Synthetic oligonucleotide
cattggaagaccgcag

SEQ ID NO: 1784 - DNA, 16 bp, Artificial sequence
  Synthetic oligonucleotide
accgctagtaaatgct

SEQ ID NO: 1785 - DNA, 16 bp, Artificial sequence
  Synthetic oligonucleotide
tagaactaaggcaaat

SEQ ID NO: 1786 - DNA, 16 bp, Artificial sequence
  Synthetic oligonucleotide
ggagtcatgacatccc

SEQ ID NO: 1787 - DNA, 16 bp, Artificial sequence
  Synthetic oligonucleotide
tgagcagataaataca

SEQ ID NO: 1788 - DNA, 16 bp, Artificial sequence
  Synthetic oligonucleotide
ctttcccatcgacaca

SEQ ID NO: 1789 - DNA, 16 bp, Artificial sequence
  Synthetic oligonucleotide
gcgctcctacttatcc

SEQ ID NO: 1790 - DNA, 16 bp, Artificial sequence
  Synthetic oligonucleotide
atattgccaggtatct

SEQ ID NO: 1791 - DNA, 16 bp, Artificial sequence
  Synthetic oligonucleotide
gtgttggtttataaca

SEQ ID NO: 1792 - DNA, 16 bp, Artificial sequence
  Synthetic oligonucleotide
acttagtccgtgttca

SEQ ID NO: 1793 - DNA, 16 bp, Artificial sequence
  Synthetic oligonucleotide
ctgtagctggtttgtg

SEQ ID NO: 1794 - DNA, 16 bp, Artificial sequence
  Synthetic oligonucleotide
ccatctcttagggcac

SEQ ID NO: 1795 - DNA, 16 bp, Artificial sequence
  Synthetic oligonucleotide
tgtttggaagtcgcca

SEQ ID NO: 1796 - DNA, 16 bp, Artificial sequence
  Synthetic oligonucleotide
atcagaggaagcccaa

SEQ ID NO: 1797 - DNA, 16 bp, Artificial sequence
  Synthetic oligonucleotide
accagattgagtctcc

SEQ ID NO: 1798 - DNA, 16 bp, Artificial sequence
  Synthetic oligonucleotide
ctctaattttacgatc

SEQ ID NO: 1799 - DNA, 16 bp, Artificial sequence
  Synthetic oligonucleotide
agcttagaaattgcag

SEQ ID NO: 1800 - DNA, 16 bp, Artificial sequence
  Synthetic oligonucleotide
cagggtaatattcaga

SEQ ID NO: 1801 - DNA, 16 bp, Artificial sequence
  Synthetic oligonucleotide
tttgtagcagacagat

SEQ ID NO: 1802 - DNA, 16 bp, Artificial sequence
  Synthetic oligonucleotide
ttttaacagctcaggt

SEQ ID NO: 1803 - DNA, 16 bp, Artificial sequence
  Synthetic oligonucleotide
aattctagacatggcc

SEQ ID NO: 1804 - DNA, 16 bp, Artificial sequence
  Synthetic oligonucleotide
taaccaatatactgag

SEQ ID NO: 1805 - DNA, 16 bp, Artificial sequence
  Synthetic oligonucleotide
cgcaaaaagacaacga

SEQ ID NO: 1806 - DNA, 16 bp, Artificial sequence
  Synthetic oligonucleotide
gacatgacagactaac

SEQ ID NO: 1807 - DNA, 16 bp, Artificial sequence
  Synthetic oligonucleotide
ccatcaatgctgcact

SEQ ID NO: 1808 - DNA, 16 bp, Artificial sequence
  Synthetic oligonucleotide
agttgaaggatggatg

SEQ ID NO: 1809 - DNA, 16 bp, Artificial sequence
  Synthetic oligonucleotide
ctggtaagagagatgc

SEQ ID NO: 1810 - DNA, 16 bp, Artificial sequence
  Synthetic oligonucleotide
gaggttccctgtgcag

SEQ ID NO: 1811 - DNA, 16 bp, Artificial sequence
  Synthetic oligonucleotide
caagaacctgaatgca

SEQ ID NO: 1812 - DNA, 16 bp, Artificial sequence
  Synthetic oligonucleotide
aggatgctgagacgca

SEQ ID NO: 1813 - DNA, 16 bp, Artificial sequence
  Synthetic oligonucleotide
tcacagactcttctct

SEQ ID NO: 1814 - DNA, 16 bp, Artificial sequence
  Synthetic oligonucleotide
acctctgaaagaatct

SEQ ID NO: 1815 - DNA, 16 bp, Artificial sequence
  Synthetic oligonucleotide
aggtagctgcacaaag

SEQ ID NO: 1816 - DNA, 16 bp, Artificial sequence
  Synthetic oligonucleotide
cagtctgaccattaat

SEQ ID NO: 1817 - DNA, 16 bp, Artificial sequence
  Synthetic oligonucleotide
gaaaaaggtgttctaa

SEQ ID NO: 1818 - DNA, 16 bp, Artificial sequence
  Synthetic oligonucleotide
aaattcaacaggtaac

SEQ ID NO: 1819 - DNA, 16 bp, Artificial sequence
  Synthetic oligonucleotide
ttctactaacatctca

SEQ ID NO: 1820 - DNA, 16 bp, Artificial sequence
  Synthetic oligonucleotide
atgatcatgtggcggt

SEQ ID NO: 1821 - DNA, 16 bp, Artificial sequence
  Synthetic oligonucleotide
acttttattgttgcta

SEQ ID NO: 1822 - DNA, 16 bp, Artificial sequence
  Synthetic oligonucleotide
gagtgtactttaggct

SEQ ID NO: 1823 - DNA, 16 bp, Artificial sequence
  Synthetic oligonucleotide
ctatcaggtgcaggag

SEQ ID NO: 1824 - DNA, 16 bp, Artificial sequence
  Synthetic oligonucleotide
caatgacatcatggct

SEQ ID NO: 1825 - DNA, 16 bp, Artificial sequence
  Synthetic oligonucleotide
tactttattcaatgtg

SEQ ID NO: 1826 - DNA, 16 bp, Artificial sequence
  Synthetic oligonucleotide
attgggctcaatgaaa

SEQ ID NO: 1827 - DNA, 16 bp, Artificial sequence
  Synthetic oligonucleotide
tggcttagtatgaaat

SEQ ID NO: 1828 - DNA, 16 bp, Artificial sequence
  Synthetic oligonucleotide
tttggcaaggccagaa

SEQ ID NO: 1829 - DNA, 16 bp, Artificial sequence
  Synthetic oligonucleotide
gcatagaggaagctcg

SEQ ID NO: 1830 - DNA, 16 bp, Artificial sequence
  Synthetic oligonucleotide
gacagtaactggtagc

SEQ ID NO: 1831 - DNA, 16 bp, Artificial sequence
  Synthetic oligonucleotide
ttctgtttgattgtgc

SEQ ID NO: 1832 - DNA, 16 bp, Artificial sequence
  Synthetic oligonucleotide
aggtctgggtatatgt

SEQ ID NO: 1833 - DNA, 16 bp, Artificial sequence
  Synthetic oligonucleotide
ccctgactatataacc

SEQ ID NO: 1834 - DNA, 16 bp, Artificial sequence
  Synthetic oligonucleotide
ttgaccgtgtttccaa

SEQ ID NO: 1835 - DNA, 16 bp, Artificial sequence
  Synthetic oligonucleotide
aaactaccgaacgcag

SEQ ID NO: 1836 - DNA, 16 bp, Artificial sequence
  Synthetic oligonucleotide
tggtagagtggtaagg

SEQ ID NO: 1837 - DNA, 16 bp, Artificial sequence
  Synthetic oligonucleotide
gcatagccttctttct

SEQ ID NO: 1838 - DNA, 16 bp, Artificial sequence
  Synthetic oligonucleotide
caatcctgttagacag

SEQ ID NO: 1839 - DNA, 16 bp, Artificial sequence
  Synthetic oligonucleotide
ttataaagcacacggg

SEQ ID NO: 1840 - DNA, 16 bp, Artificial sequence
  Synthetic oligonucleotide
caataagagctgtctc

SEQ ID NO: 1841 - DNA, 16 bp, Artificial sequence
  Synthetic oligonucleotide
tgcaggcaccccagca

SEQ ID NO: 1842 - DNA, 16 bp, Artificial sequence
  Synthetic oligonucleotide
gaatgtcacccttcca

SEQ ID NO: 1843 - DNA, 16 bp, Artificial sequence
  Synthetic oligonucleotide
tcattggaagaccgca

SEQ ID NO: 1844 - DNA, 16 bp, Artificial sequence
  Synthetic oligonucleotide
gaccgctagtaaatgc

SEQ ID NO: 1845 - DNA, 16 bp, Artificial sequence
  Synthetic oligonucleotide
ttagaactaaggcaaa

SEQ ID NO: 1846 - DNA, 16 bp, Artificial sequence
  Synthetic oligonucleotide
tggagtcatgacatcc

SEQ ID NO: 1847 - DNA, 16 bp, Artificial sequence
  Synthetic oligonucleotide
ctgagcagataaatac

SEQ ID NO: 1848 - DNA, 16 bp, Artificial sequence
  Synthetic oligonucleotide
agtcttaatgtggatt

SEQ ID NO: 1849 - DNA, 16 bp, Artificial sequence
  Synthetic oligonucleotide
agtgtccccatcccca

SEQ ID NO: 1850 - DNA, 16 bp, Artificial sequence
  Synthetic oligonucleotide
aatattgccaggtatc

SEQ ID NO: 1851 - DNA, 16 bp, Artificial sequence
  Synthetic oligonucleotide
tagtgttggtttataa

SEQ ID NO: 1852 - DNA, 16 bp, Artificial sequence
  Synthetic oligonucleotide
tgacttagtccgtgtt

SEQ ID NO: 1853 - DNA, 16 bp, Artificial sequence
  Synthetic oligonucleotide
tctgtagctggtttgt

SEQ ID NO: 1854 - DNA, 16 bp, Artificial sequence
  Synthetic oligonucleotide
tcccatctcttagggc

SEQ ID NO: 1855 - DNA, 16 bp, Artificial sequence
  Synthetic oligonucleotide
tatgtttggaagtcgc

SEQ ID NO: 1856 - DNA, 16 bp, Artificial sequence
  Synthetic oligonucleotide
aatcagaggaagccca

SEQ ID NO: 1857 - DNA, 16 bp, Artificial sequence
  Synthetic oligonucleotide
aaccagattgagtctc

SEQ ID NO: 1858 - DNA, 16 bp, Artificial sequence
  Synthetic oligonucleotide
tctctaattttacgat

SEQ ID NO: 1859 - DNA, 16 bp, Artificial sequence
  Synthetic oligonucleotide
cagcttagaaattgca

SEQ ID NO: 1860 - DNA, 16 bp, Artificial sequence
  Synthetic oligonucleotide
ccagggtaatattcag

SEQ ID NO: 1861 - DNA, 16 bp, Artificial sequence
  Synthetic oligonucleotide
ctttgtagcagacaga

SEQ ID NO: 1862 - DNA, 16 bp, Artificial sequence
  Synthetic oligonucleotide
tattttaacagctcag

SEQ ID NO: 1863 - DNA, 16 bp, Artificial sequence
  Synthetic oligonucleotide
tgcaattctagacatg

SEQ ID NO: 1864 - DNA, 16 bp, Artificial sequence
  Synthetic oligonucleotide
actaaccaatatactg

SEQ ID NO: 1865 - DNA, 16 bp, Artificial sequence
  Synthetic oligonucleotide
caacagattactggac

SEQ ID NO: 1866 - DNA, 16 bp, Artificial sequence
  Synthetic oligonucleotide
aggacatgacagacta

SEQ ID NO: 1867 - DNA, 16 bp, Artificial sequence
  Synthetic oligonucleotide
accatcaatgctgcac

SEQ ID NO: 1868 - DNA, 16 bp, Artificial sequence
  Synthetic oligonucleotide
aagttgaaggatggat

SEQ ID NO: 1869 - DNA, 16 bp, Artificial sequence
  Synthetic oligonucleotide
ttggcatcaatgaagg

SEQ ID NO: 1870 - DNA, 16 bp, Artificial sequence
  Synthetic oligonucleotide
agaggttccctgtgca

SEQ ID NO: 1871 - DNA, 16 bp, Artificial sequence
  Synthetic oligonucleotide
ccaagaacctgaatgc

SEQ ID NO: 1872 - DNA, 16 bp, Artificial sequence
  Synthetic oligonucleotide
ggcaggatgctgagac

SEQ ID NO: 1873 - DNA, 16 bp, Artificial sequence
  Synthetic oligonucleotide
ctcacagactcttctc

SEQ ID NO: 1874 - DNA, 16 bp, Artificial sequence
  Synthetic oligonucleotide
gcacctctgaaagaat

SEQ ID NO: 1875 - DNA, 16 bp, Artificial sequence
  Synthetic oligonucleotide
ggaggtagctgcacaa

SEQ ID NO: 1876 - DNA, 16 bp, Artificial sequence
  Synthetic oligonucleotide
acagtctgaccattaa

SEQ ID NO: 1877 - DNA, 16 bp, Artificial sequence
  Synthetic oligonucleotide
ttaggtgaaaaaggtg

SEQ ID NO: 1878 - DNA, 16 bp, Artificial sequence
  Synthetic oligonucleotide
tgattcacataataca

SEQ ID NO: 1879 - DNA, 16 bp, Artificial sequence
  Synthetic oligonucleotide
tattctactaacatct

SEQ ID NO: 1880 - DNA, 16 bp, Artificial sequence
  Synthetic oligonucleotide
aatgatcatgtggcgg

SEQ ID NO: 1881 - DNA, 16 bp, Artificial sequence
  Synthetic oligonucleotide
tgacttttattgttgc

SEQ ID NO: 1882 - DNA, 16 bp, Artificial sequence
  Synthetic oligonucleotide
tgagtgtactttaggc

SEQ ID NO: 1883 - DNA, 16 bp, Artificial sequence
  Synthetic oligonucleotide
atgctatcaggtgcag

SEQ ID NO: 1884 - DNA, 16 bp, Artificial sequence
  Synthetic oligonucleotide
gcacaatgacatcatg

SEQ ID NO: 1885 - DNA, 16 bp, Artificial sequence
  Synthetic oligonucleotide
ttactttattcaatgt

SEQ ID NO: 1886 - DNA, 16 bp, Artificial sequence
  Synthetic oligonucleotide
tattgggctcaatgaa

SEQ ID NO: 1887 - DNA, 16 bp, Artificial sequence
  Synthetic oligonucleotide
tatgggagccacatgt

SEQ ID NO: 1888 - DNA, 16 bp, Artificial sequence
  Synthetic oligonucleotide
cttttggcaaggccag

SEQ ID NO: 1889 - DNA, 16 bp, Artificial sequence
  Synthetic oligonucleotide
ttaaacagaggatgca

SEQ ID NO: 1890 - DNA, 16 bp, Artificial sequence
  Synthetic oligonucleotide
agacagtaactggtag

SEQ ID NO: 1891 - DNA, 16 bp, Artificial sequence
  Synthetic oligonucleotide
tccgttaaccatcaag

SEQ ID NO: 1892 - DNA, 16 bp, Artificial sequence
  Synthetic oligonucleotide
ttaggtctgggtatat

SEQ ID NO: 1893 - DNA, 16 bp, Artificial sequence
  Synthetic oligonucleotide
cccctgactatataac

SEQ ID NO: 1894 - DNA, 16 bp, Artificial sequence
  Synthetic oligonucleotide
cttgaccgtgtttcca

SEQ ID NO: 1895 - DNA, 16 bp, Artificial sequence
  Synthetic oligonucleotide
ttaaactaccgaacgc

SEQ ID NO: 1896 - DNA, 16 bp, Artificial sequence
  Synthetic oligonucleotide
atggtagagtggtaag

SEQ ID NO: 1897 - DNA, 16 bp, Artificial sequence
  Synthetic oligonucleotide
attgcatagccttctt

SEQ ID NO: 1898 - DNA, 16 bp, Artificial sequence
  Synthetic oligonucleotide
ccaatcctgttagaca

SEQ ID NO: 1899 - DNA, 16 bp, Artificial sequence
  Synthetic oligonucleotide
ctgcttataaagcaca

SEQ ID NO: 1900 - DNA, 16 bp, Artificial sequence
  Synthetic oligonucleotide
acaataagagctgtct

SEQ ID NO: 1901 - DNA, 16 bp, Artificial sequence
  Synthetic oligonucleotide
tttgcaggcaccccag

SEQ ID NO: 1902 - DNA, 16 bp, Artificial sequence
  Synthetic oligonucleotide
tgaatgtcacccttcc

SEQ ID NO: 1903 - DNA, 16 bp, Artificial sequence
  Synthetic oligonucleotide
gcatcattggaagacc

SEQ ID NO: 1904 - DNA, 16 bp, Artificial sequence
  Synthetic oligonucleotide
accaagaccgctagta

SEQ ID NO: 1905 - DNA, 16 bp, Artificial sequence
  Synthetic oligonucleotide
tgttagaactaaggca

SEQ ID NO: 1906 - DNA, 16 bp, Artificial sequence
  Synthetic oligonucleotide
cctggagtcatgacat

SEQ ID NO: 1907 - DNA, 16 bp, Artificial sequence
  Synthetic oligonucleotide
tctgagcagataaata

SEQ ID NO: 1908 - DNA, 16 bp, Artificial sequence
  Synthetic oligonucleotide
aagtcttaatgtggat

SEQ ID NO: 1909 - DNA, 16 bp, Artificial sequence
  Synthetic oligonucleotide
ttagtgtccccatccc

SEQ ID NO: 1910 - DNA, 16 bp, Artificial sequence
  Synthetic oligonucleotide
gaatattgccaggtat

SEQ ID NO: 1911 - DNA, 16 bp, Artificial sequence
  Synthetic oligonucleotide
ttagtgttggtttata

SEQ ID NO: 1912 - DNA, 16 bp, Artificial sequence
  Synthetic oligonucleotide
tttgacttagtccgtg

SEQ ID NO: 1913 - DNA, 16 bp, Artificial sequence
  Synthetic oligonucleotide
ctctgtagctggtttg

SEQ ID NO: 1914 - DNA, 16 bp, Artificial sequence
  Synthetic oligonucleotide
ttgatagtgaatgtgt

SEQ ID NO: 1915 - DNA, 16 bp, Artificial sequence
  Synthetic oligonucleotide
atatgtttggaagtcg

SEQ ID NO: 1916 - DNA, 16 bp, Artificial sequence
  Synthetic oligonucleotide
caatcagaggaagccc

SEQ ID NO: 1917 - DNA, 16 bp, Artificial sequence
  Synthetic oligonucleotide
taaccagattgagtct

SEQ ID NO: 1918 - DNA, 16 bp, Artificial sequence
  Synthetic oligonucleotide
gtctctaattttacga

SEQ ID NO: 1919 - DNA, 16 bp, Artificial sequence
  Synthetic oligonucleotide
acagcttagaaattgc

SEQ ID NO: 1920 - DNA, 16 bp, Artificial sequence
  Synthetic oligonucleotide
ctgtattagctcaata

SEQ ID NO: 1921 - DNA, 16 bp, Artificial sequence
  Synthetic oligonucleotide
tctttgtagcagacag

SEQ ID NO: 1922 - DNA, 16 bp, Artificial sequence
  Synthetic oligonucleotide
ttattttaacagctca

SEQ ID NO: 1923 - DNA, 16 bp, Artificial sequence
  Synthetic oligonucleotide
ctgcaattctagacat

SEQ ID NO: 1924 - DNA, 16 bp, Artificial sequence
  Synthetic oligonucleotide
catttcagagtataag

SEQ ID NO: 1925 - DNA, 16 bp, Artificial sequence
  Synthetic oligonucleotide
gatgtgagtgaaataa

SEQ ID NO: 1926 - DNA, 16 bp, Artificial sequence
  Synthetic oligonucleotide
aaggacatgacagact

SEQ ID NO: 1927 - DNA, 16 bp, Artificial sequence
  Synthetic oligonucleotide
ccaccatcaatgctgc

SEQ ID NO: 1928 - DNA, 16 bp, Artificial sequence
  Synthetic oligonucleotide
tttggcatcaatgaag

SEQ ID NO: 1929 - DNA, 16 bp, Artificial sequence
  Synthetic oligonucleotide
tagaggttccctgtgc

SEQ ID NO: 1930 - DNA, 16 bp, Artificial sequence
  Synthetic oligonucleotide
gggcaggatgctgaga

SEQ ID NO: 1931 - DNA, 16 bp, Artificial sequence
  Synthetic oligonucleotide
actcacagactcttct

SEQ ID NO: 1932 - DNA, 16 bp, Artificial sequence
  Synthetic oligonucleotide
agcacctctgaaagaa

SEQ ID NO: 1933 - DNA, 16 bp, Artificial sequence
  Synthetic oligonucleotide
cggaggtagctgcaca

SEQ ID NO: 1934 - DNA, 16 bp, Artificial sequence
  Synthetic oligonucleotide
attctaagaacctcat

SEQ ID NO: 1935 - DNA, 16 bp, Artificial sequence
  Synthetic oligonucleotide
aaacaaaccctccgtc

SEQ ID NO: 1936 - DNA, 16 bp, Artificial sequence
  Synthetic oligonucleotide
aaatgatcatgtggcg

SEQ ID NO: 1937 - DNA, 16 bp, Artificial sequence
  Synthetic oligonucleotide
ctgacttttattgttg

SEQ ID NO: 1938 - DNA, 16 bp, Artificial sequence
  Synthetic oligonucleotide
gtgagtgtactttagg

SEQ ID NO: 1939 - DNA, 16 bp, Artificial sequence
  Synthetic oligonucleotide
aatgctatcaggtgca

SEQ ID NO: 1940 - DNA, 16 bp, Artificial sequence
  Synthetic oligonucleotide
tgcacaatgacatcat

SEQ ID NO: 1941 - DNA, 16 bp, Artificial sequence
  Synthetic oligonucleotide
ctacctgtgtctttta

SEQ ID NO: 1942 - DNA, 16 bp, Artificial sequence
  Synthetic oligonucleotide
atatattgggctcaat

SEQ ID NO: 1943 - DNA, 16 bp, Artificial sequence
  Synthetic oligonucleotide
acttttggcaaggcca

SEQ ID NO: 1944 - DNA, 16 bp, Artificial sequence
  Synthetic oligonucleotide
ccgcaaacaaggttaa

SEQ ID NO: 1945 - DNA, 16 bp, Artificial sequence
  Synthetic oligonucleotide
tttaggtctgggtata

SEQ ID NO: 1946 - DNA, 16 bp, Artificial sequence
  Synthetic oligonucleotide
ccccctgactatataa

SEQ ID NO: 1947 - DNA, 16 bp, Artificial sequence
  Synthetic oligonucleotide
tcttgaccgtgtttcc

SEQ ID NO: 1948 - DNA, 16 bp, Artificial sequence
  Synthetic oligonucleotide
gcattgcatagccttc

SEQ ID NO: 1949 - DNA, 16 bp, Artificial sequence
  Synthetic oligonucleotide
aaccaatcctgttaga

SEQ ID NO: 1950 - DNA, 16 bp, Artificial sequence
  Synthetic oligonucleotide
tctgcttataaagcac

SEQ ID NO: 1951 - DNA, 16 bp, Artificial sequence
  Synthetic oligonucleotide
ctttgcaggcacccca

SEQ ID NO: 1952 - DNA, 16 bp, Artificial sequence
  Synthetic oligonucleotide
cttgaatgtcaccctt

SEQ ID NO: 1953 - DNA, 16 bp, Artificial sequence
  Synthetic oligonucleotide
ttaccaagaccgctag

SEQ ID NO: 1954 - DNA, 16 bp, Artificial sequence
  Synthetic oligonucleotide
acttttagtattaaag

SEQ ID NO: 1955 - DNA, 16 bp, Artificial sequence
  Synthetic oligonucleotide
aaagtcttaatgtgga

SEQ ID NO: 1956 - DNA, 16 bp, Artificial sequence
  Synthetic oligonucleotide
cttagtgtccccatcc

SEQ ID NO: 1957 - DNA, 16 bp, Artificial sequence
  Synthetic oligonucleotide
gttagtgttggtttat

SEQ ID NO: 1958 - DNA, 16 bp, Artificial sequence
  Synthetic oligonucleotide
gtttgacttagtccgt

SEQ ID NO: 1959 - DNA, 16 bp, Artificial sequence
  Synthetic oligonucleotide
aatatgtttggaagtc

SEQ ID NO: 1960 - DNA, 16 bp, Artificial sequence
  Synthetic oligonucleotide
agtaaccagattgagt

SEQ ID NO: 1961 - DNA, 16 bp, Artificial sequence
  Synthetic oligonucleotide
cacagcttagaaattg

SEQ ID NO: 1962 - DNA, 16 bp, Artificial sequence
  Synthetic oligonucleotide
cctgtattagctcaat

SEQ ID NO: 1963 - DNA, 16 bp, Artificial sequence
  Synthetic oligonucleotide
cctctttgtagcagac

SEQ ID NO: 1964 - DNA, 16 bp, Artificial sequence
  Synthetic oligonucleotide
ggttattttaacagct

SEQ ID NO: 1965 - DNA, 16 bp, Artificial sequence
  Synthetic oligonucleotide
acctgcaattctagac

SEQ ID NO: 1966 - DNA, 16 bp, Artificial sequence
  Synthetic oligonucleotide
aagatgtgagtgaaat

SEQ ID NO: 1967 - DNA, 16 bp, Artificial sequence
  Synthetic oligonucleotide
aaaggacatgacagac

SEQ ID NO: 1968 - DNA, 16 bp, Artificial sequence
  Synthetic oligonucleotide
gaggaacttgcttaag

SEQ ID NO: 1969 - DNA, 16 bp, Artificial sequence
  Synthetic oligonucleotide
ttttggcatcaatgaa

SEQ ID NO: 1970 - DNA, 16 bp, Artificial sequence
  Synthetic oligonucleotide
tctagcagctcatctc

SEQ ID NO: 1971 - DNA, 16 bp, Artificial sequence
  Synthetic oligonucleotide
ttagcacctctgaaag

SEQ ID NO: 1972 - DNA, 16 bp, Artificial sequence
  Synthetic oligonucleotide
atgtattagagttaag

SEQ ID NO: 1973 - DNA, 16 bp, Artificial sequence
  Synthetic oligonucleotide
cattctaagaacctca

SEQ ID NO: 1974 - DNA, 16 bp, Artificial sequence
  Synthetic oligonucleotide
ttagttaggtgaaaaa

SEQ ID NO: 1975 - DNA, 16 bp, Artificial sequence
  Synthetic oligonucleotide
cactgattcacataat

SEQ ID NO: 1976 - DNA, 16 bp, Artificial sequence
  Synthetic oligonucleotide
tgcaaatgatcatgtg

SEQ ID NO: 1977 - DNA, 16 bp, Artificial sequence
  Synthetic oligonucleotide
gctgacttttattgtt

SEQ ID NO: 1978 - DNA, 16 bp, Artificial sequence
  Synthetic oligonucleotide
agtgagtgtactttag

SEQ ID NO: 1979 - DNA, 16 bp, Artificial sequence
  Synthetic oligonucleotide
ttaatgctatcaggtg

SEQ ID NO: 1980 - DNA, 16 bp, Artificial sequence
  Synthetic oligonucleotide
atgcacaatgacatca

SEQ ID NO: 1981 - DNA, 16 bp, Artificial sequence
  Synthetic oligonucleotide
attctacctgtgtctt

SEQ ID NO: 1982 - DNA, 16 bp, Artificial sequence
  Synthetic oligonucleotide
ttggatatattgggct

SEQ ID NO: 1983 - DNA, 16 bp, Artificial sequence
  Synthetic oligonucleotide
tacttttggcaaggcc

SEQ ID NO: 1984 - DNA, 16 bp, Artificial sequence
  Synthetic oligonucleotide
gcacagagtaggttaa

SEQ ID NO: 1985 - DNA, 16 bp, Artificial sequence
  Synthetic oligonucleotide
tggtagctcctggcaa

SEQ ID NO: 1986 - DNA, 16 bp, Artificial sequence
  Synthetic oligonucleotide
tttccgttaaccatca

SEQ ID NO: 1987 - DNA, 16 bp, Artificial sequence
  Synthetic oligonucleotide
catcttagtggctggg

SEQ ID NO: 1988 - DNA, 16 bp, Artificial sequence
  Synthetic oligonucleotide
gttcttgaccgtgttt

SEQ ID NO: 1989 - DNA, 16 bp, Artificial sequence
  Synthetic oligonucleotide
ggttaaactaccgaac

SEQ ID NO: 1990 - DNA, 16 bp, Artificial sequence
  Synthetic oligonucleotide
catggtctgcaaattt

SEQ ID NO: 1991 - DNA, 16 bp, Artificial sequence
  Synthetic oligonucleotide
aaaccaatcctgttag

SEQ ID NO: 1992 - DNA, 16 bp, Artificial sequence
  Synthetic oligonucleotide
atctgcttataaagca

SEQ ID NO: 1993 - DNA, 16 bp, Artificial sequence
  Synthetic oligonucleotide
actttgcaggcacccc

SEQ ID NO: 1994 - DNA, 16 bp, Artificial sequence
  Synthetic oligonucleotide
gcttgaatgtcaccct

SEQ ID NO: 1995 - DNA, 16 bp, Artificial sequence
  Synthetic oligonucleotide
tttaccaagaccgcta

SEQ ID NO: 1996 - DNA, 16 bp, Artificial sequence
  Synthetic oligonucleotide
caacttttagtattaa

SEQ ID NO: 1997 - DNA, 16 bp, Artificial sequence
  Synthetic oligonucleotide
tcgacacagcatcacc

SEQ ID NO: 1998 - DNA, 16 bp, Artificial sequence
  Synthetic oligonucleotide
tcttagtgtccccatc

SEQ ID NO: 1999 - DNA, 16 bp, Artificial sequence
  Synthetic oligonucleotide
ttaggaatattgccag

SEQ ID NO: 2000 - DNA, 16 bp, Artificial sequence
  Synthetic oligonucleotide
gggttagtgttggttt

SEQ ID NO: 2001 - DNA, 16 bp, Artificial sequence
  Synthetic oligonucleotide
tagggcacctcaagaa

SEQ ID NO: 2002 - DNA, 16 bp, Artificial sequence
  Synthetic oligonucleotide
caaatatgtttggaag

SEQ ID NO: 2003 - DNA, 16 bp, Artificial sequence
  Synthetic oligonucleotide
cccaatcagaggaagc

SEQ ID NO: 2004 - DNA, 16 bp, Artificial sequence
  Synthetic oligonucleotide
tcatcattattacctg

SEQ ID NO: 2005 - DNA, 16 bp, Artificial sequence
  Synthetic oligonucleotide
ttcagaccagggtaat

SEQ ID NO: 2006 - DNA, 16 bp, Artificial sequence
  Synthetic oligonucleotide
gcctgtattagctcaa

SEQ ID NO: 2007 - DNA, 16 bp, Artificial sequence
  Synthetic oligonucleotide
gcctctttgtagcaga

SEQ ID NO: 2008 - DNA, 16 bp, Artificial sequence
  Synthetic oligonucleotide
tactgagaggaaatga

SEQ ID NO: 2009 - DNA, 16 bp, Artificial sequence
  Synthetic oligonucleotide
tgtaaagatgtgagtg

SEQ ID NO: 2010 - DNA, 16 bp, Artificial sequence
  Synthetic oligonucleotide
tcaaaggacatgacag

SEQ ID NO: 2011 - DNA, 16 bp, Artificial sequence
  Synthetic oligonucleotide
gagcaacgaggaagga

SEQ ID NO: 2012 - DNA, 16 bp, Artificial sequence
  Synthetic oligonucleotide
cctgtcggaggaactt

SEQ ID NO: 2013 - DNA, 16 bp, Artificial sequence
  Synthetic oligonucleotide
ttgttttggcatcaat

SEQ ID NO: 2014 - DNA, 16 bp, Artificial sequence
  Synthetic oligonucleotide
aatgcatccaaatatc

SEQ ID NO: 2015 - DNA, 16 bp, Artificial sequence
  Synthetic oligonucleotide
tggtctagcagctcat

SEQ ID NO: 2016 - DNA, 16 bp, Artificial sequence
  Synthetic oligonucleotide
ttacataagacattat

SEQ ID NO: 2017 - DNA, 16 bp, Artificial sequence
  Synthetic oligonucleotide
ctcaagtgactcacag

SEQ ID NO: 2018 - DNA, 16 bp, Artificial sequence
  Synthetic oligonucleotide
tttagcacctctgaaa

SEQ ID NO: 2019 - DNA, 16 bp, Artificial sequence
  Synthetic oligonucleotide
tttcccaaccagctga

SEQ ID NO: 2020 - DNA, 16 bp, Artificial sequence
  Synthetic oligonucleotide
tcatctttgcagacca

SEQ ID NO: 2021 - DNA, 16 bp, Artificial sequence
  Synthetic oligonucleotide
tttagttaggtgaaaa

SEQ ID NO: 2022 - DNA, 16 bp, Artificial sequence
  Synthetic oligonucleotide
tcactgattcacataa

SEQ ID NO: 2023 - DNA, 16 bp, Artificial sequence
  Synthetic oligonucleotide
gagaaacaaaccctcc

SEQ ID NO: 2024 - DNA, 16 bp, Artificial sequence
  Synthetic oligonucleotide
gtgcaaatgatcatgt

SEQ ID NO: 2025 - DNA, 16 bp, Artificial sequence
  Synthetic oligonucleotide
aagctgacttttattg

SEQ ID NO: 2026 - DNA, 16 bp, Artificial sequence
  Synthetic oligonucleotide
tcttgggatgcacagg

SEQ ID NO: 2027 - DNA, 16 bp, Artificial sequence
  Synthetic oligonucleotide
ccttaatgctatcagg

SEQ ID NO: 2028 - DNA, 16 bp, Artificial sequence
  Synthetic oligonucleotide
aatgcacaatgacatc

SEQ ID NO: 2029 - DNA, 16 bp, Artificial sequence
  Synthetic oligonucleotide
aattctacctgtgtct

SEQ ID NO: 2030 - DNA, 16 bp, Artificial sequence
  Synthetic oligonucleotide
tttggatatattgggc

SEQ ID NO: 2031 - DNA, 16 bp, Artificial sequence
  Synthetic oligonucleotide
ctactatgggagccac

SEQ ID NO: 2032 - DNA, 16 bp, Artificial sequence
  Synthetic oligonucleotide
taatacttttggcaag

SEQ ID NO: 2033 - DNA, 16 bp, Artificial sequence
  Synthetic oligonucleotide
ttataggcgagagcac

SEQ ID NO: 2034 - DNA, 16 bp, Artificial sequence
  Synthetic oligonucleotide
ctggtagctcctggca

SEQ ID NO: 2035 - DNA, 16 bp, Artificial sequence
  Synthetic oligonucleotide
gattgtgcagacagta

SEQ ID NO: 2036 - DNA, 16 bp, Artificial sequence
  Synthetic oligonucleotide
atttccgttaaccatc

SEQ ID NO: 2037 - DNA, 16 bp, Artificial sequence
  Synthetic oligonucleotide
tgagttttaggtctgg

SEQ ID NO: 2038 - DNA, 16 bp, Artificial sequence
  Synthetic oligonucleotide
atcatcttagtggctg

SEQ ID NO: 2039 - DNA, 16 bp, Artificial sequence
  Synthetic oligonucleotide
tgttcttgaccgtgtt

SEQ ID NO: 2040 - DNA, 16 bp, Artificial sequence
  Synthetic oligonucleotide
aaggttaaactaccga

SEQ ID NO: 2041 - DNA, 16 bp, Artificial sequence
  Synthetic oligonucleotide
tacatggtctgcaaat

SEQ ID NO: 2042 - DNA, 16 bp, Artificial sequence
  Synthetic oligonucleotide
cattgcattgcatagc

SEQ ID NO: 2043 - DNA, 16 bp, Artificial sequence
  Synthetic oligonucleotide
aaaaccaatcctgtta

SEQ ID NO: 2044 - DNA, 16 bp, Artificial sequence
  Synthetic oligonucleotide
catctgcttataaagc

SEQ ID NO: 2045 - DNA, 16 bp, Artificial sequence
  Synthetic oligonucleotide
aaactttgcagcctat

SEQ ID NO: 2046 - DNA, 16 bp, Artificial sequence
  Synthetic oligonucleotide
agactttgcaggcacc

SEQ ID NO: 2047 - DNA, 16 bp, Artificial sequence
  Synthetic oligonucleotide
ggcttgaatgtcaccc

SEQ ID NO: 2048 - DNA, 16 bp, Artificial sequence
  Synthetic oligonucleotide
aatgcttgtcaaaagg

SEQ ID NO: 2049 - DNA, 16 bp, Artificial sequence
  Synthetic oligonucleotide
ctttaccaagaccgct

SEQ ID NO: 2050 - DNA, 16 bp, Artificial sequence
  Synthetic oligonucleotide
taagtgttagaactaa

SEQ ID NO: 2051 - DNA, 16 bp, Artificial sequence
  Synthetic oligonucleotide
accaacttttagtatt

SEQ ID NO: 2052 - DNA, 16 bp, Artificial sequence
  Synthetic oligonucleotide
catcgacacagcatca

SEQ ID NO: 2053 - DNA, 16 bp, Artificial sequence
  Synthetic oligonucleotide
ccaaagtcttaatgtg

SEQ ID NO: 2054 - DNA, 16 bp, Artificial sequence
  Synthetic oligonucleotide
ccatctcttagtgtcc

SEQ ID NO: 2055 - DNA, 16 bp, Artificial sequence
  Synthetic oligonucleotide
cttaggaatattgcca

SEQ ID NO: 2056 - DNA, 16 bp, Artificial sequence
  Synthetic oligonucleotide
agggttagtgttggtt

SEQ ID NO: 2057 - DNA, 16 bp, Artificial sequence
  Synthetic oligonucleotide
tctgtttgacttagtc

SEQ ID NO: 2058 - DNA, 16 bp, Artificial sequence
  Synthetic oligonucleotide
cttagggcacctcaag

SEQ ID NO: 2059 - DNA, 16 bp, Artificial sequence
  Synthetic oligonucleotide
taatctggtcatatgg

SEQ ID NO: 2060 - DNA, 16 bp, Artificial sequence
  Synthetic oligonucleotide
tgcttacggagcatag

SEQ ID NO: 2061 - DNA, 16 bp, Artificial sequence
  Synthetic oligonucleotide
ctctagacgggaagct

SEQ ID NO: 2062 - DNA, 16 bp, Artificial sequence
  Synthetic oligonucleotide
gatcatcattattacc

SEQ ID NO: 2063 - DNA, 16 bp, Artificial sequence
  Synthetic oligonucleotide
ttgcagtgccctggcc

SEQ ID NO: 2064 - DNA, 16 bp, Artificial sequence
  Synthetic oligonucleotide
attcagaccagggtaa

SEQ ID NO: 2065 - DNA, 16 bp, Artificial sequence
  Synthetic oligonucleotide
atgcctgtattagctc

SEQ ID NO: 2066 - DNA, 16 bp, Artificial sequence
  Synthetic oligonucleotide
agcctctttgtagcag

SEQ ID NO: 2067 - DNA, 16 bp, Artificial sequence
  Synthetic oligonucleotide
gaggttattttaacag

SEQ ID NO: 2068 - DNA, 16 bp, Artificial sequence
  Synthetic oligonucleotide
atactgagaggaaatg

SEQ ID NO: 2069 - DNA, 16 bp, Artificial sequence
  Synthetic oligonucleotide
atgacatttcagagta

SEQ ID NO: 2070 - DNA, 16 bp, Artificial sequence
  Synthetic oligonucleotide
gtgtaaagatgtgagt

SEQ ID NO: 2071 - DNA, 16 bp, Artificial sequence
  Synthetic oligonucleotide
tgctgcactcaaagag

SEQ ID NO: 2072 - DNA, 16 bp, Artificial sequence
  Synthetic oligonucleotide
tcacaagagactggac

SEQ ID NO: 2073 - DNA, 16 bp, Artificial sequence
  Synthetic oligonucleotide
tggtggacattggccg

SEQ ID NO: 2074 - DNA, 16 bp, Artificial sequence
  Synthetic oligonucleotide
ggtgatggttgttttg

SEQ ID NO: 2075 - DNA, 16 bp, Artificial sequence
  Synthetic oligonucleotide
gaatgcatccaaatat

SEQ ID NO: 2076 - DNA, 16 bp, Artificial sequence
  Synthetic oligonucleotide
gtggtctagcagctca

SEQ ID NO: 2077 - DNA, 16 bp, Artificial sequence
  Synthetic oligonucleotide
cattacataagacatt

SEQ ID NO: 2078 - DNA, 16 bp, Artificial sequence
  Synthetic oligonucleotide
cctcaagtgactcaca

SEQ ID NO: 2079 - DNA, 16 bp, Artificial sequence
  Synthetic oligonucleotide
ctttagcacctctgaa

SEQ ID NO: 2080 - DNA, 16 bp, Artificial sequence
  Synthetic oligonucleotide
atttcccaaccagctg

SEQ ID NO: 2081 - DNA, 16 bp, Artificial sequence
  Synthetic oligonucleotide
ttatcatctttgcaga

SEQ ID NO: 2082 - DNA, 16 bp, Artificial sequence
  Synthetic oligonucleotide
ttttagttaggtgaaa

SEQ ID NO: 2083 - DNA, 16 bp, Artificial sequence
  Synthetic oligonucleotide
ctcactgattcacata

SEQ ID NO: 2084 - DNA, 16 bp, Artificial sequence
  Synthetic oligonucleotide
tgagaaacaaaccctc

SEQ ID NO: 2085 - DNA, 16 bp, Artificial sequence
  Synthetic oligonucleotide
tgtgcaaatgatcatg

SEQ ID NO: 2086 - DNA, 16 bp, Artificial sequence
  Synthetic oligonucleotide
taggctcctgggacct

SEQ ID NO: 2087 - DNA, 16 bp, Artificial sequence
  Synthetic oligonucleotide
atcttgggatgcacag

SEQ ID NO: 2088 - DNA, 16 bp, Artificial sequence
  Synthetic oligonucleotide
tcatggcttccagtgt

SEQ ID NO: 2089 - DNA, 16 bp, Artificial sequence
  Synthetic oligonucleotide
ttcaatgtggcttcta

SEQ ID NO: 2090 - DNA, 16 bp, Artificial sequence
  Synthetic oligonucleotide
ttaattctacctgtgt

SEQ ID NO: 2091 - DNA, 16 bp, Artificial sequence
  Synthetic oligonucleotide
tgaaatatctcattag

SEQ ID NO: 2092 - DNA, 16 bp, Artificial sequence
  Synthetic oligonucleotide
tgtctactatgggagc

SEQ ID NO: 2093 - DNA, 16 bp, Artificial sequence
  Synthetic oligonucleotide
atggtaatacttttgg

SEQ ID NO: 2094 - DNA, 16 bp, Artificial sequence
  Synthetic oligonucleotide
actggtagctcctggc

SEQ ID NO: 2095 - DNA, 16 bp, Artificial sequence
  Synthetic oligonucleotide
tgattgtgcagacagt

SEQ ID NO: 2096 - DNA, 16 bp, Artificial sequence
  Synthetic oligonucleotide
tatttccgttaaccat

SEQ ID NO: 2097 - DNA, 16 bp, Artificial sequence
  Synthetic oligonucleotide
ctgagttttaggtctg

SEQ ID NO: 2098 - DNA, 16 bp, Artificial sequence
  Synthetic oligonucleotide
gaatcatcttagtggc

SEQ ID NO: 2099 - DNA, 16 bp, Artificial sequence
  Synthetic oligonucleotide
ttgttcttgaccgtgt

SEQ ID NO: 2100 - DNA, 16 bp, Artificial sequence
  Synthetic oligonucleotide
gtggtaaggcatacta

SEQ ID NO: 2101 - DNA, 16 bp, Artificial sequence
  Synthetic oligonucleotide
ggtgtacatggtctgc

SEQ ID NO: 2102 - DNA, 16 bp, Artificial sequence
  Synthetic oligonucleotide
gcattgcattgcatag

SEQ ID NO: 2103 - DNA, 16 bp, Artificial sequence
  Synthetic oligonucleotide
ggaaaaccaatcctgt

SEQ ID NO: 2104 - DNA, 16 bp, Artificial sequence
  Synthetic oligonucleotide
agctgtctcctctact

SEQ ID NO: 2105 - DNA, 16 bp, Artificial sequence
  Synthetic oligonucleotide
caaactttgcagccta

SEQ ID NO: 2106 - DNA, 16 bp, Artificial sequence
  Synthetic oligonucleotide
gagactttgcaggcac

SEQ ID NO: 2107 - DNA, 16 bp, Artificial sequence
  Synthetic oligonucleotide
tcggcttgaatgtcac

SEQ ID NO: 2108 - DNA, 16 bp, Artificial sequence
  Synthetic oligonucleotide
gtaaatgcttgtcaaa

SEQ ID NO: 2109 - DNA, 16 bp, Artificial sequence
  Synthetic oligonucleotide
agtctttaccaagacc

SEQ ID NO: 2110 - DNA, 16 bp, Artificial sequence
  Synthetic oligonucleotide
catgacatcccagttc

SEQ ID NO: 2111 - DNA, 16 bp, Artificial sequence
  Synthetic oligonucleotide
aaccaacttttagtat

SEQ ID NO: 2112 - DNA, 16 bp, Artificial sequence
  Synthetic oligonucleotide
ccatcgacacagcatc

SEQ ID NO: 2113 - DNA, 16 bp, Artificial sequence
  Synthetic oligonucleotide
ctacttatccccaaag

SEQ ID NO: 2114 - DNA, 16 bp, Artificial sequence
  Synthetic oligonucleotide
gccatctcttagtgtc

SEQ ID NO: 2115 - DNA, 16 bp, Artificial sequence
  Synthetic oligonucleotide
ccttaggaatattgcc

SEQ ID NO: 2116 - DNA, 16 bp, Artificial sequence
  Synthetic oligonucleotide
gagggttagtgttggt

SEQ ID NO: 2117 - DNA, 16 bp, Artificial sequence
  Synthetic oligonucleotide
ctggtttgtgggttct

SEQ ID NO: 2118 - DNA, 16 bp, Artificial sequence
  Synthetic oligonucleotide
tcttagggcacctcaa

SEQ ID NO: 2119 - DNA, 16 bp, Artificial sequence
  Synthetic oligonucleotide
ttaatctggtcatatg

SEQ ID NO: 2120 - DNA, 16 bp, Artificial sequence
  Synthetic oligonucleotide
acaaaagcgacaaggt

SEQ ID NO: 2121 - DNA, 16 bp, Artificial sequence
  Synthetic oligonucleotide
ttgagtctcctgacca

SEQ ID NO: 2122 - DNA, 16 bp, Artificial sequence
  Synthetic oligonucleotide
cgatcatcattattac

SEQ ID NO: 2123 - DNA, 16 bp, Artificial sequence
  Synthetic oligonucleotide
attgcagtgccctggc

SEQ ID NO: 2124 - DNA, 16 bp, Artificial sequence
  Synthetic oligonucleotide
tattcagaccagggta

SEQ ID NO: 2125 - DNA, 16 bp, Artificial sequence
  Synthetic oligonucleotide
gatgcctgtattagct

SEQ ID NO: 2126 - DNA, 16 bp, Artificial sequence
  Synthetic oligonucleotide
gcagcctctttgtagc

SEQ ID NO: 2127 - DNA, 16 bp, Artificial sequence
  Synthetic oligonucleotide
ctgaggttattttaac

SEQ ID NO: 2128 - DNA, 16 bp, Artificial sequence
  Synthetic oligonucleotide
tatactgagaggaaat

SEQ ID NO: 2129 - DNA, 16 bp, Artificial sequence
  Synthetic oligonucleotide
atatgacatttcagag

SEQ ID NO: 2130 - DNA, 16 bp, Artificial sequence
  Synthetic oligonucleotide
cgtgtaaagatgtgag

SEQ ID NO: 2131 - DNA, 16 bp, Artificial sequence
  Synthetic oligonucleotide
aatgctgcactcaaag

SEQ ID NO: 2132 - DNA, 16 bp, Artificial sequence
  Synthetic oligonucleotide
taacaaactatgccta

SEQ ID NO: 2133 - DNA, 16 bp, Artificial sequence
  Synthetic oligonucleotide
aggaacttgcttaagt

SEQ ID NO: 2134 - DNA, 16 bp, Artificial sequence
  Synthetic oligonucleotide
agcagctcatctccct

SEQ ID NO: 2135 - DNA, 16 bp, Artificial sequence
  Synthetic oligonucleotide
agttaggtgaaaaagg

SEQ ID NO: 2136 - DNA, 16 bp, Artificial sequence
  Synthetic oligonucleotide
actgattcacataata

SEQ ID NO: 2137 - DNA, 16 bp, Artificial sequence
  Synthetic oligonucleotide
ctatgggagccacatg

SEQ ID NO: 2138 - DNA, 16 bp, Artificial sequence
  Synthetic oligonucleotide
cagacagtaactggta

SEQ ID NO: 2139 - DNA, 16 bp, Artificial sequence
  Synthetic oligonucleotide
ttccgttaaccatcaa

SEQ ID NO: 2140 - DNA, 16 bp, Artificial sequence
  Synthetic oligonucleotide
gttaaactaccgaacg

SEQ ID NO: 2141 - DNA, 16 bp, Artificial sequence
  Synthetic oligonucleotide
ctatggtagagtggta

SEQ ID NO: 2142 - DNA, 16 bp, Artificial sequence
  Synthetic oligonucleotide
ggacaataagagctgt

SEQ ID NO: 2143 - DNA, 16 bp, Artificial sequence
  Synthetic oligonucleotide
agcatcattggaagac

SEQ ID NO: 2144 - DNA, 16 bp, Artificial sequence
  Synthetic oligonucleotide
gtgttagaactaaggc

SEQ ID NO: 2145 - DNA, 16 bp, Artificial sequence
  Synthetic oligonucleotide
tttctgagcagataaa

SEQ ID NO: 2146 - DNA, 16 bp, Artificial sequence
  Synthetic oligonucleotide
taggaatattgccagg

SEQ ID NO: 2147 - DNA, 16 bp, Artificial sequence
  Synthetic oligonucleotide
aactctgtagctggtt

SEQ ID NO: 2148 - DNA, 16 bp, Artificial sequence
  Synthetic oligonucleotide
tcttgatagtgaatgt

SEQ ID NO: 2149 - DNA, 16 bp, Artificial sequence
  Synthetic oligonucleotide
ccaatcagaggaagcc

SEQ ID NO: 2150 - DNA, 16 bp, Artificial sequence
  Synthetic oligonucleotide
ctgtctctaattttac

SEQ ID NO: 2151 - DNA, 16 bp, Artificial sequence
  Synthetic oligonucleotide
gacatttcagagtata

SEQ ID NO: 2152 - DNA, 16 bp, Artificial sequence
  Synthetic oligonucleotide
gcaaatcggatctttg

SEQ ID NO: 2153 - DNA, 16 bp, Artificial sequence
  Synthetic oligonucleotide
agaaggtagaggttcc

SEQ ID NO: 2154 - DNA, 16 bp, Artificial sequence
  Synthetic oligonucleotide
cagggcaggatgctga

SEQ ID NO: 2155 - DNA, 16 bp, Artificial sequence
  Synthetic oligonucleotide
tgactcacagactctt

SEQ ID NO: 2156 - DNA, 16 bp, Artificial sequence
  Synthetic oligonucleotide
agaaacaaaccctccg

SEQ ID NO: 2157 - DNA, 16 bp, Artificial sequence
  Synthetic oligonucleotide
actatgggagccacat

SEQ ID NO: 2158 - DNA, 16 bp, Artificial sequence
  Synthetic oligonucleotide
ttgtgcagacagtaac

SEQ ID NO: 2159 - DNA, 16 bp, Artificial sequence
  Synthetic oligonucleotide
ttttaggtctgggtat

SEQ ID NO: 2160 - DNA, 16 bp, Artificial sequence
  Synthetic oligonucleotide
attgcattgcatagcc

SEQ ID NO: 2161 - DNA, 16 bp, Artificial sequence
  Synthetic oligonucleotide
ctttgcagcctatccc

SEQ ID NO: 2162 - DNA, 16 bp, Artificial sequence
  Synthetic oligonucleotide
tcagcatcattggaag

SEQ ID NO: 2163 - DNA, 16 bp, Artificial sequence
  Synthetic oligonucleotide
agtgttagaactaagg

SEQ ID NO: 2164 - DNA, 16 bp, Artificial sequence
  Synthetic oligonucleotide
caaagtcttaatgtgg

SEQ ID NO: 2165 - DNA, 16 bp, Artificial sequence
  Synthetic oligonucleotide
tgtttgacttagtccg

SEQ ID NO: 2166 - DNA, 16 bp, Artificial sequence
  Synthetic oligonucleotide
aatctggtcatatggt

SEQ ID NO: 2167 - DNA, 16 bp, Artificial sequence
  Synthetic oligonucleotide
tgcagtgccctggcct

SEQ ID NO: 2168 - DNA, 16 bp, Artificial sequence
  Synthetic oligonucleotide
aggttattttaacagc

SEQ ID NO: 2169 - DNA, 16 bp, Artificial sequence
  Synthetic oligonucleotide
tgacatttcagagtat

SEQ ID NO: 2170 - DNA, 16 bp, Artificial sequence
  Synthetic oligonucleotide
tcaatgtggcttctag

SEQ ID NO: 2171 - DNA, 16 bp, Artificial sequence
  Synthetic oligonucleotide
attcaatgtggcttct

SEQ ID NO: 2172 - DNA, 16 bp, Artificial sequence
  Synthetic oligonucleotide
tattcaatgtggcttc

SEQ ID NO: 2173 - DNA, 21 bp, Artificial Sequence
  Description of Artificial Sequence: Synthetic oligonucleotide
  Description of Combined DNA/RNA Molecule: Synthetic oligonucleotide
gguccucucagaucuugugtt

SEQ ID NO: 2174 - DNA, 21 bp, Artificial Sequence
  Description of Artificial Sequence: Synthetic oligonucleotide
  Description of Combined DNA/RNA Molecule: Synthetic oligonucleotide
ggagugagugacaacguactt

SEQ ID NO: 2175 - DNA, 21 bp, Artificial Sequence
  Description of Artificial Sequence: Synthetic oligonucleotide
  Description of Combined DNA/RNA Molecule: Synthetic oligonucleotide
gguucuuggaagagaagggtt

SEQ ID NO: 2176 - DNA, 16 bp, Artificial Sequence
  Description of Artificial Sequence: Synthetic oligonucleotide
ggccaatacgccgtca

SEQ ID NO: 2177 - DNA, 16 bp, Artificial Sequence
  Description of Artificial Sequence: Synthetic oligonucleotide
tatttttggtgtatcc

SEQ ID NO: 2178 - DNA, 16 bp, Artificial Sequence
  Description of Artificial Sequence: Synthetic oligonucleotide
ggccaatacgccgtca

(19) United States
(12) Patent Application Publication
(10) Pub. No.: US 2026/0234631 A1
(43) Pub. Date: Aug. 13, 2026

(54) MODULATORS OF PNPLA3 EXPRESSION

(71) Applicant: AstraZeneca AB, Södertälje (SE)

(72) Inventor: Daniel LINDEN, Södertälje (SE)

(21) Appl. No.: 17/754,844

(22) PCT Filed: Oct. 12, 2020
(86) PCT No.: PCT/IB2020/059566

§ 371 Date: Apr. 13, 2022

Related U.S. Application Data
(60) Provisional application No. 62/914,660, filed on Oct. 14, 2019.

(51) Int. Cl. C12N 15/113; A61P 1/16

(52) U.S. Cl. CPC C12N 15/1137; A61P 1/16; C12N 2310/11; C12N 2310/14

(57) ABSTRACT

The present embodiments provide methods, compounds, and compositions useful for inhibiting PNPLA3 expression, which may be useful for treating, preventing, or ameliorating a disease associated with PNPLA3. In some embodiments, the methods, compounds, and compositions are useful for treating, preventing, or ameliorating a disease associated with PNPLA3 having an I148M mutation.

### SEQUENCE LISTING

The present application is being filed along with a Sequence Listing in electronic format. The Sequence Listing is provided as a file entitled BIOL0317USLSEQ_ST25.txt created Sep. 13, 2018, which is 480 kb in size. The information in the electronic format of the sequence listing is incorporated herein by reference in its entirety.

### FIELD

The present embodiments provide methods, compounds, and compositions useful for inhibiting PNPLA3 (patatin like phospholipase domain containing 3; hypothetical protein dJ796I17.1; adiponutrin; DJ796I17.1) expression, and in certain instances, reducing the amount of PNPLA3 protein in a cell or animal, which can be useful for treating, preventing, or ameliorating a disease associated with PNPLA3. In some embodiments, the methods, compounds, and compositions are useful for treating, preventing, or ameliorating a disease associated with PNPLA3 having an I148M mutation.

### Background

Non-alcoholic fatty liver disease (NAFLD) covers a spectrum of liver disease from steatosis to nonalcoholic steatohepatitis (NASH) and cirrhosis. NAFLD is defined as fat accumulation in the liver exceeding 5% by weight, in the absence of significant alcohol consumption, steatogenic medication, or hereditary disorders (Kotronen et al, Arterioscler Thromb. Vasc. Biol. 2008, 28: 27-38).

Non-alcoholic steatohepatitis (NASH) is an aggressive variant of NAFLD with signs of inflammation and hepatic injury. NASH is defined histologically by macrovesicular steatosis, hepatocellular ballooning, and lobular inflammatory infiltrates (Sanyal, Hepatol. Res. 2011. 41: 670-4). NASH is estimated to affect 2-3% of the general population. In the presence of other pathologies, such as obesity or diabetes, the estimated prevalence increases to 7% and 62% respectively (Hashimoto et al, J. Gastroenterol. 2011. 46(1): 63-69).

PNPLA3 is a 481 amino acid member of the patatin-like phospholipase domain-containing family that is expressed in the ER and on lipid droplets. In humans, PNPLA3 is highly expressed in the liver, whereas adipose tissue expression is five-fold less (Huang et al, Proc. Natl. Acad. Sci. USA 2010. 107: 7892-7).

### SUMMARY

Certain embodiments provided herein are compounds and methods for reducing the amount or activity of PNPLA3 mRNA, and in certain embodiments, reducing the amount of PNPLA3 protein in a cell or animal. In certain embodiments, the animal has a liver disease. In certain embodiments, the disease is NASH. In certain embodiments, the disease is NAFLD. In certain embodiments, the disease is hepatic steatosis. In certain embodiments, the disease is liver cirrhosis. In certain embodiments, the disease is hepatocellular carcinoma. In certain embodiments, the disease is alcoholic liver disease. In certain embodiments, the disease is alcoholic steatohepatitis (ASH). In certain embodiments, the disease is HCV hepatitis. In certain embodiments, the disease is chronic hepatitis. In certain embodiments, the disease is hereditary hemochromatosis. In certain embodiments, the disease is primary sclerosing cholangitis. Certain compounds provided herein are directed to compounds and compositions that reduce liver damage, steatosis, liver fibrosis, liver inflammation, liver scarring or cirrhosis, liver failure, liver enlargement, elevated transaminases, or hepatic fat accumulation in an animal.

Certain embodiments provided herein are directed to potent and tolerable compounds and compositions useful for inhibiting PNPLA3 expression, which can be useful for treating, preventing, ameliorating, or slowing progression of liver diseases. Certain embodiments provided herein are directed to compounds and compositions that are more potent or have greater therapeutic value than compounds publicly disclosed.

In some embodiments, the present disclosure provides a method of treating an individual having or at risk of having liver disease, comprising administering a compound targeted to PNPLA3 to the individual, wherein the individual has an I148M mutation in patatin-like phospholipase domain-containing protein 3 (PNPLA3).

In some embodiments, the present disclosure provides a method of reducing one or more of liver damage, hepatic steatosis, liver inflammation, liver fibrosis, and hepatic lipogenesis in an individual, comprising administering a compound targeted to PNPLA3 to the individual, wherein the individual has an I148M mutation in patatin-like phospholipase domain-containing protein 3 (PNPLA3).

In some embodiments, the present disclosure provides a method of reducing protein levels of one or more of haptoglobin, MCP1, and TIMP2 in an individual, comprising administering a compound targeted to PNPLA3 to the individual, wherein the individual has an I148M mutation in patatin-like phospholipase domain-containing protein 3 (PNPLA3).

In some embodiments, the liver disease is selected from non-alcoholic fatty liver disease (NAFLD), hepatic steatosis, non-alcoholic steatohepatitis (NASH), liver cirrhosis, hepatocellular carcinoma, alcoholic liver disease, alcoholic steatohepatitis (ASH), HCV hepatitis, chronic hepatitis, hereditary hemochromatosis, or primary sclerosing cholangitis. In some embodiments, the liver disease is hepatic steatosis.

In some embodiments, the method reduces or inhibits liver inflammation or liver fibrosis. In some embodiments, reducing or inhibiting liver inflammation comprises reducing liver macrophage levels. In some embodiments, the liver macrophage levels are reduced by at least 20% relative to an individual not administered the compound targeted to PNPLA3, as measured by immunohistochemical staining of a liver section of the individual.

In some embodiments, the protein levels of haptoglobin are reduced by at least 20% relative to an individual not administered the compound targeted to PNPLA3, as measured by a colorimetric assay of serum or plasma of the individual. In some embodiments, the protein levels of MCP1 are reduced by at least 20% relative to an individual not administered the compound targeted to PNPLA3, as measured by immunoblotting of a liver sample of the individual. In some embodiments, the protein levels of TIMP2 are reduced by at least 20% relative to an individual not administered the compound targeted to PNPLA3, as measured by immunoblotting of a liver sample of the individual.

In some embodiments, the individual has a homozygous I148M mutation in PNPLA3. In some embodiments, the individual is a human individual.

In some embodiments, the compound targeted to PNPLA3, in an individual having an I148M mutation, is an antisense compound targeted to PNPLA3. In some embodiments, the antisense compound targeted to PNPLA3 is a short interfering RNA (siRNA). In some embodiments, the antisense compound targeted to PNPLA3 is an antisense oligonucleotide (ASO).

In some embodiments, the compound targeted to PNPLA3, in an individual having an I148M mutation, comprises a modified oligonucleotide 8 to 80 linked nucleosides in length having a nucleobase sequence comprising at least 8, at least 9, at least 10, at least 11, or at least 12 contiguous nucleobases of any of the nucleobase sequences of SEQ ID NOs: 17-2169. In some embodiments, the compound targeted to PNPLA3 comprises a modified oligonucleotide 8 to 80 nucleosides in length and having a nucleobase sequence comprising the nucleobase sequence of any one of SEQ ID NOs: 17-2169. In some embodiments, the compound targeted to PNPLA3 comprises a modified oligonucleotide having a nucleobase sequence consisting of any one of SEQ ID NOs: 17-2169.

In some embodiments, the compound targeted to PNPLA3, in an individual having an I148M mutation, comprises a modified oligonucleotide 8 to 80 linked nucleosides in length, wherein the modified oligonucleotide has a nucleobase sequence comprising a portion of at least 8 contiguous nucleobases 100% complementary to an equal length portion of nucleobases 5567-5642, 5644-5731, 5567-5731, 5567-5620, 13697-13733, 20553-20676, 20664-20824, 20553-20824, or 25844-25912 of SEQ ID NO: 2, and wherein the nucleobase sequence of the modified oligonucleotide is at least 85%, at least 90%, at least 95%, or 100% complementary to SEQ ID NO: 2. In some embodiments, the compound targeted to PNPLA3, in an individual having an I148M mutation, comprises a modified oligonucleotide 8 to 80 linked nucleosides in length, wherein the modified oligonucleotide has a nucleobase sequence complementary within nucleobases 5567-5642, 5644-5731, 5567-5731, 5567-5620, 13697-13733, 20553-20676, 20664-20824, 20553-20824, or 25844-25912 of SEQ ID NO: 2, and wherein said modified oligonucleotide is at least 85%, at least 90%, at least 95%, or 100% complementary to SEQ ID NO: 2.

In some embodiments, the compound targeted to PNPLA3, in an individual having an I148M mutation, comprises a modified oligonucleotide 8 to 80 linked nucleosides in length, wherein the modified oligonucleotide has a nucleobase sequence comprising a portion of at least 8 contiguous nucleobases complementary to an equal length portion of nucleobases 5567-5642, 5644-5731, 5567-5731, 5567-5620, 13697-13733, 20553-20676, 20664-20824, 20553-20824, or 25844-25912 of a PNPLA3 nucleic acid having the nucleobase sequence of SEQ ID NO: 2, wherein the nucleobase sequence of the modified oligonucleotide is complementary to SEQ ID NO: 2. In some embodiments, the compound targeted to PNPLA3, in an individual having an I148M mutation, comprises a modified oligonucleotide 8 to 80 linked nucleosides in length, wherein the modified oligonucleotide has a nucleobase sequence comprising a 16 nucleobase portion complementary to an equal length portion of nucleobases 5567-5642, 5644-5731, 5567-5731, 5567-5620, 13697-13733, 20553-20676, 20664-20824, 20553-20824, or 25844-25912 of SEQ ID NO: 2.

In some embodiments, the compound targeted to PNPLA3, in an individual having an I148M mutation, comprises a modified oligonucleotide 8 to 80 linked nucleosides in length, wherein the modified oligonucleotide has a nucleobase sequence comprising any one of SEQ ID NOs: 1089, 1757, 141, 1982, 330, 1665, 408, 830, and 899. In some embodiments, the compound targeted to PNPLA3, in an individual having an I148M mutation, comprises a modified oligonucleotide having a nucleobase sequence consisting of any one of SEQ ID NOs: 1089, 1757, 141, 1982, 330, 1665, 408, 830, and 899.

In some embodiments, the modified oligonucleotide has a nucleobase sequence at least 80%, at least 85%, at least 90%, at least 95% or 100% complementary to SEQ ID NO: 2 over the entire length of the nucleobase sequence.

In some embodiments, the modified nucleotide comprises at least one modification selected from at least one modified internucleoside linkage, at least one modified sugar, and at least one modified nucleobase. In some embodiments, the modified internucleoside linkage is a phosphorothioate internucleoside linkage.

In some embodiments, the modified sugar is a bicyclic sugar. In some embodiments, the bicyclic sugar is selected from the group consisting of: 4′—(CH2)—O—2′ (LNA); 4′—(CH2)2—O—2′ (ENA); and 4′—CH(CH3)—O—2′ (cEt). In some embodiments, the modified sugar is 2′-O-methoxyethyl. In some embodiments, the modified nucleobase is a 5-methylcytosine.

In some embodiments, the modified oligonucleotide comprises: a gap segment consisting of linked deoxynucleosides; a 5′ wing segment consisting of linked nucleosides; and a 3′ wing segment consisting of linked nucleosides; wherein the gap segment is positioned immediately adjacent to and between the 5′ wing segment and the 3′ wing segment and wherein each nucleoside of each wing segment comprises a modified sugar.

In some embodiments, the compound targeted to PNPLA3, in an individual having an I148M mutation, is single-stranded. In some embodiments, the compound targeted to PNPLA3 is double-stranded. In some embodiments, the compound targeted to PNPLA3, in an individual having an I148M mutation, comprises ribonucleotides. In some embodiments, the compound targeted to PNPLA3, in an individual having an I148M mutation, comprises deoxyribonucleotides.

In some embodiments, the modified oligonucleotide consists of 10 to 30 linked nucleosides. In some embodiments, the modified oligonucleotide consists of 12 to 30 linked nucleosides. In some embodiments, the modified oligonucleotide consists of 15 to 30 linked nucleosides.

In some embodiments, the compound targeted to PNPLA3, in an individual having an I148M mutation, comprises a modified oligonucleotide 16 linked nucleosides in length, wherein the modified oligonucleotide has a nucleobase sequence comprising any one of SEQ ID NOs: 1089, 1757, 141, 1982, 330, 1665, 408, 830, and 899, wherein the modified oligonucleotide comprises: a gap segment consisting of linked deoxynucleosides; a 5′ wing segment consisting of linked nucleosides; and a 3′ wing segment consisting of linked nucleosides; wherein the gap segment is positioned between the 5′ wing segment and the 3′ wing segment and wherein each nucleoside of each wing segment comprises a modified sugar.

In some embodiments, the compound targeted to PNPLA3, in an individual having an I148M mutation, comprises a modified oligonucleotide 16 linked nucleosides in length, wherein the modified oligonucleotide has a nucleobase sequence comprising any one of SEQ ID NOs: 1089, 1757, 141, 1982, 330, 1665, 408, 830, and 899, wherein the modified oligonucleotide comprises: a gap segment consisting of ten linked deoxynucleosides; a 5′ wing segment consisting of three linked nucleosides; and a 3′ wing segment consisting of three linked nucleosides; wherein the gap segment is positioned between the 5′ wing segment and the 3′ wing segment; wherein the 5′ wing segment and the 3′ wing segment comprise cEt sugars; wherein each internucleoside linkage is a phosphorothioate linkage; and wherein each cytosine is a 5-methylcytosine.

In some embodiments, the compound targeted to PNPLA3, in an individual having an I148M mutation, comprises a conjugated moiety and a conjugate linker. In some embodiments, the conjugate group comprises a GalNAc cluster comprising 1 to 3 GalNAc ligands.

In some embodiments, the conjugate linker consists of a single bond. In some embodiments, the conjugate linker is cleavable. In some embodiments, the conjugate linker comprises 1 to 3 linker-nucleosides. In some embodiments, the conjugate group is attached to the modified oligonucleotide at the 5′-end of the modified oligonucleotide. In some embodiments, the conjugate group is attached to the modified oligonucleotide at the 3′-end of the modified oligonucleotide.

In some embodiments, the compound targeted to PNPLA3, in an individual having an I148M mutation, comprises the following formula or salt thereof:

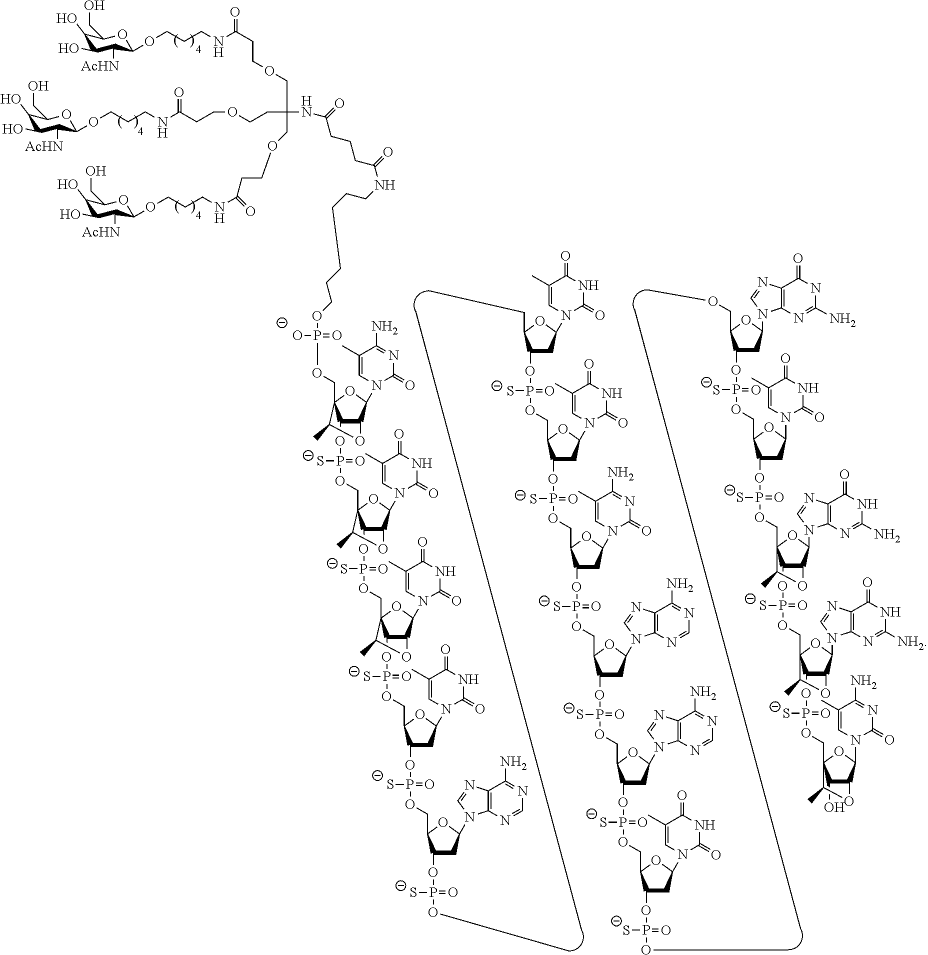

In some embodiments, the compound targeted to PNPLA3, in an individual having an I148M mutation, comprises a modified oligonucleotide and a conjugate group, wherein the modified oligonucleotide is 16 linked nucleosides in length and consists of the sequence of SEQ ID NO: 1089, wherein the modified oligonucleotide comprises: a gap segment consisting of ten linked deoxynucleosides; a 5′ wing segment consisting of three linked nucleosides; and a 3′ wing segment consisting of three linked nucleosides; wherein the gap segment is positioned between the 5′ wing segment and the 3′ wing segment, wherein each nucleoside of each wing segment comprises a cEt sugar; wherein each internucleoside linkage is a phosphorothioate linkage; wherein each cytosine is a 5-methylcytosine; and wherein the conjugate group is positioned at the 5′end of the modified oligonucleotide and is

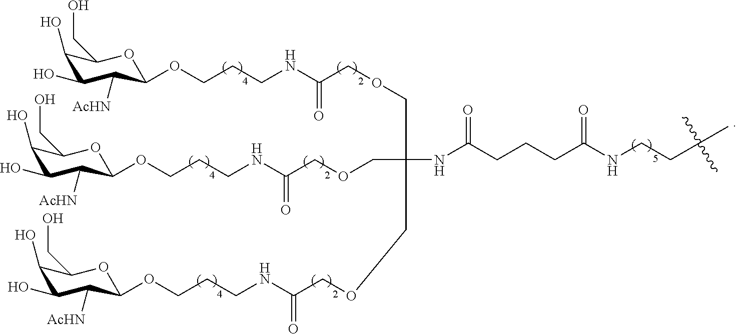

In some embodiments, the compound targeted to PNPLA3, in an individual having an I148M mutation, has the following formula or salt thereof:

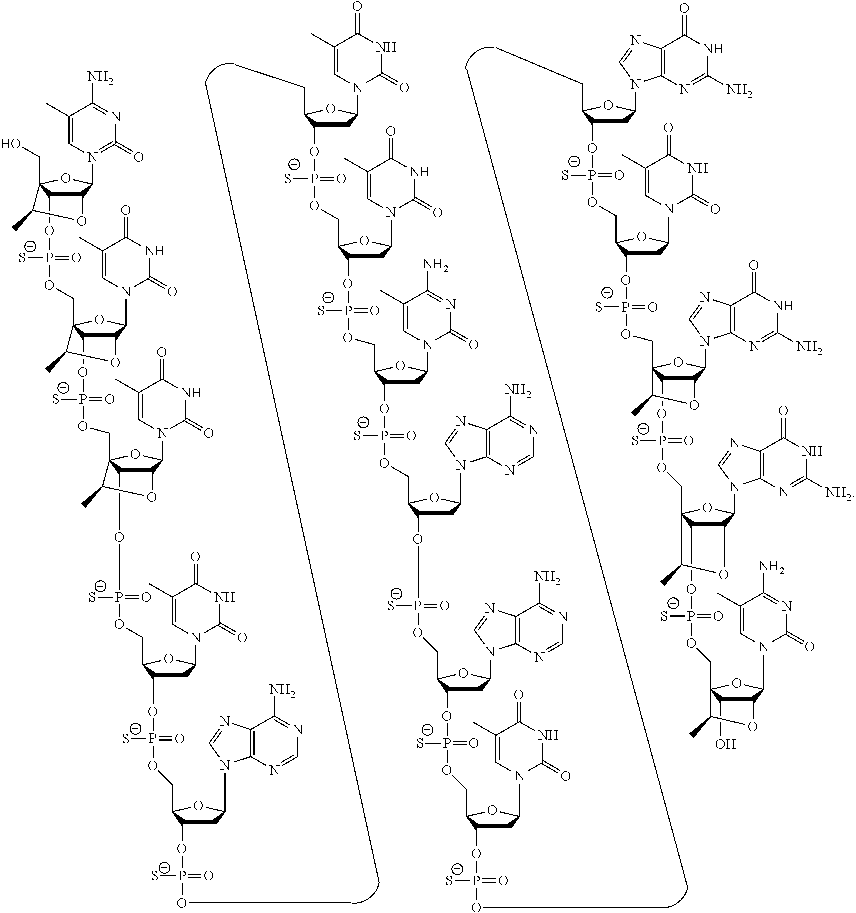

In some embodiments, the compound targeted to PNPLA3, in an individual having an I148M mutation, is a modified oligonucleotide in a pharmaceutically acceptable salt form. In some embodiments, the pharmaceutically acceptable salt is a sodium salt. In some embodiments, the pharmaceutically acceptable salt is a potassium salt.

In some embodiments, the compound targeted to PNPLA3, in an individual having an I148M mutation, is administered to the individual as a composition comprising the compound targeted to PNPLA3 and a pharmaceutically acceptable carrier. In some embodiments, the compound targeted to PNPLA3, in an individual having an I148M mutation, is administered parenterally to the individual.

In some embodiments, the compound targeted to PNPLA3, in an individual having an I148M mutation, comprises a modified oligonucleotide, wherein the modified oligonucleotide has a nucleobase sequence at least 90% identical to SEQ ID NO: 115. In some embodiments, the compound targeted to PNPLA3, in an individual having an I148M mutation, comprises a modified oligonucleotide, wherein the modified oligonucleotide has a nucleobase sequence at least 90% identical to any one of SEQ ID NOs: 2170-2172.

In some embodiments, the compound targeted to PNPLA3, in an individual having an I148M mutation, consists of the sequence of SEQ ID NO: 115, wherein the modified oligonucleotide comprises: a gap segment consisting of ten linked deoxynucleosides; a 5′ wing segment consisting of three linked nucleosides; and a 3′ wing segment consisting of three linked nucleosides; wherein the gap segment is positioned between the 5′ wing segment and the 3′ wing segment, wherein each nucleoside of each wing segment comprises a cEt sugar; wherein each internucleoside linkage is a phosphorothioate linkage; and wherein each cytosine is a 5-methylcytosine. In some embodiments, the compound targeted to PNPLA3, in an individual having an I148M mutation, further comprises a conjugate group, wherein the conjugate group is positioned at the 5′end of the modified oligonucleotide and is

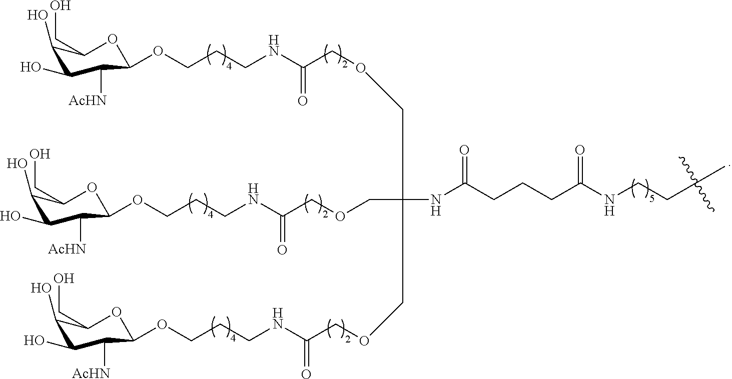

### BRIEF DESCRIPTION OF THE FIGURES

FIGS. 1A-1F relate to Example 11. The results in FIGS. 1A-1C relate to human HepG2 cells subjected to either a control ASO or PNPLA3 ASO described in embodiments herein. FIG. 1A shows PNPLA3 mRNA levels. FIG. 1B shows the Oil Red O (ORO) staining area. FIG. 1C shows an image of the ORO staining. The results in FIGS. 1D-1F relate to human HepG2 cells subjected to either a control siRNA or PNPLA3 siRNA described in embodiments herein. FIG. 1D shows PNPLA3 mRNA levels. FIG. 1E shows the Oil Red O (ORO) staining area. FIG. 1F shows an image of the ORO staining.

FIGS. 2A-2J relate to Example 13. The results in FIGS. 2A-2J relate to wild-type and PNPLA3 I148M mutant knock-in mice subjected to either a control ASO or PNPLA3 ASO described in embodiments herein.

FIG. 2A shows body weight gain before and after ASO treatment. FIG. 2B shows caloric intake before and after ASO treatment. FIG. 2C shows liver Pnpla3 mRNA levels measured by qPCR and normalized to the ribosomal protein large PO (RplpO). FIG. 2D shows levels of the Pnpla3 protein in hepatic lipid droplets as measured by Western blotting. FIGS. 2E and 2H show representative images of ORO-stained liver sections after 8 weeks of ASO treatment (black scale bar represents 100 μm). FIGS. 2F and 21 show liver lipid levels in PNPLA3 I148M mutant mice and wild-type mice, respectively, as assessed by MRI after 6 weeks of ASO treatment. FIGS. 2G and 2J show liver and plasma triglyceride levels in PNPLA3 I148M mutant mice and wild-type mice, respectively, as measured by biochemical assays.

FIGS. 3A-3F and 4A-4B relate to Example 14. The results in FIGS. 3A-3F and 4A-4B relate to wild-type and PNPLA3 I148M mutant knock-in mice subjected to either a control ASO or PNPLA3 ASO described in embodiments herein. FIG. 3A shows body weight as measured throughout the experiment. FIG. 3B shows caloric intake measured before and after ASO treatment. FIG. 3C shows liver Pnpla3 mRNA levels measured by qPCR and normalized to the ribosomal protein large PO (RplpO). FIG. 3D shows levels of the Pnpla3 protein in hepatic lipid droplets as measured by Western blotting. FIGS. 3E and 3F show levels of plasma ALT, AST, and triglycerides and liver triglyceride content in PNPLA3 1148M mutant mice and wild-type mice, respectively.

FIGS. 4A and 4B show liver steatosis score, lobular inflammation score, NAFLD activity score (NAS), and fibrosis stage in PNPLA3 I148M mutant mice and wild-type mice, respectively.

FIGS. 5A-5E and FIG. 6 relate to Example 15. The results in FIGS. 5A-3E and 6 relate to wild-type and PNPLA3 I148M mutant knock-in mice subjected to either a control ASO or PNPLA3 ASO described in embodiments herein. FIG. 5A shows representative images of Oil Red 0-stained liver sections (black scale bar represents 100 μm). FIGS. 5B and 5C show liver mRNA expression levels of Acc1 and Scd1 in PNPLA3 I148M mutant mice and wild-type mice, respectively. FIGS. 5D and 5E show liver lipid droplet fatty acid composition in PNPLA3 I148M mutant mice and wild-type mice, respectively.

FIG. 6 shows additional liver lipid droplet fatty acid composition in PNPLA3 I148M mutant mice and wild-type mice, including monounsaturated fatty acids (MUFA), polyunsaturated fatty acids (PUFA), and saturated fatty acids (SFA).

FIGS. 7A-7H, 8A-8E and 9A-9D relate to Example 16. The results in FIGS. 7A-7H, 8A-8E, and 9A-9D relate to wild-type and PNPLA3 I148M mutant knock-in mice subjected to either a control ASO or PNPLA3 ASO described in embodiments herein. FIGS. 7A and 7B show plasma haptoglobin levels and liver macrophage contents (as determined by Mac2 staining) in PNPLA3 I148M mutant mice and wild-type mice, respectively.

FIG. 7C shows representative images of Mac2-stained liver sections (black scale bar represents 100 μm). FIGS. 7D-7H show liver protein levels of Mcp1 (FIG. 7D), I11β (FIG. 7E), 116 (FIG. 7F), Tnfα (FIG. 7G), and αSma (FIG. 7H) in PNPLA3 I148M mutant mice and wild-type mice.

FIGS. 8A and 8B show liver Collal mRNA and protein (immunohistochemistry) levels in PNPLA3 T148M mutant mice and wild-type mice, respectively. FIG. 8C shows representative images of collagen immunohistochemistry in liver sections (black scale bar represents 100 μm). FIGS. 8D and 8E show liver hydroxyproline levels in in PNPLA3 I148M mutant mice and wild-type mice, respectively.

FIGS. 9A-9D show liver proteins of Timp2 (FIG. 9A), Mmp2 (FIG. 9B), Timp1 (FIG. 9C), and Tgfβr2 (FIG. 9D) measured with Western blot analyses in in PNPLA3 1148M mutant mice and wild-type mice.

### DETAILED DESCRIPTION

It is to be understood that both the foregoing general description and the following detailed description are exemplary and explanatory only and are not restrictive of the embodiments, as claimed. Herein, the use of the singular includes the plural unless specifically stated otherwise. As used herein, the use of “or” means “and/or” unless stated otherwise. Furthermore, the use of the term “including” as well as other forms, such as “includes” and “included,” is not limiting.

The section headings used herein are for organizational purposes only and are not to be construed as limiting the subject matter described. All documents, or portions of documents, cited in this application, including, but not limited to, patents, patent applications, articles, books, treatises, and GenBank and NCBI reference sequence records are hereby expressly incorporated by reference for the portions of the document discussed herein, as well as in their entirety.

It is understood that the sequence set forth in each SEQ ID NO in the examples contained herein is independent of any modification to a sugar moiety, an internucleoside linkage, or a nucleobase. As such, compounds defined by a SEQ ID NO may comprise, independently, one or more modifications to a sugar moiety, an internucleoside linkage, or a nucleobase. Compounds described by ION number indicate a combination of nucleobase sequence, chemical modification, and motif.

### Definitions

Unless otherwise indicated, the following terms have the following meanings:

“2′-deoxynucleoside” means a nucleoside comprising 2′-H(H) furanosyl sugar moiety, as found in naturally occurring deoxyribonucleic acids (DNA). In certain embodiments, a 2′-deoxynucleoside may comprise a modified nucleobase or may comprise an RNA nucleobase (uracil).

“2′-O-methoxyethyl” (also 2′-MOE) refers to a 2′-O(CH₂)₂—OCH₃) in the place of the 2′—OH group of a ribosyl ring. A 2′-O-methoxyethyl modified sugar is a modified sugar.

“2′-MOE nucleoside” (also 2′-O-methoxyethyl nucleoside) means a nucleoside comprising a 2′-MOE modified sugar moiety.

“2′-substituted nucleoside” or “2-modified nucleoside” means a nucleoside comprising a 2′-substituted or 2′-modified sugar moiety. As used herein, “2′-substituted” or “2-modified” in reference to a sugar moiety means a sugar moiety comprising at least one 2-substituent group other than H or OH.

“3′ target site” refers to the nucleotide of a target nucleic acid which is complementary to the 3′-most nucleotide of a particular compound.

“5′ target site” refers to the nucleotide of a target nucleic acid which is complementary to the 5′-most nucleotide of a particular compound.

“5-methylcytosine” means a cytosine with a methyl group attached to the 5 position.

“About” means within +10% of a value. For example, if it is stated, “the compounds affected about 70% inhibition of PNPLA3,” it is implied that PNPLA3 levels are inhibited within a range of 60% and 80%.

“Administration” or “administering” refers to routes of introducing a compound or composition provided herein to an individual to perform its intended function. An example of a route of administration that can be used includes, but is not limited to parenteral administration, such as subcutaneous, intravenous, or intramuscular injection or infusion.

“Administered concomitantly” or “co-administration” means administration of two or more compounds in any manner in which the pharmacological effects of both are manifest in the patient. Concomitant administration does not require that both compounds be administered in a single pharmaceutical composition, in the same dosage form, by the same route of administration, or at the same time. The effects of both compounds need not manifest themselves at the same time. The effects need only be overlapping for a period of time and need not be coextensive. Concomitant administration or co-administration encompasses administration in parallel or sequentially.

“Amelioration” refers to an improvement or lessening of at least one indicator, sign, or symptom of an associated disease, disorder, or condition. In certain embodiments, amelioration includes a delay or slowing in the progression or severity of one or more indicators of a condition or disease. The progression or severity of indicators may be determined by subjective or objective measures, which are known to those skilled in the art.

“Animal” refers to a human or non-human animal, including, but not limited to, mice, rats, rabbits, dogs, cats, pigs, and non-human primates, including, but not limited to, monkeys and chimpanzees.

“Antisense activity” means any detectable and/or measurable activity attributable to the hybridization of an antisense compound to its target nucleic acid. In certain embodiments, antisense activity is a decrease in the amount or expression of a target nucleic acid or protein encoded by such target nucleic acid compared to target nucleic acid levels or target protein levels in the absence of the antisense compound to the target.

“Antisense compound” means a compound comprising an oligonucleotide and optionally one or more additional features, such as a conjugate group or terminal group. Examples of antisense compounds include single-stranded and double-stranded compounds, such as, oligonucleotides, ribozymes, siRNAs, shRNAs, ssRNAs, and occupancy-based compounds.

“Antisense inhibition” means reduction of target nucleic acid levels in the presence of an antisense compound complementary to a target nucleic acid compared to target nucleic acid levels in the absence of the antisense compound.

“Antisense mechanisms” are all those mechanisms involving hybridization of a compound with target nucleic acid, wherein the outcome or effect of the hybridization is either target degradation or target occupancy with concomitant stalling of the cellular machinery involving, for example, transcription or splicing.

“Antisense oligonucleotide” means an oligonucleotide having a nucleobase sequence that is complementary to a target nucleic acid or region or segment thereof. In certain embodiments, an antisense oligonucleotide is specifically hybridizable to a target nucleic acid or region or segment thereof.

“Bicyclic nucleoside” or “BNA” means a nucleoside comprising a bicyclic sugar moiety. “Bicyclic sugar” or “bicyclic sugar moiety” means a modified sugar moiety comprising two rings, wherein the second ring is formed via a bridge connecting two of the atoms in the first ring thereby forming a bicyclic structure. In certain embodiments, the first ring of the bicyclic sugar moiety is a furanosyl moiety. In certain embodiments, the bicyclic sugar moiety does not comprise a furanosyl moiety.

“Branching group” means a group of atoms having at least 3 positions that are capable of forming covalent linkages to at least 3 groups. In certain embodiments, a branching group provides a plurality of reactive sites for connecting tethered ligands to an oligonucleotide via a conjugate linker and/or a cleavable moiety.

“Cell-targeting moiety” means a conjugate group or portion of a conjugate group that is capable of binding to a particular cell type or particular cell types.

“cEt” or “constrained ethyl” means a ribosyl bicyclic sugar moiety wherein the second ring of the bicyclic sugar is formed via a bridge connecting the 4′-carbon and the 2′-carbon, wherein the bridge has the formula: 4′—CH(CH₃)—O—2′, and wherein the methyl group of the bridge is in the S configuration.

“cEt nucleoside” means a nucleoside comprising a cEt modified sugar moiety.

“Chemical modification” in a compound describes the substitutions or changes through chemical reaction, of any of the units in the compound relative to the original state of such unit. “Modified nucleoside” means a nucleoside having, independently, a modified sugar moiety and/or modified nucleobase. “Modified oligonucleotide” means an oligonucleotide comprising at least one modified internucleoside linkage, a modified sugar, and/or a modified nucleobase.

“Chemically distinct region” refers to a region of a compound that is in some way chemically different than another region of the same compound. For example, a region having 2′-O-methoxyethyl nucleotides is chemically distinct from a region having nucleotides without 2′-O-methoxyethyl modifications.

“Chimeric antisense compounds” means antisense compounds that have at least 2 chemically distinct regions, each position having a plurality of subunits.

“Cleavable bond” means any chemical bond capable of being split. In certain embodiments, a cleavable bond is selected from among: an amide, a polyamide, an ester, an ether, one or both esters of a phosphodiester, a phosphate ester, a carbamate, a di-sulfide, or a peptide.

“Cleavable moiety” means a bond or group of atoms that is cleaved under physiological conditions, for example, inside a cell, an animal, or a human.

“Complementary” in reference to an oligonucleotide means the nucleobase sequence of such oligonucleotide or one or more regions thereof matches the nucleobase sequence of another oligonucleotide or nucleic acid or one or more regions thereof when the two nucleobase sequences are aligned in opposing directions. Nucleobase matches or complementary nucleobases, as described herein, are limited to the following pairs: adenine (A) and thymine (T), adenine (A) and uracil (U), cytosine (C) and guanine (G), and 5-methyl cytosine (^{m}C) and guanine (G) unless otherwise specified. Complementary oligonucleotides and/or nucleic acids need not have nucleobase complementarity at each nucleoside and may include one or more nucleobase mismatches. By contrast, “fully complementary” or “100% complementary” in reference to oligonucleotides means that such oligonucleotides have nucleobase matches at each nucleoside without any nucleobase mismatches.

“Conjugate group” means a group of atoms that is attached to an oligonucleotide. Conjugate groups include a conjugate moiety and a conjugate linker that attaches the conjugate moiety to the oligonucleotide.

“Conjugate linker” means a group of atoms comprising at least one bond that connects a conjugate moiety to an oligonucleotide.

“Conjugate moiety” means a group of atoms that is attached to an oligonucleotide via a conjugate linker.

“Contiguous” in the context of an oligonucleotide refers to nucleosides, nucleobases, sugar moieties, or internucleoside linkages that are immediately adjacent to each other. For example, “contiguous nucleobases” means nucleobases that are immediately adjacent to each other in a sequence.

“Designing” or “Designed to” refer to the process of designing a compound that specifically hybridizes with a selected nucleic acid molecule.

“Diluent” means an ingredient in a composition that lacks pharmacological activity, but is pharmaceutically necessary or desirable. For example, the diluent in an injected composition can be a liquid, e.g. saline solution.

“Differently modified” means chemical modifications or chemical substituents that are different from one another, including absence of modifications. Thus, for example, a MOE nucleoside and an unmodified DNA nucleoside are “differently modified,” even though the DNA nucleoside is unmodified. Likewise, DNA and RNA are “differently modified,” even though both are naturally-occurring unmodified nucleosides. Nucleosides that are the same but for comprising different nucleobases are not differently modified. For example, a nucleoside comprising a 2′-OMe modified sugar and an unmodified adenine nucleobase and a nucleoside comprising a 2′-OMe modified sugar and an unmodified thymine nucleobase are not differently modified.

“Dose” means a specified quantity of a compound or pharmaceutical agent provided in a single administration, or in a specified time period. In certain embodiments, a dose may be administered in two or more boluses, tablets, or injections. For example, in certain embodiments, where subcutaneous_administration is desired, the desired dose may require a volume not easily accommodated by a single injection. In such embodiments, two or more injections may be used to achieve the desired dose. In certain embodiments, a dose may be administered in two or more injections to minimize injection site reaction in an individual. In other embodiments, the compound or pharmaceutical agent is administered by infusion over an extended period of time or continuously. Doses may be stated as the amount of pharmaceutical agent per hour, day, week or month.

“Dosing regimen” is a combination of doses designed to achieve one or more desired effects.

“Double-stranded antisense compound” means an antisense compound comprising two oligomeric compounds that are complementary to each other and form a duplex, and wherein one of the two said oligomeric compounds comprises an oligonucleotide.

“Effective amount” means the amount of compound sufficient to effectuate a desired physiological outcome in an individual in need of the compound. The effective amount may vary among individuals depending on the health and physical condition of the individual to be treated, the taxonomic group of the individuals to be treated, the formulation of the composition, assessment of the individual's medical condition, and other relevant factors.

“Efficacy” means the ability to produce a desired effect.

“Expression” includes all the functions by which a gene's coded information is converted into structures present and operating in a cell. Such structures include, but are not limited to, the products of transcription and translation.

“Gapmer” means an oligonucleotide comprising an internal region having a plurality of nucleosides that support RNase H cleavage positioned between external regions having one or more nucleosides, wherein the nucleosides comprising the internal region are chemically distinct from the nucleoside or nucleosides comprising the external regions. The internal region may be referred to as the “gap” and the external regions may be referred to as the “wings.”

“Hybridization” means the annealing of oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases. In certain embodiments, complementary nucleic acid molecules include, but are not limited to, an antisense compound and a nucleic acid target. In certain embodiments, complementary nucleic acid molecules include, but are not limited to, an oligonucleotide and a nucleic acid target.

“Immediately adjacent” means there are no intervening elements between the immediately adjacent elements of the same kind (e.g. no intervening nucleobases between the immediately adjacent nucleobases).

“Individual” means a human or non-human animal selected for treatment or therapy.

“Inhibiting the expression or activity” refers to a reduction or blockade of the expression or activity relative to the expression of activity in an untreated or control sample and does not necessarily indicate a total elimination of expression or activity.

“Internucleoside linkage” means a group or bond that forms a covalent linkage between adjacent nucleosides in an oligonucleotide. “Modified internucleoside linkage” means any internucleoside linkage other than a naturally occurring, phosphate internucleoside linkage. Non-phosphate linkages are referred to herein as modified internucleoside linkages.

“Lengthened oligonucleotides” are those that have one or more additional nucleosides relative to an oligonucleotide disclosed herein, e.g. a parent oligonucleotide.

“Linked nucleosides” means adjacent nucleosides linked together by an internucleoside linkage.

“Linker-nucleoside” means a nucleoside that links an oligonucleotide to a conjugate moiety. Linker-nucleosides are located within the conjugate linker of a compound. Linker-nucleosides are not considered part of the oligonucleotide portion of a compound even if they are contiguous with the oligonucleotide.

“Mismatch” or “non-complementary” means a nucleobase of a first oligonucleotide that is not complementary to the corresponding nucleobase of a second oligonucleotide or target nucleic acid when the first and second oligonucleotides are aligned. For example, nucleobases including but not limited to a universal nucleobase, inosine, and hypoxanthine, are capable of hybridizing with at least one nucleobase but are still mismatched or non-complementary with respect to nucleobase to which it hybridized. As another example, a nucleobase of a first oligonucleotide that is not capable of hybridizing to the corresponding nucleobase of a second oligonucleotide or target nucleic acid when the first and second oligonucleotides are aligned is a mismatch or non-complementary nucleobase.

“Modulating” refers to changing or adjusting a feature in a cell, tissue, organ or organism. For example, modulating PNPLA3 RNA can mean to increase or decrease the level of PNPLA3 RNA and/or PNPLA3 protein in a cell, tissue, organ or organism. A “modulator” effects the change in the cell, tissue, organ or organism. For example, a PNPLA3 compound can be a modulator that decreases the amount of PNPLA3 RNA and/or PNPLA3 protein in a cell, tissue, organ or organism.

“MOE” means methoxyethyl.

“Monomer” refers to a single unit of an oligomer. Monomers include, but are not limited to, nucleosides and nucleotides.

“Motif” means the pattern of unmodified and/or modified sugar moieties, nucleobases, and/or internucleoside linkages, in an oligonucleotide.

“Natural” or “naturally occurring” means found in nature.

“Non-bicyclic modified sugar” or “non-bicyclic modified sugar moiety” means a modified sugar moiety that comprises a modification, such as a substituent, that does not form a bridge between two atoms of the sugar to form a second ring.

“Nucleic acid” refers to molecules composed of monomeric nucleotides. A nucleic acid includes, but is not limited to, ribonucleic acids (RNA), deoxyribonucleic acids (DNA), single-stranded nucleic acids, and double-stranded nucleic acids.

“Nucleobase” means a heterocyclic moiety capable of pairing with a base of another nucleic acid. As used herein a “naturally occurring nucleobase” is adenine (A), thymine (T), cytosine (C), uracil (U), and guanine (G). A “modified nucleobase” is a naturally occurring nucleobase that is chemically modified. A

“universal base” or “universal nucleobase” is a nucleobase other than a naturally occurring nucleobase and modified nucleobase, and is capable of pairing with any nucleobase.

“Nucleobase sequence” means the order of contiguous nucleobases in a nucleic acid or oligonucleotide independent of any sugar or internucleoside linkage.

“Nucleoside” means a compound comprising a nucleobase and a sugar moiety. The nucleobase and sugar moiety are each, independently, unmodified or modified. “Modified nucleoside” means a nucleoside comprising a modified nucleobase and/or a modified sugar moiety. Modified nucleosides include abasic nucleosides, which lack a nucleobase.

“Oligomeric compound” means a compound comprising a single oligonucleotide and optionally one or more additional features, such as a conjugate group or terminal group.

“Oligonucleotide” means a polymer of linked nucleosides each of which can be modified or unmodified, independent one from another. Unless otherwise indicated, oligonucleotides consist of 8-80 linked nucleosides. “Modified oligonucleotide” means an oligonucleotide, wherein at least one sugar, nucleobase, or internucleoside linkage is modified. “Unmodified oligonucleotide” means an oligonucleotide that does not comprise any sugar, nucleobase, or internucleoside modification.

“Parent oligonucleotide” means an oligonucleotide whose sequence is used as the basis of design for more oligonucleotides of similar sequence but with different lengths, motifs, and/or chemistries. The newly designed oligonucleotides may have the same or overlapping sequence as the parent oligonucleotide.

“Parenteral administration” means administration through injection or infusion. Parenteral administration includes subcutaneous administration, intravenous administration, intramuscular administration, intraarterial administration, intraperitoneal administration, or intracranial administration, e.g. intrathecal or intracerebroventricular administration.

“Patatin like phospholipase domain containing 3,” abbreviated as PNPLA3 and also referred to as adiponutrin (ADPN), acylglycerol O-acyltransferase, calcium-independent phospholipase A2-epsilon (iPLA2-epsilon), hypothetical protein dJ796I17.1, or DJ796I17.1, is a 481-amino acid protein encoded by the Pnpla3 gene. PNPLA3 has hydrolase activity towards triglycerides and retinyl esters, promoting lipid droplet remodeling in hepatocytes and hepatic stellate cells. As described herein, PNPLA3 is a member of the patatin-like phospholipase domain-containing family that is expressed on the ER and on lipid droplets. In humans, PNPLA3 is highly expressed in the liver. As used herein, “PNPLA3” can refer to any nucleic acid or protein of PNPLA3. “PNPLA3 nucleic acid” means any nucleic acid encoding PNPLA3. For example, in certain embodiments, a PNPLA3 nucleic acid includes a DNA sequence encoding PNPLA3, an RNA sequence transcribed from DNA encoding PNPLA3 (including genomic DNA comprising introns and exons), and an mRNA sequence encoding PNPLA3. “PNPLA3 mRNA” means an mRNA encoding a PNPLA3 protein. The target may be referred to in either upper or lower case.

“PNPLA3 specific inhibitor” refers to any agent capable of specifically inhibiting PNPLA3 RNA and/or PNPLA3 protein expression or activity at the molecular level. For example, PNPLA3 specific inhibitors include nucleic acids (including antisense compounds), peptides, antibodies, and other agents capable of inhibiting the expression of PNPLA3 RNA and/or PNPLA3 protein.

“Pharmaceutically acceptable carrier or diluent” means any substance suitable for use in administering to an individual. For example, a pharmaceutically acceptable carrier can be a sterile aqueous solution, such as PBS or water-for-injection.

“Pharmaceutically acceptable salts” means physiologically and pharmaceutically acceptable salts of compounds, such as oligomeric compounds or oligonucleotides, i.e., salts that retain the desired biological activity of the parent compound and do not impart undesired toxicological effects thereto.

“Pharmaceutical agent” means a compound that provides a therapeutic benefit when administered to an individual.

“Pharmaceutical composition” means a mixture of substances suitable for administering to an individual. For example, a pharmaceutical composition may comprise one or more compounds or salt thereof and a sterile aqueous solution.

“Phosphorothioate linkage” means a modified phosphate linkage in which one of the non-bridging oxygen atoms is replaced with a sulfur atom. A phosphorothioate internucleoside linkage is a modified internucleoside linkage.

“Phosphorus moiety” means a group of atoms comprising a phosphorus atom. In certain embodiments, a phosphorus moiety comprises a mono-, di-, or tri-phosphate, or phosphorothioate.

“Portion” means a defined number of contiguous (i.e., linked) nucleobases of a nucleic acid. In certain embodiments, a portion is a defined number of contiguous nucleobases of a target nucleic acid. In certain embodiments, a portion is a defined number of contiguous nucleobases of an oligomeric compound.

“Prevent” refers to delaying or forestalling the onset, development or progression of a disease, disorder, or condition for a period of time from minutes to indefinitely.

“Prodrug” means a compound in a form outside the body which, when administered to an individual, is metabolized to another form within the body or cells thereof. In certain embodiments, the metabolized form is the active, or more active, form of the compound (e.g., drug). Typically conversion of a prodrug within the body is facilitated by the action of an enzyme(s) (e.g., endogenous or viral enzyme) or chemical(s) present in cells or tissues, and/or by physiologic conditions.

“Reduce” means to bring down to a smaller extent, size, amount, or number.

“RefSeq No.” is a unique combination of letters and numbers assigned to a sequence to indicate the sequence is for a particular target transcript (e.g., target gene). Such sequence and information about the target gene (collectively, the gene record) can be found in a genetic sequence database. Genetic sequence_databases include the NCBI Reference Sequence database, GenBank, the European Nucleotide Archive, and the DNA Data Bank of Japan (the latter three forming the International Nucleotide Sequence Database Collaboration or INSDC).

“Region” is defined as a portion of the target nucleic acid having at least one identifiable structure, function, or characteristic.

“RNAi compound” means an antisense compound that acts, at least in part, through RISC or Ago2, but not through RNase H, to modulate a target nucleic acid and/or protein encoded by a target nucleic acid. RNAi compounds include, but are not limited to double-stranded siRNA, single-stranded RNA (ssRNA), and microRNA, including microRNA mimics.

“Segments” are defined as smaller or sub-portions of regions within a nucleic acid.

“Side effects” means physiological disease and/or conditions attributable to a treatment other than the desired effects. In certain embodiments, side effects include injection site reactions, liver function test abnormalities, renal function abnormalities, liver toxicity, renal toxicity, central nervous system abnormalities, myopathies, and malaise. For example, increased aminotransferase levels in serum may indicate liver toxicity or liver function abnormality. For example, increased bilirubin may indicate liver toxicity or liver function abnormality.

“Single-stranded” in reference to a compound means the compound has only one oligonucleotide. “Self-complementary” means an oligonucleotide that at least partially hybridizes to itself. A compound consisting of one oligonucleotide, wherein the oligonucleotide of the compound is self-complementary, is a single-stranded compound. A single-stranded compound may be capable of binding to a complementary compound to form a duplex.

“Sites” are defined as unique nucleobase positions within a target nucleic acid.

“Specifically hybridizable” refers to an oligonucleotide having a sufficient degree of complementarity between the oligonucleotide and a target nucleic acid to induce a desired effect, while exhibiting minimal or no effects on non-target nucleic acids. In certain embodiments, specific hybridization occurs under physiological conditions.

“Specifically inhibit” with reference to a target nucleic acid means to reduce or block expression of the target nucleic acid while exhibiting fewer, minimal, or no effects on non-target nucleic acids. Reduction does not necessarily indicate a total elimination of the target nucleic acid's expression.

“Standard cell assay” means assay(s) described in the Examples and reasonable variations thereof.

“Standard in vivo experiment” means the procedure(s) described in the Example(s) and reasonable variations thereof.

“Stereorandom chiral center” in the context of a population of molecules of identical molecular formula means a chiral center having a random stereochemical configuration. For example, in a population of molecules comprising a stereorandom chiral center, the number of molecules having the (S) configuration of the stereorandom chiral center may be but is not necessarily the same as the number of molecules having the (R) configuration of the stereorandom chiral center. The stereochemical configuration of a chiral center is considered random when it is the result of a synthetic method that is not designed to control the stereochemical configuration. In certain embodiments, a stereorandom chiral center is a stereorandom phosphorothioate internucleoside linkage.

“Sugar moiety” means an unmodified sugar moiety or a modified sugar moiety. “Unmodified sugar moiety” or “unmodified sugar” means a 2′-OH(H) ribosyl moiety, as found in RNA (an “unmodified RNA sugar moiety”), or a 2′-H(H) moiety, as found in DNA (an “unmodified DNA sugar moiety”). “Modified sugar moiety” or “modified sugar” means a modified furanosyl sugar moiety or a sugar surrogate. “Modified furanosyl sugar moiety” means a furanosyl sugar comprising a non-hydrogen substituent in place of at least one hydrogen or hydroxyl of an unmodified sugar moiety. In certain embodiments, a modified furanosyl sugar moiety is a 2′-substituted sugar moiety. Such modified furanosyl sugar moieties include bicyclic sugars and non-bicyclic sugars.

“Sugar surrogate” means a modified sugar moiety having other than a furanosyl moiety that can link a nucleobase to another group, such as an internucleoside linkage, conjugate group, or terminal group in an oligonucleotide. Modified nucleosides comprising sugar surrogates can be incorporated into one or more positions within an oligonucleotide and such oligonucleotides are capable of hybridizing to complementary compounds or nucleic acids.

“Synergy” or “synergize” refers to an effect of a combination that is greater than additive of the effects of each component alone at the same doses.

“Target gene” refers to a gene encoding a target.

“Targeting” means the specific hybridization of a compound to a target nucleic acid in order to induce a desired effect.

“Target nucleic acid,” “target RNA,” “target RNA transcript” and “nucleic acid target” all mean a nucleic acid capable of being targeted by compounds described herein.

“Target region” means a portion of a target nucleic acid to which one or more compounds is targeted.

“Target segment” means the sequence of nucleotides of a target nucleic acid to which a compound is targeted. “5′ target site” refers to the 5′-most nucleotide of a target segment. “3′ target site” refers to the 3′-most nucleotide of a target segment.

“Terminal group” means a chemical group or group of atoms that is covalently linked to a terminus of an oligonucleotide.

“Therapeutically effective amount” means an amount of a compound, pharmaceutical agent, or composition that provides a therapeutic benefit to an individual.

“Treat” refers to administering a compound or pharmaceutical composition to an animal in order to effect an alteration or improvement of a disease, disorder, or condition in the animal.

### Certain Embodiments

Certain embodiments provide methods, compounds and compositions for inhibiting PNPLA3 (PNPLA3) expression.

Certain embodiments provide compounds targeted to a PNPLA3 nucleic acid. In certain embodiments, the PNPLA3 nucleic acid has the sequence set forth in RefSeq or GENBANK Accession No. NM_025225.2 (incorporated by reference, disclosed herein as SEQ ID NO: 1); NC_000022.11 truncated from nucleotides 43921001 to U.S. Pat. No. 43,954,500 (incorporated by reference, disclosed herein as SEQ ID NO: 2); AK123806.1(incorporated by reference, disclosed herein as SEQ ID NO: 3); BQ686328.1 (incorporated by reference, disclosed herein as SEQ ID NO: 4); BF762711.1 (incorporated by reference, disclosed herein as SEQ ID NO: 5); DA290491.1 (incorporated by reference, disclosed herein as SEQ ID NO: 6); and the sequences listed as SEQ ID Nos 7, 8, 9, and 10. In certain embodiments, the compound is an antisense compound or oligomeric compound. In certain embodiments, the compound is single-stranded. In certain embodiments, the compound is double-stranded.

In certain embodiments, the compound comprises a modified oligonucleotide 16 linked nucleosides in length. In certain embodiments, the compound is an antisense compound or oligomeric compound.

Certain embodiments provide a compound comprising a modified oligonucleotide 12 to 30 linked nucleosides in length and having a nucleobase sequence comprising the nucleobase sequence of any one of SEQ ID NOs: 17-2169. In certain embodiments, the compound is an antisense compound or oligomeric compound. In certain embodiments, the compound is single-stranded. In certain embodiments, the compound is double-stranded. In certain embodiments, the modified oligonucleotide is 16 to 30 linked nucleosides in length.

Certain embodiments provide a compound comprising a modified oligonucleotide consisting of the nucleobase sequence of any one of SEQ ID NOs: 17-2169. In certain embodiments, the compound is an antisense compound or oligomeric compound. In certain embodiments, the compound is single-stranded. In certain embodiments, the compound is double-stranded.

Certain embodiments provide a compound comprising a modified oligonucleotide 12 to 30 linked nucleosides in length and complementary within nucleobases 5567-5642, 5644-5731, 5567-5731, 5567-5620, 13697-13733, 20553-20676, 20664-20824, 20553-20824, and 25844-25912 of SEQ ID NO: 2, wherein said modified oligonucleotide is at least 85%, at least 90%, at least 95%, or 100% complementary to SEQ ID NO: 2. In certain embodiments, the compound is an antisense compound or oligomeric compound. In certain embodiments, the compound is single-stranded. In certain embodiments, the compound is double-stranded. In certain embodiments, the modified oligonucleotide is 16 to 30 linked nucleosides in length.

In certain embodiments, compounds target nucleotides 5567-5620 ofa PNPLA3 nucleic acid. In certain embodiments, compounds target within nucleotides 5567-5642, 5644-5731, 5567-5731, 5567-5620 of a PNPLA3 nucleic acid having the nucleobase sequence of SEQ ID NO: 2. In certain embodiments, compounds have at least an 8, 9, 10, 11, 12, 13, 14, 15, or 16 contiguous nucleobase portion complementary to an equal length portion within nucleotides 5567-5642, 5644-5731, 5567-5731, 5567-5620 of a PNPLA3 nucleic acid having the nucleobase sequence of SEQ ID NO: 2. In certain embodiments, these compounds are antisense compounds, oligomeric compounds, or oligonucleotides.

In certain embodiments, a compound comprises a modified oligonucleotide 12 to 30 linked nucleosides in length and having a nucleobase sequence comprising at least an 8, 9, 10, 11, 12, 13, 14, 15, or 16 contiguous nucleobase portion any one of SEQ ID NOs: 1089, 1757, 141, 1982, 330, 1665, 408, 830, and 899. In certain embodiments, the modified oligonucleotide is 16 to 30 linked nucleosides in length.

In certain embodiments, a compound comprises a modified oligonucleotide 12 to 30 linked nucleosides in length and having a nucleobase sequence comprising any one of SEQ ID NOs: 1089, 1757, 141, 1982, 330, 1665, 408, 830, and 899. In certain embodiments, the modified oligonucleotide is 16 to 30 linked nucleosides in length.

In certain embodiments, a compound comprises a modified oligonucleotide having a nucleobase sequence consisting of any one of SEQ ID NOs: 1089, 1757, 141, 1982, 330, 1665, 408, 830, and 899.

In certain embodiments, compounds targeted to PNPLA3 is ION 975616, 994284, 975605, 994282, 975613, 975617, 975735, 975736, or 975612. Out of over 2,384 compounds that were screened as described in the Examples section below, ION 975616, 994284, 975605, 994282, 975613, 975617, 975735, 975736, and 975612 emerged as the top lead compounds.

In certain embodiments, any of the foregoing modified oligonucleotides comprises at least one modified internucleoside linkage, at least one modified sugar, and/or at least one modified nucleobase.

In certain embodiments, any of the foregoing modified oligonucleotides comprises at least one modified sugar. In certain embodiments, at least one modified sugar comprises a 2′-O-methoxyethyl group. In certain embodiments, at least one modified sugar is a bicyclic sugar, such as a 4′—CH(CH3)—O—2′ group, a 4′-CH2—O—2′ group, or a 4′—(CH2)2—O—2′ group.

In certain embodiments, the modified oligonucleotide comprises at least one modified internucleoside linkage, such as a phosphorothioate internucleoside linkage.

In certain embodiments, any of the foregoing modified oligonucleotides comprises at least one modified nucleobase, such as 5-methylcytosine.

In certain embodiments, any of the foregoing modified oligonucleotides comprises:
a gap segment consisting of linked deoxynucleosides;
a 5′ wing segment consisting of linked nucleosides; and
a 3′ wing segment consisting of linked nucleosides;
   wherein the gap segment is positioned between the 5′ wing segment and the 3′ wing segment and wherein each nucleoside of each wing segment comprises a modified sugar. In certain embodiments, the modified oligonucleotide is 12 to 30 linked nucleosides in length having a nucleobase sequence comprising the sequence recited in any one of SEQ ID NOs: 1089, 1757, 141, 1982, 330, 1665, 408, 830, and 899. In certain embodiments, the modified oligonucleotide is 16 to 30 linked nucleosides in length having a nucleobase sequence comprising the sequence recited in any one of SEQ ID NOs: 1089, 1757, 141, 1982, 330, 1665, 408, 830, and 899. In certain embodiments, the modified oligonucleotide is 16 linked nucleosides in length having a nucleobase sequence consisting of the sequence recited in any one of SEQ ID NOs: 1089, 1757, 141, 1982, 330, 1665, 408, 830, and 899.

In certain embodiments, a compound comprises or consists of a modified oligonucleotide 12-30 linked nucleobases in length having a nucleobase sequence comprising the sequence recited in any one of SEQ ID NOs: 1089, 1757, 141, 1982, 330, 1665, 408, 830, and 899, wherein the modified oligonucleotide comprises
a gap segment consisting of ten linked deoxynucleosides;
a 5′ wing segment consisting of three linked nucleosides; and
a 3′ wing segment consisting of three linked nucleosides;
   wherein the gap segment is positioned between the 5′ wing segment and the 3′ wing segment, wherein each nucleoside of each wing segment comprises a cEt sugar; wherein each internucleoside linkage is a phosphorothioate linkage and wherein each cytosine is a 5-methylcytosine. In certain embodiments, the modified oligonucleotide consists of 16-30 linked nucleosides. In certain embodiments, the modified oligonucleotide consists of 16 linked nucleosides.

In certain embodiments, a compound comprises or consists of a modified oligonucleotide, wherein the modified oligonucleotide is 16 linked nucleosides in length and consists of the sequence of SEQ ID NO: 1089, wherein the modified oligonucleotide comprises:
a gap segment consisting of ten linked deoxynucleosides;
a 5′ wing segment consisting of three linked nucleosides; and
a 3′ wing segment consisting of three linked nucleosides;

wherein the gap segment is positioned between the 5′ wing segment and the 3′ wing segment, wherein each nucleoside of each wing segment comprises a cEt sugar; wherein each internucleoside linkage is a phosphorothioate linkage; and wherein each cytosine is a 5-methylcytosine.

In certain embodiments, a compound consists of a modified oligonucleotide and a conjugate group, wherein the modified oligonucleotide is 16 linked nucleosides in length and consists of the sequence of SEQ ID NO: 1089, wherein the modified oligonucleotide comprises:
a gap segment consisting of ten linked deoxynucleosides;
a 5′ wing segment consisting of three linked nucleosides; and
a 3′ wing segment consisting of three linked nucleosides;

wherein the gap segment is positioned between the 5′ wing segment and the 3′ wing segment, wherein each nucleoside of each wing segment comprises a cEt sugar; wherein each internucleoside linkage is a phosphorothioate linkage; wherein each cytosine is a 5-methylcytosine; and wherein the conjugate group is positioned at the 5′end of the modified oligonucleotide and is

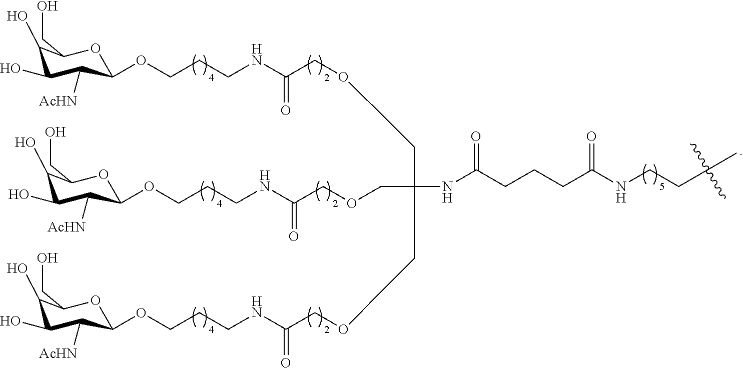

In some embodiments, the compound targeted to PNPLA3, in an individual having an I148M mutation, comprises a modified oligonucleotide, wherein the modified oligonucleotide has a nucleobase sequence at least 90% identical to SEQ ID NO: 115. In some embodiments, the compound targeted to PNPLA3, in an individual having an I148M mutation, comprises a modified oligonucleotide, wherein the modified 20 oligonucleotide has a nucleobase sequence at least 90% identical to any one of SEQ ID NOs: 2170-2172.

In some embodiments, the compound targeted to PNPLA3, in an individual having an I148M mutation, consists of the sequence of SEQ ID NO: 115, wherein the modified oligonucleotide comprises:
a gap segment consisting of ten linked deoxynucleosides;
a 5′ wing segment consisting of three linked nucleosides; and
a 3′ wing segment consisting of three linked nucleosides;
   wherein the gap segment is positioned between the 5′ wing segment and the 3′ wing segment, wherein each nucleoside of each wing segment comprises a cEt sugar; wherein each internucleoside linkage is a phosphorothioate linkage; and wherein each cytosine is a 5-methylcytosine. In some embodiments, the compound targeted to PNPLA3, in an individual having an 1148M mutation, further comprises a conjugate group, wherein the conjugate group is positioned at the 5′end of the modified oligonucleotide and is

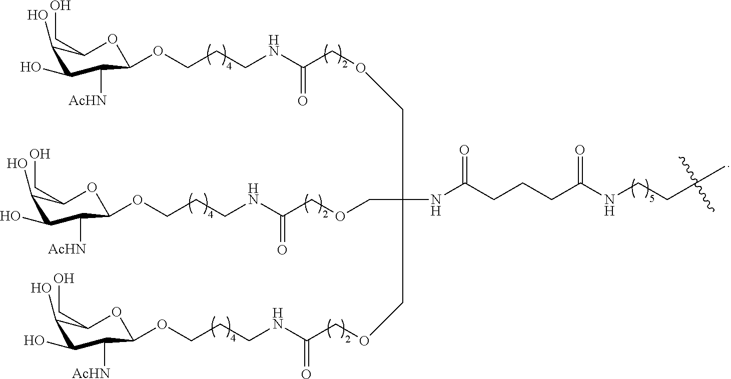

In certain embodiments, a compound comprises or consists of ION 916333 or salt thereof, having the following chemical structure:

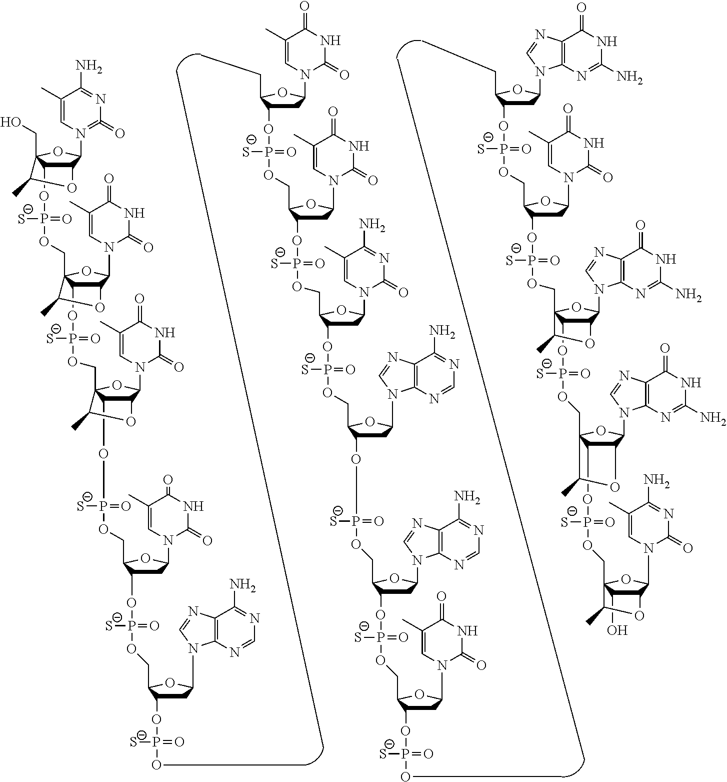

In certain embodiments, a compound comprises or consists of ION 975616 or salt thereof, having the

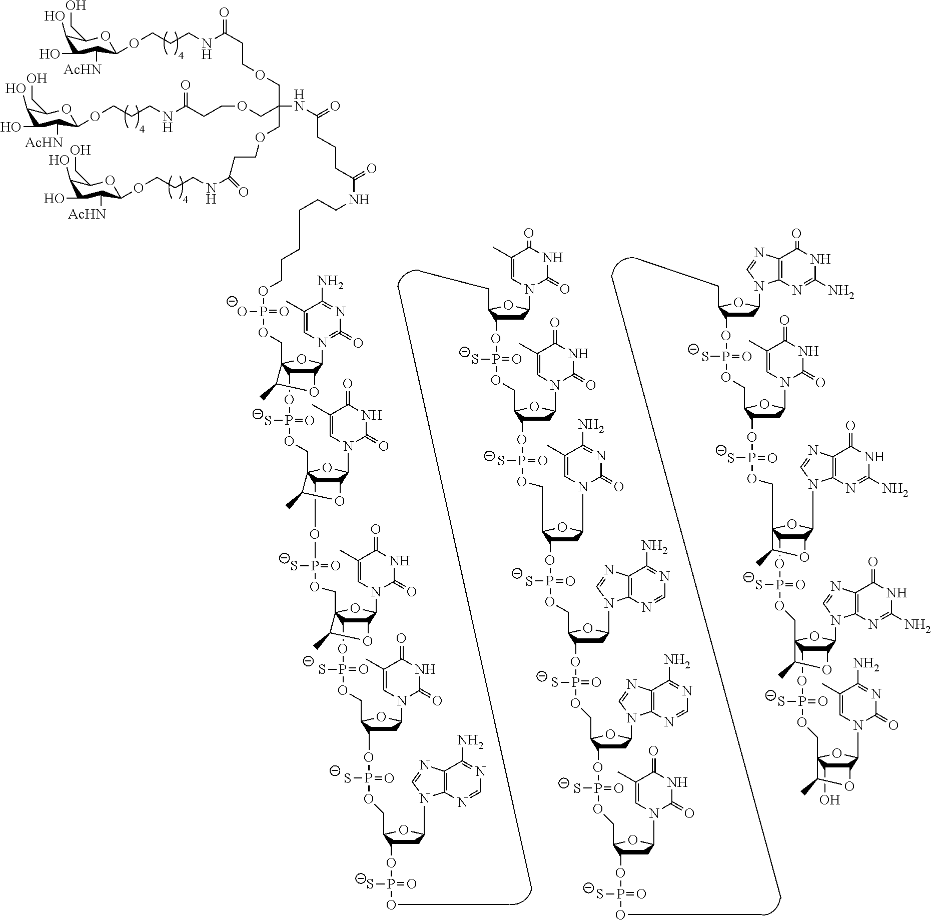

In certain embodiments, a compound comprises or consists of the sodium salt of 975616, having the

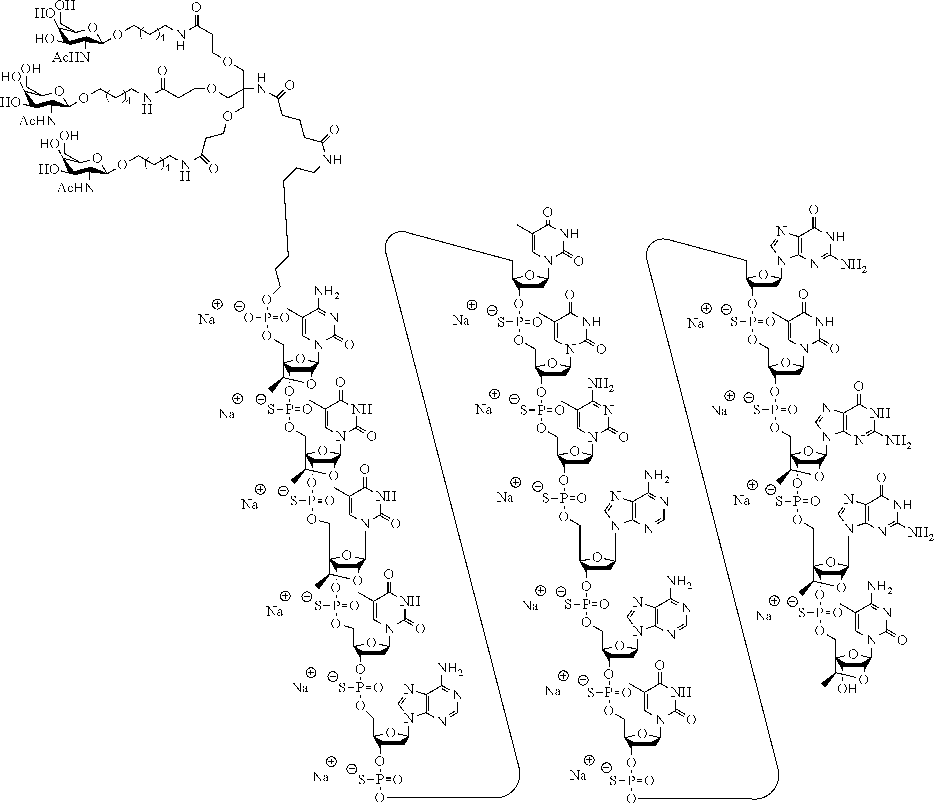

In certain embodiments, a compound comprises or consists of ION 975613 or salt thereof, having the

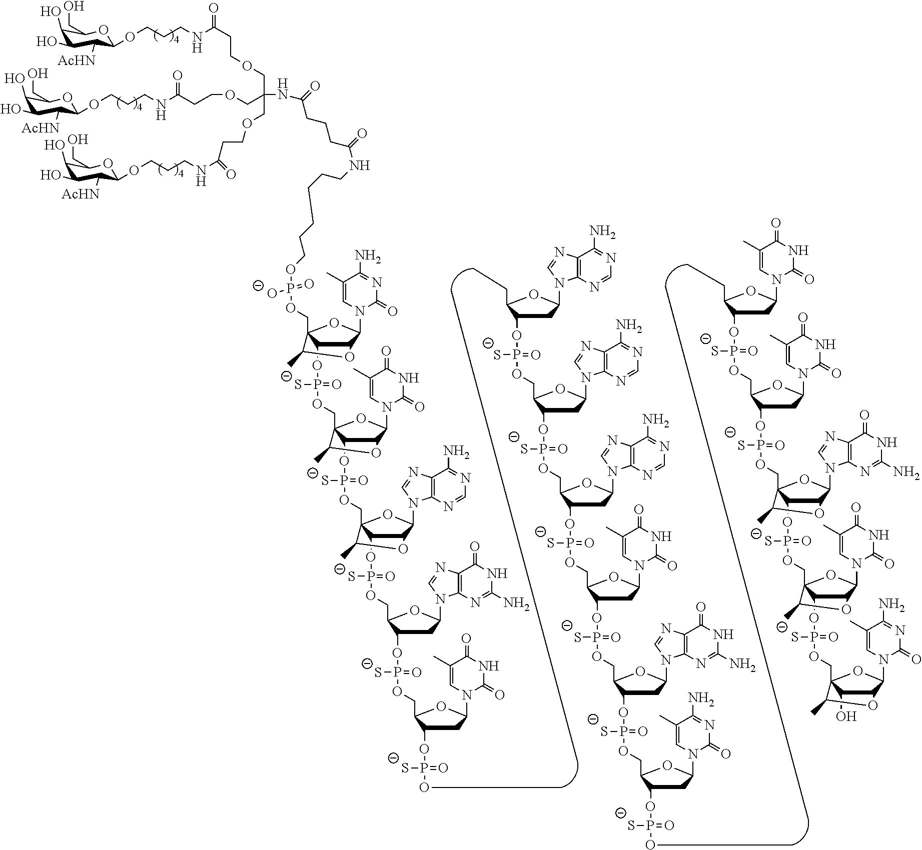

In certain embodiments, a compound comprises or consists of the sodium salt of ION 975613, having

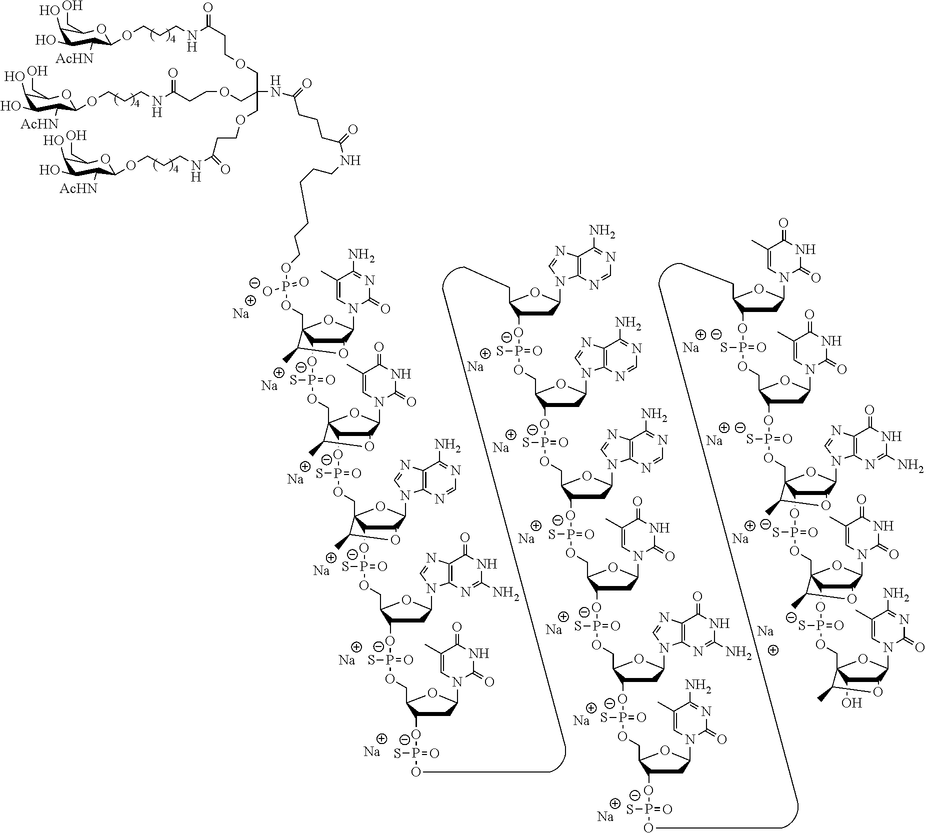

In certain embodiments, a compound comprises or consists of ION 975612 or salt thereof, having the

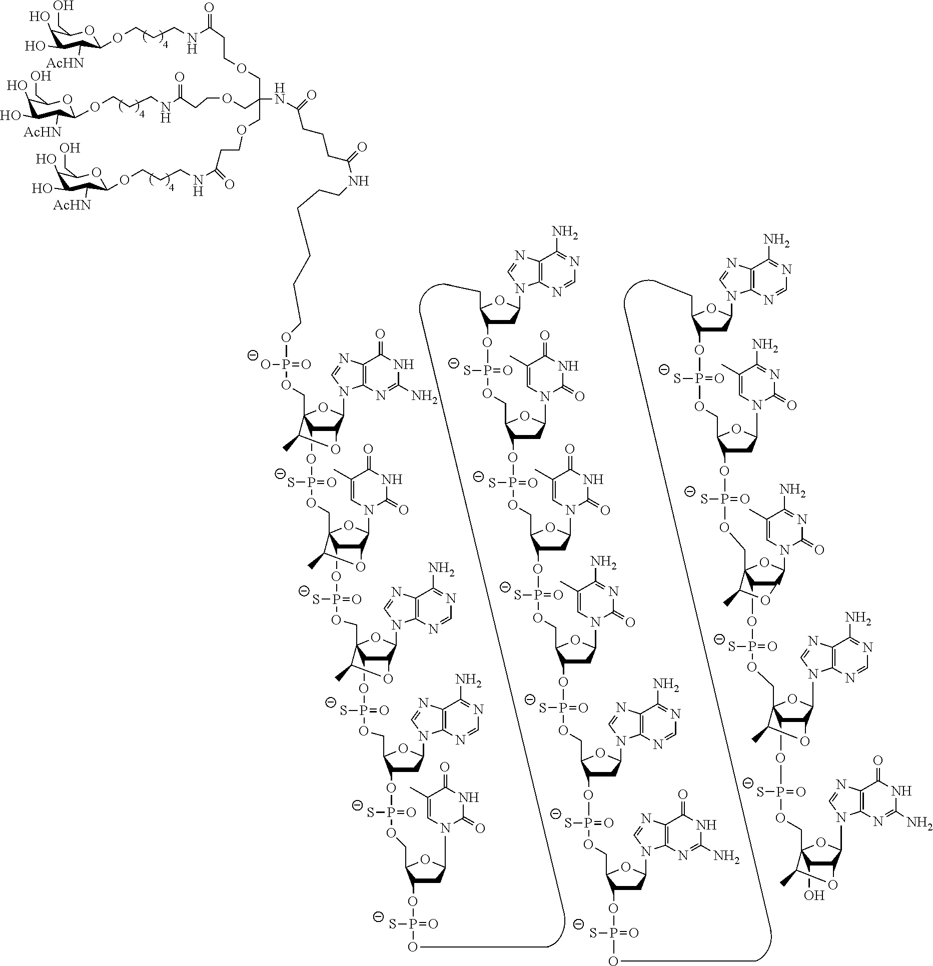

In certain embodiments, a compound comprises or consists of the sodium salt of ION 975612, having

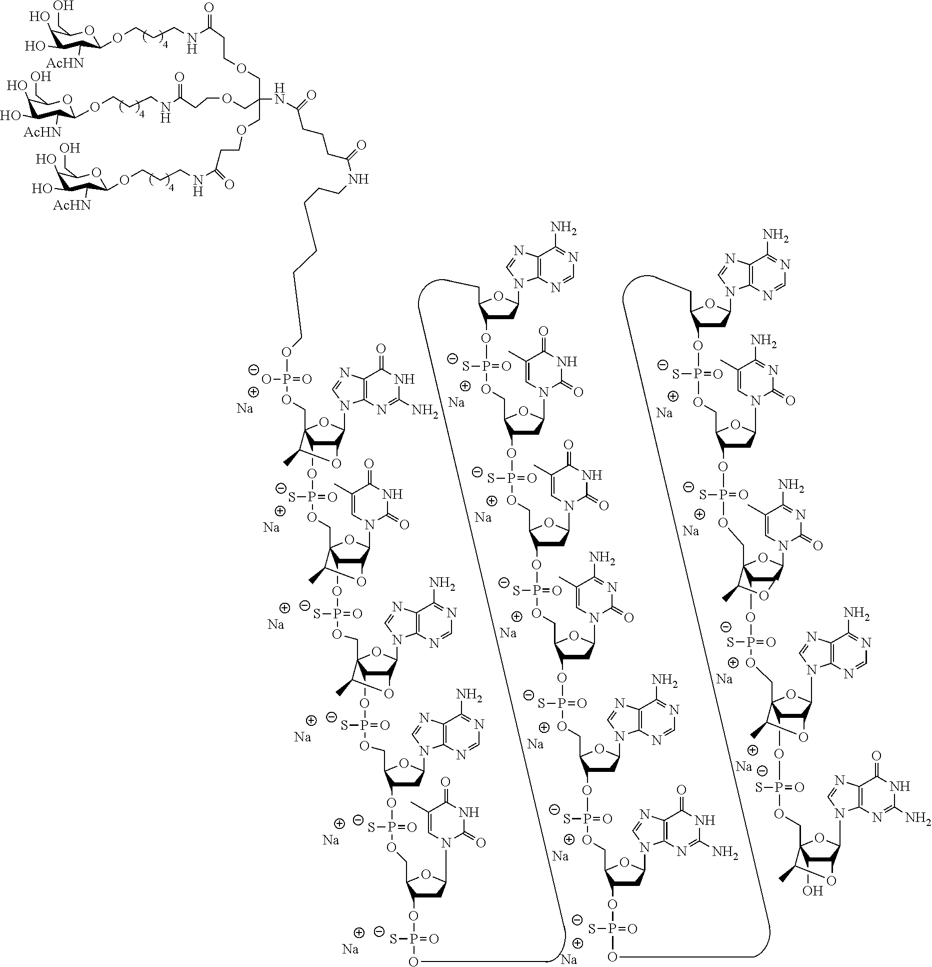

In certain embodiments, a compound comprises or consists of ION 916789 or salt thereof, having the

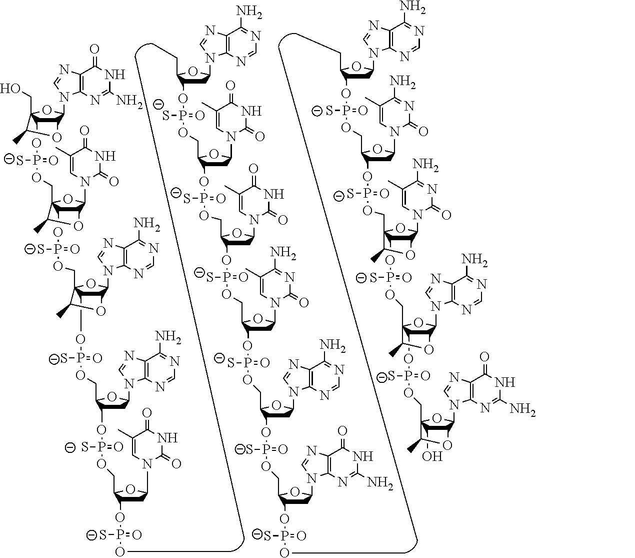

In certain embodiments, a compound comprises or consists of the sodium salt of ION 916789, having

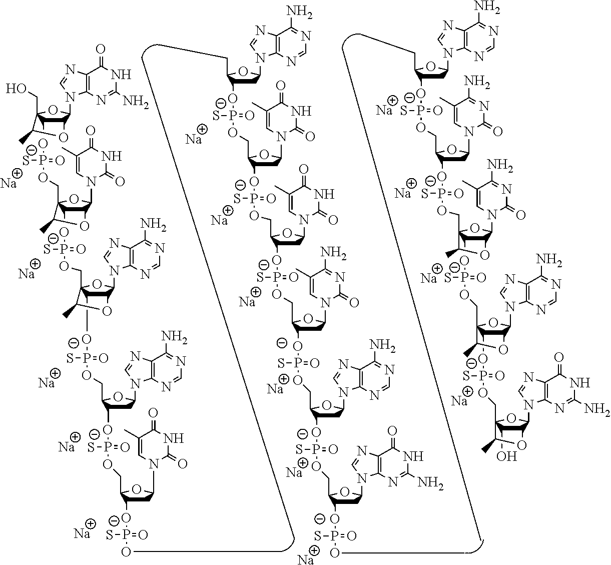

In certain embodiments, a compound comprises or consists of ION 916602 or salt thereof, having the

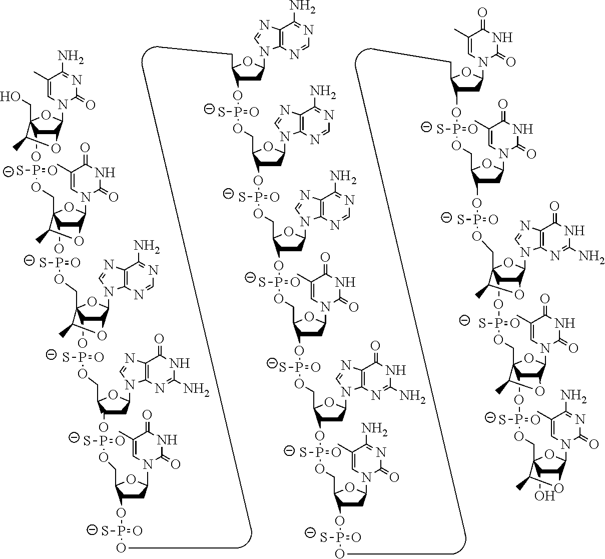

In certain embodiments, a compound comprises or consists of the sodium salt of ION 916602, having the following chemical structure:

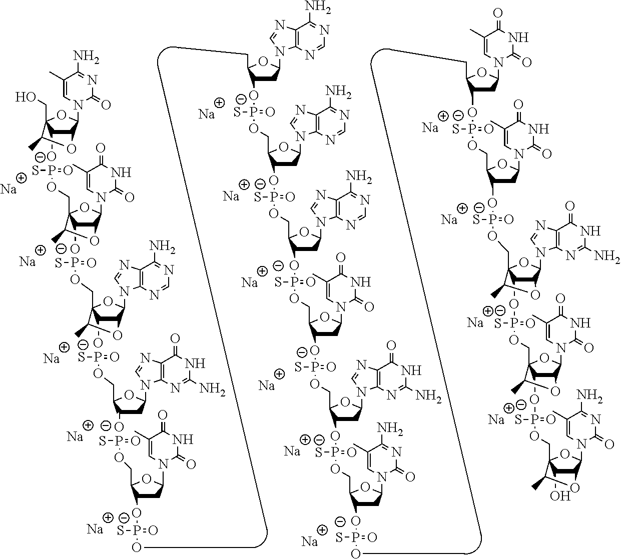

In any of the foregoing embodiments, the compound or oligonucleotide can be at least 85%, at least 90%, at least 95%, at least 98%, at least 99%, or 100% complementary to a nucleic acid encoding PNPLA3.

In any of the foregoing embodiments, the compound can be single-stranded. In certain embodiments, the compound comprises deoxyribonucleotides. In certain embodiments, the compound is double-stranded. In certain embodiments, the compound is double-stranded and comprises ribonucleotides. In any of the foregoing embodiments, the compound can be an antisense compound or oligomeric compound.

In any of the foregoing embodiments, the compound can be 8 to 80, 10 to 30, 12 to 50, 13 to 30, 13 to 50, 14 to 30, 14 to 50, 15 to 30, 15 to 50, 16 to 30, 16 to 50, 17 to 30, 17 to 50, 18 to 22, 18 to 24, 18 to 30, 18 to 50, 19 to 22, 19 to 30, 19 to 50, or 20 to 30 linked nucleosides in length. In certain embodiments, the compound comprises or consists of an oligonucleotide.

In certain embodiments, a compound comprises a modified oligonucleotide described herein and a conjugate group. In certain embodiments, the conjugate group is linked to the modified oligonucleotide at the 5′ end of the modified oligonucleotide. In certain embodiments, the conjugate group is linked to the modified oligonucleotide at the 3′ end of the modified oligonucleotide. In certain embodiments, the conjugate group comprises at least one N-Acetylgalactosamine (GalNAc), at least two N-Acetylgalactosamines (GalNAcs), or at least three N-Acetylgalactosamines (GalNAcs).

In certain embodiments, compounds or compositions provided herein comprise a pharmaceutically acceptable salt of the modified oligonucleotide. In certain embodiments, the salt is a sodium salt. In certain embodiments, the salt is a potassium salt.

In certain embodiments, the compounds or compositions as described herein are active by virtue of having at least one of an in vitro IC₅₀ of less than 2 μM, less than 1.5 μM, less than 1 μM, less than 0.9 μM, less than 0.8 μM, less than 0.7 μM, less than 0.6 μM, less than 0.5 μM, less than 0.4 μM, less than 0.3 μM, less than 0.2 μM, less than 0.1 μM, less than 0.05 μM, less than 0.04 μM, less than 0.03 μM, less than 0.02 μM, or less than 0.01 μM.

In certain embodiments, the compounds or compositions as described herein are highly tolerable as demonstrated by having at least one of an increase in alanine transaminase (ALT) or aspartate transaminase (AST) value of no more than 4 fold, 3 fold, or 2 fold over control animals, or an increase in liver, spleen, or kidney weight of no more than 30%, 20%, 15%, 12%, 10%, 5%, or 2% compared to control animals. In certain embodiments, the compounds or compositions as described herein are highly tolerable as demonstrated by having no increase of ALT or AST over control animals. In certain embodiments, the compounds or compositions as described herein are highly tolerable as demonstrated by having no increase in liver, spleen, or kidney weight over control animals.

Certain embodiments provide a composition comprising the compound of any of the aforementioned embodiments or any pharmaceutically acceptable salt thereof and at least one of a pharmaceutically acceptable carrier or diluent. In certain embodiments, the composition has a viscosity less than about 40 centipoise (cP), less than about 30 centipose (cP), less than about 20 centipose (cP), less than about 15 centipose (cP), or less than about 10 centipose (cP). In certain embodiments, the composition having any of the aforementioned viscosities comprises a compound provided herein at a concentration of about 100 mg/mL, about 125 mg/mL, about 150 mg/mL, about 175 mg/mL, about 200 mg/mL, about 225 mg/mL, about 250 mg/mL, about 275 mg/mL, or about 300 mg/mL. In certain embodiments, the composition having any of the aforementioned viscosities and/or compound concentrations has a temperature of room temperature, or about 20° C., about 21° C., about 22° C., about 23° C., about 24° C., about 25° C., about 26° C., about 27° C., about 28° C., about 29° C., or about 30° C.

### Certain Indications

Certain embodiments provided herein relate to methods of inhibiting PNPLA3 expression, which can be useful for treating, preventing, or ameliorating a disease associated with PNPLA3 in an individual, by administration of a compound that targets PNPLA3. In certain embodiments, the compound can be a PNPLA3 specific inhibitor. In certain embodiments, the compound can be an antisense compound, an oligomeric compound, or an oligonucleotide targeted to PNPLA3.

Examples of diseases associated with PNPLA3 treatable, preventable, and/or ameliorable with the methods provided herein include liver disease, NAFLD, hepatic steatosis, non-alcoholic steatohepatitis (NASH), liver cirrhosis, hepatocellular carcinoma, alcoholic liver disease, alcoholic steatohepatitis (ASH), HCV hepatitis, chronic hepatitis, hereditary hemochromatosis, or primary sclerosing cholangitis. Certain compounds provided herein are directed to compounds and compositions that reduce liver damage, steatosis, liver fibrosis, liver inflammation, liver scarring or cirrhosis, liver failure, liver enlargement, elevated transaminases, or hepatic fat accumulation in an animal.

In certain embodiments, a method of treating, preventing, or ameliorating a disease associated with PNPLA3 in an individual comprises administering to the individual a compound comprising a PNPLA3 specific inhibitor, thereby treating, preventing, or ameliorating the disease. In certain embodiments, the individual is identified as having, or at risk of having, a disease associated with PNPLA3. In certain embodiments, the disease is a liver disease. In certain embodiments, the compound comprises an antisense compound targeted to PNPLA3. In certain embodiments, the compound comprises an oligonucleotide targeted to PNPLA3. In certain embodiments, a compound comprises a modified oligonucleotide 12 to 30 linked nucleosides in length and having a nucleobase sequence comprising at least 8 contiguous nucleobases of any of the nucleobase sequences of SEQ ID NOs: 17-2169. In certain embodiments, a compound comprises a modified oligonucleotide 12 to 30 linked nucleosides in length and having a nucleobase sequence comprising the nucleobase sequence of any one of SEQ ID NOs: 17-2169. In certain embodiments, a compound comprises a modified oligonucleotide consisting of the nucleobase sequence of any one of SEQ ID NOs: 17-2169. In certain embodiments, a compound comprises a modified oligonucleotide 16 to 30 linked nucleosides in length having a nucleobase sequence comprising any one of SEQ ID NOs: 1089, 1757, 141, 1982, 330, 1665, 408, 830, and 899. In certain embodiments, a compound comprises a modified oligonucleotide having a nucleobase sequence consisting of any one of SEQ ID NOs: 1089, 1757, 141, 1982, 330, 1665, 408, 830, and 899. In certain embodiments, the compound is ION 975616, 994284, 975605, 994282, 975613, 975617, 975735, 975736, or 975612. In any of the foregoing embodiments, the compound can be single-stranded or double-stranded. In any of the foregoing embodiments, the compound can be an antisense compound or oligomeric compound. In certain embodiments, the compound is administered to the individual parenterally. In certain embodiments, administering the compound improves, preserves, or prevents liver damage, steatosis, liver fibrosis, cirrhosis, elevated transaminases, or hepatic fat accumulation in an animal.

In certain embodiments, a method of treating, preventing, or ameliorating liver damage, steatosis, liver fibrosis, liver inflammation, liver scarring or cirrhosis, liver failure, liver enlargement, elevated transaminases, or hepatic fat accumulation in an animal comprises administering to the individual a compound comprising a PNPLA3 specific inhibitor, thereby treating, preventing, or ameliorating liver damage, steatosis, liver fibrosis, liver inflammation, liver scarring or cirrhosis, liver failure, liver enlargement, elevated transaminases, or hepatic fat accumulation. In certain embodiments, the compound comprises an antisense compound targeted to PNPLA3. In certain embodiments, the compound comprises an oligonucleotide targeted to PNPLA3. In certain embodiments, a compound comprises a modified oligonucleotide 12 to 30 linked nucleosides in length and having a nucleobase sequence comprising at least 8 contiguous nucleobases of any of the nucleobase sequences of SEQ ID NOs: 17-2169. In certain embodiments, a compound comprises a modified oligonucleotide 12 to 30 linked nucleosides in length and having a nucleobase sequence comprising the nucleobase sequence of any one of SEQ ID NOs: 17-2169. In certain embodiments, a compound comprises a modified oligonucleotide consisting of the nucleobase sequence of any one of SEQ ID NOs: 17-2169. In certain embodiments, a compound comprises a modified oligonucleotide 16 to 30 linked nucleosides in length having a nucleobase sequence comprising any one of SEQ ID NOs: 1089, 1757, 141, 1982, 330, 1665, 408, 830, and 899. In certain embodiments, a compound comprises a modified oligonucleotide having a nucleobase sequence consisting of any one of SEQ ID NOs: 1089, 1757, 141, 1982, 330, 1665, 408, 830, and 899. In certain embodiments, the compound is ION 975616, 994284, 975605, 994282, 975613, 975617, 975735, 975736, or 975612. In any of the foregoing embodiments, the compound can be single-stranded or double-stranded. In any of the foregoing embodiments, the compound can be an antisense compound or oligomeric compound. In certain embodiments, the compound is administered to the individual parenterally. In certain embodiments, administering the compound improves, preserves, or prevents liver damage, steatosis, liver fibrosis, liver inflammation, liver scarring or cirrhosis, liver failure, liver enlargement, elevated transaminases, or hepatic fat accumulation. In certain embodiments, the individual is identified as having, or at risk of having, a disease associated with PNPLA3.

In certain embodiments, a method of inhibiting expression of PNPLA3 in an individual having, or at risk of having, a disease associated with PNPLA3 comprises administering to the individual a compound comprising a PNPLA3 specific inhibitor, thereby inhibiting expression of PNPLA3 in the individual. In certain embodiments, administering the compound inhibits expression of PNPLA3 in the liver. In certain embodiments, the disease is a liver disease. In certain embodiments, the individual has, or is at risk of having, NAFLD, hepatic steatosis, non-alcoholic steatohepatitis (NASH), liver cirrhosis, hepatocellular carcinoma, alcoholic liver disease, alcoholic steatohepatitis (ASH), HCV hepatitis, chronic hepatitis, hereditary hemochromatosis, or primary sclerosing cholangitis. In certain embodiments, the individual has, or is at risk of having, liver damage, steatosis, liver fibrosis, liver inflammation, liver scarring or cirrhosis, liver failure, liver enlargement, elevated transaminases, or hepatic fat accumulation. In certain embodiments, the compound comprises an antisense compound targeted to PNPLA3. In certain embodiments, the compound comprises an oligonucleotide targeted to PNPLA3. In certain embodiments, a compound comprises a modified oligonucleotide 12 to 30 linked nucleosides in length and having a nucleobase sequence comprising at least 8 contiguous nucleobases of any of the nucleobase sequences of SEQ ID NOs: 17-2169. In certain embodiments, a compound comprises a modified oligonucleotide 12 to 30 linked nucleosides in length and having a nucleobase sequence comprising the nucleobase sequence of any one of SEQ ID NOs: 17-2169. In certain embodiments, a compound comprises a modified oligonucleotide consisting of the nucleobase sequence of any one of SEQ ID NOs: 17-2169. In certain embodiments, a compound comprises a modified oligonucleotide 16 to 30 linked nucleosides in length having a nucleobase sequence comprising any one of SEQ ID NOs: 1089, 1757, 141, 1982, 330, 1665, 408, 830, and 899. In certain embodiments, a compound comprises a modified oligonucleotide having a nucleobase sequence consisting of any one of SEQ ID NOs: 1089, 1757, 141, 1982, 330, 1665, 408, 830, and 899. In certain embodiments, the compound is ION 975616, 994284, 975605, 994282, 975613, 975617, 975735, 975736, or 975612. In any of the foregoing embodiments, the compound can be single-stranded or double-stranded. In any of the foregoing embodiments, the compound can be an antisense compound or oligomeric compound. In certain embodiments, the compound is administered to the individual parenterally. In certain embodiments, administering the compound improves, preserves, or prevents liver damage, steatosis, liver fibrosis, liver inflammation, liver scarring or cirrhosis, liver failure, liver enlargement, elevated transaminases, or hepatic fat accumulation.

In certain embodiments, a method of inhibiting expression of PNPLA3 in a cell comprises contacting the cell with a compound comprising a PNPLA3 specific inhibitor, thereby inhibiting expression of PNPLA3 in the cell. In certain embodiments, the cell is a hepatocyte. In certain embodiments, the cell is in the liver. In certain embodiments, the cell is in the liver of an individual who has, or is at risk of having, liver damage, steatosis, liver fibrosis, liver inflammation, liver scarring or cirrhosis, liver failure, liver enlargement, elevated transaminases, or hepatic fat accumulation. In certain embodiments, the compound comprises an antisense compound targeted to PNPLA3. In certain embodiments, the compound comprises an oligonucleotide targeted to PNPLA3. In certain embodiments, a compound comprises a modified oligonucleotide 12 to 30 linked nucleosides in length and having a nucleobase sequence comprising at least 8 contiguous nucleobases of any of the nucleobase sequences of SEQ ID NOs: 17-2169. In certain embodiments, a compound comprises a modified oligonucleotide 12 to 30 linked nucleosides in length and having a nucleobase sequence comprising the nucleobase sequence of any one of SEQ ID NOs: 17-2169. In certain embodiments, a compound comprises a modified oligonucleotide consisting of the nucleobase sequence of any one of SEQ ID NOs: 17-2169. In certain embodiments, a compound comprises a modified oligonucleotide 16 to 30 linked nucleosides in length having a nucleobase sequence comprising any one of SEQ ID NOs: 1089, 1757, 141, 1982, 330, 1665, 408, 830, and 899. In certain embodiments, a compound comprises a modified oligonucleotide having a nucleobase sequence consisting of any one of SEQ ID NOs: 1089, 1757, 141, 1982, 330, 1665, 408, 830, and 899. In certain embodiments, the compound is ION 975616, 994284, 975605, 994282, 975613, 975617, 975735, 975736, or 975612. In any of the foregoing embodiments, the compound can be single-stranded or double-stranded. In any of the foregoing embodiments, the compound can be an antisense compound or oligomeric compound.

In certain embodiments, a method of reducing or inhibiting liver damage, steatosis, liver fibrosis, liver inflammation, liver scarring or cirrhosis, liver failure, liver enlargement, elevated transaminases, or hepatic fat accumulation in an individual having, or at risk of having, a disease associated with PNPLA3 comprises administering to the individual a compound comprising a PNPLA3 specific inhibitor, thereby reducing or inhibiting liver damage, steatosis, liver fibrosis, liver inflammation, liver scarring or cirrhosis, liver failure, liver enlargement, elevated transaminases, or hepatic fat accumulation in the individual. In certain embodiments, the individual has, or is at risk of having, NAFLD, hepatic steatosis, non-alcoholic steatohepatitis (NASH), liver cirrhosis, hepatocellular carcinoma, alcoholic liver disease, alcoholic steatohepatitis (ASH), HCV hepatitis, chronic hepatitis, hereditary hemochromatosis, or primary sclerosing cholangitis. In certain embodiments, the compound comprises an antisense compound targeted to PNPLA3. In certain embodiments, the compound comprises an oligonucleotide targeted to PNPLA3. In certain embodiments, a compound comprises a modified oligonucleotide 12 to 30 linked nucleosides in length and having a nucleobase sequence comprising at least 8 contiguous nucleobases of any of the nucleobase sequences of SEQ ID NOs: 17-2169. In certain embodiments, a compound comprises a modified oligonucleotide 12 to 30 linked nucleosides in length and having a nucleobase sequence comprising the nucleobase sequence of any one of SEQ ID NOs: 17-2169. In certain embodiments, a compound comprises a modified oligonucleotide consisting of the nucleobase sequence of any one of SEQ ID NOs: 17-2169. In certain embodiments, a compound comprises a modified oligonucleotide 16 to 30 linked nucleosides in length having a nucleobase sequence comprising any one of SEQ ID NOs: 1089, 1757, 141, 1982, 330, 1665, 408, 830, and 899. In certain embodiments, a compound comprises a modified oligonucleotide having a nucleobase sequence consisting of any one of SEQ ID NOs: 1089, 1757, 141, 1982, 330, 1665, 408, 830, and 899. In certain embodiments, the compound is ION 975616, 994284, 975605, 994282, 975613, 975617, 975735, 975736, or 975612. In any of the foregoing embodiments, the compound can be single-stranded or double-stranded. In any of the foregoing embodiments, the compound can be an antisense compound or oligomeric compound. In certain embodiments, the compound is administered to the individual parenterally. In certain embodiments, the individual is identified as having, or at risk of having, a disease associated with PNPLA3.

Certain embodiments are drawn to a compound comprising a PNPLA3 specific inhibitor for use in treating a disease associated with PNPLA3. In certain embodiments, the disease is NAFLD, hepatic steatosis, non-alcoholic steatohepatitis (NASH), liver cirrhosis, hepatocellular carcinoma, alcoholic liver disease, alcoholic steatohepatitis (ASH), HCV hepatitis, chronic hepatitis, hereditary hemochromatosis, or primary sclerosing cholangitis. In certain embodiments, the compound comprises an antisense compound targeted to PNPLA3. In certain embodiments, the compound comprises an oligonucleotide targeted to PNPLA3. In certain embodiments, a compound comprises a modified oligonucleotide 12 to 30 linked nucleosides in length and having a nucleobase sequence comprising at least 8 contiguous nucleobases of any of the nucleobase sequences of SEQ ID NOs: 17-2169. In certain embodiments, a compound comprises a modified oligonucleotide 12 to 30 linked nucleosides in length and having a nucleobase sequence comprising the nucleobase sequence of any one of SEQ ID NOs: 17-2169. In certain embodiments, a compound comprises a modified oligonucleotide consisting of the nucleobase sequence of any one of SEQ ID NOs: 17-2169. In certain embodiments, a compound comprises a modified oligonucleotide 16 to 30 linked nucleosides in length having a nucleobase sequence comprising any one of SEQ ID NOs: 1089, 1757, 141, 1982, 330, 1665, 408, 830, and 899. In certain embodiments, a compound comprises a modified oligonucleotide having a nucleobase sequence consisting of any one of SEQ ID NOs: 1089, 1757, 141, 1982, 330, 1665, 408, 830, and 899. In certain embodiments, the compound is ION 975616, 994284, 975605, 994282, 975613, 975617, 975735, 975736, or 975612. In any of the foregoing embodiments, the compound can be single-stranded or double-stranded. In any of the foregoing embodiments, the compound can be an antisense compound or oligomeric compound. In certain embodiments, the compound is administered to the individual parenterally.

Certain embodiments are drawn to a compound comprising a PNPLA3 specific inhibitor for use in reducing or inhibiting liver damage, steatosis, liver fibrosis, liver inflammation, liver scarring or cirrhosis, liver failure, liver enlargement, elevated transaminases, or hepatic fat accumulation in an individual having, or at risk of having, NAFLD, hepatic steatosis, non-alcoholic steatohepatitis (NASH), liver cirrhosis, hepatocellular carcinoma, alcoholic liver disease, alcoholic steatohepatitis (ASH), HCV hepatitis, chronic hepatitis, hereditary hemochromatosis, or primary sclerosing cholangitis. In certain embodiments, the compound comprises an antisense compound targeted to PNPLA3. In certain embodiments, the compound comprises an oligonucleotide targeted to PNPLA3. In certain embodiments, a compound comprises a modified oligonucleotide 12 to 30 linked nucleosides in length and having a nucleobase sequence comprising at least 8 contiguous nucleobases of any of the nucleobase sequences of SEQ ID NOs: 17-2169. In certain embodiments, a compound comprises a modified oligonucleotide 12 to 30 linked nucleosides in length and having a nucleobase sequence comprising the nucleobase sequence of any one of SEQ ID NOs: 17-2169. In certain embodiments, a compound comprises a modified oligonucleotide consisting of the nucleobase sequence of any one of SEQ ID NOs: 17-2169. In certain embodiments, a compound comprises a modified oligonucleotide 16 to 30 linked nucleosides in length having a nucleobase sequence comprising any one of SEQ ID NOs: 1089, 1757, 141, 1982, 330, 1665, 408, 830, and 899. In certain embodiments, a compound comprises a modified oligonucleotide having a nucleobase sequence consisting of any one of SEQ ID NOs: 1089, 1757, 141, 1982, 330, 1665, 408, 830, and 899. In certain embodiments, the compound is ION 975616, 994284, 975605, 994282, 975613, 975617, 975735, 975736, or 975612. In any of the foregoing embodiments, the compound can be single-stranded or double-stranded. In any of the foregoing embodiments, the compound can be an antisense compound or oligomeric compound.

Certain embodiments are drawn to the use of a compound comprising a PNPLA3 specific inhibitor for the manufacture or preparation of a medicament for treating a disease associated with PNPLA3. Certain embodiments are drawn to the use of a compound comprising a PNPLA3 specific inhibitor for the preparation of a medicament for treating a disease associated with PNPLA3. In certain embodiments, the disease is a liver disease. In certain embodiments, the disease is NAFLD, hepatic steatosis, non-alcoholic steatohepatitis (NASH), liver cirrhosis, hepatocellular carcinoma, alcoholic liver disease, alcoholic steatohepatitis (ASH), HCV hepatitis, chronic hepatitis, hereditary hemochromatosis, or primary sclerosing cholangitis. In certain embodiments, the compound comprises an antisense compound targeted to PNPLA3. In certain embodiments, the compound comprises an oligonucleotide targeted to PNPLA3. In certain embodiments, a compound comprises a modified oligonucleotide 12 to 30 linked nucleosides in length and having a nucleobase sequence comprising at least 8 contiguous nucleobases of any of the nucleobase sequences of SEQ ID NOs: 17-2169. In certain embodiments, a compound comprises a modified oligonucleotide 12 to 30 linked nucleosides in length and having a nucleobase sequence comprising the nucleobase sequence of any one of SEQ ID NOs: 17-2169. In certain embodiments, a compound comprises a modified oligonucleotide consisting of the nucleobase sequence of any one of SEQ ID NOs: 17-2169. In certain embodiments, a compound comprises a modified oligonucleotide 16 to 30 linked nucleosides in length having a nucleobase sequence comprising any one of SEQ ID NOs: 1089, 1757, 141, 1982, 330, 1665, 408, 830, and 899. In certain embodiments, a compound comprises a modified oligonucleotide having a nucleobase sequence consisting of any one of SEQ ID NOs: 1089, 1757, 141, 1982, 330, 1665, 408, 830, and 899. In certain embodiments, the compound is ION 975616, 994284, 975605, 994282, 975613, 975617, 975735, 975736, or 975612. In any of the foregoing embodiments, the compound can be single-stranded or double-stranded. In any of the foregoing embodiments, the compound can be an antisense compound or an oligomeric compound.

Certain embodiments are drawn to the use of a compound comprising a PNPLA3 specific inhibitor for the manufacture or preparation of a medicament for reducing or inhibiting liver damage, steatosis, liver fibrosis, liver inflammation, liver scarring or cirrhosis, liver failure, liver enlargement, elevated transaminases, or hepatic fat accumulation in an individual having, or at risk of having, a liver disease associated with PNPLA3. In certain embodiments, the liver disease is NAFLD, hepatic steatosis, non-alcoholic steatohepatitis (NASH), liver cirrhosis, hepatocellular carcinoma, alcoholic liver disease, alcoholic steatohepatitis (ASH), HCV hepatitis, chronic hepatitis, hereditary hemochromatosis, or primary sclerosing cholangitis. Certain embodiments are drawn to use of a compound comprising a PNPLA3 specific inhibitor for the preparation of a medicament for treating a disease associated with PNPLA3. In certain embodiments, the disease is NAFLD, hepatic steatosis, non-alcoholic steatohepatitis (NASH), liver cirrhosis, hepatocellular carcinoma, alcoholic liver disease, alcoholic steatohepatitis (ASH), HCV hepatitis, chronic hepatitis, hereditary hemochromatosis, or primary sclerosing cholangitis. In certain embodiments, the compound comprises an antisense compound targeted to PNPLA3. In certain embodiments, the compound comprises an oligonucleotide targeted to PNPLA3. In certain embodiments, a compound comprises a modified oligonucleotide 12 to 30 linked nucleosides in length and having a nucleobase sequence comprising at least 8 contiguous nucleobases of any of the nucleobase sequences of SEQ ID NOs: 17-2169. In certain embodiments, a compound comprises a modified oligonucleotide 12 to 30 linked nucleosides in length and having a nucleobase sequence comprising the nucleobase sequence of any one of SEQ ID NOs: 17-2169. In certain embodiments, a compound comprises a modified oligonucleotide consisting of the nucleobase sequence of any one of SEQ ID NOs: 17-2169. In certain embodiments, a compound comprises a modified oligonucleotide 16 to 30 linked nucleosides in length having a nucleobase sequence comprising any one of SEQ ID NOs: 1089, 1757, 141, 1982, 330, 1665, 408, 830, and 899. In certain embodiments, a compound comprises a modified oligonucleotide having a nucleobase sequence consisting of any one of SEQ ID NOs: 1089, 1757, 141, 1982, 330, 1665, 408, 830, and 899. In certain embodiments, the compound is ION 975616, 994284, 975605, 994282, 975613, 975617, 975735, 975736, or 975612. In any of the foregoing embodiments, the compound can be single-stranded or double-stranded. In any of the foregoing embodiments, the compound can be an antisense compound or an oligomeric compound.

In some embodiments, the compound targeted to PNPLA3, in an individual having an I148M mutation, comprises a modified oligonucleotide, wherein the modified oligonucleotide has a nucleobase sequence at least 90% identical to SEQ ID NO: 115. In some embodiments, the compound targeted to PNPLA3, in an individual having an I148M mutation, comprises a modified oligonucleotide, wherein the modified oligonucleotide has a nucleobase sequence at least 90% identical to any one of SEQ ID NOs: 2170-2172.

In some embodiments, the compound targeted to PNPLA3, in an individual having an I148M mutation, consists of the sequence of SEQ ID NO: 115, wherein the modified oligonucleotide comprises:
a gap segment consisting of ten linked deoxynucleosides;
a 5′ wing segment consisting of three linked nucleosides; and
a 3′ wing segment consisting of three linked nucleosides;
   wherein the gap segment is positioned between the 5′ wing segment and the 3′ wing segment, wherein each nucleoside of each wing segment comprises a cEt sugar; wherein each internucleoside linkage is a phosphorothioate linkage; and wherein each cytosine is a 5-methylcytosine. In some embodiments, the compound targeted to PNPLA3, in an individual having an 1148M mutation, further comprises a conjugate group, wherein the conjugate group is positioned at the 5′end of the modified oligonucleotide and is

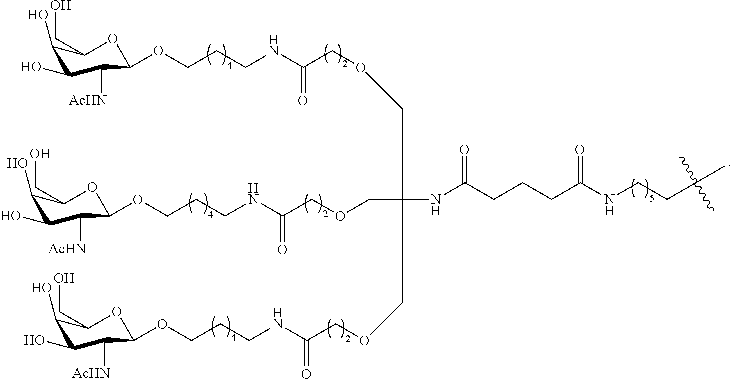

### Certain PNPLA3 Variants

Further embodiments of the present disclosure relate to individuals with certain variants in patatin-like phospholipase domain-containing protein 3 (PNPLA3). An isoleucine-to-methionine mutation at position 148 of the PNPLA3 protein (referred to herein as “PNPLA3 I148M,” “I148M,” “148I allelic variant,” or “PNPLA3 rs738409 polymorphism”; amino acid residue numbering relative to human PNPLA3) can be a strong genetic determinant of non-alcoholic steatohepatitis (NASH). The PNPLA3 I148M mutant protein exhibits reduced enzymatic activity. It was discovered that certain treatments, e.g., compounds described herein, can have an unexpectedly effective treatment of liver disease in individuals, e.g., human patients, with the PNPLA3 I148M mutation. As used herein, an individual “having” or “with” an I148M mutation in PNPLA3 means that the individual has a mutation in the nucleotide sequence of the gene encoding PNPLA3 corresponding to an isoleucine-to-methionine substitution at position 148 of the PNPLA3 protein.

In some embodiments, the present disclosure provides a method of treating an individual having or at risk of having liver disease, comprising administering a compound targeted to PNPLA3 in the individual, wherein the individual has an I148M mutation in PNPLA3.

In some embodiments, treating an individual having liver disease means slowing or stopping the progression of the disease. In some embodiments, treating an individual having liver disease means the liver of the individual returns to a normal, healthy state from a diseased state, for example, as measured by the amount of liver lipids and/or scar tissue, and/or by the amount of liver function as compared to a healthy individual. In some embodiments, when an individual with the PNPLA3 I148M mutation and having liver disease is treated with the method, the individual's liver lipids do not substantially increase. In some embodiments, the method reduces the individual's liver lipids by about 20%, about 25%, about 30%, about 35%, about 40%, about 45%, about 50%, about 55%, about 60%, about 65%, about 70%, about 75%, about 80%, about 85%, about 90%, about 95%, about 99%, or about 100%. Methods of determining the amount of liver lipid are known to one of ordinary skill in the art and include, e.g., Oil Red O staining of a liver biopsy, magnetic resonance spectroscopy (MRS), and lipoprotein subfraction assays.

In some embodiments, when an individual with the PNPLA3 I148M mutation and having liver disease is treated with the method, the individual's liver scar tissue does not substantially increase. Methods of determining the amount of liver scar tissue are known to one of ordinary skill in the art. In some embodiments, the method reduces the individual's liver scar tissue by about 20%, about 25%, about 30%, about 35%, about 40%, about 45%, about 50%, about 55%, about 60%, about 65%, about 70%, about 75%, about 80%, about 85%, about 90%, about 95%, about 99%, or about 100%. Methods of determining the amount of scar tissue are known to one of ordinary skill in the art and include, for example, imaging tests such as, e.g., ultrasonography, computed tomography (CT), magnetic resonance imaging (MRI), ultrasound elastography, magnetic resonance elastography, and/or acoustic radiation force impulse imaging; blood tests; and liver biopsy.

In some embodiments, when an individual with the PNPLA3 I148M mutation and having liver disease is treated with the method, the individual's liver function does not substantially decrease. In some embodiments, after being treated with the method, the individual's liver function increases. In some embodiments, after being treated with the method, the individual's liver function increases by about 20%, about 25%, about 30%, about 35%, about 40%, about 45%, about 50%, about 55%, about 60%, about 65%, about 70%, about 75%, about 80%, about 85%, about 90%, about 95%, about 99%, or about 100%. In some embodiments, after being treated with the method, the individual's liver function is about 50%, about 60%, about 70%, about 80%, about 90%, about 95%, about 99%, or greater than 99% of the liver function of a healthy individual. Methods of measuring liver function are known to one of ordinary skill in the art and include, for example, measuring levels of one or more of alanine transaminase (ALT), aspartate aminotransferase (AST), alkaline phosphatase (ALP), albumin, and bilirubin.

In some embodiments, treating an individual at risk of having liver disease means preventing or reducing the likelihood that the individual develops the disease, for example, by reducing liver lipids and/or scar tissue, or any other compounds, e.g., proteins, polynucleotides, that can cause or exacerbate the development of liver disease.

Examples of liver diseases including, e.g., diseases associated with PNPLA3, are described herein. In some embodiments, the liver disease is selected from non-alcoholic fatty liver disease (NAFLD), hepatic steatosis, non-alcoholic steatohepatitis (NASH), liver cirrhosis, hepatocellular carcinoma, alcoholic liver disease, alcoholic steatohepatitis (ASH), HCV hepatitis, chronic hepatitis, hereditary hemochromatosis, or primary sclerosing cholangitis. In some embodiments, the liver disease is hepatic steatosis. In some embodiments, the method provides a highly effective treatment for the liver disease, e.g., hepatic steatosis, when administered to an individual having an I148M mutation in PNPLA3.

In some embodiments, the present disclosure provides a method of reducing one or more of liver damage, hepatic steatosis, liver inflammation, liver fibrosis, and hepatic lipogenesis in an individual, comprising administering a compound targeted to PNPLA3 to the individual, wherein the individual has an I148M mutation in PNPLA3. In some embodiments, the method reduces or inhibits liver inflammation. In some embodiments, the method reduces or inhibits liver fibrosis.

In some embodiments, treating liver disease in an individual comprises reducing one or more of liver damage, hepatic steatosis, liver inflammation, liver fibrosis, and hepatic lipogenesis. Examples of liver diseases are described herein. In some embodiments, the method is highly effective at reducing one or more of liver damage, hepatic steatosis, liver inflammation, liver fibrosis, and hepatic lipogenesis in an individual having an I148M mutation in PNPLA3. In some embodiments, the method is highly effective at reducing one or more of hepatic steatosis, liver inflammation, and liver fibrosis in an individual having an I148M mutation in PNPLA3.

In some embodiments, reducing or inhibiting liver inflammation comprises reducing liver macrophage levels. Liver macrophage levels can be quantified, e.g., by immunohistochemical staining of macrophage antigen 2 (Mac2), which is expressed on the surface of inflammatory macrophages. In some embodiments, the liver macrophage levels are reduced by at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% relative to an individual not administered the compound targeted to PNPLA3, as measured by an immunohistochemical staining, e.g., of Mac2, of a liver section of the individual.

In some embodiments, the reducing liver macrophage levels comprises reducing the amount of monocyte chemoattractant protein (MCP1) in the liver, e.g., in a liver cell. MCP1, also known as chemokine (C-C-motif) ligand 2 (CCL2) and small inducible cytokine A2, and its receptor C-C chemokine receptor-2 (CCR2) play a role in recruiting monocytes, dendritic cells, and macrophages to sites of inflammation in the liver. In some embodiments, reducing expression of MCP1 in the liver reduces liver macrophage levels. In some embodiments, reducing expression of MCP1 in the liver reduces liver inflammation. In some embodiments, reducing liver macrophage levels comprises reducing the amount of haptoglobin in the liver, e.g., in a liver cell. Haptoglobin is an acute phase protein produced in the liver and adipose tissue, typically in response to inflammation, infection, and/or tissue injury. Haptoglobin can attract monocytes and macrophages in part through interaction with CCR2, described herein. In some embodiments, reducing expression of haptoglobin in the liver reduces liver inflammation.

In some embodiments, the present disclosure provides a method of reducing protein levels of one or more of haptoglobin, MCP1, and TIMP2 in an individual, comprising administering a compound targeted to PNPLA3 to the individual, wherein the individual has an I148M mutation in PNPLA3.

Haptoglobin and its role in liver inflammation are described herein. In some embodiments, the method reduces protein levels of haptoglobin in an individual with an I148M mutation in PNPLA3. In some embodiments, the method reduces expression of haptoglobin in an individual with an I148M mutation in PNPLA3. In some embodiments, protein levels of haptoglobin are reduced by at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% relative to an individual not administered the compound targeted to PNPLA3. Methods of measuring haptoglobin levels in a sample are known to one of ordinary skill in the art and can include, e.g., spectrophotometry, immunoassay, electrophoresis and the like. In some embodiments, haptoglobin levels in a sample from an individual, e.g., an individual with PNPLA3 I148M, are measured by a turbidimetric assay, e.g., using an ABX Pentra instrument. In some embodiments, haptoglobin protein levels in a sample from an individual, e.g., an individual with PNPLA3 I148M, are measured by a colorimetric assay, e.g., the PHASE™ Range Haptoglobin Colorimetric Assay.

MCP1 and its role in liver inflammation are described herein. In some embodiments, the method reduces protein levels of MCP1 in an individual with an I148M mutation in PNPLA3. In some embodiments, the method reduces expression of MCP1 in an individual with an I148M mutation in PNPLA3. In some embodiments, protein levels of MCP1 are reduced by at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% relative to an individual not administered the compound targeted to PNPLA3. Methods of measuring MCP1 levels in a sample are known to one of ordinary skill in the art and can include, e.g., immunoassay (e.g., ELISA), immunoblotting, electrophoresis, chromatography and the like. In some embodiments, MCP1 protein levels in a sample from an individual, e.g., an individual with PNPLA I148M, are measured by immunoblotting of a liver sample of the individual.

In some embodiments, the method reduces protein levels of TIMP2 in an individual with an I148M mutation in PNPLA3. In some embodiments, the method reduces expression of TIMP2 in an individual with an I148M mutation in PNPLA3. Tissue inhibitor of metalloproteases 2 (TIMP2) is a member of the TIMP family, which are generally natural inhibitors of the matrix metalloproteinase (MMP) group of peptidases involved in degradation of the extracellular matrix. TIMP2 expression was shown to be elevated in activated human hepatic stellate cells and in fibrotic rat livers (see, e.g., Xu et al., Gut 54(1):142-151, 2005; and Peng et al., Exp Biol Med 238(6):668-677, 2013). Moreover, TIMP2 can inhibit the collagenolytic activity of matrix metalloproteinase 2 (MMP2), which is increased in experimental models of liver fibrosis and in humans with chronic liver disease (see, e.g., Linden et al., Mol Metab 22:49-61, 2019). In some embodiments, TIMP2 inhibition of MMP2 reduces liver fibrosis. In some embodiments, protein levels of TIMP2 are reduced by at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% relative to an individual not administered the compound targeted to PNPLA3. Methods of measuring TIMP2 levels in a sample are known to one of ordinary skill in the art and can include, e.g., immunoassay (e.g., ELISA), immunoblotting, electrophoresis, chromatography and the like. In some embodiments, TIMP2 protein levels in a sample from an individual, e.g., an individual with PNPLA I148M, are measured by immunoblotting of a liver sample of the individual.

In some embodiments, the individual has a heterozygous I148M mutation in PNPLA3. As used herein, a “heterozygous” mutation means a mutation of one allele (with the other allele being non-mutated, i.e., wild type). In some embodiments, the individual has a homozygous I148M mutation in PNPLA3. As used herein, a “homozygous” mutation means an identical mutation of two alleles. In some embodiments, the individual has a compound heterozygous mutation at position 148 of PNPLA3. As used herein, a “compound heterozygous” mutation means a different mutation at each of the two alleles. For example, a compound heterozygous mutation at position 148 of PNPLA3 can comprise an I148M mutation at one allele, and a different mutation at the other allele. In some embodiments, the compound heterozygous mutation at position 148 of PNPLA3, wherein one allele is I148M, has the same phenotype as the homozygous I148M mutation in PNPLA3. In some embodiments, the compound heterozygous mutation at position 148 of PNPLA3, wherein one allele is I148M, has a different phenotype from either the homozygous I148M mutation or the heterozygous I148M mutation in PNPLA3. In some embodiments, an individual having a PNPLA3 I148M mutation in at least one allele has elevated risk of liver disease. In some embodiments, an individual having a homozygous PNPLA3 I148M mutation has an elevated risk of liver disease. In some embodiments, the methods provided herein unexpectedly provide a highly effective treatment of liver disease in an individual having a PNPLA3 I148M mutation in at least one allele. In some embodiments, the methods provided herein unexpectedly provide a highly effective treatment of liver disease in an individual having a homozygous PNPLA3 I148M mutation.

In some embodiments, the individual is a human individual. In some embodiments, the individual is an animal, e.g., a cow, horse, dog, cat, rat, or mouse. In embodiments where the individual is non-human, it will be understood by one of ordinary skill in the art that the amino acid residue numbers for PNPLA3 may not be the same as the human PNPLA3. The skilled artisan can determine the residue corresponding to residue 148 in human PNPLA3 using sequence alignment methods known in the field, e.g., BLAST, Clustal, HMMER, and the like.

In some embodiments, the methods herein comprise administering a compound targeted to PNPLA3 to an individual having an I148M mutation in PNPLA3. In some embodiments, the compound targeted to PNPLA3, in an individual having an T148M mutation, is an antisense compound targeted to PNPLA3. Antisense compounds are described herein. In some embodiments, the antisense compound targeted to PNPLA3, in an individual having an I148M mutation, is a short interfering RNA (siRNA). In some embodiments, the antisense compound targeted to PNPLA3, in an individual having an I148M mutation, comprises any of

(SEQ ID NO: 2170)
5′-GGUCCUCUCAGAUCUUGUGtt-3′,
(SEQ ID NO: 2171)
5′-GGAGUGAGUGACAACGUACtt-3′,
or
(SEQ ID NO: 2172)
5′-GGUUCUUGGAAGAGAAGGGtt-3′

In some embodiments, the compound targeted to PNPLA3, in an individual having an I148M mutation, comprises a modified oligonucleotide, wherein the modified oligonucleotide has a nucleobase sequence about 80%, about 85%, about 90%, about 91%, about 92%, about 93%, about 94%, about 95%, about 96%, about 97%, about 98%, about 99%, or about 100% identical to any of SEQ ID NOs: 2170-2172.

In some embodiments, the compound targeted to PNPLA3, in an individual having an I148M mutation, is an antisense oligonucleotide (ASO). ASOs are described herein. In some embodiments, the compound targeted to PNPLA3, in an individual having an I148M mutation, comprises a modified oligonucleotide 8 to 80 linked nucleosides in length having a having a nucleobase sequence comprising at least 8, at least 9, at least 10, at least 11, or at least 12 contiguous nucleobases of any of the nucleobase sequences of SEQ ID NOs: 17-2169. In some embodiments, the compound comprises a modified oligonucleotide 12 to 30 linked nucleosides in length and having a nucleobase sequence comprising at least 8 contiguous nucleobases of any of the nucleobase sequences of SEQ ID NOs: 17-2169. In some embodiments, the compound comprises a modified oligonucleotide 12 to 30 linked nucleosides in length and having a nucleobase sequence comprising the nucleobase sequence of any one of SEQ ID NOs: 17-2169. In some embodiments, the compound comprises a modified oligonucleotide consisting of the nucleobase sequence of any one of SEQ ID NOs: 17-2169. In certain embodiments, the compound comprises a modified oligonucleotide 16 to 30 linked nucleosides in length having a nucleobase sequence comprising any one of SEQ ID NOs: 1089, 1757, 141, 1982, 330, 1665, 408, 830, and 899. In some embodiments, the compound comprises a modified oligonucleotide having a nucleobase sequence consisting of any one of SEQ ID NOs: 1089, 1757, 141, 1982, 330, 1665, 408, 830, and 899. In some embodiments, the compound is ION 975616, 994284, 975605, 994282, 975613, 975617, 975735, 975736, or 975612. In some embodiments, the compound comprises a modified oligonucleotide, wherein the modified oligonucleotide has a nucleobase sequence about 80%, about 85%, about 90%, about 91%, about 92%, about 93%, about 94%, about 95%, about 96%, about 97%, about 98%, about 99%, or about 100% identical to SEQ ID NO: 115.

In some embodiments, the compound comprises a modified oligonucleotide 8 to 80 linked nucleosides in length, wherein the modified oligonucleotide has a nucleobase sequence comprising a portion of at least 8 contiguous nucleobases 100% complementary to an equal length portion of nucleobases 5567-5642, 5644-5731, 5567-5731, 5567-5620, 13697-13733, 20553-20676, 20664-20824, 20553-20824, or 25844-25912 of SEQ ID NO: 2, and wherein the nucleobase sequence of the modified oligonucleotide is at least 85%, at least 90%, at least 95%, or 100% complementary to SEQ ID NO: 2. In some embodiments, the compound comprises a modified oligonucleotide 8 to 80 linked nucleosides in length, wherein the modified oligonucleotide has a nucleobase sequence complementary within nucleobases 5567-5642, 5644-5731, 5567-5731, 5567-5620, 13697-13733, 20553-20676, 20664-20824, 20553-20824, or 25844-25912 of SEQ ID NO: 2, and wherein said modified oligonucleotide is at least 85%, at least 90%, at least 95%, or 100% complementary to SEQ ID NO: 2.

In some embodiments, the compound comprises a modified oligonucleotide 8 to 80 linked nucleosides in length, wherein the modified oligonucleotide has a nucleobase sequence comprising a portion of at least 8 contiguous nucleobases complementary to an equal length portion of nucleobases 5567-5642, 5644-5731, 5567-5731, 5567-5620, 13697-13733, 20553-20676, 20664-20824, 20553-20824, or 25844-25912 of a PNPLA3 nucleic acid having the nucleobase sequence of SEQ ID NO: 2, wherein the nucleobase sequence of the modified oligonucleotide is complementary to SEQ ID NO: 2. In some embodiments, the compound comprises a modified oligonucleotide 8 to 80 linked nucleosides in length, wherein the modified oligonucleotide has a nucleobase sequence comprising a 16 nucleobase portion complementary to an equal length portion of nucleobases 5567-5642, 5644-5731, 5567-5731, 5567-5620, 13697-13733, 20553-20676, 20664-20824, 20553-20824, or 25844-25912 of SEQ ID NO: 2. In some embodiments, the modified oligonucleotide has a nucleobase sequence at least 80%, at least 85%, at least 90%, at least 95% or 100% complementary to SEQ ID NO: 2 over the entire length of the nucleobase sequence.

In any of the foregoing methods or uses, the compound can be targeted to PNPLA3. In certain embodiments, the compound comprises or consists of a modified oligonucleotide, for example, a modified oligonucleotide 8 to 80 linked nucleosides in length, 10 to 30 linked nucleosides in length, 12 to 30 linked nucleosides in length, or 20 linked nucleosides in length. In certain embodiments, the modified oligonucleotide is at least 80%, at least 85%, at least 90%, at least 95% or 100% complementary to any of the nucleobase sequences recited in SEQ ID NOs: 1-10. In certain embodiments, the modified oligonucleotide comprises at least one modified internucleoside linkage, at least one modified sugar and/or at least one modified nucleobase.

In certain embodiments, the modified internucleoside linkage is a phosphorothioate internucleoside linkage, the modified sugar is a bicyclic sugar or a 2′-O-methoxyethyl modified sugar, and the modified nucleobase is a 5-methylcytosine. In certain embodiments, the modified oligonucleotide comprises a gap segment consisting of linked deoxynucleosides; a 5′ wing segment consisting of linked nucleosides; and a 3′ wing segment consisting of linked nucleosides, wherein the gap segment is positioned immediately adjacent to and between the 5′ wing segment and the 3′ wing segment and wherein each nucleoside of each wing segment comprises a modified sugar.

In any of the foregoing embodiments, the modified oligonucleotide is 12 to 30, 15 to 30, 15 to 25, 15 to 24, 16 to 24, 17 to 24, 18 to 24, 19 to 24, 20 to 24, 19 to 22, 20 to 22, 16 to 20, or 16 or 20 linked nucleosides in length. In certain embodiments, the modified oligonucleotide is at least 80%, at least 85%, at least 90%, at least 95% or 100% complementary to any of the nucleobase sequences recited in SEQ ID NOs: 1-10.

In any of the foregoing methods or uses, the compound comprises or consists of a modified oligonucleotide 16 to 30 linked nucleosides in length and having a nucleobase sequence comprising any one of SEQ ID NOs: 17-2169, wherein the modified oligonucleotide comprises:
a gap segment consisting of linked 2′-deoxynucleosides;
a 5′ wing segment consisting of linked nucleosides; and
a 3′ wing segment consisting of linked nucleosides;
   wherein the gap segment is positioned between the 5′ wing segment and the 3′ wing segment and wherein each nucleoside of each wing segment comprises a modified sugar.

In any of the foregoing methods or uses, the compound comprises or consists a modified oligonucleotide 16 linked nucleosides in length having a nucleobase sequence comprising the sequence recited in any one of SEQ ID NOs: 1089, 1757, 141, 1982, 330, 1665, 408, 830, and 899, wherein the modified oligonucleotide comprises
a gap segment consisting of ten linked deoxynucleosides;
a 5′ wing segment consisting of three linked nucleosides; and
a 3′ wing segment consisting of three linked nucleosides;
   wherein the gap segment is positioned between the 5′ wing segment and the 3′ wing segment, wherein each nucleoside of each wing segment comprises a cEt sugar; wherein each internucleoside linkage is a phosphorothioate linkage and wherein each cytosine is a 5-methylcytosine. In certain embodiments, the modified oligonucleotide is 16-30 linked nucleosides in length.

In some embodiments, the compound targeted to PNPLA3, in an individual having an I148M mutation, comprises a modified oligonucleotide, wherein the modified oligonucleotide has a nucleobase sequence at least 90% identical to SEQ ID NO: 115. In some embodiments, the compound targeted to PNPLA3, in an individual having an I148M mutation, comprises a modified oligonucleotide, wherein the modified oligonucleotide has a nucleobase sequence at least 90% identical to any one of SEQ ID NOs: 2170-2172.

In some embodiments, the compound targeted to PNPLA3, in an individual having an I148M mutation, consists of the sequence of SEQ ID NO: 115, wherein the modified oligonucleotide comprises:
a gap segment consisting of ten linked deoxynucleosides;
a 5′ wing segment consisting of three linked nucleosides; and
a 3′ wing segment consisting of three linked nucleosides;

wherein the gap segment is positioned between the 5′ wing segment and the 3′ wing segment, wherein each nucleoside of each wing segment comprises a cEt sugar; wherein each internucleoside linkage is a phosphorothioate linkage; and wherein each cytosine is a 5-methylcytosine. In some embodiments, the compound targeted to PNPLA3, in an individual having an 1148M mutation, further comprises a conjugate group, wherein the conjugate group is positioned at the 5′end of the modified oligonucleotide and is

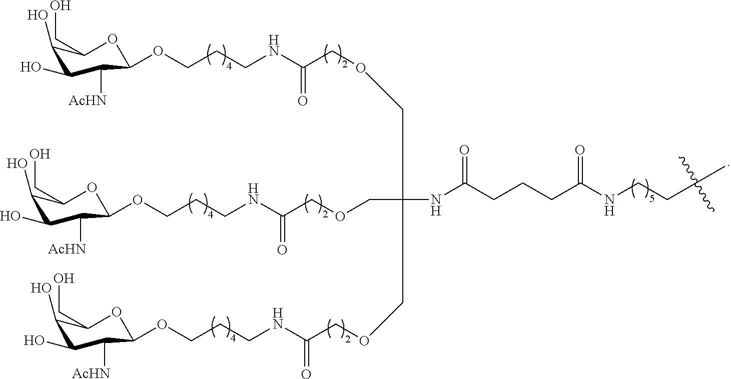

In certain embodiments, a compound comprises or consists of ION 916333 or salt thereof, having the

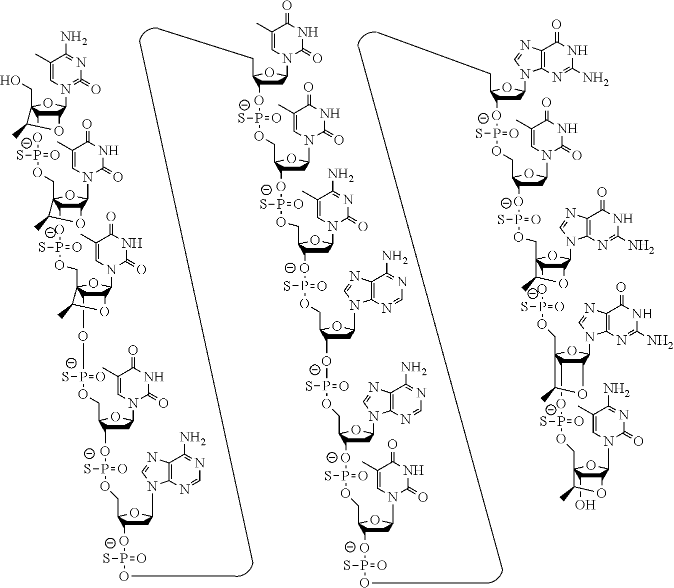

In certain embodiments, a compound comprises or consists of ION 975616 or salt thereof, having the

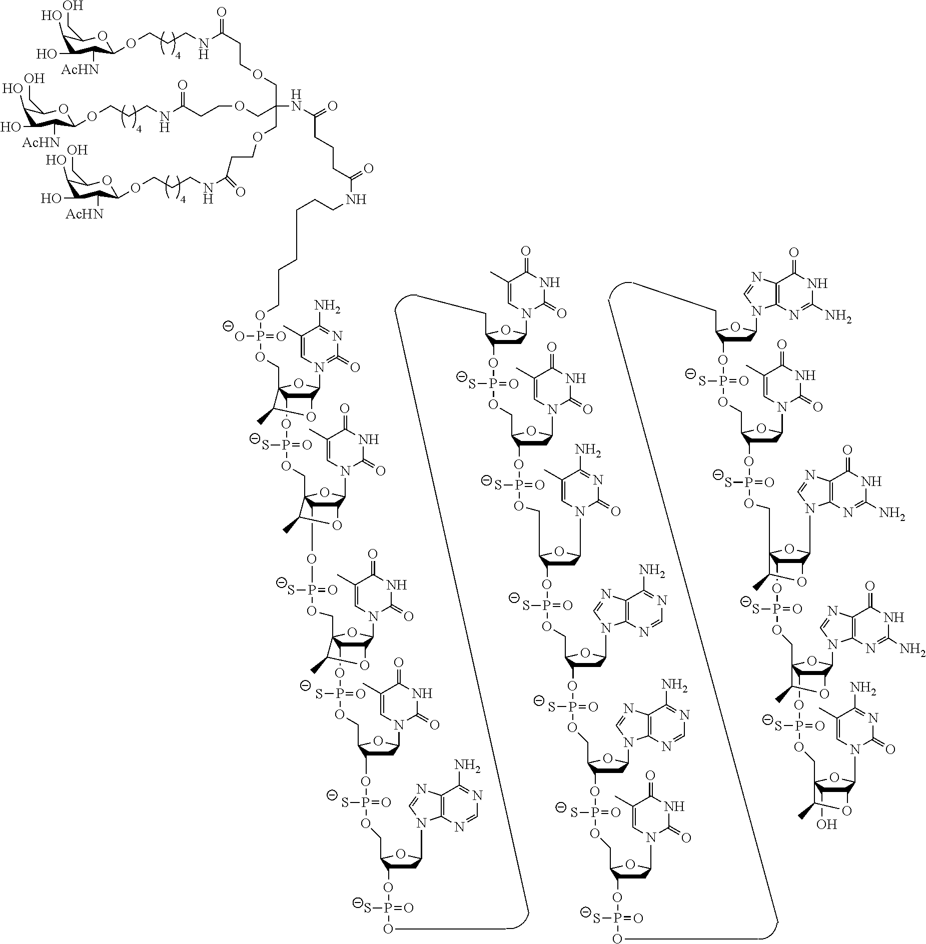

In certain embodiments, a compound comprises or consists of the sodium salt of ION 975616, having the following chemical structure:

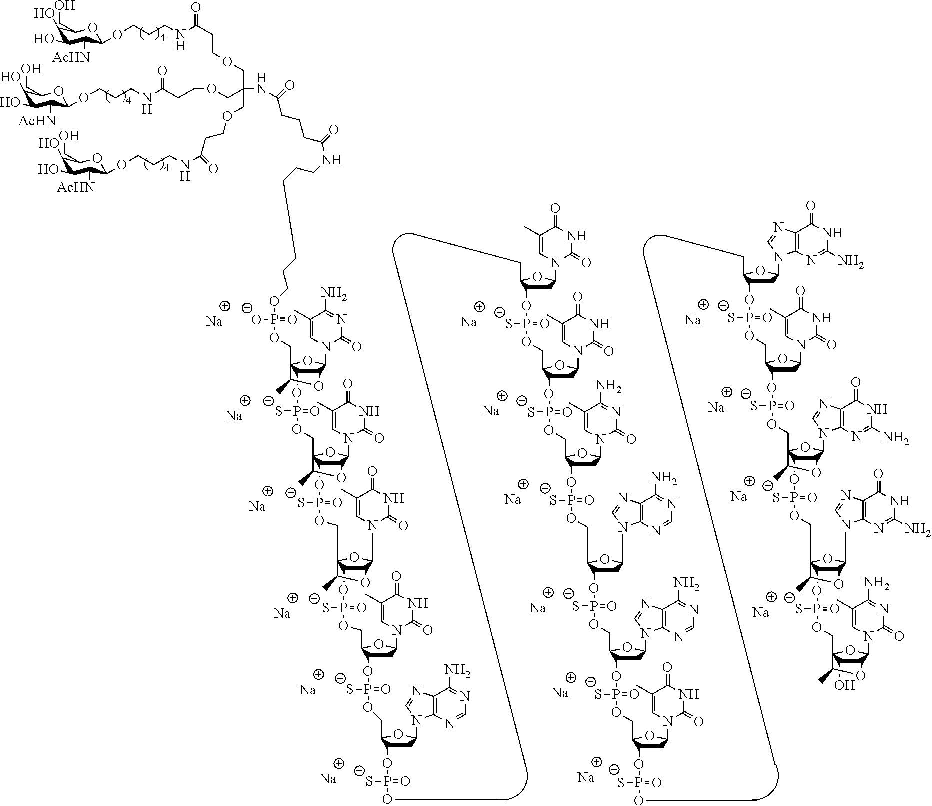

In certain embodiments, a compound comprises or consists of ION 975613 or salt thereof, having the

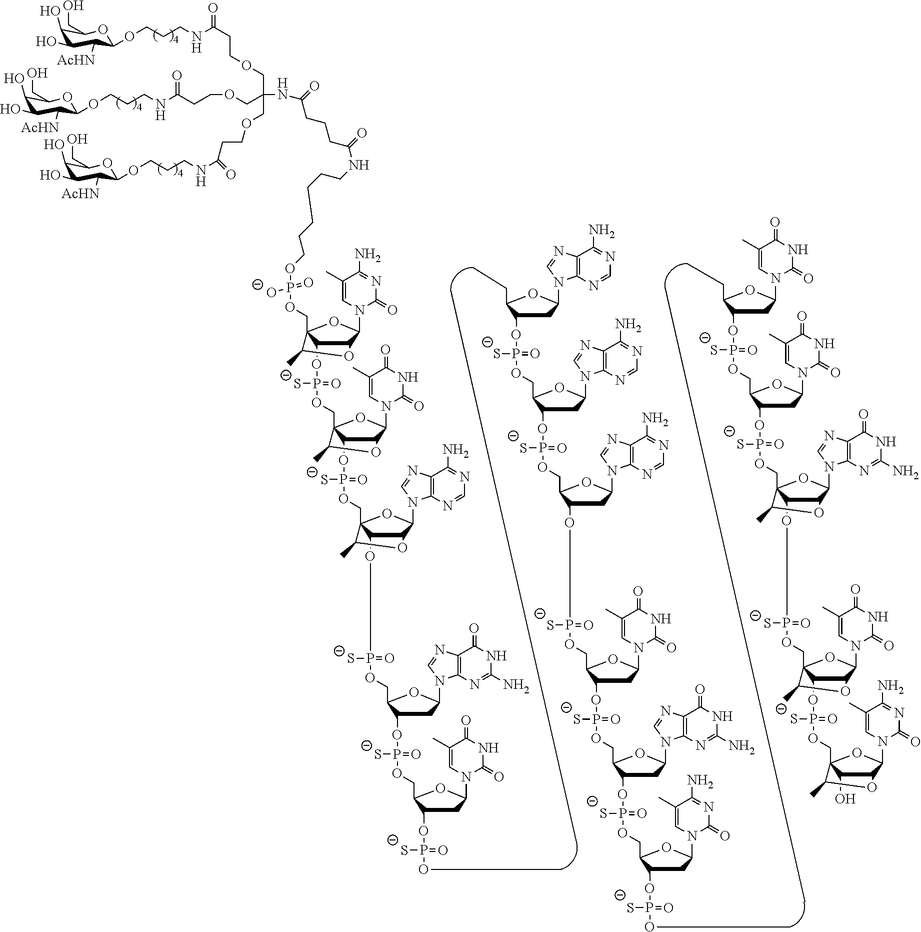

In certain embodiments, a compound comprises or consists of the sodium salt of ION 975613, having the following chemical structure:

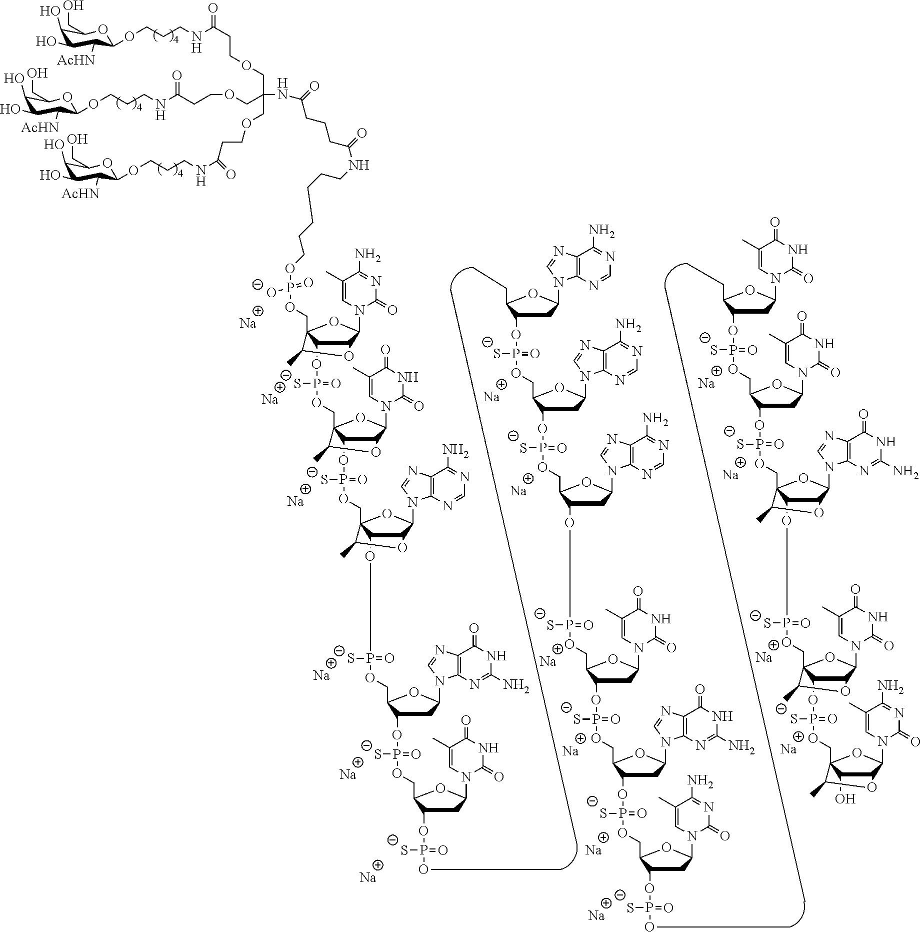

In certain embodiments, a compound comprises or consists of ION 975612 or salt thereof, having the

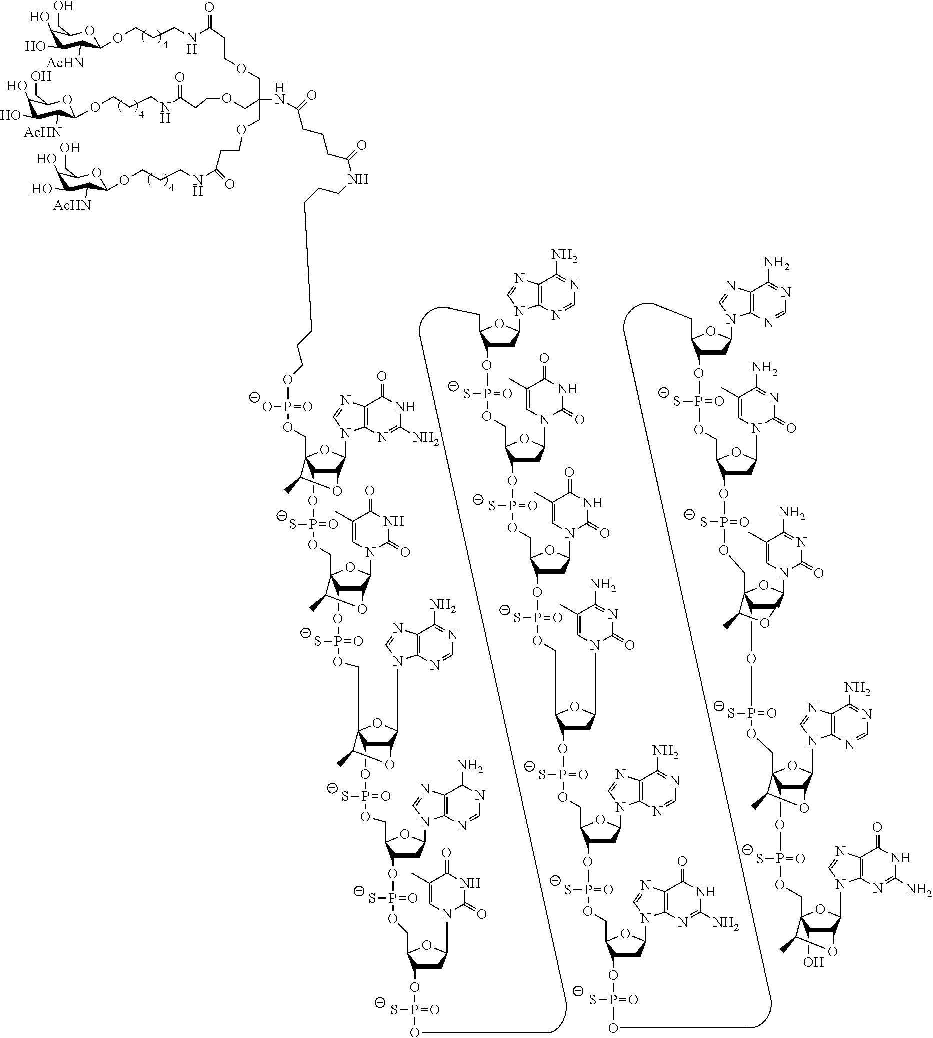

In certain embodiments, a compound comprises or consists of the sodium salt of ION 975612, having

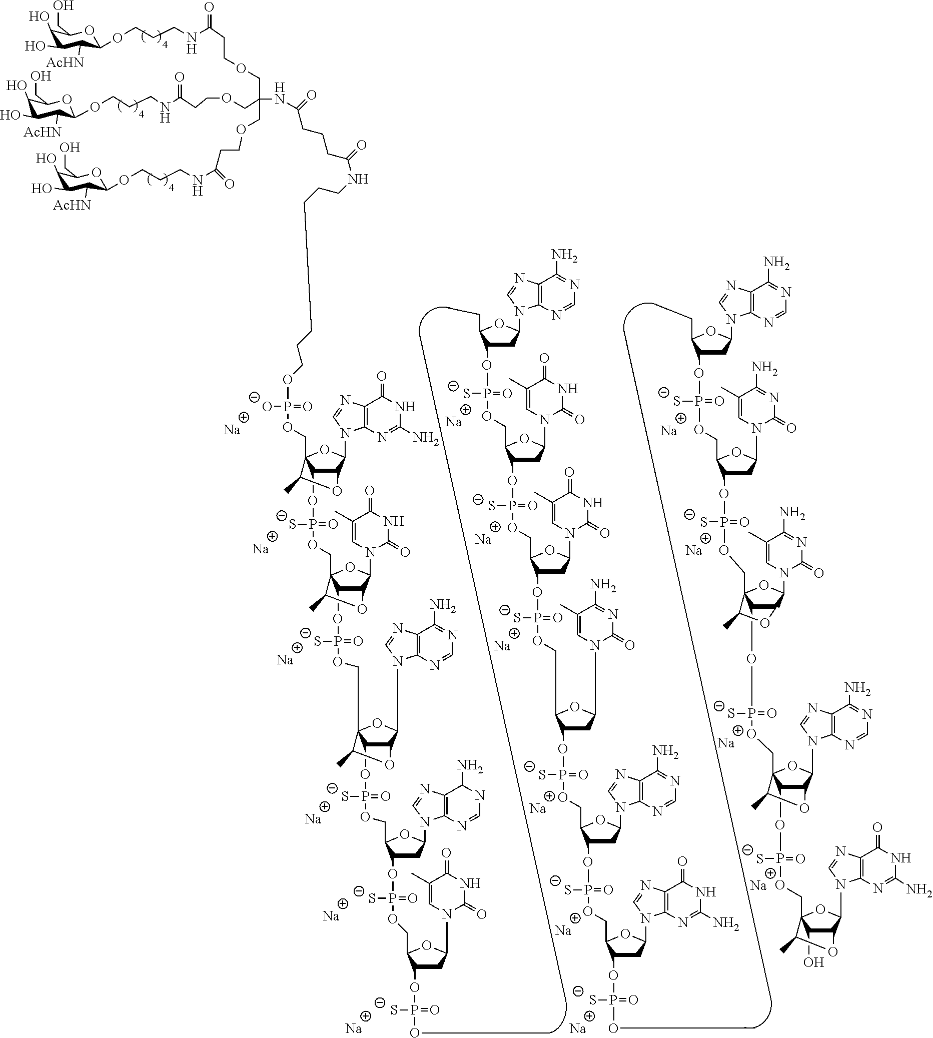

In certain embodiments, a compound comprises or consists of ION 916789 or salt thereof, having the

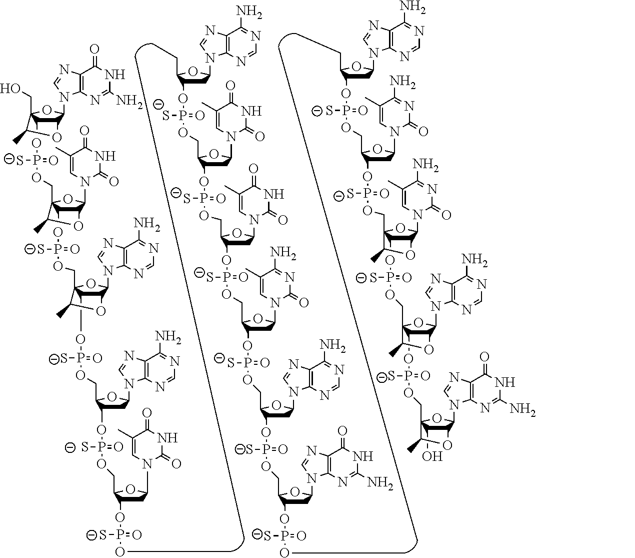

In certain embodiments, a compound comprises or consists of the sodium salt of ION 916789, having the following chemical structure:

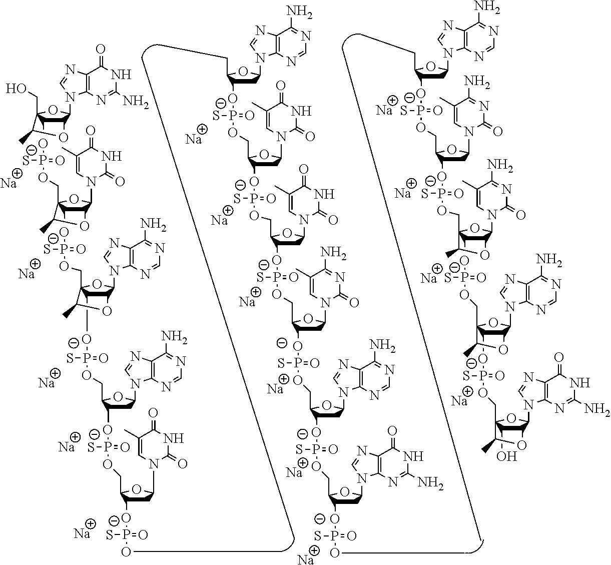

In certain embodiments, a compound comprises or consists of ION 916602 or salt thereof, having the

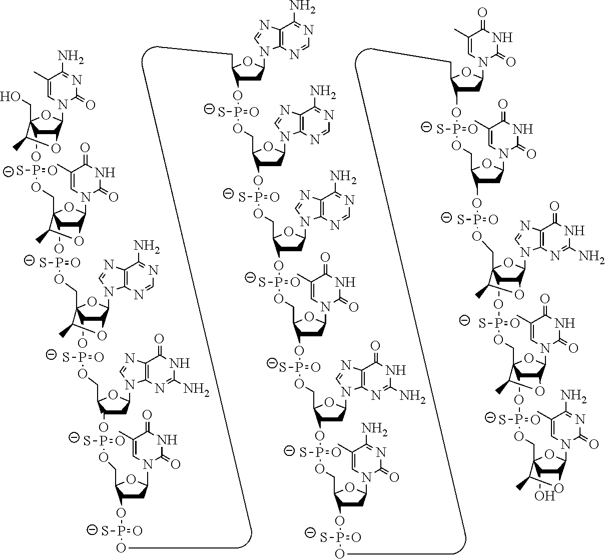

In certain embodiments, a compound comprises or consists of the sodium salt of ION 916602, having the following chemical structure:

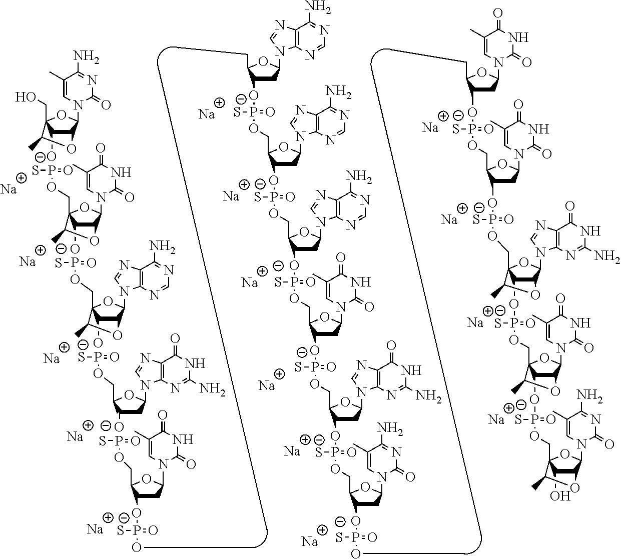

In any of the foregoing methods or uses, the compound can be administered parenterally. For example, in certain embodiments the compound can be administered through injection or infusion. Parenteral administration includes subcutaneous administration, intravenous administration, intramuscular administration, intraarterial administration, intraperitoneal administration, or intracranial administration, e.g. intrathecal or intracerebroventricular administration.

### Certain Compounds

In certain embodiments, compounds described herein can be antisense compounds. In certain embodiments, the antisense compound comprises or consists of an oligomeric compound. In certain embodiments, the oligomeric compound comprises a modified oligonucleotide. In certain embodiments, the modified oligonucleotide has a nucleobase sequence complementary to that of a target nucleic acid.

In certain embodiments, a compound described herein comprises or consists of a modified oligonucleotide. In certain embodiments, the modified oligonucleotide has a nucleobase sequence complementary to that of a target nucleic acid.

In certain embodiments, a compound or antisense compound is single-stranded. Such a single-stranded compound or antisense compound comprises or consists of an oligomeric compound. In certain embodiments, such an oligomeric compound comprises or consists of an oligonucleotide and optionally a conjugate group. In certain embodiments, the oligonucleotide is an antisense oligonucleotide. In certain embodiments, the oligonucleotide is modified. In certain embodiments, the oligonucleotide of a single-stranded antisense compound or oligomeric compound comprises a self-complementary nucleobase sequence.

In certain embodiments, compounds are double-stranded. Such double-stranded compounds comprise a first modified oligonucleotide having a region complementary to a target nucleic acid and a second modified oligonucleotide having a region complementary to the first modified oligonucleotide. In certain embodiments, the modified oligonucleotide is an RNA oligonucleotide. In such embodiments, the thymine nucleobase in the modified oligonucleotide is replaced by a uracil nucleobase. In certain embodiments, compound comprises a conjugate group. In certain embodiments, one of the modified oligonucleotides is conjugated. In certain embodiments, both the modified oligonucleotides are conjugated. In certain embodiments, the first modified oligonucleotide is conjugated. In certain embodiments, the second modified oligonucleotide is conjugated. In certain embodiments, the first modified oligonucleotide is 16-30 linked nucleosides in length and the second modified oligonucleotide is 16-30 linked nucleosides in length. In certain embodiments, one of the modified oligonucleotides has a nucleobase sequence comprising at least 8 contiguous nucleobases of any of SEQ ID NOs: 17-2169.

In certain embodiments, antisense compounds are double-stranded. Such double-stranded antisense compounds comprise a first oligomeric compound having a region complementary to a target nucleic acid and a second oligomeric compound having a region complementary to the first oligomeric compound. The first oligomeric compound of such double stranded antisense compounds typically comprises or consists of a modified oligonucleotide and optionally a conjugate group. The oligonucleotide of the second oligomeric compound of such a double-stranded antisense compound may be modified or unmodified. Either or both oligomeric compounds of a double-stranded antisense compound may comprise a conjugate group. The oligomeric compounds of double-stranded antisense compounds may include non-complementary overhanging nucleosides.

Examples of single-stranded and double-stranded compounds include, but are not limited to, oligonucleotides, siRNAs, microRNA targeting oligonucleotides, and single-stranded RNAi compounds, such as small hairpin RNAs (shRNAs), single-stranded siRNAs (ssRNAs), and microRNA mimics.

In certain embodiments, a compound described herein has a nucleobase sequence that, when written in the 5′ to 3′ direction, comprises the reverse complement of the target segment of a target nucleic acid to which it is targeted.

In certain embodiments, a compound described herein comprises an oligonucleotide 12 to 30 linked subunits in length. In certain embodiments, a compound described herein comprises an oligonucleotide 12 to 22 linked subunits in length. In certain embodiments, compound described herein comprises an oligonucleotide 14 to 30 linked subunits in length. In certain embodiments, compound described herein comprises an oligonucleotide 14 to 20 linked subunits in length. In certain embodiments, a compound described herein comprises an oligonucleotide 15 to 30 linked subunits in length. In certain embodiments, a compound described herein comprises an oligonucleotide 15 to 20 linked subunits in length. In certain embodiments, a compound described herein comprises an oligonucleotide 16 to 30 linked subunits in length. In certain embodiments, a compound described herein comprises an oligonucleotide 16 to 20 linked subunits in length. In certain embodiments, a compound described herein comprises an oligonucleotide 17 to 30 linked subunits in length.

In certain embodiments, a compound described herein comprises an oligonucleotide 17 to 20 linked subunits in length. In certain embodiments, a compound described herein comprises an oligonucleotide 18 to 30 linked subunits in length. In certain embodiments, a compound described herein comprises an oligonucleotide 18 to 20 linked subunits in length. In certain embodiments, a compound described herein comprises an oligonucleotide 20 to 30 linked subunits in length. In other words, such oligonucleotides are 12 to 30 linked subunits, 14 to 30 linked subunits, 14 to 20 subunits, 15 to 30 subunits, 15 to 20 subunits, 16 to 30 subunits, 16 to 20 subunits, 17 to 30 subunits, 17 to 20 subunits, 18 to 30 subunits, 18 to 20 subunits, or 20 to 30 subunits in length, respectively. In certain embodiments, a compound described herein comprises an oligonucleotide 14 linked subunits in length. In certain embodiments, a compound described herein comprises an oligonucleotide 16 linked subunits in length. In certain embodiments, a compound described herein comprises an oligonucleotide 17 linked subunits in length. In certain embodiments, compound described herein comprises an oligonucleotide 18 linked subunits in length. In certain embodiments, a compound described herein comprises an oligonucleotide 19 linked subunits in length. In certain embodiments, a compound described herein comprises an oligonucleotide 20 linked subunits in length. In other embodiments, a compound described herein comprises an oligonucleotide 8 to 80, 12 to 50, 13 to 30, 13 to 50, 14 to 30, 14 to 50, 15 to 30, 15 to 50, 16 to 30, 16 to 50, 17 to 30, 17 to 50, 18 to 22, 18 to 24, 18 to 30,18 to 50, 19 to 22, 19 to 30, 19 to 50, or 20 to 30 linked subunits. In certain such embodiments, the compound described herein comprises an oligonucleotide 8, 9, 10, 11, 12, 13, 14, 15, 16, 17, 18, 19, 20, 21, 22, 23, 24, 25, 26, 27, 28, 29, or 30 linked subunits in length, or a range defined by any two of the above values. In some embodiments the linked subunits are nucleotides, nucleosides, or nucleobases.

In certain embodiments, the compound may further comprise additional features or elements, such as a conjugate group, that are attached to the oligonucleotide. In certain embodiments, such compounds are antisense compounds. In certain embodiments, such compounds are oligomeric compounds. In embodiments where a conjugate group comprises a nucleoside (i.e. a nucleoside that links the conjugate group to the oligonucleotide), the nucleoside of the conjugate group is not counted in the length of the oligonucleotide.

In certain embodiments, compounds may be shortened or truncated. For example, a single subunit may be deleted from the 5′ end (5′ truncation), or alternatively from the 3′ end (3′ truncation). A shortened or truncated compound targeted to a PNPLA3 nucleic acid may have two subunits deleted from the 5′ end, or alternatively, may have two subunits deleted from the 3′ end of the compound. Alternatively, the deleted nucleosides may be dispersed throughout the compound.

When a single additional subunit is present in a lengthened compound, the additional subunit may be located at the 5′ or 3′ end of the compound. When two or more additional subunits are present, the added subunits may be adjacent to each other, for example, in a compound having two subunits added to the 5′ end (5′ addition), or alternatively, to the 3′ end (3′ addition) of the compound. Alternatively, the added subunits may be dispersed throughout the compound.

It is possible to increase or decrease the length of a compound, such as an oligonucleotide, and/or introduce mismatch bases without eliminating activity (Woolf et al. Proc. Natl. Acad. Sci. USA 1992, 89:7305-7309; Gautschi et al. J. Natd. Cancer Inst. March 2001, 93:463-471; Maher and Dolnick Nuc. Acid. Res. 1998, 16:3341-3358). However, seemingly small changes in oligonucleotide sequence, chemistry and motif can make large differences in one or more of the many properties required for clinical development (Seth et al. J. Med. Chem. 2009, 52, 10; Egli et al. J. Am. Chem. Soc. 2011, 133, 16642).

In certain embodiments, compounds described herein are interfering RNA compounds (RNAi), which include double-stranded RNA compounds (also referred to as short-interfering RNA or siRNA) and single-stranded RNAi compounds (or ssRNA). Such compounds work at least in part through the RISC pathway to degrade and/or sequester a target nucleic acid (thus, include microRNA/microRNA-mimic compounds). As used herein, the term siRNA is meant to be equivalent to other terms used to describe nucleic acid molecules that are capable of mediating sequence-specific RNAi, for example, short interfering RNA (siRNA), double-stranded RNA (dsRNA), micro-RNA (miRNA), short hairpin RNA (shRNA), short interfering oligonucleotide, short interfering nucleic acid, short interfering modified oligonucleotide, chemically modified siRNA, post-transcriptional gene silencing RNA (ptgsRNA), and others. In addition, as used herein, the term “RNAi” is meant to be equivalent to other terms used to describe sequence-specific RNA interference, such as post transcriptional gene silencing, translational inhibition, or epigenetics.

In certain embodiments, a compound described herein can comprise any of the oligonucleotide sequences targeted to PNPLA3 described herein. In certain embodiments, the compound can be double-stranded. In certain embodiments, the compound comprises a first strand comprising at least an 8, 9, 10, 11, 12, 13, 14, 15, or 16 contiguous nucleobase portion of any one of SEQ ID NOs: 17-2169 and a second strand.

In certain embodiments, the compound comprises a first strand comprising the nucleobase sequence of any one of SEQ ID NOs: 17-2169 and a second strand. In certain embodiments, the compound comprises ribonucleotides in which the first strand has uracil (U) in place of thymine (T) in any one of SEQ ID NOs: 17-2169. In certain embodiments, the compound comprises (i) a first strand comprising a nucleobase sequence complementary to the site on PNPLA3 to which any of SEQ ID NOs: 17-2169 is targeted, and (ii) a second strand. In certain embodiments, the compound comprises one or more modified nucleotides in which the 2′ position of the sugar contains a halogen (such as fluorine group; 2′-F) or contains an alkoxy group (such as a methoxy group; 2′-OMe). In certain embodiments, the compound comprises at least one 2′-F sugar modification and at least one 2′-OMe sugar modification. In certain embodiments, the at least one 2′-F sugar modification and at least one 2′-OMe sugar modification are arranged in an alternating pattern for at least 2, 3, 4, 5, 6, 7, 8, 9, 10, 11, 12, 13, 14, 15, 16, 17, 18, 19, or 20 contiguous nucleobases along a strand of the dsRNA compound. In certain embodiments, the compound comprises one or more linkages between adjacent nucleotides other than a naturally-occurring phosphodiester linkage. Examples of such linkages include phosphoramide, phosphorothioate, and phosphorodithioate linkages. The compounds may also be chemically modified nucleic acid molecules as taught in U.S. Pat. No. 6,673,661. In other embodiments, the compound contains one or two capped strands, as disclosed, for example, by WO 00/63364, filed Apr. 19, 2000.

In certain embodiments, the first strand of the compound is an siRNA guide strand and the second strand of the compound is an siRNA passenger strand. In certain embodiments, the second strand of the compound is complementary to the first strand. In certain embodiments, each strand of the compound is 16, 17, 18, 19, 20, 21, 22, or 23 linked nucleosides in length. In certain embodiments, the first or second strand of the compound can comprise a conjugate group.

In certain embodiments, a compound described herein can comprise any of the oligonucleotide sequences targeted to PNPLA3 described herein. In certain embodiments, the compound is single stranded. In certain embodiments, such a compound is a single-stranded RNAi (ssRNAi) compound. In certain embodiments, the compound comprises at least an 8, 9, 10, 11, 12, 13, 14, 15, or 16 contiguous nucleobase portion of any one of SEQ ID NOs: 17-2169. In certain embodiments, the compound comprises the nucleobase sequence of any one of SEQ ID NOs: 17-2169. In certain embodiments, the compound comprises ribonucleotides in which uracil (U) is in place of thymine (T) in any one of SEQ ID NOs: 17-2169. In certain embodiments, the compound comprises a nucleobase sequence complementary to the site on PNPLA3 to which any of SEQ ID NOs: 17-2169 is targeted. In certain embodiments, the compound comprises one or more modified nucleotides in which the 2′ position in the sugar contains a halogen (such as fluorine group; 2′-F) or contains an alkoxy group (such as a methoxy group; 2′-OMe). In certain embodiments, the compound comprises at least one 2′-F sugar modification and at least one 2′-OMe sugar modification. In certain embodiments, the at least one 2′-F sugar modification and at least one 2′-OMe sugar modification are arranged in an alternating pattern for at least 2, 3, 4, 5, 6, 7, 8, 9, 10, 11, 12, 13, 14, 15, 16, 17, 18, 19, or 20 contiguous nucleobases along a strand of the compound. In certain embodiments, the compound comprises one or more linkages between adjacent nucleotides other than a naturally-occurring phosphodiester linkage. Examples of such linkages include phosphoramide, phosphorothioate, and phosphorodithioate linkages. The compounds may also be chemically modified nucleic acid molecules as taught in U.S. Pat. No. 6,673,661. In other embodiments, the compound contains a capped strand, as disclosed, for example, by WO 00/63364, filed Apr. 19, 2000. In certain embodiments, the compound consists of 16, 17, 18, 19, 20, 21, 22, or 23 linked nucleosides. In certain embodiments, the compound can comprise a conjugate group.

### Certain Mechanisms

In certain embodiments, compounds described herein comprise or consist of modified oligonucleotides. In certain embodiments, compounds described herein are antisense compounds. In certain embodiments, compounds comprise oligomeric compounds. In certain embodiments, compounds described herein are capable of hybridizing to a target nucleic acid, resulting in at least one antisense activity. In certain embodiments, compounds described herein selectively affect one or more target nucleic acid. Such compounds comprise a nucleobase sequence that hybridizes to one or more target nucleic acid, resulting in one or more desired antisense activity and does not hybridize to one or more non-target nucleic acid or does not hybridize to one or more non-target nucleic acid in such a way that results in a significant undesired antisense activity.

In certain antisense activities, hybridization of a compound described herein to a target nucleic acid results in recruitment of a protein that cleaves the target nucleic acid. For example, certain compounds described herein result in RNase H mediated cleavage of the target nucleic acid. RNase H is a cellular endonuclease that cleaves the RNA strand of an RNA:DNA duplex. The DNA in such an RNA:DNA duplex need not be unmodified DNA. In certain embodiments, compounds described herein are sufficiently “DNA-like” to elicit RNase H activity. Further, in certain embodiments, one or more non-DNA-like nucleoside in the gap of a gapmer is tolerated.

In certain antisense activities, compounds described herein or a portion of the compound is loaded into an RNA-induced silencing complex (RISC), ultimately resulting in cleavage of the target nucleic acid. For example, certain compounds described herein result in cleavage of the target nucleic acid by Argonaute. Compounds that are loaded into RISC are RNAi compounds. RNAi compounds may be double-stranded (siRNA) or single-stranded (ssRNA).

In certain embodiments, hybridization of compounds described herein to a target nucleic acid does not result in recruitment of a protein that cleaves that target nucleic acid. In certain such embodiments, hybridization of the compound to the target nucleic acid results in alteration of splicing of the target nucleic acid. In certain embodiments, hybridization of the compound to a target nucleic acid results in inhibition of a binding interaction between the target nucleic acid and a protein or other nucleic acid. In certain such embodiments, hybridization of the compound to a target nucleic acid results in alteration of translation of the target nucleic acid.

Antisense activities may be observed directly or indirectly. In certain embodiments, observation or detection of an antisense activity involves observation or detection of a change in an amount of a target nucleic acid or protein encoded by such target nucleic acid, a change in the ratio of splice variants of a nucleic acid or protein, and/or a phenotypic change in a cell or animal.

### Target Nucleic Acids, Target Regions and Nucleotide Sequences

In certain embodiments, compounds described herein comprise or consist of an oligonucleotide comprising a region that is complementary to a target nucleic acid. In certain embodiments, the target nucleic acid is an endogenous RNA molecule. In certain embodiments, the target nucleic acid encodes a protein. In certain such embodiments, the target nucleic acid is selected from an mRNA and a pre-mRNA, including intronic, exonic and untranslated regions. In certain embodiments, the target RNA is an mRNA. In certain embodiments, the target nucleic acid is a pre-mRNA. In certain such embodiments, the target region is entirely within an intron. In certain embodiments, the target region spans an intron/exon junction. In certain embodiments, the target region is at least 50% within an intron.

Nucleotide sequences that encode PNPLA3 include, without limitation, the following: RefSeq or GENBANK Accession Nos. NM_025225.2 (incorporated by reference, disclosed herein as SEQ ID NO: 1); GENBANK Accession No. NC_000022.11 truncated from nucleotides 43921001 to U.S. Pat. No. 43,954,500 (incorporated by reference, disclosed herein as SEQ ID NO: 2); AK123806.1(incorporated by reference, disclosed herein as SEQ ID NO: 3); BQ686328.1 (incorporated by reference, disclosed herein as SEQ ID NO: 4); BF762711.1 (incorporated by reference, disclosed herein as SEQ ID NO: 5); DA290491.1 (incorporated by reference, disclosed herein as SEQ ID NO: 6); and the sequences listed as SEQ ID Nos. 7, 8, 9, and 10.

### Hybridization

In some embodiments, hybridization occurs between a compound disclosed herein and a PNPLA3 nucleic acid. The most common mechanism of hybridization involves hydrogen bonding (e.g., Watson-Crick, Hoogsteen or reversed Hoogsteen hydrogen bonding) between complementary nucleobases of the nucleic acid molecules.

Hybridization can occur under varying conditions. Hybridization conditions are sequence-dependent and are determined by the nature and composition of the nucleic acid molecules to be hybridized.

Methods of determining whether a sequence is specifically hybridizable to a target nucleic acid are well known in the art. In certain embodiments, the compounds provided herein are specifically hybridizable with a PNPLA3 nucleic acid.

### Complementarity

An oligonucleotide is said to be complementary to another nucleic acid when the nucleobase sequence of such oligonucleotide or one or more regions thereof matches the nucleobase sequence of another oligonucleotide or nucleic acid or one or more regions thereof when the two nucleobase sequences are aligned in opposing directions. Nucleobase matches or complementary nucleobases, as described herein, are limited to the following pairs: adenine (A) and thymine (T), adenine (A) and uracil (U), cytosine (C) and guanine (G), and 5-methyl cytosine (mC) and guanine (G), unless otherwise specified. Complementary oligonucleotides and/or nucleic acids need not have nucleobase complementarity at each nucleoside and may include one or more nucleobase mismatches. An oligonucleotide is fully complementary or 100% complementary when such oligonucleotides have nucleobase matches at each nucleoside without any nucleobase mismatches.

In certain embodiments, compounds described herein comprise or consist of modified oligonucleotides. In certain embodiments, compounds described herein are antisense compounds. In certain embodiments, compounds comprise oligomeric compounds. Non-complementary nucleobases between a compound and a PNPLA3 nucleic acid may be tolerated provided that the compound remains able to specifically hybridize to a target nucleic acid. Moreover, a compound may hybridize over one or more segments of a PNPLA3 nucleic acid such that intervening or adjacent segments are not involved in the hybridization event (e.g., a loop structure, mismatch or hairpin structure).

In certain embodiments, the compounds provided herein, or a specified portion thereof are at least, or are up to 70%, 80%, 85%, 86%, 87%, 88%, 89%, 90%, 91%, 92%, 93%, 94%, 95%, 96%, 97%, 98%, 99%, or 100% complementary to a PNPLA3 nucleic acid, a target region, target segment, or specified portion thereof. In certain embodiments, the compounds provided herein, or a specified portion thereof, are 70% to 75%, 75% to 80%, 80% to 85%, 85% to 90%, 90% to 95%, 95% to 100%, or any number in between these ranges, complementary to a PNPLA3 nucleic acid, a target region, target segment, or specified portion thereof. Percent complementarity of a compound with a target nucleic acid can be determined using routine methods.

For example, a compound in which 18 of 20 nucleobases of the compound are complementary to a target region, and would therefore specifically hybridize, would represent 90 percent complementarity. In this example, the remaining non-complementary nucleobases may be clustered or interspersed with complementary nucleobases and need not be contiguous to each other or to complementary nucleobases. As such, a compound which is 18 nucleobases in length having four non-complementary nucleobases which are flanked by two regions of complete complementarity with the target nucleic acid would have 77.8% overall complementarity with the target nucleic acid. Percent complementarity of a compound with a region of a target nucleic acid can be determined routinely using BLAST programs (basic local alignment search tools) and PowerBLAST programs known in the art (Altschul et al., J. Mol. Biol., 1990, 215, 403 410; Zhang and Madden, Genome Res., 1997, 7, 649 656). Percent homology, sequence identity or complementarity, can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482 489).

In certain embodiments, compounds described herein, or specified portions thereof, are fully complementary (i.e. 100% complementary) to a target nucleic acid, or specified portion thereof. For example, a compound may be fully complementary to a PNPLA3 nucleic acid, or a target region, or a target segment or target sequence thereof. As used herein, “fully complementary” means each nucleobase of a compound is complementary to the corresponding nucleobase of a target nucleic acid. For example, a 20 nucleobase compound is fully complementary to a target sequence that is 400 nucleobases long, so long as there is a corresponding 20 nucleobase portion of the target nucleic acid that is fully complementary to the compound. “Fully complementary” can also be used in reference to a specified portion of the first and/or the second nucleic acid. For example, a 20 nucleobase portion of a 30 nucleobase compound can be “fully complementary” to a target sequence that is 400 nucleobases long. The 20 nucleobase portion of the 30 nucleobase compound is fully complementary to the target sequence if the target sequence has a corresponding 20 nucleobase portion wherein each nucleobase is complementary to the 20 nucleobase portion of the compound. At the same time, the entire 30 nucleobase compound may or may not be fully complementary to the target sequence, depending on whether the remaining 10 nucleobases of the compound are also complementary to the target sequence.

In certain embodiments, compounds described herein comprise one or more mismatched nucleobases relative to the target nucleic acid. In certain such embodiments, antisense activity against the target is reduced by such mismatch, but activity against a non-target is reduced by a greater amount. Thus, in certain such embodiments, selectivity of the compound is improved. In certain embodiments, the mismatch is specifically positioned within an oligonucleotide having a gapmer motif. In certain such embodiments, the mismatch is at position 1, 2, 3, 4, 5, 6, 7, or 8 from the 5′-end of the gap region. In certain such embodiments, the mismatch is at position 9, 8, 7, 6, 5, 4, 3, 2, 1 from the 3′-end of the gap region. In certain such embodiments, the mismatch is at position 1, 2, 3, or 4 from the 5′-end of the wing region. In certain such embodiments, the mismatch is at position 4, 3, 2, or 1 from the 3′-end of the wing region. In certain embodiments, the mismatch is specifically positioned within an oligonucleotide not having a gapmer motif. In certain such embodiments, the mismatch is at position 1, 2, 3, 4, 5, 6, 7, 8, 9, 10, 11, or 12 from the 5′-end of the oligonucleotide. In certain such embodiments, the mismatch is at position 1, 2, 3, 4, 5, 6, 7, 8, 9, 10, 11, or 12 from the 3′-end of the oligonucleotide.

The location of a non-complementary nucleobase may be at the 5′ end or 3′ end of the compound. Alternatively, the non-complementary nucleobase or nucleobases may be at an internal position of the compound. When two or more non-complementary nucleobases are present, they may be contiguous (i.e. linked) or non-contiguous. In one embodiment, a non-complementary nucleobase is located in the wing segment of a gapmer oligonucleotide.

In certain embodiments, compounds described herein that are, or are up to 11, 12, 13, 14, 15, 16, 17, 18, 19, or 20 nucleobases in length comprise no more than 4, no more than 3, no more than 2, or no more than 1 non-complementary nucleobase(s) relative to a target nucleic acid, such as a PNPLA3 nucleic acid, or specified portion thereof.

In certain embodiments, compounds described herein that are, or are up to 11, 12, 13, 14, 15, 16, 17, 18, 19, 20, 21, 22, 23, 24, 25, 26, 27, 28, 29, or 30 nucleobases in length comprise no more than 6, no more than 5, no more than 4, no more than 3, no more than 2, or no more than 1 non-complementary nucleobase(s) relative to a target nucleic acid, such as a PNPLA3 nucleic acid, or specified portion thereof.

In certain embodiments, compounds described herein also include those which are complementary to a portion of a target nucleic acid. As used herein, “portion” refers to a defined number of contiguous (i.e. linked) nucleobases within a region or segment of a target nucleic acid. A “portion” can also refer to a defined number of contiguous nucleobases of a compound. In certain embodiments, the compounds, are complementary to at least an 8 nucleobase portion of a target segment. In certain embodiments, the compounds are complementary to at least a 9 nucleobase portion of a target segment. In certain embodiments, the compounds are complementary to at least a 10 nucleobase portion of a target segment. In certain embodiments, the compounds are complementary to at least an 11 nucleobase portion of a target segment. In certain embodiments, the compounds are complementary to at least a 12 nucleobase portion of a target segment. In certain embodiments, the compounds are complementary to at least a 13 nucleobase portion of a target segment. In certain embodiments, the compounds are complementary to at least a 14 nucleobase portion of a target segment. In certain embodiments, the compounds are complementary to at least a 15 nucleobase portion of a target segment. In certain embodiments, the compounds are complementary to at least a 16 nucleobase portion of a target segment. Also contemplated are compounds that are complementary to at least a 9, 10, 17, 18, 19, 20, or more nucleobase portion of a target segment, or a range defined by any two of these values.

### Identity

The compounds provided herein may also have a defined percent identity to a particular nucleotide sequence, SEQ ID NO, or compound represented by a specific ION number, or portion thereof. In certain embodiments, compounds described herein are antisense compounds or oligomeric compounds. In certain embodiments, compounds described herein are modified oligonucleotides. As used herein, a compound is identical to the sequence disclosed herein if it has the same nucleobase pairing ability. For example, a RNA which contains uracil in place of thymidine in a disclosed DNA sequence would be considered identical to the DNA sequence since both uracil and thymidine pair with adenine. Shortened and lengthened versions of the compounds described herein as well as compounds having non-identical bases relative to the compounds provided herein also are contemplated. The non-identical bases may be adjacent to each other or dispersed throughout the compound. Percent identity of an compound is calculated according to the number of bases that have identical base pairing relative to the sequence to which it is being compared.

In certain embodiments, compounds described herein, or portions thereof, are, or are at least, 70%, 75%, 80%, 85%, 90%, 91%, 92%, 93%, 94%, 95%, 96%, 97%, 98%, 99% or 100% identical to one or more of the compounds or SEQ ID NOs, or a portion thereof, disclosed herein. In certain embodiments, compounds described herein are about 70%, 75%, 80%, 85%, 90%, 91%, 92%, 93%, 94%, 95%, 96%, 97%, 98%, or 99% identical, or any percentage between such values, to a particular nucleotide sequence, SEQ ID NO, or compound represented by a specific ION number, or portion thereof, in which the compounds comprise an oligonucleotide having one or more mismatched nucleobases. In certain such embodiments, the mismatch is at position 1, 2, 3, 4, 5, 6, 7, 8, 9, 10, 11, or 12 from the 5′-end of the oligonucleotide. In certain such embodiments, the mismatch is at position 1, 2, 3, 4, 5, 6, 7, 8, 9, 10, 11, or 12 from the 3′-end of the oligonucleotide.

In certain embodiments, compounds described herein comprise or consist of antisense compounds. In certain embodiments, a portion of the antisense compound is compared to an equal length portion of the target nucleic acid. In certain embodiments, an 8, 9, 10, 11, 12, 13, 14, 15, 16, 17, 18, 19, 20, 21, 22, 23, 24, or 25 nucleobase portion is compared to an equal length portion of the target nucleic acid.

In certain embodiments, compounds described herein comprise or consist of oligonucleotides. In certain embodiments, a portion of the oligonucleotide is compared to an equal length portion of the target nucleic acid. In certain embodiments, an 8, 9, 10, 11, 12, 13, 14, 15, 16, 17, 18, 19, 20, 21, 22, 23, 24, or 25 nucleobase portion is compared to an equal length portion of the target nucleic acid.

### Certain Modified Compounds

In certain embodiments, compounds described herein comprise or consist of oligonucleotides consisting of linked nucleosides. Oligonucleotides may be unmodified oligonucleotides (RNA or DNA) or may be modified oligonucleotides. Modified oligonucleotides comprise at least one modification relative to unmodified RNA or DNA (i.e., comprise at least one modified nucleoside (comprising a modified sugar moiety and/or a modified nucleobase) and/or at least one modified internucleoside linkage).

### Modified Nucleosides

Modified nucleosides comprise a modified sugar moiety or a modified nucleobase or both a modified sugar moiety and a modified nucleobase.

### 1. Modified Sugar Moieties

In certain embodiments, sugar moieties are non-bicyclic modified sugar moieties. In certain embodiments, modified sugar moieties are bicyclic or tricyclic sugar moieties. In certain embodiments, modified sugar moieties are sugar surrogates. Such sugar surrogates may comprise one or more substitutions corresponding to those of other types of modified sugar moieties.

In certain embodiments, modified sugar moieties are non-bicyclic modified furanosyl sugar moieties comprising one or more acyclic substituent, including, but not limited, to substituents at the 2′, 4′, and/or 5′ positions. In certain embodiments, the furanosyl sugar moiety is a ribosyl sugar moiety. In certain embodiments, one or more acyclic substituent of non-bicyclic modified sugar moieties is branched. Examples of 2′-substituent groups suitable for non-bicyclic modified sugar moieties include but are not limited to: 2′-F, 2′—OCH₃ (“OMe” or “O-methyl”), and 2′-O(CH₂)₂OCH₃_(“MOE”). In certain embodiments, 2′-substituent groups are selected from among: halo, allyl, amino, azido, SH, CN, OCN, CF₃, OCF₃, O—C₁-C₁₀ alkoxy, 0-C₁-C₁₀ substituted alkoxy, O—C₁-C₁₀ alkyl, O—C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH₂C(═O)—N(Rₘ)(Rₙ), where each Rₘ and Rₐ is, independently, H, an amino protecting group, or substituted or unsubstituted C₁-C₁₀ alkyl, and the 2′-substituent groups described in Cook et al., U.S. Pat. No. 6,531,584; Cook et al., U.S. Pat. No. 5,859,221; and Cook et al., U.S. Pat. No. 6,005,087.

Certain embodiments of these 2′-substituent groups can be further substituted with one or more substituent groups independently selected from among: hydroxyl, amino, alkoxy, carboxy, benzyl, phenyl, nitro (NO₂), thiol, thioalkoxy, thioalkyl, halogen, alkyl, aryl, alkenyl and alkynyl. Examples of 4′-substituent groups suitable for linearly non-bicyclic modified sugar moieties include, but are not limited to, alkoxy (e.g., methoxy), alkyl, and those described in Manoharan et al., WO 2015/106128. Examples of 5′-substituent groups suitable for non-bicyclic modified sugar moieties include, but are not limited to: 5′-methyl(R or S), 5′-vinyl, and 5′-methoxy. In certain embodiments, non-bicyclic modified sugars comprise more than one non-bridging sugar substituent, for example, 2′-F-5′-methyl sugar moieties and the modified sugar moieties and modified nucleosides described in Migawa et al., WO 2008/101157 and Rajeev et al., US2013/0203836.

In certain embodiments, a 2′-substituted nucleoside or 2′-non-bicyclic modified nucleoside comprises a sugar moiety comprising a linear 2′-substituent group selected from: F, NH₂, N₃, OCF₃, OCH₃, O(CH₂)₃NH₂, CH₂CH═CH₂, OCH₂CH═CH₂, OCH₂CH₂OCH₃, O(CH₂)2SCH₃, O(CH₂)₂ON(Rₘ)(Rₙ), O(CH₂)₂O(CH₂)₂N(CH₃)₂, and N-substituted acetamide (OCH₂C(═O)—N(Rₘ)(Rₙ)), where each Rₘ and Rₐ is, independently, H, an amino protecting group, or substituted or unsubstituted C₁-C₁₀ alkyl.

In certain embodiments, a 2′-substituted nucleoside or 2′-non-bicyclic modified nucleoside comprises a sugar moiety comprising a linear 2′-substituent group selected from: F, OCF₃, OCH₃, OCH₂CH₂OCH₃, O(CH₂)2SCH₃, O(CH₂)20N(CH₃)2, O(CH₂)₂O(CH₂)₂N(CH₃)2, and OCH₂C(═O)—N(H)CH₃ (“NMA”).

In certain embodiments, a 2′-substituted nucleoside or 2′-non-bicyclic modified nucleoside comprises a sugar moiety comprising a linear 2′-substituent group selected from: F, OCH₃, and OCH₂CH₂OCH₃.

Nucleosides comprising modified sugar moieties, such as non-bicyclic modified sugar moieties, are referred to by the position(s) of the substitution(s) on the sugar moiety of the nucleoside. For example, nucleosides comprising 2′-substituted or 2′-modified sugar moieties are referred to as 2′-substituted nucleosides or 2′-modified nucleosides.

Certain modified sugar moieties comprise a bridging sugar substituent that forms a second ring resulting in a bicyclic sugar moiety. In certain such embodiments, the bicyclic sugar moiety comprises a bridge between the 4′ and the 2′ furanose ring atoms. In certain such embodiments, the furanose ring is a ribose ring. Examples of such 4′ to 2′ bridging sugar substituents include, but are not limited to: 4′—CH₂-2′, 4′—(CH₂)₂-2′, 4′—(CH₂)₃-2′, 4′—CH₂—O—2′ (“LNA”), 4′—CH₂—S-2′, 4′—(CH₂)2—O—2′ (“ENA”), 4′—CH(CH₃)—O—2′ (referred to as “constrained ethyl” or “cEt” when in the S configuration), 4′—CH₂—O—CH₂-2′, 4′—CH₂—N(R)-2′, 4′—CH(CH₂OCH₃)—O—2′ (“constrained MOE” or “cMOE”) and analogs thereof (see, e.g., Seth et al., U.S. Pat. No. 7,399,845, Bhat et al., U.S. Pat. No. 7,569,686, Swayze et al., U.S. Pat. No. 7,741,457, and Swayze et al., U.S. Pat. No. 8,022,193), 4′-C(CH₃)(CH)—O—2′ and analogs thereof (see, e.g., Seth et al., U.S. Pat. No. 8,278,283), 4′—CH₂—N(OCH₃)-2′ and analogs thereof (see, e.g., Prakash et al., U.S. Pat. No. 8,278,425), 4′—CH₂—O—N(CH₃)-2′ (see, e.g., Allerson et al., U.S. Pat. No. 7,696,345 and Allerson et al., U.S. Pat. No. 8,124,745), 4′—CH₂—C(H)(CH₃)-2′ (see, e.g., Zhou, et al., J. Org. Chem.,2009, 74, 118-134), 4′—CH₂—C(═CH₂)-2′ and analogs thereof (see e.g., Seth et al., U.S. Pat. No. 8,278,426), 4′-C(RₐR_{b})-N(R)—O—2′, 4′-C(RaR)—O—N(R)-2′, 4′—CH₂—O—N(R)-2′, and 4′—CH₂—N(R)—O—2′, wherein each R, Rₐ, and R_{b} is, independently, H, a protecting group, or Cᵢ-C₁₂ alkyl (see, e.g. Imanishi et al., U.S. Pat. No. 7,427,672).

In certain embodiments, such 4′ to 2′ bridges independently comprise from 1 to 4 linked groups independently selected from: —[C(Rₐ)(R_{b})]ₙ—, —[C(Rₐ)(R)]ₙ—O—, —C(Rₐ)═C(R_{b})—, —C(Rₐ)═N—, —C(═NRₐ)—, —C(═O)—C(═S)—, —O—, —Si(Rₐ)2—, —S(═O)—, and —N(Rₐ)—;
wherein:
x is 0,1, or 2;
n is 1, 2, 3, or 4;
each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl(C(═O)—H), substituted acyl, CN, sulfonyl(S(═O)₂-J₁), or sulfoxyl(S(═O)—J₁); and each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl(C(═O)—H), substituted acyl, a heterocycle radical, a substituted heterocycle radical, C₁-C₁₂ aminoalkyl, substituted C₁-C₁₂ aminoalkyl, or a protecting group.

Additional bicyclic sugar moieties are known in the art, see, for example: Freier et al., Nucleic Acids Research, 1997, 25(22), 4429-4443, Albaek et al., J. Org. Chem., 2006, 71, 7731-7740, Singh et al., Chem. Commun., 1998, 4, 455-456; Koshkin et al., Tetrahedron, 1998, 54, 3607-3630; Wahlestedt et al., Proc. Natl. Acad. Sci. U.S.A, 2000, 97, 5633-5638; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 2007, 129, 8362-8379; Elayadi et al., Curr. Opinion Invens. Drugs, 2001, 2, 558-561; Braasch et al., Chem. Biol., 2001, 8, 1-7; Orum et al., Curr. Opinion Mol. Ther., 2001, 3, 239-243; Wengel et al., U.S. Pat. No. 7,053,207, Imanishi et al., U.S. Pat. No. 6,268,490, Imanishi et al. U.S. Pat. No. 6,770,748, Imanishi et al., U.S. RE44,779; Wengel et al., U.S. Pat. No. 6,794,499, Wengel et al., U.S. Pat. No. 6,670,461; Wengel et al., U.S. Pat. No. 7,034,133, Wengel et al., U.S. Pat. No. 8,080,644; Wengel et al., U.S. Pat. No. 8,034,909; Wengel et al., U.S. Pat. No. 8,153,365; Wengel et al., U.S. Pat. No. 7,572,582; and Ramasamy et al., U.S. Pat. No. 6,525,191, Torsten et al., WO 2004/106356, Wengel et al., WO 1999/014226; Seth et al., WO 2007/134181; Seth et al., U.S. Pat. No. 7,547,684; Seth et al., U.S. Pat. No. 7,666,854; Seth et al., U.S. Pat. No. 8,088,746; Seth et al., U.S. Pat. No. 7,750,131; Seth et al., U.S. Pat. No. 8,030,467; Seth et al., U.S. Pat. No. 8,268,980; Seth et al., U.S. Pat. No. 8,546,556; Seth et al., U.S. Pat. No. 8,530,640; Migawa et al., U.S. Pat. No. 9,012,421; Seth et al., U.S. Pat. No. 8,501,805; Allerson et al., US2008/0039618; and Migawa et al., US2015/0191727.

In certain embodiments, bicyclic sugar moieties and nucleosides incorporating such bicyclic sugar moieties are further defined by isomeric configuration. For example, an LNA nucleoside (described herein) may be in the α-L configuration or in the β-D configuration.

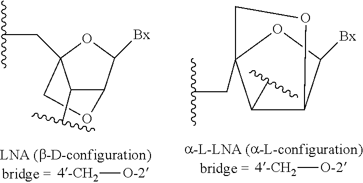

α-L-methyleneoxy (4′—CH₂—O—2′) or α-L-LNA bicyclic nucleosides have been incorporated into oligonucleotides that showed antisense activity (Frieden et al., Nucleic Acids Research, 2003, 21, 6365-6372). Herein, general descriptions of bicyclic nucleosides include both isomeric configurations. When the positions of specific bicyclic nucleosides (e.g., LNA or cEt) are identified in exemplified embodiments herein, they are in the β-D configuration, unless otherwise specified.

In certain embodiments, modified sugar moieties comprise one or more non-bridging sugar substituent and one or more bridging sugar substituent (e.g., 5′-substituted and 4′-2′ bridged sugars).

In certain embodiments, modified sugar moieties are sugar surrogates. In certain such embodiments, the oxygen atom of the sugar moiety is replaced, e.g., with a sulfur, carbon or nitrogen atom. In certain such embodiments, such modified sugar moieties also comprise bridging and/or non-bridging substituents as described herein. For example, certain sugar surrogates comprise a 4′-sulfur atom and a substitution at the 2′-position (see, e.g., Bhat et al., U.S. Pat. No. 7,875,733 and Bhat et al., U.S. Pat. No. 7,939,677) and/or the 5′ position.

In certain embodiments, sugar surrogates comprise rings having other than 5 atoms. For example, in certain embodiments, a sugar surrogate comprises a six-membered tetrahydropyran (“THP”). Such tetrahydropyrans may be further modified or substituted. Nucleosides comprising such modified tetrahydropyrans include, but are not limited to, hexitol nucleic acid (“HNA”), altritol nucleic acid (“ANA”), mannitol nucleic acid (“MNA”) (see e.g., Leumann, CJ. Bioorg. & Med. Chem. 2002, 10, 841-854), fluoro HNA:

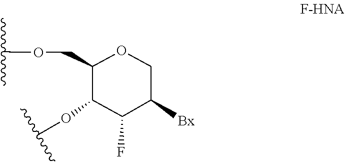

(“F-HNA,” see e.g., Swayze et al., U.S. Pat. No. 8,088,904; Swayze et al., U.S. Pat. No. 8,440,803; and Swayze et al., U.S. Pat. No. 9,005,906) F-HNA can also be referred to as a F-THP or 3′-fluoro tetrahydropyran, and nucleosides comprising additional modified THP compounds having the formula:

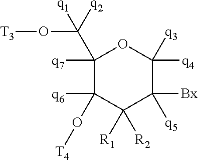

wherein. independently. for each of said modified THP nucleoside:
Bx is a nucleobase moiety;
T₃ and T₄ are each, independently, an internucleoside linking group linking the modified THP nucleoside to the remainder of an oligonucleotide or one of T₃ and T₄ is an internucleoside linking group linking the modified THP nucleoside to the remainder of an oligonucleotide and the other of T₃ and T₄ is H, a hydroxyl protecting group, a linked conjugate group, or a 5′ or 3′-terminal group; q₁, q₂, q₃, q₄, q₅, q₆ and q₇ are each, independently, H, C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl; and each of R₁ and R2 is independently selected from among: hydrogen, halogen, substituted or unsubstituted alkoxy, NJ₁J₂, SJ₁, N₃, OC(═X)J₁, OC(═X)NJ₁J₂, NJ₃C(═X)NJ₁J₂, and CN, wherein X is O, S or NJ₁, and each J₁, J₂, and J₃ is, independently, H or C₁-C₆ alkyl.

In certain embodiments, modified THP nucleosides are provided wherein q₁, q₂, q₃, q₄, q₅, q₆ and q are each H. In certain embodiments, at least one of q₁, q₂, q₃, q₄, q₅, q₆ and q₇ is other than H. In certain embodiments, at least one of q₁, q₂, q₃, q₄, q₅, q₆ and q is methyl. In certain embodiments, modified THP nucleosides are provided wherein one of R₁ and R₂ is F. In certain embodiments, R₁ is F and R₂ is H, in certain embodiments, R₁ is methoxy and R₂ is H, and in certain embodiments, R₁ is methoxyethoxy and R₂ is H.

In certain embodiments, sugar surrogates comprise rings having more than 5 atoms and more than one heteroatom. For example, nucleosides comprising morpholino sugar moieties and their use in oligonucleotides have been reported (see, e.g., Braasch et al., Biochemistry, 2002, 41, 4503-4510 and Summerton et al., U.S. Pat. No. 5,698,685; Summerton et al., U.S. Pat. No. 5,166,315; Summerton et al., U.S. Pat. No. 5,185,444; and Summerton et al., U.S. Pat. No. 5,034,506). As used here, the term “morpholino” means a sugar surrogate having the following structure:

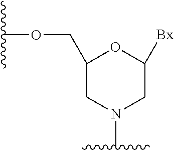

In certain embodiments, morpholinos may be modified, for example, by adding or altering various substituent groups from the above morpholino structure. Such sugar surrogates are referred to herein as “modified morpholinos.”

In certain embodiments, sugar surrogates comprise acyclic moieties. Examples of nucleosides and oligonucleotides comprising such acyclic sugar surrogates include, but are not limited to: peptide nucleic acid (“PNA”), acyclic butyl nucleic acid (see, e.g., Kumar et al., Org. Biomol. Chem., 2013, 11, 5853-5865), and nucleosides and oligonucleotides described in Manoharan et al., US2013/130378.

Many other bicyclic and tricyclic sugar and sugar surrogate ring systems are known in the art that can be used in modified nucleosides.

### 2. Modified Nucleobases

Nucleobase (or base) modifications or substitutions are structurally distinguishable from, yet functionally interchangeable with, naturally occurring or synthetic unmodified nucleobases. Both natural and modified nucleobases are capable of participating in hydrogen bonding. Such nucleobase modifications can impart nuclease stability, binding affinity or some other beneficial biological property to antisense compounds.

In certain embodiments, compounds described herein comprise modified oligonucleotides. In certain embodiments, modified oligonucleotides comprise one or more nucleoside comprising an unmodified nucleobase. In certain embodiments, modified oligonucleotides comprise one or more nucleoside comprising a modified nucleobase. In certain embodiments, modified oligonucleotides comprise one or more nucleosides that does not comprise a nucleobase, referred to as an abasic nucleoside.

In certain embodiments, modified nucleobases are selected from: 5-substituted pyrimidines, 6-azapyrimidines, alkyl or alkynyl substituted pyrimidines, alkyl substituted purines, and N-2, N-6 and 0-6 substituted purines. In certain embodiments, modified nucleobases are selected from: 2-aminopropyladenine, 5-hydroxymethyl cytosine, 5-methylcytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl(C═C-CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly, 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one, and 9-(2-aminoethoxy)-1,3-diazaphenoxazine-2-one (G-clamp). Modified nucleobases may also include those in which the purine or pyrimidine base is replaced with other heterocycles, for example, 7-deaza-adenine, 7-deazaguanosine, 2-aminopyridine and 2-pyridone.

Further nucleobases include those disclosed in Merigan et al., U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

Publications that teach the preparation of certain of the above noted modified nucleobases, as well as other modified nucleobases include without limitation, Manoharan et al., US2003/0158403, Manoharan et al., US2003/0175906; Dinh et al., U.S. Pat. No. 4,845,205; Spielvogel et al., U.S. Pat. No. 5,130,302; Rogers et al., U.S. Pat. No. 5,134,066; Bischofberger et al., U.S. Pat. No. 5,175,273; Urdea et al., U.S. Pat. No. 5,367,066; Benner et al., U.S. Pat. No. 5,432,272; Matteucci et al., U.S. Pat. No. 5,434,257; Gmeiner et al., U.S. Pat. No. 5,457,187; Cook et al., U.S. Pat. No. 5,459,255; Froehler et al., U.S. Pat. No. 5,484,908; Matteucci et al., U.S. Pat. No. 5,502,177; Hawkins et al., U.S. Pat. No. 5,525,711; Haralambidis et al., U.S. Pat. No. 5,552,540; Cook et al., U.S. Pat. No. 5,587,469; Froehler et al., U.S. Pat. No. 5,594,121; Switzer et al., U.S. Pat. No. 5,596,091; Cook et al., U.S. Pat. No. 5,614,617; Froehler et al., U.S. Pat. No. 5,645,985; Cook et al., U.S. Pat. No. 5,681,941; Cook et al., U.S. Pat. No. 5,811,534; Cook et al., U.S. Pat. No. 5,750,692; Cook et al., U.S. Pat. No. 5,948,903; Cook et al., U.S. Pat. No. 5,587,470; Cook et al., U.S. Pat. No. 5,457,191; Matteucci et al., U.S. Pat. No. 5,763,588; Froehler et al., U.S. Pat. No. 5,830,653; Cook et al., U.S. Pat. No. 5,808,027; Cook et al., U.S. Pat. No. 6,166,199; and Matteucci et al., U.S. Pat. No. 6,005,096.

In certain embodiments, compounds targeted to a PNPLA3 nucleic acid comprise one or more modified nucleobases. In certain embodiments, the modified nucleobase is 5-methylcytosine. In certain embodiments, each cytosine is a 5-methylcytosine.

### 3. Modified Internucleoside Linkages

The naturally occurring internucleoside linkage of RNA and DNA is a 3′ to 5′ phosphodiester linkage. In certain embodiments, compounds described herein having one or more modified, i.e. non-naturally occurring, internucleoside linkages are often selected over compounds having naturally occurring internucleoside linkages because of desirable properties such as, for example, enhanced cellular uptake, enhanced affinity for target nucleic acids, and increased stability in the presence of nucleases.

Representative internucleoside linkages having a chiral center include but are not limited to alkylphosphonates and phosphorothioates. Modified oligonucleotides comprising internucleoside linkages having a chiral center can be prepared as populations of modified oligonucleotides comprising stereorandom internucleoside linkages, or as populations of modified oligonucleotides comprising phosphorothioate linkages in particular stereochemical configurations. In certain embodiments, populations of modified oligonucleotides comprise phosphorothioate internucleoside linkages wherein all of the phosphorothioate internucleoside linkages are stereorandom. Such modified oligonucleotides can be generated using synthetic methods that result in random selection of the stereochemical configuration of each phosphorothioate linkage. Nonetheless, as is well understood by those of skill in the art, each individual phosphorothioate of each individual oligonucleotide molecule has a defined stereoconfiguration. In certain embodiments, populations of modified oligonucleotides are enriched for modified oligonucleotides comprising one or more particular phosphorothioate internucleoside linkages in a particular, independently selected stereochemical configuration. In certain embodiments, the particular configuration of the particular phosphorothioate linkage is present in at least 65% of the molecules in the population. In certain embodiments, the particular configuration of the particular phosphorothioate linkage is present in at least 70% of the molecules in the population. In certain embodiments, the particular configuration of the particular phosphorothioate linkage is present in at least 80% of the molecules in the population. In certain embodiments, the particular configuration of the particular phosphorothioate linkage is present in at least 90% of the molecules in the population. In certain embodiments, the particular configuration of the particular phosphorothioate linkage is present in at least 99% of the molecules in the population. Such chirally enriched populations of modified oligonucleotides can be generated using synthetic methods known in the art, e.g., methods described in Oka et al., JACS 125, 8307 (2003), Wan et al. Nuc. Acid. Res. 42, 13456 (2014), and WO 2017/015555. In certain embodiments, a population of modified oligonucleotides is enriched for modified oligonucleotides having at least one indicated phosphorothioate in the (Sp) configuration. In certain embodiments, a population of modified oligonucleotides is enriched for modified oligonucleotides having at least one phosphorothioate in the (Rp) configuration. In certain embodiments, modified oligonucleotides comprising (Rp) and/or (Sp) phosphorothioates comprise one or more of the following formulas, respectively, wherein “B” indicates a nucleobase:

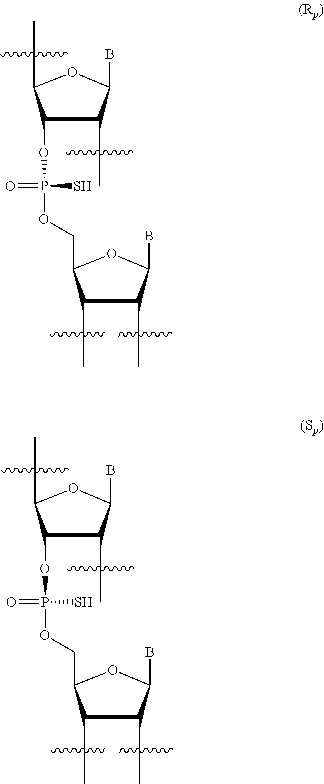

Unless otherwise indicated, chiral internucleoside linkages of modified oligonucleotides described herein can be stereorandom or in a particular stereochemical configuration.

In certain embodiments, compounds targeted to a PNPLA3 nucleic acid comprise one or more modified internucleoside linkages. In certain embodiments, the modified internucleoside linkages are phosphorothioate linkages. In certain embodiments, each internucleoside linkage of an antisense compound is a phosphorothioate internucleoside linkage.

In certain embodiments, compounds described herein comprise oligonucleotides. Oligonucleotides having modified internucleoside linkages include internucleoside linkages that retain a phosphorus atom as well as internucleoside linkages that do not have a phosphorus atom. Representative phosphorus containing internucleoside linkages include, but are not limited to, phosphodiesters, phosphotriesters, methylphosphonates, phosphoramidate, and phosphorothioates. Methods of preparation of phosphorous-containing and non-phosphorous-containing linkages are well known.

In certain embodiments, nucleosides of modified oligonucleotides may be linked together using any internucleoside linkage. The two main classes of internucleoside linking groups are defined by the presence or absence of a phosphorus atom. Representative phosphorus-containing internucleoside linkages include, but are not limited to, phosphates, which contain a phosphodiester bond (“P═O”) (also referred to as unmodified or naturally occurring linkages), phosphotriesters, methylphosphonates, phosphoramidates, and phosphorothioates (“P═S”), and phosphorodithioates (“HS—P═S”). Representative non-phosphorus containing internucleoside linking groups include, but are not limited to, methylenemethylimino (—CH₂—N(CH₃)—O—CH₂), thiodiester, thionocarbamate (—O—C(═O)(NH)—S—); siloxane (—O—SiH₂—O—); and N,N′-dimethylhydrazine (—CH₂—N(CH₃)—N(CH₃)—). Modified internucleoside linkages, compared to naturally occurring phosphate linkages, can be used to alter, typically increase, nuclease resistance of the oligonucleotide. In certain embodiments, internucleoside linkages having a chiral atom can be prepared as a racemic mixture, or as separate enantiomers. Representative chiral internucleoside linkages include, but are not limited to, alkylphosphonates and phosphorothioates. Methods of preparation of phosphorous-containing and non-phosphorous-containing internucleoside linkages are well known to those skilled in the art.

Neutral internucleoside linkages include, without limitation, phosphotriesters, methylphosphonates, MMI (3′—CH₂—N(CH₃)—O—5′), amide-3 (3′—CH₂—C(═O)—N(H)—5′), amide-4 (3′—CH₂—N(H)—C(═O)—5′), formacetal (3′—O—CH₂—O—5′), methoxypropyl, and thioformacetal (3′-S—CH₂—O—5′). Further neutral internucleoside linkages include nonionic linkages comprising siloxane (dialkylsiloxane), carboxylate ester, carboxamide, sulfide, sulfonate ester and amides (See, for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

In certain embodiments, oligonucleotides comprise modified internucleoside linkages arranged along the oligonucleotide or region thereof in a defined pattern or modified internucleoside linkage motif. In certain embodiments, internucleoside linkages are arranged in a gapped motif In such embodiments, the internucleoside linkages in each of two wing regions are different from the internucleoside linkages in the gap region. In certain embodiments, the internucleoside linkages in the wings are phosphodiester and the internucleoside linkages in the gap are phosphorothioate. The nucleoside motif is independently selected, so such oligonucleotides having a gapped internucleoside linkage motif may or may not have a gapped nucleoside motif and, if it does have a gapped nucleoside motif, the wing and gap lengths may or may not be the same.

In certain embodiments, oligonucleotides comprise a region having an alternating internucleoside linkage motif. In certain embodiments, oligonucleotides comprise a region of uniformly modified internucleoside linkages. In certain such embodiments, the oligonucleotide comprises a region that is uniformly linked by phosphorothioate internucleoside linkages. In certain embodiments, the oligonucleotide is uniformly linked by phosphorothioate. In certain embodiments, each internucleoside linkage of the oligonucleotide is selected from phosphodiester and phosphorothioate. In certain embodiments, each internucleoside linkage of the oligonucleotide is selected from phosphodiester and phosphorothioate and at least one internucleoside linkage is phosphorothioate.

In certain embodiments, the oligonucleotide comprises at least 6 phosphorothioate internucleoside linkages. In certain embodiments, the oligonucleotide comprises at least 8 phosphorothioate internucleoside linkages. In certain embodiments, the oligonucleotide comprises at least 10 phosphorothioate internucleoside linkages. In certain embodiments, the oligonucleotide comprises at least one block of at least 6 consecutive phosphorothioate internucleoside linkages. In certain embodiments, the oligonucleotide comprises at least one block of at least 8 consecutive phosphorothioate internucleoside linkages. In certain embodiments, the oligonucleotide comprises at least one block of at least 10 consecutive phosphorothioate internucleoside linkages. In certain embodiments, the oligonucleotide comprises at least block of at least one 12 consecutive phosphorothioate internucleoside linkages. In certain such embodiments, at least one such block is located at the 3′ end of the oligonucleotide. In certain such embodiments, at least one such block is located within 3 nucleosides of the 3′ end of the oligonucleotide.

In certain embodiments, oligonucleotides comprise one or more methylphosphonate linkages. In certain embodiments, oligonucleotides having a gapmer nucleoside motif comprise a linkage motif comprising all phosphorothioate linkages except for one or two methylphosphonate linkages. In certain embodiments, one methylphosphonate linkage is in the central gap of an oligonucleotide having a gapmer nucleoside motif.

In certain embodiments, it is desirable to arrange the number of phosphorothioate internucleoside linkages and phosphodiester internucleoside linkages to maintain nuclease resistance. In certain embodiments, it is desirable to arrange the number and position of phosphorothioate internucleoside linkages and the number and position of phosphodiester internucleoside linkages to maintain nuclease resistance. In certain embodiments, the number of phosphorothioate internucleoside linkages may be decreased and the number of phosphodiester internucleoside linkages may be increased. In certain embodiments, the number of phosphorothioate internucleoside linkages may be decreased and the number of phosphodiester internucleoside linkages may be increased while still maintaining nuclease resistance. In certain embodiments, it is desirable to decrease the number of phosphorothioate internucleoside linkages while retaining nuclease resistance. In certain embodiments, it is desirable to increase the number of phosphodiester internucleoside linkages while retaining nuclease resistance.

### 4. Certain Motifs

In certain embodiments, compounds described herein comprise oligonucleotides. Oligonucleotides can have a motif, e.g. a pattern of unmodified and/or modified sugar moieties, nucleobases, and/or internucleoside linkages. In certain embodiments, modified oligonucleotides comprise one or more modified nucleosides comprising a modified sugar. In certain embodiments, modified oligonucleotides comprise one or more modified nucleosides comprising a modified nucleobase. In certain embodiments, modified oligonucleotides comprise one or more modified internucleoside linkage. In such embodiments, the modified, unmodified, and differently modified sugar moieties, nucleobases, and/or internucleoside linkages of a modified oligonucleotide define a pattern or motif In certain embodiments, the patterns of sugar moieties, nucleobases, and internucleoside linkages are each independent of one another. Thus, a modified oligonucleotide may be described by its sugar motif, nucleobase motif and/or internucleoside linkage motif (as used herein, nucleobase motif describes the modifications to the nucleobases independent of the sequence of nucleobases).

a. Certain Sugar Motifs

In certain embodiments, compounds described herein comprise oligonucleotides. In certain embodiments, oligonucleotides comprise one or more type of modified sugar and/or unmodified sugar moiety arranged along the oligonucleotide or region thereof in a defined pattern or sugar motif. In certain instances, such sugar motifs include, but are not limited to, any of the sugar modifications discussed herein.

In certain embodiments, modified oligonucleotides comprise or consist of a region having a gapmer motif, which comprises two external regions or “wings” and a central or internal region or “gap.” The three regions of a gapmer motif (the 5′-wing, the gap, and the 3′-wing) form a contiguous sequence of nucleosides, wherein at least some of the sugar moieties of the nucleosides of each of the wings differ from at least some of the sugar moieties of the nucleosides of the gap. Specifically, at least the sugar moieties of the nucleosides of each wing that are closest to the gap (the 3′-most nucleoside of the 5′-wing and the 5′-most nucleoside of the 3′-wing) differ from the sugar moiety of the neighboring gap nucleosides, thus defining the boundary between the wings and the gap (i.e., the wing/gap junction). In certain embodiments, the sugar moieties within the gap are the same as one another. In certain embodiments, the gap includes one or more nucleosides having a sugar moiety that differs from the sugar moiety of one or more other nucleosides of the gap. In certain embodiments, the sugar motifs of the two wings are the same as one another (symmetric gapmer). In certain embodiments, the sugar motif of the 5′-wing differs from the sugar motif of the 3′-wing (asymmetric gapmer).

In certain embodiments, the wings of a gapmer comprise 1-5 nucleosides. In certain embodiments, the wings of a gapmer comprise 2-5 nucleosides. In certain embodiments, the wings of a gapmer comprise 3-5 nucleosides. In certain embodiments, the nucleosides of a gapmer are all modified nucleosides.

In certain embodiments, the gap of a gapmer comprises 7-12 nucleosides. In certain embodiments, the gap of a gapmer comprises 7-10 nucleosides. In certain embodiments, the gap of a gapmer comprises 8-10 nucleosides. In certain embodiments, the gap of a gapmer comprises 10 nucleosides. In certain embodiments, each nucleoside of the gap of a gapmer is an unmodified 2′-deoxy nucleoside.

In certain embodiments, the gapmer is a deoxy gapmer. In such embodiments, the nucleosides on the gap side of each wing/gap junction are unmodified 2′-deoxy nucleosides and the nucleosides on the wing sides of each wing/gap junction are modified nucleosides. In certain such embodiments, each nucleoside of the gap is an unmodified 2′-deoxy nucleoside. In certain such embodiments, each nucleoside of each wing is a modified nucleoside.

In certain embodiments, a modified oligonucleotide has a fully modified sugar motif wherein each nucleoside of the modified oligonucleotide comprises a modified sugar moiety. In certain embodiments, modified oligonucleotides comprise or consist of a region having a fully modified sugar motif wherein each nucleoside of the region comprises a modified sugar moiety. In certain embodiments, modified oligonucleotides comprise or consist of a region having a fully modified sugar motif, wherein each nucleoside within the fully modified region comprises the same modified sugar moiety, referred to herein as a uniformly modified sugar motif. In certain embodiments, a fully modified oligonucleotide is a uniformly modified oligonucleotide. In certain embodiments, each nucleoside of a uniformly modified comprises the same 2′-modification.

b. Certain Nucleobase Motifs

In certain embodiments, compounds described herein comprise oligonucleotides. In certain embodiments, oligonucleotides comprise modified and/or unmodified nucleobases arranged along the oligonucleotide or region thereof in a defined pattern or motif. In certain embodiments, each nucleobase is modified. In certain embodiments, none of the nucleobases are modified. In certain embodiments, each purine or each pyrimidine is modified. In certain embodiments, each adenine is modified. In certain embodiments, each guanine is modified. In certain embodiments, each thymine is modified. In certain embodiments, each uracil is modified. In certain embodiments, each cytosine is modified. In certain embodiments, some or all of the cytosine nucleobases in a modified oligonucleotide are 5-methylcytosines.

In certain embodiments, modified oligonucleotides comprise a block of modified nucleobases. In certain such embodiments, the block is at the 3′-end of the oligonucleotide. In certain embodiments, the block is within 3 nucleosides of the 3′-end of the oligonucleotide. In certain embodiments, the block is at the 5′-end of the oligonucleotide. In certain embodiments, the block is within 3 nucleosides of the 5′-end of the oligonucleotide.

In certain embodiments, oligonucleotides having a gapmer motif comprise a nucleoside comprising a modified nucleobase. In certain such embodiments, one nucleoside comprising a modified nucleobase is in the central gap of an oligonucleotide having a gapmer motif. In certain such embodiments, the sugar moiety of said nucleoside is a 2′-deoxyribosyl moiety. In certain embodiments, the modified nucleobase is selected from: a 2-thiopyrimidine and a 5-propynepyrimidine.

c. Certain Internucleoside Linkage Motifs

In certain embodiments, compounds described herein comprise oligonucleotides. In certain embodiments, oligonucleotides comprise modified and/or unmodified internucleoside linkages arranged along the oligonucleotide or region thereof in a defined pattern or motif. In certain embodiments, essentially each internucleoside linking group is a phosphate internucleoside linkage (P═O). In certain embodiments, each internucleoside linking group of a modified oligonucleotide is a phosphorothioate (P═S). In certain embodiments, each internucleoside linking group of a modified oligonucleotide is independently selected from a phosphorothioate and phosphate internucleoside linkage. In certain embodiments, the sugar motif of a modified oligonucleotide is a gapmer and the internucleoside linkages within the gap are all modified. In certain such embodiments, some or all of the internucleoside linkages in the wings are unmodified phosphate linkages. In certain embodiments, the terminal internucleoside linkages are modified. In certain embodiments, the sugar motif of a modified oligonucleotide is a gapmer, and the internucleoside linkage motif comprises at least one phosphodiester internucleoside linkage in at least one wing, wherein the at least one phosphodiester linkage is not a terminal internucleoside linkage, and the remaining internucleoside linkages are phosphorothioate internucleoside linkages. In certain such embodiments, all of the phosphorothioate linkages are stereorandom. In certain embodiments, all of the phosphorothioate linkages in the wings are (Sp) phosphorothioates, and the gap comprises at least one Sp, Sp, Rp motif. In certain embodiments, populations of modified oligonucleotides are enriched for modified oligonucleotides comprising such internucleoside linkage motifs.

### 5. Certain Modified Oligonucleotides

In certain embodiments, compounds described herein comprise modified oligonucleotides. In certain embodiments, the above modifications (sugar, nucleobase, internucleoside linkage) are incorporated into a modified oligonucleotide. In certain embodiments, modified oligonucleotides are characterized by their modification, motifs, and overall lengths. In certain embodiments, such parameters are each independent of one another. Thus, unless otherwise indicated, each internucleoside linkage of an oligonucleotide having a gapmer sugar motif may be modified or unmodified and may or may not follow the gapmer modification pattern of the sugar modifications. For example, the internucleoside linkages within the wing regions of a sugar gapmer may be the same or different from one another and may be the same or different from the internucleoside linkages of the gap region of the sugar motif. Likewise, such gapmer oligonucleotides may comprise one or more modified nucleobases independent of the gapmer pattern of the sugar modifications. Furthermore, in certain instances, an oligonucleotide is described by an overall length or range and by lengths or length ranges of two or more regions (e.g., a region of nucleosides having specified sugar modifications). In such circumstances, it may be possible to select numbers for each range that result in an oligonucleotide having an overall length falling outside the specified range. In such circumstances, both elements must be satisfied. For example, in certain embodiments, a modified oligonucleotide consists of 15-20 linked nucleosides and has a sugar motif consisting of three regions, A, B, and C, wherein region A consists of 2-6 linked nucleosides having a specified sugar motif, region B consists of 6-10 linked nucleosides having a specified sugar motif, and region C consists of 2-6 linked nucleosides having a specified sugar motif. Such embodiments do not include modified oligonucleotides where A and C each consist of 6 linked nucleosides and B consists of 10 linked nucleosides (even though those numbers of nucleosides are permitted within the requirements for A, B, and C) because the overall length of such oligonucleotide will be 22, which exceeds the upper limit of the overall length of the modified oligonucleotide (20). Herein, if a description of an oligonucleotide is silent with respect to one or more parameters, such parameter is not limited. Thus, a modified oligonucleotide described only as having a gapmer sugar motif without further description may have any length, internucleoside linkage motif, and nucleobase motif. Unless otherwise indicated, all modifications are independent of nucleobase sequence.

### Certain Conjugated Compounds

In certain embodiments, the compounds described herein comprise or consist of an oligonucleotide (modified or unmodified) and, optionally, one or more conjugate groups and/or terminal groups. Conjugate groups consist of one or more conjugate moiety and a conjugate linker which links the conjugate moiety to the oligonucleotide. Conjugate groups may be attached to either or both ends of an oligonucleotide and/or at any internal position. In certain embodiments, conjugate groups are attached to the 2′-position of a nucleoside of a modified oligonucleotide. In certain embodiments, conjugate groups that are attached to either or both ends of an oligonucleotide are terminal groups. In certain such embodiments, conjugate groups or terminal groups are attached at the 3′ and/or 5′-end of oligonucleotides. In certain such embodiments, conjugate groups (or terminal groups) are attached at the 3′-end of oligonucleotides. In certain embodiments, conjugate groups are attached near the 3′-end of oligonucleotides. In certain embodiments, conjugate groups (or terminal groups) are attached at the 5′-end of oligonucleotides. In certain embodiments, conjugate groups are attached near the 5′-end of oligonucleotides.

In certain embodiments, the oligonucleotide is modified. In certain embodiments, the oligonucleotide of a compound has a nucleobase sequence that is complementary to a target nucleic acid. In certain embodiments, oligonucleotides are complementary to a messenger RNA (mRNA). In certain embodiments, oligonucleotides are complementary to a pre-mRNA. In certain embodiments, oligonucleotides are complementary to a sense transcript.

Examples of terminal groups include but are not limited to conjugate groups, capping groups, phosphate moieties, protecting groups, modified or unmodified nucleosides, and two or more nucleosides that are independently modified or unmodified.

### Certain Conjugate Groups

In certain embodiments, oligonucleotides are covalently attached to one or more conjugate groups. In certain embodiments, conjugate groups modify one or more properties of the attached oligonucleotide, including, but not limited to, pharmacodynamics, pharmacokinetics, stability, binding, absorption, tissue distribution, cellular distribution, cellular uptake, charge and clearance. In certain embodiments, conjugate groups impart a new property on the attached oligonucleotide, e.g., fluorophores or reporter groups that enable detection of the oligonucleotide.

Certain conjugate groups and conjugate moieties have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., FMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic, a palmityl moiety (Mishra et al., Biochim. Biophys. Acta, 1995, 1264, 229-237), an octadecylamine or hexylamino-carbonyl-oxycholesterol moiety (Crooke et al., J. Pharmacol. Exp. Ther., 1996, i, 923-937), a tocopherol group (Nishina et al., Molecular Therapy Nucleic Acids, 2015, 4, e220; doi: 10.1038/mtna.2014.72 and Nishina et al., Molecular Therapy, 2008, 16, 734-740), or a GalNAc cluster (e.g., WO2014/179620).

### 1. Conjugate Moieties

Conjugate moieties include, without limitation, intercalators, reporter molecules, polyamines, polyamides, peptides, carbohydrates (e.g., GalNAc), vitamin moieties, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, fluorophores, and dyes.

In certain embodiments, a conjugate moiety comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial, or an antibiotic.

2. Conjugate linkers

Conjugate moieties are attached to oligonucleotides through conjugate linkers. In certain embodiments, a conjugate group is a single chemical bond (i.e. conjugate moiety is attached to an oligonucleotide via a conjugate linker through a single bond). In certain embodiments, the conjugate linker comprises a chain structure, such as a hydrocarbyl chain, or an oligomer of repeating units, such as ethylene glycol, nucleosides, or amino acid units.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate groups to parent compounds, such as the oligonucleotides provided herein. In general, a bifunctional linking moiety comprises at least two functional groups. One of the functional groups is selected to bind to a particular site on a compound and the other is selected to bind to a conjugate group. Examples of functional groups used in a bifunctional linking moiety include, but are not limited to, electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include, but are not limited to, pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include, but are not limited to, substituted or unsubstituted C₁-C₁₀ alkyl, substituted or unsubstituted C₂-C₁₀ alkenyl, or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl, and alkynyl.

In certain embodiments, conjugate linkers comprise 1-10 linker-nucleosides. In certain embodiments, such linker-nucleosides are modified nucleosides. In certain embodiments, such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methylcytosine, 4-N-benzoyl-5-methylcytosine, adenine, 6-N-benzoyladenine, guanine and 2-N-isobutyrylguanine. It is typically desirable for linker-nucleosides to be cleaved from the compound after it reaches a target tissue. Accordingly, linker-nucleosides are typically linked to one another and to the remainder of the compound through cleavable bonds. In certain embodiments, such cleavable bonds are phosphodiester bonds.

Herein, linker-nucleosides are not considered to be part of the oligonucleotide. Accordingly, in embodiments in which a compound comprises an oligonucleotide consisting of a specified number or range of linked nucleosides and/or a specified percent complementarity to a reference nucleic acid and the compound also comprises a conjugate group comprising a conjugate linker comprising linker-nucleosides, those linker-nucleosides are not counted toward the length of the oligonucleotide and are not used in determining the percent complementarity of the oligonucleotide for the reference nucleic acid. For example, a compound may comprise (1) a modified oligonucleotide consisting of 8-30 nucleosides and (2) a conjugate group comprising 1-10 linker-nucleosides that are contiguous with the nucleosides of the modified oligonucleotide. The total number of contiguous linked nucleosides in such a compound is more than 30. Alternatively, a compound may comprise a modified oligonucleotide consisting of 8-30 nucleosides and no conjugate group. The total number of contiguous linked nucleosides in such a compound is no more than 30. Unless otherwise indicated, conjugate linkers comprise no more than 10 linker-nucleosides. In certain embodiments, conjugate linkers comprise no more than 5 linker-nucleosides. In certain embodiments, conjugate linkers comprise no more than 3 linker-nucleosides. In certain embodiments, conjugate linkers comprise no more than 2 linker-nucleosides. In certain embodiments, conjugate linkers comprise no more than 1 linker-nucleoside.

In certain embodiments, it is desirable for a conjugate group to be cleaved from the oligonucleotide. For example, in certain circumstances, compounds comprising a particular conjugate moiety are better taken up by a particular cell type, but once the compound has been taken up, it is desirable that the conjugate group be cleaved to release the unconjugated or parent oligonucleotide. Thus, certain conjugates may comprise one or more cleavable moieties, typically within the conjugate linker. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate linkage between an oligonucleotide and a conjugate moiety or conjugate group.

In certain embodiments, a cleavable moiety comprises or consists of one or more linker-nucleosides. In certain such embodiments, one or more linker-nucleosides are linked to one another and/or to the remainder of the compound through cleavable bonds. In certain embodiments, such cleavable bonds are unmodified phosphodiester bonds. In certain embodiments, a cleavable moiety is 2′-deoxy nucleoside that is attached to either the 3′ or 5′-terminal nucleoside of an oligonucleotide by a phosphate internucleoside linkage and covalently attached to the remainder of the conjugate linker or conjugate moiety by a phosphate or phosphorothioate linkage. In certain such embodiments, the cleavable moiety is 2′-deoxyadenosine.

### 3. Certain Cell-Targeting Conjugate Moieties

In certain embodiments, a conjugate group comprises a cell-targeting conjugate moiety. In certain embodiments, a conjugate group has the general formula:

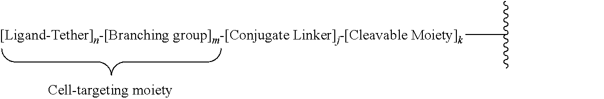

wherein n is from 1 to about 3, m is 0 when n is 1, m is 1 when n is 2 or greater, j is 1 or 0, and k is 1 or 0.

In certain embodiments, n is 1, j is 1 and k is 0. In certain embodiments, n is 1, j is 0 and k is 1. In certain embodiments, n is 1, j is 1 and k is 1. In certain embodiments, n is 2, j is 1 and k is 0. In certain embodiments, n is 2, j is 0 and k is 1. In certain embodiments, n is 2, j is 1 and k is 1. In certain embodiments, n is 3, j is 1 and k is 0. In certain embodiments, n is 3, j is 0 and k is 1. In certain embodiments, n is 3, j is 1 and k is 1.

In certain embodiments, conjugate groups comprise cell-targeting moieties that have at least one tethered ligand. In certain embodiments, cell-targeting moieties comprise two tethered ligands covalently attached to a branching group. In certain embodiments, cell-targeting moieties comprise three tethered ligands covalently attached to a branching group.

In certain embodiments, the cell-targeting moiety comprises a branching group comprising one or more groups selected from alkyl, amino, oxo, amide, disulfide, polyethylene glycol, ether, thioether, and hydroxylamino groups. In certain embodiments, the branching group comprises a branched aliphatic group comprising groups selected from alkyl, amino, oxo, amide, disulfide, polyethylene glycol, ether, thioether, and hydroxylamino groups. In certain such embodiments, the branched aliphatic group comprises groups selected from alkyl, amino, oxo, amide, and ether groups. In certain such embodiments, the branched aliphatic group comprises groups selected from alkyl, amino, and ether groups. In certain such embodiments, the branched aliphatic group comprises groups selected from alkyl and ether groups. In certain embodiments, the branching group comprises a mono or polycyclic ring system.

In certain embodiments, each tether of a cell-targeting moiety comprises one or more groups selected from alkyl, substituted alkyl, ether, thioether, disulfide, amino, oxo, amide, phosphodiester, and polyethylene glycol, in any combination. In certain embodiments, each tether is a linear aliphatic group comprising one or more groups selected from alkyl, ether, thioether, disulfide, amino, oxo, amide, and polyethylene glycol, in any combination. In certain embodiments, each tether is a linear aliphatic group comprising one or more groups selected from alkyl, phosphodiester, ether, amino, oxo, and amide, in any combination. In certain embodiments, each tether is a linear aliphatic group comprising one or more groups selected from alkyl, ether, amino, oxo, and amide, in any combination. In certain embodiments, each tether is a linear aliphatic group comprising one or more groups selected from alkyl, amino, and oxo, in any combination. In certain embodiments, each tether is a linear aliphatic group comprising one or more groups selected from alkyl and oxo, in any combination. In certain embodiments, each tether is a linear aliphatic group comprising one or more groups selected from alkyl and phosphodiester, in any combination. In certain embodiments, each tether comprises at least one phosphorus linking group or neutral linking group. In certain embodiments, each tether comprises a chain from about 6 to about 20 atoms in length. In certain embodiments, each tether comprises a chain from about 10 to about 18 atoms in length. In certain embodiments, each tether comprises about 10 atoms in chain length.

In certain embodiments, each ligand of a cell-targeting moiety has an affinity for at least one type of receptor on a target cell. In certain embodiments, each ligand has an affinity for at least one type of receptor on the surface of a mammalian liver cell. In certain embodiments, each ligand has an affinity for the hepatic asialoglycoprotein receptor (ASGP-R). In certain embodiments, each ligand is a carbohydrate. In certain embodiments, each ligand is, independently selected from galactose, N-acetyl galactoseamine (GalNAc), mannose, glucose, glucoseamine, and fucose. In certain embodiments, each ligand is N-acetyl galactoseamine (GalNAc). In certain embodiments, the cell-targeting moiety comprises 3 GalNAc ligands. In certain embodiments, the cell-targeting moiety comprises 2 GalNAc ligands. In certain embodiments, the cell-targeting moiety comprises 1 GalNAc ligand.

In certain embodiments, each ligand of a cell-targeting moiety is a carbohydrate, carbohydrate derivative, modified carbohydrate, polysaccharide, modified polysaccharide, or polysaccharide derivative. In certain such embodiments, the conjugate group comprises a carbohydrate cluster (see, e.g., Maier et al., “Synthesis of Antisense Oligonucleotides Conjugated to a Multivalent Carbohydrate Cluster for Cellular Targeting,” Bioconjugate Chemistry, 2003, 14, 18-29, or Rensen et al., “Design and Synthesis of Novel N-Acetylgalactosamine-Terminated Glycolipids for Targeting of Lipoproteins to the Hepatic Asiaglycoprotein Receptor,” J. Med. Chem. 2004, 47, 5798-5808, which are incorporated herein by reference in their entirety). In certain such embodiments, each ligand is an amino sugar or a thio sugar. For example, amino sugars may be selected from any number of compounds known in the art, such as sialic acid, α-D-galactosamine, β-muramic acid, 2-deoxy-2-methylamino-L-glucopyranose, 4,6-dideoxy-4-formamido-2,3-di-O-methyl-D-mannopyranose, 2-deoxy-2-sulfoamino-D-glucopyranose and N-sulfo-D-glucosamine, and N-glycoloyl-α-neuraminic acid. For example, thio sugars may be selected from 5-Thio-β-D-glucopyranose, methyl 2,3,4-tri-O-acetyl-1-thio-6-O-trityl-α-D-glucopyranoside, 4-thio-β-D-galactopyranose, and ethyl 3,4,6,7-tetra-O-acetyl-2-deoxy-1,5-dithio-α-D-gluco-heptopyranoside.

In certain embodiments, conjugate groups comprise a cell-targeting moiety having the formula:

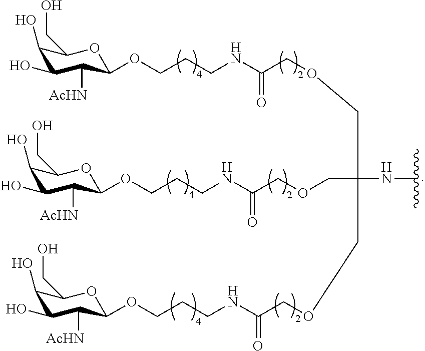

In certain embodiments, conjugate groups comprise a cell-targeting moiety having the formula:

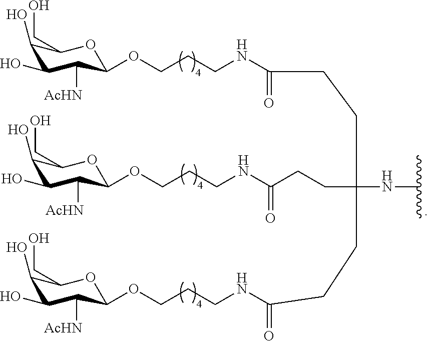

In certain embodiments, conjugate groups comprise a cell-targeting moiety having the formula:

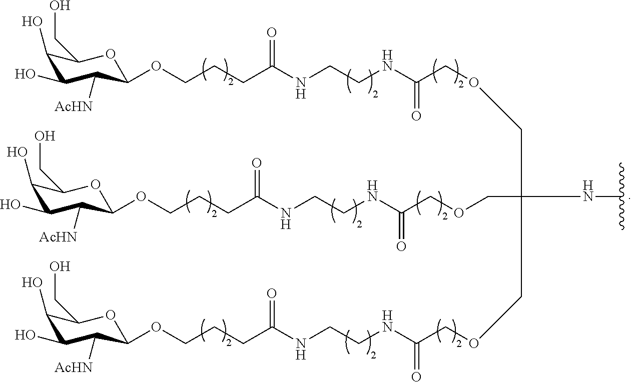

In certain embodiments, compounds described herein comprise a conjugate group described herein as “LICA-1.” LICA-1 is shown below without the optional cleavable moiety at the end of the conjugate linker:

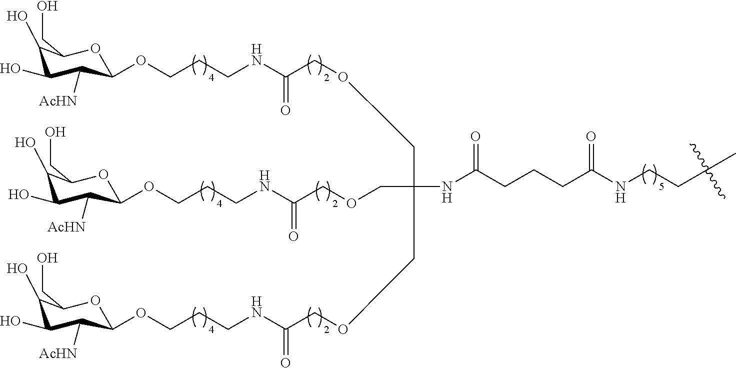

In certain embodiments, compounds described herein comprise LICA-1 and a cleavable moiety within the conjugate linker have the formula:

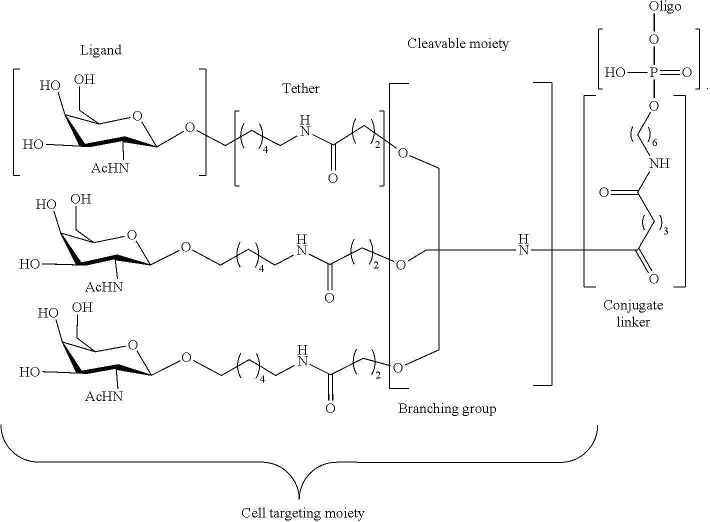

wherein ‘oligo’ is an oligonucleotide.

Representative publications that teach the preparation of certain of the above noted conjugate groups and compounds comprising conjugate groups, tethers, conjugate linkers, branching groups, ligands, cleavable moieties as well as other modifications include, without limitation, U.S. Pat. Nos. 5,994,517, 6,300,319, 6,660,720, 6,906,182, 7,262,177, 7,491,805, 8,106,022, 7,723,509, 9,127,276, US 2006/0148740, US 2011/0123520, WO 2013/033230 and WO 2012/037254, Biessen et al., J. Med. Chem. 1995, 38, 1846-1852, Lee et al., Bioorganic & Medicinal Chemistry 2011,19, 2494-2500, Rensen et al., J. Biol. Chem. 2001, 276, 37577-37584, Rensen et al., J. Med. Chem. 2004, 47, 5798-5808, Sliedregt et al., J. Med. Chem. 1999, 42, 609-618, and Valentijn et al., Tetrahedron, 1997, 53, 759-770, each of which is incorporated by reference herein in its entirety.

In certain embodiments, compounds described herein comprise modified oligonucleotides comprising a gapmer or fully modified motif and a conjugate group comprising at least one, two, or three GalNAc ligands. In certain embodiments, compounds described herein comprise a conjugate group found in any of the following references: Lee, Carbohydr Res, 1978, 67, 509-514; Connolly et al., J Biol Chem, 1982, 257, 939-945; Pavia et al., Int J Pep Protein Res, 1983, 22, 539-548; Lee et al., Biochem, 1984, 23, 4255-4261; Lee et al., Glycoconjugate J, 1987, 4, 317-328; Toyokuni et al., Tetrahedron Lett, 1990, 31, 2673-2676; Biessen et al., J Med Chem, 1995, 38, 1538-1546; Valentijn et al., Tetrahedron, 1997, 53, 759-770; Kim et al., Tetrahedron Lett, 1997, 38, 3487-3490; Lee et al., Bioconjug Chem, 1997, 8, 762-765; Kato et al., Glycobiol, 2001, 11, 821-829; Rensen et al., J Biol Chem, 2001, 276, 37577-37584; Lee et al., Methods Enzymol, 2003, 362, 38-43; Westerlind et al., Glycoconj J, 2004, 21, 227-241; Lee et al., BioorgMed Chem Lett, 2006, 16(19), 5132-5135; Maierhofer et al., BioorgMed Chem, 2007, 15, 7661-7676; Khorev et al., BioorgMed Chem, 2008, 16, 5216-5231; Lee et al., BioorgMed Chem, 2011, 19, 2494-2500; Korilova et al., Analyt Biochem, 2012, 425, 43-46; Pujol et al., Angew Chemie Int Ed Engl, 2012, 51, 7445-7448; Biessen et al., J Med Chem, 1995, 38, 1846-1852; Sliedregt et al., J Med Chem, 1999, 42, 609-618; Rensen et al., J Med Chem, 2004, 47, 5798-5808; Rensen et al., Arterioscler Thromb Vasc Biol, 2006, 26, 169-175; van Rossenberg et al., Gene Ther, 2004, 11, 457-464; Sato et al., JAm Chem Soc, 2004, 126, 14013-14022; Lee et al., J Org Chem, 2012, 77, 7564-7571; Biessen et al., FASEB J, 2000, 14, 1784-1792; Rajur et al., Bioconjug Chem, 1997, 8, 935-940; Duff et al., Methods Enzymol, 2000, 313, 297-321; Maier et al., Bioconjug Chem, 2003, 14, 18-29; Jayaprakash et al., Org Lett, 2010, 12, 5410-5413; Manoharan, Antisense Nucleic Acid Drug Dev, 2002, 12, 103-128; Merwin et al., Bioconjug Chem, 1994, 5, 612-620; Tomiya et al., Bioorg Med Chem, 2013, 21, 5275-5281; International applications WO1998/013381; WO2011/038356; WO1997/046098; WO2008/098788; WO2004/101619; WO2012/037254; WO2011/120053; WO2011/100131; WO2011/163121; WO2012/177947; WO2013/033230; WO2013/075035; WO2012/083185; WO2012/083046; WO2009/082607; WO2009/134487; WO2010/144740; WO2010/148013; WO1997/020563; WO2010/088537; WO2002/043771; WO2010/129709; WO2012/068187; WO2009/126933; WO2004/024757; WO2010/054406; WO2012/089352; WO2012/089602; WO2013/166121; WO2013/165816; U.S. Pat. Nos. 4,751,219; 8,552,163; 6,908,903; 7,262,177; 5,994,517; 6,300,319; 8,106,022; 7,491,805; 7,491,805; 7,582,744; 8,137,695; 6,383,812; 6,525,031; 6,660,720; 7,723,509; 8,541,548; 8,344,125; 8,313,772; 8,349,308; 8,450,467; 8,501,930; 8,158,601; 7,262,177; 6,906,182; 6,620,916; 8,435,491; 8,404,862; 7,851,615; Published U.S. Patent Application Publications US2011/0097264; US2011/0097265; US2013/0004427; US2005/0164235; US2006/0148740; US2008/0281044; US2010/0240730; US2003/0119724; US2006/0183886; US2008/0206869; US2011/0269814; US2009/0286973; US2011/0207799; US2012/0136042; US2012/0165393; US2008/0281041; US2009/0203135; US2012/0035115; US2012/0095075; US2012/0101148; US2012/0128760; US2012/0157509; US2012/0230938; US2013/0109817; US2013/0121954; US2013/0178512; US2013/0236968; US2011/0123520; US2003/0077829; US2008/0108801; and US2009/0203132; each of which is incorporated by reference in its entirety.

### Compositions and Methods for Formulating Pharmaceutical Compositions

Compounds described herein may be admixed with pharmaceutically acceptable active or inert substances for the preparation of pharmaceutical compositions or formulations. Compositions and methods for the formulation of pharmaceutical compositions are dependent upon a number of criteria, including, but not limited to, route of administration, extent of disease, or dose to be administered.

Certain embodiments provide pharmaceutical compositions comprising one or more compounds or a salt thereof. In certain embodiments, the compounds are antisense compounds or oligomeric compounds. In certain embodiments, the compounds comprise or consist of a modified oligonucleotide. In certain such embodiments, the pharmaceutical composition comprises a suitable pharmaceutically acceptable diluent or carrier. In certain embodiments, a pharmaceutical composition comprises a sterile saline solution and one or more compound. In certain embodiments, such pharmaceutical composition consists of a sterile saline solution and one or more compound. In certain embodiments, the sterile saline is pharmaceutical grade saline. In certain embodiments, a pharmaceutical composition comprises one or more compound and sterile water. In certain embodiments, a pharmaceutical composition consists of one compound and sterile water. In certain embodiments, the sterile water is pharmaceutical grade water. In certain embodiments, a pharmaceutical composition comprises one or more compounds and phosphate-buffered saline (PBS). In certain embodiments, a pharmaceutical composition consists of one or more compound and sterile PBS. In certain embodiments, the sterile PBS is pharmaceutical grade PBS. Compositions and methods for the formulation of pharmaceutical compositions are dependent upon a number of criteria, including, but not limited to, route of administration, extent of disease, or dose to be administered.

A compound described herein targeted to PNPLA3 nucleic acid can be utilized in pharmaceutical compositions by combining the compound with a suitable pharmaceutically acceptable diluent or carrier. In certain embodiments, a pharmaceutically acceptable diluent is water, such as sterile water suitable for injection. Accordingly, in one embodiment, employed in the methods described herein is a pharmaceutical composition comprising a compound targeted to PNPLA3 nucleic acid and a pharmaceutically acceptable diluent. In certain embodiments, the pharmaceutically acceptable diluent is water. In certain embodiments, the compound comprises or consists of a modified oligonucleotide provided herein.

Pharmaceutical compositions comprising compounds provided herein encompass any pharmaceutically acceptable salts, esters, or salts of such esters, or any other oligonucleotide which, upon administration to an animal, including a human, is capable of providing (directly or indirectly) the biologically active metabolite or residue thereof. In certain embodiments, the compounds are antisense compounds or oligomeric compounds. In certain embodiments, the compound comprises or consists of a modified oligonucleotide. Accordingly, for example, the disclosure is also drawn to pharmaceutically acceptable salts of compounds, prodrugs, pharmaceutically acceptable salts of such prodrugs, and other bioequivalents. Suitable pharmaceutically acceptable salts include, but are not limited to, sodium and potassium salts.

A prodrug can include the incorporation of additional nucleosides at one or both ends of a compound which are cleaved by endogenous nucleases within the body, to form the active compound.

In certain embodiments, the compounds or compositions further comprise a pharmaceutically acceptable carrier or diluent.

### Certain Selected Compounds

Approximately 2,384 newly designed compounds of various lengths, chemistries, and motifs were tested for their effect on human PNPLA3 mRNA in vitro in several cell types (Example 1). Of 2,384 compounds tested for potency at a single dose in vitro, over 400 selected compounds were tested for dose dependent inhibition in A431 cells (Example 2). Of the over 400 compounds tested by dose response assays, the compounds were further screened for high dose tolerability in a BALB/c mouse model and 87 oligonucleotides were selected for in vivo efficacy in a PNPLA3 transgenic mouse model.

Of the 87 oligonucleotides tested in the transgenic mouse model, 23 oligonucleotides were selected to be further tested for tolerability in preclinical rodel models. In the in vivo rodent tolerability models, body weights and organ weights, liver function markers (such as alanine transaminase, aspartate transaminase and bilirubin), and kidney function markers (such as BUN and creatinine) were measured. In the CD1 mouse model and in the Sprague-Dawley rat model, ION 975591, 975605, 975612, 975613, 975616, 975617, 975735, 975736, 994282, and 994284 were found tolerable (Examples 5 and 6).

These compounds were further tested for efficacy in multi-dose assays in PNPLA3 transgenic mice (Example 7).

IONs 994284, 97605, 975616, 994282, 975613, 975617, 975735, 975736, and 975612 were tested for tolerability in cynomolgus monkeys (Example 8). Treatment with the compounds was well tolerated in the monkeys.

Accordingly, provided herein are compounds with any one or more of the improved properties. In certain embodiments, the compounds as described herein are potent and tolerable.

### EXAMPLES

The Examples below describe the screening process to identify lead compounds targeted to PNPLA3. ION 994284, 97605, 975616, 994282, 975613, 975617, 975735, 975736, and 975612 resulted in high potency and tolerability.

### Non-Limiting Disclosure and Incorporation by Reference

Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH sugar moiety and a thymine base could be described as a DNA having a modified sugar (2′-OH for the natural 2′-H of DNA) or as an RNA having a modified base (thymine (methylated uracil) for natural uracil of RNA).

Accordingly, nucleic acid sequences provided herein, including, but not limited to, those in the sequence listing, are intended to encompass nucleic acids containing any combination of natural or modified RNA and/or DNA, including, but not limited to, such nucleic acids having modified nucleobases. By way of further example and without limitation, an oligonucleotide having the nucleobase sequence “ATCGATCG” encompasses any oligonucleotides having such nucleobase sequence, whether modified or unmodified, including, but not limited to, such compounds comprising RNA bases, such as those having sequence “AUCGAUCG” and those having some DNA bases and some RNA bases such as “AUCGATCG” and compounds having other modified nucleobases, such as “ATmCGAUCG,” wherein ^{m}C indicates a cytosine base comprising a methyl group at the 5-position.

Certain compounds described herein (e.g. modified oligonucleotides) have one or more asymmetric centers and thus give rise to enantiomers, diastereomers, and other stereoisomeric configurations that may be defined, in terms of absolute stereochemistry, as (R) or (S), as a or P, such as for sugar anomers, or as (D) or (L), such as for amino acids, etc. Compounds provided herein that are drawn or described as having certain stereoisomeric configurations include only the indicated compounds. Compounds provided herein that are drawn or described with undefined stereochemistry include all such possible isomers, including their stereorandom and optically pure forms. Likewise, all tautomeric forms of the compounds provided herein are included unless otherwise indicated. Unless otherwise indicated, oligomeric compounds and modified oligonucleotides described herein are intended to include corresponding salt forms.

Compounds described herein include variations in which one or more atoms are replaced with a non-radioactive isotope or radioactive isotope of the indicated element. For example, compounds herein that comprise hydrogen atoms encompass all possible deuterium substitutions for each of the ¹H hydrogen atoms. Isotopic substitutions encompassed by the compounds herein include, but are not limited to: ²H or ³H in place of ¹H, ¹³C or ¹⁴C in place of ¹²C, ¹⁵N in place of ¹⁴N, ¹⁷O or ¹⁸O in place of ¹⁶O, and ³³S, ³⁴S, ³⁵S, or ³⁶S in place of ³²S.

While certain compounds, compositions and methods described herein have been described with specificity in accordance with certain embodiments, the following examples serve only to illustrate the compounds described herein and are not intended to limit the same. Each of the references recited in the present application is incorporated herein by reference in its entirety.

### Example 1: Antisense Inhibition of Human PNPLA3 in A431 Cells

Antisense oligonucleotides were designed targeting a PNPLA3 nucleic acid and were tested for their effects on PNPLA3 mRNA in vitro. The antisense oligonucleotides were tested in a series of experiments that had similar culture conditions. The results for each experiment are presented in separate tables shown below.

The newly designed chimeric antisense oligonucleotides in the Tables below were designed as 3-10-3 cEt gapmers. The gapmers are 16 nucleosides in length, wherein the central gap segment comprises of ten 2′-deoxynucleosides and is flanked by wing segments on the 5′ direction and the 3′ direction comprising three nucleosides. Each nucleoside in the 5′ wing segment and each nucleoside in the 3′ wing segment has a cEt sugar modification. The internucleoside linkages throughout each gapmer are phosphorothioate (P═S) linkages. All cytosine residues throughout each gapmer are 5-methylcytosines.

“Start site” indicates the 5′-most nucleoside to which the gapmer is targeted in the human gene sequence. “Stop site” indicates the 3′-most nucleoside to which the gapmer is targeted human gene sequence. Each gapmer listed in the Tables below is targeted to either the human PNPLA3 mRNA, designated herein as SEQ ID NO: 1 (GENBANK Accession No. NM_025225.2) or the human PNPLA3 genomic sequence, designated herein as SEQ ID NO: 2 (GENBANK Accession No. NC_000022.11 truncated from nucleotides 43921001 to 43954500). ‘n/a’ indicates that the antisense oligonucleotide does not target that particular gene sequence with 100% complementarity.

### Study 1

Cultured A431 cells at a density of 20,000 cells per well were transfected by free uptake with 4,000 nM antisense oligonucleotide. After a treatment period of approximately 24 hours, RNA was isolated from the cells and PNPLA3 mRNA levels were measured by quantitative real-time PCR. Human primer probe set RTS36070 (forward sequence CCTTGGTATGTTCCTGCTTCA, designated herein as SEQ ID NO: 11; reverse sequence GTTGTCACTCACTCCTCCATC, designated herein as SEQ ID NO: 12; probe sequence TGGCCTTATCCCTCCTTCCTTCAGA, designated herein as SEQ ID NO: 13) was used to measure mRNA levels. PNPLA3 mRNA levels were adjusted according to total RNA content, as measured by RIBOGREEN®. Results are presented as percent inhibition of PNPLA3, relative to untreated control cells.

TABLE 1
Inhibition of PNPLA3 mRNA by 3-10-3 cEt gapmers targeting SEQ ID NO: 1 and 2

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | PNPLA3 | SEQ |
| Compound | Start | Stop | Start | Stop | | % | ID |
| Number | Site | Site | Site | Site | Sequence (5′ to 3′) | Inhibition | NO |
| 912709 | 27 | 42 | 2765 | 2780 | GGCATTCCCAGCGCGA | 0 | 17 |
| 912710 | 95 | 110 | 2833 | 2848 | TCCTGATCCGCAGCAG | 15 | 18 |
| 912711 | 103 | 118 | 2841 | 2856 | GGCTCGGGTCCTGATC | 0 | 19 |
| 912712 | 131 | 146 | 2869 | 2884 | GTTAGGATCTGGGTCG | 91 | 20 |
| 912713 | 164 | 179 | 2902 | 2917 | GTACATGGCGGCGGCG | 0 | 21 |
| 912714 | 183 | 198 | 2921 | 2936 | TCCAGCCGCGCTCTGC | 23 | 22 |
| 912715 | 196 | 211 | 2934 | 2949 | GCGAAGGACAAGCTCC | 60 | 23 |
| 912716 | 197 | 212 | 2935 | 2950 | CGCGAAGGACAAGCTC | 0 | 24 |
| 912717 | 272 | 287 | 3010 | 3025 | GCGGAGGAGGTGCGGG | 0 | 25 |
| 912718 | 273 | 288 | 3011 | 3026 | CGCGGAGGAGGTGCGG | 0 | 26 |
| 912719 | 274 | 289 | 3012 | 3027 | TCGCGGAGGAGGTGCG | 19 | 27 |
| 912720 | 290 | 305 | 3028 | 3043 | GAACAACATGCGCGCG | 0 | 28 |
| 912721 | 291 | 306 | 3029 | 3044 | CGAACAACATGCGCGC | 7 | 29 |
| 912722 | 292 | 307 | 3030 | 3045 | CCGAACAACATGCGCG | 21 | 30 |
| 912723 | 293 | 308 | 3031 | 3046 | GCCGAACAACATGCGC | 0 | 31 |
| 912724 | 294 | 309 | 3032 | 3047 | CGCCGAACAACATGCG | 0 | 32 |
| 912725 | 323 | 338 | 3061 | 3076 | GCCGACGCAGTGCAAC | 0 | 33 |
| 912726 | 324 | 339 | 3062 | 3077 | CGCCGACGCAGTGCAA | 0 | 34 |
| 912727 | 340 | 355 | 3078 | 3093 | GGGATACCGGAGAGGA | 43 | 35 |
| 912728 | 370 | 385 | 5944 | 5959 | TCTGAGAGGACCTGCA | 53 | 36 |
| 912729 | 375 | 390 | 5949 | 5964 | CAAGATCTGAGAGGAC | 64 | 37 |
| 912730 | 404 | 419 | 5978 | 5993 | GCCAATGTTCCGACTC | 71 | 38 |
| 912731 | 410 | 425 | 5984 | 5999 | GAAGATGCCAATGTTC | 51 | 39 |
| 912732 | 429 | 444 | 6003 | 6018 | TTAAGTTGAAGGATGG | 96 | 40 |
| 912733 | 432 | 447 | 6006 | 6021 | TGCTTAAGTTGAAGGA | 90 | 41 |
| 912734 | 478 | 493 | 6052 | 6067 | TGGACATTGGCCGGGA | 85 | 42 |
| 912735 | 479 | 494 | 6053 | 6068 | GTGGACATTGGCCGGG | 50 | 43 |
| 912736 | 484 | 499 | 6058 | 6073 | AGCTGGTGGACATTGG | 64 | 44 |
| 912737 | 528 | 543 | 6102 | 6117 | CATCAGACACTCTGGT | 5 | 45 |
| 912738 | 531 | 546 | 6105 | 6120 | CCCCATCAGACACTCT | 73 | 46 |
| 912739 | 552 | 567 | 6126 | 6141 | AGTCAGACACCAGAAC | 54 | 47 |
| 912755 | 693 | 708 | 11911 | 11926 | TGGCATCAATGAAGGG | 74 | 48 |
| 912756 | 698 | 713 | 11916 | 11931 | TGTTTTGGCATCAATG | 91 | 49 |
| 912757 | 746 | 761 | 11964 | 11979 | TTTAGGGCAGATGTCG | 89 | 50 |
| 912758 | 747 | 762 | 11965 | 11980 | CTTTAGGGCAGATGTC | 90 | 51 |
| 912759 | 795 | 810 | 12013 | 12028 | GTAGACTGAGCTTGGT | 98 | 52 |
| 912760 | 820 | 835 | 12038 | 12053 | AGGTAGAGGTTCCCTG | 0 | 53 |
| 912761 | 841 | 856 | 12059 | 12074 | GGGACAAAAGCTCTCG | 20 | 54 |
| 912762 | 873 | 888 | 13609 | 13624 | GGCATATCTCTCCCAG | 0 | 55 |
| 912763 | 874 | 889 | 13610 | 13625 | AGGCATATCTCTCCCA | 0 | 56 |
| 912764 | 886 | 901 | 13622 | 13637 | AAATATCCTCGAAGGC | 57 | 57 |
| 912765 | 888 | 903 | 13624 | 13639 | CCAAATATCCTCGAAG | 30 | 58 |
| 912766 | 889 | 904 | 13625 | 13640 | TCCAAATATCCTCGAA | 38 | 59 |
| 912767 | 894 | 909 | 13630 | 13645 | ATGCATCCAAATATCC | 58 | 60 |
| 912768 | 925 | 940 | N/A | N/A | TTGCAGATGCCCTTCT | 15 | 61 |
| 912769 | 968 | 983 | 16088 | 16103 | ATCCATCCCTTCTGAG | 34 | 62 |
| 912770 | 986 | 1001 | 16106 | 16121 | GGGCATGGCGACCTCA | 0 | 63 |
| 912771 | 1004 | 1019 | 16124 | 16139 | ACTCATGTTTGCCCAG | 67 | 64 |
| 912772 | 1068 | 1083 | 16188 | 16203 | GGTCTAGCAGCTCATC | 89 | 65 |
| 912773 | 1075 | 1090 | 16195 | 16210 | CGCAGGTGGTCTAGCA | 0 | 66 |
| 912774 | 1076 | 1091 | 16196 | 16211 | ACGCAGGTGGTCTAGC | 25 | 67 |
| 912775 | 1080 | 1095 | 16200 | 16215 | TGAGACGCAGGTGGTC | 50 | 68 |
| 912776 | 1086 | 1101 | 16206 | 16221 | GGATGCTGAGACGCAG | 67 | 69 |
| 912777 | 1172 | 1187 | 19012 | 19027 | GTATCCACCTTTGTCT | 78 | 70 |
| 912778 | 1178 | 1193 | 19018 | 19033 | GCTCATGTATCCACCT | 79 | 71 |
| 912779 | 1187 | 1202 | 19027 | 19042 | GCAAATCTTGCTCATG | 3 | 72 |
| 912780 | 1188 | 1203 | 19028 | 19043 | TGCAAATCTTGCTCAT | 13 | 73 |
| 912781 | 1189 | 1204 | 19029 | 19044 | TTGCAAATCTTGCTCA | 0 | 74 |
| 912782 | 1195 | 1210 | 19035 | 19050 | AGCAAGTTGCAAATCT | 77 | 75 |
| 912783 | 1199 | 1214 | 19039 | 19054 | GGGTAGCAAGTTGCAA | 74 | 76 |
| 912784 | 1205 | 1220 | 19045 | 19060 | CCTAATGGGTAGCAAG | 62 | 77 |
| 912785 | 1206 | 1221 | 19046 | 19061 | TCCTAATGGGTAGCAA | 79 | 78 |

TABLE 2
Inhibition of PNPLA3 mRNA by 3-10-3 cEt gapmers targeting SEQ ID NO: 1 and 2

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | PNPLA3 | SEQ |
| Compound | Start | Stop | Start | Stop | | % | ID |
| Number | Site | Site | Site | Site | Sequence (5′ to 3′) | Inhibition | NO |
| 912786 | 1207 | 1222 | 19047 | 19062 | ATCCTAATGGGTAGCA | 81 | 79 |
| 912787 | 1211 | 1226 | 19051 | 19066 | CATTATCCTAATGGGT | 46 | 80 |
| 912788 | 1212 | 1227 | 19052 | 19067 | ACATTATCCTAATGGG | 0 | 81 |
| 912789 | 1213 | 1228 | 19053 | 19068 | GACATTATCCTAATGG | 70 | 82 |
| 912790 | 1220 | 1235 | 19060 | 19075 | TACATAAGACATTATC | 34 | 83 |
| 912791 | 1224 | 1239 | 19064 | 19079 | GCATTACATAAGACAT | 86 | 84 |
| 912792 | 1245 | 1260 | 19085 | 19100 | CCACAGGCAGGGTACA | 76 | 85 |
| 912793 | 1246 | 1261 | 19086 | 19101 | TCCACAGGCAGGGTAC | 28 | 86 |
| 912794 | 1253 | 1268 | 19093 | 19108 | GGCAGATTCCACAGGC | 75 | 87 |
| 912795 | 1259 | 1274 | 19099 | 19114 | CGCAATGGCAGATTCC | 92 | 88 |
| 912796 | 1265 | 1280 | 19105 | 19120 | GACAATCGCAATGGCA | 64 | 89 |
| 912797 | 1266 | 1281 | 19106 | 19121 | GGACAATCGCAATGGC | 75 | 90 |
| 912798 | 1267 | 1282 | 19107 | 19122 | TGGACAATCGCAATGG | 73 | 91 |
| 912799 | 1285 | 1300 | 23690 | 23705 | AGCCATGTCACCAGTC | 67 | 92 |
| 912800 | 1289 | 1304 | 23694 | 23709 | TGGAAGCCATGTCACC | 24 | 93 |
| 912801 | 1290 | 1305 | 23695 | 23710 | CTGGAAGCCATGTCAC | 72 | 94 |
| 912802 | 1297 | 1312 | 23702 | 23717 | GGCATATCTGGAAGCC | 0 | 95 |
| 912803 | 1298 | 1313 | 23703 | 23718 | GGGCATATCTGGAAGC | 0 | 96 |
| 912804 | 1351 | 1366 | 23756 | 23771 | AGCACTCGAGTGAACA | 0 | 97 |
| 912805 | 1386 | 1401 | N/A | N/A | GCATTTGGGACCTGGA | 77 | 98 |
| 912806 | 1387 | 1402 | N/A | N/A | GGCATTTGGGACCTGG | 60 | 99 |
| 912807 | 1388 | 1403 | 25151 | 25166 | TGGCATTTGGGACCTG | 41 | 100 |
| 912808 | 1394 | 1409 | 25157 | 25172 | GCTCACTGGCATTTGG | 44 | 101 |
| 912809 | 1523 | 1538 | 25286 | 25301 | GTTCAGGCTGGACCTG | 49 | 102 |
| 912810 | 1547 | 1562 | 25310 | 25325 | AGGTACTTTATTGCCC | 11 | 103 |
| 912811 | 1550 | 1565 | 25313 | 25328 | AGCAGGTACTTTATTG | 64 | 104 |
| 912812 | 1653 | 1668 | 25416 | 25431 | AACTTTAGCACCTCTG | 91 | 105 |
| 912813 | 1655 | 1670 | 25418 | 25433 | GAAACTTTAGCACCTC | 88 | 106 |
| 912814 | 1656 | 1671 | 25419 | 25434 | GGAAACTTTAGCACCT | 53 | 107 |
| 912815 | 1669 | 1684 | 25432 | 25447 | CTGCACAAAGATGGGA | 80 | 108 |
| 912816 | 1671 | 1686 | 25434 | 25449 | AGCTGCACAAAGATGG | 45 | 109 |
| 912817 | 1685 | 1700 | 25448 | 25463 | AGCAATGCGGAGGTAG | 15 | 110 |
| 912818 | 1740 | 1755 | 25503 | 25518 | ACCAACTCAGCTCAGA | 85 | 111 |
| 912819 | 1741 | 1756 | 25504 | 25519 | AACCAACTCAGCTCAG | 79 | 112 |
| 912820 | 1757 | 1772 | 25520 | 25535 | TCCTAGCTTTTCATAA | 23 | 113 |
| 912821 | 1788 | 1803 | 25551 | 25566 | TGCTGGACCGCTGCAC | 0 | 114 |
| 912822 | 1796 | 1811 | 25559 | 25574 | GAGTTAAGTGCTGGAC | 93 | 115 |
| 912823 | 1802 | 1817 | 25565 | 25580 | GTATTAGAGTTAAGTG | 92 | 116 |
| 912824 | 1803 | 1818 | 25566 | 25581 | TGTATTAGAGTTAAGT | 79 | 117 |
| 912825 | 1806 | 1821 | 25569 | 25584 | TGATGTATTAGAGTTA | 92 | 118 |
| 912826 | 1808 | 1823 | 25571 | 25586 | GCTGATGTATTAGAGT | 80 | 119 |
| 912827 | 1821 | 1836 | 25584 | 25599 | TGAATTAACGCATGCT | 83 | 120 |
| 912828 | 1822 | 1837 | 25585 | 25600 | CTGAATTAACGCATGC | 78 | 121 |
| 912829 | 1870 | 1885 | 25633 | 25648 | AGTAAGGGACCCTCTG | 17 | 122 |
| 912830 | 1871 | 1886 | 25634 | 25649 | CAGTAAGGGACCCTCT | 28 | 123 |
| 912831 | 1872 | 1887 | 25635 | 25650 | TCAGTAAGGGACCCTC | 77 | 124 |
| 912832 | 1874 | 1889 | 25637 | 25652 | AGTCAGTAAGGGACCC | 51 | 125 |
| 912833 | 1893 | 1908 | 25656 | 25671 | ATTAATAGGGCCACGA | 80 | 126 |
| 912834 | 1895 | 1910 | 25658 | 25673 | CCATTAATAGGGCCAC | 90 | 127 |
| 912835 | 1896 | 1911 | 25659 | 25674 | ACCATTAATAGGGCCA | 81 | 128 |
| 912836 | 1906 | 1921 | 25669 | 25684 | GAACAGTCTGACCATT | 82 | 129 |
| 912837 | 1908 | 1923 | 25671 | 25686 | TGGAACAGTCTGACCA | 31 | 130 |
| 912838 | 1909 | 1924 | 25672 | 25687 | CTGGAACAGTCTGACC | 83 | 131 |
| 912839 | 1911 | 1926 | 25674 | 25689 | TGCTGGAACAGTCTGA | 72 | 132 |
| 912840 | 1916 | 1931 | 25679 | 25694 | CCTCATGCTGGAACAG | 83 | 133 |
| 912841 | 1928 | 1943 | 25691 | 25706 | TCATTCTAAGAACCTC | 96 | 134 |
| 912842 | 1945 | 1960 | 25708 | 25723 | ACCCATCCAAACACCT | 16 | 135 |
| 912843 | 1982 | 1997 | 25745 | 25760 | ACACATGGGCCAGCCT | 70 | 136 |
| 912844 | 1989 | 2004 | 25752 | 25767 | CAAGATCACACATGGG | 70 | 137 |
| 912845 | 2057 | 2072 | 25820 | 25835 | GGGACGAACTGCACCC | 0 | 138 |
| 912846 | 2098 | 2113 | 25861 | 25876 | TATCATCTTTGCAGAC | 81 | 139 |
| 912847 | 2116 | 2131 | 25879 | 25894 | GTTTTTAGTAGTCAAG | 91 | 140 |
| 912848 | 2117 | 2132 | 25880 | 25895 | CGTTTTTAGTAGTCAA | 91 | 141 |
| 912849 | 2145 | 2160 | 25908 | 25923 | TATCATCTTGTTACCC | 85 | 142 |
| 912850 | 2148 | 2163 | 25911 | 25926 | GATTATCATCTTGTTA | 70 | 143 |
| 912851 | 2150 | 2165 | 25913 | 25928 | TAGATTATCATCTTGT | 53 | 144 |
| 912852 | 2151 | 2166 | 25914 | 25929 | GTAGATTATCATCTTG | 80 | 145 |
| 912853 | 2152 | 2167 | 25915 | 25930 | AGTAGATTATCATCTT | 84 | 146 |
| 912854 | 2175 | 2190 | 25938 | 25953 | GTGAAAAAGGTGTTCT | 77 | 147 |
| 912855 | 2182 | 2197 | 25945 | 25960 | TAGTTAGGTGAAAAAG | 92 | 148 |
| 912856 | 2188 | 2203 | 25951 | 25966 | TTATTTTAGTTAGGTG | 88 | 149 |
| 912857 | 2190 | 2205 | 25953 | 25968 | CATTATTTTAGTTAGG | 86 | 150 |
| 912858 | 2273 | 2288 | 26036 | 26051 | CTACTAACATCTCACT | 55 | 151 |
| 912859 | 2274 | 2289 | 26037 | 26052 | TCTACTAACATCTCAC | 89 | 152 |
| 912860 | 2278 | 2293 | 26041 | 26056 | TTATTCTACTAACATC | 27 | 153 |
| 912861 | 2280 | 2295 | 26043 | 26058 | GCTTATTCTACTAACA | 79 | 154 |
| 912862 | 2281 | 2296 | 26044 | 26059 | GGCTTATTCTACTAAC | 81 | 155 |
| 912863 | 2632 | 2647 | 26395 | 26410 | GGTGAATGCCCTGCAC | 41 | 156 |

TABLE 3
Inhibition of PNPLA3 mRNA by 3-10-3 cEt gapmers targeting SEQ ID NO: 1 and 2

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | PNPLA3 | SEQ |
| Compound | Start | Stop | Start | Stop | | % | ID |
| Number | Site | Site | Site | Site | Sequence (5′ to 3′) | Inhibition | NO |
| 912864 | 2703 | 2718 | 26466 | 26481 | TTCAAGTTGTGTGCTC | 90 | 157 |
| 912865 | 2755 | 2770 | 26518 | 26533 | GGGAGAAACTCACTGA | 37 | 158 |
| 912866 | N/A | N/A | 4416 | 4431 | TGCTACTTGCCCCAGC | 2 | 159 |
| 912867 | N/A | N/A | 4421 | 4436 | CACAATGCTACTTGCC | 87 | 160 |
| 912868 | N/A | N/A | 4584 | 4599 | CCCAATGGCAGGGCTT | 58 | 161 |
| 912869 | N/A | N/A | 4592 | 4607 | TGCTCCTACCCAATGG | 46 | 162 |
| 912870 | N/A | N/A | 4766 | 4781 | GACTTTTATTGTTGCT | 95 | 163 |
| 912871 | N/A | N/A | 4883 | 4898 | TTCTATACCAGAGTGA | 89 | 164 |
| 912872 | N/A | N/A | 4884 | 4899 | TTTCTATACCAGAGTG | 89 | 165 |
| 912873 | N/A | N/A | 5405 | 5420 | GTAGATGGCCTTAATG | 83 | 166 |
| 912876 | N/A | N/A | 6155 | 6170 | TACATCCACGACTTCG | 94 | 167 |
| 912877 | N/A | N/A | 6156 | 6171 | TTACATCCACGACTTC | 76 | 168 |
| 912880 | N/A | N/A | 6606 | 6621 | GGAACATTCAGGGTTT | 13 | 169 |
| 912881 | N/A | N/A | 6834 | 6849 | ATTACTTGGGTGCAGG | 55 | 170 |
| 912884 | N/A | N/A | 6838 | 6853 | GCAGATTACTTGGGTG | 45 | 171 |
| 912885 | N/A | N/A | 6931 | 6946 | TGCAGGACAGGTTCCT | 30 | 172 |
| 912888 | N/A | N/A | 7549 | 7564 | CACACTGGGTCACCAC | 55 | 173 |
| 912889 | N/A | N/A | 7552 | 7567 | AGTCACACTGGGTCAC | 61 | 174 |
| 912928 | N/A | N/A | 12273 | 12288 | GGTATATGTTCCCAGG | 87 | 175 |
| 912929 | N/A | N/A | 12314 | 12329 | TATAACCACAGCCTGG | 29 | 176 |
| 912932 | N/A | N/A | 12321 | 12336 | CTGACTATATAACCAC | 81 | 177 |
| 912933 | N/A | N/A | 12666 | 12681 | ATCTTAGTGGCTGGGT | 91 | 178 |
| 912936 | N/A | N/A | 12767 | 12782 | CTTACTATGGTAGAGT | 88 | 179 |
| 912937 | N/A | N/A | 12768 | 12783 | TCTTACTATGGTAGAG | 74 | 180 |
| 912940 | N/A | N/A | 12835 | 12850 | TGCATTGCATAGCCTT | 97 | 181 |
| 912941 | N/A | N/A | 12836 | 12851 | TTGCATTGCATAGCCT | 96 | 182 |
| 912944 | N/A | N/A | 12907 | 12922 | TGCTTATAAAGCACAC | 61 | 183 |
| 912945 | N/A | N/A | 12988 | 13003 | GGAATAAGCCTCCACC | 14 | 184 |
| 912948 | N/A | N/A | 14055 | 14070 | GAAATCTGATTGCTTC | 59 | 185 |
| 912949 | N/A | N/A | 14393 | 14408 | TACTTATCTGCTCACT | 66 | 186 |
| 912952 | N/A | N/A | 14673 | 14688 | TCTCTTAGTGTCCCCA | 90 | 187 |
| | | | 14707 | 14722 | | | |
| 912953 | N/A | N/A | 14674 | 14689 | ATCTCTTAGTGTCCCC | 92 | 188 |
| | | | 14708 | 14723 | | | |
| 912956 | N/A | N/A | 15284 | 15299 | TCACATTCATGCTTGC | 82 | 189 |
| 912957 | N/A | N/A | 15291 | 15306 | GATAACCTCACATTCA | 0 | 190 |
| 912960 | N/A | N/A | 15712 | 15727 | GAGCTAGGTGCTTCAC | 6 | 191 |
| 912961 | N/A | N/A | 15753 | 15768 | ATAACAACTGAACCAC | 85 | 192 |
| 912964 | N/A | N/A | 15937 | 15952 | GTTATTAGCCAAATGC | 92 | 193 |
| 912965 | N/A | N/A | 16468 | 16483 | GGAGACTTGGCAAGGT | 87 | 194 |
| 912968 | N/A | N/A | 16960 | 16975 | ATTCATGACAGCCCTT | 46 | 195 |
| 912969 | N/A | N/A | 17128 | 17143 | ATCGATTTTTCAGAGT | 9 | 196 |
| 912972 | N/A | N/A | 17134 | 17149 | ACAAACATCGATTTTT | 52 | 197 |
| 912973 | N/A | N/A | 17769 | 17784 | CTCTTTAATGACCTCG | 90 | 198 |
| 912976 | N/A | N/A | 18865 | 18880 | GTCAGAGGCACTCACA | 25 | 199 |
| 912977 | N/A | N/A | 18959 | 18974 | AGCTATTATCTCCCAC | 0 | 200 |
| 912980 | N/A | N/A | 19315 | 19330 | AGTTTCTGGGCTTGCA | 90 | 201 |
| 912981 | N/A | N/A | 19382 | 19397 | GGCAATCACAAGAGAC | 73 | 202 |
| 912984 | N/A | N/A | 20286 | 20301 | AGAGGAAGCCCAATCA | 79 | 203 |
| | | | 20316 | 20331 | | | |
| 912985 | N/A | N/A | 20287 | 20302 | CAGAGGAAGCCCAATC | 93 | 204 |
| | | | 20317 | 20332 | | | |
| 912988 | N/A | N/A | 20658 | 20673 | TAGAAATTGCAGTGCC | 92 | 205 |
| 912989 | N/A | N/A | 20731 | 20746 | TCCTATCCATATATTG | 55 | 206 |
| 912992 | N/A | N/A | 21408 | 21423 | GCAATTCTAGACATGG | 88 | 207 |
| 912993 | N/A | N/A | 21558 | 21573 | AGGACTTACACCAAGA | 86 | 208 |
| 912996 | N/A | N/A | 21936 | 21951 | TTCCTAATAAGAGCCC | 24 | 209 |
| 912997 | N/A | N/A | 21946 | 21961 | GTCAAACATCTTCCTA | 66 | 210 |
| 913000 | N/A | N/A | 22077 | 22092 | AAAACTGTAGGATAGG | 47 | 211 |
| 913001 | N/A | N/A | 22162 | 22177 | GTTACATCCATAAAAC | 0 | 212 |
| 913004 | N/A | N/A | 22169 | 22184 | AGAGAATGTTACATCC | 62 | 213 |
| 913008 | N/A | N/A | 23083 | 23098 | AAAGATTAATCAGGGC | 61 | 214 |
| 913012 | N/A | N/A | 23788 | 23803 | GTATTTACCTGGAGGC | 0 | 215 |
| 913016 | N/A | N/A | 24426 | 24441 | GGCCTATGATTTTCAG | 0 | 216 |

TABLE 4
Inhibition of PNPLA3 mRNA by 3-10-3 cEt gapmers targeting SEQ ID NO: 1 and 2

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | PNPLA3 | SEQ |
| Compound | Start | Stop | Start | Stop | | % | ID |
| Number | Site | Site | Site | Site | Sequence (5′ to 3′) | Inhibition | NO |
| 912874 | N/A | N/A | 5869 | 5884 | ATACTTTTGGCAAGGC | 96 | 217 |
| 912875 | N/A | N/A | 5870 | 5885 | AATACTTTTGGCAAGG | 91 | 218 |
| 912878 | N/A | N/A | 6159 | 6174 | TGCTTACATCCACGAC | 12 | 219 |
| 912879 | N/A | N/A | 6296 | 6311 | CATCATGTTGGTCTCG | 54 | 220 |
| 912882 | N/A | N/A | 6835 | 6850 | GATTACTTGGGTGCAG | 39 | 221 |
| 912883 | N/A | N/A | 6837 | 6852 | CAGATTACTTGGGTGC | 69 | 222 |
| 912886 | N/A | N/A | 7083 | 7098 | TTTAATGGTGTTTTGG | 87 | 223 |
| 912887 | N/A | N/A | 7478 | 7493 | TCAAATGCCGGTATTC | 52 | 224 |
| 912890 | N/A | N/A | 7587 | 7602 | GTGAACTTCAACTTCC | 56 | 225 |
| 912930 | N/A | N/A | 12317 | 12332 | CTATATAACCACAGCC | 77 | 226 |
| 912931 | N/A | N/A | 12319 | 12334 | GACTATATAACCACAG | 92 | 227 |
| 912934 | N/A | N/A | 12670 | 12685 | AATCATCTTAGTGGCT | 91 | 228 |
| 912935 | N/A | N/A | 12765 | 12780 | TACTATGGTAGAGTGG | 80 | 229 |
| 912938 | N/A | N/A | 12786 | 12801 | GTACATGGTCTGCAAA | 84 | 230 |
| 912939 | N/A | N/A | 12787 | 12802 | TGTACATGGTCTGCAA | 57 | 231 |
| 912942 | N/A | N/A | 12843 | 12858 | GCATGCATTGCATTGC | 16 | 232 |
| 912943 | N/A | N/A | 12885 | 12900 | ACCAATCCTGTTAGAC | 93 | 233 |
| 912946 | N/A | N/A | 13557 | 13572 | GGAGACACCAAGCACC | 42 | 234 |
| 912947 | N/A | N/A | 13751 | 13766 | GCACTAAGTGTTAGAA | 79 | 235 |
| 912950 | N/A | N/A | 14396 | 14411 | GCTTACTTATCTGCTC | 0 | 236 |
| 912951 | N/A | N/A | 14501 | 14516 | GGAGATCCATCCTGCA | 0 | 237 |
| 912954 | N/A | N/A | 14675 | 14690 | CATCTCTTAGTGTCCC | 92 | 238 |
| | | | 14709 | 14724 | | | |
| 912955 | N/A | N/A | 15122 | 15137 | TCCTAATGTCCTCAAC | 9 | 239 |
| 912958 | N/A | N/A | 15293 | 15308 | AAGATAACCTCACATT | 33 | 240 |
| 912959 | N/A | N/A | 15294 | 15309 | CAAGATAACCTCACAT | 22 | 241 |
| 912962 | N/A | N/A | 15754 | 15769 | TATAACAACTGAACCA | 82 | 242 |
| 912963 | N/A | N/A | 15856 | 15871 | GCTTTAAAGCAGGACA | 8 | 243 |
| 912966 | N/A | N/A | 16774 | 16789 | AAAATTGTGGGTTTAG | 68 | 244 |
| 912967 | N/A | N/A | 16850 | 16865 | ATCATTTGGACCATAG | 81 | 245 |
| 912970 | N/A | N/A | 17130 | 17145 | ACATCGATTTTTCAGA | 83 | 246 |
| 912971 | N/A | N/A | 17133 | 17148 | CAAACATCGATTTTTC | 62 | 247 |
| 912974 | N/A | N/A | 17843 | 17858 | GCTTTACAAGCTGGTC | 0 | 248 |
| 912975 | N/A | N/A | 17879 | 17894 | ATCTATGTTCTCCTAG | 0 | 249 |
| 912978 | N/A | N/A | 19125 | 19140 | ACCTAAAATGCTCACC | 0 | 250 |
| 912979 | N/A | N/A | 19198 | 19213 | CCAGACTACATGCCAC | 79 | 251 |
| 912982 | N/A | N/A | 19446 | 19461 | TCTACTAGGCATCTCT | 63 | 252 |
| 912983 | N/A | N/A | 19447 | 19462 | TTCTACTAGGCATCTC | 42 | 253 |
| 912986 | N/A | N/A | 20288 | 20303 | TCAGAGGAAGCCCAAT | 92 | 254 |
| | | | 20318 | 20333 | | | |
| 912987 | N/A | N/A | 20656 | 20671 | GAAATTGCAGTGCCCT | 92 | 255 |
| 912990 | N/A | N/A | 21393 | 21408 | GCCAACCTATCACTGA | 60 | 256 |
| 912991 | N/A | N/A | 21400 | 21415 | AGACATGGCCAACCTA | 32 | 257 |
| 912994 | N/A | N/A | 21565 | 21580 | TGAAATAAGGACTTAC | 67 | 258 |
| 912995 | N/A | N/A | 21934 | 21949 | CCTAATAAGAGCCCCA | 31 | 259 |
| 912998 | N/A | N/A | 22041 | 22056 | GAAATCTGTCAGAGCA | 33 | 260 |
| 912999 | N/A | N/A | 22072 | 22087 | TGTAGGATAGGACTAG | 0 | 261 |
| 913002 | N/A | N/A | 22166 | 22181 | GAATGTTACATCCATA | 53 | 262 |
| 913003 | N/A | N/A | 22168 | 22183 | GAGAATGTTACATCCA | 80 | 263 |
| 913005 | N/A | N/A | 22605 | 22620 | GTGATAAATCTGCAAG | 70 | 264 |
| 913006 | N/A | N/A | 23081 | 23096 | AGATTAATCAGGGCCA | 8 | 265 |
| 913007 | N/A | N/A | 23082 | 23097 | AAGATTAATCAGGGCC | 30 | 266 |
| 913009 | N/A | N/A | 23325 | 23340 | GGTCACATGTGAGCCC | 0 | 267 |
| 913010 | N/A | N/A | 23496 | 23511 | CACTTCTGGTTCAAGA | 13 | 268 |
| 913011 | N/A | N/A | 23580 | 23595 | CCAATCTGATGACTTC | 80 | 269 |
| 913013 | N/A | N/A | 23790 | 23805 | AAGTATTTACCTGGAG | 0 | 270 |
| 913014 | N/A | N/A | 24028 | 24043 | CACTCAAAGAGACTCA | 65 | 271 |
| 913015 | N/A | N/A | 24425 | 24440 | GCCTATGATTTTCAGG | 0 | 272 |
| 913017 | N/A | N/A | 24633 | 24648 | CACTACTGCCCTCTTC | 50 | 273 |
| 913018 | N/A | N/A | 24983 | 24998 | TGCTGGGCTGATGTCA | 0 | 274 |
| 913019 | N/A | N/A | 25150 | 25165 | GGCATTTGGGACCTGA | 67 | 275 |

TABLE 5
Inhibition of PNPLA3 mRNA by 3-10-3 cEt gapmers targeting SEQ ID NO: 1 and 2

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | PNPLA3 | SEQ |
| Compound | Start | Stop | Start | Stop | | % | ID |
| Number | Site | Site | Site | Site | Sequence (5′ to 3′) | Inhibition | NO |
| 915343 | 1 | 16 | 2739 | 2754 | GCCCCCCTCGGACCAT | 0 | 276 |
| 915363 | 45 | 60 | 2783 | 2798 | CCTCAGTGTCTCGGCC | 0 | 277 |
| 915383 | 107 | 122 | 2845 | 2860 | AATCGGCTCGGGTCCT | 29 | 278 |
| 915403 | 190 | 205 | 2928 | 2943 | GACAAGCTCCAGCCGC | 64 | 279 |
| 915423 | 249 | 264 | 2987 | 3002 | CGCTCAGGCAGCGGGT | 0 | 280 |
| 915443 | 347 | 362 | N/A | N/A | CTCCAGCGGGATACCG | 6 | 281 |
| 915463 | 386 | 401 | 5960 | 5975 | GGCCTTCCGCACAAGA | 0 | 282 |
| 915483 | 416 | 431 | 5990 | 6005 | TGGATGGAAGATGCCA | 28 | 283 |
| 915503 | 452 | 467 | 6026 | 6041 | GAGACCCTGTCGGAGG | 45 | 284 |
| 915523 | 488 | 503 | 6062 | 6077 | GATGAGCTGGTGGACA | 70 | 285 |
| 915543 | 510 | 525 | 6084 | 6099 | GAGAGATGCCTATTTT | 92 | 286 |
| 915563 | 559 | 574 | 6133 | 6148 | GACCGAAAGTCAGACA | 7 | 287 |
| 915603 | 697 | 712 | 11915 | 11930 | GTTTTGGCATCAATGA | 94 | 288 |
| 915623 | 754 | 769 | 11972 | 11987 | GACTTGACTTTAGGGC | 98 | 289 |
| 915643 | 827 | 842 | 12045 | 12060 | CGAGAGAAGGTAGAGG | 97 | 290 |
| 915663 | 879 | 894 | 13615 | 13630 | CTCGAAGGCATATCTC | 66 | 291 |
| 915683 | 932 | 947 | 16052 | 16067 | GGGCCTGTTGCAGATG | 0 | 292 |
| 915703 | 985 | 1000 | 16105 | 16120 | GGCATGGCGACCTCAG | 6 | 293 |
| 915723 | 1037 | 1052 | 16157 | 16172 | AGCCAAGGCAGCCGAC | 0 | 294 |
| 915743 | 1132 | 1147 | 16252 | 16267 | GCGAGCCTGGGCGAGA | 0 | 295 |
| 915763 | 1177 | 1192 | 19017 | 19032 | CTCATGTATCCACCTT | 88 | 296 |
| 915783 | 1229 | 1244 | 19069 | 19084 | GGGCAGCATTACATAA | 73 | 297 |
| 915803 | 1286 | 1301 | 23691 | 23706 | AAGCCATGTCACCAGT | 34 | 298 |
| 915823 | 1348 | 1363 | 23753 | 23768 | ACTCGAGTGAACACCT | 12 | 299 |
| 915843 | 1405 | 1420 | 25168 | 25183 | GCCTGTTGGCTGCTCA | 1 | 300 |
| 915863 | 1473 | 1488 | 25236 | 25251 | CTGCTGGACAGCCCTT | 0 | 301 |
| 915883 | 1542 | 1557 | 25305 | 25320 | CTTTATTGCCCAAGAA | 72 | 302 |
| 915903 | 1601 | 1616 | 25364 | 25379 | CAGACTCTTCTCTAGT | 49 | 303 |
| 915923 | 1633 | 1648 | 25396 | 25411 | AATCTGCTAGACTCGC | 88 | 304 |
| 915943 | 1686 | 1701 | 25449 | 25464 | CAGCAATGCGGAGGTA | 80 | 305 |
| 915963 | 1768 | 1783 | 25531 | 25546 | GAAAGGTTGCTTCCTA | 84 | 306 |
| 915983 | 1789 | 1804 | 25552 | 25567 | GTGCTGGACCGCTGCA | 11 | 307 |
| 916003 | 1815 | 1830 | 25578 | 25593 | AACGCATGCTGATGTA | 69 | 308 |
| 916023 | 1848 | 1863 | 25611 | 25626 | GCTTCCTGGTGTCATT | 81 | 309 |
| 916043 | 1884 | 1899 | 25647 | 25662 | GCCACGAAACAGTCAG | 67 | 310 |
| 916063 | 1913 | 1928 | 25676 | 25691 | CATGCTGGAACAGTCT | 20 | 311 |
| 916083 | 1954 | 1969 | 25717 | 25732 | AAGGCCCCCACCCATC | 0 | 312 |
| 916103 | 1977 | 1992 | 25740 | 25755 | TGGGCCAGCCTACCCC | 0 | 313 |
| 916123 | 2026 | 2041 | 25789 | 25804 | GGAAGTGGGATCATGC | 55 | 314 |
| 916142 | 2100 | 2115 | 25863 | 25878 | GTTATCATCTTTGCAG | 57 | 315 |
| 916162 | 2139 | 2154 | 25902 | 25917 | CTTGTTACCCCCGCCA | 84 | 316 |
| 916182 | 2264 | 2279 | 26027 | 26042 | TCTCACTGATTCACAT | 83 | 317 |
| 916202 | 2624 | 2639 | 26387 | 26402 | CCCTGCACACTAGATT | 55 | 318 |
| 916222 | 2677 | 2692 | 26440 | 26455 | GAGGCGGAAGCTCCTG | 0 | 319 |
| 916242 | 2707 | 2722 | 26470 | 26485 | CAGGTTCAAGTTGTGT | 83 | 320 |
| 916282 | N/A | N/A | 4225 | 4240 | AAATGTACGGAATCTC | 79 | 321 |
| 916302 | N/A | N/A | 4822 | 4837 | GTGTAAACATTTGTCC | 74 | 322 |
| 916322 | N/A | N/A | 5414 | 5429 | AGCTTTGGTGTAGATG | 49 | 323 |
| 916342 | N/A | N/A | 5801 | 5816 | TACTATGGGAGCCACA | 42 | 324 |
| 916362 | N/A | N/A | 6866 | 6881 | TGAAATTGTAACTGCC | 70 | 325 |
| 916382 | N/A | N/A | 7492 | 7507 | TAGATCGGTGCTGTTC | 27 | 326 |
| 916402 | N/A | N/A | 7785 | 7800 | GTTATAGGCGAGAGCA | 0 | 327 |
| 916562 | N/A | N/A | 12316 | 12331 | TATATAACCACAGCCT | 58 | 328 |
| 916582 | N/A | N/A | 12932 | 12947 | ATAAGAGCTGTCTCCT | 94 | 329 |
| 916602 | N/A | N/A | 13703 | 13718 | CTAGTAAATGCTTGTC | 96 | 330 |
| 916622 | N/A | N/A | 14177 | 14192 | CTAATATTTCTACAGC | 0 | 331 |
| 916642 | N/A | N/A | 14672 | 14687 | CTCTTAGTGTCCCCAT | 95 | 332 |
| 916662 | N/A | N/A | 15542 | 15557 | TTCCATCACAAGGCCT | 50 | 333 |
| 916682 | N/A | N/A | 16317 | 16332 | TCCATAATGCACAAGA | 71 | 334 |
| 916702 | N/A | N/A | 17223 | 17238 | TGTAGCTGGTTTGTGG | 88 | 335 |
| 916722 | N/A | N/A | 18223 | 18238 | AACAGCTACATCAGGC | 44 | 336 |
| 916742 | N/A | N/A | 19249 | 19264 | GGCATTGCACATAGAC | 74 | 337 |
| 916761 | N/A | N/A | 20410 | 20425 | GTAAGCAATGCAGCCA | 88 | 338 |
| 916781 | N/A | N/A | 20659 | 20674 | TTAGAAATTGCAGTGC | 91 | 339 |
| 916801 | N/A | N/A | 20989 | 21004 | AGGTATTAAACTGCCA | 25 | 340 |
| 916821 | N/A | N/A | 21506 | 21521 | GTCCTAAGAGCACTCA | 57 | 341 |
| 916841 | N/A | N/A | 22603 | 22618 | GATAAATCTGCAAGAG | 49 | 342 |
| 916861 | N/A | N/A | 23472 | 23487 | GGGACTTACACTGAAA | 66 | 343 |
| 916881 | N/A | N/A | 24314 | 24329 | GTCAACGCAGACTGCT | 33 | 344 |

TABLE 6
Inhibition of PNPLA3 mRNA by 3-10-3 cEt gapmers targeting SEQ ID NO: 1 and 2

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | PNPLA3 | SEQ |
| Compound | Start | Stop | Start | Stop | | % | ID |
| Number | Site | Site | Site | Site | Sequence (5′ to 3′) | Inhibition | NO |
| 915344 | 2 | 17 | 2740 | 2755 | CGCCCCCCTCGGACCA | 0 | 345 |
| 915364 | 46 | 61 | 2784 | 2799 | GCCTCAGTGTCTCGGC | 0 | 346 |
| 915384 | 108 | 123 | 2846 | 2861 | GAATCGGCTCGGGTCC | 49 | 347 |
| 915404 | 191 | 206 | 2929 | 2944 | GGACAAGCTCCAGCCG | 9 | 348 |
| 915424 | 250 | 265 | 2988 | 3003 | TCGCTCAGGCAGCGGG | 0 | 349 |
| 915444 | 348 | 363 | N/A | N/A | GCTCCAGCGGGATACC | 0 | 350 |
| 915464 | 387 | 402 | 5961 | 5976 | TGGCCTTCCGCACAAG | 0 | 351 |
| 915484 | 428 | 443 | 6002 | 6017 | TAAGTTGAAGGATGGA | 96 | 352 |
| 915504 | 453 | 468 | 6027 | 6042 | AGAGACCCTGTCGGAG | 80 | 353 |
| 915524 | 489 | 504 | 6063 | 6078 | AGATGAGCTGGTGGAC | 81 | 354 |
| 915544 | 512 | 527 | 6086 | 6101 | AAGAGAGATGCCTATT | 77 | 355 |
| 915564 | 560 | 575 | 6134 | 6149 | GGACCGAAAGTCAGAC | 0 | 356 |
| 915604 | 700 | 715 | 11918 | 11933 | GTTGTTTTGGCATCAA | 91 | 357 |
| 915624 | 755 | 770 | 11973 | 11988 | GGACTTGACTTTAGGG | 81 | 358 |
| 915644 | 828 | 843 | 12046 | 12061 | TCGAGAGAAGGTAGAG | 24 | 359 |
| 915664 | 880 | 895 | 13616 | 13631 | CCTCGAAGGCATATCT | 41 | 360 |
| 915684 | 952 | 967 | 16072 | 16087 | GATGACTTCAGGCCTG | 0 | 361 |
| 915704 | 987 | 1002 | 16107 | 16122 | TGGGCATGGCGACCTC | 0 | 362 |
| 915724 | 1038 | 1053 | 16158 | 16173 | CAGCCAAGGCAGCCGA | 0 | 363 |
| 915744 | 1133 | 1148 | 16253 | 16268 | AGCGAGCCTGGGCGAG | 0 | 364 |
| 915764 | 1179 | 1194 | 19019 | 19034 | TGCTCATGTATCCACC | 56 | 365 |
| 915784 | 1230 | 1245 | 19070 | 19085 | AGGGCAGCATTACATA | 69 | 366 |
| 915804 | 1293 | 1308 | 23698 | 23713 | TATCTGGAAGCCATGT | 6 | 367 |
| 915824 | 1349 | 1364 | 23754 | 23769 | CACTCGAGTGAACACC | 0 | 368 |
| 915844 | 1406 | 1421 | 25169 | 25184 | GGCCTGTTGGCTGCTC | 0 | 369 |
| 915864 | 1477 | 1492 | 25240 | 25255 | GTCTCTGCTGGACAGC | 0 | 370 |
| 915884 | 1545 | 1560 | 25308 | 25323 | GTACTTTATTGCCCAA | 73 | 371 |
| 915904 | 1607 | 1622 | 25370 | 25385 | GACTCACAGACTCTTC | 92 | 372 |
| 915924 | 1634 | 1649 | 25397 | 25412 | GAATCTGCTAGACTCG | 65 | 373 |
| 915944 | 1687 | 1702 | 25450 | 25465 | ACAGCAATGCGGAGGT | 83 | 374 |
| 915964 | 1769 | 1784 | 25532 | 25547 | CGAAAGGTTGCTTCCT | 79 | 375 |
| 915984 | 1790 | 1805 | 25553 | 25568 | AGTGCTGGACCGCTGC | 38 | 376 |
| 916004 | 1816 | 1831 | 25579 | 25594 | TAACGCATGCTGATGT | 79 | 377 |
| 916024 | 1849 | 1864 | 25612 | 25627 | GGCTTCCTGGTGTCAT | 73 | 378 |
| 916044 | 1885 | 1900 | 25648 | 25663 | GGCCACGAAACAGTCA | 40 | 379 |
| 916064 | 1914 | 1929 | 25677 | 25692 | TCATGCTGGAACAGTC | 80 | 380 |
| 916084 | 1958 | 1973 | 25721 | 25736 | TCACAAGGCCCCCACC | 35 | 381 |
| 916104 | 1978 | 1993 | 25741 | 25756 | ATGGGCCAGCCTACCC | 0 | 382 |
| 916124 | 2053 | 2068 | 25816 | 25831 | CGAACTGCACCCCTTC | 38 | 383 |
| 916143 | 2101 | 2116 | 25864 | 25879 | GGTTATCATCTTTGCA | 81 | 384 |
| 916163 | 2140 | 2155 | 25903 | 25918 | TCTTGTTACCCCCGCC | 84 | 385 |
| 916183 | 2265 | 2280 | 26028 | 26043 | ATCTCACTGATTCACA | 86 | 386 |
| 916203 | 2625 | 2640 | 26388 | 26403 | GCCCTGCACACTAGAT | 65 | 387 |
| 916223 | 2678 | 2693 | 26441 | 26456 | GGAGGCGGAAGCTCCT | 0 | 388 |
| 916243 | 2709 | 2724 | 26472 | 26487 | GCCAGGTTCAAGTTGT | 62 | 389 |
| 916283 | N/A | N/A | 4226 | 4241 | CAAATGTACGGAATCT | 52 | 390 |
| 916303 | N/A | N/A | 4864 | 4879 | TACTTTAGGCTCCTGG | 90 | 391 |
| 916323 | N/A | N/A | 5422 | 5437 | AGCATTAGAGCTTTGG | 75 | 392 |
| 916343 | N/A | N/A | 5803 | 5818 | TCTACTATGGGAGCCA | 89 | 393 |
| 916363 | N/A | N/A | 6927 | 6942 | GGACAGGTTCCTTGGA | 0 | 394 |
| 916383 | N/A | N/A | 7493 | 7508 | CTAGATCGGTGCTGTT | 14 | 395 |
| 916403 | N/A | N/A | 7786 | 7801 | AGTTATAGGCGAGAGC | 0 | 396 |
| 916563 | N/A | N/A | 12318 | 12333 | ACTATATAACCACAGC | 90 | 397 |
| 916583 | N/A | N/A | 12936 | 12951 | GACAATAAGAGCTGTC | 0 | 398 |
| 916603 | N/A | N/A | 13704 | 13719 | GCTAGTAAATGCTTGT | 73 | 399 |
| 916623 | N/A | N/A | 14231 | 14246 | CCAACTTTTAGTATTA | 92 | 400 |
| 916643 | N/A | N/A | 14678 | 14693 | AGCCATCTCTTAGTGT | 50 | 401 |
| 916663 | N/A | N/A | 15566 | 15581 | TCTGATGTCGAAGAGG | 68 | 402 |
| 916683 | N/A | N/A | 16341 | 16356 | TCCCATGTGGCAGTAC | 0 | 403 |
| 916703 | N/A | N/A | 17239 | 17254 | TCCAAATGCCCAACTC | 37 | 404 |
| 916723 | N/A | N/A | 18241 | 18256 | GCAAATAATGTGCACA | 22 | 405 |
| 916743 | N/A | N/A | 19250 | 19265 | GGGCATTGCACATAGA | 59 | 406 |
| 916762 | N/A | N/A | 20413 | 20428 | GTAGTAAGCAATGCAG | 69 | 407 |
| 916782 | N/A | N/A | 20660 | 20675 | CTTAGAAATTGCAGTG | 91 | 408 |
| 916802 | N/A | N/A | 21002 | 21017 | ATTTTAACAGCTCAGG | 95 | 409 |
| 916822 | N/A | N/A | 21540 | 21555 | TATGACATTTCAGAGT | 88 | 410 |
| 916842 | N/A | N/A | 22629 | 22644 | AGTACAAGCGCAGCCT | 14 | 411 |
| 916862 | N/A | N/A | 23538 | 23553 | ACAAGGACAAGCCCAC | 37 | 412 |
| 916882 | N/A | N/A | 24339 | 24354 | GAAGTAGCGGCATCCC | 68 | 413 |

TABLE 7
Inhibition of PNPLA3 mRNA by 3-10-3 cEt gapmers targeting SEQ ID NO: 1 and 2

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | PNPLA3 | SEQ |
| Compound | Start | Stop | Start | Stop | Sequence | % | ID |
| Number | Site | Site | Site | Site | (5′ to 3′) | Inhibition | NO |
| 915345 | 3 | 18 | 2741 | 2756 | CCGCCCCCCTCGGACC | 0 | 414 |
| 915365 | 47 | 62 | 2785 | 2800 | TGCCTCAGTGTCTCGG | 0 | 415 |
| 915385 | 109 | 124 | 2847 | 2862 | GGAATCGGCTCGGGTC | 72 | 416 |
| 915405 | 193 | 208 | 2931 | 2946 | AAGGACAAGCTCCAGC | 41 | 417 |
| 915425 | 251 | 266 | 2989 | 3004 | CTCGCTCAGGCAGCGG | 0 | 418 |
| 915445 | 349 | 364 | N/A | N/A | TGCTCCAGCGGGATAC | 0 | 419 |
| 915465 | 388 | 403 | 5962 | 5977 | CTGGCCTTCCGCACAA | 16 | 420 |
| 915485 | 430 | 445 | 6004 | 6019 | CTTAAGTTGAAGGATG | 27 | 421 |
| 915505 | 454 | 469 | 6028 | 6043 | CAGAGACCCTGTCGGA | 72 | 422 |
| 915525 | 492 | 507 | 6066 | 6081 | CGGAGATGAGCTGGTG | 92 | 423 |
| 915545 | 513 | 528 | 6087 | 6102 | TAAGAGAGATGCCTAT | 57 | 424 |
| 915565 | 561 | 576 | 6135 | 6150 | TGGACCGAAAGTCAGA | 0 | 425 |
| 915605 | 701 | 716 | 11919 | 11934 | GGTTGTTTTGGCATCA | 97 | 426 |
| 915625 | 756 | 771 | 11974 | 11989 | TGGACTTGACTTTAGG | 93 | 427 |
| 915645 | 829 | 844 | 12047 | 12062 | CTCGAGAGAAGGTAGA | 0 | 428 |
| 915665 | 881 | 896 | 13617 | 13632 | TCCTCGAAGGCATATC | 0 | 429 |
| 915685 | 953 | 968 | 16073 | 16088 | GGATGACTTCAGGCCT | 0 | 430 |
| 915705 | 988 | 1003 | 16108 | 16123 | CTGGGCATGGCGACCT | 0 | 431 |
| 915725 | 1039 | 1054 | 16159 | 16174 | ACAGCCAAGGCAGCCG | 0 | 432 |
| 915745 | 1134 | 1149 | 16254 | 16269 | TAGCGAGCCTGGGCGA | 0 | 433 |
| 915765 | 1193 | 1208 | 19033 | 19048 | CAAGTTGCAAATCTTG | 0 | 434 |
| 915785 | 1231 | 1246 | 19071 | 19086 | CAGGGCAGCATTACAT | 74 | 435 |
| 915805 | 1300 | 1315 | 23705 | 23720 | TCGGGCATATCTGGAA | 21 | 436 |
| 915825 | 1350 | 1365 | 23755 | 23770 | GCACTCGAGTGAACAC | 0 | 437 |
| 915845 | 1407 | 1422 | 25170 | 25185 | AGGCCTGTTGGCTGCT | 0 | 438 |
| 915865 | 1480 | 1495 | 25243 | 25258 | TTGGTCTCTGCTGGAC | 21 | 439 |
| 915885 | 1546 | 1561 | 25309 | 25324 | GGTACTTTATTGCCCA | 62 | 440 |
| 915905 | 1609 | 1624 | 25372 | 25387 | GTGACTCACAGACTCT | 81 | 441 |
| 915925 | 1635 | 1650 | 25398 | 25413 | AGAATCTGCTAGACTC | 74 | 442 |
| 915945 | 1688 | 1703 | 25451 | 25466 | CACAGCAATGCGGAGG | 56 | 443 |
| 915965 | 1770 | 1785 | 25533 | 25548 | GCGAAAGGTTGCTTCC | 66 | 444 |
| 915985 | 1791 | 1806 | 25554 | 25569 | AAGTGCTGGACCGCTG | 71 | 445 |
| 916005 | 1817 | 1832 | 25580 | 25595 | TTAACGCATGCTGATG | 69 | 446 |
| 916025 | 1850 | 1865 | 25613 | 25628 | GGGCTTCCTGGTGTCA | 58 | 447 |
| 916045 | 1886 | 1901 | 25649 | 25664 | GGGCCACGAAACAGTC | 9 | 448 |
| 916065 | 1915 | 1930 | 25678 | 25693 | CTCATGCTGGAACAGT | 86 | 449 |
| 916085 | 1959 | 1974 | 25722 | 25737 | ATCACAAGGCCCCCAC | 82 | 450 |
| 916105 | 1979 | 1994 | 25742 | 25757 | CATGGGCCAGCCTACC | 0 | 451 |
| 916125 | 2054 | 2069 | 25817 | 25832 | ACGAACTGCACCCCTT | 84 | 452 |
| 916144 | 2102 | 2117 | 25865 | 25880 | AGGTTATCATCTTTGC | 90 | 453 |
| 916164 | 2141 | 2156 | 25904 | 25919 | ATCTTGTTACCCCCGC | 88 | 454 |
| 916184 | 2266 | 2281 | 26029 | 26044 | CATCTCACTGATTCAC | 91 | 455 |
| 916204 | 2626 | 2641 | 26389 | 26404 | TGCCCTGCACACTAGA | 47 | 456 |
| 916224 | 2680 | 2695 | 26443 | 26458 | GAGGAGGCGGAAGCTC | 0 | 457 |
| 916244 | 2710 | 2725 | 26473 | 26488 | AGCCAGGTTCAAGTTG | 71 | 458 |
| 916284 | N/A | N/A | 4227 | 4242 | TCAAATGTACGGAATC | 40 | 459 |
| 916304 | N/A | N/A | 4865 | 4880 | GTACTTTAGGCTCCTG | 89 | 460 |
| 916324 | N/A | N/A | 5429 | 5444 | ACATATCAGCATTAGA | 87 | 461 |
| 916344 | N/A | N/A | 5804 | 5819 | GTCTACTATGGGAGCC | 90 | 462 |
| 916364 | N/A | N/A | 6966 | 6981 | GAAGATGCATAGAGGA | 0 | 463 |
| 916384 | N/A | N/A | 7550 | 7565 | TCACACTGGGTCACCA | 43 | 464 |
| 916544 | N/A | N/A | 12135 | 12150 | GGCAATCAGGGAGGCA | 32 | 465 |
| 916564 | N/A | N/A | 12320 | 12335 | TGACTATATAACCACA | 92 | 466 |
| 916584 | N/A | N/A | 12951 | 12966 | CCCAATTGCCACTAGG | 83 | 467 |
| 916604 | N/A | N/A | 13718 | 13733 | TCTTTACCAAGACCGC | 92 | 468 |
| 916624 | N/A | N/A | 14245 | 14260 | GACAAATTCATCAACC | 87 | 469 |
| 916644 | N/A | N/A | 14778 | 14793 | CTGTATCCAAAAGGCC | 0 | 470 |
| 916664 | N/A | N/A | 15597 | 15612 | ATACATAGCAGAGCCA | 44 | 471 |
| 916684 | N/A | N/A | 16352 | 16367 | CACCCTATCGCTCCCA | 43 | 472 |
| 916704 | N/A | N/A | 17267 | 17282 | AGTTATGTCTGACTCA | 72 | 473 |
| 916724 | N/A | N/A | 18254 | 18269 | AATATACCCCACAGCA | 40 | 474 |
| 916744 | N/A | N/A | 19288 | 19303 | GTGCATGTGTGGCTTG | 82 | 475 |
| 916763 | N/A | N/A | 20414 | 20429 | TGTAGTAAGCAATGCA | 85 | 476 |
| 916783 | N/A | N/A | 20724 | 20739 | CATATATTGCGGATGA | 24 | 477 |
| 916803 | N/A | N/A | 21005 | 21020 | GTTATTTTAACAGCTC | 95 | 478 |
| 916823 | N/A | N/A | 21561 | 21576 | ATAAGGACTTACACCA | 83 | 479 |
| 916843 | N/A | N/A | 22679 | 22694 | CAGCATGCAACCACCC | 8 | 480 |
| 916863 | N/A | N/A | 23550 | 23565 | TGGGATGCTAGGACAA | 72 | 481 |
| 916883 | N/A | N/A | 24340 | 24355 | GGAAGTAGCGGCATCC | 0 | 482 |

TABLE 8
Inhibition of PNPLA3 mRNA by 3-10-3 cEt gapmers targeting SEQ ID NO: 1 and 2

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | PNPLA3 | SEQ |
| Compound | Start | Stop | Start | Stop | Sequence | % | ID |
| Number | Site | Site | Site | Site | (5′ to 3′) | Inhibition | NO |
| 915346 | 25 | 40 | 2763 | 2778 | CATTCCCAGCGCGACG | 0 | 483 |
| 915366 | 52 | 67 | 2790 | 2805 | TACCCTGCCTCAGTGT | 0 | 484 |
| 915386 | 112 | 127 | 2850 | 2865 | TCGGGAATCGGCTCGG | 26 | 485 |
| 915406 | 195 | 210 | 2933 | 2948 | CGAAGGACAAGCTCCA | 69 | 486 |
| 915426 | 252 | 267 | 2990 | 3005 | GCTCGCTCAGGCAGCG | 0 | 487 |
| 915446 | 350 | 365 | N/A | N/A | CTGCTCCAGCGGGATA | 0 | 488 |
| 915466 | 389 | 404 | 5963 | 5978 | CCTGGCCTTCCGCACA | 40 | 489 |
| 915486 | 431 | 446 | 6005 | 6020 | GCTTAAGTTGAAGGAT | 88 | 490 |
| 915506 | 455 | 470 | 6029 | 6044 | GCAGAGACCCTGTCGG | 32 | 491 |
| 915526 | 493 | 508 | 6067 | 6082 | CCGGAGATGAGCTGGT | 4 | 492 |
| 915546 | 514 | 529 | 6088 | 6103 | GTAAGAGAGATGCCTA | 94 | 493 |
| 915566 | 562 | 577 | 6136 | 6151 | TTGGACCGAAAGTCAG | 56 | 494 |
| 915606 | 702 | 717 | 11920 | 11935 | TGGTTGTTTTGGCATC | 99 | 495 |
| 915626 | 757 | 772 | 11975 | 11990 | GTGGACTTGACTTTAG | 89 | 496 |
| 915646 | 830 | 845 | 12048 | 12063 | TCTCGAGAGAAGGTAG | 0 | 497 |
| 915666 | 882 | 897 | 13618 | 13633 | ATCCTCGAAGGCATAT | 0 | 498 |
| 915686 | 956 | 971 | 16076 | 16091 | TGAGGATGACTTCAGG | 10 | 499 |
| 915706 | 989 | 1004 | 16109 | 16124 | GCTGGGCATGGCGACC | 10 | 500 |
| 915726 | 1064 | 1079 | 16184 | 16199 | TAGCAGCTCATCTCCC | 67 | 501 |
| 915746 | 1135 | 1150 | 16255 | 16270 | GTAGCGAGCCTGGGCG | 0 | 502 |
| 915766 | 1196 | 1211 | 19036 | 19051 | TAGCAAGTTGCAAATC | 78 | 503 |
| 915786 | 1232 | 1247 | 19072 | 19087 | ACAGGGCAGCATTACA | 87 | 504 |
| 915806 | 1302 | 1317 | 23707 | 23722 | CGTCGGGCATATCTGG | 53 | 505 |
| 915826 | 1352 | 1367 | 23757 | 23772 | CAGCACTCGAGTGAAC | 24 | 506 |
| 915846 | 1408 | 1423 | 25171 | 25186 | GAGGCCTGTTGGCTGC | 0 | 507 |
| 915866 | 1508 | 1523 | 25271 | 25286 | GAGGATGGACCGCGGG | 0 | 508 |
| 915886 | 1549 | 1564 | 25312 | 25327 | GCAGGTACTTTATTGC | 0 | 509 |
| 915906 | 1610 | 1625 | 25373 | 25388 | AGTGACTCACAGACTC | 35 | 510 |
| 915926 | 1636 | 1651 | 25399 | 25414 | AAGAATCTGCTAGACT | 69 | 511 |
| 915946 | 1689 | 1704 | 25452 | 25467 | ACACAGCAATGCGGAG | 69 | 512 |
| 915966 | 1771 | 1786 | 25534 | 25549 | GGCGAAAGGTTGCTTC | 58 | 513 |
| 915986 | 1792 | 1807 | 25555 | 25570 | TAAGTGCTGGACCGCT | 70 | 514 |
| 916006 | 1818 | 1833 | 25581 | 25596 | ATTAACGCATGCTGAT | 73 | 515 |
| 916026 | 1851 | 1866 | 25614 | 25629 | TGGGCTTCCTGGTGTC | 71 | 516 |
| 916046 | 1887 | 1902 | 25650 | 25665 | AGGGCCACGAAACAGT | 61 | 517 |
| 916066 | 1917 | 1932 | 25680 | 25695 | ACCTCATGCTGGAACA | 81 | 518 |
| 916086 | 1960 | 1975 | 25723 | 25738 | CATCACAAGGCCCCCA | 48 | 519 |
| 916106 | 1980 | 1995 | 25743 | 25758 | ACATGGGCCAGCCTAC | 54 | 520 |
| 916126 | 2055 | 2070 | 25818 | 25833 | GACGAACTGCACCCCT | 77 | 521 |
| 916145 | 2105 | 2120 | 25868 | 25883 | TCAAGGTTATCATCTT | 89 | 522 |
| 916165 | 2142 | 2157 | 25905 | 25920 | CATCTTGTTACCCCCG | 89 | 523 |
| 916185 | 2270 | 2285 | 26033 | 26048 | CTAACATCTCACTGAT | 66 | 524 |
| 916205 | 2627 | 2642 | 26390 | 26405 | ATGCCCTGCACACTAG | 62 | 525 |
| 916225 | 2681 | 2696 | 26444 | 26459 | AGAGGAGGCGGAAGCT | 25 | 526 |
| 916245 | 2711 | 2726 | 26474 | 26489 | AAGCCAGGTTCAAGTT | 83 | 527 |
| 916285 | N/A | N/A | 4240 | 4255 | ATTAGGACAAGATTCA | 75 | 528 |
| 916305 | N/A | N/A | 4866 | 4881 | TGTACTTTAGGCTCCT | 93 | 529 |
| 916325 | N/A | N/A | 5430 | 5445 | AACATATCAGCATTAG | 85 | 530 |
| 916345 | N/A | N/A | 5839 | 5854 | CAAGGATGCCACCAAC | 84 | 531 |
| 916365 | N/A | N/A | 6974 | 6989 | TCATTATGGAAGATGC | 0 | 532 |
| 916385 | N/A | N/A | 7602 | 7617 | TTAACAACCCTGTCAG | 1 | 533 |
| 916545 | N/A | N/A | 12151 | 12166 | GTAACTGGTAGCTCCT | 93 | 534 |
| 916565 | N/A | N/A | 12338 | 12353 | ACCCATACTGCACCCC | 79 | 535 |
| 916585 | N/A | N/A | 12957 | 12972 | GCCTATCCCAATTGCC | 70 | 536 |
| 916605 | N/A | N/A | 13719 | 13734 | GTCTTTACCAAGACCG | 23 | 537 |
| 916625 | N/A | N/A | 14248 | 14263 | AACGACAAATTCATCA | 84 | 538 |
| 916645 | N/A | N/A | 14788 | 14803 | TGCAATCCCCCTGTAT | 17 | 539 |
| 916665 | N/A | N/A | 15598 | 15613 | AATACATAGCAGAGCC | 68 | 540 |
| 916685 | N/A | N/A | 16366 | 16381 | TGTCATGGTTGCCTCA | 70 | 541 |
| 916705 | N/A | N/A | 17273 | 17288 | ATAAGGAGTTATGTCT | 80 | 542 |
| 916725 | N/A | N/A | 18255 | 18270 | GAATATACCCCACAGC | 58 | 543 |
| 916745 | N/A | N/A | 19295 | 19310 | GTTACAGGTGCATGTG | 75 | 544 |
| 916764 | N/A | N/A | 20435 | 20450 | AGTCATCTGGAGTCAC | 69 | 545 |
| 916784 | N/A | N/A | 20756 | 20771 | TCAGACAACCCACTGA | 24 | 546 |
| 916804 | N/A | N/A | 21046 | 21061 | AGGAATCTGAATCCTA | 0 | 547 |
| 916824 | N/A | N/A | 21640 | 21655 | GATAATTTCCTAGAGC | 29 | 548 |
| 916844 | N/A | N/A | 22699 | 22714 | GAAATAAGTGCTCAGG | 73 | 549 |
| 916864 | N/A | N/A | 23582 | 23597 | CTCCAATCTGATGACT | 53 | 550 |
| 916884 | N/A | N/A | 24347 | 24362 | GAATTCAGGAAGTAGC | 50 | 551 |

TABLE 9
Inhibition of PNPLA3 mRNA by 3-10-3 cEt gapmers targeting SEQ ID NO: 1 and 2

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | PNPLA3 | SEQ |
| Compound | Start | Stop | Start | Stop | Sequence | % | ID |
| Number | Site | Site | Site | Site | (5′ to 3′) | Inhibition | NO |
| 915347 | 26 | 41 | 2764 | 2779 | GCATTCCCAGCGCGAC | 0 | 552 |
| 915367 | 58 | 73 | 2796 | 2811 | GCTCTCTACCCTGCCT | 9 | 553 |
| 915387 | 113 | 128 | 2851 | 2866 | ATCGGGAATCGGCTCG | 28 | 554 |
| 915407 | 198 | 213 | 2936 | 2951 | CCGCGAAGGACAAGCT | 0 | 555 |
| 915427 | 253 | 268 | 2991 | 3006 | TGCTCGCTCAGGCAGC | 0 | 556 |
| 915447 | 351 | 366 | N/A | N/A | TCTGCTCCAGCGGGAT | 1 | 557 |
| 915467 | 391 | 406 | 5965 | 5980 | CTCCTGGCCTTCCGCA | 29 | 558 |
| 915487 | 433 | 448 | 6007 | 6022 | TTGCTTAAGTTGAAGG | 94 | 559 |
| 915507 | 456 | 471 | 6030 | 6045 | TGCAGAGACCCTGTCG | 31 | 560 |
| 915527 | 494 | 509 | 6068 | 6083 | GCCGGAGATGAGCTGG | 0 | 561 |
| 915547 | 515 | 530 | 6089 | 6104 | GGTAAGAGAGATGCCT | 0 | 562 |
| 915567 | 563 | 578 | 6137 | 6152 | TTTGGACCGAAAGTCA | 0 | 563 |
| 915607 | 703 | 718 | 11921 | 11936 | ATGGTTGTTTTGGCAT | 35 | 564 |
| 915627 | 758 | 773 | 11976 | 11991 | CGTGGACTTGACTTTA | 85 | 565 |
| 915647 | 831 | 846 | 12049 | 12064 | CTCTCGAGAGAAGGTA | 7 | 566 |
| 915667 | 883 | 898 | 13619 | 13634 | TATCCTCGAAGGCATA | 0 | 567 |
| 915687 | 959 | 974 | 16079 | 16094 | TTCTGAGGATGACTTC | 39 | 568 |
| 915707 | 996 | 1011 | 16116 | 16131 | TTGCCCAGCTGGGCAT | 0 | 569 |
| 915727 | 1065 | 1080 | 16185 | 16200 | CTAGCAGCTCATCTCC | 58 | 570 |
| 915747 | 1136 | 1151 | 16256 | 16271 | TGTAGCGAGCCTGGGC | 16 | 571 |
| 915767 | 1197 | 1212 | 19037 | 19052 | GTAGCAAGTTGCAAAT | 80 | 572 |
| 915787 | 1233 | 1248 | 19073 | 19088 | TACAGGGCAGCATTAC | 71 | 573 |
| 915807 | 1316 | 1331 | 23721 | 23736 | CAACCACAGGACATCG | 0 | 574 |
| 915827 | 1353 | 1368 | 23758 | 23773 | TCAGCACTCGAGTGAA | 0 | 575 |
| 915847 | 1409 | 1424 | 25172 | 25187 | GGAGGCCTGTTGGCTG | 0 | 576 |
| 915867 | 1509 | 1524 | 25272 | 25287 | TGAGGATGGACCGCGG | 14 | 577 |
| 915887 | 1553 | 1568 | 25316 | 25331 | ACCAGCAGGTACTTTA | 29 | 578 |
| 915907 | 1611 | 1626 | 25374 | 25389 | AAGTGACTCACAGACT | 29 | 579 |
| 915927 | 1637 | 1652 | 25400 | 25415 | AAAGAATCTGCTAGAC | 60 | 580 |
| 915947 | 1690 | 1705 | 25453 | 25468 | TACACAGCAATGCGGA | 69 | 581 |
| 915967 | 1772 | 1787 | 25535 | 25550 | AGGCGAAAGGTTGCTT | 0 | 582 |
| 915987 | 1793 | 1808 | 25556 | 25571 | TTAAGTGCTGGACCGC | 82 | 583 |
| 916007 | 1819 | 1834 | 25582 | 25597 | AATTAACGCATGCTGA | 61 | 584 |
| 916027 | 1864 | 1879 | 25627 | 25642 | GGACCCTCTGCACTGG | 43 | 585 |
| 916047 | 1888 | 1903 | 25651 | 25666 | TAGGGCCACGAAACAG | 80 | 586 |
| 916067 | 1918 | 1933 | 25681 | 25696 | AACCTCATGCTGGAAC | 72 | 587 |
| 916087 | 1961 | 1976 | 25724 | 25739 | CCATCACAAGGCCCCC | 63 | 588 |
| 916107 | 1981 | 1996 | 25744 | 25759 | CACATGGGCCAGCCTA | 74 | 589 |
| 916127 | 2056 | 2071 | 25819 | 25834 | GGACGAACTGCACCCC | 5 | 590 |
| 916146 | 2106 | 2121 | 25869 | 25884 | GTCAAGGTTATCATCT | 88 | 591 |
| 916166 | 2143 | 2158 | 25906 | 25921 | TCATCTTGTTACCCCC | 90 | 592 |
| 916186 | 2272 | 2287 | 26035 | 26050 | TACTAACATCTCACTG | 1 | 593 |
| 916206 | 2628 | 2643 | 26391 | 26406 | AATGCCCTGCACACTA | 56 | 594 |
| 916226 | 2682 | 2697 | 26445 | 26460 | GAGAGGAGGCGGAAGC | 10 | 595 |
| 916246 | 2712 | 2727 | 26475 | 26490 | TAAGCCAGGTTCAAGT | 81 | 596 |
| 916286 | N/A | N/A | 4244 | 4259 | TTTCATTAGGACAAGA | 61 | 597 |
| 916306 | N/A | N/A | 4867 | 4882 | GTGTACTTTAGGCTCC | 97 | 598 |
| 916326 | N/A | N/A | 5431 | 5446 | GAACATATCAGCATTA | 52 | 599 |
| 916346 | N/A | N/A | 5872 | 5887 | GTAATACTTTTGGCAA | 75 | 600 |
| 916366 | N/A | N/A | 7069 | 7084 | GGTATTACAAATTATC | 10 | 601 |
| 916386 | N/A | N/A | 7603 | 7618 | CTTAACAACCCTGTCA | 0 | 602 |
| 916546 | N/A | N/A | 12152 | 12167 | AGTAACTGGTAGCTCC | 88 | 603 |
| 916566 | N/A | N/A | 12343 | 12358 | CTAATACCCATACTGC | 84 | 604 |
| 916586 | N/A | N/A | 12966 | 12981 | AACTTTGCAGCCTATC | 85 | 605 |
| 916606 | N/A | N/A | 13739 | 13754 | AGAACTAAGGCAAATC | 85 | 606 |
| 916626 | N/A | N/A | 14257 | 14272 | GTCTTGGCCAACGACA | 0 | 607 |
| 916646 | N/A | N/A | 14793 | 14808 | CAGGATGCAATCCCCC | 45 | 608 |
| 916666 | N/A | N/A | 15601 | 15616 | GCCAATACATAGCAGA | 75 | 609 |
| 916686 | N/A | N/A | 16630 | 16645 | GTCCATGAAATCCAGG | 0 | 610 |
| 916706 | N/A | N/A | 17293 | 17308 | TCTCTTAGGGCACCTC | 87 | 611 |
| 916726 | N/A | N/A | 18256 | 18271 | TGAATATACCCCACAG | 24 | 612 |
| 916746 | N/A | N/A | 19337 | 19352 | AGCTCTAGGAGTCCCC | 63 | 613 |
| 916765 | N/A | N/A | 20513 | 20528 | CCAGATTGAGTCTCCT | 91 | 614 |
| 916785 | N/A | N/A | 20775 | 20790 | AATCAAGTGCCCTCCA | 73 | 615 |
| 916805 | N/A | N/A | 21211 | 21226 | TGTAGCTGTGTGGTGG | 85 | 616 |
| 916825 | N/A | N/A | 21760 | 21775 | TACCATGATCAGGTCA | 0 | 617 |
| 916845 | N/A | N/A | 22713 | 22728 | GTAAAGATGTGAGTGA | 85 | 618 |
| 916865 | N/A | N/A | 23606 | 23621 | GTTTACAAAAGCTGCC | 17 | 619 |
| 916885 | N/A | N/A | 24375 | 24390 | TGAACTCCGGCTCAGT | 0 | 620 |

TABLE 10
Inhibition of PNPLA3 mRNA by 3-10-3 cEt gapmers targeting SEQ ID NO: 1 and 2

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | PNPLA3 | SEQ |
| Compound | Start | Stop | Start | Stop | Sequence | % | ID |
| Number | Site | Site | Site | Site | (5′ to 3′) | Inhibition | NO |
| 915348 | 28 | 43 | 2766 | 2781 | GGGCATTCCCAGCGCG | 0 | 621 |
| 915368 | 59 | 74 | 2797 | 2812 | CGCTCTCTACCCTGCC | 0 | 622 |
| 915388 | 114 | 129 | 2852 | 2867 | GATCGGGAATCGGCTC | 32 | 623 |
| 915408 | 199 | 214 | 2937 | 2952 | CCCGCGAAGGACAAGC | 6 | 624 |
| 915428 | 275 | 290 | 3013 | 3028 | GTCGCGGAGGAGGTGC | 0 | 625 |
| 915448 | 352 | 367 | N/A | N/A | GTCTGCTCCAGCGGGA | 4 | 626 |
| 915468 | 392 | 407 | 5966 | 5981 | ACTCCTGGCCTTCCGC | 87 | 627 |
| 915488 | 434 | 449 | 6008 | 6023 | CTTGCTTAAGTTGAAG | 0 | 628 |
| 915508 | 457 | 472 | 6031 | 6046 | TTGCAGAGACCCTGTC | 63 | 629 |
| 915528 | 495 | 510 | 6069 | 6084 | TGCCGGAGATGAGCTG | 0 | 630 |
| 915548 | 516 | 531 | 6090 | 6105 | TGGTAAGAGAGATGCC | 18 | 631 |
| 915568 | 564 | 579 | 6138 | 6153 | CTTTGGACCGAAAGTC | 10 | 632 |
| 915608 | 704 | 719 | 11922 | 11937 | GATGGTTGTTTTGGCA | 98 | 633 |
| 915628 | 772 | 787 | 11990 | 12005 | ACATGAAGAAAGTTCG | 41 | 634 |
| 915648 | 832 | 847 | 12050 | 12065 | GCTCTCGAGAGAAGGT | 55 | 635 |
| 915668 | 884 | 899 | 13620 | 13635 | ATATCCTCGAAGGCAT | 33 | 636 |
| 915688 | 962 | 977 | 16082 | 16097 | CCCTTCTGAGGATGAC | 11 | 637 |
| 915708 | 998 | 1013 | 16118 | 16133 | GTTTGCCCAGCTGGGC | 0 | 638 |
| 915728 | 1067 | 1082 | 16187 | 16202 | GTCTAGCAGCTCATCT | 68 | 639 |
| 915748 | 1137 | 1152 | 16257 | 16272 | CTGTAGCGAGCCTGGG | 0 | 640 |
| 915768 | 1198 | 1213 | 19038 | 19053 | GGTAGCAAGTTGCAAA | 90 | 641 |
| 915788 | 1234 | 1249 | 19074 | 19089 | GTACAGGGCAGCATTA | 69 | 642 |
| 915808 | 1317 | 1332 | 23722 | 23737 | GCAACCACAGGACATC | 51 | 643 |
| 915828 | 1354 | 1369 | 23759 | 23774 | ATCAGCACTCGAGTGA | 0 | 644 |
| 915848 | 1410 | 1425 | 25173 | 25188 | GGGAGGCCTGTTGGCT | 17 | 645 |
| 915868 | 1510 | 1525 | 25273 | 25288 | CTGAGGATGGACCGCG | 53 | 646 |
| 915888 | 1554 | 1569 | 25317 | 25332 | CACCAGCAGGTACTTT | 0 | 647 |
| 915908 | 1612 | 1627 | 25375 | 25390 | CAAGTGACTCACAGAC | 91 | 648 |
| 915928 | 1639 | 1654 | 25402 | 25417 | TGAAAGAATCTGCTAG | 59 | 649 |
| 915948 | 1691 | 1706 | 25454 | 25469 | CTACACAGCAATGCGG | 20 | 650 |
| 915968 | 1773 | 1788 | 25536 | 25551 | CAGGCGAAAGGTTGCT | 60 | 651 |
| 915988 | 1794 | 1809 | 25557 | 25572 | GTTAAGTGCTGGACCG | 86 | 652 |
| 916008 | 1820 | 1835 | 25583 | 25598 | GAATTAACGCATGCTG | 88 | 653 |
| 916028 | 1865 | 1880 | 25628 | 25643 | GGGACCCTCTGCACTG | 0 | 654 |
| 916048 | 1889 | 1904 | 25652 | 25667 | ATAGGGCCACGAAACA | 75 | 655 |
| 916068 | 1919 | 1934 | 25682 | 25697 | GAACCTCATGCTGGAA | 72 | 656 |
| 916088 | 1962 | 1977 | 25725 | 25740 | CCCATCACAAGGCCCC | 37 | 657 |
| 916108 | 1984 | 1999 | 25747 | 25762 | TCACACATGGGCCAGC | 84 | 658 |
| 916128 | 2079 | 2094 | 25842 | 25857 | CTGACAGGCAGTGTCG | 10 | 659 |
| 916147 | 2107 | 2122 | 25870 | 25885 | AGTCAAGGTTATCATC | 81 | 660 |
| 916167 | 2144 | 2159 | 25907 | 25922 | ATCATCTTGTTACCCC | 88 | 661 |
| 916187 | 2276 | 2291 | 26039 | 26054 | ATTCTACTAACATCTC | 90 | 662 |
| 916207 | 2629 | 2644 | 26392 | 26407 | GAATGCCCTGCACACT | 72 | 663 |
| 916227 | 2691 | 2706 | 26454 | 26469 | GCTCCAGTGGAGAGGA | 14 | 664 |
| 916247 | 2713 | 2728 | 26476 | 26491 | ATAAGCCAGGTTCAAG | 88 | 665 |
| 916287 | N/A | N/A | 4308 | 4323 | GTGAGAAACAAACCCT | 92 | 666 |
| 916307 | N/A | N/A | 4882 | 4897 | TCTATACCAGAGTGAG | 84 | 667 |
| 916327 | N/A | N/A | 5514 | 5529 | AGGAATGAGTCTCCCA | 17 | 668 |
| 916347 | N/A | N/A | 5873 | 5888 | GGTAATACTTTTGGCA | 70 | 669 |
| 916367 | N/A | N/A | 7106 | 7121 | CGCTTATGAAAGCATC | 0 | 670 |
| 916387 | N/A | N/A | 7605 | 7620 | CCCTTAACAACCCTGT | 28 | 671 |
| 916547 | N/A | N/A | 12167 | 12182 | TTTGATTGTGCAGACA | 98 | 672 |
| 916567 | N/A | N/A | 12345 | 12360 | TCCTAATACCCATACT | 74 | 673 |
| 916587 | N/A | N/A | 12969 | 12984 | ACAAACTTTGCAGCCT | 95 | 674 |
| 916607 | N/A | N/A | 13742 | 13757 | GTTAGAACTAAGGCAA | 94 | 675 |
| 916627 | N/A | N/A | 14301 | 14316 | GAGCAGATAAATACAC | 91 | 676 |
| 916647 | N/A | N/A | 14892 | 14907 | TGGTATCTCGCTTCCT | 0 | 677 |
| 916667 | N/A | N/A | 15613 | 15628 | TAAAGCCACGCAGCCA | 46 | 678 |
| 916687 | N/A | N/A | 16656 | 16671 | CCAGATGCAGGACCCC | 0 | 679 |
| 916707 | N/A | N/A | 17326 | 17341 | AAACTAATGCACCTGG | 43 | 680 |
| 916727 | N/A | N/A | 18257 | 18272 | CTGAATATACCCCACA | 75 | 681 |
| 916747 | N/A | N/A | 19360 | 19375 | AGCTGCTATGTGAGGC | 12 | 682 |
| 916766 | N/A | N/A | 20520 | 20535 | TCAGTAACCAGATTGA | 25 | 683 |
| 916786 | N/A | N/A | 20778 | 20793 | TTTAATCAAGTGCCCT | 81 | 684 |
| 916806 | N/A | N/A | 21216 | 21231 | CAGGATGTAGCTGTGT | 84 | 685 |
| 916826 | N/A | N/A | 21887 | 21902 | TAAGATCCCATCTTAC | 13 | 686 |
| 916846 | N/A | N/A | 22739 | 22754 | AAAGTAAACACCCACC | 42 | 687 |
| 916866 | N/A | N/A | 23625 | 23640 | GCTTACAACACTACCC | 57 | 688 |
| 916886 | N/A | N/A | 24393 | 24408 | GTAATGGGAGCCAGGC | 38 | 689 |

TABLE 11
Inhibition of PNPLA3 mRNA by 3-10-3 cEt gapmers targeting SEQ ID NO: 1 and 2

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | PNPLA3 | SEQ |
| Compound | Start | Stop | Start | Stop | Sequence | % | ID |
| Number | Site | Site | Site | Site | (5′ to 3′) | Inhibition | NO |
| 915349 | 29 | 44 | 2767 | 2782 | AGGGCATTCCCAGCGC | 0 | 690 |
| 915369 | 60 | 75 | 2798 | 2813 | GCGCTCTCTACCCTGC | 23 | 691 |
| 915389 | 115 | 130 | 2853 | 2868 | GGATCGGGAATCGGCT | 54 | 692 |
| 915409 | 200 | 215 | 2938 | 2953 | GCCCGCGAAGGACAAG | 32 | 693 |
| 915429 | 276 | 291 | 3014 | 3029 | CGTCGCGGAGGAGGTG | 0 | 694 |
| 915449 | 364 | 379 | 5938 | 5953 | AGGACCTGCAGAGTCT | 21 | 695 |
| 915469 | 394 | 409 | 5968 | 5983 | CGACTCCTGGCCTTCC | 59 | 696 |
| 915489 | 435 | 450 | 6009 | 6024 | ACTTGCTTAAGTTGAA | 86 | 697 |
| 915509 | 466 | 481 | 6040 | 6055 | GGGAGGCATTTGCAGA | 57 | 698 |
| 915529 | 496 | 511 | 6070 | 6085 | TTGCCGGAGATGAGCT | 40 | 699 |
| 915549 | 518 | 533 | 6092 | 6107 | TCTGGTAAGAGAGATG | 61 | 700 |
| 915569 | 565 | 580 | 6139 | 6154 | TCTTTGGACCGAAAGT | 9 | 701 |
| 915609 | 705 | 720 | 11923 | 11938 | TGATGGTTGTTTTGGC | 99 | 702 |
| 915629 | 776 | 791 | 11994 | 12009 | GTCCACATGAAGAAAG | 32 | 703 |
| 915649 | 833 | 848 | 12051 | 12066 | AGCTCTCGAGAGAAGG | 36 | 704 |
| 915669 | 885 | 900 | 13621 | 13636 | AATATCCTCGAAGGCA | 55 | 705 |
| 915689 | 969 | 984 | 16089 | 16104 | GATCCATCCCTTCTGA | 20 | 706 |
| 915709 | 999 | 1014 | 16119 | 16134 | TGTTTGCCCAGCTGGG | 5 | 707 |
| 915729 | 1077 | 1092 | 16197 | 16212 | GACGCAGGTGGTCTAG | 0 | 708 |
| 915749 | 1138 | 1153 | N/A | N/A | GCTGTAGCGAGCCTGG | 71 | 709 |
| 915769 | 1200 | 1215 | 19040 | 19055 | TGGGTAGCAAGTTGCA | 81 | 710 |
| 915789 | 1235 | 1250 | 19075 | 19090 | GGTACAGGGCAGCATT | 88 | 711 |
| 915809 | 1318 | 1333 | 23723 | 23738 | TGCAACCACAGGACAT | 40 | 712 |
| 915829 | 1355 | 1370 | 23760 | 23775 | CATCAGCACTCGAGTG | 0 | 713 |
| 915849 | 1424 | 1439 | 25187 | 25202 | CTCAGGTGTGCATGGG | 61 | 714 |
| 915869 | 1511 | 1526 | 25274 | 25289 | CCTGAGGATGGACCGC | 70 | 715 |
| 915889 | 1556 | 1571 | 25319 | 25334 | AGCACCAGCAGGTACT | 35 | 716 |
| 915909 | 1613 | 1628 | 25376 | 25391 | TCAAGTGACTCACAGA | 84 | 717 |
| 915929 | 1645 | 1660 | 25408 | 25423 | CACCTCTGAAAGAATC | 89 | 718 |
| 915949 | 1692 | 1707 | 25455 | 25470 | ACTACACAGCAATGCG | 33 | 719 |
| 915969 | 1774 | 1789 | 25537 | 25552 | ACAGGCGAAAGGTTGC | 88 | 720 |
| 915989 | 1795 | 1810 | 25558 | 25573 | AGTTAAGTGCTGGACC | 84 | 721 |
| 916009 | 1823 | 1838 | 25586 | 25601 | GCTGAATTAACGCATG | 67 | 722 |
| 916029 | 1866 | 1881 | 25629 | 25644 | AGGGACCCTCTGCACT | 15 | 723 |
| 916049 | 1890 | 1905 | 25653 | 25668 | AATAGGGCCACGAAAC | 52 | 724 |
| 916069 | 1920 | 1935 | 25683 | 25698 | AGAACCTCATGCTGGA | 85 | 725 |
| 916089 | 1963 | 1978 | 25726 | 25741 | CCCCATCACAAGGCCC | 20 | 726 |
| 916109 | 1985 | 2000 | 25748 | 25763 | ATCACACATGGGCCAG | 72 | 727 |
| 916129 | 2080 | 2095 | 25843 | 25858 | CCTGACAGGCAGTGTC | 15 | 728 |
| 916148 | 2108 | 2123 | 25871 | 25886 | TAGTCAAGGTTATCAT | 87 | 729 |
| 916168 | 2146 | 2161 | 25909 | 25924 | TTATCATCTTGTTACC | 82 | 730 |
| 916188 | 2279 | 2294 | 26042 | 26057 | CTTATTCTACTAACAT | 87 | 731 |
| 916208 | 2630 | 2645 | 26393 | 26408 | TGAATGCCCTGCACAC | 68 | 732 |
| 916228 | 2692 | 2707 | 26455 | 26470 | TGCTCCAGTGGAGAGG | 80 | 733 |
| 916248 | 2726 | 2741 | 26489 | 26504 | GTCCCTGCAGAAAATA | 0 | 734 |
| 916288 | N/A | N/A | 4337 | 4352 | AGCATACCACACCCCA | 75 | 735 |
| 916308 | N/A | N/A | 5086 | 5101 | GGACATGCTCAGCAGC | 68 | 736 |
| 916328 | N/A | N/A | 5533 | 5548 | TGCTGTAGGCCTCAGC | 0 | 737 |
| 916348 | N/A | N/A | 5874 | 5889 | TGGTAATACTTTTGGC | 86 | 738 |
| 916368 | N/A | N/A | 7132 | 7147 | GTAAATGGAGTCCTTC | 80 | 739 |
| 916388 | N/A | N/A | 7612 | 7627 | CATAATCCCCTTAACA | 32 | 740 |
| 916548 | N/A | N/A | 12195 | 12210 | TTAACCATCAAGGACA | 77 | 741 |
| 916568 | N/A | N/A | 12665 | 12680 | TCTTAGTGGCTGGGTA | 85 | 742 |
| 916588 | N/A | N/A | 12973 | 12988 | CCTAACAAACTTTGCA | 32 | 743 |
| 916608 | N/A | N/A | 13749 | 13764 | ACTAAGTGTTAGAACT | 76 | 744 |
| 916628 | N/A | N/A | 14338 | 14353 | CTGCAGTATCCCTAGC | 0 | 745 |
| 916648 | N/A | N/A | 15012 | 15027 | TCCCATCGGTCATTTC | 45 | 746 |
| 916668 | N/A | N/A | 15682 | 15697 | GAAACCACTATCATCA | 62 | 747 |
| 916688 | N/A | N/A | 16671 | 16686 | GTAATAGGCCAAGTCC | 0 | 748 |
| 916708 | N/A | N/A | 17327 | 17342 | CAAACTAATGCACCTG | 66 | 749 |
| 916728 | N/A | N/A | 18332 | 18347 | CCAATATCATAGCTGA | 85 | 750 |
| 916748 | N/A | N/A | 19376 | 19391 | CACAAGAGACTGGACC | 64 | 751 |
| 916767 | N/A | N/A | 20551 | 20566 | TACTATGGGATGAGTA | 0 | 752 |
| 916787 | N/A | N/A | 20779 | 20794 | TTTTAATCAAGTGCCC | 38 | 753 |
| 916807 | N/A | N/A | 21218 | 21233 | GGCAGGATGTAGCTGT | 63 | 754 |
| 916827 | N/A | N/A | 21947 | 21962 | AGTCAAACATCTTCCT | 50 | 755 |
| 916847 | N/A | N/A | 22759 | 22774 | CAGACTAACTTACTAA | 77 | 756 |
| 916867 | N/A | N/A | 23626 | 23641 | AGCTTACAACACTACC | 13 | 757 |
| 916887 | N/A | N/A | 24505 | 24520 | ATGCTACGGGCTCTCA | 0 | 758 |

TABLE 12
Inhibition of PNPLA3 mRNA by 3-10-3 cEt gapmers targeting SEQ ID NO: 1 and 2

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | PNPLA3 | SEQ |
| Compound | Start | Stop | Start | Stop | Sequence | % | ID |
| Number | Site | Site | Site | Site | (5′ to 3′) | Inhibition | NO |
| 915350 | 30 | 45 | 2768 | 2783 | CAGGGCATTCCCAGCG | 0 | 759 |
| 915370 | 82 | 97 | 2820 | 2835 | CAGCTCCGCCCGGCGC | 14 | 760 |
| 915390 | 130 | 145 | 2868 | 2883 | TTAGGATCTGGGTCGG | 88 | 761 |
| 915410 | 201 | 216 | 2939 | 2954 | AGCCCGCGAAGGACAA | 0 | 762 |
| 915430 | 295 | 310 | 3033 | 3048 | GCGCCGAACAACATGC | 0 | 763 |
| 915450 | 366 | 381 | 5940 | 5955 | AGAGGACCTGCAGAGT | 83 | 764 |
| 915470 | 395 | 410 | 5969 | 5984 | CCGACTCCTGGCCTTC | 68 | 765 |
| 915490 | 436 | 451 | 6010 | 6025 | AACTTGCTTAAGTTGA | 41 | 766 |
| 915510 | 467 | 482 | 6041 | 6056 | CGGGAGGCATTTGCAG | 44 | 767 |
| 915530 | 497 | 512 | 6071 | 6086 | TTTGCCGGAGATGAGC | 92 | 768 |
| 915550 | 519 | 534 | 6093 | 6108 | CTCTGGTAAGAGAGAT | 20 | 769 |
| 915570 | 566 | 581 | 6140 | 6155 | GTCTTTGGACCGAAAG | 19 | 770 |
| 915590 | 627 | 642 | 7857 | 7872 | GAGGGATAAGGCCACT | 83 | 771 |
| 915610 | 706 | 721 | 11924 | 11939 | GTGATGGTTGTTTTGG | 97 | 772 |
| 915630 | 782 | 797 | 12000 | 12015 | GGTGATGTCCACATGA | 87 | 773 |
| 915650 | 834 | 849 | 12052 | 12067 | AAGCTCTCGAGAGAAG | 44 | 774 |
| 915670 | 887 | 902 | 13623 | 13638 | CAAATATCCTCGAAGG | 0 | 775 |
| 915690 | 970 | 985 | 16090 | 16105 | GGATCCATCCCTTCTG | 0 | 776 |
| 915710 | 1003 | 1018 | 16123 | 16138 | CTCATGTTTGCCCAGC | 68 | 777 |
| 915730 | 1078 | 1093 | 16198 | 16213 | AGACGCAGGTGGTCTA | 0 | 778 |
| 915750 | 1139 | 1154 | N/A | N/A | TGCTGTAGCGAGCCTG | 56 | 779 |
| 915770 | 1201 | 1216 | 19041 | 19056 | ATGGGTAGCAAGTTGC | 79 | 780 |
| 915790 | 1247 | 1262 | 19087 | 19102 | TTCCACAGGCAGGGTA | 48 | 781 |
| 915810 | 1320 | 1335 | 23725 | 23740 | ACTGCAACCACAGGAC | 22 | 782 |
| 915830 | 1356 | 1371 | 23761 | 23776 | ACATCAGCACTCGAGT | 0 | 783 |
| 915850 | 1427 | 1442 | 25190 | 25205 | CTGCTCAGGTGTGCAT | 10 | 784 |
| 915870 | 1512 | 1527 | 25275 | 25290 | ACCTGAGGATGGACCG | 69 | 785 |
| 915890 | 1557 | 1572 | 25320 | 25335 | CAGCACCAGCAGGTAC | 62 | 786 |
| 915910 | 1617 | 1632 | 25380 | 25395 | CTCCTCAAGTGACTCA | 83 | 787 |
| 915930 | 1648 | 1663 | 25411 | 25426 | TAGCACCTCTGAAAGA | 55 | 788 |
| 915950 | 1693 | 1708 | 25456 | 25471 | CACTACACAGCAATGC | 74 | 789 |
| 915970 | 1775 | 1790 | 25538 | 25553 | CACAGGCGAAAGGTTG | 72 | 790 |
| 915990 | 1797 | 1812 | 25560 | 25575 | AGAGTTAAGTGCTGGA | 92 | 791 |
| 916010 | 1824 | 1839 | 25587 | 25602 | AGCTGAATTAACGCAT | 0 | 792 |
| 916030 | 1867 | 1882 | 25630 | 25645 | AAGGGACCCTCTGCAC | 38 | 793 |
| 916050 | 1891 | 1906 | 25654 | 25669 | TAATAGGGCCACGAAA | 53 | 794 |
| 916070 | 1921 | 1936 | 25684 | 25699 | AAGAACCTCATGCTGG | 24 | 795 |
| 916090 | 1964 | 1979 | 25727 | 25742 | CCCCCATCACAAGGCC | 24 | 796 |
| 916110 | 1986 | 2001 | 25749 | 25764 | GATCACACATGGGCCA | 0 | 797 |
| 916130 | 2081 | 2096 | 25844 | 25859 | ACCTGACAGGCAGTGT | 54 | 798 |
| 916149 | 2109 | 2124 | 25872 | 25887 | GTAGTCAAGGTTATCA | 87 | 799 |
| 916169 | 2154 | 2169 | 25917 | 25932 | TAAGTAGATTATCATC | 79 | 800 |
| 916189 | 2282 | 2297 | 26045 | 26060 | AGGCTTATTCTACTAA | 85 | 801 |
| 916209 | 2631 | 2646 | 26394 | 26409 | GTGAATGCCCTGCACA | 59 | 802 |
| 916229 | 2693 | 2708 | 26456 | 26471 | GTGCTCCAGTGGAGAG | 54 | 803 |
| 916249 | 2727 | 2742 | 26490 | 26505 | GGTCCCTGCAGAAAAT | 38 | 804 |
| 916289 | N/A | N/A | 4338 | 4353 | AAGCATACCACACCCC | 79 | 805 |
| 916309 | N/A | N/A | 5278 | 5293 | AATCTTGGGATGCACA | 95 | 806 |
| 916329 | N/A | N/A | 5569 | 5584 | CATCATGGCTTCCAGT | 79 | 807 |
| 916349 | N/A | N/A | 5879 | 5894 | TGGGATGGTAATACTT | 0 | 808 |
| 916369 | N/A | N/A | 7134 | 7149 | AAGTAAATGGAGTCCT | 5 | 809 |
| 916389 | N/A | N/A | 7615 | 7630 | TTGCATAATCCCCTTA | 33 | 810 |
| 916409 | N/A | N/A | 8165 | 8180 | TTAACTAGATCACTGA | 58 | 811 |
| 916429 | N/A | N/A | 9109 | 9124 | TCCTAATGCGAGTCCC | 86 | 812 |
| 916449 | N/A | N/A | 9522 | 9537 | TGCTGCTGGGTGCACT | 45 | 813 |
| 916469 | N/A | N/A | 10199 | 10214 | GGTGATGACACAGCAT | 94 | 814 |
| 916489 | N/A | N/A | 10382 | 10397 | GCCATGTACAACTTTT | 52 | 815 |
| 916509 | N/A | N/A | 11152 | 11167 | TACAATTTGGACAGAG | 71 | 816 |
| 916529 | N/A | N/A | 11546 | 11561 | ACCTATAGGAGTGCCC | 35 | 817 |
| 916549 | N/A | N/A | 12204 | 12219 | TTATTTCCGTTAACCA | 97 | 818 |
| 916569 | N/A | N/A | 12672 | 12687 | AGAATCATCTTAGTGG | 94 | 819 |
| 916589 | N/A | N/A | 12989 | 13004 | CGGAATAAGCCTCCAC | 0 | 820 |
| 916609 | N/A | N/A | 13752 | 13767 | GGCACTAAGTGTTAGA | 57 | 821 |
| 916629 | N/A | N/A | 14375 | 14390 | TCTCACAAGGCTGGCA | 84 | 822 |
| 916649 | N/A | N/A | 15137 | 15152 | GCCATACCGGCTCCCT | 30 | 823 |
| 916669 | N/A | N/A | 15691 | 15706 | GGCCTTACAGAAACCA | 15 | 824 |
| 916689 | N/A | N/A | 16672 | 16687 | AGTAATAGGCCAAGTC | 16 | 825 |
| 916709 | N/A | N/A | 17328 | 17343 | ACAAACTAATGCACCT | 42 | 826 |
| 916729 | N/A | N/A | 18333 | 18348 | TCCAATATCATAGCTG | 32 | 827 |
| 916749 | N/A | N/A | 19445 | 19460 | CTACTAGGCATCTCTA | 32 | 828 |
| 916768 | N/A | N/A | 20553 | 20568 | CTTACTATGGGATGAG | 83 | 829 |
| 916788 | N/A | N/A | 20808 | 20823 | TAATATTCAGACCAGG | 94 | 830 |
| 916808 | N/A | N/A | 21252 | 21267 | CCATGCATGGCACAGT | 4 | 831 |
| 916828 | N/A | N/A | 21968 | 21983 | AGACAGGAATCCAACC | 0 | 832 |
| 916848 | N/A | N/A | 22767 | 22782 | GGACATGACAGACTAA | 96 | 833 |
| 916868 | N/A | N/A | 23637 | 23652 | GCAGACACAACAGCTT | 40 | 834 |
| 916888 | N/A | N/A | 24507 | 24522 | CCATGCTACGGGCTCT | 0 | 835 |

TABLE 13
Inhibition of PNPLA3 mRNA by 3-10-3 cEt gapmers targeting SEQ ID NO: 1 and 2

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | PNPLA3 | SEQ |
| Compound | Start | Stop | Start | Stop | Sequence | % | ID |
| Number | Site | Site | Site | Site | (5′ to 3′) | Inhibition | NO |
| 915351 | 33 | 48 | 2771 | 2786 | GGCCAGGGCATTCCCA | 0 | 836 |
| 915371 | 83 | 98 | 2821 | 2836 | GCAGCTCCGCCCGGCG | 2 | 837 |
| 915391 | 132 | 147 | 2870 | 2885 | GGTTAGGATCTGGGTC | 54 | 838 |
| 915411 | 222 | 237 | 2960 | 2975 | GGTAGAAGCCCAGGAA | 57 | 839 |
| 915431 | 321 | 336 | 3059 | 3074 | CGACGCAGTGCAACGC | 49 | 840 |
| 915451 | 368 | 383 | 5942 | 5957 | TGAGAGGACCTGCAGA | 0 | 841 |
| 915471 | 400 | 415 | 5974 | 5989 | ATGTTCCGACTCCTGG | 82 | 842 |
| 915491 | 437 | 452 | 6011 | 6026 | GAACTTGCTTAAGTTG | 23 | 843 |
| 915511 | 468 | 483 | 6042 | 6057 | CCGGGAGGCATTTGCA | 0 | 844 |
| 915531 | 498 | 513 | 6072 | 6087 | TTTTGCCGGAGATGAG | 84 | 845 |
| 915551 | 520 | 535 | 6094 | 6109 | ACTCTGGTAAGAGAGA | 5 | 846 |
| 915571 | 567 | 582 | 6141 | 6156 | CGTCTTTGGACCGAAA | 64 | 847 |
| 915611 | 708 | 723 | 11926 | 11941 | CGGTGATGGTTGTTTT | 98 | 848 |
| 915631 | 783 | 798 | 12001 | 12016 | TGGTGATGTCCACATG | 0 | 849 |
| 915651 | 835 | 850 | 12053 | 12068 | AAAGCTCTCGAGAGAA | 35 | 850 |
| 915671 | 890 | 905 | 13626 | 13641 | ATCCAAATATCCTCGA | 42 | 851 |
| 915691 | 971 | 986 | 16091 | 16106 | AGGATCCATCCCTTCT | 0 | 852 |
| 915711 | 1005 | 1020 | 16125 | 16140 | GACTCATGTTTGCCCA | 73 | 853 |
| 915731 | 1079 | 1094 | 16199 | 16214 | GAGACGCAGGTGGTCT | 0 | 854 |
| 915751 | 1140 | 1155 | N/A | N/A | GTGCTGTAGCGAGCCT | 0 | 855 |
| 915771 | 1202 | 1217 | 19042 | 19057 | AATGGGTAGCAAGTTG | 80 | 856 |
| 915791 | 1248 | 1263 | 19088 | 19103 | ATTCCACAGGCAGGGT | 37 | 857 |
| 915811 | 1327 | 1342 | 23732 | 23747 | GTCACCCACTGCAACC | 52 | 858 |
| 915831 | 1357 | 1372 | 23762 | 23777 | CACATCAGCACTCGAG | 29 | 859 |
| 915851 | 1429 | 1444 | 25192 | 25207 | TCCTGCTCAGGTGTGC | 2 | 860 |
| 915871 | 1513 | 1528 | 25276 | 25291 | GACCTGAGGATGGACC | 20 | 861 |
| 915891 | 1558 | 1573 | 25321 | 25336 | TCAGCACCAGCAGGTA | 68 | 862 |
| 915911 | 1620 | 1635 | 25383 | 25398 | CGCCTCCTCAAGTGAC | 48 | 863 |
| 915931 | 1652 | 1667 | 25415 | 25430 | ACTTTAGCACCTCTGA | 93 | 864 |
| 915951 | 1695 | 1710 | 25458 | 25473 | GTCACTACACAGCAAT | 84 | 865 |
| 915971 | 1776 | 1791 | 25539 | 25554 | GCACAGGCGAAAGGTT | 74 | 866 |
| 915991 | 1798 | 1813 | 25561 | 25576 | TAGAGTTAAGTGCTGG | 84 | 867 |
| 916011 | 1825 | 1840 | 25588 | 25603 | CAGCTGAATTAACGCA | 0 | 868 |
| 916031 | 1868 | 1883 | 25631 | 25646 | TAAGGGACCCTCTGCA | 54 | 869 |
| 916051 | 1892 | 1907 | 25655 | 25670 | TTAATAGGGCCACGAA | 75 | 870 |
| 916071 | 1922 | 1937 | 25685 | 25700 | TAAGAACCTCATGCTG | 56 | 871 |
| 916091 | 1965 | 1980 | 25728 | 25743 | CCCCCCATCACAAGGC | 9 | 872 |
| 916111 | 1987 | 2002 | 25750 | 25765 | AGATCACACATGGGCC | 26 | 873 |
| 916131 | 2084 | 2099 | 25847 | 25862 | ACCACCTGACAGGCAG | 80 | 874 |
| 916150 | 2110 | 2125 | 25873 | 25888 | AGTAGTCAAGGTTATC | 92 | 875 |
| 916170 | 2174 | 2189 | 25937 | 25952 | TGAAAAAGGTGTTCTA | 49 | 876 |
| 916190 | 2283 | 2298 | 26046 | 26061 | AAGGCTTATTCTACTA | 79 | 877 |
| 916210 | 2633 | 2648 | 26396 | 26411 | AGGTGAATGCCCTGCA | 71 | 878 |
| 916230 | 2694 | 2709 | 26457 | 26472 | TGTGCTCCAGTGGAGA | 75 | 879 |
| 916250 | 2728 | 2743 | 26491 | 26506 | TGGTCCCTGCAGAAAA | 79 | 880 |
| 916290 | N/A | N/A | 4397 | 4412 | TGCCTACTGGCTCACA | 14 | 881 |
| 916310 | N/A | N/A | 5279 | 5294 | AAATCTTGGGATGCAC | 94 | 882 |
| 916330 | N/A | N/A | 5572 | 5587 | TGACATCATGGCTTCC | 93 | 883 |
| 916350 | N/A | N/A | 6158 | 6173 | GCTTACATCCACGACT | 0 | 884 |
| 916370 | N/A | N/A | 7135 | 7150 | CAAGTAAATGGAGTCC | 77 | 885 |
| 916390 | N/A | N/A | 7620 | 7635 | ATCTATTGCATAATCC | 86 | 886 |
| 916550 | N/A | N/A | 12205 | 12220 | TTTATTTCCGTTAACC | 96 | 887 |
| 916570 | N/A | N/A | 12694 | 12709 | TTCTTGACCGTGTTTC | 98 | 888 |
| 916590 | N/A | N/A | 12990 | 13005 | CCGGAATAAGCCTCCA | 47 | 889 |
| 916610 | N/A | N/A | 13822 | 13837 | TGTACAATGGGACGGA | 69 | 890 |
| 916630 | N/A | N/A | 14418 | 14433 | ATCGACACAGCATCAC | 92 | 891 |
| 916650 | N/A | N/A | 15138 | 15153 | TGCCATACCGGCTCCC | 0 | 892 |
| 916670 | N/A | N/A | 15758 | 15773 | GGTTTATAACAACTGA | 89 | 893 |
| 916690 | N/A | N/A | 16722 | 16737 | GCCTTGAGGTGGGTGG | 0 | 894 |
| 916710 | N/A | N/A | 17512 | 17527 | AGTCATGGGATGTGCA | 58 | 895 |
| 916730 | N/A | N/A | 18395 | 18410 | ATGTTTGGAAGTCGCC | 92 | 896 |
| 916750 | N/A | N/A | 19473 | 19488 | AAGGATCCTGCTTCTA | 9 | 897 |
| 916769 | N/A | N/A | 20554 | 20569 | GCTTACTATGGGATGA | 62 | 898 |
| 916789 | N/A | N/A | 20809 | 20824 | GTAATATTCAGACCAG | 96 | 899 |
| 916809 | N/A | N/A | 21254 | 21269 | ATCCATGCATGGCACA | 72 | 900 |
| 916829 | N/A | N/A | 21979 | 21994 | GTCAGACACGGAGACA | 0 | 901 |
| 916849 | N/A | N/A | 23110 | 23125 | GGCTTTTGAAGGAGAG | 84 | 902 |
| 916869 | N/A | N/A | 23787 | 23802 | TATTTACCTGGAGGCG | 0 | 903 |
| 916889 | N/A | N/A | 24612 | 24627 | CAAATCGGATCTTTGC | 44 | 904 |

TABLE 14
Inhibition of PNPLA3 mRNA by 3-10-3 cEt gapmers targeting SEQ ID NO: 1 and 2

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | PNPLA3 | SEQ |
| Compound | Start | Stop | Start | Stop | Sequence | % | ID |
| Number | Site | Site | Site | Site | (5′ to 3′) | Inhibition | NO |
| 915352 | 34 | 49 | 2772 | 2787 | CGGCCAGGGCATTCCC | 0 | 905 |
| 915372 | 86 | 101 | 2824 | 2839 | GCAGCAGCTCCGCCCG | 48 | 906 |
| 915392 | 135 | 150 | 2873 | 2888 | GCGGGTTAGGATCTGG | 25 | 907 |
| 915412 | 225 | 240 | 2963 | 2978 | CGTGGTAGAAGCCCAG | 2 | 908 |
| 915432 | 322 | 337 | 3060 | 3075 | CCGACGCAGTGCAACG | 31 | 909 |
| 915452 | 371 | 386 | 5945 | 5960 | ATCTGAGAGGACCTGC | 72 | 910 |
| 915472 | 401 | 416 | 5975 | 5990 | AATGTTCCGACTCCTG | 77 | 911 |
| 915492 | 438 | 453 | 6012 | 6027 | GGAACTTGCTTAAGTT | 55 | 912 |
| 915512 | 469 | 484 | 6043 | 6058 | GCCGGGAGGCATTTGC | 3 | 913 |
| 915532 | 499 | 514 | 6073 | 6088 | ATTTTGCCGGAGATGA | 86 | 914 |
| 915552 | 521 | 536 | 6095 | 6110 | CACTCTGGTAAGAGAG | 8 | 915 |
| 915612 | 709 | 724 | 11927 | 11942 | ACGGTGATGGTTGTTT | 87 | 916 |
| 915632 | 787 | 802 | 12005 | 12020 | AGCTTGGTGATGTCCA | 38 | 917 |
| 915652 | 836 | 851 | 12054 | 12069 | AAAAGCTCTCGAGAGA | 0 | 918 |
| 915672 | 891 | 906 | 13627 | 13642 | CATCCAAATATCCTCG | 82 | 919 |
| 915692 | 972 | 987 | 16092 | 16107 | CAGGATCCATCCCTTC | 0 | 920 |
| 915712 | 1006 | 1021 | 16126 | 16141 | AGACTCATGTTTGCCC | 77 | 921 |
| 915732 | 1081 | 1096 | 16201 | 16216 | CTGAGACGCAGGTGGT | 87 | 922 |
| 915752 | 1141 | 1156 | N/A | N/A | AGTGCTGTAGCGAGCC | 1 | 923 |
| 915772 | 1203 | 1218 | 19043 | 19058 | TAATGGGTAGCAAGTT | 77 | 924 |
| 915792 | 1257 | 1272 | 19097 | 19112 | CAATGGCAGATTCCAC | 56 | 925 |
| 915812 | 1328 | 1343 | 23733 | 23748 | GGTCACCCACTGCAAC | 58 | 926 |
| 915832 | 1358 | 1373 | 23763 | 23778 | ACACATCAGCACTCGA | 66 | 927 |
| 915852 | 1430 | 1445 | 25193 | 25208 | GTCCTGCTCAGGTGTG | 52 | 928 |
| 915872 | 1518 | 1533 | 25281 | 25296 | GGCTGGACCTGAGGAT | 0 | 929 |
| 915892 | 1570 | 1585 | 25333 | 25348 | GTGGAGAGCCCCTCAG | 47 | 930 |
| 915912 | 1621 | 1636 | 25384 | 25399 | TCGCCTCCTCAAGTGA | 8 | 931 |
| 915932 | 1654 | 1669 | 25417 | 25432 | AAACTTTAGCACCTCT | 90 | 932 |
| 915952 | 1696 | 1711 | 25459 | 25474 | GGTCACTACACAGCAA | 82 | 933 |
| 915972 | 1777 | 1792 | 25540 | 25555 | TGCACAGGCGAAAGGT | 64 | 934 |
| 915992 | 1799 | 1814 | 25562 | 25577 | TTAGAGTTAAGTGCTG | 91 | 935 |
| 916012 | 1826 | 1841 | 25589 | 25604 | CCAGCTGAATTAACGC | 32 | 936 |
| 916032 | 1869 | 1884 | 25632 | 25647 | GTAAGGGACCCTCTGC | 73 | 937 |
| 916052 | 1894 | 1909 | 25657 | 25672 | CATTAATAGGGCCACG | 81 | 938 |
| 916072 | 1923 | 1938 | 25686 | 25701 | CTAAGAACCTCATGCT | 70 | 939 |
| 916092 | 1966 | 1981 | 25729 | 25744 | ACCCCCCATCACAAGG | 30 | 940 |
| 916112 | 1988 | 2003 | 25751 | 25766 | AAGATCACACATGGGC | 86 | 941 |
| 916132 | 2085 | 2100 | 25848 | 25863 | GACCACCTGACAGGCA | 61 | 942 |
| 916151 | 2111 | 2126 | 25874 | 25889 | TAGTAGTCAAGGTTAT | 88 | 943 |
| 916171 | 2176 | 2191 | 25939 | 25954 | GGTGAAAAAGGTGTTC | 84 | 944 |
| 916191 | 2284 | 2299 | 26047 | 26062 | TAAGGCTTATTCTACT | 76 | 945 |
| 916211 | 2634 | 2649 | 26397 | 26412 | GAGGTGAATGCCCTGC | 0 | 946 |
| 916231 | 2695 | 2710 | 26458 | 26473 | GTGTGCTCCAGTGGAG | 87 | 947 |
| 916251 | 2729 | 2744 | 26492 | 26507 | CTGGTCCCTGCAGAAA | 67 | 948 |
| 916291 | N/A | N/A | 4419 | 4434 | CAATGCTACTTGCCCC | 68 | 949 |
| 916311 | N/A | N/A | 5280 | 5295 | TAAATCTTGGGATGCA | 94 | 950 |
| 916331 | N/A | N/A | 5576 | 5591 | ACAATGACATCATGGC | 97 | 951 |
| 916351 | N/A | N/A | 6165 | 6180 | GCAAACTGCTTACATC | 0 | 952 |
| 916371 | N/A | N/A | 7172 | 7187 | GTTAGACGCGCCAGGC | 7 | 953 |
| 916391 | N/A | N/A | 7624 | 7639 | TCTCATCTATTGCATA | 0 | 954 |
| 916551 | N/A | N/A | 12206 | 12221 | TTTTATTTCCGTTAAC | 73 | 955 |
| 916571 | N/A | N/A | 12714 | 12729 | TAAACTACCGAACGCA | 96 | 956 |
| 916591 | N/A | N/A | 12991 | 13006 | CCCGGAATAAGCCTCC | 47 | 957 |
| 916611 | N/A | N/A | 13823 | 13838 | CTGTACAATGGGACGG | 23 | 958 |
| 916631 | N/A | N/A | 14422 | 14437 | TCCCATCGACACAGCA | 95 | 959 |
| 916651 | N/A | N/A | 15206 | 15221 | GGAATATTGCCAGGTA | 95 | 960 |
| 916671 | N/A | N/A | 15759 | 15774 | TGGTTTATAACAACTG | 29 | 961 |
| 916691 | N/A | N/A | 16746 | 16761 | ATTAGGAGAGGTCTCA | 55 | 962 |
| 916711 | N/A | N/A | 17602 | 17617 | CTTGATAGTGAATGTG | 90 | 963 |
| 916731 | N/A | N/A | 18859 | 18874 | GGCACTCACAAAAGCG | 10 | 964 |
| 916751 | N/A | N/A | 20182 | 20197 | CCCTATGTTCTACTTT | 54 | 965 |
| 916770 | N/A | N/A | 20572 | 20587 | CAACATCTCTAGCTGG | 82 | 966 |
| 916790 | N/A | N/A | 20810 | 20825 | GGTAATATTCAGACCA | 0 | 967 |
| 916810 | N/A | N/A | 21265 | 21280 | TGAAGCTACAGATCCA | 74 | 968 |
| 916830 | N/A | N/A | 22042 | 22057 | GGAAATCTGTCAGAGC | 18 | 969 |
| 916850 | N/A | N/A | 23142 | 23157 | GAATCTAGGAAGGCGA | 77 | 970 |
| 916870 | N/A | N/A | 23789 | 23804 | AGTATTTACCTGGAGG | 0 | 971 |
| 916890 | N/A | N/A | 24738 | 24753 | AGCCTTAGGAAGCCTC | 16 | 972 |

TABLE 15
Inhibition of PNPLA3 mRNA by 3-10-3 cEt gapmers targeting SEQ ID NO: 1 and 2

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | PNPLA3 | SEQ |
| Compound | Start | Stop | Start | Stop | Sequence | % | ID |
| Number | Site | Site | Site | Site | (5′ to 3′) | Inhibition | NO |
| 915353 | 35 | 50 | 2773 | 2788 | TCGGCCAGGGCATTCC | 0 | 973 |
| 915373 | 87 | 102 | 2825 | 2840 | CGCAGCAGCTCCGCCC | 0 | 974 |
| 915393 | 136 | 151 | 2874 | 2889 | CGCGGGTTAGGATCTG | 0 | 975 |
| 915413 | 239 | 254 | 2977 | 2992 | GCGGGTCGCCCCGACG | 0 | 976 |
| 915433 | 325 | 340 | 3063 | 3078 | ACGCCGACGCAGTGCA | 0 | 977 |
| 915453 | 372 | 387 | 5946 | 5961 | GATCTGAGAGGACCTG | 24 | 978 |
| 915473 | 402 | 417 | 5976 | 5991 | CAATGTTCCGACTCCT | 73 | 979 |
| 915493 | 441 | 456 | 6015 | 6030 | GGAGGAACTTGCTTAA | 87 | 980 |
| 915513 | 470 | 485 | 6044 | 6059 | GGCCGGGAGGCATTTG | 0 | 981 |
| 915533 | 500 | 515 | 6074 | 6089 | TATTTTGCCGGAGATG | 75 | 982 |
| 915553 | 522 | 537 | 6096 | 6111 | ACACTCTGGTAAGAGA | 0 | 983 |
| 915613 | 710 | 725 | 11928 | 11943 | CACGGTGATGGTTGTT | 64 | 984 |
| 915633 | 788 | 803 | 12006 | 12021 | GAGCTTGGTGATGTCC | 74 | 985 |
| 915653 | 837 | 852 | 12055 | 12070 | CAAAAGCTCTCGAGAG | 0 | 986 |
| 915673 | 892 | 907 | 13628 | 13643 | GCATCCAAATATCCTC | 81 | 987 |
| 915693 | 973 | 988 | 16093 | 16108 | TCAGGATCCATCCCTT | 10 | 988 |
| 915713 | 1007 | 1022 | 16127 | 16142 | CAGACTCATGTTTGCC | 0 | 989 |
| 915733 | 1082 | 1097 | 16202 | 16217 | GCTGAGACGCAGGTGG | 64 | 990 |
| 915753 | 1142 | 1157 | N/A | N/A | CAGTGCTGTAGCGAGC | 0 | 991 |
| 915773 | 1204 | 1219 | 19044 | 19059 | CTAATGGGTAGCAAGT | 72 | 992 |
| 915793 | 1258 | 1273 | 19098 | 19113 | GCAATGGCAGATTCCA | 57 | 993 |
| 915813 | 1329 | 1344 | 23734 | 23749 | AGGTCACCCACTGCAA | 56 | 994 |
| 915833 | 1359 | 1374 | 23764 | 23779 | GACACATCAGCACTCG | 43 | 995 |
| 915853 | 1431 | 1446 | 25194 | 25209 | AGTCCTGCTCAGGTGT | 66 | 996 |
| 915873 | 1525 | 1540 | 25288 | 25303 | AAGTTCAGGCTGGACC | 54 | 997 |
| 915893 | 1571 | 1586 | 25334 | 25349 | GGTGGAGAGCCCCTCA | 0 | 998 |
| 915913 | 1622 | 1637 | 25385 | 25400 | CTCGCCTCCTCAAGTG | 52 | 999 |
| 915933 | 1660 | 1675 | 25423 | 25438 | GATGGGAAACTTTAGC | 85 | 1000 |
| 915953 | 1697 | 1712 | 25460 | 25475 | GGGTCACTACACAGCA | 78 | 1001 |
| 915973 | 1778 | 1793 | 25541 | 25556 | CTGCACAGGCGAAAGG | 35 | 1002 |
| 915993 | 1800 | 1815 | 25563 | 25578 | ATTAGAGTTAAGTGCT | 63 | 1003 |
| 916013 | 1827 | 1842 | 25590 | 25605 | ACCAGCTGAATTAACG | 66 | 1004 |
| 916033 | 1873 | 1888 | 25636 | 25651 | GTCAGTAAGGGACCCT | 52 | 1005 |
| 916053 | 1897 | 1912 | 25660 | 25675 | GACCATTAATAGGGCC | 51 | 1006 |
| 916073 | 1924 | 1939 | 25687 | 25702 | TCTAAGAACCTCATGC | 55 | 1007 |
| 916093 | 1967 | 1982 | 25730 | 25745 | TACCCCCCATCACAAG | 15 | 1008 |
| 916113 | 1990 | 2005 | 25753 | 25768 | ACAAGATCACACATGG | 72 | 1009 |
| 916133 | 2086 | 2101 | 25849 | 25864 | AGACCACCTGACAGGC | 79 | 1010 |
| 916152 | 2112 | 2127 | 25875 | 25890 | TTAGTAGTCAAGGTTA | 84 | 1011 |
| 916172 | 2177 | 2192 | 25940 | 25955 | AGGTGAAAAAGGTGTT | 88 | 1012 |
| 916192 | 2285 | 2300 | 26048 | 26063 | TTAAGGCTTATTCTAC | 82 | 1013 |
| 916212 | 2635 | 2650 | 26398 | 26413 | TGAGGTGAATGCCCTG | 58 | 1014 |
| 916232 | 2696 | 2711 | 26459 | 26474 | TGTGTGCTCCAGTGGA | 89 | 1015 |
| 916252 | 2730 | 2745 | 26493 | 26508 | GCTGGTCCCTGCAGAA | 44 | 1016 |
| 916272 | N/A | N/A | 3328 | 3343 | GGGACGCACGAGAGTC | 0 | 1017 |
| 916292 | N/A | N/A | 4432 | 4447 | GTCAATAGCTTCACAA | 86 | 1018 |
| 916312 | N/A | N/A | 5281 | 5296 | ATAAATCTTGGGATGC | 92 | 1019 |
| 916332 | N/A | N/A | 5577 | 5592 | CACAATGACATCATGG | 95 | 1020 |
| 916352 | N/A | N/A | 6170 | 6185 | GATAAGCAAACTGCTT | 19 | 1021 |
| 916372 | N/A | N/A | 7192 | 7207 | GAGGATGCAACTGGCT | 84 | 1022 |
| 916392 | N/A | N/A | 7644 | 7659 | TCGGACTTCAGGCCCA | 0 | 1023 |
| 916552 | N/A | N/A | 12208 | 12223 | CCTTTTATTTCCGTTA | 97 | 1024 |
| 916572 | N/A | N/A | 12745 | 12760 | GCATACTAAAACCACC | 85 | 1025 |
| 916592 | N/A | N/A | 13375 | 13390 | GACTTTGCAGGCACCC | 92 | 1026 |
| 916612 | N/A | N/A | 13909 | 13924 | TGACATCCCAGTTCAA | 30 | 1027 |
| 916632 | N/A | N/A | 14427 | 14442 | TACTTTCCCATCGACA | 81 | 1028 |
| 916652 | N/A | N/A | 15207 | 15222 | AGGAATATTGCCAGGT | 88 | 1029 |
| 916672 | N/A | N/A | 15768 | 15783 | GGTTAGTGTTGGTTTA | 92 | 1030 |
| 916692 | N/A | N/A | 16790 | 16805 | CATTCGATGGAGGTTC | 58 | 1031 |
| 916712 | N/A | N/A | 17629 | 17644 | GGCGGATTTCCCCACT | 11 | 1032 |
| 916732 | N/A | N/A | 18894 | 18909 | TAAAATACGCCCGTCC | 7 | 1033 |
| 916752 | N/A | N/A | 20183 | 20198 | TCCCTATGTTCTACTT | 32 | 1034 |
| 916771 | N/A | N/A | 20574 | 20589 | ATCAACATCTCTAGCT | 46 | 1035 |
| 916791 | N/A | N/A | 20811 | 20826 | GGGTAATATTCAGACC | 43 | 1036 |
| 916811 | N/A | N/A | 21313 | 21328 | TTTACTAGAGACTCTG | 69 | 1037 |
| 916831 | N/A | N/A | 22071 | 22086 | GTAGGATAGGACTAGA | 45 | 1038 |
| 916851 | N/A | N/A | 23219 | 23234 | ATAAATGCCTGACCAC | 64 | 1039 |
| 916871 | N/A | N/A | 23861 | 23876 | TGTTTCTAGAATGTCG | 68 | 1040 |
| 916891 | N/A | N/A | 24873 | 24888 | GCCTATCAGTTTCCCC | 0 | 1041 |

TABLE 16
Inhibition of PNPLA3 mRNA by 3-10-3 cEt gapmers targeting SEQ ID NO: 1 and 2

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | PNPLA3 | SEQ |
| Compound | Start | Stop | Start | Stop | Sequence | % | ID |
| Number | Site | Site | Site | Site | (5′ to 3′) | Inhibition | NO |
| 915354 | 36 | 51 | 2774 | 2789 | CTCGGCCAGGGCATTC | 0 | 1042 |
| 915374 | 89 | 104 | 2827 | 2842 | TCCGCAGCAGCTCCGC | 60 | 1043 |
| 915394 | 137 | 152 | 2875 | 2890 | GCGCGGGTTAGGATCT | 0 | 1044 |
| 915414 | 240 | 255 | 2978 | 2993 | AGCGGGTCGCCCCGAC | 21 | 1045 |
| 915434 | 337 | 352 | 3075 | 3090 | ATACCGGAGAGGACGC | 85 | 1046 |
| 915454 | 374 | 389 | 5948 | 5963 | AAGATCTGAGAGGACC | 24 | 1047 |
| 915474 | 403 | 418 | 5977 | 5992 | CCAATGTTCCGACTCC | 95 | 1048 |
| 915494 | 442 | 457 | 6016 | 6031 | CGGAGGAACTTGCTTA | 93 | 1049 |
| 915514 | 471 | 486 | 6045 | 6060 | TGGCCGGGAGGCATTT | 0 | 1050 |
| 915534 | 501 | 516 | 6075 | 6090 | CTATTTTGCCGGAGAT | 87 | 1051 |
| 915554 | 523 | 538 | 6097 | 6112 | GACACTCTGGTAAGAG | 26 | 1052 |
| 915614 | 711 | 726 | 11929 | 11944 | ACACGGTGATGGTTGT | 46 | 1053 |
| 915634 | 791 | 806 | 12009 | 12024 | ACTGAGCTTGGTGATG | 87 | 1054 |
| 915654 | 838 | 853 | 12056 | 12071 | ACAAAAGCTCTCGAGA | 0 | 1055 |
| 915674 | 900 | 915 | 13636 | 13651 | ACCTGAATGCATCCAA | 93 | 1056 |
| 915694 | 974 | 989 | 16094 | 16109 | CTCAGGATCCATCCCT | 43 | 1057 |
| 915714 | 1008 | 1023 | 16128 | 16143 | CCAGACTCATGTTTGC | 0 | 1058 |
| 915734 | 1083 | 1098 | 16203 | 16218 | TGCTGAGACGCAGGTG | 50 | 1059 |
| 915754 | 1143 | 1158 | N/A | N/A | TCAGTGCTGTAGCGAG | 42 | 1060 |
| 915774 | 1208 | 1223 | 19048 | 19063 | TATCCTAATGGGTAGC | 53 | 1061 |
| 915794 | 1260 | 1275 | 19100 | 19115 | TCGCAATGGCAGATTC | 67 | 1062 |
| 915814 | 1333 | 1348 | 23738 | 23753 | TGTGAGGTCACCCACT | 0 | 1063 |
| 915834 | 1360 | 1375 | 23765 | 23780 | AGACACATCAGCACTC | 24 | 1064 |
| 915854 | 1432 | 1447 | 25195 | 25210 | CAGTCCTGCTCAGGTG | 54 | 1065 |
| 915874 | 1526 | 1541 | 25289 | 25304 | GAAGTTCAGGCTGGAC | 75 | 1066 |
| 915894 | 1572 | 1587 | 25335 | 25350 | AGGTGGAGAGCCCCTC | 0 | 1067 |
| 915914 | 1623 | 1638 | 25386 | 25401 | ACTCGCCTCCTCAAGT | 0 | 1068 |
| 915934 | 1661 | 1676 | 25424 | 25439 | AGATGGGAAACTTTAG | 84 | 1069 |
| 915954 | 1724 | 1739 | 25487 | 25502 | GGCTGGGATCCTCCAC | 24 | 1070 |
| 915974 | 1779 | 1794 | 25542 | 25557 | GCTGCACAGGCGAAAG | 56 | 1071 |
| 915994 | 1801 | 1816 | 25564 | 25579 | TATTAGAGTTAAGTGC | 75 | 1072 |
| 916014 | 1828 | 1843 | 25591 | 25606 | AACCAGCTGAATTAAC | 55 | 1073 |
| 916034 | 1875 | 1890 | 25638 | 25653 | CAGTCAGTAAGGGACC | 70 | 1074 |
| 916054 | 1898 | 1913 | 25661 | 25676 | TGACCATTAATAGGGC | 74 | 1075 |
| 916074 | 1925 | 1940 | 25688 | 25703 | TTCTAAGAACCTCATG | 22 | 1076 |
| 916094 | 1968 | 1983 | 25731 | 25746 | CTACCCCCCATCACAA | 0 | 1077 |
| 916114 | 1992 | 2007 | 25755 | 25770 | CCACAAGATCACACAT | 0 | 1078 |
| 916134 | 2087 | 2102 | 25850 | 25865 | CAGACCACCTGACAGG | 78 | 1079 |
| 916153 | 2113 | 2128 | 25876 | 25891 | TTTAGTAGTCAAGGTT | 93 | 1080 |
| 916173 | 2178 | 2193 | 25941 | 25956 | TAGGTGAAAAAGGTGT | 89 | 1081 |
| 916193 | 2306 | 2321 | 26069 | 26084 | ACCCAACCGATTTTTT | 61 | 1082 |
| 916213 | 2636 | 2651 | 26399 | 26414 | CTGAGGTGAATGCCCT | 73 | 1083 |
| 916233 | 2697 | 2712 | 26460 | 26475 | TTGTGTGCTCCAGTGG | 92 | 1084 |
| 916253 | 2746 | 2761 | 26509 | 26524 | TCACTGACCATGTGGG | 16 | 1085 |
| 916273 | N/A | N/A | 3362 | 3377 | CTTCATGCACGGGCGC | 37 | 1086 |
| 916293 | N/A | N/A | 4462 | 4477 | GCATAATCTCCTGCCT | 0 | 1087 |
| 916313 | N/A | N/A | 5284 | 5299 | GCCATAAATCTTGGGA | 37 | 1088 |
| 916333 | N/A | N/A | 5605 | 5620 | CTTTATTCAATGTGGC | 97 | 1089 |
| 916353 | N/A | N/A | 6529 | 6544 | TACAACTGCCTGTGTT | 0 | 1090 |
| 916373 | N/A | N/A | 7218 | 7233 | AAAGCTTCCGCAAACA | 51 | 1091 |
| 916393 | N/A | N/A | 7657 | 7672 | CTAACATACACCCTCG | 0 | 1092 |
| 916553 | N/A | N/A | 12225 | 12240 | AGCTTCTGGGACAAGC | 10 | 1093 |
| 916573 | N/A | N/A | 12746 | 12761 | GGCATACTAAAACCAC | 55 | 1094 |
| 916593 | N/A | N/A | 13397 | 13412 | TTGAATGTCACCCTTC | 91 | 1095 |
| 916613 | N/A | N/A | 13914 | 13929 | AGTCATGACATCCCAG | 93 | 1096 |
| 916633 | N/A | N/A | 14442 | 14457 | TCTCATTGGCACCTGT | 86 | 1097 |
| 916653 | N/A | N/A | 15252 | 15267 | CCCTATCAGATGCCCT | 81 | 1098 |
| 916673 | N/A | N/A | 15799 | 15814 | CATATCTGGTTTCATG | 0 | 1099 |
| 916693 | N/A | N/A | 16842 | 16857 | GACCATAGCACTGTCT | 0 | 1100 |
| 916713 | N/A | N/A | 17737 | 17752 | ATTAATCTGGTCATAT | 0 | 1101 |
| 916733 | N/A | N/A | 18898 | 18913 | TCCATAAAATACGCCC | 69 | 1102 |
| 916753 | N/A | N/A | 20195 | 20210 | GAAAGATGGAATTCCC | 86 | 1103 |
| 916772 | N/A | N/A | 20604 | 20619 | TACGATCATCATTATT | 91 | 1104 |
| 916792 | N/A | N/A | 20841 | 20856 | GTATTAGCTCAATATT | 0 | 1105 |
| 916812 | N/A | N/A | 21314 | 21329 | GTTTACTAGAGACTCT | 64 | 1106 |
| 916832 | N/A | N/A | 22080 | 22095 | GTAAAAACTGTAGGAT | 0 | 1107 |
| 916852 | N/A | N/A | 23220 | 23235 | GATAAATGCCTGACCA | 29 | 1108 |
| 916872 | N/A | N/A | 24011 | 24026 | CCGACGGGAAGTCTTC | 0 | 1109 |
| 916892 | N/A | N/A | 24874 | 24889 | GGCCTATCAGTTTCCC | 0 | 1110 |

TABLE 17
Inhibition of PNPLA3 mRNA by 3-10-3 cEt gapmers targeting SEQ ID NO: 1 and 2

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | PNPLA3 | SEQ |
| Compound | Start | Stop | Start | Stop | | % | ID |
| Number | Site | Site | Site | Site | Sequence (5′ to 3′) | Inhibition | NO |
| 915355 | 37 | 52 | 2775 | 2790 | TCTCGGCCAGGGCATT | 0 | 1111 |
| 915375 | 90 | 105 | 2828 | 2843 | ATCCGCAGCAGCTCCG | 52 | 1112 |
| 915395 | 138 | 153 | 2876 | 2891 | GGCGCGGGTTAGGATC | 0 | 1113 |
| 915415 | 241 | 256 | 2979 | 2994 | CAGCGGGTCGCCCCGA | 8 | 1114 |
| 915435 | 338 | 353 | 3076 | 3091 | GATACCGGAGAGGACG | 30 | 1115 |
| 915455 | 378 | 393 | 5952 | 5967 | GCACAAGATCTGAGAG | 72 | 1116 |
| 915475 | 405 | 420 | 5979 | 5994 | TGCCAATGTTCCGACT | 0 | 1117 |
| 915495 | 443 | 458 | 6017 | 6032 | TCGGAGGAACTTGCTT | 69 | 1118 |
| 915515 | 472 | 487 | 6046 | 6061 | TTGGCCGGGAGGCATT | 9 | 1119 |
| 915535 | 502 | 517 | 6076 | 6091 | CCTATTTTGCCGGAGA | 96 | 1120 |
| 915555 | 524 | 539 | 6098 | 6113 | AGACACTCTGGTAAGA | 2 | 1121 |
| 915615 | 712 | 727 | 11930 | 11945 | GACACGGTGATGGTTG | 32 | 1122 |
| 915635 | 792 | 807 | 12010 | 12025 | GACTGAGCTTGGTGAT | 93 | 1123 |
| 915655 | 839 | 854 | 12057 | 12072 | GACAAAAGCTCTCGAG | 40 | 1124 |
| 915675 | 901 | 916 | 13637 | 13652 | AACCTGAATGCATCCA | 92 | 1125 |
| 915695 | 975 | 990 | 16095 | 16110 | CCTCAGGATCCATCCC | 0 | 1126 |
| 915715 | 1011 | 1026 | 16131 | 16146 | AATCCAGACTCATGTT | 67 | 1127 |
| 915735 | 1088 | 1103 | 16208 | 16223 | CAGGATGCTGAGACGC | 86 | 1128 |
| 915755 | 1144 | 1159 | N/A | N/A | CTCAGTGCTGTAGCGA | 25 | 1129 |
| 915775 | 1209 | 1224 | 19049 | 19064 | TTATCCTAATGGGTAG | 23 | 1130 |
| 915795 | 1261 | 1276 | 19101 | 19116 | ATCGCAATGGCAGATT | 0 | 1131 |
| 915815 | 1337 | 1352 | 23742 | 23757 | CACCTGTGAGGTCACC | 35 | 1132 |
| 915835 | 1361 | 1376 | 23766 | 23781 | CAGACACATCAGCACT | 54 | 1133 |
| 915855 | 1433 | 1448 | 25196 | 25211 | CCAGTCCTGCTCAGGT | 23 | 1134 |
| 915875 | 1530 | 1545 | 25293 | 25308 | AGAAGAAGTTCAGGCT | 81 | 1135 |
| 915895 | 1574 | 1589 | 25337 | 25352 | AAAGGTGGAGAGCCCC | 76 | 1136 |
| 915915 | 1624 | 1639 | 25387 | 25402 | GACTCGCCTCCTCAAG | 75 | 1137 |
| 915935 | 1674 | 1689 | 25437 | 25452 | GGTAGCTGCACAAAGA | 76 | 1138 |
| 915955 | 1726 | 1741 | 25489 | 25504 | GAGGCTGGGATCCTCC | 0 | 1139 |
| 915975 | 1780 | 1795 | 25543 | 25558 | CGCTGCACAGGCGAAA | 0 | 1140 |
| 915995 | 1805 | 1820 | 25568 | 25583 | GATGTATTAGAGTTAA | 82 | 1141 |
| 916015 | 1829 | 1844 | 25592 | 25607 | CAACCAGCTGAATTAA | 59 | 1142 |
| 916035 | 1876 | 1891 | 25639 | 25654 | ACAGTCAGTAAGGGAC | 81 | 1143 |
| 916055 | 1899 | 1914 | 25662 | 25677 | CTGACCATTAATAGGG | 49 | 1144 |
| 916075 | 1929 | 1944 | 25692 | 25707 | GTCATTCTAAGAACCT | 81 | 1145 |
| 916095 | 1969 | 1984 | 25732 | 25747 | CCTACCCCCCATCACA | 21 | 1146 |
| 916115 | 1995 | 2010 | 25758 | 25773 | ACCCCACAAGATCACA | 0 | 1147 |
| 916135 | 2088 | 2103 | 25851 | 25866 | GCAGACCACCTGACAG | 44 | 1148 |
| 916154 | 2131 | 2146 | 25894 | 25909 | CCCCGCCATGGAGACG | 68 | 1149 |
| 916174 | 2180 | 2195 | 25943 | 25958 | GTTAGGTGAAAAAGGT | 90 | 1150 |
| 916194 | 2308 | 2323 | 26071 | 26086 | GCACCCAACCGATTTT | 83 | 1151 |
| 916214 | 2637 | 2652 | 26400 | 26415 | GCTGAGGTGAATGCCC | 52 | 1152 |
| 916234 | 2698 | 2713 | 26461 | 26476 | GTTGTGTGCTCCAGTG | 88 | 1153 |
| 916254 | 2747 | 2762 | 26510 | 26525 | CTCACTGACCATGTGG | 13 | 1154 |
| 916274 | N/A | N/A | 3524 | 3539 | GCAAATCGGCCCCTCG | 3 | 1155 |
| 916294 | N/A | N/A | 4463 | 4478 | GGCATAATCTCCTGCC | 0 | 1156 |
| 916314 | N/A | N/A | 5324 | 5339 | TGGCATGCAAGACCAC | 0 | 1157 |
| 916334 | N/A | N/A | 5606 | 5621 | ACTTTATTCAATGTGG | 95 | 1158 |
| 916354 | N/A | N/A | 6556 | 6571 | GTTTATGTCACTCTGG | 68 | 1159 |
| 916374 | N/A | N/A | 7245 | 7260 | GAACAGACAAGTGCTG | 38 | 1160 |
| 916394 | N/A | N/A | 7658 | 7673 | ACTAACATACACCCTC | 31 | 1161 |
| 916554 | N/A | N/A | 12249 | 12264 | ATAATCAGGGTGGTGC | 0 | 1162 |
| 916574 | N/A | N/A | 12747 | 12762 | AGGCATACTAAAACCA | 47 | 1163 |
| 916594 | N/A | N/A | 13500 | 13515 | GAATCATGCAAGCTCT | 50 | 1164 |
| 916614 | N/A | N/A | 13996 | 14011 | TAAACTAAGGGTCACA | 37 | 1165 |
| 916634 | N/A | N/A | 14497 | 14512 | ATCCATCCTGCATGAG | 76 | 1166 |
| 916654 | N/A | N/A | 15254 | 15269 | GGCCCTATCAGATGCC | 0 | 1167 |
| 916674 | N/A | N/A | 15802 | 15817 | CTACATATCTGGTTTC | 0 | 1168 |
| 916694 | N/A | N/A | 16844 | 16859 | TGGACCATAGCACTGT | 60 | 1169 |
| 916714 | N/A | N/A | 17738 | 17753 | TATTAATCTGGTCATA | 18 | 1170 |
| 916734 | N/A | N/A | 18926 | 18941 | CCACTTTACTCTGTTG | 64 | 1171 |
| 916754 | N/A | N/A | 20210 | 20225 | AACTATGCCTAGAACG | 43 | 1172 |
| 916773 | N/A | N/A | 20606 | 20621 | TTTACGATCATCATTA | 77 | 1173 |
| 916793 | N/A | N/A | 20842 | 20857 | TGTATTAGCTCAATAT | 0 | 1174 |
| 916813 | N/A | N/A | 21319 | 21334 | TGGGAGTTTACTAGAG | 66 | 1175 |
| 916833 | N/A | N/A | 22118 | 22133 | AGAGAGTACTCTTGGA | 11 | 1176 |
| 916853 | N/A | N/A | 23222 | 23237 | CTGATAAATGCCTGAC | 78 | 1177 |
| 916873 | N/A | N/A | 24038 | 24053 | ATCAATGCTGCACTCA | 88 | 1178 |
| 916893 | N/A | N/A | 24889 | 24904 | ACGAATCCCTGGAGGG | 0 | 1179 |

TABLE 18
Inhibition of PNPLA3 mRNA by 3-10-3 cEt gapmers targeting SEQ ID NO: 1 and 2

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | PNPLA3 | SEQ |
| Compound | Start | Stop | Start | Stop | | % | ID |
| Number | Site | Site | Site | Site | Sequence (5′ to 3′) | Inhibition | NO |
| 915356 | 38 | 53 | 2776 | 2791 | GTCTCGGCCAGGGCAT | 0 | 1180 |
| 915376 | 93 | 108 | 2831 | 2846 | CTGATCCGCAGCAGCT | 28 | 1181 |
| 915396 | 165 | 180 | 2903 | 2918 | CGTACATGGCGGCGGC | 0 | 1182 |
| 915416 | 242 | 257 | 2980 | 2995 | GCAGCGGGTCGCCCCG | 0 | 1183 |
| 915436 | 339 | 354 | 3077 | 3092 | GGATACCGGAGAGGAC | 64 | 1184 |
| 915456 | 379 | 394 | 5953 | 5968 | CGCACAAGATCTGAGA | 79 | 1185 |
| 915476 | 406 | 421 | 5980 | 5995 | ATGCCAATGTTCCGAC | 83 | 1186 |
| 915496 | 444 | 459 | 6018 | 6033 | GTCGGAGGAACTTGCT | 35 | 1187 |
| 915516 | 473 | 488 | 6047 | 6062 | ATTGGCCGGGAGGCAT | 0 | 1188 |
| 915536 | 503 | 518 | 6077 | 6092 | GCCTATTTTGCCGGAG | 77 | 1189 |
| 915556 | 525 | 540 | 6099 | 6114 | CAGACACTCTGGTAAG | 62 | 1190 |
| 915616 | 730 | 745 | 11948 | 11963 | TACTCCCCATAGAAGG | 8 | 1191 |
| 915636 | 794 | 809 | 12012 | 12027 | TAGACTGAGCTTGGTG | 90 | 1192 |
| 915656 | 840 | 855 | 12058 | 12073 | GGACAAAAGCTCTCGA | 60 | 1193 |
| 915676 | 902 | 917 | 13638 | 13653 | GAACCTGAATGCATCC | 72 | 1194 |
| 915696 | 978 | 993 | 16098 | 16113 | CGACCTCAGGATCCAT | 0 | 1195 |
| 915716 | 1012 | 1027 | 16132 | 16147 | GAATCCAGACTCATGT | 0 | 1196 |
| 915736 | 1089 | 1104 | 16209 | 16224 | GCAGGATGCTGAGACG | 55 | 1197 |
| 915756 | 1145 | 1160 | N/A | N/A | ACTCAGTGCTGTAGCG | 12 | 1198 |
| 915776 | 1210 | 1225 | 19050 | 19065 | ATTATCCTAATGGGTA | 28 | 1199 |
| 915796 | 1262 | 1277 | 19102 | 19117 | AATCGCAATGGCAGAT | 0 | 1200 |
| 915816 | 1339 | 1354 | 23744 | 23759 | AACACCTGTGAGGTCA | 56 | 1201 |
| 915836 | 1365 | 1380 | 23770 | 23785 | GGAGCAGACACATCAG | 53 | 1202 |
| 915856 | 1434 | 1449 | 25197 | 25212 | GCCAGTCCTGCTCAGG | 21 | 1203 |
| 915876 | 1531 | 1546 | 25294 | 25309 | AAGAAGAAGTTCAGGC | 85 | 1204 |
| 915896 | 1575 | 1590 | 25338 | 25353 | GAAAGGTGGAGAGCCC | 78 | 1205 |
| 915916 | 1626 | 1641 | 25389 | 25404 | TAGACTCGCCTCCTCA | 32 | 1206 |
| 915936 | 1676 | 1691 | 25439 | 25454 | GAGGTAGCTGCACAAA | 91 | 1207 |
| 915956 | 1737 | 1752 | 25500 | 25515 | AACTCAGCTCAGAGGC | 46 | 1208 |
| 915976 | 1781 | 1796 | 25544 | 25559 | CCGCTGCACAGGCGAA | 0 | 1209 |
| 915996 | 1807 | 1822 | 25570 | 25585 | CTGATGTATTAGAGTT | 93 | 1210 |
| 916016 | 1830 | 1845 | 25593 | 25608 | CCAACCAGCTGAATTA | 21 | 1211 |
| 916036 | 1877 | 1892 | 25640 | 25655 | AACAGTCAGTAAGGGA | 82 | 1212 |
| 916056 | 1900 | 1915 | 25663 | 25678 | TCTGACCATTAATAGG | 13 | 1213 |
| 916076 | 1930 | 1945 | 25693 | 25708 | TGTCATTCTAAGAACC | 40 | 1214 |
| 916096 | 1970 | 1985 | 25733 | 25748 | GCCTACCCCCCATCAC | 18 | 1215 |
| 916116 | 1996 | 2011 | 25759 | 25774 | CACCCCACAAGATCAC | 50 | 1216 |
| 916136 | 2089 | 2104 | 25852 | 25867 | TGCAGACCACCTGACA | 58 | 1217 |
| 916155 | 2132 | 2147 | 25895 | 25910 | CCCCCGCCATGGAGAC | 33 | 1218 |
| 916175 | 2224 | 2239 | 25987 | 26002 | CGCTTCCTTACATTTT | 89 | 1219 |
| 916195 | 2309 | 2324 | 26072 | 26087 | TGCACCCAACCGATTT | 64 | 1220 |
| 916215 | 2638 | 2653 | 26401 | 26416 | GGCTGAGGTGAATGCC | 0 | 1221 |
| 916235 | 2699 | 2714 | 26462 | 26477 | AGTTGTGTGCTCCAGT | 85 | 1222 |
| 916255 | 2748 | 2763 | 26511 | 26526 | ACTCACTGACCATGTG | 0 | 1223 |
| 916275 | N/A | N/A | 3555 | 3570 | GGCCAAAGCCCCACTC | 0 | 1224 |
| 916295 | N/A | N/A | 4464 | 4479 | GGGCATAATCTCCTGC | 0 | 1225 |
| 916315 | N/A | N/A | 5342 | 5357 | GGCTGATCTGCACTCT | 84 | 1226 |
| 916335 | N/A | N/A | 5626 | 5641 | TAATTCTACCTGTGTC | 92 | 1227 |
| 916355 | N/A | N/A | 6557 | 6572 | AGTTTATGTCACTCTG | 27 | 1228 |
| 916375 | N/A | N/A | 7321 | 7336 | ACACTTTGCGAAGCAC | 27 | 1229 |
| 916395 | N/A | N/A | 7660 | 7675 | GAACTAACATACACCC | 1 | 1230 |
| 916555 | N/A | N/A | 12252 | 12267 | CCCATAATCAGGGTGG | 0 | 1231 |
| 916575 | N/A | N/A | 12758 | 12773 | GTAGAGTGGTAAGGCA | 95 | 1232 |
| 916595 | N/A | N/A | 13502 | 13517 | AAGAATCATGCAAGCT | 34 | 1233 |
| 916615 | N/A | N/A | 13997 | 14012 | TTAAACTAAGGGTCAC | 65 | 1234 |
| 916635 | N/A | N/A | 14549 | 14564 | TTAATGTGGATTCACG | 76 | 1235 |
| 916655 | N/A | N/A | 15295 | 15310 | CCAAGATAACCTCACA | 64 | 1236 |
| 916675 | N/A | N/A | 15806 | 15821 | CCATCTACATATCTGG | 26 | 1237 |
| 916695 | N/A | N/A | 16854 | 16869 | CACAATCATTTGGACC | 72 | 1238 |
| 916715 | N/A | N/A | 17739 | 17754 | GTATTAATCTGGTCAT | 87 | 1239 |
| 916735 | N/A | N/A | 19113 | 19128 | CACCTCTGGACAATCG | 29 | 1240 |
| 916755 | N/A | N/A | 20212 | 20227 | CAAACTATGCCTAGAA | 70 | 1241 |
| 916774 | N/A | N/A | 20608 | 20623 | ATTTTACGATCATCAT | 91 | 1242 |
| 916794 | N/A | N/A | 20846 | 20861 | TGCCTGTATTAGCTCA | 90 | 1243 |
| 916814 | N/A | N/A | 21345 | 21360 | CACATAAAGTCAAACG | 87 | 1244 |
| 916834 | N/A | N/A | 22124 | 22139 | AGAACAAGAGAGTACT | 3 | 1245 |
| 916854 | N/A | N/A | 23250 | 23265 | CACATAAAGGACCCCC | 54 | 1246 |
| 916874 | N/A | N/A | 24126 | 24141 | CGCTATCTGACACTCC | 87 | 1247 |
| 916894 | N/A | N/A | 24896 | 24911 | TCCACCAACGAATCCC | 50 | 1248 |

TABLE 19
Inhibition of PNPLA3 mRNA by 3-10-3 cEt gapmers targeting SEQ ID NO: 1 and 2

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | PNPLA3 | SEQ |
| Compound | Start | Stop | Start | Stop | | % | ID |
| Number | Site | Site | Site | Site | Sequence (5′ to 3′) | Inhibition | NO |
| 915357 | 39 | 54 | 2777 | 2792 | TGTCTCGGCCAGGGCA | 0 | 1249 |
| 915377 | 94 | 109 | 2832 | 2847 | CCTGATCCGCAGCAGC | 0 | 1250 |
| 915397 | 182 | 197 | 2920 | 2935 | CCAGCCGCGCTCTGCG | 0 | 1251 |
| 915417 | 243 | 258 | 2981 | 2996 | GGCAGCGGGTCGCCCC | 0 | 1252 |
| 915437 | 341 | 356 | 3079 | 3094 | CGGGATACCGGAGAGG | 57 | 1253 |
| 915457 | 380 | 395 | 5954 | 5969 | CCGCACAAGATCTGAG | 71 | 1254 |
| 915477 | 407 | 422 | 5981 | 5996 | GATGCCAATGTTCCGA | 93 | 1255 |
| 915497 | 445 | 460 | 6019 | 6034 | TGTCGGAGGAACTTGC | 85 | 1256 |
| 915517 | 474 | 489 | 6048 | 6063 | CATTGGCCGGGAGGCA | 0 | 1257 |
| 915537 | 504 | 519 | 6078 | 6093 | TGCCTATTTTGCCGGA | 44 | 1258 |
| 915557 | 526 | 541 | 6100 | 6115 | TCAGACACTCTGGTAA | 26 | 1259 |
| 915617 | 732 | 747 | 11950 | 11965 | CGTACTCCCCATAGAA | 66 | 1260 |
| 915637 | 796 | 811 | 12014 | 12029 | CGTAGACTGAGCTTGG | 98 | 1261 |
| 915657 | 857 | 872 | 12075 | 12090 | CACCTTGAGATCCGGG | 0 | 1262 |
| 915677 | 903 | 918 | 13639 | 13654 | AGAACCTGAATGCATC | 78 | 1263 |
| 915697 | 979 | 994 | 16099 | 16114 | GCGACCTCAGGATCCA | 1 | 1264 |
| 915717 | 1031 | 1046 | 16151 | 16166 | GGCAGCCGACTCCGGG | 19 | 1265 |
| 915737 | 1109 | 1124 | 16229 | 16244 | CAGGATGCTCTCATCC | 0 | 1266 |
| 915757 | 1146 | 1161 | N/A | N/A | CACTCAGTGCTGTAGC | 33 | 1267 |
| 915777 | 1214 | 1229 | 19054 | 19069 | AGACATTATCCTAATG | 42 | 1268 |
| 915797 | 1263 | 1278 | 19103 | 19118 | CAATCGCAATGGCAGA | 49 | 1269 |
| 915817 | 1340 | 1355 | 23745 | 23760 | GAACACCTGTGAGGTC | 44 | 1270 |
| 915837 | 1398 | 1413 | 25161 | 25176 | GGCTGCTCACTGGCAT | 18 | 1271 |
| 915857 | 1435 | 1450 | 25198 | 25213 | GGCCAGTCCTGCTCAG | 0 | 1272 |
| 915877 | 1534 | 1549 | 25297 | 25312 | CCCAAGAAGAAGTTCA | 76 | 1273 |
| 915897 | 1576 | 1591 | 25339 | 25354 | GGAAAGGTGGAGAGCC | 24 | 1274 |
| 915917 | 1627 | 1642 | 25390 | 25405 | CTAGACTCGCCTCCTC | 77 | 1275 |
| 915937 | 1679 | 1694 | 25442 | 25457 | GCGGAGGTAGCTGCAC | 16 | 1276 |
| 915957 | 1738 | 1753 | 25501 | 25516 | CAACTCAGCTCAGAGG | 61 | 1277 |
| 915977 | 1782 | 1797 | 25545 | 25560 | ACCGCTGCACAGGCGA | 34 | 1278 |
| 915997 | 1809 | 1824 | 25572 | 25587 | TGCTGATGTATTAGAG | 83 | 1279 |
| 916017 | 1831 | 1846 | 25594 | 25609 | CCCAACCAGCTGAATT | 42 | 1280 |
| 916037 | 1878 | 1893 | 25641 | 25656 | AAACAGTCAGTAAGGG | 92 | 1281 |
| 916057 | 1901 | 1916 | 25664 | 25679 | GTCTGACCATTAATAG | 64 | 1282 |
| 916077 | 1931 | 1946 | 25694 | 25709 | CTGTCATTCTAAGAAC | 41 | 1283 |
| 916097 | 1971 | 1986 | 25734 | 25749 | AGCCTACCCCCCATCA | 0 | 1284 |
| 916117 | 1997 | 2012 | 25760 | 25775 | CCACCCCACAAGATCA | 0 | 1285 |
| 916137 | 2090 | 2105 | 25853 | 25868 | TTGCAGACCACCTGAC | 65 | 1286 |
| 916156 | 2133 | 2148 | 25896 | 25911 | ACCCCCGCCATGGAGA | 54 | 1287 |
| 916176 | 2225 | 2240 | 25988 | 26003 | ACGCTTCCTTACATTT | 84 | 1288 |
| 916196 | 2310 | 2325 | 26073 | 26088 | CTGCACCCAACCGATT | 58 | 1289 |
| 916216 | 2639 | 2654 | 26402 | 26417 | GGGCTGAGGTGAATGC | 46 | 1290 |
| 916236 | 2700 | 2715 | 26463 | 26478 | AAGTTGTGTGCTCCAG | 86 | 1291 |
| 916256 | 2751 | 2766 | 26514 | 26529 | GAAACTCACTGACCAT | 41 | 1292 |
| 916276 | N/A | N/A | 4068 | 4083 | GGAAACAACTTTCCTC | 0 | 1293 |
| 916296 | N/A | N/A | 4730 | 4745 | GATCATGTGGCGGTCT | 68 | 1294 |
| 916316 | N/A | N/A | 5364 | 5379 | CACTTACTGGCCTGGC | 30 | 1295 |
| 916336 | N/A | N/A | 5645 | 5660 | ATATTGGGCTCAATGA | 89 | 1296 |
| 916356 | N/A | N/A | 6575 | 6590 | ATCACTGGAGGTGTAC | 0 | 1297 |
| 916376 | N/A | N/A | 7328 | 7343 | CAGGATCACACTTTGC | 17 | 1298 |
| 916396 | N/A | N/A | 7661 | 7676 | GGAACTAACATACACC | 0 | 1299 |
| 916556 | N/A | N/A | 12272 | 12287 | GTATATGTTCCCAGGT | 81 | 1300 |
| 916576 | N/A | N/A | 12788 | 12803 | GTGTACATGGTCTGCA | 94 | 1301 |
| 916596 | N/A | N/A | 13529 | 13544 | ATCATTGGAAGACCGC | 89 | 1302 |
| 916616 | N/A | N/A | 13998 | 14013 | GTTAAACTAAGGGTCA | 85 | 1303 |
| 916636 | N/A | N/A | 14550 | 14565 | CTTAATGTGGATTCAC | 91 | 1304 |
| 916656 | N/A | N/A | 15351 | 15366 | TCCAACTTCAGGCTGA | 74 | 1305 |
| 916676 | N/A | N/A | 15819 | 15834 | AGCTTTGTGGGCTCCA | 69 | 1306 |
| 916696 | N/A | N/A | 16982 | 16997 | GTTTAATAAGGGCACC | 63 | 1307 |
| 916716 | N/A | N/A | 17740 | 17755 | CGTATTAATCTGGTCA | 93 | 1308 |
| 916736 | N/A | N/A | 19126 | 19141 | CACCTAAAATGCTCAC | 17 | 1309 |
| 916756 | N/A | N/A | 20213 | 20228 | ACAAACTATGCCTAGA | 58 | 1310 |
| 916775 | N/A | N/A | 20609 | 20624 | AATTTTACGATCATCA | 78 | 1311 |
| 916795 | N/A | N/A | 20927 | 20942 | GACAGATCAGCACTCG | 80 | 1312 |
| 916815 | N/A | N/A | 21407 | 21422 | CAATTCTAGACATGGC | 88 | 1313 |
| 916835 | N/A | N/A | 22338 | 22353 | TGCACCTACCCTTTTC | 39 | 1314 |
| 916855 | N/A | N/A | 23251 | 23266 | ACACATAAAGGACCCC | 48 | 1315 |
| 916875 | N/A | N/A | 24241 | 24256 | GCATTACCAGGCACCT | 61 | 1316 |
| 916895 | N/A | N/A | 24912 | 24927 | GACATCACAGGTGTTG | 5 | 1317 |

TABLE 20
Inhibition of PNPLA3 mRNA by 3-10-3 cEt gapmers targeting SEQ ID NO: 1 and 2

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | PNPLA3 | SEQ |
| Compound | Start | Stop | Start | Stop | | % | ID |
| Number | Site | Site | Site | Site | Sequence (5′ to 3′) | Inhibition | NO |
| 915358 | 40 | 55 | 2778 | 2793 | GTGTCTCGGCCAGGGC | 0 | 1318 |
| 915378 | 96 | 111 | 2834 | 2849 | GTCCTGATCCGCAGCA | 0 | 1319 |
| 915398 | 184 | 199 | 2922 | 2937 | CTCCAGCCGCGCTCTG | 14 | 1320 |
| 915418 | 244 | 259 | 2982 | 2997 | AGGCAGCGGGTCGCCC | 0 | 1321 |
| 915438 | 342 | 357 | 3080 | 3095 | GCGGGATACCGGAGAG | 44 | 1322 |
| 915458 | 381 | 396 | 5955 | 5970 | TCCGCACAAGATCTGA | 41 | 1323 |
| 915478 | 408 | 423 | 5982 | 5997 | AGATGCCAATGTTCCG | 95 | 1324 |
| 915498 | 446 | 461 | 6020 | 6035 | CTGTCGGAGGAACTTG | 40 | 1325 |
| 915518 | 475 | 49 | 6049 | 6064 | ACATTGGCCGGGAGGC | 5 | 1326 |
| 915538 | 505 | 520 | 6079 | 6094 | ATGCCTATTTTGCCGG | 61 | 1327 |
| 915558 | 527 | 542 | 6101 | 6116 | ATCAGACACTCTGGTA | 0 | 1328 |
| 915618 | 748 | 763 | 11966 | 11981 | ACTTTAGGGCAGATGT | 87 | 1329 |
| 915638 | 818 | 833 | 12036 | 12051 | GTAGAGGTTCCCTGTG | 87 | 1330 |
| 915658 | 859 | 874 | N/A | N/A | AGCACCTTGAGATCCG | 0 | 1331 |
| 915678 | 926 | 941 | N/A | N/A | GTTGCAGATGCCCTTC | 24 | 1332 |
| 915698 | 980 | 995 | 16100 | 16115 | GGCGACCTCAGGATCC | 0 | 1333 |
| 915718 | 1032 | 1047 | 16152 | 16167 | AGGCAGCCGACTCCGG | 8 | 1334 |
| 915738 | 1114 | 1129 | 16234 | 16249 | GTGTCCAGGATGCTCT | 41 | 1335 |
| 915758 | 1147 | 1162 | N/A | N/A | TCACTCAGTGCTGTAG | 59 | 1336 |
| 915778 | 1217 | 1232 | 19057 | 19072 | ATAAGACATTATCCTA | 85 | 1337 |
| 915798 | 1264 | 1279 | 19104 | 19119 | ACAATCGCAATGGCAG | 66 | 1338 |
| 915818 | 1342 | 1357 | 23747 | 23762 | GTGAACACCTGTGAGG | 58 | 1339 |
| 915838 | 1400 | 1415 | 25163 | 25178 | TTGGCTGCTCACTGGC | 79 | 1340 |
| 915858 | 1436 | 1451 | 25199 | 25214 | GGGCCAGTCCTGCTCA | 0 | 1341 |
| 915878 | 1535 | 1550 | 25298 | 25313 | GCCCAAGAAGAAGTTC | 54 | 1342 |
| 915898 | 1590 | 1605 | 25353 | 25368 | CTAGTGAAAAACTGGG | 34 | 1343 |
| 915918 | 1628 | 1643 | 25391 | 25406 | GCTAGACTCGCCTCCT | 33 | 1344 |
| 915938 | 1680 | 1695 | 25443 | 25458 | TGCGGAGGTAGCTGCA | 0 | 1345 |
| 915958 | 1756 | 1771 | 25519 | 25534 | CCTAGCTTTTCATAAA | 0 | 1346 |
| 915978 | 1783 | 1798 | 25546 | 25561 | GACCGCTGCACAGGCG | 24 | 1347 |
| 915998 | 1810 | 1825 | 25573 | 25588 | ATGCTGATGTATTAGA | 86 | 1348 |
| 916018 | 1832 | 1847 | 25595 | 25610 | TCCCAACCAGCTGAAT | 3 | 1349 |
| 916038 | 1879 | 1894 | 25642 | 25657 | GAAACAGTCAGTAAGG | 64 | 1350 |
| 916058 | 1902 | 1917 | 25665 | 25680 | AGTCTGACCATTAATA | 86 | 1351 |
| 916078 | 1933 | 1948 | 25696 | 25711 | ACCTGTCATTCTAAGA | 18 | 1352 |
| 916098 | 1972 | 1987 | 25735 | 25750 | CAGCCTACCCCCCATC | 41 | 1353 |
| 916118 | 1999 | 2014 | 25762 | 25777 | CTCCACCCCACAAGAT | 0 | 1354 |
| 916138 | 2092 | 2107 | 25855 | 25870 | CTTTGCAGACCACCTG | 65 | 1355 |
| 916157 | 2134 | 2149 | 25897 | 25912 | TACCCCCGCCATGGAG | 57 | 1356 |
| 916177 | 2237 | 2252 | 26000 | 26015 | CAACAGGTAACAACGC | 88 | 1357 |
| 916197 | 2579 | 2594 | 26342 | 26357 | GTCAGACTTTCACTCA | 81 | 1358 |
| 916217 | 2659 | 2674 | 26422 | 26437 | GTGCTTGGCTCCTGCC | 43 | 1359 |
| 916237 | 2701 | 2716 | 26464 | 26479 | CAAGTTGTGTGCTCCA | 73 | 1360 |
| 916257 | 2769 | 2784 | 26532 | 26547 | CATCGCCACACATGGG | 61 | 1361 |
| 916277 | N/A | N/A | 4105 | 4120 | AGGAAGGGTCCCAAAC | 0 | 1362 |
| 916297 | N/A | N/A | 4731 | 4746 | TGATCATGTGGCGGTC | 80 | 1363 |
| 916317 | N/A | N/A | 5391 | 5406 | TGCTATCAGGTGCAGG | 60 | 1364 |
| 916337 | N/A | N/A | 5646 | 5661 | TATATTGGGCTCAATG | 71 | 1365 |
| 916357 | N/A | N/A | 6594 | 6609 | GTTTACAAACATGGAC | 26 | 1366 |
| 916377 | N/A | N/A | 7464 | 7479 | TCATTAGCATCACCGG | 33 | 1367 |
| 916397 | N/A | N/A | 7662 | 7677 | GGGAACTAACATACAC | 0 | 1368 |
| 916557 | N/A | N/A | 12274 | 12289 | GGGTATATGTTCCCAG | 0 | 1369 |
| 916577 | N/A | N/A | 12830 | 12845 | TGCATAGCCTTCTTTC | 84 | 1370 |
| 916597 | N/A | N/A | 13530 | 13545 | CATCATTGGAAGACCG | 62 | 1371 |
| 916617 | N/A | N/A | 14016 | 14031 | TCTTTAACTTCGGCCC | 70 | 1372 |
| 916637 | N/A | N/A | 14551 | 14566 | TCTTAATGTGGATTCA | 88 | 1373 |
| 916657 | N/A | N/A | 15388 | 15403 | TCAGACAACCACAGCT | 66 | 1374 |
| 916677 | N/A | N/A | 15852 | 15867 | TAAAGCAGGACACACG | 74 | 1375 |
| 916697 | N/A | N/A | 17077 | 17092 | AGACATGTTGGTGTCT | 0 | 1376 |
| 916717 | N/A | N/A | 17788 | 17803 | CCCCAGTCTTTTATTC | 0) | 1377 |
| 916737 | N/A | N/A | 19140 | 19155 | GGAAGACACGGAGCCA | 20 | 1378 |
| 916757 | N/A | N/A | 20240 | 20255 | CCTAACTGCTGGCTCT | 85 | 1379 |
| 916776 | N/A | N/A | 20610 | 20625 | TAATTTTACGATCATC | 76 | 1380 |
| 916796 | N/A | N/A | 20939 | 20954 | CTCTTTGTAGCAGACA | 90 | 1381 |
| 916816 | N/A | N/A | 21439 | 21454 | CAATATACTGAGAGGA | 92 | 1382 |
| 916836 | N/A | N/A | 22392 | 22407 | GTAGACATCCTTCCCG | 65 | 1383 |
| 916856 | N/A | N/A | 23252 | 23267 | GACACATAAAGGACCC | 59 | 1384 |
| 916876 | N/A | N/A | 24242 | 24257 | TGCATTACCAGGCACC | 37 | 1385 |
| 916896 | N/A | N/A | 24913 | 24928 | GGACATCACAGGTGTT | 19 | 1386 |

TABLE 21
Inhibition of PNPLA3 mRNA by 3-10-3 cEt gapmers targeting SEQ ID NO: 1 and 2

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | PNPLA3 | SEQ |
| Compound | Start | Stop | Start | Stop | | % | ID |
| Number | Site | Site | Site | Site | Sequence (5′ to 3′) | Inhibition | NO |
| 915359 | 41 | 56 | 2779 | 2794 | AGTGTCTCGGCCAGGG | 0 | 1387 |
| 915379 | 97 | 112 | 2835 | 2850 | GGTCCTGATCCGCAGC | 0 | 1388 |
| 915399 | 185 | 200 | 2923 | 2938 | GCTCCAGCCGCGCTCT | 26 | 1389 |
| 915419 | 245 | 260 | 2983 | 2998 | CAGGCAGCGGGTCGCC | 0 | 1390 |
| 915439 | 343 | 358 | 3081 | 3096 | AGCGGGATACCGGAGA | 69 | 1391 |
| 915459 | 382 | 397 | 5956 | 5971 | TTCCGCACAAGATCTG | 71 | 1392 |
| 915479 | 409 | 424 | 5983 | 5998 | AAGATGCCAATGTTCC | 93 | 1393 |
| 915499 | 448 | 463 | 6022 | 6037 | CCCTGTCGGAGGAACT | 30 | 1394 |
| 915519 | 477 | 492 | 6051 | 6066 | GGACATTGGCCGGGAG | 88 | 1395 |
| 915539 | 506 | 521 | 6080 | 6095 | GATGCCTATTTTGCCG | 60 | 1396 |
| 915559 | 529 | 544 | 6103 | 6118 | CCATCAGACACTCTGG | 30 | 1397 |
| 915579 | 595 | 610 | 7825 | 7840 | CAGGAACATACCAAGG | 98 | 1398 |
| 915599 | 674 | 689 | 11892 | 11907 | GTTGTCACTCACTCCT | 98 | 1399 |
| 915619 | 749 | 764 | 11967 | 11982 | GACTTTAGGGCAGATG | 96 | 1400 |
| 915639 | 819 | 834 | 12037 | 12052 | GGTAGAGGTTCCCTGT | 92 | 1401 |
| 915659 | 860 | 875 | N/A | N/A | CAGCACCTTGAGATCC | 0 | 1402 |
| 915679 | 928 | 943 | N/A | N/A | CTGTTGCAGATGCCCT | 58 | 1403 |
| 915699 | 981 | 996 | 16101 | 16116 | TGGCGACCTCAGGATC | 40 | 1404 |
| 915719 | 1033 | 1048 | 16153 | 16168 | AAGGCAGCCGACTCCG | 0 | 1405 |
| 915739 | 1115 | 1130 | 16235 | 16250 | GGTGTCCAGGATGCTC | 40 | 1406 |
| 915759 | 1148 | 1163 | N/A | N/A | TTCACTCAGTGCTGTA | 29 | 1407 |
| 915779 | 1219 | 1234 | 19059 | 19074 | ACATAAGACATTATCC | 86 | 1408 |
| 915799 | 1268 | 1283 | 19108 | 19123 | CTGGACAATCGCAATG | 42 | 1409 |
| 915819 | 1344 | 1359 | 23749 | 23764 | GAGTGAACACCTGTGA | 81 | 1410 |
| 915839 | 1401 | 1416 | 25164 | 25179 | GTTGGCTGCTCACTGG | 85 | 1411 |
| 915859 | 1437 | 1452 | 25200 | 25215 | AGGGCCAGTCCTGCTC | 0 | 1412 |
| 915879 | 1538 | 1553 | 25301 | 25316 | ATTGCCCAAGAAGAAG | 54 | 1413 |
| 915899 | 1591 | 1606 | 25354 | 25369 | TCTAGTGAAAAACTGG | 0 | 1414 |
| 915919 | 1629 | 1644 | 25392 | 25407 | TGCTAGACTCGCCTCC | 72 | 1415 |
| 915939 | 1681 | 1696 | 25444 | 25459 | ATGCGGAGGTAGCTGC | 39 | 1416 |
| 915959 | 1764 | 1779 | 25527 | 25542 | GGTTGCTTCCTAGCTT | 87 | 1417 |
| 915979 | 1784 | 1799 | 25547 | 25562 | GGACCGCTGCACAGGC | 0 | 1418 |
| 915999 | 1811 | 1826 | 25574 | 25589 | CATGCTGATGTATTAG | 35 | 1419 |
| 916019 | 1833 | 1848 | 25596 | 25611 | TTCCCAACCAGCTGAA | 0 | 1420 |
| 916039 | 1880 | 1895 | 25643 | 25658 | CGAAACAGTCAGTAAG | 80 | 1421 |
| 916059 | 1905 | 1920 | 25668 | 25683 | AACAGTCTGACCATTA | 85 | 1422 |
| 916079 | 1934 | 1949 | 25697 | 25712 | CACCTGTCATTCTAAG | 45 | 1423 |
| 916099 | 1973 | 1988 | 25736 | 25751 | CCAGCCTACCCCCCAT | 53 | 1424 |
| 916119 | 2022 | 2037 | 25785 | 25800 | GTGGGATCATGCTATT | 76 | 1425 |
| 916139 | 2093 | 2108 | 25856 | 25871 | TCTTTGCAGACCACCT | 85 | 1426 |
| 916158 | 2135 | 2150 | 25898 | 25913 | TTACCCCCGCCATGGA | 0 | 1427 |
| 916178 | 2238 | 2253 | 26001 | 26016 | TCAACAGGTAACAACG | 87 | 1428 |
| 916198 | 2620 | 2635 | 26383 | 26398 | GCACACTAGATTATTT | 66 | 1429 |
| 916218 | 2673 | 2688 | 26436 | 26451 | CGGAAGCTCCTGCTGT | 27 | 1430 |
| 916238 | 2702 | 2717 | 26465 | 26480 | TCAAGTTGTGTGCTCC | 91 | 1431 |
| 916258 | 2770 | 2785 | 26533 | 26548 | TCATCGCCACACATGG | 49 | 1432 |
| 916278 | N/A | N/A | 4211 | 4226 | TCATTTCCAGGAGTAC | 75 | 1433 |
| 916298 | N/A | N/A | 4735 | 4750 | CAAATGATCATGTGGC | 93 | 1434 |
| 916318 | N/A | N/A | 5394 | 5409 | TAATGCTATCAGGTGC | 95 | 1435 |
| 916338 | N/A | N/A | 5648 | 5663 | GATATATTGGGCTCAA | 97 | 1436 |
| 916358 | N/A | N/A | 6596 | 6611 | GGGTTTACAAACATGG | 75 | 1437 |
| 916378 | N/A | N/A | 7465 | 7480 | TTCATTAGCATCACCG | 78 | 1438 |
| 916398 | N/A | N/A | 7686 | 7701 | GTTAATCCATGGGTCA | 49 | 1439 |
| 916418 | N/A | N/A | 8992 | 9007 | AGCCTAAACTTCCTCC | 63 | 1440 |
| 916438 | N/A | N/A | 9318 | 9333 | AGAAGAGCCGCCCTGC | 77 | 1441 |
| 916458 | N/A | N/A | 9795 | 9810 | GCAAGACTAGCAAGTG | 85 | 1442 |
| 916478 | N/A | N/A | 10301 | 10316 | AGCATGCGGTATGTAC | 67 | 1443 |
| 916498 | N/A | N/A | 10849 | 10864 | CACACAATTTCTAGGG | 82 | 1444 |
| 916518 | N/A | N/A | 11346 | 11361 | TTGACAATTAGAACCA | 96 | 1445 |
| 916538 | N/A | N/A | 11711 | 11726 | ACAAATCCTTACCGAG | 54 | 1446 |
| 916558 | N/A | N/A | 12285 | 12300 | GTTTTAGGTCTGGGTA | 94 | 1447 |
| 916578 | N/A | N/A | 12831 | 12846 | TTGCATAGCCTTCTTT | 93 | 1448 |
| 916598 | N/A | N/A | 13660 | 13675 | CATACATACCCTTCTC | 9 | 1449 |
| 916618 | N/A | N/A | 14025 | 14040 | CGCAGAAACTCTTTAA | 89 | 1450 |
| 916638 | N/A | N/A | 14552 | 14567 | GTCTTAATGTGGATTC | 93 | 1451 |
| 916658 | N/A | N/A | 15421 | 15436 | AGCATTGGCACACTGG | 70 | 1452 |
| 916678 | N/A | N/A | 15857 | 15872 | GGCTTTAAAGCAGGAC | 62 | 1453 |
| 916698 | N/A | N/A | 17079 | 17094 | GCAGACATGTTGGTGT | 2 | 1454 |
| 916718 | N/A | N/A | 17839 | 17854 | TACAAGCTGGTCCTTG | 0 | 1455 |
| 916738 | N/A | N/A | 19211 | 19226 | GACAATCCAGGTCCCA | 70 | 1456 |
| 916758 | N/A | N/A | 20285 | 20300 | GAGGAAGCCCAATCAA | 81 | 1457 |
| 916777 | N/A | N/A | 20611 | 20626 | CTAATTTTACGATCAT | 81 | 1458 |
| 916797 | N/A | N/A | 20984 | 20999 | TTAAACTGCCAAGTCC | 83 | 1459 |
| 916817 | N/A | N/A | 21440 | 21455 | CCAATATACTGAGAGG | 96 | 1460 |
| 916837 | N/A | N/A | 22406 | 22421 | GGTAGCACCGCCAAGT | 0 | 1461 |
| 916857 | N/A | N/A | 23301 | 23316 | CACCATGGAGAGGTCT | 0 | 1462 |
| 916877 | N/A | N/A | 24243 | 24258 | TTGCATTACCAGGCAC | 17 | 1463 |
| 916897 | N/A | N/A | 24934 | 24949 | GCTACCTGGACACCTC | 47 | 1464 |

TABLE 22
Inhibition of PNPLA3 mRNA by 3-10-3 cEt gapmers targeting SEQ ID NO: 1 and 2

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | PNPLA3 | SEQ |
| Compound | Start | Stop | Start | Stop | | % | ID |
| Number | Site | Site | Site | Site | Sequence (5′ to 3′) | Inhibition | NO |
| 841947 | 2094 | 2109 | 25857 | 25872 | ATCTTTGCAGACCACC | 89 | 1464 |
| 912986 | N/A | N/A | 20288 | 20303 | TCAGAGGAAGCCCAAT | 92 | 254 |
| | | | 20318 | 20333 | | | |
| 915360 | 42 | 57 | 2780 | 2795 | CAGTGTCTCGGCCAGG | 0 | 1466 |
| 915380 | 98 | 113 | 2836 | 2851 | GGGTCCTGATCCGCAG | 0 | 1467 |
| 915400 | 186 | 201 | 2924 | 2939 | AGCTCCAGCCGCGCTC | 0 | 1468 |
| 915420 | 246 | 261 | 2984 | 2999 | TCAGGCAGCGGGTCGC | 78 | 1469 |
| 915440 | 344 | 359 | 3082 | 3097 | CAGCGGGATACCGGAG | 72 | 1470 |
| 915460 | 383 | 398 | 5957 | 5972 | CTTCCGCACAAGATCT | 0 | 1471 |
| 915480 | 411 | 426 | 5985 | 6000 | GGAAGATGCCAATGTT | 94 | 1472 |
| 915500 | 449 | 464 | 6023 | 6038 | ACCCTGTCGGAGGAAC | 40 | 1473 |
| 915520 | 480 | 495 | 6054 | 6069 | GGTGGACATTGGCCGG | 38 | 1474 |
| 915540 | 507 | 522 | 6081 | 6096 | AGATGCCTATTTTGCC | 76 | 1475 |
| 915560 | 556 | 571 | 6130 | 6145 | CGAAAGTCAGACACCA | 69 | 1476 |
| 915620 | 750 | 765 | 11968 | 11983 | TGACTTTAGGGCAGAT | 89 | 1477 |
| 915640 | 821 | 836 | 12039 | 12054 | AAGGTAGAGGTTCCCT | 10 | 1478 |
| 915660 | 875 | 890 | 13611 | 13626 | AAGGCATATCTCTCCC | 47 | 1479 |
| 915680 | 929 | 944 | N/A | N/A | CCTGTTGCAGATGCCC | 22 | 1480 |
| 915700 | 982 | 997 | 16102 | 16117 | ATGGCGACCTCAGGAT | 58 | 1481 |
| 915720 | 1034 | 1049 | 16154 | 16169 | CAAGGCAGCCGACTCC | 63 | 1482 |
| 915740 | 1121 | 1136 | 16241 | 16256 | CGAGAGGGTGTCCAGG | 0 | 1483 |
| 915760 | 1149 | 1164 | N/A | N/A | CTTCACTCAGTGCTGT | 13 | 1484 |
| 915780 | 1226 | 1241 | 19066 | 19081 | CAGCATTACATAAGAC | 94 | 1485 |
| 915800 | 1270 | 1285 | 19110 | 19125 | CTCTGGACAATCGCAA | 55 | 1486 |
| 915820 | 1345 | 1360 | 23750 | 23765 | CGAGTGAACACCTGTG | 83 | 1487 |
| 915840 | 1402 | 1417 | 25165 | 25180 | TGTTGGCTGCTCACTG | 77 | 1488 |
| 915860 | 1470 | 1485 | 25233 | 25248 | CTGGACAGCCCTTGGG | 29 | 1489 |
| 915880 | 1539 | 1554 | 25302 | 25317 | TATTGCCCAAGAAGAA | 18 | 1490 |
| 915900 | 1598 | 1613 | 25361 | 25376 | ACTCTTCTCTAGTGAA | 67 | 1491 |
| 915920 | 1630 | 1645 | 25393 | 25408 | CTGCTAGACTCGCCTC | 88 | 1492 |
| 915940 | 1682 | 1697 | 25445 | 25460 | AATGCGGAGGTAGCTG | 0 | 1493 |
| 915960 | 1765 | 1780 | 25528 | 25543 | AGGTTGCTTCCTAGCT | 55 | 1494 |
| 915980 | 1785 | 1800 | 25548 | 25563 | TGGACCGCTGCACAGG | 82 | 1495 |
| 916000 | 1812 | 1827 | 25575 | 25590 | GCATGCTGATGTATTA | 52 | 1496 |
| 916020 | 1837 | 1852 | 25600 | 25615 | TCATTTCCCAACCAGC | 94 | 1497 |
| 916040 | 1881 | 1896 | 25644 | 25659 | ACGAAACAGTCAGTAA | 79 | 1498 |
| 916060 | 1907 | 1922 | 25670 | 25685 | GGAACAGTCTGACCAT | 22 | 1499 |
| 916080 | 1936 | 1951 | 25699 | 25714 | AACACCTGTCATTCTA | 71 | 1500 |
| 916100 | 1974 | 1989 | 25737 | 25752 | GCCAGCCTACCCCCCA | 23 | 1501 |
| 916120 | 2023 | 2038 | 25786 | 25801 | AGTGGGATCATGCTAT | 0 | 1502 |
| 916159 | 2136 | 2151 | 25899 | 25914 | GTTACCCCCGCCATGG | 47 | 1503 |
| 916179 | 2239 | 2254 | 26002 | 26017 | TTCAACAGGTAACAAC | 84 | 1504 |
| 916199 | 2621 | 2636 | 26384 | 26399 | TGCACACTAGATTATT | 0 | 1505 |
| 916219 | 2674 | 2689 | 26437 | 26452 | GCGGAAGCTCCTGCTG | 6 | 1506 |
| 916239 | 2704 | 2719 | 26467 | 26482 | GTTCAAGTTGTGTGCT | 85 | 1507 |
| 916259 | 2771 | 2786 | 26534 | 26549 | CTCATCGCCACACATG | 85 | 1508 |
| 916279 | N/A | N/A | 4218 | 4233 | CGGAATCTCATTTCCA | 0 | 1509 |
| 916299 | N/A | N/A | 4736 | 4751 | GCAAATGATCATGTGG | 93 | 1510 |
| 916319 | N/A | N/A | 5396 | 5411 | CTTAATGCTATCAGGT | 83 | 1511 |
| 916339 | N/A | N/A | 5649 | 5664 | GGATATATTGGGCTCA | 96 | 1512 |
| 916359 | N/A | N/A | 6597 | 6612 | AGGGTTTACAAACATG | 32 | 1513 |
| 916379 | N/A | N/A | 7466 | 7481 | ATTCATTAGCATCACC | 52 | 1514 |
| 916399 | N/A | N/A | 7687 | 7702 | GGTTAATCCATGGGTC | 0 | 1515 |
| 916559 | N/A | N/A | 12286 | 12301 | AGTTTTAGGTCTGGGT | 89 | 1516 |
| 916579 | N/A | N/A | 12833 | 12848 | CATTGCATAGCCTTCT | 96 | 1517 |
| 916599 | N/A | N/A | 13661 | 13676 | CCATACATACCCTTCT | 21 | 1518 |
| 916619 | N/A | N/A | 14077 | 14092 | ACCCACACCTGACTGG | 16 | 1519 |
| 916639 | N/A | N/A | 14572 | 14587 | CGCTCCTACTTATCCC | 96 | 1520 |
| 916659 | N/A | N/A | 15427 | 15442 | TCTTACAGCATTGGCA | 38 | 1521 |
| 916679 | N/A | N/A | 15973 | 15988 | CATCTACCAAACTGCA | 73 | 1522 |
| 916699 | N/A | N/A | 17135 | 17150 | AACAAACATCGATTTT | 47 | 1523 |
| 916719 | N/A | N/A | 17844 | 17859 | AGCTTTACAAGCTGGT | 0 | 1524 |
| 916739 | N/A | N/A | 19213 | 19228 | ACGACAATCCAGGTCC | 14 | 1525 |
| 916778 | N/A | N/A | 20612 | 20627 | TCTAATTTTACGATCA | 92 | 1526 |
| 916798 | N/A | N/A | 20985 | 21000 | ATTAAACTGCCAAGTC | 72 | 1527 |
| 916818 | N/A | N/A | 21441 | 21456 | ACCAATATACTGAGAG | 92 | 1528 |
| 916838 | N/A | N/A | 22409 | 22424 | AGCGGTAGCACCGCCA | 0 | 1529 |
| 916858 | N/A | N/A | 23323 | 23338 | TCACATGTGAGCCCAG | 46 | 1530 |
| 916878 | N/A | N/A | 24271 | 24286 | GTACAACAGAGGGTGG | 19 | 1531 |
| 916898 | N/A | N/A | 24978 | 24993 | GGCTGATGTCACCACC | 32 | 1532 |

TABLE 23
Inhibition of PNPLA3 mRNA by 3-10-3 cEt gapmers targeting SEQ ID NO: 1 and 2

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | PNPLA3 | SEQ |
| Compound | Start | Stop | Start | Stop | | % | ID |
| Number | Site | Site | Site | Site | Sequence (5′ to 3′) | Inhibition | NO |
| 915361 | 43 | 58 | 2781 | 2796 | TCAGTGTCTCGGCCAG | 0 | 1533 |
| 915381 | 105 | 120 | 2843 | 2858 | TCGGCTCGGGTCCTGA | 0 | 1534 |
| 915401 | 188 | 203 | 2926 | 2941 | CAAGCTCCAGCCGCGC | 22 | 1535 |
| 915421 | 247 | 262 | 2985 | 3000 | CTCAGGCAGCGGGTCG | 48 | 1536 |
| 915441 | 345 | 360 | 3083 | 3098 | CCAGCGGGATACCGGA | 0 | 1537 |
| 915461 | 384 | 399 | 5958 | 5973 | CCTTCCGCACAAGATC | 59 | 1538 |
| 915481 | 414 | 429 | 5988 | 6003 | GATGGAAGATGCCAAT | 83 | 1539 |
| 915501 | 450 | 465 | 6024 | 6039 | GACCCTGTCGGAGGAA | 26 | 1540 |
| 915521 | 482 | 497 | 6056 | 6071 | CTGGTGGACATTGGCC | 0 | 1541 |
| 915541 | 508 | 523 | 6082 | 6097 | GAGATGCCTATTTTGC | 92 | 1542 |
| 915561 | 557 | 572 | 6131 | 6146 | CCGAAAGTCAGACACC | 0 | 1543 |
| 915601 | 691 | 706 | 11909 | 11924 | GCATCAATGAAGGGTA | 91 | 1544 |
| 915621 | 751 | 766 | 11969 | 11984 | TTGACTTTAGGGCAGA | 85 | 1545 |
| 915641 | 822 | 837 | 12040 | 12055 | GAAGGTAGAGGTTCCC | 74 | 1546 |
| 915661 | 876 | 891 | 13612 | 13627 | GAAGGCATATCTCTCC | 32 | 1547 |
| 915681 | 930 | 945 | 16050 | 16065 | GCCTGTTGCAGATGCC | 0 | 1548 |
| 915701 | 983 | 998 | 16103 | 16118 | CATGGCGACCTCAGGA | 0 | 1549 |
| 915721 | 1035 | 1050 | 16155 | 16170 | CCAAGGCAGCCGACTC | 79 | 1550 |
| 915741 | 1122 | 1137 | 16242 | 16257 | GCGAGAGGGTGTCCAG | 0 | 1551 |
| 915761 | 1150 | 1165 | 18990 | 19005 | TCTTCACTCAGTGCTG | 41 | 1552 |
| 915781 | 1227 | 1242 | 19067 | 19082 | GCAGCATTACATAAGA | 75 | 1553 |
| 915801 | 1273 | 1288 | N/A | N/A | AGTCTCTGGACAATCG | 0 | 1554 |
| 915821 | 1346 | 1361 | 23751 | 23766 | TCGAGTGAACACCTGT | 52 | 1555 |
| 915841 | 1403 | 1418 | 25166 | 25181 | CTGTTGGCTGCTCACT | 80 | 1556 |
| 915861 | 1471 | 1486 | 25234 | 25249 | GCTGGACAGCCCTTGG | 0 | 1557 |
| 915881 | 1540 | 1555 | 25303 | 25318 | TTATTGCCCAAGAAGA | 75 | 1558 |
| 915901 | 1599 | 1614 | 25362 | 25377 | GACTCTTCTCTAGTGA | 67 | 1559 |
| 915921 | 1631 | 1646 | 25394 | 25409 | TCTGCTAGACTCGCCT | 50 | 1560 |
| 915941 | 1683 | 1698 | 25446 | 25461 | CAATGCGGAGGTAGCT | 39 | 1561 |
| 915961 | 1766 | 1781 | 25529 | 25544 | AAGGTTGCTTCCTAGC | 71 | 1562 |
| 915981 | 1786 | 1801 | 25549 | 25564 | CTGGACCGCTGCACAG | 0 | 1563 |
| 916001 | 1813 | 1828 | 25576 | 25591 | CGCATGCTGATGTATT | 73 | 1564 |
| 916021 | 1840 | 1855 | 25603 | 25618 | GTGTCATTTCCCAACC | 61 | 1565 |
| 916041 | 1882 | 1897 | 25645 | 25660 | CACGAAACAGTCAGTA | 34 | 1566 |
| 916061 | 1910 | 1925 | 25673 | 25688 | GCTGGAACAGTCTGAC | 82 | 1567 |
| 916081 | 1942 | 1957 | 25705 | 25720 | CATCCAAACACCTGTC | 69 | 1568 |
| 916101 | 1975 | 1990 | 25738 | 25753 | GGCCAGCCTACCCCCC | 2 | 1569 |
| 916121 | 2024 | 2039 | 25787 | 25802 | AAGTGGGATCATGCTA | 26 | 1570 |
| 916140 | 2095 | 2110 | 25858 | 25873 | CATCTTTGCAGACCAC | 92 | 1571 |
| 916160 | 2137 | 2152 | 25900 | 25915 | TGTTACCCCCGCCATG | 51 | 1572 |
| 916180 | 2257 | 2272 | 26020 | 26035 | GATTCACATAATACAA | 87 | 1573 |
| 916200 | 2622 | 2637 | 26385 | 26400 | CTGCACACTAGATTAT | 0 | 1574 |
| 916220 | 2675 | 2690 | 26438 | 26453 | GGCGGAAGCTCCTGCT | 0 | 1575 |
| 916240 | 2705 | 2720 | 26468 | 26483 | GGTTCAAGTTGTGTGC | 69 | 1576 |
| 916260 | 2772 | 2787 | 26535 | 26550 | TCTCATCGCCACACAT | 72 | 1577 |
| 916280 | N/A | N/A | 4220 | 4235 | TACGGAATCTCATTTC | 80 | 1578 |
| 916300 | N/A | N/A | 4791 | 4806 | GGCCACCTTGGGATAC | 17 | 1579 |
| 916320 | N/A | N/A | 5398 | 5413 | GCCTTAATGCTATCAG | 67 | 1580 |
| 916340 | N/A | N/A | 5650 | 5665 | TGGATATATTGGGCTC | 97 | 1581 |
| 916360 | N/A | N/A | 6603 | 6618 | ACATTCAGGGTTTACA | 18 | 1582 |
| 916380 | N/A | N/A | 7468 | 7483 | GTATTCATTAGCATCA | 51 | 1583 |
| 916400 | N/A | N/A | 7688 | 7703 | AGGTTAATCCATGGGT | 29 | 1584 |
| 916560 | N/A | N/A | 12287 | 12302 | GAGTTTTAGGTCTGGG | 95 | 1585 |
| 916580 | N/A | N/A | 12905 | 12920 | CTTATAAAGCACACGG | 95 | 1586 |
| 916600 | N/A | N/A | 13683 | 13698 | GGGCATGGCTGATCCT | 8 | 1587 |
| 916620 | N/A | N/A | 14099 | 14114 | CAAACTTGTCTAGTGG | 67 | 1588 |
| 916640 | N/A | N/A | 14600 | 14615 | TCGCATCCATGGGTCC | 83 | 1589 |
| 916660 | N/A | N/A | 15429 | 15444 | GCTCTTACAGCATTGG | 51 | 1590 |
| 916680 | N/A | N/A | 15974 | 15989 | CCATCTACCAAACTGC | 61 | 1591 |
| 916700 | N/A | N/A | 17195 | 17210 | GACTTAGTCCGTGTTC | 49 | 1592 |
| 916720 | N/A | N/A | 17883 | 17898 | CAGCATCTATGTTCTC | 67 | 1593 |
| 916740 | N/A | N/A | 19239 | 19254 | ATAGACTGTGAGCTGT | 82 | 1594 |
| 916759 | N/A | N/A | 20354 | 20369 | GACCATTCTGCTCCCC | 20 | 1595 |
| 916779 | N/A | N/A | 20632 | 20647 | GCCCATACCTTTTATC | 47 | 1596 |
| 916799 | N/A | N/A | 20987 | 21002 | GTATTAAACTGCCAAG | 81 | 1597 |
| 916819 | N/A | N/A | 21444 | 21459 | CTAACCAATATACTGA | 73 | 1598 |
| 916839 | N/A | N/A | 22506 | 22521 | GGCTGGTGATGAAACA | 0 | 1599 |
| 916859 | N/A | N/A | 23345 | 23360 | CCTCATGGTTTGCTGT | 31 | 1600 |
| 916879 | N/A | N/A | 24273 | 24288 | CAGTACAACAGAGGGT | 81 | 1601 |
| 916899 | N/A | N/A | 25064 | 25079 | CACATTGCCGGCCAGT | 58 | 1602 |

TABLE 24
Inhibition of PNPLA3 mRNA by 3-10-3 cEt gapmers targeting SEQ ID NO: 1 and 2

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | PNPLA3 | SEQ |
| Compound | Start | Stop | Start | Stop | | % | ID |
| Number | Site | Site | Site | Site | Sequence (5′ to 3′) | Inhibition | NO |
| 915362 | 44 | 59 | 2782 | 2797 | CTCAGTGTCTCGGCCA | 0 | 1603 |
| 915382 | 106 | 121 | 2844 | 2859 | ATCGGCTCGGGTCCTG | 30 | 1604 |
| 915402 | 189 | 204 | 2927 | 2942 | ACAAGCTCCAGCCGCG | 24 | 1605 |
| 915422 | 248 | 263 | 2986 | 3001 | GCTCAGGCAGCGGGTC | 33 | 1606 |
| 915442 | 346 | 361 | N/A | N/A | TCCAGCGGGATACCGG | 0 | 1607 |
| 915462 | 385 | 400 | 5959 | 5974 | GCCTTCCGCACAAGAT | 60 | 1608 |
| 915482 | 415 | 430 | 5989 | 6004 | GGATGGAAGATGCCAA | 60 | 1609 |
| 915502 | 451 | 466 | 6025 | 6040 | AGACCCTGTCGGAGGA | 75 | 1610 |
| 915522 | 487 | 502 | 6061 | 6076 | ATGAGCTGGTGGACAT | 74 | 1611 |
| 915542 | 509 | 524 | 6083 | 6098 | AGAGATGCCTATTTTG | 91 | 1612 |
| 915562 | 558 | 573 | 6132 | 6147 | ACCGAAAGTCAGACAC | 0 | 1613 |
| 915602 | 692 | 707 | 11910 | 11925 | GGCATCAATGAAGGGT | 88 | 1614 |
| 915622 | 752 | 767 | 11970 | 11985 | CTTGACTTTAGGGCAG | 86 | 1615 |
| 915642 | 824 | 839 | 12042 | 12057 | GAGAAGGTAGAGGTTC | 81 | 1616 |
| 915662 | 878 | 893 | 13614 | 13629 | TCGAAGGCATATCTCT | 16 | 1617 |
| 915682 | 931 | 946 | 16051 | 16066 | GGCCTGTTGCAGATGC | 0 | 1618 |
| 915702 | 984 | 999 | 16104 | 16119 | GCATGGCGACCTCAGG | 24 | 1619 |
| 915722 | 1036 | 1051 | 16156 | 16171 | GCCAAGGCAGCCGACT | 0 | 1620 |
| 915742 | 1123 | 1138 | 16243 | 16258 | GGCGAGAGGGTGTCCA | 0 | 1621 |
| 915762 | 1173 | 1188 | 19013 | 19028 | TGTATCCACCTTTGTC | 85 | 1622 |
| 915782 | 1228 | 1243 | 19068 | 19083 | GGCAGCATTACATAAG | 73 | 1623 |
| 915802 | 1283 | 1298 | N/A | N/A | CCATGTCACCAGTCTC | 59 | 1624 |
| 915822 | 1347 | 1362 | 23752 | 23767 | CTCGAGTGAACACCTG | 0 | 1625 |
| 915842 | 1404 | 1419 | 25167 | 25182 | CCTGTTGGCTGCTCAC | 88 | 1626 |
| 915862 | 1472 | 1487 | 25235 | 25250 | TGCTGGACAGCCCTTG | 0 | 1627 |
| 915882 | 1541 | 1556 | 25304 | 25319 | TTTATTGCCCAAGAAG | 38 | 1628 |
| 915902 | 1600 | 1615 | 25363 | 25378 | AGACTCTTCTCTAGTG | 60 | 1629 |
| 915922 | 1632 | 1647 | 25395 | 25410 | ATCTGCTAGACTCGCC | 86 | 1630 |
| 915942 | 1684 | 1699 | 25447 | 25462 | GCAATGCGGAGGTAGC | 38 | 1631 |
| 915962 | 1767 | 1782 | 25530 | 25545 | AAAGGTTGCTTCCTAG | 77 | 1632 |
| 915982 | 1787 | 1802 | 25550 | 25565 | GCTGGACCGCTGCACA | 79 | 1633 |
| 916002 | 1814 | 1829 | 25577 | 25592 | ACGCATGCTGATGTAT | 72 | 1634 |
| 916022 | 1841 | 1856 | 25604 | 25619 | GGTGTCATTTCCCAAC | 80 | 1635 |
| 916042 | 1883 | 1898 | 25646 | 25661 | CCACGAAACAGTCAGT | 87 | 1636 |
| 916062 | 1912 | 1927 | 25675 | 25690 | ATGCTGGAACAGTCTG | 78 | 1637 |
| 916082 | 1943 | 1958 | 25706 | 25721 | CCATCCAAACACCTGT | 64 | 1638 |
| 916102 | 1976 | 1991 | 25739 | 25754 | GGGCCAGCCTACCCCC | 0 | 1639 |
| 916122 | 2025 | 2040 | 25788 | 25803 | GAAGTGGGATCATGCT | 71 | 1640 |
| 916141 | 2097 | 2112 | 25860 | 25875 | ATCATCTTTGCAGACC | 91 | 1641 |
| 916161 | 2138 | 2153 | 25901 | 25916 | TTGTTACCCCCGCCAT | 89 | 1642 |
| 916181 | 2259 | 2274 | 26022 | 26037 | CTGATTCACATAATAC | 91 | 1643 |
| 916201 | 2623 | 2638 | 26386 | 26401 | CCTGCACACTAGATTA | 59 | 1644 |
| 916221 | 2676 | 2691 | 26439 | 26454 | AGGCGGAAGCTCCTGC | 0 | 1645 |
| 916241 | 2706 | 2721 | 26469 | 26484 | AGGTTCAAGTTGTGTG | 87 | 1646 |
| 916281 | N/A | N/A | 4224 | 4239 | AATGTACGGAATCTCA | 83 | 1647 |
| 916301 | N/A | N/A | 4810 | 4825 | GTCCATGTGGGTGTCC | 74 | 1648 |
| 916321 | N/A | N/A | 5399 | 5414 | GGCCTTAATGCTATCA | 13 | 1649 |
| 916341 | N/A | N/A | 5711 | 5726 | TAGTATGAAATATCTC | 96 | 1650 |
| 916361 | N/A | N/A | 6862 | 6877 | ATTGTAACTGCCAGGC | 0 | 1651 |
| 916381 | N/A | N/A | 7471 | 7486 | CCGGTATTCATTAGCA | 0 | 1652 |
| 916401 | N/A | N/A | 7728 | 7743 | GAGCAGGGCAACAAAC | 22 | 1653 |
| 916561 | N/A | N/A | 12315 | 12330 | ATATAACCACAGCCTG | 54 | 1654 |
| 916581 | N/A | N/A | 12906 | 12921 | GCTTATAAAGCACACG | 94 | 1655 |
| 916601 | N/A | N/A | 13702 | 13717 | TAGTAAATGCTTGTCA | 95 | 1656 |
| 916621 | N/A | N/A | 14123 | 14138 | GGCAGAAATGTGCTCT | 60 | 1657 |
| 916641 | N/A | N/A | 14632 | 14647 | CTTCATGCCATCCTGT | 83 | 1658 |
| 916661 | N/A | N/A | 15430 | 15445 | TGCTCTTACAGCATTG | 0 | 1659 |
| 916681 | N/A | N/A | 16262 | 16277 | GGTACCTGTAGCGAGC | 0 | 1660 |
| 916701 | N/A | N/A | 17197 | 17212 | TTGACTTAGTCCGTGT | 93 | 1661 |
| 916721 | N/A | N/A | 18220 | 18235 | AGCTACATCAGGCTGG | 0 | 1662 |
| 916741 | N/A | N/A | 19244 | 19259 | TGCACATAGACTGTGA | 0 | 1663 |
| 916760 | N/A | N/A | 20373 | 20388 | GACTGCTGAGCCAAGC | 61 | 1664 |
| 916780 | N/A | N/A | 20657 | 20672 | AGAAATTGCAGTGCCC | 92 | 1665 |
| 916800 | N/A | N/A | 20988 | 21003 | GGTATTAAACTGCCAA | 0 | 1666 |
| 916820 | N/A | N/A | 21447 | 21462 | TCACTAACCAATATAC | 47 | 1667 |
| 916840 | N/A | N/A | 22602 | 22617 | ATAAATCTGCAAGAGC | 62 | 1668 |
| 916860 | N/A | N/A | 23369 | 23384 | TCTCATGGTCAAGACC | 52 | 1669 |
| 916880 | N/A | N/A | 24305 | 24320 | GACTGCTAGGCTTCAC | 54 | 1670 |
| 916900 | N/A | N/A | 25100 | 25115 | CGCTGCTGCAGTGTGC | 34 | 1671 |

Human primer probe set RTS36075 (forward sequence TGAGGCTGGAGGGAGATG, designated herein as SEQ ID NO: 14; reverse sequence GCTCATGTATCCACCTTTGTCT, designated herein as SEQ ID NO: 15; probe sequence CTAGACCACCTGCGTCTCAGCATC, designated herein as SEQ ID NO: 16) was also used to measure mRNA levels. PNPLA3 mRNA levels were adjusted according to total RNA content, as measured by RIBOGREENR. Results are presented as percent inhibition of PNPLA3, relative to untreated control cells.

TABLE 25
Inhibition of PNPLA3 mRNA by 3-10-3 cEt gapmers targeting SEQ ID NO: 1 and 2

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | PNPLA3 | SEQ |
| Compound | Start | Stop | Start | Stop | | % | ID |
| Number | Site | Site | Site | Site | Sequence (5′ to 3′) | Inhibition | NO |
| 898558 | 581 | 596 | N/A | N/A | GGCATCCACGACTTCG | 87 | 1672 |
| 912709 | 27 | 42 | 2765 | 2780 | GGCATTCCCAGCGCGA | 0 | 17 |
| 912710 | 95 | 110 | 2833 | 2848 | TCCTGATCCGCAGCAG | 0 | 18 |
| 912711 | 103 | 118 | 2841 | 2856 | GGCTCGGGTCCTGATC | 0 | 19 |
| 912712 | 131 | 146 | 2869 | 2884 | GTTAGGATCTGGGTCG | 76 | 20 |
| 912713 | 164 | 179 | 2902 | 2917 | GTACATGGCGGCGGCG | 0 | 21 |
| 912714 | 183 | 198 | 2921 | 2936 | TCCAGCCGCGCTCTGC | 29 | 22 |
| 912715 | 196 | 211 | 2934 | 2949 | GCGAAGGACAAGCTCC | 31 | 23 |
| 912716 | 197 | 212 | 2935 | 2950 | CGCGAAGGACAAGCTC | 0 | 24 |
| 912717 | 272 | 287 | 3010 | 3025 | GCGGAGGAGGTGCGGG | 0 | 25 |
| 912718 | 273 | 288 | 3011 | 3026 | CGCGGAGGAGGTGCGG | 0 | 26 |
| 912719 | 274 | 289 | 3012 | 3027 | TCGCGGAGGAGGTGCG | 16 | 27 |
| 912720 | 290 | 305 | 3028 | 3043 | GAACAACATGCGCGCG | 0 | 28 |
| 912721 | 291 | 306 | 3029 | 3044 | CGAACAACATGCGCGC | 2 | 29 |
| 912722 | 292 | 307 | 3030 | 3045 | CCGAACAACATGCGCG | 0 | 30 |
| 912723 | 293 | 308 | 3031 | 3046 | GCCGAACAACATGCGC | 0 | 31 |
| 912724 | 294 | 309 | 3032 | 3047 | CGCCGAACAACATGCG | 0 | 32 |
| 912725 | 323 | 338 | 3061 | 3076 | GCCGACGCAGTGCAAC | 0 | 33 |
| 912726 | 324 | 339 | 3062 | 3077 | CGCCGACGCAGTGCAA | 0 | 34 |
| 912727 | 340 | 355 | 3078 | 3093 | GGGATACCGGAGAGGA | 32 | 35 |
| 912728 | 370 | 385 | 5944 | 5959 | TCTGAGAGGACCTGCA | 31 | 36 |
| 912729 | 375 | 390 | 5949 | 5964 | CAAGATCTGAGAGGAC | 60 | 37 |
| 912730 | 404 | 419 | 5978 | 5993 | GCCAATGTTCCGACTC | 52 | 38 |
| 912731 | 410 | 425 | 5984 | 5999 | GAAGATGCCAATGTTC | 31 | 39 |
| 912732 | 429 | 444 | 6003 | 6018 | TTAAGTTGAAGGATGG | 93 | 40 |
| 912733 | 432 | 447 | 6006 | 6021 | TGCTTAAGTTGAAGGA | 82 | 41 |
| 912734 | 478 | 493 | 6052 | 6067 | TGGACATTGGCCGGGA | 73 | 42 |
| 912735 | 479 | 494 | 6053 | 6068 | GTGGACATTGGCCGGG | 44 | 43 |
| 912736 | 484 | 499 | 6058 | 6073 | AGCTGGTGGACATTGG | 29 | 44 |
| 912737 | 528 | 543 | 6102 | 6117 | CATCAGACACTCTGGT | 0 | 45 |
| 912738 | 531 | 546 | 6105 | 6120 | CCCCATCAGACACTCT | 55 | 46 |
| 912739 | 552 | 567 | 6126 | 6141 | AGTCAGACACCAGAAC | 23 | 47 |
| 912740 | 582 | 597 | N/A | N/A | AGGCATCCACGACTTC | 40 | 1673 |
| 912741 | 584 | 599 | N/A | N/A | CAAGGCATCCACGACT | 55 | 1674 |
| 912742 | 591 | 606 | N/A | N/A | AACATACCAAGGCATC | 59 | 1675 |
| 912743 | 593 | 608 | N/A | N/A | GGAACATACCAAGGCA | 69 | 1676 |
| 912744 | 594 | 609 | 7824 | 7839 | AGGAACATACCAAGGC | 85 | 1677 |
| 912745 | 625 | 640 | 7855 | 7870 | GGGATAAGGCCACTGT | 71 | 1678 |
| 912746 | 626 | 641 | 7856 | 7871 | AGGGATAAGGCCACTG | 12 | 1679 |
| 912747 | 630 | 645 | 7860 | 7875 | AAGGAGGGATAAGGCC | 0 | 1680 |
| 912748 | 652 | 667 | N/A | N/A | ACATATCGCACGCCTC | 35 | 1681 |
| 912749 | 653 | 668 | N/A | N/A | CACATATCGCACGCCT | 3 | 1682 |
| 912750 | 654 | 669 | N/A | N/A | CCACATATCGCACGCC | 27 | 1683 |
| 912751 | 656 | 671 | N/A | N/A | ATCCACATATCGCACG | 24 | 1684 |
| 912752 | 660 | 675 | 11878 | 11893 | CTCCATCCACATATCG | 87 | 1685 |
| 912753 | 689 | 704 | 11907 | 11922 | ATCAATGAAGGGTACG | 79 | 1686 |
| 912754 | 690 | 705 | 11908 | 11923 | CATCAATGAAGGGTAC | 63 | 1687 |
| 912755 | 693 | 708 | 11911 | 11926 | TGGCATCAATGAAGGG | 68 | 48 |
| 912756 | 698 | 713 | 11916 | 11931 | TGTTTTGGCATCAATG | 88 | 49 |
| 912757 | 746 | 761 | 11964 | 11979 | TTTAGGGCAGATGTCG | 75 | 50 |
| 912758 | 747 | 762 | 11965 | 11980 | CTTTAGGGCAGATGTC | 82 | 51 |
| 912759 | 795 | 810 | 12013 | 12028 | GTAGACTGAGCTTGGT | 96 | 52 |
| 912760 | 820 | 835 | 12038 | 12053 | AGGTAGAGGTTCCCTG | 0 | 53 |
| 912761 | 841 | 856 | 12059 | 12074 | GGGACAAAAGCTCTCG | 0 | 54 |
| 912762 | 873 | 888 | 13609 | 13624 | GGCATATCTCTCCCAG | 0 | 55 |
| 912763 | 874 | 889 | 13610 | 13625 | AGGCATATCTCTCCCA | 0 | 56 |
| 912764 | 886 | 901 | 13622 | 13637 | AAATATCCTCGAAGGC | 71 | 57 |
| 912765 | 888 | 903 | 13624 | 13639 | CCAAATATCCTCGAAG | 37 | 58 |
| 912766 | 889 | 904 | 13625 | 13640 | TCCAAATATCCTCGAA | 0 | 59 |
| 912767 | 894 | 909 | 13630 | 13645 | ATGCATCCAAATATCC | 42 | 60 |
| 912768 | 925 | 940 | N/A | N/A | TTGCAGATGCCCTTCT | 5 | 61 |
| 912769 | 968 | 983 | 16088 | 16103 | ATCCATCCCTTCTGAG | 6 | 62 |
| 912770 | 986 | 1001 | 16106 | 16121 | GGGCATGGCGACCTCA | 0 | 63 |
| 912771 | 1004 | 1019 | 16124 | 16139 | ACTCATGTTTGCCCAG | 67 | 64 |
| 912782 | 1195 | 1210 | 19035 | 19050 | AGCAAGTTGCAAATCT | 71 | 75 |
| 912783 | 1199 | 1214 | 19039 | 19054 | GGGTAGCAAGTTGCAA | 37 | 76 |
| 912784 | 1205 | 1220 | 19045 | 19060 | CCTAATGGGTAGCAAG | 25 | 77 |
| 912785 | 1206 | 1221 | 19046 | 19061 | TCCTAATGGGTAGCAA | 64 | 78 |

TABLE 26
Inhibition of PNPLA3 mRNA by 3-10-3 cEt gapmers targeting SEQ ID NO: 1 and 2

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | PNPLA3 | SEQ |
| Compound | Start | Stop | Start | Stop | | % | ID |
| Number | Site | Site | Site | Site | Sequence (5′ to 3′) | Inhibition | NO |
| 912786 | 1207 | 1222 | 19047 | 19062 | ATCCTAATGGGTAGCA | 65 | 79 |
| 912787 | 1211 | 1226 | 19051 | 19066 | CATTATCCTAATGGGT | 43 | 80 |
| 912788 | 1212 | 1227 | 19052 | 19067 | ACATTATCCTAATGGG | 0 | 81 |
| 912789 | 1213 | 1228 | 19053 | 19068 | GACATTATCCTAATGG | 59 | 82 |
| 912790 | 1220 | 1235 | 19060 | 19075 | TACATAAGACATTATC | 8 | 83 |
| 912791 | 1224 | 1239 | 19064 | 19079 | GCATTACATAAGACAT | 86 | 84 |
| 912792 | 1245 | 1260 | 19085 | 19100 | CCACAGGCAGGGTACA | 58 | 85 |
| 912793 | 1246 | 1261 | 19086 | 19101 | TCCACAGGCAGGGTAC | 5 | 86 |
| 912794 | 1253 | 1268 | 19093 | 19108 | GGCAGATTCCACAGGC | 68 | 87 |
| 912795 | 1259 | 1274 | 19099 | 19114 | CGCAATGGCAGATTCC | 84 | 88 |
| 912796 | 1265 | 1280 | 19105 | 19120 | GACAATCGCAATGGCA | 63 | 89 |
| 912797 | 1266 | 1281 | 19106 | 19121 | GGACAATCGCAATGGC | 54 | 90 |
| 912798 | 1267 | 1282 | 19107 | 19122 | TGGACAATCGCAATGG | 59 | 91 |
| 912799 | 1285 | 1300 | 23690 | 23705 | AGCCATGTCACCAGTC | 51 | 92 |
| 912800 | 1289 | 1304 | 23694 | 23709 | TGGAAGCCATGTCACC | 32 | 93 |
| 912801 | 1290 | 1305 | 23695 | 23710 | CTGGAAGCCATGTCAC | 44 | 94 |
| 912802 | 1297 | 1312 | 23702 | 23717 | GGCATATCTGGAAGCC | 0 | 95 |
| 912803 | 1298 | 1313 | 23703 | 23718 | GGGCATATCTGGAAGC | 0 | 96 |
| 912804 | 1351 | 1366 | 23756 | 23771 | AGCACTCGAGTGAACA | 6 | 97 |
| 912805 | 1386 | 1401 | N/A | N/A | GCATTTGGGACCTGGA | 54 | 98 |
| 912806 | 1387 | 1402 | N/A | N/A | GGCATTTGGGACCTGG | 33 | 99 |
| 912807 | 1388 | 1403 | 25151 | 25166 | TGGCATTTGGGACCTG | 0 | 100 |
| 912808 | 1394 | 1409 | 25157 | 25172 | GCTCACTGGCATTTGG | 7 | 101 |
| 912809 | 1523 | 1538 | 25286 | 25301 | GTTCAGGCTGGACCTG | 17 | 102 |
| 912810 | 1547 | 1562 | 25310 | 25325 | AGGTACTTTATTGCCC | 30 | 103 |
| 912811 | 1550 | 1565 | 25313 | 25328 | AGCAGGTACTTTATTG | 55 | 104 |
| 912812 | 1653 | 1668 | 25416 | 25431 | AACTTTAGCACCTCTG | 87 | 105 |
| 912813 | 1655 | 1670 | 25418 | 25433 | GAAACTTTAGCACCTC | 85 | 106 |
| 912814 | 1656 | 1671 | 25419 | 25434 | GGAAACTTTAGCACCT | 26 | 107 |
| 912815 | 1669 | 1684 | 25432 | 25447 | CTGCACAAAGATGGGA | 66 | 108 |
| 912816 | 1671 | 1686 | 25434 | 25449 | AGCTGCACAAAGATGG | 41 | 109 |
| 912817 | 1685 | 1700 | 25448 | 25463 | AGCAATGCGGAGGTAG | 35 | 110 |
| 912818 | 1740 | 1755 | 25503 | 25518 | ACCAACTCAGCTCAGA | 76 | 111 |
| 912819 | 1741 | 1756 | 25504 | 25519 | AACCAACTCAGCTCAG | 77 | 112 |
| 912820 | 1757 | 1772 | 25520 | 25535 | TCCTAGCTTTTCATAA | 18 | 113 |
| 912821 | 1788 | 1803 | 25551 | 25566 | TGCTGGACCGCTGCAC | 1 | 114 |
| 912822 | 1796 | 1811 | 25559 | 25574 | GAGTTAAGTGCTGGAC | 90 | 115 |
| 912823 | 1802 | 1817 | 25565 | 25580 | GTATTAGAGTTAAGTG | 86 | 116 |
| 912824 | 1803 | 1818 | 25566 | 25581 | TGTATTAGAGTTAAGT | 79 | 117 |
| 912825 | 1806 | 1821 | 25569 | 25584 | TGATGTATTAGAGTTA | 89 | 118 |
| 912826 | 1808 | 1823 | 25571 | 25586 | GCTGATGTATTAGAGT | 79 | 119 |
| 912827 | 1821 | 1836 | 25584 | 25599 | TGAATTAACGCATGCT | 73 | 120 |
| 912828 | 1822 | 1837 | 25585 | 25600 | CTGAATTAACGCATGC | 69 | 121 |
| 912829 | 1870 | 1885 | 25633 | 25648 | AGTAAGGGACCCTCTG | 0 | 122 |
| 912830 | 1871 | 1886 | 25634 | 25649 | CAGTAAGGGACCCTCT | 44 | 123 |
| 912831 | 1872 | 1887 | 25635 | 25650 | TCAGTAAGGGACCCTC | 67 | 124 |
| 912832 | 1874 | 1889 | 25637 | 25652 | AGTCAGTAAGGGACCC | 50 | 125 |
| 912833 | 1893 | 1908 | 25656 | 25671 | ATTAATAGGGCCACGA | 78 | 126 |
| 912834 | 1895 | 1910 | 25658 | 25673 | CCATTAATAGGGCCAC | 72 | 127 |
| 912835 | 1896 | 1911 | 25659 | 25674 | ACCATTAATAGGGCCA | 65 | 128 |
| 912836 | 1906 | 1921 | 25669 | 25684 | GAACAGTCTGACCATT | 82 | 129 |
| 912837 | 1908 | 1923 | 25671 | 25686 | TGGAACAGTCTGACCA | 39 | 130 |
| 912838 | 1909 | 1924 | 25672 | 25687 | CTGGAACAGTCTGACC | 84 | 131 |
| 912839 | 1911 | 1926 | 25674 | 25689 | TGCTGGAACAGTCTGA | 72 | 132 |
| 912840 | 1916 | 1931 | 25679 | 25694 | CCTCATGCTGGAACAG | 84 | 133 |
| 912841 | 1928 | 1943 | 25691 | 25706 | TCATTCTAAGAACCTC | 87 | 134 |
| 912842 | 1945 | 1960 | 25708 | 25723 | ACCCATCCAAACACCT | 18 | 135 |
| 912843 | 1982 | 1997 | 25745 | 25760 | ACACATGGGCCAGCCT | 46 | 136 |
| 912844 | 1989 | 2004 | 25752 | 25767 | CAAGATCACACATGGG | 71 | 137 |
| 912845 | 2057 | 2072 | 25820 | 25835 | GGGACGAACTGCACCC | 0 | 138 |
| 912846 | 2098 | 2113 | 25861 | 25876 | TATCATCTTTGCAGAC | 68 | 139 |
| 912847 | 2116 | 2131 | 25879 | 25894 | GTTTTTAGTAGTCAAG | 90 | 140 |
| 912848 | 2117 | 2132 | 25880 | 25895 | CGTTTTTAGTAGTCAA | 94 | 141 |
| 912849 | 2145 | 2160 | 25908 | 25923 | TATCATCTTGTTACCC | 87 | 142 |
| 912850 | 2148 | 2163 | 25911 | 25926 | GATTATCATCTTGTTA | 60 | 143 |
| 912851 | 2150 | 2165 | 25913 | 25928 | TAGATTATCATCTTGT | 50 | 144 |
| 912852 | 2151 | 2166 | 25914 | 25929 | GTAGATTATCATCTTG | 72 | 145 |
| 912853 | 2152 | 2167 | 25915 | 25930 | AGTAGATTATCATCTT | 79 | 146 |
| 912854 | 2175 | 2190 | 25938 | 25953 | GTGAAAAAGGTGTTCT | 64 | 147 |
| 912855 | 2182 | 2197 | 25945 | 25960 | TAGTTAGGTGAAAAAG | 77 | 148 |
| 912856 | 2188 | 2203 | 25951 | 25966 | TTATTTTAGTTAGGTG | 82 | 149 |
| 912857 | 2190 | 2205 | 25953 | 25968 | CATTATTTTAGTTAGG | 77 | 150 |
| 912858 | 2273 | 2288 | 26036 | 26051 | CTACTAACATCTCACT | 48 | 151 |
| 912859 | 2274 | 2289 | 26037 | 26052 | TCTACTAACATCTCAC | 91 | 152 |
| 912860 | 2278 | 2293 | 26041 | 26056 | TTATTCTACTAACATC | 37 | 153 |
| 912861 | 2280 | 2295 | 26043 | 26058 | GCTTATTCTACTAACA | 77 | 154 |
| 912862 | 2281 | 2296 | 26044 | 26059 | GGCTTATTCTACTAAC | 70 | 155 |
| 912863 | 2632 | 2647 | 26395 | 26410 | GGTGAATGCCCTGCAC | 42 | 156 |

### Study 2

Cultured A431 cells at a density of 5,000 cells per well were transfected by free uptake with 1,000 nM antisense oligonucleotide. After a treatment period of approximately 24 hours, RNA was isolated from the cells and PNPLA3 mRNA levels were measured by quantitative real-time PCR. Human primer probe set RTS36070 was used to measure mRNA levels. PNPLA3 mRNA levels were adjusted according to total RNA content, as measured by RIBOGREEN R. Results are presented as percent inhibition of PNPLA3, relative to untreated control cells.

TABLE 27
Inhibition of PNPLA3 mRNA by 3-10-3 cEt gapmers targeting SEQ ID NO: 1 and 2

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | PNPLA3 | SEQ |
| Compound | Start | Stop | Start | Stop | Sequence | % | ID |
| Number | Site | Site | Site | Site | (5′ to 3′) | Inhibition | NO |
| 915609 | 705 | 720 | 11923 | 11938 | TGATGGTTGTTTTGGC | 97 | 702 |
| 959270 | 413 | 428 | 5987 | 6002 | ATGGAAGATGCCAATG | 32 | 1688 |
| 959280 | 491 | 506 | 6065 | 6080 | GGAGATGAGCTGGTGG | 66 | 1689 |
| 959290 | 793 | 808 | 12011 | 12026 | AGACTGAGCTTGGTGA | 78 | 1690 |
| 959300 | 899 | 914 | 13635 | 13650 | CCTGAATGCATCCAAA | 69 | 1691 |
| 959310 | 1084 | 1099 | 16204 | 16219 | ATGCTGAGACGCAGGT | 0 | 1692 |
| 959320 | 1256 | 1271 | 19096 | 19111 | AATGGCAGATTCCACA | 25 | 1693 |
| 959330 | 1642 | 1657 | 25405 | 25420 | CTCTGAAAGAATCTGC | 75 | 1694 |
| 959340 | 1659 | 1674 | 25422 | 25437 | ATGGGAAACTTTAGCA | 77 | 1695 |
| 959350 | 1839 | 1854 | 25602 | 25617 | TGTCATTTCCCAACCA | 79 | 1696 |
| 959360 | 2114 | 2129 | 25877 | 25892 | TTTTAGTAGTCAAGGT | 88 | 1697 |
| 959370 | 2223 | 2238 | 25986 | 26001 | GCTTCCTTACATTTTT | 85 | 1698 |
| 959380 | 2269 | 2284 | 26032 | 26047 | TAACATCTCACTGATT | 42 | 1699 |
| 959390 | N/A | N/A | 4311 | 4326 | CTAGTGAGAAACAAAC | 0 | 1700 |
| 959400 | N/A | N/A | 4761 | 4776 | TTATTGTTGCTAAACC | 32 | 1701 |
| 959410 | N/A | N/A | 4863 | 4878 | ACTTTAGGCTCCTGGG | 60 | 1702 |
| 959420 | N/A | N/A | 5285 | 5300 | AGCCATAAATCTTGGG | 24 | 1703 |
| 959430 | N/A | N/A | 5573 | 5588 | ATGACATCATGGCTTC | 93 | 1704 |
| 959440 | N/A | N/A | 5603 | 5618 | TTATTCAATGTGGCTT | 95 | 1705 |
| 959450 | N/A | N/A | 5640 | 5655 | GGGCTCAATGAAATTA | 12 | 1706 |
| 959460 | N/A | N/A | 5713 | 5728 | CTTAGTATGAAATATC | 86 | 1707 |
| 959470 | N/A | N/A | 5808 | 5823 | TACTGTCTACTATGGG | 91 | 1708 |
| 959480 | N/A | N/A | 6157 | 6172 | CTTACATCCACGACTT | 35 | 1709 |
| 959660 | N/A | N/A | 12153 | 12168 | CAGTAACTGGTAGCTC | 74 | 1710 |
| 959670 | N/A | N/A | 12169 | 12184 | TGTTTGATTGTGCAGA | 95 | 1711 |
| 959680 | N/A | N/A | 12210 | 12225 | CGCCTTTTATTTCCGT | 92 | 1712 |
| 959690 | N/A | N/A | 12313 | 12328 | ATAACCACAGCCTGGG | 66 | 1713 |
| 959700 | N/A | N/A | 12675 | 12690 | ATAAGAATCATCTTAG | 7 | 1714 |
| 959710 | N/A | N/A | 12711 | 12726 | ACTACCGAACGCAGTT | 41 | 1715 |
| 959720 | N/A | N/A | 12757 | 12772 | TAGAGTGGTAAGGCAT | 84 | 1716 |
| 959730 | N/A | N/A | 12793 | 12808 | GGTTGGTGTACATGGT | 96 | 1717 |
| 959740 | N/A | N/A | 12880 | 12895 | TCCTGTTAGACAGCTT | 93 | 1718 |
| 959750 | N/A | N/A | 12902 | 12917 | ATAAAGCACACGGGAA | 86 | 1719 |
| 959760 | N/A | N/A | 12931 | 12946 | TAAGAGCTGTCTCCTC | 85 | 1720 |
| 959770 | N/A | N/A | 12972 | 12987 | CTAACAAACTTTGCAG | 79 | 1721 |
| 959780 | N/A | N/A | 13392 | 13407 | TGTCACCCTTCCACGG | 15 | 1722 |
| 959790 | N/A | N/A | 13526 | 13541 | ATTGGAAGACCGCAGA | 43 | 1723 |
| 959800 | N/A | N/A | 13706 | 13721 | CCGCTAGTAAATGCTT | 44 | 1724 |
| 959810 | N/A | N/A | 13737 | 13752 | AACTAAGGCAAATCTC | 77 | 1725 |
| 959820 | N/A | N/A | 13915 | 13930 | GAGTCATGACATCCCA | 89 | 1726 |
| 959830 | N/A | N/A | 14299 | 14314 | GCAGATAAATACACAT | 93 | 1727 |
| 959840 | N/A | N/A | 14424 | 14439 | TTTCCCATCGACACAG | 78 | 1728 |
| 959850 | N/A | N/A | 14571 | 14586 | GCTCCTACTTATCCCC | 76 | 1729 |
| 959860 | N/A | N/A | 15202 | 15217 | TATTGCCAGGTATCTG | 64 | 1730 |
| 959870 | N/A | N/A | 15599 | 15614 | CAATACATAGCAGAGC | 23 | 1731 |
| 959880 | N/A | N/A | 17192 | 17207 | TTAGTCCGTGTTCAGG | 90 | 1732 |
| 959890 | N/A | N/A | 17222 | 17237 | GTAGCTGGTTTGTGGG | 20 | 1733 |
| 959900 | N/A | N/A | 17295 | 17310 | CATCTCTTAGGGCACC | 79 | 1734 |
| 959910 | N/A | N/A | 18393 | 18408 | GTTTGGAAGTCGCCAT | 77 | 1735 |
| 959920 | N/A | N/A | 20284 | 20299 | AGGAAGCCCAATCAAG | 85 | 1736 |
| 959930 | N/A | N/A | 20512 | 20527 | CAGATTGAGTCTCCTG | 10 | 1737 |
| 959940 | N/A | N/A | 20607 | 20622 | TTTTACGATCATCATT | 72 | 1738 |
| 959950 | N/A | N/A | 20661 | 20676 | GCTTAGAAATTGCAGT | 75 | 1739 |
| 959960 | N/A | N/A | 20812 | 20827 | AGGGTAATATTCAGAC | 86 | 1740 |
| 959970 | N/A | N/A | 20934 | 20949 | TGTAGCAGACAGATCA | 74 | 1741 |
| 959980 | N/A | N/A | 21000 | 21015 | TTTAACAGCTCAGGTA | 66 | 1742 |
| 959990 | N/A | N/A | 21405 | 21420 | ATTCTAGACATGGCCA | 51 | 1743 |
| 960000 | N/A | N/A | 21442 | 21457 | AACCAATATACTGAGA | 71 | 1744 |
| 960010 | N/A | N/A | 21545 | 21560 | AGACATATGACATTTC | 91 | 1745 |
| 960020 | N/A | N/A | 22765 | 22780 | ACATGACAGACTAACT | 55 | 1746 |
| 960030 | N/A | N/A | 24039 | 24054 | CATCAATGCTGCACTC | 13 | 1747 |

TABLE 28
Inhibition of PNPLA3 mRNA by 3-10-3 cEt gapmers targeting SEQ ID NO: 1 and 2

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | PNPLA3 | SEQ |
| Compound | Start | Stop | Start | Stop | Sequence | % | ID |
| Number | Site | Site | Site | Site | (5′ to 3′) | Inhibition | NO |
| 915609 | 705 | 720 | 11923 | 11938 | TGATGGTTGTTTTGGC | 98 | 702 |
| 959271 | 425 | 440 | 5999 | 6014 | GTTGAAGGATGGATGG | 90 | 1748 |
| 959281 | 511 | 526 | 6085 | 6100 | AGAGAGATGCCTATTT | 73 | 1749 |
| 959291 | 813 | 828 | 12031 | 12046 | GGTTCCCTGTGCAGAG | 79 | 1750 |
| 959301 | 904 | 919 | 13640 | 13655 | AAGAACCTGAATGCAT | 48 | 1751 |
| 959311 | 1085 | 1100 | 16205 | 16220 | GATGCTGAGACGCAGG | 0 | 1752 |
| 959321 | 1602 | 1617 | 25365 | 25380 | ACAGACTCTTCTCTAG | 45 | 1753 |
| 959331 | 1643 | 1658 | 25406 | 25421 | CCTCTGAAAGAATCTG | 81 | 1754 |
| 959341 | 1673 | 1688 | 25436 | 25451 | GTAGCTGCACAAAGAT | 69 | 1755 |
| 959351 | 1842 | 1857 | 25605 | 25620 | TGGTGTCATTTCCCAA | 19 | 1756 |
| 959361 | 2115 | 2130 | 25878 | 25893 | TTTTTAGTAGTCAAGG | 91 | 1757 |
| 959371 | 2240 | 2255 | 26003 | 26018 | ATTCAACAGGTAACAA | 69 | 1758 |
| 959381 | 2271 | 2286 | 26034 | 26049 | ACTAACATCTCACTGA | 33 | 1759 |
| 959391 | N/A | N/A | 4313 | 4328 | AGCTAGTGAGAAACAA | 47 | 1760 |
| 959401 | N/A | N/A | 4764 | 4779 | CTTTTATTGTTGCTAA | 77 | 1761 |
| 959411 | N/A | N/A | 4868 | 4883 | AGTGTACTTTAGGCTC | 90 | 1762 |
| 959421 | N/A | N/A | 5286 | 5301 | CAGCCATAAATCTTGG | 0 | 1763 |
| 959431 | N/A | N/A | 5574 | 5589 | AATGACATCATGGCTT | 74 | 1764 |
| 959441 | N/A | N/A | 5604 | 5619 | TTTATTCAATGTGGCT | 96 | 1765 |
| 959451 | N/A | N/A | 5642 | 5657 | TTGGGCTCAATGAAAT | 0 | 1766 |
| 959461 | N/A | N/A | 5714 | 5729 | GCTTAGTATGAAATAT | 78 | 1767 |
| 959471 | N/A | N/A | 5809 | 5824 | GTACTGTCTACTATGG | 78 | 1768 |
| 959481 | N/A | N/A | 6160 | 6175 | CTGCTTACATCCACGA | 4 | 1769 |
| 959661 | N/A | N/A | 12154 | 12169 | ACAGTAACTGGTAGCT | 72 | 1770 |
| 959671 | N/A | N/A | 12170 | 12185 | CTGTTTGATTGTGCAG | 50 | 1771 |
| 959681 | N/A | N/A | 12211 | 12226 | GCGCCTTTTATTTCCG | 14 | 1772 |
| 959691 | N/A | N/A | 12322 | 12337 | CCTGACTATATAACCA | 56 | 1773 |
| 959701 | N/A | N/A | 12689 | 12704 | GACCGTGTTTCCAAAT | 97 | 1774 |
| 959711 | N/A | N/A | 12712 | 12727 | AACTACCGAACGCAGT | 48 | 1775 |
| 959721 | N/A | N/A | 12759 | 12774 | GGTAGAGTGGTAAGGC | 95 | 1776 |
| 959731 | N/A | N/A | 12828 | 12843 | CATAGCCTTCTTTCTT | 93 | 1777 |
| 959741 | N/A | N/A | 12882 | 12897 | AATCCTGTTAGACAGC | 90 | 1778 |
| 959751 | N/A | N/A | 12903 | 12918 | TATAAAGCACACGGGA | 82 | 1779 |
| 959761 | N/A | N/A | 12933 | 12948 | AATAAGAGCTGTCTCC | 87 | 1780 |
| 959771 | N/A | N/A | 12974 | 12989 | CCCTAACAAACTTTGC | 62 | 1781 |
| 959781 | N/A | N/A | 13394 | 13409 | AATGTCACCCTTCCAC | 90 | 1782 |
| 959791 | N/A | N/A | 13527 | 13542 | CATTGGAAGACCGCAG | 76 | 1783 |
| 959801 | N/A | N/A | 13707 | 13722 | ACCGCTAGTAAATGCT | 35 | 1784 |
| 959811 | N/A | N/A | 13740 | 13755 | TAGAACTAAGGCAAAT | 65 | 1785 |
| 959821 | N/A | N/A | 13916 | 13931 | GGAGTCATGACATCCC | 42 | 1786 |
| 959831 | N/A | N/A | 14302 | 14317 | TGAGCAGATAAATACA | 75 | 1787 |
| 959841 | N/A | N/A | 14425 | 14440 | CTTTCCCATCGACACA | 79 | 1788 |
| 959851 | N/A | N/A | 14573 | 14588 | GCGCTCCTACTTATCC | 0 | 1789 |
| 959861 | N/A | N/A | 15203 | 15218 | ATATTGCCAGGTATCT | 67 | 1790 |
| 959871 | N/A | N/A | 15763 | 15778 | GTGTTGGTTTATAACA | 13 | 1791 |
| 959881 | N/A | N/A | 17194 | 17209 | ACTTAGTCCGTGTTCA | 45 | 1792 |
| 959891 | N/A | N/A | 17224 | 17239 | CTGTAGCTGGTTTGTG | 42 | 1793 |
| 959901 | N/A | N/A | 17296 | 17311 | CCATCTCTTAGGGCAC | 53 | 1794 |
| 959911 | N/A | N/A | 18394 | 18409 | TGTTTGGAAGTCGCCA | 87 | 1795 |
| 959921 | N/A | N/A | 20289 | 20304 | ATCAGAGGAAGCCCAA | 84 | 1796 |
| 959931 | N/A | N/A | 20514 | 20529 | ACCAGATTGAGTCTCC | 91 | 1797 |
| 959941 | N/A | N/A | 20613 | 20628 | CTCTAATTTTACGATC | 70 | 1798 |
| 959951 | N/A | N/A | 20662 | 20677 | AGCTTAGAAATTGCAG | 0 | 1799 |
| 959961 | N/A | N/A | 20813 | 20828 | CAGGGTAATATTCAGA | 87 | 1800 |
| 959971 | N/A | N/A | 20936 | 20951 | TTTGTAGCAGACAGAT | 18 | 1801 |
| 959981 | N/A | N/A | 21001 | 21016 | TTTTAACAGCTCAGGT | 71 | 1802 |
| 959991 | N/A | N/A | 21406 | 21421 | AATTCTAGACATGGCC | 25 | 1803 |
| 960001 | N/A | N/A | 21443 | 21458 | TAACCAATATACTGAG | 72 | 1804 |
| 960011 | N/A | N/A | 22023 | 22038 | CGCAAAAAGACAACGA | 16 | 1805 |
| 960021 | N/A | N/A | 22766 | 22781 | GACATGACAGACTAAC | 76 | 1806 |
| 960031 | N/A | N/A | 24040 | 24055 | CCATCAATGCTGCACT | 61 | 1807 |

TABLE 29
Inhibition of PNPLA3 mRNA by 3-10-3 cEt gapmers targeting SEQ ID NO: 1 and 2

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | PNPLA3 | SEQ |
| Compound | Start | Stop | Start | Stop | Sequence | % | ID |
| Number | Site | Site | Site | Site | (5′ to 3′) | Inhibition | NO |
| 915609 | 705 | 720 | 11923 | 11938 | TGATGGTTGTTTTGGC | 97 | 702 |
| 959272 | 426 | 441 | 6000 | 6015 | AGTTGAAGGATGGATG | 76 | 1808 |
| 959282 | 517 | 532 | 6091 | 6106 | CTGGTAAGAGAGATGC | 64 | 1809 |
| 959292 | 815 | 830 | 12033 | 12048 | GAGGTTCCCTGTGCAG | 81 | 1810 |
| 959302 | 905 | 920 | 13641 | 13656 | CAAGAACCTGAATGCA | 65 | 1811 |
| 959312 | 1087 | 1102 | 16207 | 16222 | AGGATGCTGAGACGCA | 66 | 1812 |
| 959322 | 1604 | 1619 | 25367 | 25382 | TCACAGACTCTTCTCT | 50 | 1813 |
| 959332 | 1644 | 1659 | 25407 | 25422 | ACCTCTGAAAGAATCT | 69 | 1814 |
| 959342 | 1675 | 1690 | 25438 | 25453 | AGGTAGCTGCACAAAG | 78 | 1815 |
| 959352 | 1903 | 1918 | 25666 | 25681 | CAGTCTGACCATTAAT | 77 | 1816 |
| 959362 | 2173 | 2188 | 25936 | 25951 | GAAAAAGGTGTTCTAA | 85 | 1817 |
| 959372 | 2242 | 2257 | 26005 | 26020 | AAATTCAACAGGTAAC | 62 | 1818 |
| 959382 | 2275 | 2290 | 26038 | 26053 | TTCTACTAACATCTCA | 80 | 1819 |
| 959392 | N/A | N/A | 4732 | 4747 | ATGATCATGTGGCGGT | 80 | 1820 |
| 959402 | N/A | N/A | 4765 | 4780 | ACTTTTATTGTTGCTA | 86 | 1821 |
| 959412 | N/A | N/A | 4869 | 4884 | GAGTGTACTTTAGGCT | 94 | 1822 |
| 959422 | N/A | N/A | 5389 | 5404 | CTATCAGGTGCAGGAG | 93 | 1823 |
| 959432 | N/A | N/A | 5575 | 5590 | CAATGACATCATGGCT | 90 | 1824 |
| 959442 | N/A | N/A | 5607 | 5622 | TACTTTATTCAATGTG | 0 | 1825 |
| 959452 | N/A | N/A | 5643 | 5658 | ATTGGGCTCAATGAAA | 35 | 1826 |
| 959462 | N/A | N/A | 5716 | 5731 | TGGCTTAGTATGAAAT | 80 | 1827 |
| 959472 | N/A | N/A | 5864 | 5879 | TTTGGCAAGGCCAGAA | 0 | 1828 |
| 959482 | N/A | N/A | 6960 | 6975 | GCATAGAGGAAGCTCG | 32 | 1829 |
| 959662 | N/A | N/A | 12155 | 12170 | GACAGTAACTGGTAGC | 92 | 1830 |
| 959672 | N/A | N/A | 12172 | 12187 | TTCTGTTTGATTGTGC | 97 | 1831 |
| 959682 | N/A | N/A | 12280 | 12295 | AGGTCTGGGTATATGT | 93 | 1832 |
| 959692 | N/A | N/A | 12323 | 12338 | CCCTGACTATATAACC | 32 | 1833 |
| 959702 | N/A | N/A | 12691 | 12706 | TTGACCGTGTTTCCAA | 94 | 1834 |
| 959712 | N/A | N/A | 12713 | 12728 | AAACTACCGAACGCAG | 92 | 1835 |
| 959722 | N/A | N/A | 12760 | 12775 | TGGTAGAGTGGTAAGG | 94 | 1836 |
| 959732 | N/A | N/A | 12829 | 12844 | GCATAGCCTTCTTTCT | 87 | 1837 |
| 959742 | N/A | N/A | 12883 | 12898 | CAATCCTGTTAGACAG | 13 | 1838 |
| 959752 | N/A | N/A | 12904 | 12919 | TTATAAAGCACACGGG | 87 | 1839 |
| 959762 | N/A | N/A | 12934 | 12949 | CAATAAGAGCTGTCTC | 81 | 1840 |
| 959772 | N/A | N/A | 13370 | 13385 | TGCAGGCACCCCAGCA | 0 | 1841 |
| 959782 | N/A | N/A | 13395 | 13410 | GAATGTCACCCTTCCA | 90 | 1842 |
| 959792 | N/A | N/A | 13528 | 13543 | TCATTGGAAGACCGCA | 84 | 1843 |
| 959802 | N/A | N/A | 13708 | 13723 | GACCGCTAGTAAATGC | 57 | 1844 |
| 959812 | N/A | N/A | 13741 | 13756 | TTAGAACTAAGGCAAA | 78 | 1845 |
| 959822 | N/A | N/A | 13917 | 13932 | TGGAGTCATGACATCC | 0 | 1846 |
| 959832 | N/A | N/A | 14303 | 14318 | CTGAGCAGATAAATAC | 74 | 1847 |
| 959842 | N/A | N/A | 14553 | 14568 | AGTCTTAATGTGGATT | 76 | 1848 |
| 959852 | N/A | N/A | 14667 | 14682 | AGTGTCCCCATCCCCA | 54 | 1849 |
| 959862 | N/A | N/A | 15204 | 15219 | AATATTGCCAGGTATC | 56 | 1850 |
| 959872 | N/A | N/A | 15765 | 15780 | TAGTGTTGGTTTATAA | 89 | 1851 |
| 959882 | N/A | N/A | 17196 | 17211 | TGACTTAGTCCGTGTT | 43 | 1852 |
| 959892 | N/A | N/A | 17225 | 17240 | TCTGTAGCTGGTTTGT | 61 | 1853 |
| 959902 | N/A | N/A | 17298 | 17313 | TCCCATCTCTTAGGGC | 24 | 1854 |
| 959912 | N/A | N/A | 18396 | 18411 | TATGTTTGGAAGTCGC | 91 | 1855 |
| 959922 | N/A | N/A | 20290 | 20305 | AATCAGAGGAAGCCCA | 40 | 1856 |
| 959932 | N/A | N/A | 20515 | 20530 | AACCAGATTGAGTCTC | 72 | 1857 |
| 959942 | N/A | N/A | 20614 | 20629 | TCTCTAATTTTACGAT | 23 | 1858 |
| 959952 | N/A | N/A | 20663 | 20678 | CAGCTTAGAAATTGCA | 24 | 1859 |
| 959962 | N/A | N/A | 20814 | 20829 | CCAGGGTAATATTCAG | 87 | 1860 |
| 959972 | N/A | N/A | 20937 | 20952 | CTTTGTAGCAGACAGA | 50 | 1861 |
| 959982 | N/A | N/A | 21003 | 21018 | TATTTTAACAGCTCAG | 94 | 1862 |
| 959992 | N/A | N/A | 21409 | 21424 | TGCAATTCTAGACATG | 12 | 1863 |
| 960002 | N/A | N/A | 21445 | 21460 | ACTAACCAATATACTG | 55 | 1864 |
| 960012 | N/A | N/A | 22541 | 22556 | CAACAGATTACTGGAC | 28 | 1865 |
| 960022 | N/A | N/A | 22768 | 22783 | AGGACATGACAGACTA | 66 | 1866 |
| 960032 | N/A | N/A | 24041 | 24056 | ACCATCAATGCTGCAC | 79 | 1867 |

TABLE 30
Inhibition of PNPLA3 mRNA by 3-10-3 cEt gapmers targeting SEQ ID NO: 1 and 2

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | PNPLA3 | SEQ |
| Compound | Start | Stop | Start | Stop | Sequence | % | ID |
| Number | Site | Site | Site | Site | (5′ to 3′) | Inhibition | NO |
| 915609 | 705 | 720 | 11923 | 11938 | TGATGGTTGTTTTGGC | 98 | 702 |
| 959273 | 427 | 442 | 6001 | 6016 | AAGTTGAAGGATGGAT | 86 | 1868 |
| 959283 | 694 | 709 | 11912 | 11927 | TTGGCATCAATGAAGG | 73 | 1869 |
| 959293 | 816 | 831 | 12034 | 12049 | AGAGGTTCCCTGTGCA | 80 | 1870 |
| 959303 | 906 | 921 | 13642 | 13657 | CCAAGAACCTGAATGC | 65 | 1871 |
| 959313 | 1090 | 1105 | 16210 | 16225 | GGCAGGATGCTGAGAC | 43 | 1872 |
| 959323 | 1605 | 1620 | 25368 | 25383 | CTCACAGACTCTTCTC | 81 | 1873 |
| 959333 | 1646 | 1661 | 25409 | 25424 | GCACCTCTGAAAGAAT | 51 | 1874 |
| 959343 | 1677 | 1692 | 25440 | 25455 | GGAGGTAGCTGCACAA | 73 | 1875 |
| 959353 | 1904 | 1919 | 25667 | 25682 | ACAGTCTGACCATTAA | 85 | 1876 |
| 959363 | 2179 | 2194 | 25942 | 25957 | TTAGGTGAAAAAGGTG | 91 | 1877 |
| 959373 | 2258 | 2273 | 26021 | 26036 | TGATTCACATAATACA | 71 | 1878 |
| 959383 | 2277 | 2292 | 26040 | 26055 | TATTCTACTAACATCT | 38 | 1879 |
| 959393 | N/A | N/A | 4733 | 4748 | AATGATCATGTGGCGG | 93 | 1880 |
| 959403 | N/A | N/A | 4767 | 4782 | TGACTTTTATTGTTGC | 87 | 1881 |
| 959413 | N/A | N/A | 4870 | 4885 | TGAGTGTACTTTAGGC | 94 | 1882 |
| 959423 | N/A | N/A | 5392 | 5407 | ATGCTATCAGGTGCAG | 0 | 1883 |
| 959433 | N/A | N/A | 5578 | 5593 | GCACAATGACATCATG | 86 | 1884 |
| 959443 | N/A | N/A | 5608 | 5623 | TTACTTTATTCAATGT | 9 | 1885 |
| 959453 | N/A | N/A | 5644 | 5659 | TATTGGGCTCAATGAA | 80 | 1886 |
| 959463 | N/A | N/A | 5798 | 5813 | TATGGGAGCCACATGT | 4 | 1887 |
| 959473 | N/A | N/A | 5866 | 5881 | CTTTTGGCAAGGCCAG | 0 | 1888 |
| 959483 | N/A | N/A | 7199 | 7214 | TTAAACAGAGGATGCA | 31 | 1889 |
| 959663 | N/A | N/A | 12156 | 12171 | AGACAGTAACTGGTAG | 75 | 1890 |
| 959673 | N/A | N/A | 12199 | 12214 | TCCGTTAACCATCAAG | 95 | 1891 |
| 959683 | N/A | N/A | 12282 | 12297 | TTAGGTCTGGGTATAT | 94 | 1892 |
| 959693 | N/A | N/A | 12324 | 12339 | CCCCTGACTATATAAC | 0 | 1893 |
| 959703 | N/A | N/A | 12692 | 12707 | CTTGACCGTGTTTCCA | 97 | 1894 |
| 959713 | N/A | N/A | 12715 | 12730 | TTAAACTACCGAACGC | 95 | 1895 |
| 959723 | N/A | N/A | 12761 | 12776 | ATGGTAGAGTGGTAAG | 77 | 1896 |
| 959733 | N/A | N/A | 12832 | 12847 | ATTGCATAGCCTTCTT | 95 | 1897 |
| 959743 | N/A | N/A | 12884 | 12899 | CCAATCCTGTTAGACA | 83 | 1898 |
| 959753 | N/A | N/A | 12908 | 12923 | CTGCTTATAAAGCACA | 2 | 1899 |
| 959763 | N/A | N/A | 12935 | 12950 | ACAATAAGAGCTGTCT | 85 | 1900 |
| 959773 | N/A | N/A | 13372 | 13387 | TTTGCAGGCACCCCAG | 63 | 1901 |
| 959783 | N/A | N/A | 13396 | 13411 | TGAATGTCACCCTTCC | 53 | 1902 |
| 959793 | N/A | N/A | 13531 | 13546 | GCATCATTGGAAGACC | 86 | 1903 |
| 959803 | N/A | N/A | 13713 | 13728 | ACCAAGACCGCTAGTA | 38 | 1904 |
| 959813 | N/A | N/A | 13743 | 13758 | TGTTAGAACTAAGGCA | 79 | 1905 |
| 959823 | N/A | N/A | 13919 | 13934 | CCTGGAGTCATGACAT | 7 | 1906 |
| 959833 | N/A | N/A | 14304 | 14319 | TCTGAGCAGATAAATA | 39 | 1907 |
| 959843 | N/A | N/A | 14554 | 14569 | AAGTCTTAATGTGGAT | 84 | 1908 |
| 959853 | N/A | N/A | 14669 | 14684 | TTAGTGTCCCCATCCC | 78 | 1909 |
| 959863 | N/A | N/A | 15205 | 15220 | GAATATTGCCAGGTAT | 86 | 1910 |
| 959873 | N/A | N/A | 15766 | 15781 | TTAGTGTTGGTTTATA | 92 | 1911 |
| 959883 | N/A | N/A | 17198 | 17213 | TTTGACTTAGTCCGTG | 86 | 1912 |
| 959893 | N/A | N/A | 17226 | 17241 | CTCTGTAGCTGGTTTG | 82 | 1913 |
| 959903 | N/A | N/A | 17601 | 17616 | TTGATAGTGAATGTGT | 83 | 1914 |
| 959913 | N/A | N/A | 18397 | 18412 | ATATGTTTGGAAGTCG | 91 | 1915 |
| 959923 | N/A | N/A | 20291 | 20306 | CAATCAGAGGAAGCCC | 34 | 1916 |
| 959933 | N/A | N/A | 20516 | 20531 | TAACCAGATTGAGTCT | 66 | 1917 |
| 959943 | N/A | N/A | 20615 | 20630 | GTCTCTAATTTTACGA | 53 | 1918 |
| 959953 | N/A | N/A | 20664 | 20679 | ACAGCTTAGAAATTGC | 89 | 1919 |
| 959963 | N/A | N/A | 20843 | 20858 | CTGTATTAGCTCAATA | 67 | 1920 |
| 959973 | N/A | N/A | 20938 | 20953 | TCTTTGTAGCAGACAG | 84 | 1921 |
| 959983 | N/A | N/A | 21004 | 21019 | TTATTTTAACAGCTCA | 92 | 1922 |
| 959993 | N/A | N/A | 21410 | 21425 | CTGCAATTCTAGACAT | 29 | 1923 |
| 960003 | N/A | N/A | 21535 | 21550 | CATTTCAGAGTATAAG | 46 | 1924 |
| 960013 | N/A | N/A | 22708 | 22723 | GATGTGAGTGAAATAA | 69 | 1925 |
| 960023 | N/A | N/A | 22769 | 22784 | AAGGACATGACAGACT | 68 | 1926 |
| 960033 | N/A | N/A | 24043 | 24058 | CCACCATCAATGCTGC | 58 | 1927 |

TABLE 31
Inhibition of PNPLA3 mRNA by 3-10-3 cEt gapmers targeting SEQ ID NO: 1 and 2

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | PNPLA3 | SEQ |
| Compound | Start | Stop | Start | Stop | Sequence | % | ID |
| Number | Site | Site | Site | Site | (5′ to 3′) | Inhibition | NO |
| 915609 | 705 | 720 | 11923 | 11938 | TGATGGTTGTTTTGGC | 99 | 702 |
| 959284 | 695 | 710 | 11913 | 11928 | TTTGGCATCAATGAAG | 69 | 1928 |
| 959294 | 817 | 832 | 12035 | 12050 | TAGAGGTTCCCTGTGC | 83 | 1929 |
| 959314 | 1091 | 1106 | 16211 | 16226 | GGGCAGGATGCTGAGA | 22 | 1930 |
| 959324 | 1606 | 1621 | 25369 | 25384 | ACTCACAGACTCTTCT | 68 | 1931 |
| 959334 | 1647 | 1662 | 25410 | 25425 | AGCACCTCTGAAAGAA | 67 | 1932 |
| 959344 | 1678 | 1693 | 25441 | 25456 | CGGAGGTAGCTGCACA | 85 | 1933 |
| 959354 | 1926 | 1941 | 25689 | 25704 | ATTCTAAGAACCTCAT | 51 | 1934 |
| 959384 | N/A | N/A | 4303 | 4318 | AAACAAACCCTCCGTC | 10 | 1935 |
| 959394 | N/A | N/A | 4734 | 4749 | AAATGATCATGTGGCG | 94 | 1936 |
| 959404 | N/A | N/A | 4768 | 4783 | CTGACTTTTATTGTTG | 74 | 1937 |
| 959414 | N/A | N/A | 4871 | 4886 | GTGAGTGTACTTTAGG | 98 | 1938 |
| 959424 | N/A | N/A | 5393 | 5408 | AATGCTATCAGGTGCA | 31 | 1939 |
| 959434 | N/A | N/A | 5579 | 5594 | TGCACAATGACATCAT | 88 | 1940 |
| 959444 | N/A | N/A | 5621 | 5636 | CTACCTGTGTCTTTTA | 90 | 1941 |
| 959454 | N/A | N/A | 5647 | 5662 | ATATATTGGGCTCAAT | 81 | 1942 |
| 959474 | N/A | N/A | 5867 | 5882 | ACTTTTGGCAAGGCCA | 17 | 1943 |
| 959484 | N/A | N/A | 7211 | 7226 | CCGCAAACAAGGTTAA | 22 | 1944 |
| 959684 | N/A | N/A | 12283 | 12298 | TTTAGGTCTGGGTATA | 93 | 1945 |
| 959694 | N/A | N/A | 12325 | 12340 | CCCCCTGACTATATAA | 18 | 1946 |
| 959704 | N/A | N/A | 12693 | 12708 | TCTTGACCGTGTTTCC | 97 | 1947 |
| 959734 | N/A | N/A | 12834 | 12849 | GCATTGCATAGCCTTC | 96 | 1948 |
| 959744 | N/A | N/A | 12886 | 12901 | AACCAATCCTGTTAGA | 59 | 1949 |
| 959754 | N/A | N/A | 12909 | 12924 | TCTGCTTATAAAGCAC | 100 | 1950 |
| 959774 | N/A | N/A | 13373 | 13388 | CTTTGCAGGCACCCCA | 72 | 1951 |
| 959784 | N/A | N/A | 13398 | 13413 | CTTGAATGTCACCCTT | 92 | 1952 |
| 959804 | N/A | N/A | 13715 | 13730 | TTACCAAGACCGCTAG | 47 | 1953 |
| 959824 | N/A | N/A | 14228 | 14243 | ACTTTTAGTATTAAAG | 0 | 1954 |
| 959844 | N/A | N/A | 14555 | 14570 | AAAGTCTTAATGTGGA | 88 | 1955 |
| 959854 | N/A | N/A | 14670 | 14685 | CTTAGTGTCCCCATCC | 76 | 1956 |
| 959874 | N/A | N/A | 15767 | 15782 | GTTAGTGTTGGTTTAT | 95 | 1957 |
| 959884 | N/A | N/A | 17199 | 17214 | GTTTGACTTAGTCCGT | 96 | 1958 |
| 959914 | N/A | N/A | 18398 | 18413 | AATATGTTTGGAAGTC | 87 | 1959 |
| 959934 | N/A | N/A | 20518 | 20533 | AGTAACCAGATTGAGT | 96 | 1960 |
| 959954 | N/A | N/A | 20665 | 20680 | CACAGCTTAGAAATTG | 88 | 1961 |
| 959964 | N/A | N/A | 20844 | 20859 | CCTGTATTAGCTCAAT | 92 | 1962 |
| 959974 | N/A | N/A | 20940 | 20955 | CCTCTTTGTAGCAGAC | 84 | 1963 |
| 959984 | N/A | N/A | 21006 | 21021 | GGTTATTTTAACAGCT | 85 | 1964 |
| 959994 | N/A | N/A | 21412 | 21427 | ACCTGCAATTCTAGAC | 61 | 1965 |
| 960014 | N/A | N/A | 22710 | 22725 | AAGATGTGAGTGAAAT | 70 | 1966 |
| 960024 | N/A | N/A | 22770 | 22785 | AAAGGACATGACAGAC | 87 | 1967 |

TABLE 32
Inhibition of PNPLA3 mRNA by 3-10-3 cEt gapmers targeting SEQ ID NO: 1 and 2

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | PNPLA3 | SEQ |
| Compound | Start | Stop | Start | Stop | Sequence | % | ID |
| Number | Site | Site | Site | Site | (5′ to 3′) | Inhibition | NO |
| 959275 | 440 | 455 | 6014 | 6029 | GAGGAACTTGCTTAAG | 81 | 1968 |
| 959285 | 696 | 711 | 11914 | 11929 | TTTTGGCATCAATGAA | 62 | 1969 |
| 959305 | 1066 | 1081 | 16186 | 16201 | TCTAGCAGCTCATCTC | 64 | 1970 |
| 959335 | 1649 | 1664 | 25412 | 25427 | TTAGCACCTCTGAAAG | 72 | 1971 |
| 959345 | 1804 | 1819 | 25567 | 25582 | ATGTATTAGAGTTAAG | 77 | 1972 |
| 959355 | 1927 | 1942 | 25690 | 25705 | CATTCTAAGAACCTCA | 71 | 1973 |
| 959365 | 2183 | 2198 | 25946 | 25961 | TTAGTTAGGTGAAAAA | 81 | 1974 |
| 959375 | 2261 | 2276 | 26024 | 26039 | CACTGATTCACATAAT | 70 | 1975 |
| 959395 | N/A | N/A | 4737 | 4752 | TGCAAATGATCATGTG | 66 | 1976 |
| 959405 | N/A | N/A | 4769 | 4784 | GCTGACTTTTATTGTT | 84 | 1977 |
| 959415 | N/A | N/A | 4872 | 4887 | AGTGAGTGTACTTTAG | 94 | 1978 |
| 959425 | N/A | N/A | 5395 | 5410 | TTAATGCTATCAGGTG | 81 | 1979 |
| 959435 | N/A | N/A | 5580 | 5595 | ATGCACAATGACATCA | 86 | 1980 |
| 959445 | N/A | N/A | 5624 | 5639 | ATTCTACCTGTGTCTT | 97 | 1981 |
| 959455 | N/A | N/A | 5651 | 5666 | TTGGATATATTGGGCT | 97 | 1982 |
| 959475 | N/A | N/A | 5868 | 5883 | TACTTTTGGCAAGGCC | 70 | 1983 |
| 959485 | N/A | N/A | 7697 | 7712 | GCACAGAGTAGGTTAA | 72 | 1984 |
| 959655 | N/A | N/A | 12146 | 12161 | TGGTAGCTCCTGGCAA | 55 | 1985 |
| 959675 | N/A | N/A | 12201 | 12216 | TTTCCGTTAACCATCA | 94 | 1986 |
| 959695 | N/A | N/A | 12667 | 12682 | CATCTTAGTGGCTGGG | 93 | 1987 |
| 959705 | N/A | N/A | 12695 | 12710 | GTTCTTGACCGTGTTT | 97 | 1988 |
| 959715 | N/A | N/A | 12717 | 12732 | GGTTAAACTACCGAAC | 11 | 1989 |
| 959725 | N/A | N/A | 12783 | 12798 | CATGGTCTGCAAATTT | 89 | 1990 |
| 959745 | N/A | N/A | 12887 | 12902 | AAACCAATCCTGTTAG | 46 | 1991 |
| 959755 | N/A | N/A | 12910 | 12925 | ATCTGCTTATAAAGCA | 42 | 1992 |
| 959775 | N/A | N/A | 13374 | 13389 | ACTTTGCAGGCACCCC | 87 | 1993 |
| 959785 | N/A | N/A | 13399 | 13414 | GCTTGAATGTCACCCT | 94 | 1994 |
| 959805 | N/A | N/A | 13716 | 13731 | TTTACCAAGACCGCTA | 86 | 1995 |
| 959825 | N/A | N/A | 14230 | 14245 | CAACTTTTAGTATTAA | 0 | 1996 |
| 959835 | N/A | N/A | 14417 | 14432 | TCGACACAGCATCACC | 62 | 1997 |
| 959855 | N/A | N/A | 14671 | 14686 | TCTTAGTGTCCCCATC | 78 | 1998 |
| 959865 | N/A | N/A | 15209 | 15224 | TTAGGAATATTGCCAG | 93 | 1999 |
| 959875 | N/A | N/A | 15769 | 15784 | GGGTTAGTGTTGGTTT | 94 | 2000 |
| 959895 | N/A | N/A | 17288 | 17303 | TAGGGCACCTCAAGAA | 0 | 2001 |
| 959915 | N/A | N/A | 18400 | 18415 | CAAATATGTTTGGAAG | 50 | 2002 |
| 959925 | N/A | N/A | 20293 | 20308 | CCCAATCAGAGGAAGC | 41 | 2003 |
| 959935 | N/A | N/A | 20599 | 20614 | TCATCATTATTACCTG | 91 | 2004 |
| 959955 | N/A | N/A | 20803 | 20818 | TTCAGACCAGGGTAAT | 91 | 2005 |
| 959965 | N/A | N/A | 20845 | 20860 | GCCTGTATTAGCTCAA | 90 | 2006 |
| 959975 | N/A | N/A | 20941 | 20956 | GCCTCTTTGTAGCAGA | 0 | 2007 |
| 959995 | N/A | N/A | 21434 | 21449 | TACTGAGAGGAAATGA | 64 | 2008 |
| 960015 | N/A | N/A | 22714 | 22729 | TGTAAAGATGTGAGTG | 78 | 2009 |
| 960025 | N/A | N/A | 22772 | 22787 | TCAAAGGACATGACAG | 80 | 2010 |
| 960037 | N/A | N/A | 22590 | 22605 | GAGCAACGAGGAAGGA | 68 | 2011 |

TABLE 33
Inhibition of PNPLA3 mRNA by 3-10-3 cEt gapmers targeting SEQ ID NO: 1 and 2

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | PNPLA3 | SEQ |
| Compound | Start | Stop | Start | Stop | Sequence | % | ID |
| Number | Site | Site | Site | Site | (5′ to 3′) | Inhibition | NO |
| 915609 | 705 | 720 | 11923 | 11938 | TGATGGTTGTTTTGGC | 98 | 702 |
| 959276 | 447 | 462 | 6021 | 6036 | CCTGTCGGAGGAACTT | 37 | 2012 |
| 959286 | 699 | 714 | 11917 | 11932 | TTGTTTTGGCATCAAT | 73 | 2013 |
| 959296 | 895 | 910 | 13631 | 13646 | AATGCATCCAAATATC | 0 | 2014 |
| 959306 | 1069 | 1084 | 16189 | 16204 | TGGTCTAGCAGCTCAT | 25 | 2015 |
| 959316 | 1221 | 1236 | 19061 | 19076 | TTACATAAGACATTAT | 35 | 2016 |
| 959326 | 1614 | 1629 | 25377 | 25392 | CTCAAGTGACTCACAG | 85 | 2017 |
| 959336 | 1650 | 1665 | 25413 | 25428 | TTTAGCACCTCTGAAA | 24 | 2018 |
| 959346 | 1834 | 1849 | 25597 | 25612 | TTTCCCAACCAGCTGA | 60 | 2019 |
| 959356 | 2096 | 2111 | 25859 | 25874 | TCATCTTTGCAGACCA | 90 | 2020 |
| 959366 | 2184 | 2199 | 25947 | 25962 | TTTAGTTAGGTGAAAA | 57 | 2021 |
| 959376 | 2262 | 2277 | 26025 | 26040 | TCACTGATTCACATAA | 86 | 2022 |
| 959386 | N/A | N/A | 4306 | 4321 | GAGAAACAAACCCTCC | 0 | 2023 |
| 959396 | N/A | N/A | 4738 | 4753 | GTGCAAATGATCATGT | 69 | 2024 |
| 959406 | N/A | N/A | 4771 | 4786 | AAGCTGACTTTTATTG | 57 | 2025 |
| 959416 | N/A | N/A | 5276 | 5291 | TCTTGGGATGCACAGG | 49 | 2026 |
| 959426 | N/A | N/A | 5397 | 5412 | CCTTAATGCTATCAGG | 0 | 2027 |
| 959436 | N/A | N/A | 5581 | 5596 | AATGCACAATGACATC | 69 | 2028 |
| 959446 | N/A | N/A | 5625 | 5640 | AATTCTACCTGTGTCT | 82 | 2029 |
| 959456 | N/A | N/A | 5652 | 5667 | TTTGGATATATTGGGC | 95 | 2030 |
| 959466 | N/A | N/A | 5802 | 5817 | CTACTATGGGAGCCAC | 64 | 2031 |
| 959476 | N/A | N/A | 5871 | 5886 | TAATACTTTTGGCAAG | 29 | 2032 |
| 959486 | N/A | N/A | 7784 | 7799 | TTATAGGCGAGAGCAC | 0 | 2033 |
| 959656 | N/A | N/A | 12147 | 12162 | CTGGTAGCTCCTGGCA | 44 | 2034 |
| 959666 | N/A | N/A | 12164 | 12179 | GATTGTGCAGACAGTA | 95 | 2035 |
| 959676 | N/A | N/A | 12202 | 12217 | ATTTCCGTTAACCATC | 93 | 2036 |
| 959686 | N/A | N/A | 12288 | 12303 | TGAGTTTTAGGTCTGG | 96 | 2037 |
| 959696 | N/A | N/A | 12669 | 12684 | ATCATCTTAGTGGCTG | 91 | 2038 |
| 959706 | N/A | N/A | 12696 | 12711 | TGTTCTTGACCGTGTT | 98 | 2039 |
| 959716 | N/A | N/A | 12719 | 12734 | AAGGTTAAACTACCGA | 6 | 2040 |
| 959726 | N/A | N/A | 12785 | 12800 | TACATGGTCTGCAAAT | 90 | 2041 |
| 959736 | N/A | N/A | 12838 | 12853 | CATTGCATTGCATAGC | 96 | 2042 |
| 959746 | N/A | N/A | 12888 | 12903 | AAAACCAATCCTGTTA | 47 | 2043 |
| 959756 | N/A | N/A | 12911 | 12926 | CATCTGCTTATAAAGC | 81 | 2044 |
| 959766 | N/A | N/A | 12967 | 12982 | AAACTTTGCAGCCTAT | 93 | 2045 |
| 959776 | N/A | N/A | 13376 | 13391 | AGACTTTGCAGGCACC | 90 | 2046 |
| 959786 | N/A | N/A | 13400 | 13415 | GGCTTGAATGTCACCC | 69 | 2047 |
| 959796 | N/A | N/A | 13697 | 13712 | AATGCTTGTCAAAAGG | 70 | 2048 |
| 959806 | N/A | N/A | 13717 | 13732 | CTTTACCAAGACCGCT | 82 | 2049 |
| 959816 | N/A | N/A | 13747 | 13762 | TAAGTGTTAGAACTAA | 30 | 2050 |
| 959826 | N/A | N/A | 14232 | 14247 | ACCAACTTTTAGTATT | 79 | 2051 |
| 959836 | N/A | N/A | 14419 | 14434 | CATCGACACAGCATCA | 59 | 2052 |
| 959846 | N/A | N/A | 14557 | 14572 | CCAAAGTCTTAATGTG | 53 | 2053 |
| 959856 | N/A | N/A | 14676 | 14691 | CCATCTCTTAGTGTCC | 88 | 2054 |
| 959866 | N/A | N/A | 15210 | 15225 | CTTAGGAATATTGCCA | 87 | 2055 |
| 959876 | N/A | N/A | 15770 | 15785 | AGGGTTAGTGTTGGTT | 89 | 2056 |
| 959886 | N/A | N/A | 17202 | 17217 | TCTGTTTGACTTAGTC | 70 | 2057 |
| 959896 | N/A | N/A | 17290 | 17305 | CTTAGGGCACCTCAAG | 30 | 2058 |
| 959906 | N/A | N/A | 17735 | 17750 | TAATCTGGTCATATGG | 43 | 2059 |
| 959916 | N/A | N/A | 18445 | 18460 | TGCTTACGGAGCATAG | 0 | 2060 |
| 959926 | N/A | N/A | 20472 | 20487 | CTCTAGACGGGAAGCT | 31 | 2061 |
| 959936 | N/A | N/A | 20601 | 20616 | GATCATCATTATTACC | 71 | 2062 |
| 959946 | N/A | N/A | 20652 | 20667 | TTGCAGTGCCCTGGCC | 31 | 2063 |
| 959956 | N/A | N/A | 20804 | 20819 | ATTCAGACCAGGGTAA | 87 | 2064 |
| 959966 | N/A | N/A | 20847 | 20862 | ATGCCTGTATTAGCTC | 65 | 2065 |
| 959976 | N/A | N/A | 20942 | 20957 | AGCCTCTTTGTAGCAG | 5 | 2066 |
| 959986 | N/A | N/A | 21008 | 21023 | GAGGTTATTTTAACAG | 69 | 2067 |
| 959996 | N/A | N/A | 21435 | 21450 | ATACTGAGAGGAAATG | 68 | 2068 |
| 960006 | N/A | N/A | 21539 | 21554 | ATGACATTTCAGAGTA | 88 | 2069 |
| 960016 | N/A | N/A | 22715 | 22730 | GTGTAAAGATGTGAGT | 84 | 2070 |
| 960026 | N/A | N/A | 24033 | 24048 | TGCTGCACTCAAAGAG | 0 | 2071 |
| 960038 | N/A | N/A | 19377 | 19392 | TCACAAGAGACTGGAC | 31 | 2072 |

TABLE 34
Inhibition of PNPLA3 mRNA by 3-10-3 cEt gapmers targeting SEQ ID NO: 1 and 2

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | PNPLA3 | SEQ |
| Compound | Start | Stop | Start | Stop | Sequence | % | ID |
| Number | Site | Site | Site | Site | (5′ to 3′) | Inhibition | NO |
| 915609 | 705 | 720 | 11923 | 11938 | TGATGGTTGTTTTGGC | 98 | 702 |
| 959277 | 481 | 496 | 6055 | 6070 | TGGTGGACATTGGCCG | 5 | 2073 |
| 959287 | 707 | 722 | 11925 | 11940 | GGTGATGGTTGTTTTG | 85 | 2074 |
| 959297 | 896 | 911 | 13632 | 13647 | GAATGCATCCAAATAT | 0 | 2075 |
| 959307 | 1070 | 1085 | 16190 | 16205 | GTGGTCTAGCAGCTCA | 66 | 2076 |
| 959317 | 1223 | 1238 | 19063 | 19078 | CATTACATAAGACATT | 46 | 2077 |
| 959327 | 1615 | 1630 | 25378 | 25393 | CCTCAAGTGACTCACA | 83 | 2078 |
| 959337 | 1651 | 1666 | 25414 | 25429 | CTTTAGCACCTCTGAA | 71 | 2079 |
| 959347 | 1835 | 1850 | 25598 | 25613 | ATTTCCCAACCAGCTG | 49 | 2080 |
| 959357 | 2099 | 2114 | 25862 | 25877 | TTATCATCTTTGCAGA | 48 | 2081 |
| 959367 | 2185 | 2200 | 25948 | 25963 | TTTTAGTTAGGTGAAA | 43 | 2082 |
| 959377 | 2263 | 2278 | 26026 | 26041 | CTCACTGATTCACATA | 79 | 2083 |
| 959387 | N/A | N/A | 4307 | 4322 | TGAGAAACAAACCCTC | 0 | 2084 |
| 959397 | N/A | N/A | 4739 | 4754 | TGTGCAAATGATCATG | 82 | 2085 |
| 959407 | N/A | N/A | 4859 | 4874 | TAGGCTCCTGGGACCT | 0 | 2086 |
| 959417 | N/A | N/A | 5277 | 5292 | ATCTTGGGATGCACAG | 89 | 2087 |
| 959427 | N/A | N/A | 5567 | 5582 | TCATGGCTTCCAGTGT | 78 | 2088 |
| 959437 | N/A | N/A | 5600 | 5615 | TTCAATGTGGCTTCTA | 96 | 2089 |
| 959447 | N/A | N/A | 5627 | 5642 | TTAATTCTACCTGTGT | 72 | 2090 |
| 959457 | N/A | N/A | 5706 | 5721 | TGAAATATCTCATTAG | 77 | 2091 |
| 959467 | N/A | N/A | 5805 | 5820 | TGTCTACTATGGGAGC | 83 | 2092 |
| 959477 | N/A | N/A | 5875 | 5890 | ATGGTAATACTTTTGG | 75 | 2093 |
| 959657 | N/A | N/A | 12148 | 12163 | ACTGGTAGCTCCTGGC | 78 | 2094 |
| 959667 | N/A | N/A | 12165 | 12180 | TGATTGTGCAGACAGT | 97 | 2095 |
| 959677 | N/A | N/A | 12203 | 12218 | TATTTCCGTTAACCAT | 91 | 2096 |
| 959687 | N/A | N/A | 12289 | 12304 | CTGAGTTTTAGGTCTG | 96 | 2097 |
| 959697 | N/A | N/A | 12671 | 12686 | GAATCATCTTAGTGGC | 94 | 2098 |
| 959707 | N/A | N/A | 12697 | 12712 | TTGTTCTTGACCGTGT | 98 | 2099 |
| 959717 | N/A | N/A | 12753 | 12768 | GTGGTAAGGCATACTA | 35 | 2100 |
| 959727 | N/A | N/A | 12789 | 12804 | GGTGTACATGGTCTGC | 97 | 2101 |
| 959737 | N/A | N/A | 12839 | 12854 | GCATTGCATTGCATAG | 92 | 2102 |
| 959747 | N/A | N/A | 12890 | 12905 | GGAAAACCAATCCTGT | 69 | 2103 |
| 959757 | N/A | N/A | 12927 | 12942 | AGCTGTCTCCTCTACT | 70 | 2104 |
| 959767 | N/A | N/A | 12968 | 12983 | CAAACTTTGCAGCCTA | 95 | 2105 |
| 959777 | N/A | N/A | 13377 | 13392 | GAGACTTTGCAGGCAC | 88 | 2106 |
| 959787 | N/A | N/A | 13402 | 13417 | TCGGCTTGAATGTCAC | 67 | 2107 |
| 959797 | N/A | N/A | 13700 | 13715 | GTAAATGCTTGTCAAA | 91 | 2108 |
| 959807 | N/A | N/A | 13720 | 13735 | AGTCTTTACCAAGACC | 0 | 2109 |
| 959817 | N/A | N/A | 13911 | 13926 | CATGACATCCCAGTTC | 29 | 2110 |
| 959827 | N/A | N/A | 14233 | 14248 | AACCAACTTTTAGTAT | 27 | 2111 |
| 959837 | N/A | N/A | 14420 | 14435 | CCATCGACACAGCATC | 89 | 2112 |
| 959847 | N/A | N/A | 14567 | 14582 | CTACTTATCCCCAAAG | 16 | 2113 |
| 959857 | N/A | N/A | 14677 | 14692 | GCCATCTCTTAGTGTC | 36 | 2114 |
| 959867 | N/A | N/A | 15211 | 15226 | CCTTAGGAATATTGCC | 75 | 2115 |
| 959877 | N/A | N/A | 15771 | 15786 | GAGGGTTAGTGTTGGT | 93 | 2116 |
| 959887 | N/A | N/A | 17218 | 17233 | CTGGTTTGTGGGTTCT | 75 | 2117 |
| 959897 | N/A | N/A | 17291 | 17306 | TCTTAGGGCACCTCAA | 53 | 2118 |
| 959907 | N/A | N/A | 17736 | 17751 | TTAATCTGGTCATATG | 0 | 2119 |
| 959917 | N/A | N/A | 18852 | 18867 | ACAAAAGCGACAAGGT | 33 | 2120 |
| 959927 | N/A | N/A | 20508 | 20523 | TTGAGTCTCCTGACCA | 65 | 2121 |
| 959937 | N/A | N/A | 20602 | 20617 | CGATCATCATTATTAC | 87 | 2122 |
| 959947 | N/A | N/A | 20653 | 20668 | ATTGCAGTGCCCTGGC | 69 | 2123 |
| 959957 | N/A | N/A | 20805 | 20820 | TATTCAGACCAGGGTA | 89 | 2124 |
| 959967 | N/A | N/A | 20848 | 20863 | GATGCCTGTATTAGCT | 72 | 2125 |
| 959977 | N/A | N/A | 20944 | 20959 | GCAGCCTCTTTGTAGC | 0 | 2126 |
| 959987 | N/A | N/A | 21010 | 21025 | CTGAGGTTATTTTAAC | 40 | 2127 |
| 959997 | N/A | N/A | 21436 | 21451 | TATACTGAGAGGAAAT | 47 | 2128 |
| 960007 | N/A | N/A | 21541 | 21556 | ATATGACATTTCAGAG | 91 | 2129 |
| 960017 | N/A | N/A | 22716 | 22731 | CGTGTAAAGATGTGAG | 87 | 2130 |
| 960027 | N/A | N/A | 24035 | 24050 | AATGCTGCACTCAAAG | 31 | 2131 |
| 960039 | N/A | N/A | 20215 | 20230 | TAACAAACTATGCCTA | 44 | 2132 |

TABLE 35
Inhibition of PNPLA3 mRNA by 3-10-3 cEt gapmers targeting SEQ ID NO: 1 and 2

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | PNPLA3 | SEQ |
| Compound | Start | Stop | Start | Stop | Sequence | % | ID |
| Number | Site | Site | Site | Site | (5′ to 3′) | Inhibition | NO |
| 915609 | 705 | 720 | 11923 | 11938 | TGATGGTTGTTTTGGC | 98 | 702 |
| 959274 | 439 | 454 | 6013 | 6028 | AGGAACTTGCTTAAGT | 73 | 2133 |
| 959284 | 695 | 710 | 11913 | 11928 | TTTGGCATCAATGAAG | 60 | 1928 |
| 959294 | 817 | 832 | 12035 | 12050 | TAGAGGTTCCCTGTGC | 77 | 1929 |
| 959304 | 1063 | 1078 | 16183 | 16198 | AGCAGCTCATCTCCCT | 62 | 2134 |
| 959314 | 1091 | 1106 | 16211 | 16226 | GGGCAGGATGCTGAGA | 13 | 1930 |
| 959324 | 1606 | 1621 | 25369 | 25384 | ACTCACAGACTCTTCT | 72 | 1931 |
| 959334 | 1647 | 1662 | 25410 | 25425 | AGCACCTCTGAAAGAA | 65 | 1932 |
| 959344 | 1678 | 1693 | 25441 | 25456 | CGGAGGTAGCTGCACA | 86 | 1933 |
| 959354 | 1926 | 1941 | 25689 | 25704 | ATTCTAAGAACCTCAT | 54 | 1934 |
| 959364 | 2181 | 2196 | 25944 | 25959 | AGTTAGGTGAAAAAGG | 92 | 2135 |
| 959374 | 2260 | 2275 | 26023 | 26038 | ACTGATTCACATAATA | 78 | 2136 |
| 959384 | N/A | N/A | 4303 | 4318 | AAACAAACCCTCCGTC | 2 | 1935 |
| 959394 | N/A | N/A | 4734 | 4749 | AAATGATCATGTGGCG | 94 | 1936 |
| 959404 | N/A | N/A | 4768 | 4783 | CTGACTTTTATTGTTG | 72 | 1937 |
| 959414 | N/A | N/A | 4871 | 4886 | GTGAGTGTACTTTAGG | 97 | 1938 |
| 959424 | N/A | N/A | 5393 | 5408 | AATGCTATCAGGTGCA | 31 | 1939 |
| 959434 | N/A | N/A | 5579 | 5594 | TGCACAATGACATCAT | 87 | 1940 |
| 959444 | N/A | N/A | 5621 | 5636 | CTACCTGTGTCTTTTA | 90 | 1941 |
| 959454 | N/A | N/A | 5647 | 5662 | ATATATTGGGCTCAAT | 80 | 1942 |
| 959464 | N/A | N/A | 5799 | 5814 | CTATGGGAGCCACATG | 24 | 2137 |
| 959474 | N/A | N/A | 5867 | 5882 | ACTTTTGGCAAGGCCA | 23 | 1943 |
| 959484 | N/A | N/A | 7211 | 7226 | CCGCAAACAAGGTTAA | 0 | 1944 |
| 959664 | N/A | N/A | 12157 | 12172 | CAGACAGTAACTGGTA | 96 | 2138 |
| 959674 | N/A | N/A | 12200 | 12215 | TTCCGTTAACCATCAA | 97 | 2139 |
| 959684 | N/A | N/A | 12283 | 12298 | TTTAGGTCTGGGTATA | 93 | 1945 |
| 959694 | N/A | N/A | 12325 | 12340 | CCCCCTGACTATATAA | 26 | 1946 |
| 959704 | N/A | N/A | 12693 | 12708 | TCTTGACCGTGTTTCC | 98 | 1947 |
| 959714 | N/A | N/A | 12716 | 12731 | GTTAAACTACCGAACG | 35 | 2140 |
| 959724 | N/A | N/A | 12763 | 12778 | CTATGGTAGAGTGGTA | 93 | 2141 |
| 959734 | N/A | N/A | 12834 | 12849 | GCATTGCATAGCCTTC | 97 | 1948 |
| 959744 | N/A | N/A | 12886 | 12901 | AACCAATCCTGTTAGA | 55 | 1949 |
| 959754 | N/A | N/A | 12909 | 12924 | TCTGCTTATAAAGCAC | 0 | 1950 |
| 959764 | N/A | N/A | 12937 | 12952 | GGACAATAAGAGCTGT | 91 | 2142 |
| 959774 | N/A | N/A | 13373 | 13388 | CTTTGCAGGCACCCCA | 72 | 1951 |
| 959784 | N/A | N/A | 13398 | 13413 | CTTGAATGTCACCCTT | 92 | 1952 |
| 959794 | N/A | N/A | 13532 | 13547 | AGCATCATTGGAAGAC | 92 | 2143 |
| 959804 | N/A | N/A | 13715 | 13730 | TTACCAAGACCGCTAG | 57 | 1953 |
| 959814 | N/A | N/A | 13744 | 13759 | GTGTTAGAACTAAGGC | 94 | 2144 |
| 959824 | N/A | N/A | 14228 | 14243 | ACTTTTAGTATTAAAG | 0 | 1954 |
| 959834 | N/A | N/A | 14306 | 14321 | TTTCTGAGCAGATAAA | 66 | 2145 |
| 959844 | N/A | N/A | 14555 | 14570 | AAAGTCTTAATGTGGA | 87 | 1955 |
| 959854 | N/A | N/A | 14670 | 14685 | CTTAGTGTCCCCATCC | 77 | 1956 |
| 959864 | N/A | N/A | 15208 | 15223 | TAGGAATATTGCCAGG | 89 | 2146 |
| 959874 | N/A | N/A | 15767 | 15782 | GTTAGTGTTGGTTTAT | 94 | 1957 |
| 959884 | N/A | N/A | 17199 | 17214 | GTTTGACTTAGTCCGT | 95 | 1958 |
| 959894 | N/A | N/A | 17228 | 17243 | AACTCTGTAGCTGGTT | 41 | 2147 |
| 959904 | N/A | N/A | 17603 | 17618 | TCTTGATAGTGAATGT | 73 | 2148 |
| 959914 | N/A | N/A | 18398 | 18413 | AATATGTTTGGAAGTC | 89 | 1959 |
| 959924 | N/A | N/A | 20292 | 20307 | CCAATCAGAGGAAGCC | 58 | 2149 |
| 959934 | N/A | N/A | 20518 | 20533 | AGTAACCAGATTGAGT | 81 | 1960 |
| 959944 | N/A | N/A | 20617 | 20632 | CTGTCTCTAATTTTAC | 75 | 2150 |
| 959954 | N/A | N/A | 20665 | 20680 | CACAGCTTAGAAATTG | 87 | 1961 |
| 959964 | N/A | N/A | 20844 | 20859 | CCTGTATTAGCTCAAT | 92 | 1962 |
| 959974 | N/A | N/A | 20940 | 20955 | CCTCTTTGTAGCAGAC | 83 | 1963 |
| 959984 | N/A | N/A | 21006 | 21021 | GGTTATTTTAACAGCT | 85 | 1964 |
| 959994 | N/A | N/A | 21412 | 21427 | ACCTGCAATTCTAGAC | 54 | 1965 |
| 960004 | N/A | N/A | 21537 | 21552 | GACATTTCAGAGTATA | 93 | 2151 |
| 960014 | N/A | N/A | 22710 | 22725 | AAGATGTGAGTGAAAT | 69 | 1966 |
| 960024 | N/A | N/A | 22770 | 22785 | AAAGGACATGACAGAC | 75 | 1967 |
| 960034 | N/A | N/A | 24613 | 24628 | GCAAATCGGATCTTTG | 32 | 2152 |

TABLE 36
Inhibition of PNPLA3 mRNA by 3-10-3 cEt gapmers targeting SEQ ID NO: 1 and 2

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | PNPLA3 | SEQ |
| Compound | Start | Stop | Start | Stop | Sequence | % | ID |
| Number | Site | Site | Site | Site | (5′ to 3′) | Inhibition | NO |
| 915609 | 705 | 720 | 11923 | 11938 | TGATGGTTGTTTTGGC | 98 | 702 |
| 959275 | 440 | 455 | 6014 | 6029 | GAGGAACTTGCTTAAG | 80 | 1968 |
| 959285 | 696 | 711 | 11914 | 11929 | TTTTGGCATCAATGAA | 63 | 1969 |
| 959295 | 823 | 838 | 12041 | 12056 | AGAAGGTAGAGGTTCC | 48 | 2153 |
| 959305 | 1066 | 1081 | 16186 | 16201 | TCTAGCAGCTCATCTC | 66 | 1970 |
| 959315 | 1093 | 1108 | 16213 | 16228 | CAGGGCAGGATGCTGA | 2 | 2154 |
| 959325 | 1608 | 1623 | 25371 | 25386 | TGACTCACAGACTCTT | 60 | 2155 |
| 959335 | 1649 | 1664 | 25412 | 25427 | TTAGCACCTCTGAAAG | 54 | 1971 |
| 959345 | 1804 | 1819 | 25567 | 25582 | ATGTATTAGAGTTAAG | 79 | 1972 |
| 959355 | 1927 | 1942 | 25690 | 25705 | CATTCTAAGAACCTCA | 68 | 1973 |
| 959365 | 2183 | 2198 | 25946 | 25961 | TTAGTTAGGTGAAAAA | 69 | 1974 |
| 959375 | 2261 | 2276 | 26024 | 26039 | CACTGATTCACATAAT | 73 | 1975 |
| 959385 | N/A | N/A | 4305 | 4320 | AGAAACAAACCCTCCG | 70 | 2156 |
| 959395 | N/A | N/A | 4737 | 4752 | TGCAAATGATCATGTG | 69 | 1976 |
| 959405 | N/A | N/A | 4769 | 4784 | GCTGACTTTTATTGTT | 83 | 1977 |
| 959415 | N/A | N/A | 4872 | 4887 | AGTGAGTGTACTTTAG | 94 | 1978 |
| 959425 | N/A | N/A | 5395 | 5410 | TTAATGCTATCAGGTG | 82 | 1979 |
| 959435 | N/A | N/A | 5580 | 5595 | ATGCACAATGACATCA | 84 | 1980 |
| 959445 | N/A | N/A | 5624 | 5639 | ATTCTACCTGTGTCTT | 95 | 1981 |
| 959455 | N/A | N/A | 5651 | 5666 | TTGGATATATTGGGCT | 97 | 1982 |
| 959465 | N/A | N/A | 5800 | 5815 | ACTATGGGAGCCACAT | 26 | 2157 |
| 959475 | N/A | N/A | 5868 | 5883 | TACTTTTGGCAAGGCC | 69 | 1983 |
| 959485 | N/A | N/A | 7697 | 7712 | GCACAGAGTAGGTTAA | 70 | 1984 |
| 959655 | N/A | N/A | 12146 | 12161 | TGGTAGCTCCTGGCAA | 50 | 1985 |
| 959665 | N/A | N/A | 12162 | 12177 | TTGTGCAGACAGTAAC | 87 | 2158 |
| 959675 | N/A | N/A | 12201 | 12216 | TTTCCGTTAACCATCA | 95 | 1986 |
| 959685 | N/A | N/A | 12284 | 12299 | TTTTAGGTCTGGGTAT | 81 | 2159 |
| 959695 | N/A | N/A | 12667 | 12682 | CATCTTAGTGGCTGGG | 91 | 1987 |
| 959705 | N/A | N/A | 12695 | 12710 | GTTCTTGACCGTGTTT | 97 | 1988 |
| 959715 | N/A | N/A | 12717 | 12732 | GGTTAAACTACCGAAC | 26 | 1989 |
| 959725 | N/A | N/A | 12783 | 12798 | CATGGTCTGCAAATTT | 89 | 1990 |
| 959735 | N/A | N/A | 12837 | 12852 | ATTGCATTGCATAGCC | 95 | 2160 |
| 959745 | N/A | N/A | 12887 | 12902 | AAACCAATCCTGTTAG | 54 | 1991 |
| 959755 | N/A | N/A | 12910 | 12925 | ATCTGCTTATAAAGCA | 43 | 1992 |
| 959765 | N/A | N/A | 12964 | 12979 | CTTTGCAGCCTATCCC | 95 | 2161 |
| 959775 | N/A | N/A | 13374 | 13389 | ACTTTGCAGGCACCCC | 86 | 1993 |
| 959785 | N/A | N/A | 13399 | 13414 | GCTTGAATGTCACCCT | 95 | 1994 |
| 959795 | N/A | N/A | 13534 | 13549 | TCAGCATCATTGGAAG | 60 | 2162 |
| 959805 | N/A | N/A | 13716 | 13731 | TTTACCAAGACCGCTA | 82 | 1995 |
| 959815 | N/A | N/A | 13745 | 13760 | AGTGTTAGAACTAAGG | 93 | 2163 |
| 959825 | N/A | N/A | 14230 | 14245 | CAACTTTTAGTATTAA | 9 | 1996 |
| 959835 | N/A | N/A | 14417 | 14432 | TCGACACAGCATCACC | 59 | 1997 |
| 959845 | N/A | N/A | 14556 | 14571 | CAAAGTCTTAATGTGG | 84 | 2164 |
| 959855 | N/A | N/A | 14671 | 14686 | TCTTAGTGTCCCCATC | 78 | 1998 |
| 959865 | N/A | N/A | 15209 | 15224 | TTAGGAATATTGCCAG | 91 | 1999 |
| 959875 | N/A | N/A | 15769 | 15784 | GGGTTAGTGTTGGTTT | 93 | 2000 |
| 959885 | N/A | N/A | 17200 | 17215 | TGTTTGACTTAGTCCG | 96 | 2165 |
| 959895 | N/A | N/A | 17288 | 17303 | TAGGGCACCTCAAGAA | 0 | 2001 |
| 959905 | N/A | N/A | 17734 | 17749 | AATCTGGTCATATGGT | 42 | 2166 |
| 959915 | N/A | N/A | 18400 | 18415 | CAAATATGTTTGGAAG | 55 | 2002 |
| 959925 | N/A | N/A | 20293 | 20308 | CCCAATCAGAGGAAGC | 57 | 2003 |
| 959935 | N/A | N/A | 20599 | 20614 | TCATCATTATTACCTG | 93 | 2004 |
| 959945 | N/A | N/A | 20651 | 20666 | TGCAGTGCCCTGGCCT | 40 | 2167 |
| 959955 | N/A | N/A | 20803 | 20818 | TTCAGACCAGGGTAAT | 93 | 2005 |
| 959965 | N/A | N/A | 20845 | 20860 | GCCTGTATTAGCTCAA | 88 | 2006 |
| 959975 | N/A | N/A | 20941 | 20956 | GCCTCTTTGTAGCAGA | 0 | 2007 |
| 959985 | N/A | N/A | 21007 | 21022 | AGGTTATTTTAACAGC | 94 | 2168 |
| 959995 | N/A | N/A | 21434 | 21449 | TACTGAGAGGAAATGA | 65 | 2008 |
| 960005 | N/A | N/A | 21538 | 21553 | TGACATTTCAGAGTAT | 87 | 2169 |
| 960015 | N/A | N/A | 22714 | 22729 | TGTAAAGATGTGAGTG | 75 | 2009 |
| 960025 | N/A | N/A | 22772 | 22787 | TCAAAGGACATGACAG | 78 | 2010 |
| 960037 | N/A | N/A | 22590 | 22605 | GAGCAACGAGGAAGGA | 64 | 2011 |

### Example 2: Dose-Dependent Antisense Inhibition of Human PNPLA3 in A431 Cells

Gapmers from Example 1 exhibiting significant in vitro inhibition of PNPLA3 mRNA were selected and tested at various doses in A431 cells. The antisense oligonucleotides were tested in a series of experiments that had similar culture conditions. The results for each experiment are presented in separate tables shown below. Cells were plated at a density of 10,000 cells per well and transfected free uptake with different concentrations of antisense oligonucleotide, as specified in the Tables below. After a treatment period of approximately 16 hours, RNA was isolated from the cells and PNPLA3 mRNA levels were measured by quantitative real-time PCR. Human primer probe set RTS36070 was used to measure mRNA levels. PNPLA3 mRNA levels were adjusted according to total RNA content, as measured by RIBOGREEN®. Results are presented as percent inhibition of PNPLA3, relative to untreated control cells.

The half maximal inhibitory concentration (IC₅₀) of each oligonucleotide is also presented. PNPLA3 mRNA levels were significantly reduced in a dose-dependent manner in antisense oligonucleotide treated cells.

TABLE 37
Multi-dose assay of 3-10-3 cEt gapmers in A431 cells

| | PNPLA3% Inhibition | | | | |
|---|---|---|---|---|---|
| Compound | 62.5 | 250 | 1,000 | 4,000 | IC₅₀ |
| Number | nM | nM | nM | nM | (μM) |
| 912712 | 27 | 67 | 76 | 74 | 0.2 |
| 912732 | 54 | 78 | 88 | 87 | <0.1 |
| 912733 | 45 | 74 | 85 | 88 | <0.1 |
| 912734 | 33 | 64 | 80 | 83 | 0.1 |
| 912756 | 46 | 72 | 89 | 92 | <0.1 |
| 912757 | 31 | 62 | 78 | 86 | 0.2 |
| 912758 | 38 | 70 | 85 | 90 | 0.1 |
| 912759 | 66 | 92 | 97 | 98 | <0.1 |
| 912772 | 46 | 63 | 79 | 88 | 0.1 |
| 912795 | 40 | 64 | 83 | 84 | 0.1 |
| 912812 | 43 | 81 | 88 | 88 | <0.1 |
| 912822 | 81 | 83 | 92 | 86 | <0.1 |
| 912823 | 67 | 80 | 91 | 86 | <0.1 |
| 912825 | 58 | 80 | 86 | 88 | <0.1 |
| 912834 | 37 | 75 | 81 | 84 | 0.1 |
| 912841 | 17 | 62 | 79 | 69 | 0.3 |
| 912847 | 70 | 83 | 90 | 91 | <0.1 |
| 912848 | 80 | 89 | 90 | 90 | <0.1 |
| 912855 | 48 | 62 | 77 | 80 | 0.1 |

TABLE 38
Multi-dose assay of 3-10-3 cEt gapmers in A431 cells

| | PNPLA3% Inhibition | | | | |
|---|---|---|---|---|---|
| Compound | 62.5 | 250 | 1,000 | 4,000 | IC₅₀ |
| Number | nM | nM | nM | nM | (μM) |
| 912759 | 68 | 94 | 94 | 98 | <0.1 |
| 912813 | 57 | 84 | 90 | 87 | <0.1 |
| 912856 | 60 | 81 | 91 | 88 | <0.1 |
| 912859 | 48 | 79 | 81 | 72 | <0.1 |
| 912864 | 60 | 88 | 90 | 90 | <0.1 |
| 912870 | 67 | 81 | 91 | 94 | <0.1 |
| 912871 | 21 | 67 | 84 | 89 | 0.2 |
| 912872 | 18 | 73 | 90 | 92 | 0.2 |
| 912876 | 43 | 70 | 87 | 92 | 0.1 |
| 912933 | 68 | 89 | 90 | 90 | <0.1 |
| 912940 | 86 | 91 | 95 | 96 | <0.1 |
| 912941 | 87 | 94 | 96 | 96 | <0.1 |
| 912952 | 68 | 85 | 90 | 91 | <0.1 |
| 912953 | 80 | 90 | 95 | 93 | <0.1 |
| 912964 | 59 | 78 | 88 | 91 | <0.1 |
| 912973 | 53 | 70 | 87 | 91 | <0.1 |
| 912980 | 54 | 77 | 84 | 88 | <0.1 |
| 912985 | 23 | 61 | 81 | 87 | 0.2 |
| 912988 | 65 | 83 | 86 | 89 | <0.1 |

TABLE 39
Multi-dose assay of 3-10-3 cEt gapmers in A431 cells

| | PNPLA3% Inhibition | | | | |
|---|---|---|---|---|---|
| Compound | 62.5 | 250 | 1,000 | 4,000 | IC₅₀ |
| Number | nM | nM | nM | nM | (μM) |
| 912759 | 72 | 95 | 97 | 99 | <0.1 |
| 912874 | 78 | 90 | 96 | 97 | <0.1 |
| 912875 | 64 | 83 | 92 | 94 | <0.1 |
| 912886 | 49 | 78 | 85 | 92 | <0.1 |
| 912931 | 68 | 88 | 94 | 95 | <0.1 |
| 912934 | 57 | 83 | 90 | 92 | <0.1 |
| 912936 | 50 | 78 | 89 | 89 | <0.1 |
| 912938 | 57 | 73 | 85 | 87 | <0.1 |
| 912943 | 64 | 84 | 90 | 93 | <0.1 |
| 912954 | 80 | 92 | 93 | 94 | <0.1 |
| 912970 | 44 | 73 | 86 | 90 | <0.1 |
| 912986 | 56 | 78 | 91 | 92 | <0.1 |
| 912987 | 79 | 90 | 92 | 88 | <0.1 |
| 912992 | 21 | 59 | 74 | 81 | 0.3 |
| 915603 | 50 | 88 | 96 | 98 | <0.1 |
| 915623 | 81 | 96 | 98 | 98 | <0.1 |
| 915643 | 67 | 89 | 94 | 96 | <0.1 |
| 916602 | 79 | 92 | 95 | 96 | <0.1 |
| 916642 | 44 | 83 | 91 | 93 | <0.1 |

TABLE 40
Multi-dose assay of 3-10-3 cEt gapmers in A431 cells

| | PNPLA3% Inhibition | | | | |
|---|---|---|---|---|---|
| Compound | 62.5 | 250 | 1,000 | 4,000 | IC₅₀ |
| Number | nM | nM | nM | nM | (μM) |
| 912759 | 73 | 94 | 98 | 99 | <0.1 |
| 915484 | 67 | 87 | 93 | 95 | <0.1 |
| 915543 | 34 | 69 | 87 | 90 | 0.1 |
| 915604 | 54 | 78 | 91 | 95 | <0.1 |
| 915763 | 63 | 80 | 87 | 87 | <0.1 |
| 915904 | 50 | 83 | 92 | 94 | <0.1 |
| 915923 | 63 | 74 | 82 | 87 | <0.1 |
| 916183 | 33 | 78 | 89 | 91 | 0.1 |
| 916303 | 58 | 73 | 84 | 91 | <0.1 |
| 916343 | 15 | 72 | 76 | 87 | 0.2 |
| 916563 | 46 | 74 | 90 | 95 | <0.1 |
| 916582 | 48 | 74 | 89 | 91 | <0.1 |
| 916623 | 64 | 81 | 91 | 94 | <0.1 |
| 916702 | 45 | 70 | 78 | 79 | <0.1 |
| 916761 | 46 | 75 | 85 | 88 | <0.1 |
| 916781 | 55 | 79 | 86 | 87 | <0.1 |
| 916782 | 62 | 87 | 91 | 93 | <0.1 |
| 916802 | 66 | 88 | 94 | 91 | <0.1 |
| 916822 | 29 | 72 | 83 | 87 | 0.1 |

TABLE 41
Multi-dose assay of 3-10-3 cEt gapmers in A431 cells

| | PNPLA3% Inhibition | | | | |
|---|---|---|---|---|---|
| Compound | 62.5 | 250 | 1,000 | 4,000 | IC₅₀ |
| Number | nM | nM | nM | nM | (μM) |
| 912759 | 72 | 95 | 98 | 99 | <0.1 |
| 915525 | 51 | 76 | 88 | 84 | <0.1 |
| 915546 | 39 | 79 | 90 | 94 | 0.1 |
| 915605 | 59 | 84 | 96 | 96 | <0.1 |
| 915606 | 74 | 94 | 99 | 98 | <0.1 |
| 915625 | 72 | 82 | 91 | 95 | <0.1 |
| 915944 | 36 | 71 | 75 | 83 | 0.1 |
| 916065 | 36 | 62 | 78 | 79 | 0.1 |
| 916144 | 71 | 86 | 90 | 92 | <0.1 |
| 916163 | 36 | 67 | 81 | 74 | 0.1 |
| 916164 | 82 | 88 | 89 | 92 | <0.1 |
| 916184 | 60 | 79 | 87 | 89 | <0.1 |
| 916304 | 46 | 65 | 80 | 84 | 0.1 |
| 916324 | 57 | 77 | 87 | 92 | <0.1 |
| 916344 | 41 | 70 | 83 | 88 | 0.1 |
| 916564 | 38 | 66 | 88 | 94 | 0.1 |
| 916604 | 67 | 87 | 95 | 96 | <0.1 |
| 916624 | 43 | 59 | 79 | 87 | 0.1 |
| 916803 | 67 | 84 | 93 | 92 | <0.1 |

TABLE 42
Multi-dose assay of 3-10-3 cEt gapmers in A431 cells

| | PNPLA3% Inhibition | | | | |
|---|---|---|---|---|---|
| Compound | 62.5 | 250 | 1,000 | 4,000 | IC₅₀ |
| Number | nM | nM | nM | nM | (μM) |
| 912759 | 70 | 94 | 98 | 99 | <0.1 |
| 915486 | 35 | 64 | 82 | 90 | 0.1 |
| 915487 | 62 | 89 | 94 | 95 | <0.1 |
| 915626 | 67 | 83 | 92 | 94 | <0.1 |
| 915786 | 65 | 84 | 88 | 88 | <0.1 |
| 916145 | 53 | 66 | 85 | 87 | <0.1 |
| 916146 | 62 | 77 | 86 | 86 | <0.1 |
| 916165 | 71 | 86 | 89 | 88 | <0.1 |
| 916166 | 71 | 83 | 87 | 88 | <0.1 |
| 916305 | 57 | 86 | 90 | 92 | <0.1 |
| 916306 | 86 | 96 | 98 | 98 | <0.1 |
| 916325 | 59 | 78 | 83 | 86 | <0.1 |
| 916345 | 21 | 47 | 67 | 73 | 0.4 |
| 916545 | 63 | 88 | 95 | 94 | <0.1 |
| 916546 | 66 | 85 | 92 | 95 | <0.1 |
| 916625 | 47 | 71 | 84 | 92 | <0.1 |
| 916706 | 22 | 65 | 80 | 85 | 0.2 |
| 916765 | 67 | 85 | 92 | 93 | <0.1 |
| 916845 | 38 | 71 | 80 | 87 | 0.1 |

TABLE 43
Multi-dose assay of 3-10-3 cEt gapmers in A431 cells

| | PNPLA3% Inhibition | | | | |
|---|---|---|---|---|---|
| Compound | 62.5 | 250 | 1,000 | 4,000 | IC₅₀ |
| Number | nM | nM | nM | nM | (μM) |
| 912759 | 97 | 99 | 100 | 100 | <0.1 |
| 915608 | 66 | 91 | 97 | 97 | <0.1 |
| 915609 | 71 | 97 | 99 | 99 | <0.1 |
| 915627 | 0 | 26 | 53 | 62 | 1.3 |
| 915768 | 39 | 69 | 86 | 91 | 0.1 |
| 915908 | 49 | 70 | 80 | 85 | <0.1 |
| 915987 | 47 | 60 | 75 | 78 | 0.1 |
| 916008 | 45 | 69 | 84 | 83 | <0.1 |
| 916187 | 71 | 82 | 88 | 92 | <0.1 |
| 916247 | 34 | 72 | 83 | 84 | 0.1 |
| 916287 | 31 | 70 | 90 | 91 | 0.1 |
| 916547 | 79 | 93 | 97 | 97 | <0.1 |
| 916566 | 8 | 45 | 73 | 81 | 0.5 |
| 916586 | 47 | 67 | 89 | 91 | <0.1 |
| 916587 | 48 | 81 | 90 | 94 | <0.1 |
| 916606 | 18 | 64 | 87 | 90 | 0.2 |
| 916607 | 72 | 94 | 94 | 95 | <0.1 |
| 916627 | 18 | 51 | 82 | 79 | 0.3 |
| 916805 | 18 | 65 | 78 | 81 | 0.2 |

TABLE 44
Multi-dose assay of 3-10-3 cEt gapmers in A431 cells

| | PNPLA3 % Inhibition | | | | |
|---|---|---|---|---|---|
| Compound | 62.5 | 250 | 1,000 | 4,000 | IC₅₀ |
| Number | nM | nM | nM | nM | (μM) |
| 912759 | 64 | 92 | 97 | 99 | <0.1 |
| 915610 | 74 | 94 | 98 | 99 | <0.1 |
| 915789 | 35 | 72 | 82 | 85 | 0.1 |
| 915909 | 52 | 69 | 82 | 86 | <0.1 |
| 915929 | 13 | 32 | 60 | 59 | 1.0 |
| 915969 | 39 | 54 | 74 | 74 | 0.2 |
| 915989 | 46 | 67 | 81 | 86 | 0.1 |
| 916069 | 24 | 59 | 75 | 56 | 0.3 |
| 916148 | 42 | 71 | 85 | 80 | <0.1 |
| 916168 | 28 | 54 | 74 | 68 | 0.3 |
| 916188 | 22 | 42 | 72 | 70 | 0.4 |
| 916309 | 30 | 77 | 91 | 96 | 0.1 |
| 916348 | 41 | 57 | 65 | 73 | 0.1 |
| 916549 | 64 | 85 | 94 | 96 | <0.1 |
| 916568 | 54 | 66 | 81 | 87 | <0.1 |
| 916569 | 60 | 86 | 92 | 95 | <0.1 |
| 916728 | 22 | 50 | 68 | 73 | 0.4 |
| 916788 | 60 | 89 | 94 | 96 | <0.1 |
| 916848 | 49 | 75 | 89 | 95 | <0.1 |

TABLE 45
Multi-dose assay of 3-10-3 cEt gapmers in A431 cells

| | PNPLA3 % Inhibition | | | | |
|---|---|---|---|---|---|
| Compound | 62.5 | 250 | 1,000 | 4,000 | IC₅₀ |
| Number | nM | nM | nM | nM | (μM) |
| 912759 | 81 | 93 | 98 | 100 | <0.1 |
| 915390 | 9 | 40 | 67 | 77 | 0.6 |
| 915611 | 46 | 80 | 93 | 88 | <0.1 |
| 915630 | 52 | 69 | 81 | 82 | <0.1 |
| 915910 | 44 | 64 | 79 | 80 | 0.1 |
| 915931 | 83 | 88 | 89 | 86 | <0.1 |
| 916149 | 73 | 87 | 89 | 83 | <0.1 |
| 916150 | 51 | 68 | 77 | 84 | <0.1 |
| 916189 | 60 | 73 | 77 | 79 | <0.1 |
| 916310 | 45 | 77 | 88 | 95 | <0.1 |
| 916330 | 48 | 67 | 84 | 86 | <0.1 |
| 916550 | 62 | 85 | 94 | 97 | <0.1 |
| 916570 | 89 | 96 | 98 | 98 | <0.1 |
| 916629 | 26 | 53 | 73 | 86 | 0.3 |
| 916630 | 52 | 68 | 87 | 91 | <0.1 |
| 916670 | 43 | 77 | 78 | 85 | <0.1 |
| 916730 | 61 | 74 | 82 | 86 | <0.1 |
| 916768 | 35 | 57 | 67 | 72 | 0.2 |
| 916789 | 79 | 92 | 96 | 96 | <0.1 |

TABLE 46
Multi-dose assay of 3-10-3 cEt gapmers in A431 cells

| | PNPLA3 % Inhibition | | | | |
|---|---|---|---|---|---|
| Compound | 62.5 | 250 | 1,000 | 4,000 | IC₅₀ |
| Number | nM | nM | nM | nM | (μM) |
| 912759 | 76 | 94 | 96 | 99 | <0.1 |
| 915532 | 31 | 66 | 82 | 92 | 0.1 |
| 915612 | 54 | 77 | 86 | 90 | <0.1 |
| 915732 | 42 | 63 | 80 | 84 | 0.1 |
| 915932 | 45 | 71 | 88 | 89 | <0.1 |
| 915951 | 26 | 58 | 71 | 74 | 0.3 |
| 915991 | 67 | 84 | 85 | 85 | <0.1 |
| 915992 | 54 | 78 | 86 | 87 | <0.1 |
| 916112 | 35 | 67 | 76 | 78 | 0.1 |
| 916151 | 51 | 79 | 87 | 90 | <0.1 |
| 916311 | 36 | 70 | 81 | 87 | 0.1 |
| 916331 | 56 | 85 | 93 | 95 | <0.1 |
| 916332 | 82 | 91 | 94 | 96 | <0.1 |
| 916390 | 30 | 41 | 68 | 64 | 0.5 |
| 916552 | 79 | 93 | 96 | 97 | <0.1 |
| 916571 | 53 | 78 | 90 | 94 | <0.1 |
| 916631 | 48 | 77 | 86 | 90 | <0.1 |
| 916651 | 81 | 89 | 94 | 95 | <0.1 |
| 916711 | 37 | 66 | 85 | 91 | 0.1 |

TABLE 47
Multi-dose assay of 3-10-3 cEt gapmers in A431 cells

| | PNPLA3 % Inhibition | | | | |
|---|---|---|---|---|---|
| Compound | 62.5 | 250 | 1,000 | 4,000 | IC₅₀ |
| Number | nM | nM | nM | nM | (μM) |
| 912759 | 58 | 90 | 98 | 99 | <0.1 |
| 915474 | 51 | 79 | 90 | 93 | <0.1 |
| 915493 | 48 | 58 | 83 | 80 | 0.1 |
| 915494 | 46 | 73 | 86 | 90 | <0.1 |
| 915674 | 49 | 72 | 89 | 93 | <0.1 |
| 915933 | 40 | 63 | 75 | 79 | 0.1 |
| 916153 | 68 | 86 | 89 | 91 | <0.1 |
| 916172 | 85 | 89 | 87 | 87 | <0.1 |
| 916173 | 81 | 90 | 91 | 88 | <0.1 |
| 916292 | 64 | 83 | 92 | 92 | <0.1 |
| 916312 | 60 | 84 | 91 | 92 | <0.1 |
| 916333 | 75 | 92 | 96 | 96 | <0.1 |
| 916572 | 29 | 62 | 79 | 88 | 0.2 |
| 916592 | 52 | 74 | 89 | 90 | <0.1 |
| 916593 | 25 | 67 | 83 | 93 | 0.2 |
| 916613 | 46 | 75 | 89 | 92 | <0.1 |
| 916652 | 65 | 83 | 91 | 88 | <0.1 |
| 916672 | 73 | 89 | 93 | 90 | <0.1 |
| 916772 | 50 | 61 | 83 | 89 | <0.1 |

TABLE 48
Multi-dose assay of 3-10-3 cEt gapmers in A431 cells

| | PNPLA3 % Inhibition | | | | |
|---|---|---|---|---|---|
| Compound | 62.5 | 250 | 1,000 | 4,000 | IC₅₀ |
| Number | nM | nM | nM | nM | (μM) |
| 912759 | 50 | 89 | 96 | 99 | <0.1 |
| 915534 | 0 | 33 | 66 | 57 | 1.1 |
| 915535 | 51 | 81 | 92 | 96 | <0.1 |
| 915634 | 18 | 67 | 79 | 84 | 0.2 |
| 915635 | 44 | 72 | 86 | 91 | 0.1 |
| 915675 | 36 | 68 | 82 | 90 | 0.1 |
| 915735 | 45 | 68 | 73 | 84 | 0.1 |
| 915936 | 36 | 67 | 78 | 83 | 0.1 |
| 915995 | 78 | 87 | 90 | 89 | <0.1 |
| 915996 | 83 | 91 | 93 | 92 | <0.1 |
| 916174 | 80 | 84 | 86 | 81 | <0.1 |
| 916175 | 55 | 82 | 86 | 89 | <0.1 |
| 916334 | 50 | 82 | 92 | 94 | <0.1 |
| 916335 | 52 | 76 | 89 | 93 | <0.1 |
| 916575 | 62 | 88 | 93 | 93 | <0.1 |
| 916753 | 49 | 69 | 76 | 74 | <0.1 |
| 916774 | 49 | 72 | 86 | 91 | <0.1 |
| 916794 | 26 | 64 | 85 | 85 | 0.2 |
| 916873 | 16 | 49 | 72 | 82 | 0.4 |

TABLE 49
Multi-dose assay of 3-10-3 cEt gapmers in A431 cells

| | PNPLA3 % Inhibition | | | | |
|---|---|---|---|---|---|
| Compound | 62.5 | 250 | 1,000 | 4,000 | IC₅₀ |
| Number | nM | nM | nM | nM | (μM) |
| 912759 | 57 | 90 | 97 | 99 | <0.1 |
| 915477 | 47 | 65 | 88 | 93 | 0.1 |
| 915478 | 47 | 78 | 90 | 95 | <0.1 |
| 915497 | 63 | 68 | 79 | 86 | <0.1 |
| 915637 | 67 | 91 | 97 | 98 | <0.1 |
| 916037 | 15 | 47 | 70 | 61 | 0.6 |
| 916236 | 80 | 87 | 90 | 88 | <0.1 |
| 916336 | 52 | 67 | 81 | 87 | <0.1 |
| 916576 | 50 | 76 | 89 | 93 | <0.1 |
| 916596 | 55 | 82 | 93 | 94 | <0.1 |
| 916636 | 42 | 71 | 87 | 90 | 0.1 |
| 916637 | 56 | 85 | 90 | 93 | <0.1 |
| 916715 | 27 | 38 | 68 | 68 | 0.5 |
| 916716 | 35 | 77 | 89 | 93 | 0.1 |
| 916796 | 14 | 62 | 84 | 89 | 0.3 |
| 916814 | 22 | 44 | 70 | 79 | 0.4 |
| 916815 | 56 | 79 | 87 | 89 | <0.1 |
| 916816 | 33 | 72 | 83 | 93 | 0.1 |
| 916874 | 5 | 34 | 61 | 70 | 0.8 |

TABLE 50
Multi-dose assay of 3-10-3 cEt gapmers in A431 cells

| | PNPLA3 % Inhibition | | | | |
|---|---|---|---|---|---|
| Compound | 62.5 | 250 | 1,000 | 4,000 | IC₅₀ |
| Number | nM | nM | nM | nM | (μM) |
| 912759 | 56 | 91 | 97 | 100 | <0.1 |
| 915479 | 38 | 70 | 89 | 94 | 0.1 |
| 915618 | 42 | 63 | 75 | 85 | 0.1 |
| 915619 | 65 | 87 | 96 | 97 | <0.1 |
| 915638 | 31 | 64 | 80 | 82 | 0.2 |
| 915639 | 33 | 78 | 88 | 93 | 0.1 |
| 915778 | 41 | 50 | 78 | 87 | 0.2 |
| 916058 | 26 | 34 | 73 | 81 | 0.4 |
| 916177 | 38 | 55 | 83 | 82 | 0.1 |
| 916238 | 84 | 91 | 93 | 93 | <0.1 |
| 916298 | 79 | 87 | 92 | 94 | <0.1 |
| 916318 | 59 | 71 | 91 | 94 | <0.1 |
| 916338 | 71 | 91 | 94 | 92 | <0.1 |
| 916558 | 73 | 89 | 94 | 94 | <0.1 |
| 916577 | 41 | 66 | 78 | 82 | 0.1 |
| 916578 | 69 | 85 | 91 | 93 | <0.1 |
| 916638 | 46 | 84 | 90 | 92 | <0.1 |
| 916757 | 33 | 60 | 82 | 88 | 0.2 |
| 916817 | 31 | 67 | 82 | 87 | 0.1 |

TABLE 51
Multi-dose assay of 3-10-3 cEt gapmers in A431 cells

| | PNPLA3 % Inhibition | | | | |
|---|---|---|---|---|---|
| Compound | 62.5 | 250 | 1,000 | 4,000 | IC₅₀ |
| Number | nM | nM | nM | nM | (μM) |
| 841947 | 50 | 78 | 89 | 92 | <0.1 |
| 912759 | 75 | 50 | 85 | 99 | <0.1 |
| 912986 | 54 | 78 | 90 | 95 | <0.1 |
| 915480 | 61 | 87 | 94 | 97 | <0.1 |
| 915519 | 47 | 77 | 86 | 85 | <0.1 |
| 915620 | 46 | 75 | 88 | 91 | <0.1 |
| 915780 | 24 | 76 | 92 | 94 | 0.1 |
| 915920 | 23 | 65 | 79 | 82 | 0.2 |
| 916020 | 45 | 80 | 85 | 81 | <0.1 |
| 916299 | 59 | 87 | 92 | 93 | <0.1 |
| 916339 | 88 | 95 | 97 | 97 | <0.1 |
| 916340 | 83 | 96 | 97 | 98 | <0.1 |
| 916559 | 41 | 68 | 83 | 89 | 0.1 |
| 916579 | 71 | 86 | 96 | 96 | <0.1 |
| 916580 | 59 | 90 | 95 | 96 | <0.1 |
| 916618 | 10 | 47 | 70 | 78 | 0.5 |
| 916639 | 38 | 58 | 80 | 86 | 0.1 |
| 916778 | 68 | 87 | 92 | 94 | <0.1 |
| 916818 | 60 | 77 | 90 | 91 | <0.1 |

TABLE 52
Multi-dose assay of 3-10-3 cEt gapmers in A431 cells

| | PNPLA3 % Inhibition | | | | |
|---|---|---|---|---|---|
| Compound | 62.5 | 250 | 1,000 | 4,000 | IC₅₀ |
| Number | nM | nM | nM | nM | (μM) |
| 912759 | 60 | 0 | 85 | 99 | 0.3 |
| 915541 | 48 | 71 | 89 | 91 | <0.1 |
| 915542 | 50 | 72 | 86 | 93 | <0.1 |
| 915601 | 8 | 53 | 84 | 84 | 0.3 |
| 915602 | 1 | 56 | 77 | 91 | 0.4 |
| 915621 | 21 | 54 | 75 | 80 | 0.3 |
| 915622 | 0 | 44 | 73 | 84 | 0.5 |
| 915922 | 27 | 64 | 79 | 85 | 0.2 |
| 916042 | 6 | 57 | 89 | 88 | 0.3 |
| 916140 | 43 | 82 | 90 | 89 | <0.1 |
| 916141 | 72 | 88 | 93 | 91 | <0.1 |
| 916180 | 33 | 62 | 69 | 83 | 0.2 |
| 916181 | 53 | 80 | 89 | 92 | <0.1 |
| 916341 | 0 | 78 | 94 | 94 | 0.3 |
| 916560 | 72 | 91 | 95 | 94 | <0.1 |
| 916581 | 38 | 76 | 91 | 91 | 0.1 |
| 916601 | 44 | 80 | 88 | 90 | <0.1 |
| 916701 | 61 | 83 | 91 | 93 | <0.1 |
| 916780 | 75 | 91 | 93 | 94 | <0.1 |

TABLE 53
Multi-dose assay of 3-10-3 cEt gapmers in A431 cells

| | PNPLA3 % Inhibition | | | | |
|---|---|---|---|---|---|
| Compound | 15.6 | 62.5 | 250 | 1,000 | IC₅₀ |
| Number | nM | nM | nM | nM | (μM) |
| 915609 | 52 | 86 | 96 | 99 | <0.01 |
| 959430 | 42 | 76 | 90 | 95 | <0.01 |
| 959440 | 39 | 73 | 92 | 98 | 0.02 |
| 959470 | 46 | 73 | 89 | 94 | <0.01 |
| 959670 | 52 | 90 | 96 | 98 | <0.01 |
| 959680 | 50 | 75 | 91 | 96 | <0.01 |
| 959730 | 83 | 96 | 98 | 98 | <0.01 |
| 959740 | 50 | 70 | 90 | 96 | <0.01 |
| 959820 | 40 | 69 | 85 | 92 | 0.02 |
| 959830 | 46 | 69 | 93 | 97 | 0.02 |
| 959880 | 34 | 62 | 85 | 93 | 0.03 |
| 960010 | 48 | 78 | 92 | 95 | <0.01 |

TABLE 54
Multi-dose assay of 3-10-3 cEt gapmers in A431 cells

| | PNPLA3 % Inhibition | | | | |
|---|---|---|---|---|---|
| Compound | 15.6 | 62.5 | 250 | 1,000 | IC₅₀ |
| Number | nM | nM | nM | nM | (μM) |
| 915609 | 53 | 87 | 98 | 99 | <0.01 |
| 959271 | 55 | 74 | 91 | 91 | <0.01 |
| 959360 | 7 | 43 | 79 | 85 | 0.1 |
| 959361 | 60 | 87 | 93 | 94 | <0.01 |
| 959411 | 56 | 76 | 91 | 94 | <0.01 |
| 959441 | 50 | 81 | 93 | 97 | <0.01 |
| 959460 | 0 | 29 | 75 | 90 | 0.2 |
| 959701 | 62 | 91 | 97 | 98 | <0.01 |
| 959721 | 80 | 94 | 97 | 97 | <0.01 |
| 959731 | 25 | 64 | 82 | 91 | 0.05 |
| 959741 | 41 | 65 | 83 | 91 | 0.02 |
| 959750 | 0 | 26 | 65 | 87 | 0.2 |
| 959761 | 28 | 60 | 84 | 91 | 0.05 |
| 959781 | 39 | 58 | 75 | 87 | 0.04 |
| 959911 | 20 | 54 | 78 | 90 | 0.1 |
| 959921 | 37 | 61 | 83 | 91 | 0.03 |
| 959931 | 48 | 72 | 89 | 92 | <0.01 |
| 959960 | 11 | 51 | 79 | 90 | 0.1 |
| 959961 | 38 | 64 | 85 | 92 | 0.03 |

TABLE 55
Multi-dose assay of 3-10-3 cEt gapmers in A431 cells

| | PNPLA3 % Inhibition | | | | |
|---|---|---|---|---|---|
| Compound | 15.6 | 62.5 | 250 | 1,000 | IC₅₀ |
| Number | nM | nM | nM | nM | (μM) |
| 915609 | 11 | 71 | 93 | 98 | 0.1 |
| 959412 | 52 | 77 | 90 | 94 | <0.01 |
| 959413 | 34 | 82 | 95 | 97 | 0.02 |
| 959422 | 15 | 50 | 80 | 87 | 0.1 |
| 959432 | 33 | 60 | 86 | 95 | 0.04 |
| 959662 | 0 | 53 | 84 | 92 | 0.1 |
| 959672 | 54 | 85 | 95 | 97 | <0.01 |
| 959673 | 18 | 62 | 88 | 95 | 0.1 |
| 959682 | 46 | 77 | 90 | 91 | <0.01 |
| 959702 | 39 | 71 | 91 | 96 | 0.02 |
| 959703 | 81 | 96 | 99 | 99 | <0.01 |
| 959712 | 4 | 30 | 75 | 92 | 0.1 |
| 959713 | 0 | 53 | 86 | 96 | 0.1 |
| 959722 | 33 | 80 | 90 | 94 | 0.02 |
| 959733 | 31 | 68 | 92 | 96 | 0.03 |
| 959782 | 35 | 63 | 86 | 94 | 0.03 |
| 959872 | 29 | 64 | 77 | 89 | 0.04 |
| 959912 | 25 | 69 | 89 | 92 | 0.04 |
| 959982 | 21 | 61 | 83 | 91 | 0.1 |

TABLE 56
Multi-dose assay of 3-10-3 cEt gapmers in A431 cells

| | PNPLA3 % Inhibition | | | | |
|---|---|---|---|---|---|
| Compound | 15.6 | 62.5 | 250 | 1,000 | IC₅₀ |
| Number | nM | nM | nM | nM | (μM) |
| 915609 | 2 | 73 | 93 | 98 | 0.1 |
| 959363 | 49 | 82 | 91 | 91 | <0.01 |
| 959393 | 38 | 71 | 87 | 95 | 0.02 |
| 959394 | 27 | 73 | 91 | 97 | 0.03 |
| 959414 | 69 | 94 | 98 | 99 | <0.01 |
| 959664 | 51 | 77 | 95 | 98 | <0.01 |
| 959674 | 43 | 74 | 95 | 98 | 0.02 |
| 959683 | 14 | 71 | 90 | 96 | 0.05 |
| 959704 | 57 | 92 | 98 | 99 | <0.01 |
| 959724 | 0 | 68 | 90 | 95 | 0.1 |
| 959734 | 71 | 93 | 98 | 98 | <0.01 |
| 959814 | 24 | 76 | 90 | 95 | 0.03 |
| 959873 | 38 | 53 | 83 | 90 | 0.04 |
| 959874 | 51 | 82 | 95 | 97 | <0.01 |
| 959884 | 44 | 77 | 94 | 97 | <0.01 |
| 959913 | 18 | 50 | 85 | 92 | 0.1 |
| 959953 | 6 | 51 | 85 | 92 | 0.1 |
| 959983 | 22 | 54 | 81 | 92 | 0.1 |
| 960004 | 10 | 71 | 92 | 96 | 0.1 |

TABLE 57
Multi-dose assay of 3-10-3 cEt gapmers in A431 cells

| | PNPLA3 % Inhibition | | | | |
|---|---|---|---|---|---|
| Compound | 15.6 | 62.5 | 250 | 1,000 | IC₅₀ |
| Number | nM | nM | nM | nM | (μM) |
| 915609 | 12 | 69 | 93 | 98 | 0.1 |
| 959364 | 32 | 72 | 89 | 91 | 0.03 |
| 959415 | 21 | 70 | 91 | 96 | 0.04 |
| 959444 | 6 | 47 | 82 | 91 | 0.1 |
| 959445 | 32 | 70 | 92 | 97 | 0.03 |
| 959455 | 61 | 87 | 95 | 97 | <0.01 |
| 959675 | 20 | 56 | 80 | 94 | 0.1 |
| 959684 | 8 | 47 | 83 | 86 | 0.1 |
| 959705 | 77 | 95 | 98 | 99 | <0.01 |
| 959735 | 12 | 67 | 90 | 95 | 0.1 |
| 959764 | 4 | 32 | 80 | 92 | 0.1 |
| 959765 | 1 | 59 | 88 | 93 | 0.1 |
| 959784 | 3 | 35 | 75 | 90 | 0.1 |
| 959785 | 27 | 72 | 92 | 96 | 0.03 |
| 959794 | 0 | 0 | 53 | 83 | 0.3 |
| 959864 | 26 | 61 | 84 | 91 | 0.05 |
| 959885 | 49 | 81 | 95 | 96 | <0.01 |
| 959914 | 7 | 43 | 76 | 89 | 0.1 |
| 959964 | 17 | 55 | 83 | 91 | 0.1 |

TABLE 58
Multi-dose assay of 3-10-3 cEt gapmers in A431 cells

| | PNPLA3 % Inhibition | | | | |
|---|---|---|---|---|---|
| Compound | 15.6 | 62.5 | 250 | 1,000 | IC₅₀ |
| Number | nM | nM | nM | nM | (μM) |
| 915609 | 0 | 73 | 95 | 97 | 0.1 |
| 959456 | 66 | 90 | 97 | 98 | <0.01 |
| 959666 | 29 | 60 | 89 | 97 | 0.04 |
| 959676 | 15 | 44 | 81 | 93 | 0.1 |
| 959686 | 71 | 92 | 97 | 97 | <0.01 |
| 959695 | 40 | 75 | 91 | 93 | 0.02 |
| 959696 | 21 | 81 | 90 | 92 | 0.03 |
| 959706 | 81 | 95 | 98 | 98 | <0.01 |
| 959725 | 8 | 55 | 76 | 84 | 0.1 |
| 959726 | 0 | 59 | 88 | 91 | 0.1 |
| 959736 | 46 | 84 | 94 | 98 | <0.01 |
| 959766 | 22 | 57 | 83 | 94 | 0.1 |
| 959776 | 1 | 53 | 87 | 93 | 0.1 |
| 959815 | 31 | 67 | 89 | 91 | 0.03 |
| 959865 | 6 | 49 | 84 | 91 | 0.1 |
| 959875 | 34 | 74 | 91 | 92 | 0.02 |
| 959935 | 22 | 55 | 84 | 94 | 0.1 |
| 959955 | 0 | 55 | 83 | 89 | 0.1 |
| 959985 | 29 | 71 | 88 | 93 | 0.03 |

TABLE 59
Multi-dose assay of 3-10-3 cEt gapmers in A431 cells

| | PNPLA3 % Inhibition | | | | |
|---|---|---|---|---|---|
| Compound | 15.6 | 62.5 | 250 | 1,000 | IC₅₀ |
| Number | nM | nM | nM | nM | (μM) |
| 915609 | 37 | 80 | 96 | 99 | 0.02 |
| 959356 | 40 | 71 | 87 | 88 | 0.02 |
| 959417 | 25 | 58 | 83 | 92 | 0.1 |
| 959437 | 65 | 88 | 94 | 95 | <0.01 |
| 959667 | 37 | 69 | 90 | 95 | 0.02 |
| 959677 | 29 | 56 | 82 | 92 | 0.05 |
| 959687 | 51 | 79 | 93 | 97 | <0.01 |
| 959697 | 51 | 75 | 93 | 95 | <0.01 |
| 959707 | 81 | 94 | 98 | 98 | <0.01 |
| 959727 | 71 | 92 | 96 | 96 | <0.01 |
| 959737 | 45 | 75 | 89 | 94 | <0.01 |
| 959767 | 47 | 76 | 93 | 96 | <0.01 |
| 959797 | 32 | 59 | 87 | 94 | 0.04 |
| 959856 | 13 | 35 | 67 | 80 | 0.1 |
| 959876 | 38 | 75 | 89 | 90 | 0.02 |
| 959877 | 40 | 81 | 89 | 94 | <0.01 |
| 959956 | 25 | 25 | 66 | 85 | 0.1 |
| 960006 | 13 | 40 | 68 | 83 | 0.1 |
| 960007 | 24 | 59 | 88 | 91 | 0.05 |

### Example 3: Tolerability of Modified Oligonucleotides Targeting Human PNPLA3 in BALB/c Mice

BALB/c mice are a multipurpose mouse model frequently utilized for safety and efficacy testing. The mice were treated with antisense oligonucleotides selected from studies described above and evaluated for changes in the levels of various plasma chemistry markers.

Ionis oligonucleotides selected from the studies above were conjugated with 3′-THA-C₆-GalNAc₃-(3R,5S)—5-(hydroxymethyl) pyrrolidin-3-ol phosphate endcap (henceforth referred to as 3′-THA).

### Treatment

Groups of 6- to 7-week-old male mice were injected subcutaneously once with 200 mg/kg of modified oligonucleotides. One group of male BALB/c mice was injected with PBS. Mice were euthanized 72-96 hours after the single dose and plasma was harvested for further analysis.

To evaluate the effect of modified oligonucleotides on liver function, plasma levels of transaminases were measured using an automated clinical chemistry analyzer (Beckman Coulter AU480, Brea, CA). Modified oligonucleotides that caused changes in the levels of transaminases outside the expected range for antisense oligonucleotides were excluded in further studies. The oligonucleotides which were considered tolerable in this study and were selected for further evaluation are presented in the Table below. ‘Parent Oligo’ indicates the Ionis oligonucleotide that has been described in the studies above and that was conjugated with 3′-THA and tested in this study.

TABLE 60
Antisense oligonucleotides in BALB/c mouse study

| Compound | Parent oligo |
|---|---|
| ID | ID |
| 975746 | 916339 |
| 975747 | 912941 |
| 975748 | 916306 |
| 975755 | 916332 |
| 975760 | 912848 |
| 975764 | 916298 |
| 975766 | 916552 |
| 975767 | 916789 |
| 975768 | 916602 |
| 975770 | 912874 |
| 975771 | 916333 |
| 975772 | 916780 |
| 975775 | 916672 |
| 975777 | 916558 |
| 975780 | 916607 |
| 975783 | 916338 |
| 975788 | 912847 |
| 975790 | 916778 |
| 975792 | 912870 |
| 975794 | 916802 |
| 975797 | 916637 |
| 975799 | 912732 |
| 975800 | 912733 |
| 975803 | 912813 |
| 975804 | 912823 |
| 975805 | 912834 |
| 975806 | 912855 |
| 975807 | 912856 |
| 975808 | 912864 |
| 975809 | 912871 |
| 975810 | 912872 |
| 975811 | 912875 |
| 975813 | 912931 |
| 975814 | 912934 |
| 975815 | 912936 |
| 975816 | 912938 |
| 975817 | 912943 |
| 975820 | 912988 |
| 975822 | 915486 |
| 975829 | 915619 |
| 975836 | 915780 |
| 975840 | 915989 |
| 975844 | 916151 |
| 975849 | 916292 |
| 975850 | 916299 |
| 975851 | 916303 |
| 975852 | 916309 |
| 975853 | 916310 |
| 975854 | 916312 |
| 975855 | 916318 |
| 975856 | 916324 |
| 975857 | 916331 |
| 975858 | 916334 |
| 975859 | 916335 |
| 975860 | 916336 |
| 975861 | 916549 |
| 975862 | 916550 |
| 975864 | 916563 |
| 975865 | 916564 |
| 975866 | 916568 |
| 975868 | 916571 |
| 975869 | 916575 |
| 975870 | 916580 |
| 975871 | 916581 |
| 975873 | 916586 |
| 975875 | 916601 |
| 975878 | 916624 |
| 975879 | 916625 |
| 975880 | 916636 |
| 975881 | 916638 |
| 975883 | 916670 |
| 975886 | 916711 |
| 975887 | 916716 |
| 975888 | 916774 |
| 975889 | 916781 |
| 975890 | 916782 |
| 975891 | 916788 |
| 975893 | 916815 |
| 975894 | 916816 |
| 975895 | 916817 |
| 975896 | 916818 |
| 975897 | 916822 |
| 975898 | 916845 |
| 994288 | 959455 |
| 994289 | 960010 |
| 994290 | 959361 |
| 994291 | 959271 |

### Example 4: Effect of Antisense Inhibition of PNPLA3 in Transgenic Mouse Model

A PNPLA3 transgenic mouse model from wild-type C57BL/6 generated by the University of California, Irvine was used. The mouse model comprises a genomic construct containing the entire PNPLA3 gene fosmid, generously provided by the University of Washington. The efficacy of Ionis oligonucleotides was evaluated in this model.

### Treatment

Transgenic mice were maintained on a 12-hour light/dark cycle and were fed ad libitum normal Purina mouse chow. Animals were acclimated for at least 7 days in the research facility before initiation of the experiment. Antisense oligonucleotides (ASOs) were prepared in buffered saline (PBS) and sterilized by filtering through a 0.2 micron filter. Oligonucleotides were dissolved in 0.9% PBS for injection.

The hPNPLA3 Tg mice were divided into groups of 2 mice each. Groups received subcutaneous injections of Ionis oligonucleotide at a dose of 2.5 mg/kg on days 1 and 8. One group of 4 mice received subcutaneous injections of PBS on days 1 and 8. The saline-injected group served as the control group to which oligonucleotide-treated groups were compared.

### RNA Analysis

On day 10, RNA was extracted from liver for real-time PCR analysis of measurement of mRNA expression of PNPLA3. Primer probe sets RTS36070 and RTS36075 were both used to measure PNPLA3 mRNA levels. Results are presented as percent change of mRNA, relative to PBS control, normalized with RIBOGREEN®. As presented in the Table below, treatment with Ionis antisense oligonucleotides resulted in significant reduction of PNPLA3 mRNA in comparison to the PBS control. ‘0’ indicates that the oligonucleotides did not inhibit mRNA expression.

TABLE 61
Percent inhibition of PNPLA3 mRNA in the transgenic
mice liver relative to the PBS control

| | Inhibition | Inhibition |
|---|---|---|
| | (%) | (%) |
| | measured | measured |
| Compound | with | with |
| ID | RTS36070 | RTS36075 |
| 975746 | 99 | 99 |
| 975747 | 99 | 99 |
| 975748 | 98 | 98 |
| 975755 | 99 | 99 |
| 975760 | 96 | 97 |
| 975764 | 75 | 83 |
| 975766 | 99 | 99 |
| 975767 | 98 | 98 |
| 975768 | 98 | 98 |
| 975770 | 97 | 97 |
| 975771 | 98 | 99 |
| 975772 | 96 | 96 |
| 975775 | 90 | 91 |
| 975777 | 85 | 89 |
| 975780 | 44 | 63 |
| 975783 | 87 | 90 |
| 975788 | 0 | 26 |
| 975790 | 0 | 0 |
| 975792 | 9 | 34 |
| 975794 | 44 | 50 |
| 975797 | 0 | 0 |
| 975799 | 0 | 0 |
| 975800 | 0 | 5 |
| 975803 | 68 | 68 |
| 975804 | 11 | 38 |
| 975805 | 0 | 0 |
| 975806 | 0 | 0 |
| 975807 | 0 | 0 |
| 975808 | 47 | 58 |
| 975809 | 0 | 19 |
| 975810 | 12 | 22 |
| 975811 | 19 | 32 |
| 975813 | 36 | 39 |
| 975814 | 48 | 54 |
| 975815 | 78 | 77 |
| 975816 | 56 | 56 |
| 975817 | 84 | 86 |
| 975820 | 35 | 45 |
| 975822 | 0 | 0 |
| 975829 | 98 | 98 |
| 975836 | 85 | 91 |
| 975840 | 19 | 44 |
| 975844 | 21 | 31 |
| 975849 | 88 | 89 |
| 975850 | 41 | 48 |
| 975851 | 5 | 18 |
| 975852 | 24 | 41 |
| 975853 | 0 | 0 |
| 975854 | 0 | 0 |
| 975855 | 0 | 0 |
| 975856 | 45 | 31 |
| 975857 | 73 | 67 |
| 975858 | 58 | 40 |
| 975860 | 92 | 92 |
| 975861 | 66 | 49 |
| 975862 | 46 | 36 |
| 975864 | 16 | 21 |
| 975865 | 0 | 0 |
| 975866 | 40 | 41 |
| 975868 | 56 | 48 |
| 975869 | 30 | 19 |
| 975870 | 0 | 14 |
| 975871 | 0 | 0 |
| 975875 | 75 | 73 |
| 975878 | 18 | 12 |
| 975879 | 7 | 0 |
| 975880 | 0 | 0 |
| 975881 | 54 | 54 |
| 975883 | 77 | 80 |
| 975886 | 18 | 28 |
| 975887 | 49 | 57 |
| 975888 | 10 | 9 |
| 975889 | 90 | 91 |
| 975890 | 96 | 98 |
| 975891 | 97 | 98 |
| 975893 | 95 | 95 |
| 975894 | 85 | 87 |
| 975895 | 89 | 89 |
| 975896 | 91 | 89 |
| 975898 | 94 | 95 |
| 975897 | 96 | 97 |
| 975873 | 99 | 99 |
| 994288 | 99 | 99 |
| 994289 | 98 | 99 |
| 994290 | 98 | 99 |
| 994291 | 95 | 95 |
| 975859 | 95 | 96 |

### Example 5: Tolerability of Modified Oligonucleotides Targeting Human PNPLA3 in CD1 Mice

CD1® mice (Charles River, MA) are a multipurpose mice model, frequently utilized for safety and efficacy testing. The mice were treated with Jonis antisense oligonucleotides selected from studies described above and evaluated for changes in the levels of various plasma chemistry markers.

Ionis oligonucleotides selected from the studies above were conjugated with 5′-Trishexylamino-(THA)-C₆GalNAC3 endcap (henceforth referred to as 5′-THA). The Jonis oligonucleotides tested are presented in the Table below. ‘Unconjugated parent ION No.’ refers to the Sonis oligonucleotide described in the in vitro studies above with the same sequence. I3′-THA counterpart ION No.' refers to the 3′-THA conjugated oligonucleotide with the same sequence and evaluated in the mice studies above.

TABLE 62
5′-THA oligonucleotides tested in CD1 mice tolerability study

| | Unconjugated | 3′-THA | | SEQ |
|---|---|---|---|---|
| Compound | parent ION | counterpart | | ID |
| ID | No. | ION No | Sequence | NO |
| 975591 | 916339 | 975746 | GGATATATTGGGCTCA | 1512 |
| 975592 | 912941 | 975747 | TTGCATTGCATAGCCT | 182 |
| 975593 | 916306 | 975748 | GTGTACTTTAGGCTCC | 598 |
| 975600 | 916332 | 975755 | CACAATGACATCATGG | 1020 |
| 975605 | 912848 | 975760 | CGTTTTTAGTAGTCAA | 141 |
| 975611 | 916552 | 975766 | CCTTTTATTTCCGTTA | 1024 |
| 975612 | 916789 | 975767 | GTAATATTCAGACCAG | 899 |
| 975613 | 916602 | 975768 | CTAGTAAATGCTTGTC | 330 |
| 975615 | 912874 | 975770 | ATACTTTTGGCAAGGC | 217 |
| 975616 | 916333 | 975771 | CTTTATTCAATGTGGC | 1089 |
| 975617 | 916780 | 975772 | AGAAATTGCAGTGCCC | 1665 |
| 975674 | 915619 | 975829 | GACTTTAGGGCAGATG | 1400 |
| 975704 | 916335 | 975859 | TAATTCTACCTGTGTC | 1227 |
| 975718 | 916586 | 975873 | AACTTTGCAGCCTATC | 605 |
| 975735 | 916782 | 975890 | CTTAGAAATTGCAGTG | 408 |
| 975736 | 916788 | 975891 | TAATATTCAGACCAGG | 830 |
| 975738 | 916815 | 975893 | CAATTCTAGACATGGC | 1313 |
| 975742 | 916822 | 975897 | TATGACATTTCAGAGT | 410 |
| 975743 | 916845 | 975898 | GTAAAGATGTGAGTGA | 618 |
| 994282 | 959455 | 994288 | TTGGATATATTGGGCT | 1982 |
| 994283 | 960010 | 994289 | AGACATATGACATTTC | 1745 |
| 994284 | 959361 | 994290 | TTTTTAGTAGTCAAGG | 1757 |
| 994285 | 959271 | 994291 | GTTGAAGGATGGATGG | 1748 |

### Treatment

Groups of four CD1 mice each were weekly injected subcutaneously with 15 mg/kg of Ionis oligonucleotides for 6 weeks, with one loading dose at day 4 (total 8 doses). One group of male CD1 mice was injected subcutaneously for 6 weeks with PBS. Mice were euthanized 48 hours after the last dose, and organs and plasma were harvested for further analysis.

### Plasma Chemistry Markers

To evaluate the effect of Ionis oligonucleotides on liver and kidney function, plasma levels of transaminases (ALT and AST), albumin, total bilirubin, and creatinine were measured at week 3 using an automated clinical chemistry analyzer (Beckman Coulter AU480, Brea, CA). The results are presented in the Table below. Ionis oligonucleotides that caused changes in the levels of any of the liver or kidney function markers outside the expected range for antisense oligonucleotides were excluded in further studies.

TABLE 63
Plasma chemistry marker levels in CD1 mice at week 3

| | | | | Total | |
|---|---|---|---|---|---|
| | Albumin | ALT | AST | bilirubin | Creatinine |
| | (g/dL) | (IU/L) | (IU/L) | (mg/dL) | (mg/dL) |
| PBS | 2.9 | 31 | 64 | 0.4 | 0.1 |
| 975611 | 2.7 | 640 | 385 | 0.3 | 0.1 |
| 994282 | 2.4 | 76 | 83 | 0.3 | 0.1 |
| 975592 | 3.0 | 786 | 942 | 0.5 | 0.1 |
| 975600 | 2.7 | 334 | 431 | 0.3 | 0.1 |
| 975591 | 2.6 | 62 | 115 | 0.4 | 0.1 |
| 975718 | 2.4 | 1717 | 2183 | 1.2 | 0.1 |
| 994284 | 2.7 | 41 | 97 | 0.3 | 0.1 |
| 994283 | 2.8 | 216 | 154 | 0.3 | 0.1 |
| 975616 | 3.0 | 69 | 137 | 0.3 | 0.1 |
| 975612 | 2.7 | 47 | 218 | 0.4 | 0.1 |
| 975674 | 2.9 | 134 | 114 | 0.4 | 0.1 |
| 975613 | 2.8 | 60 | 277 | 0.3 | 0.1 |
| 975593 | 2.7 | 429 | 405 | 0.4 | 0.1 |
| 975736 | 2.9 | 46 | 63 | 0.2 | 0.2 |
| 975735 | 2.5 | 46 | 79 | 0.2 | 0.1 |
| 975742 | 2.6 | 152 | 96 | 0.2 | 0.1 |
| 975615 | 2.9 | 207 | 189 | 0.4 | 0.1 |
| 975617 | 2.9 | 65 | 70 | 0.3 | 0.1 |
| 975605 | 2.9 | 67 | 92 | 0.3 | 0.1 |
| 975704 | 2.4 | 33 | 61 | 0.2 | 0.1 |
| 975738 | 2.6 | 43 | 67 | 0.2 | 0.1 |
| 975743 | 2.9 | 119 | 126 | 0.4 | 0.1 |
| 994285 | 2.8 | 400 | 353 | 0.2 | 0.1 |

### Hematology Assays

Blood obtained from selected mouse groups at week 6 were sent to IDEXX BioResearch for measurement of platelet count. The results are presented in the tables below. Ionis oligonucleotides that caused changes in the platelet count outside the expected range for antisense oligonucleotides were excluded in further studies.

TABLE 64
Platelet count in CD1 mice

| | Platelet |
|---|---|
| | (×10³/μL) |
| PBS | 1067 |
| 975605 | 1202 |
| 975612 | 1200 |
| 975613 | 1417 |
| 975616 | 1178 |
| 975617 | 922 |
| 975674 | 618 |
| 975591 | 941 |
| 975743 | 1127 |
| 994282 | 1384 |
| 994284 | 1255 |
| 975704 | 939 |
| 975735 | 1039 |
| 975736 | 1116 |
| 975738 | 1126 |
| 975742 | 808 |

### Example 6: Tolerability of Modified Oligonucleotides Targeting Human PNPLA3 in Sprague-Dawley Rats

Sprague-Dawley rats are a multipurpose model used for safety and efficacy evaluations. The rats were treated with Ionis antisense oligonucleotides from the studies described in the Examples above and evaluated for changes in the levels of various plasma chemistry markers.

### Treatment

Male Sprague-Dawley rats were maintained on a 12-hour light/dark cycle and fed ad libitum with Purina normal rat chow, diet 5001. Groups of 4 Sprague-Dawley rats each were weekly injected subcutaneously with 15 mg/kg of Ionis oligonucleotide for 6 weeks, with one loading dose on day 4 (total 8 doses). Forty eight hours after the last dose, rats were euthanized and organs and plasma were harvested for further analysis.

### Plasma Chemistry Markers

To evaluate the effect of Ionis oligonucleotides on hepatic function, plasma levels of transaminases were measured using an automated clinical chemistry analyzer (Beckman Coulter AU480, Brea, CA). Plasma levels of ALT (alanine transaminase) and AST (aspartate transaminase) were measured and the results are presented in the Table below expressed in IU/L. Plasma levels of bilirubin, creatinine, albumin, and BUN were also measured using the same clinical chemistry analyzer and the results are also presented in the Table below expressed in mg/dL. Ionis oligonucleotides that caused changes in the levels of any markers of liver function outside the expected range for antisense oligonucleotides were excluded in further studies.

TABLE 65
Plasma chemistry markers in Sprague-Dawley rats

| | | | | Total | | |
|---|---|---|---|---|---|---|
| | Albumin | ALT | AST | bilirubin | Creatinine | BUN |
| | (g/dL) | (IU/L) | (IU/L) | (mg/dL) | (mg/dL) | (mg/dL) |
| PBS | 3 | 35 | 81 | 0.2 | 0.2 | 12 |
| 975591 | 3 | 57 | 161 | 0.2 | 0.3 | 14 |
| 975605 | 4 | 62 | 176 | 0.3 | 0.2 | 14 |
| 975612 | 3 | 106 | 153 | 0.2 | 0.3 | 13 |
| 975613 | 3 | 32 | 94 | 0.2 | 0.2 | 12 |
| 975616 | 4 | 31 | 106 | 0.2 | 0.3 | 13 |
| 975617 | 3 | 49 | 263 | 0.2 | 0.2 | 12 |
| 975735 | 3 | 44 | 128 | 0.2 | 0.2 | 14 |
| 975736 | 3 | 73 | 293 | 0.3 | 0.3 | 14 |
| 994282 | 3 | 41 | 135 | 0.1 | 0.3 | 12 |
| 994284 | 3 | 32 | 95 | 0.1 | 0.2 | 13 |

### Kidney Function

To evaluate the effect of Ionis oligonucleotides on kidney function, urinary levels of protein and creatinine were measured using an automated clinical chemistry analyzer (Beckman Coulter AU480, Brea, CA). The ratios of total protein to creatinine are presented in the Table below. Ionis oligonucleotides that caused changes in the levels of the ratio outside the expected range for antisense oligonucleotides were excluded in further studies.

TABLE 66
Total protein to creatinine ratio in Sprague-Dawley rats

| | |
|---|---|
| PBS | 1.5 |
| 975591 | 2.0 |
| 975605 | 1.6 |
| 975612 | 1.9 |
| 975613 | 2.3 |
| 975616 | 2.0 |
| 975617 | 1.4 |
| 975735 | 2.2 |
| 975736 | 1.1 |
| 994282 | 2.1 |
| 994284 | 2.1 |

### Organ Weights

Liver, heart, spleen and kidney weights were measured at the end of the study, and are presented in the Table below. Ionis oligonucleotides that caused any changes in organ weights outside the expected range for antisense oligonucleotides were excluded from further studies.

TABLE 67
Organ weights (g)

| | Liver | Kidney | Spleen |
|---|---|---|---|
| Saline | 16 | 3 | 1 |
| 975591 | 16 | 4 | 1 |
| 975605 | 21 | 3 | 1 |
| 975612 | 12 | 3 | 1 |
| 975613 | 16 | 3 | 1 |
| 975616 | 15 | 3 | 1 |
| 975617 | 19 | 4 | 2 |
| 975735 | 14 | 4 | 1 |
| 975736 | 15 | 3 | 1 |
| 994282 | 14 | 3 | 1 |
| 994284 | 15 | 3 | 1 |

### Example 7: Effect of Antisense Inhibition of PNPLA3 in Transgenic Mouse Model

Ionis oligonucleotides were tested in a multi-dose assay in the hPNPLA3 Tg model.

### Treatment

Transgenic mice were maintained on a 12-hour light/dark cycle and were fed ad libitum normal Purina mouse chow. Animals were acclimated for at least 7 days in the research facility before initiation of the experiment. Antisense oligonucleotides (ASOs) were prepared in buffered saline (PBS) and sterilized by filtering through a 0.2 micron filter. Oligonucleotides were dissolved in 0.9% PBS for injection.

### Study 1

The hPNPLA3 Tg mice were divided into groups of 4 mice each. Groups received subcutaneous injections of Ionis oligonucleotide at a weekly dose of 5 mg/kg, 1 mg/kg, or 0.25 mg/kg administered on days 1, 5, 8, 15, and 23. One group of 4 mice received subcutaneous injections of PBS on days 1, 5, 8, 15, and 23. The saline-injected group served as the control group to which oligonucleotide-treated groups were compared.

### RNA Analysis

On day 26, RNA was extracted from liver for real-time PCR analysis of measurement of mRNA expression of PNPLA3. Primer probe sets RTS36070 and RTS36075 were both used to measure PNPLA3 mRNA levels. Results are presented as percent change of mRNA, relative to PBS control, normalized with RIBOGREEN®. As presented in the Table below, treatment with Jonis antisense oligonucleotides resulted in significant dose-dependent reduction of PNPLA3 mRNA in comparison to the PBS control.

TABLE 68
Percent inhibition of PNPLA3 mRNA in the transgenic
mice liver relative to the PBS control

| | | Inhibition | Inhibition | |
|---|---|---|---|---|
| | | measured | measured | |
| | | by | by | |
| | mg/kg/wk | RTS36070 | RTS36075 | EC₅₀ (μg/g) |
| 975605 | 5 | 93 | 90 | 2.2 |
| | 1 | 66 | 57 | |
| | 0.25 | 45 | 46 | |
| 975612 | 5 | 98 | 99 | 3.1 |
| | 1 | 89 | 88 | |
| | 0.25 | 34 | 44 | |
| 975613 | 5 | 98 | 97 | 1.0 |
| | 1 | 87 | 85 | |
| | 0.25 | 58 | 56 | |
| 975616 | 5 | 93 | 93 | 0.5 |
| | 1 | 85 | 87 | |
| | 0.25 | 60 | 63 | |
| 975617 | 5 | 97 | 97 | 0.3 |
| | 1 | 76 | 78 | |
| | 0.25 | 55 | 53 | |
| 975735 | 5 | 97 | 98 | 1.5 |
| | 1 | 74 | 75 | |
| | 0.25 | 29 | 33 | |
| 975736 | 5 | 98 | 98 | 0.9 |
| | 1 | 73 | 71 | |
| | 0.25 | 44 | 45 | |
| 994282 | 5 | 98 | 98 | 0.2 |
| | 1 | 91 | 80 | |
| | 0.25 | 62 | 58 | |
| 994284 | 5 | 99 | 100 | 0.3 |
| | 1 | 89 | 88 | |
| | 0.25 | 53 | 47 | |

### Study 2

The hPNPLA3 Tg mice were divided into groups of 4 mice each. Groups received subcutaneous injections of Ionis oligonucleotide at a weekly dose of 5 mg/kg, 2.5 mg/kg, 1 mg/kg, 0.5 mg/kg, or 0.25 mg/kg administered on days 1, 5, 8, 15, and 23. One group of 4 mice received subcutaneous injections of PBS on days 1, 5, 8, 15, and 23. The saline-injected group served as the control group to which oligonucleotide-treated groups were compared.

### RNA Analysis

On day 26, RNA was extracted from liver for real-time PCR analysis of measurement of mRNA expression of PNPLA3. Primer probe sets RTS36070 and RTS36075 were both used to measure PNPLA3 mRNA levels. Results are presented as percent change of mRNA, relative to PBS control, normalized with RIBOGREEN®. As presented in the Table below, treatment with Ionis antisense oligonucleotides resulted in significant dose-dependent reduction of PNPLA3 mRNA in comparison to the PBS control.

TABLE 69
Percent inhibition of PNPLA3 mRNA in the transgenic
mice liver relative to the PBS control

| | | Inhibition | Inhibition | | |
|---|---|---|---|---|---|
| | | measured | measured | | |
| | | by | by | | |
| | mg/kg/wk | RTS36070 | RTS36075 | EC₅₀ (μg/g) | EC₉₀ (μg/g) |
| 975612 | 5 | 96 | 97 | 1.0 | 8.6 |
| | 2.5 | 98 | 98 | | |
| | 1 | 95 | 96 | | |
| | 0.5 | 82 | 83 | | |
| | 0.25 | 43 | 44 | | |
| 975613 | 5 | 99 | 99 | 0.9 | 7.7 |
| | 2.5 | 99 | 99 | | |
| | 1 | 91 | 91 | | |
| | 0.5 | 82 | 83 | | |
| | 0.25 | 69 | 74 | | |
| | 5 | 96 | 96 | 1.0 | 9.4 |
| | 2.5 | 94 | 93 | | |
| 975616 | 1 | 89 | 89 | | |
| | 0.5 | 81 | 81 | | |
| | 0.25 | 73 | 60 | | |

### Example 8: Effect of Modified Oligonucleotides Targeting Human PNPLA3 in Cynomolgus Monkeys

Cynomolgus monkeys were treated with Ionis antisense oligonucleotides selected from studies described in the Examples above. Antisense oligonucleotide tolerability was evaluated.

### Treatment

Prior to the study, the monkeys were kept in quarantine during which the animals were observed daily for general health. The monkeys were 2-4 years old and weighed 2-4 kg. Nine groups of 5 randomly assigned male cynomolgus monkeys each were injected subcutaneously with Ionis oligonucleotide or PBS in a clock-wise rotation between four different sites on the back. The monkeys were dosed twice per week (days 1, 5, 9, and 14) for the first 2 weeks, and then subsequently once a week for 10 weeks with 10 mg/kg of Ionis oligonucleotide on days 21, 28, 35, 42, 49, 56, 63, 70, 77, and 84. A control group of 5 cynomolgus monkeys was injected with PBS in a similar manner and served as the control group.

During the study period, the monkeys were observed twice daily for signs of illness or distress. Any animal experiencing more than momentary or slight pain or distress due to the treatment, injury or illness was treated by the veterinary staff with approved analgesics or agents to relieve the pain after consultation with the Study Director. Any animal in poor health or in a possible moribund condition was identified for further monitoring and possible euthanasia. Scheduled euthanasia of the animals was conducted on day 86 approximately 48 hours after the last dose by exsanguination while under deep anesthesia. The protocols described in the Example were approved by the Institutional Animal Care and Use Committee (IACUC).

### Body and Organ Weight Measurements

To evaluate the effect of Ionis oligonucleotides on the overall health of the animals, body and organ weights were measured. Body weights and organ weights were measured on day 86 and the data is presented in the Table below. The results indicate that effect of treatment with antisense oligonucleotides on body and organ weights was within the expected range for antisense oligonucleotides. Specifically, treatment with ION 945616 was well tolerated in terms of the body and organ weights of the monkeys.

TABLE 70
Final body and organ weights in cynomolgus monkey

| | | | | Liver with |
|---|---|---|---|---|
| | Body Wt | Spleen | Kidney | gallbladder |
| | (kg) | (g) | (g) | (g) |
| PBS Control | 2797 | 2.6 | 13.1 | 53 |
| 994284 | 2789 | 3.3 | 14.7 | 69 |
| 975605 | 2685 | 4.1 | 12.2 | 58 |
| 975616 | 2868 | 3.1 | 12.9 | 63 |
| 994282 | 2782 | 4.4 | 12.1 | 62 |
| 975613 | 2704 | 3.0 | 13.5 | 60 |
| 975617 | 2761 | 3.8 | 14.1 | 61 |
| 975735 | 2765 | 4.1 | 15.5 | 67 |
| 975736 | 2844 | 3.0 | 14.1 | 66 |
| 975612 | 2711 | 2.8 | 13.2 | 60 |

### Liver Function

To evaluate the effect of Ionis oligonucleotides on hepatic function, blood samples were collected from all the study groups on day 86. The monkeys were fasted overnight prior to blood collection. Blood was collected in tubes without anticoagulant for serum separation. The tubes were kept at room temperature for a minimum of 90 minutes and then centrifuged at 3000 rpm for 10 minutes to obtain serum. Levels of various liver function markers were measured using a Toshiba 200FR NEO chemistry analyzer (Toshiba Co., Japan). Plasma levels of ALT and AST were measured and the results are presented in the Table below, expressed in IU/L. Bilirubin, a liver function marker, was similarly measured and is presented in the Table below, expressed in mg/dL. The results indicate that antisense oligonucleotides had no effect on liver function outside the expected range for antisense oligonucleotides.

TABLE 71
Liver function markers in cynomolgus monkey plasma

| | ALT | AST | Bilirubin | Albumin |
|---|---|---|---|---|
| | (IU/L) | (IU/L) | (mg/dL) | (g/dL) |
| PBS Control | 38 | 55 | 0.2 | 4.3 |
| 994284 | 64 | 48 | 0.2 | 3.7 |
| 975605 | 48 | 54 | 0.3 | 4.0 |
| 975616 | 54 | 57 | 0.3 | 3.9 |
| 994282 | 89 | 53 | 0.3 | 4.0 |
| 975613 | 60 | 71 | 0.4 | 4.0 |
| 975617 | 65 | 61 | 0.3 | 4.0 |
| 975735 | 59 | 79 | 0.3 | 4.1 |
| 975736 | 70 | 56 | 0.3 | 3.9 |
| 975612 | 61 | 66 | 0.3 | 3.9 |

### Kidney Function

To evaluate the effect of Ionis oligonucleotides on kidney function, blood samples were collected from all the study groups on day 86. The monkeys were fasted overnight prior to blood collection. Blood was collected in tubes without anticoagulant for serum separation. The tubes were kept at room temperature for a minimum of 90 minutes and then centrifuged at 3000 rpm for 10 minutes to obtain serum. Levels of BUN and creatinine were measured using a Toshiba 200FR NEO chemistry analyzer (Toshiba Co., Japan). Results are presented in the Table below, expressed in mg/dL.

The plasma chemistry data indicate that most of the Ionis oligonucleotides did not have any effect on the kidney function outside the expected range for antisense oligonucleotides.

TABLE 72
Plasma BUN and creatinine levels
(mg/dL) in cynomolgus monkeys

| | BUN | Creatinine |
|---|---|---|
| PBS Control | 23 | 0.8 |
| 994284 | 24 | 0.8 |
| 975605 | 27 | 0.7 |
| 975616 | 21 | 0.8 |
| 994282 | 24 | 0.8 |
| 975613 | 23 | 0.9 |
| 975617 | 21 | 0.7 |
| 975735 | 20 | 0.8 |
| 975736 | 23 | 0.8 |
| 975612 | 20 | 0.8 |

### Hematology

To evaluate any effect of Ionis oligonucleotides in cynomolgus monkeys on hematologic parameters, blood samples of approximately 0.5 mL of blood was collected from each of the available study animals on day 86. The samples were collected in tubes containing K₂-EDTA. Samples were analyzed for red blood cell (RBC) count, white blood cells (WBC) count, individual white blood cell counts, such as that of monocytes, neutrophils, lymphocytes, as well as for platelet count, hemoglobin content and hematocrit, using an ADVIA2120i hematology analyzer (Siemens, USA).

The data indicate the oligonucleotides did not cause any changes in hematologic parameters outside the expected range for antisense oligonucleotides at this dose.

TABLE 73
Blood cell counts in cynomolgus monkeys

| | RBC | Platelets | WBC | Neutrophils | Lymphocytes | Monocytes |
|---|---|---|---|---|---|---|
| | (×10⁶/μL) | (×10³/μL) | (×10³/μL) | (×10³/μL) | (×10³/μL) | (×10³/μL) |
| PBS | 6.0 | 342 | 12 | 3.2 | 7.8 | 0.3 |
| Control | | | | | | |
| 994284 | 6.0 | 410 | 10 | 2.7 | 6.7 | 0.3 |
| 975605 | 5.8 | 326 | 10 | 4.8 | 4.5 | 0.4 |
| 975616 | 6.0 | 362 | 10 | 3.4 | 5.8 | 0.3 |
| 994282 | 5.8 | 359 | 10 | 3.9 | 5.5 | 0.3 |
| 975613 | 5.5 | 327 | 8 | 2.6 | 5.5 | 0.2 |
| 975617 | 6.1 | 358 | 10 | 3.1 | 6.4 | 0.3 |
| 975735 | 5.9 | 241 | 13 | 5.4 | 6.6 | 0.4 |
| 975736 | 5.8 | 360 | 10 | 3.5 | 6.4 | 0.2 |
| 975612 | 6.2 | 421 | 11 | 5.1 | 5.7 | 0.2 |

TABLE 74
Hematologic parameters in cynomolgus monkeys

| | Hemoglobin | HCT |
|---|---|---|
| | (g/dL) | (%) |
| PBS Control | 14 | 49 |
| 994284 | 14 | 48 |
| 975605 | 14 | 46 |
| 975616 | 14 | 49 |
| 994282 | 14 | 47 |
| 975613 | 13 | 46 |
| 975617 | 14 | 49 |
| 975735 | 14 | 48 |
| 975736 | 14 | 48 |
| 975612 | 14 | 49 |

### Pro-Inflammatory Proteins Analysis

To evaluate any inflammatory effect of Ionis oligonucleotides in cynomolgus monkeys, blood samples were taken for analysis. The monkeys were fasted overnight prior to blood collection. Approximately 1.5 mL of blood was collected from each animal and put into tubes without anticoagulant for serum separation. The tubes were kept at room temperature for a minimum of 90 min and then centrifuged at 3,000 rpm for 10 min at room temperature to obtain serum. C-reactive protein (CRP), which is synthesized in the liver and which serves as a marker of inflammation, and complement C3 were measured using a Toshiba 200FR NEO chemistry analyzer (Toshiba Co., Japan).

### Example 9: Measurement of Viscosity of Antisense Oligonucleotides Targeting Human PNPLA3

The viscosity of select antisense oligonucleotides from the studies described above was measured with the aim of screening out antisense oligonucleotides which have a viscosity of more than 40 centipoise (cP). Oligonucleotides having a viscosity greater than 40 cP would have less than optimal viscosity.

Oligonucleotides (32-35 mg) were weighed into a glass vial, 120 μL of water was added and the antisense oligonucleotide was dissolved into solution by heating the vial at 50° C. Part (75 μL) of the pre-heated sample was pipetted to a micro-viscometer (Cambridge). The temperature of the micro-viscometer was set to 25° C. and the viscosity of the sample was measured. Another part (20 μL) of the pre-heated sample was pipetted into 10 mL of water for UV reading at 260 nM at 85° C. (Cary UV instrument). The results are presented in the Table below, where the concentration of each antisense oligonucleotide was 200 mg/ml, and indicate that most of the antisense oligonucleotides solutions are optimal in their viscosity under the criterion stated above.

TABLE 75
Viscosity of antisense oligonucleotides at 200 mg/mL

| | Viscosity (cP) |
|---|---|
| 994284 | 21 |
| 975605 | 19 |
| 975616 | 20 |
| 994282 | 30 |
| 975613 | 24 |
| 975617 | 22 |
| 975735 | 15 |
| 975736 | 49 |
| 975612 | 25 |

### Example 10: Design of Oligonucleotides at the Site of ION 975616

Additional antisense oligonucleotides were designed targeting a PNPLA3 nucleic acid that overlap the target site of ION 916333, which is the unconjugated version of ION 975616, and with different chemical modifications and motifs.

The newly designed chimeric antisense oligonucleotides in the Tables below were designed as 3-10-3 cEt gapmers or deoxy, MOE, and cEt oligonucleotides. The 3-10-3 cEt gapmers are 16 nucleosides in length, wherein the central gap segment comprises of ten 2′-deoxynucleosides and is flanked by wing segments on the 5′ direction and the 3′ direction comprising three nucleosides. Each nucleoside in the 5′ wing segment and each nucleoside in the 3′ wing segment has a cEt sugar modification. The internucleoside linkages throughout each gapmer are phosphorothioate (P═S) linkages. All cytosine residues throughout each gapmer are 5-methylcytosines. The deoxy, MOE and (S)-cEt oligonucleotides are 16 nucleosides in length wherein the nucleoside have either a MOE sugar modification, an (S)-cEt sugar modification, or a deoxy modification. The ‘Chemistry’ column describes the sugar modifications of each oligonucleotide. ‘k’ indicates an (S)-cEt sugar modification; ‘d’ indicates deoxyribose; the number after the ‘d’ indicates the number of deoxyribose; and ‘e’ indicates a MOE modification. The internucleoside linkages throughout each gapmer are phosphorothioate (P═S) linkages. All cytosine residues throughout each gapmer are 5-methylcytosines. “Start site” indicates the 5′-most nucleoside to which the gapmer is targeted in the human gene sequence (SEQ ID NO: 2).

TABLE 76
Modified oligonucleotides targeting human PNPLA3

| Start | Stop | | Compound | | SEQ |
|---|---|---|---|---|---|
| Site | Site | Sequence | Number | Chemistry | ID NO |
| 5599 | 5614 | TCAATGTGGCTTCTAG | 995553 | kkk-d10-kkk | 2170 |
| 5600 | 5615 | TTCAATGTGGCTTCTA | 959437 | kkk-d10-kkk | 2089 |
| 5601 | 5616 | ATTCAATGTGGCTTCT | 959438 | kkk-d10-kkk | 2171 |
| 5602 | 5617 | TATTCAATGTGGCTTC | 959439 | kkk-d10-kkk | 2172 |
| 5603 | 5618 | TTATTCAATGTGGCTT | 959440 | kkk-d10-kkk | 1705 |
| 5603 | 5618 | TTATTCAATGTGGCTT | 995696 | k-d10-kekek | 1705 |
| 5603 | 5618 | TTATTCAATGTGGCTT | 995906 | kk-d9-eeekk | 1705 |
| 5603 | 5618 | TTATTCAATGTGGCTT | 996116 | kk-d9-ekeke | 1705 |
| 5604 | 5619 | TTTATTCAATGTGGCT | 959441 | kkk-d10-kkk | 1765 |
| 5604 | 5619 | TTTATTCAATGTGGCT | 995697 | k-d10-kekek | 1765 |
| 5604 | 5619 | TTTATTCAATGTGGCT | 995907 | kk-d9-eeekk | 1765 |
| 5604 | 5619 | TTTATTCAATGTGGCT | 996117 | kk-d9-ekeke | 1765 |
| 5605 | 5620 | CTTTATTCAATGTGGC | 916333 | kkk-d10-kkk | 1089 |
| 5605 | 5620 | CTTTATTCAATGTGGC | 995698 | k-d10-kekek | 1089 |
| 5605 | 5620 | CTTTATTCAATGTGGC | 995908 | kk-d9-eeekk | 1089 |
| 5605 | 5620 | CTTTATTCAATGTGGC | 996118 | kk-d9-ekeke | 1089 |
| 5605 | 5620 | CTTTATTCAATGTGGC | 996277 | kek-d9-eekk | 1089 |
| 5606 | 5621 | ACTTTATTCAATGTGG | 916334 | kkk-d10-kkk | 1158 |
| 5606 | 5621 | ACTTTATTCAATGTGG | 995699 | k-d10-kekek | 1158 |
| 5606 | 5621 | ACTTTATTCAATGTGG | 995909 | kk-d9-eeekk | 1158 |
| 5606 | 5621 | ACTTTATTCAATGTGG | 996119 | kk-d9-ekeke | 1158 |
| 5607 | 5622 | TACTTTATTCAATGTG | 959442 | kkk-d10-kkk | 1825 |
| 5608 | 5623 | TTACTTTATTCAATGT | 959443 | kkk-d10-kkk | 1885 |

The oligonucleotides were tested in a series of experiments. Cultured A-431 cells at a density of 10,000 cells per well were treated using free uptake with modified oligonucleotides diluted to different concentrations. After a treatment period of approximately 48 hours, PNPLA3 mRNA levels were measured as previously described using the Human PNPLA3 primer-probe set RTS36070. PNPLA3 mRNA levels were adjusted according to total RNA content, as measured by RIBOGREEN®. The IC₅₀ ratios of the assays are presented in the tables below, which is the ratio of the IC₅₀ of a benchmark oligonucleotide to the IC₅₀ of the oligonucleotide. Hence, a bigger value of the ratio indicates that the oligonucleotide is more active than the benchmark.

TABLE 77
Efficacy of modified oligonucleotides targeting human PNPLA3

| Start | Stop | Compound | | IC₅₀ |
|---|---|---|---|---|
| Site | Site | Number | Chemistry | ratio |
| 5600 | 5615 | 959437 | kkk-d10-kkk | 1.42 |
| 5601 | 5616 | 959438 | kkk-d10-kkk | 0.49 |
| 5602 | 5617 | 959439 | kkk-d10-kkk | 0.36 |
| 5603 | 5618 | 959440 | kkk-d10-kkk | 0.55 |
| 5603 | 5618 | 995906 | kk-d9-eeekk | 1.42 |
| 5604 | 5619 | 959441 | kkk-d10-kkk | 1.66 |
| 5605 | 5620 | 916333 | kkk-d10-kkk | 1.96 |
| 5605 | 5620 | 995908 | kk-d9-eeekk | 0.70 |
| 5606 | 5621 | 916334 | kkk-d10-kkk | 0.95 |
| 5606 | 5621 | 995909 | kk-d9-eeekk | 1.47 |

### Example 11: Screen and Selection of Antisense Oligonucleotide to Human PNPLA3

S-constrained ethyl (cEt)-modified 16-mer antisense oligonucleotides (ASOs) targeting the human PNPLA3 gene were screened and tested for potency in human HepG2 cells, delivered by electroporation. A human cEt ASO (5′-GAGTTAAGTGCTGGAC-3′; SEQ ID NO: 115) was selected for all subsequent pharmacology studies. Specificity of the target knockdown was demonstrated using a chemistry-matched scrambled control ASO (5′-GGCCAATACGCCGTCA-3′; SEQ ID NO: 2173).

HepG2 cells were purchased from ATCC® (Manassas, VA). After thawing, cells were plated in T-75 flasks and grown in Minimum Essential Medium (MEM) containing 10% Fetal Bovine Serum (FBS) (HyClone Laboratories, Logan, UT).

Cells were seeded in 6-well plates or in 24-well plates with cover slips for Oil Red O (ORO) staining. Then cells were incubated with MEM (FBS 2%) and transfected with control or PNPLA3 ASOs (1 μM) or with control siRNA or PNPLA3 siRNA (10 nM) for 24 h using lipofectamine 3000 (Thermo Scientific, Waltham, MA) according to the manufacturer's instructions. Thereafter, cells were incubated with MEM (no FBS) for 24 h and a new transfection was performed. Control siRNA was a mixture of two negative control siRNA molecules (Ambion by Thermo Scientific, Waltham, MA). PNPLA3 siRNA was a mixture of three siRNA molecules (5′-GGUCCUCUCAGAUCUUGUGtt-3′ (SEQ ID NO: 2170); 5′-GGAGUGAGUGACAACGUACtt-3′ (SEQ ID NO: 2171); 5′-GGUUCUUGGAAGAGAAGGGtt-3′ (SEQ ID NO: 2172) (Ambion by Thermo Scientific, Waltham, MA).

For Oil Red O (ORO) staining, pictures were obtained using Axio KS 400 Imaging System and Axio Vision 4.8 Software (Zeiss, Oberkochen, Germany) at 100× magnification. ORO-stained area was quantified by BioPix iQ 2.1.4 software (BioPix AB, Gothenburg, Sweden).

Total RNA was isolated from the cells using RNeasy Mini Kit (Qiagen, Valencia, CA). First-strand complementary DNAs were synthesized from RNA using a reverse-transcription kit (Applied Biosystems, Foster City, CA). PNPLA3 and D-ACTIN mRNA expression were assessed in HepG2 cells transfected with control ASO. PNPLA3 ASO. control siRNA or PNPLA3 siRNA by real-time quantitative polymerase chain reaction (PCR). TaqMan probes (PNPLA3 probe: Hs00228747 ml; β-ACTIN probe: Hs01060665_g1) and master mix (Life Technologies, Carlsbad, CA) were used according to the manufacturer's protocol. Real-time quantitative PCR assay was performed on a CFX Real-Time PCR Detection System (Bio-Rad, Hercules, CA).

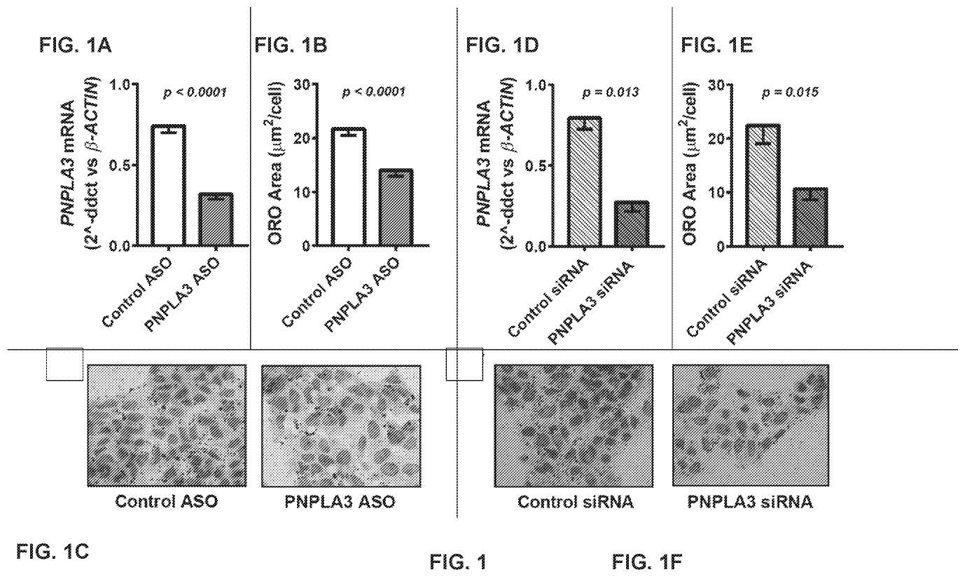

To test whether knock-down of PNPLA3 148M mutant protein affects the intracellular neutral fat content, PNPLA3 was suppressed in HepG2 cells (homozygous for the PNPLA3 148M/M mutation) by using ASO and siRNA as described herein. An approximately 70% reduction of the endogenous PNPLA3 mRNA expression was obtained using a PNPLA3 ASO (FIG. 1A). When intracellular neutral fat was examined by ORO staining, a 40% reduction of the intracellular lipid content with the PNPLA3 ASO was observed (FIG. 1B,C). To confirm these data by an independent approach, PNPLA3 was suppressed in these cells by using siRNA and observed consistent results (FIG. 1D-F).

### Example 12: Antisense Oligonucleotide Treatment of Wild Type and PNPLA3 I148M Knock-In Mice

The materials and methods used in the Examples herein are further described in Lind6n et al., Molecular Metabolism 22:49-61, 2019, incorporated by reference herein in its entirety.

S-constrained ethyl (cEt)-modified 16-mer ASOs targeting the mouse Pnpla3 gene were screened and tested for potency in primary mouse embryonic cortical neurons via free uptake. A potent mouse (5′-TATTTTTGGTGTATCC-3′; SEQ ID NO: 2174) cEt ASO lead was selected for all subsequent pharmacological studies. This mouse Pnpla3 ASO was modified by 5′-conjugation with triantennary N-acetylgalactosamine (GalNAc₃) to further enhance the liver cell targeting in vivo following subcutaneous administration. The specificity of target knockdown was demonstrated using a chemistry-matched scrambled control GalNAc3-conjugated ASO (5′-GGCCAATACGCCGTCA-3′; SEQ ID NO: 2175). The control GalNAc₃-conjugated ASO did not affect body weight-gain, liver weight, plasma alanine aminotransferase (ALT) or liver triglyceride content when dosed at 10 mg/kg/week for six weeks in mice fed a NASH-inducing diet (D09100301, Research Diets, New Brunswick, NJ) as compared to saline vehicle controls.

All animal experiments were performed with humane care and were approved by the Gothenburg Ethics Committee for Experimental Animals in Sweden. The holding facility has received full accreditation from the Association for Assessment and Accreditation of Laboratory Animal Care (AAALAC).

The human PNPLA3 I148M mutation was introduced into the mouse Pnpla3 gene by replacing the isoleucine codon with a methionine codon in amino acid position 148 of the mouse Pnpla3 gene using homologous recombination. Founders were backcrossed with C57BL/6N females to generate heterozygous Pnpla3 1481/M mice. Sequence-verified heterozygous Pnpla3 1481/M mice were intercrossed to generate experimental homozygous Pnpla3 148M/M and wild-type littermates (Pnpla3 148I/I) as control mice for the dietary challenge and ASO pharmacology studies. All experimental animals were verified to have the correct genotype using PCR before the study began and verified again using PCR after termination. A few experimental animals were also verified by cDNA sequencing. All animals were housed in transparent Makrolon cages with aspen wood chip bedding and nesting material, and the temperature-(21±1° C.) and humidity (50±10%) of the holding facility were controlled. The mice had free access to tap water and food and were on a 12-h day/night cycle.

Female Pnpla3 148M/M (n=21) and wild-type littermates (n=19) (6-8 weeks of age) were fed a high-sucrose diet (70% sucrose diet; TD98090, Envigo, Huntingdon, UK) for 15 weeks. Female mice were used in this experiment to replicate the established model by Smagris et al. Hepatology 61(1):108-118, 2015, who used female animals in their high-sucrose diet experiments. In addition, in a pilot experiment, female mice accumulated more triglycerides in the liver compared to male mice when fed this diet. In addition, female mice fed the high-sucrose diet accumulated more liver triglycerides compared to mice fed a regular chow diet containing (energy percentage) 12% fat, 62% carbohydrates, and 26% protein, with a total energy content of 3 kcal/g (R3; Lactamin, Kimstad, Sweden). After 5 weeks of feeding on the high-sucrose diet, liver lipid levels were assessed using a magnetic resonance imaging (MRI)-derived marker, proton-density fat fraction (PDFF). The mice were then assigned to GalNAc₃-conjugated ASO study groups (n=9-12 animals/group) based on randomized stratification for body weight and liver lipid contents before the treatment was initiated. During the last 8 weeks of the study, groups of mice were dosed with either the control ASO or Pnpla3 ASO (5 mg/kg/week administered by two subcutaneous injections per week with saline as vehicle). After 6 weeks of ASO dosing, liver lipid levels were again assessed using MRI. Before unfasted mice were euthanized at 8:00-10:00 AM, the mice were metabolically synchronized for 24 h by withdrawing the food from 8:00 AM to 8:00 PM and then allowed free access to the food again from 8:00 PM to 8:00 AM. Mice were euthanized with isoflurane (Forene, Abbot Scandinavia AB, Sweden), blood was collected and plasma isolated, livers were collected, and pieces (same position in the left lateral lobe for all mice) were fixed with 4% formaldehyde in PBS for histology or snap-frozen in liquid N₂ and stored at −80° C.

Male Pnpla3148 M/M mice (n=17) and wild-type littermates (n=17) (6-8 weeks of age) were fed a diet high in fat (40%, containing 18% trans-fat), carbohydrates (40%, containing 20% fructose) and cholesterol (2%) (NASH diet; D09100301, Research Diets, New Brunswick, NJ) for 26 weeks. In a separate experiment, liver Pnpla3 mRNA, triglyceride content as well as plasma ALT levels were found to be elevated in wild-type male mice fed the NASH-inducing diet as compared to mice fed a regular chow diet. The mice were assigned to GalNAc₃-conjugated ASO study groups (n=8-9 mice/group) based on body weight, and during the last 14 weeks, the mice were dosed with either the control ASO or Pnpla3 ASO (5 mg/kg/week administered by two subcutaneous injections per week with saline as vehicle). Before unfasted mice were euthanized at 8:00-10:00 AM, the mice were metabolically synchronized for 24 h as described above. Mice were euthanized with isoflurane (Forene, Abbot Scandinavia AB, Sweden), blood was collected and plasma isolated, livers were collected, and pieces (same position in the left lateral lobe for all mice) were fixed with 4% formaldehyde in PBS for histology or snap-frozen in liquid N2 and stored at −80° C.

### Example 13: Effect of Pnpla3 ASO on Liver Steatosis in Wild-Type and I148M Mice Fed a High-Sucrose Diet

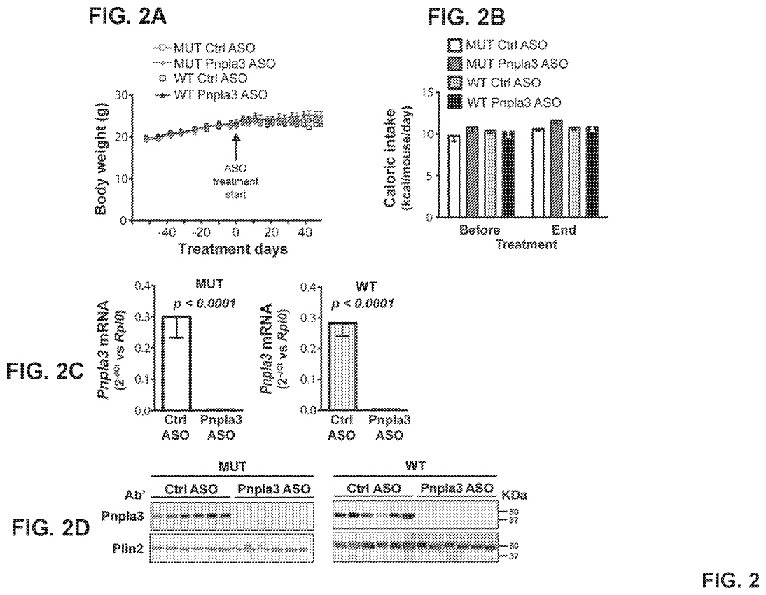

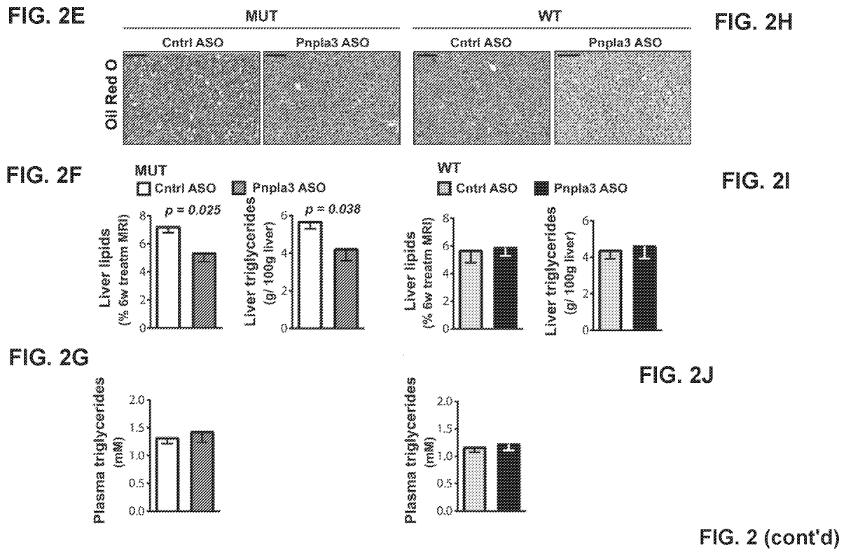

To drive hepatic lipogenesis and to evaluate the impact of Pnpla3 silencing on hepatic fat accumulation, homozygous Pnpla3 148M/M (mutant) knock-in female mice and wild-type littermates were fed a high-sucrose diet (70%) for 15 weeks as described in Example 11. During the last 8 weeks of the experiment, mice of the two genotypes were treated with GalNAc₃-conjugated Pnpla3 or GalNAc₃-conjugated control ASOs. No differences in body weight gain, food intake (FIGS. 2A and B) or in ovarian white adipose tissue weight were observed between the groups. In addition, the Pnpla3 ASO treatment did not affect plasma glucose or insulin levels. Compared to the control ASO, the Pnpla3 ASO treatment markedly reduced the hepatic expression of Pnpla3 mRNA (98% reduction, p<0.0001) and levels of PNPLA3 protein on lipid droplets in both Pnpla3 mutant knock-in and wild-type mice (FIGS. 2C and D). The Pnpla3 ASO treatment did not affect the white adipose tissue expression of Pnpla3 mRNA levels.

Six weeks of the Pnpla3 ASO treatment reduced liver lipid levels by 20% in Pnpla3 mutant knock-in mice, as measured by MRI (FIG. 2F, p=0.025). After 8 weeks of treatment, the Pnpla3 mutant knock-in mice treated with the Pnpla3 ASO showed reduced liver weights, lower liver Oil Red O staining of neutral lipids (FIG. 2E), a 20% reduction (p=0.038) in liver triglyceride content, as measured by biochemical analyses (FIG. 2F), while no changes in circulating plasma triglyceride levels were observed (FIG. 2G). Interestingly, the Pnpla3 ASO treatment did not affect the liver weights, lipid level or liver triglyceride content in the wild-type mice (FIG. 2H-J). Pnpla3 mutant knock-in mice treated with the control ASO had 30% higher liver triglyceride content than the wild-type mice treated with the control ASO (Pnpla3 mutant knock-in mice=5.7±0.4 g/100 g liver, wild-type mice=4.4±0.5 g/100 g liver, p=0.046).

### Example 14: Effect of Pnpla3 ASO on Liver Inflammation and Fibrosis in Wild-Type and I148M Mice Fed a NASH-Inducing Diet

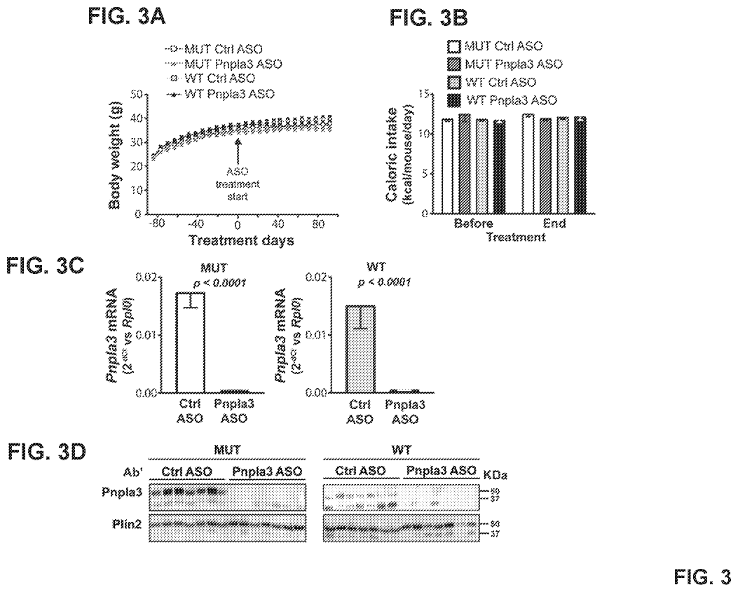

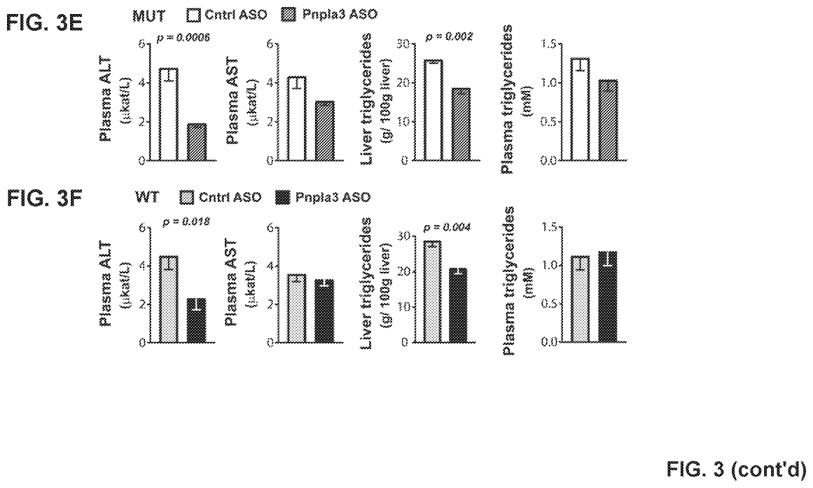

Male Pnpla3 mutant knock-in (n=17) and wild-type littermate (n=17) mice were fed a NASH-inducing diet for 26 weeks as described in Example 11. During the last 14 weeks of the experiment, mice of both genotypes were treated with a GalNAc₃-conjugated Pnpla3 or GalNAc₃-conjugated control ASO. No differences in body weight gain, food intake (FIGS. 3A and B), or in epididymal white adipose tissue weight were observed between the groups. In addition, the Pnpla3 ASO treatment did not affect plasma glucose or insulin levels. Compared to the control ASO, the Pnpla3 ASO treatment markedly reduced (97%, p<0.0001) hepatic expression of the Pnpla3 mRNA and consistently reduced levels of the Pnpla3 protein on lipid droplets in both Pnpla3 mutant knock-in and wild-type mice (FIGS. 3C and D). Using the NASH diet with a longer treatment period than in the sucrose diet study (14 weeks vs 8 weeks, respectively), the Pnpla3 ASO treatment also reduced the white adipose tissue expression of Pnpla3 mRNA levels.

The Pnpla3 ASO treatment reduced plasma ALT levels in mice of both genotypes (Pnpla3 mutant knock-in p=0.0006, wild-type p=0.018), while plasma AST was unchanged (FIGS. 3E and F). The Pnpla3 ASO treatment reduced liver weights only in the Pnpla3 mutant knock-in mice, triglyceride contents in both Pnpla3 mutant knock-in (p=0.002) and wild-type mice (p=0.004), while no changes in circulating plasma triglyceride levels were observed (FIGS. 3E and F).

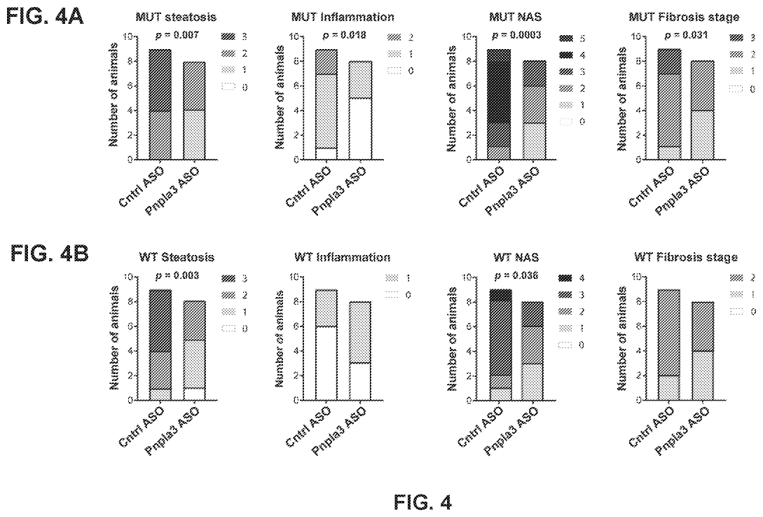

The Pnpla3 ASO treatment improved the liver steatosis score (p=0.007), lobular inflammation score (p=0.018), NAFLD activity score (NAS) (p=0.0003), and fibrosis stage (p=0.031) in Pnpla3 mutant knock-in mice (FIG. 4A) while only liver steatosis score (p=0.003) and NAS (p=0.036) were improved in the wild-type mice (FIG. 4B). Hepatocellular ballooning degeneration was not found in any of the livers.

### Example 15: Effect of Pnpla3 ASO on De Novo Lipogenesis and Palmitoleic Acid in Wild-Type and I148M Mice Fed a NASH-Inducing Diet

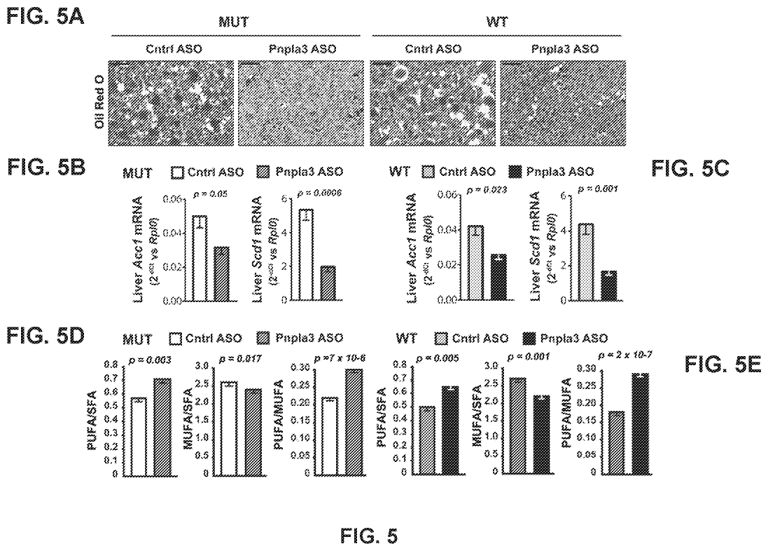

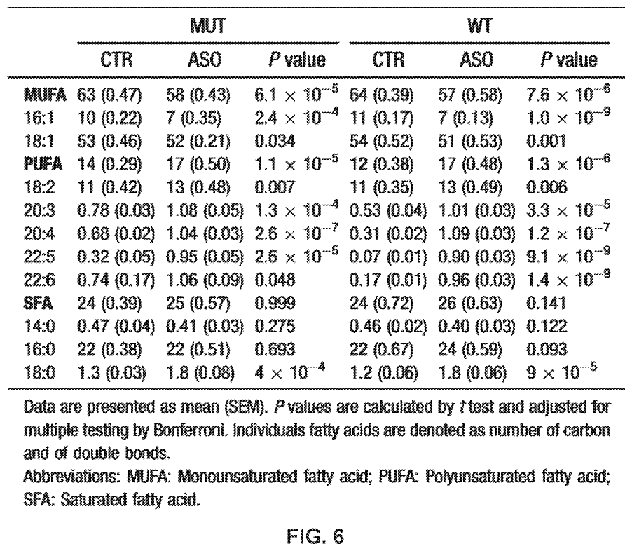

The Pnpla3 ASO treatment reduced the liver Oil Red O staining of neutral lipids in both Pnpla3 mutant knock-in and wild-type mice (FIG. 5A). The Pnpla3 ASO treatment reduced the mRNA expression of lipogenic genes, such as acetyl-CoA carboxylase 1 (Acc1) and stearoyl-CoA desaturase 1 (Scd1) in both genotypes (FIGS. 5B and C), suggesting that hepatic lipogenesis was reduced. Pnpla3 ASO treatment decreased the relative amount of monounsaturated fatty acids (MUFAs, p=6.1×10⁻⁵ and 7.6×10⁻⁶ in the mutant and wild-type, respectively) and increased polyunsaturated fatty acids (PUFAs, p=1.2×10⁻⁴ and 1.3×10⁻⁵ in the mutant and wild-type, respectively) irrespective of the genotype (FIGS. 5D-E and 6).

Specifically, the reduction in MUFAs was stronger for the palmitoleic acid (16:1), 36% (p=2.4×10⁻⁴) and 30% (1.0×10⁻⁹) reduction for the mutant and wild-type, respectively as compared with the oleic acid where the reduction was only 2% (p=0.034) and 5% (p=0.001) for the mutant and wild-type, respectively.

### Example 16: Effect of Pnpla3 ASO on Protein Levels of Haptoglobin, Mcp1 and Timp2 in Wild-Type and I148M Mice Fed a NASH-Inducing Diet

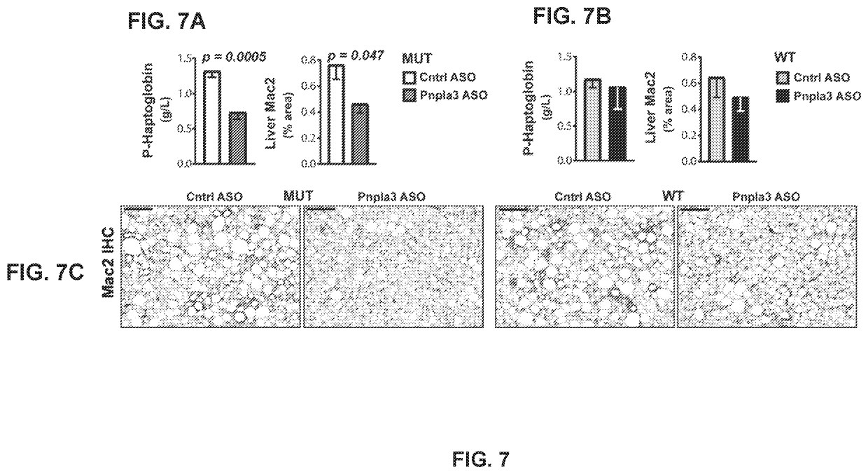

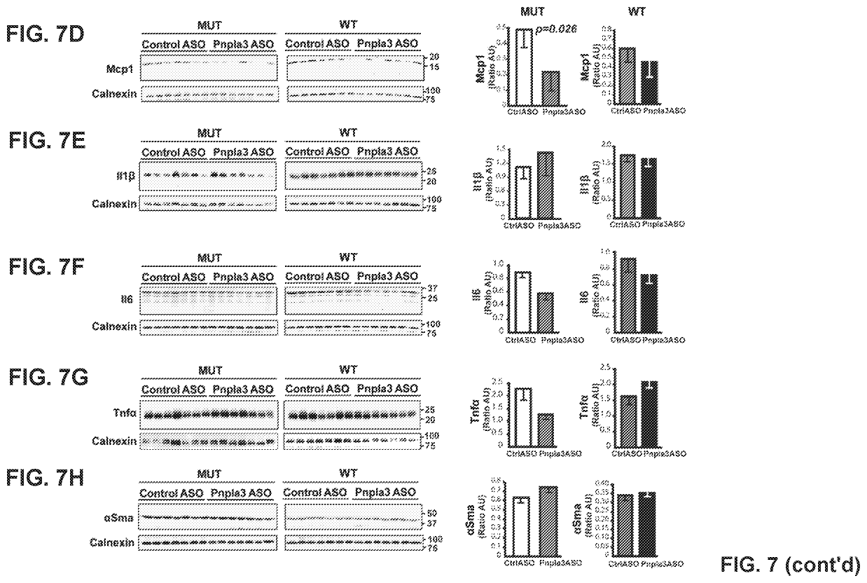

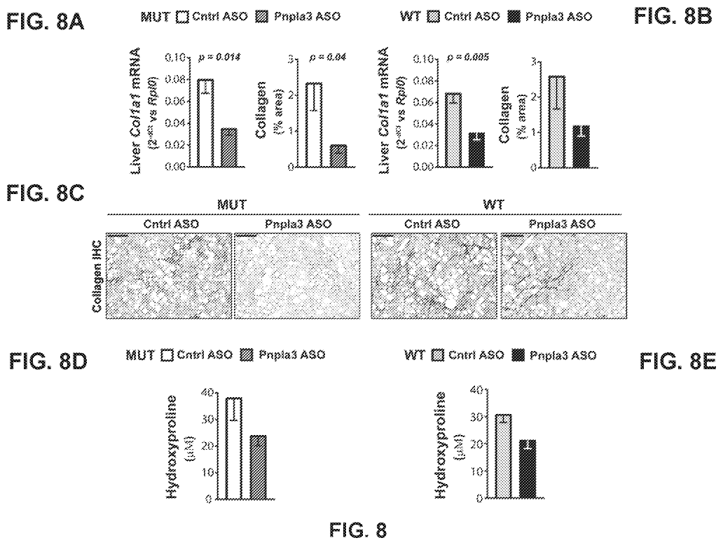

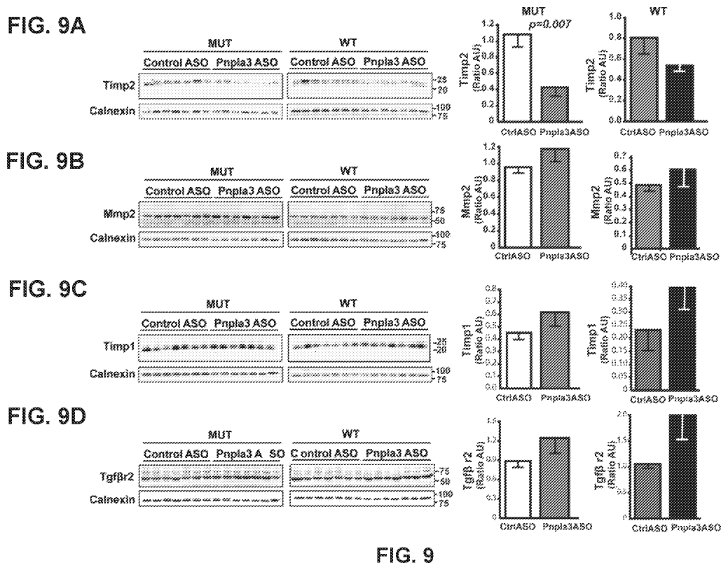

The Pnpla3 ASO treatment reduced plasma haptoglobin levels (p=0.0005) and the liver macrophage content (p=0.047) in Pnpla3 mutant knock-in mice but not in the wild-type littermates (FIG. 7A-C), suggesting that Pnpla3 suppression specifically reduced liver inflammation in the mutant mice. Pnpla3 ASO treatment reduced the liver Mcp1 (FIG. 7D) protein levels in Pnpla3 mutant knock-in mice. Pnpla3 ASO treatment did not change the liver protein expression levels of IIIβ (FIG. 7E), 116 (FIG. 7F), Tnfα (FIG. 7G), or αSma (FIG. 7H) in either genotype. The Pnpla3 ASO treatment reduced the hepatic expression of the collagen type I alpha 1 (Collal) mRNA in both Pnpla3 mutant knock-in and wild-type mice (FIGS. 8A and B). The Pnpla3 ASO treatment reduced the liver collagen, as measured by immunohistochemistry (p=0.04), in the Pnpla3 mutant knock-in mice (FIG. 8A-C). Although ASO treatment tended to decrease liver hydroxyproline levels, no significant differences were observed (FIGS. 8D and E). Pnpla3 ASO treatment reduced the liver Timp2 protein levels in Pnpla3 mutant knock-in mice (p=0.007) (FIG. 9A). Pnpla3 ASO treatment did not change the liver protein expression levels of Mmp2 (FIG. 9B), Timp1 (FIG. 9C), or Tgfβr2 (FIG. 9D) in either genotype.

1. A method of treating an individual having or at risk of having liver disease, comprising administering a compound targeted to PNPLA3 to the individual,
wherein the individual has an I148M mutation in patatin-like phospholipase domain-containing protein 3 (PNPLA3).

2. A method of reducing one or more of liver damage, hepatic steatosis, liver inflammation, liver fibrosis, and hepatic lipogenesis in an individual, comprising administering a compound targeted to PNPLA3 to the individual,
wherein the individual has an I148M mutation in patatin-like phospholipase domain-containing protein 3 (PNPLA3).

3. A method of reducing protein levels of one or more of haptoglobin, MCP1, and TIMP2 in an individual, comprising administering a compound targeted to PNPLA3 to the individual,
wherein the individual has an I148M mutation in patatin-like phospholipase domain-containing protein 3 (PNPLA3).

4. The method of claim 1, wherein the liver disease is selected from non-alcoholic fatty liver disease (NAFLD), hepatic steatosis, non-alcoholic steatohepatitis (NASH), liver cirrhosis, hepatocellular carcinoma, alcoholic liver disease, alcoholic steatohepatitis (ASH), HCV hepatitis, chronic hepatitis, hereditary hemochromatosis, or primary sclerosing cholangitis.

5. The method of claim 4, wherein the liver disease is hepatic steatosis.

6. The method of claim 2, wherein the method reduces or inhibits liver inflammation or liver fibrosis.

7. The method of claim 2, wherein reducing or inhibiting liver inflammation comprises reducing liver macrophage levels.

8. The method of claim 7, wherein the liver macrophage levels are reduced by at least 20% relative to an individual not administered the compound targeted to PNPLA3, as measured by immunohistochemical staining of a liver section of the individual.

9. The method of claim 3, wherein the protein levels of haptoglobin are reduced by at least 20% relative to an individual not administered the compound targeted to PNPLA3, as measured by a colorimetric assay of serum or plasma of the individual.

10. The method of claim 3, wherein the protein levels of MCP1 are reduced by at least 20% relative to an individual not administered the compound targeted to PNPLA3, as measured by immunoblotting of a liver sample of the individual.

11. The method of claim 3, wherein the protein levels of TIMP2 are reduced by at least 20% relative to an individual not administered the compound targeted to PNPLA3, as measured by immunoblotting of a liver sample of the individual.

12. The method of claim 1, wherein the individual has a homozygous I148M mutation in PNPLA3.

13. The method of claim 1, wherein the individual is a human individual.

14. The method of claim 1, wherein the compound targeted to PNPLA3 is an antisense compound targeted to PNPLA3.

15. The method of claim 14, wherein the antisense compound targeted to PNPLA3 is a short interfering RNA (siRNA).

16. The method of claim 14, wherein the antisense compound targeted to PNPLA3 is an antisense oligonucleotide (ASO).

17. The method of claim 14, wherein the compound targeted to PNPLA3 comprises a modified oligonucleotide 8 to 80 linked nucleosides in length having a having a nucleobase sequence comprising at least 8, at least 9, at least 10, at least 11, or at least 12 contiguous nucleobases of any of the nucleobase sequences of SEQ ID NOs: 17-2169.

18. The method of claim 14, wherein the compound targeted to PNPLA3 comprises a modified oligonucleotide 8 to 80 nucleosides in length and having a nucleobase sequence comprising the nucleobase sequence of any one of SEQ ID NOs: 17-2169.

19. The method of claim 14, wherein the compound targeted to PNPLA3 comprises a modified oligonucleotide having a nucleobase sequence consisting of any one of SEQ ID NOs: 17-2169.

20. The method of claim 14, wherein the compound targeted to PNPLA3 comprises a modified oligonucleotide 8 to 80 linked nucleosides in length, wherein the modified oligonucleotide has a nucleobase sequence comprising a portion of at least 8 contiguous nucleobases 100% complementary to an equal length portion of nucleobases 5567-5642, 5644-5731, 5567-5731, 5567-5620, 13697-13733, 20553-20676, 20664-20824, 20553-20824, or 25844-25912 of SEQ ID NO: 2, and wherein the nucleobase sequence of the modified oligonucleotide is at least 85%, at least 90%, at least 95%, or 100% complementary to SEQ ID NO: 2.

21-61. (canceled)
